SEQUENCE LISTING (111 sequences)

SEQ ID NO: 1 - RNA, 567 nt, Homo sapiens
  source 1..567: mol_type = mRNA
aaccctcctgcctgtctgctcagttcatccctagaggcagctgctccaggaacagaggtgccatgcagccccgggtactccttgttgttgccctcctggcgctcctggcctctgcccgagcttcagaggccgaggatgcctcccttctcagcttcatgcagggttacatgaagcacgccaccaagaccgccaaggatgcactgagcagcgtgcaggagtcccaggtggcccagcaggccaggggctgggtgaccgatggcttcagttccctgaaagactactggagcaccgttaaggacaagttctctgagttctgggatttggaccctgaggtcagaccaacttcagccgtggctgcctgagacctcaataccccaagtccacctgcctatccatcctgcgagctccttgggtcctgcaatctccagggctgcccctgtaggttgcttaaaagggacagtattctcagtgctctcctaccccacctcatgcctggcccccctccaggcatgctggcctcccaataaagctggacaagaagctgctatgagtaaaaaaaaaaaaaaa

SEQ ID NO: 2 - RNA, 23 nt, synthetic construct
  source 1..23: mol_type = other RNA
  modified_base 1: mod_base = OTHER; note = thymine
tcactgagaatactgtccctttt

SEQ ID NO: 3 - RNA, 23 nt, synthetic construct
  source 1..23: mol_type = other RNA
  modified_base 1: mod_base = OTHER; note = thymine
tcactgagaatactgtcccttaa

SEQ ID NO: 4 - RNA, 23 nt, synthetic construct
  source 1..23: mol_type = other RNA
  modified_base 1: mod_base = OTHER; note = thymine
tcactgagaatactgtcccttaa

SEQ ID NO: 5 - RNA, 21 nt, synthetic construct
  source 1..21: mol_type = other RNA
  modified_base 1: mod_base = OTHER; note = thymine
tcactgagaatactgtcccaa

SEQ ID NO: 6 - RNA, 21 nt, synthetic construct
  source 1..21: mol_type = other RNA
aagggacagtattctcagtga

SEQ ID NO: 7 - RNA, 19 nt, synthetic construct
  source 1..19: mol_type = other RNA
gggacagtattctcagtga

SEQ ID NO: 8 - RNA, 22 nt, synthetic construct
  source 1..22: mol_type = other RNA
  modified_base 1: mod_base = OTHER; note = thymine
taagggacagtattctcagtga

SEQ ID NO: 9 - RNA, 21 nt, synthetic construct
  source 1..21: mol_type = other RNA
  modified_base 1: mod_base = OTHER; note = 2'-O-methyl uridine 3'-phosphorothioate
  modified_base 2: mod_base = OTHER; note = 2'-fluoro cytidine 3'-phosphorothioate
  modified_base 3: mod_base = OTHER; note = 2'-O-methyl adenosine 3'-phosphorothioate
  modified_base 4: mod_base = OTHER; note = 2'-fluoro cytidine 3'-phosphodiester
  modified_base 5: mod_base = OTHER; note = 2'-O-methyl uridine 3'-phosphodiester
  modified_base 6: mod_base = OTHER; note = 2'-fluoro guanosine 3'-phosphodiester
  modified_base 7: mod_base = OTHER; note = 2'-O-methyl adenosine 3'-phosphodiester
  modified_base 8: mod_base = OTHER; note = 2'-O-methyl guanosine 3'-phosphodiester
  modified_base 9..10: mod_base = OTHER; note = 2'-O-methyl adenosine 3'-phosphodiester
  modified_base 11: mod_base = OTHER; note = 2'-O-methyl uridine 3'-phosphodiester
  modified_base 12: mod_base = OTHER; note = 2'-fluoro adenosine 3'-phosphodiester
  modified_base 13: mod_base = OTHER; note = 2'-O-methyl cytidine 3'-phosphodiester
  modified_base 14: mod_base = OTHER; note = 2'-fluoro uridine 3'-phosphodiester
  modified_base 15: mod_base = OTHER; note = 2'-O-methyl guanosine 3'-phosphodiester
  modified_base 16: mod_base = OTHER; note = 2'-fluoro uridine 3'-phosphodiester
  modified_base 17: mod_base = OTHER; note = 2'-O-methyl cytidine 3'-phosphodiester
  modified_base 18: mod_base = OTHER; note = 2'-fluoro cytidine 3'-phosphodiester
  modified_base 19: mod_base = OTHER; note = 2'-O-methyl cytidine 3'-phosphodiester
  modified_base 20: mod_base = OTHER; note = 2'-fluoro guanosine 3'-phosphorothioate
  modified_base 21: mod_base = OTHER; note = 2'-O-methyl uridine
tcactgagaatactgtcccgt

SEQ ID NO: 10 - RNA, 23 nt, synthetic construct
  source 1..23: mol_type = other RNA
  modified_base 20: mod_base = i
  modified_base 1: mod_base = OTHER; note = inverted abasic 2'-beta-D-deoxyribose 3'-phosphorothioate, where the 3'-position of the sugar is conjugated to Diglutamine diethylglycol N-Acetyl galactosamine-4-carbamoylcyclohexyl via a phosphorothioate bond
  modified_base 2: mod_base = OTHER; note = 2'-O-methyl adenosine 3'-phosphodiester
  modified_base 3: mod_base = OTHER; note = 2'-O-methyl cytidine 3'-phosphodiester
  modified_base 4..6: mod_base = OTHER; note = 2'-O-methyl guanosine 3'-phosphodiester
  modified_base 7: mod_base = OTHER; note = 2'-O-methyl adenosine 3'-phosphodiester
  modified_base 8: mod_base = OTHER; note = 2'-O-methyl cytidine 3'-phosphodiester
  modified_base 9: mod_base = OTHER; note = 2'-O-methyl adenosine 3'-phosphodiester
  modified_base 10: mod_base = OTHER; note = 2'-fluoro guanosine 3'-phosphodiester
  modified_base 11: mod_base = OTHER; note = 2'-fluoro uridine 3'-phosphodiester
  modified_base 12: mod_base = OTHER; note = 2'-fluoro adenosine 3'-phosphodiester
  modified_base 13..14: mod_base = OTHER; note = 2'-O-methyl uridine 3'-phosphodiester
  modified_base 16: mod_base = OTHER; note = 2'-O-methyl uridine 3'-phosphodiester
  modified_base 15: mod_base = OTHER; note = 2'-O-methyl cytidine 3'-phosphodiester
  modified_base 17: mod_base = OTHER; note = 2'-O-methyl cytidine 3'-phosphodiester
  modified_base 18: mod_base = OTHER; note = 2'-O-methyl adenosine 3'-phosphodiester
  modified_base 19: mod_base = OTHER; note = 2'-O-methyl guanosine 3'-phosphodiester
  modified_base 20: mod_base = OTHER; note = 2'-O-methyl uridine 3'-phosphodiester
  modified_base 21: mod_base = OTHER; note = 2'-O-methyl inosine 3'-phosphodiester
  modified_base 22: mod_base = OTHER; note = 2'-O-methyl adenosine phosphorothioate
  modified_base 23: mod_base = OTHER; note = inverted abasic 2'-beta-D-deoxyribose
nacgggacagtattctcagtnan

SEQ ID NO: 11 - RNA, 23 nt, synthetic construct
  source 1..23: mol_type = other RNA
  modified_base 1: mod_base = OTHER; note = 2'-O-methoxyethyl thymidine 3'-phosphorothioate, conjugated at the 5' end to vinyl phosphonate
  modified_base 2: mod_base = OTHER; note = 2'-fluoro cytidine 3'-phosphorothioate
  modified_base 3: mod_base = OTHER; note = 2'-O-methyl adenosine 3'-phosphodiester
  modified_base 4: mod_base = OTHER; note = 2'-O-methyl cytidine 3'-phosphodiester
  modified_base 5: mod_base = OTHER; note = 2'-O-methyl uridine 3'-phosphodiester
  modified_base 6: mod_base = OTHER; note = 2'-fluoro guanosine 3'-phosphodiester
  modified_base 7: mod_base = OTHER; note = 2'-O-methyl adenosine 3'-phosphodiester
  modified_base 8: mod_base = OTHER; note = 2'-O-methyl guanosine 3'-phosphodiester
  modified_base 9..10: mod_base = OTHER; note = 2'-O-methyl adenosine 3'-phosphodiester
  modified_base 11: mod_base = OTHER; note = 2'-O-methyl uridine 3'-phosphodiester
  modified_base 12: mod_base = OTHER; note = 2'-O-methyl adenosine 3'-phosphodiester
  modified_base 13: mod_base = OTHER; note = 2'-O-methyl cytidine 3'-phosphodiester
  modified_base 14: mod_base = OTHER; note = 2'-fluoro uridine 3'-phosphodiester
  modified_base 15: mod_base = OTHER; note = 2'-O-methyl guanosine 3'-phosphodiester
  modified_base 16: mod_base = OTHER; note = 2'-fluoro uridine 3'-phosphodiester
  modified_base 17..19: mod_base = OTHER; note = 2'-O-methyl cytidine 3'-phosphodiester
  modified_base 20: mod_base = OTHER; note = 2'-O-methyl uridine 3'-phosphodiester
  modified_base 21..22: mod_base = OTHER; note = 2'-O-methyl uridine 3'-phosphorothioate
  modified_base 23: mod_base = OTHER; note = 2'-O-methyl uridine
tcactgagaatactgtccctttt

SEQ ID NO: 12 - RNA, 23 nt, synthetic construct
  source 1..23: mol_type = other RNA
  modified_base 1: mod_base = OTHER; note = 2'-O-methoxyethyl thymidine 3'-phosphorothioate, conjugated at the 5' end to vinyl phosphonate
  modified_base 2: mod_base = OTHER; note = 2'-fluoro cytidine 3'-phosphorothioate
  modified_base 3: mod_base = OTHER; note = 2'-O-methyl adenosine 3'-phosphodiester
  modified_base 4: mod_base = OTHER; note = 2'-O-methyl cytidine 3'-phosphodiester
  modified_base 5: mod_base = OTHER; note = 2'-O-methyl uridine 3'-phosphodiester
  modified_base 6: mod_base = OTHER; note = 2'-deoxyguanosine 3'-phosphodiester
  modified_base 7: mod_base = OTHER; note = 2'-O-methyl adenosine 3'-phosphodiester
  modified_base 8: mod_base = OTHER; note = 2'-O-methyl guanosine 3'-phosphodiester
  modified_base 9..10: mod_base = OTHER; note = 2'-O-methyl adenosine 3'-phosphodiester
  modified_base 11: mod_base = OTHER; note = 2'-O-methyl uridine 3'-phosphodiester
  modified_base 12: mod_base = OTHER; note = 2'-O-methyl adenosine 3'-phosphodiester
  modified_base 13: mod_base = OTHER; note = 2'-O-methyl cytidine 3'-phosphodiester
  modified_base 14: mod_base = OTHER; note = 2'-fluoro uridine 3'-phosphodiester
  modified_base 15: mod_base = OTHER; note = 2'-O-methyl guanosine 3'-phosphodiester
  modified_base 16: mod_base = OTHER; note = 2'-fluoro uridine 3'-phosphodiester
  modified_base 17..19: mod_base = OTHER; note = 2'-O-methyl cytidine 3'-phosphodiester
  modified_base 20: mod_base = OTHER; note = 2'-O-methyl uridine 3'-phosphodiester
  modified_base 21..22: mod_base = OTHER; note = 2'-O-methyl uridine 3'-phosphorothioate
  modified_base 23: mod_base = OTHER; note = 2'-O-methyl uridine
tcactgagaatactgtccctttt

SEQ ID NO: 13 - RNA, 23 nt, synthetic construct
  source 1..23: mol_type = other RNA
  modified_base 1: mod_base = OTHER; note = 2'-O-methoxyethyl thymidine 3'-phosphorothioate, conjugated at the 5' end to vinyl phosphonate
  modified_base 2: mod_base = OTHER; note = 2'-fluoro cytidine 3'-phosphorothioate
  modified_base 3: mod_base = OTHER; note = 2'-O-methyl adenosine 3'-phosphodiester
  modified_base 4: mod_base = OTHER; note = 2'-O-methyl cytidine 3'-phosphodiester
  modified_base 5: mod_base = OTHER; note = 2'-O-methyl uridine 3'-phosphodiester
  modified_base 6: mod_base = OTHER; note = 2'-deoxyguanosine 3'-phosphorothioate
  modified_base 7: mod_base = OTHER; note = 2'-O-methyl adenosine 3'-phosphodiester
  modified_base 8: mod_base = OTHER; note = 2'-O-methyl guanosine 3'-phosphodiester
  modified_base 9..10: mod_base = OTHER; note = 2'-O-methyl adenosine 3'-phosphodiester
  modified_base 11: mod_base = OTHER; note = 2'-O-methyl uridine 3'-phosphodiester
  modified_base 12: mod_base = OTHER; note = 2'-O-methyl adenosine 3'-phosphodiester
  modified_base 13: mod_base = OTHER; note = 2'-O-methyl cytidine 3'-phosphodiester
  modified_base 14: mod_base = OTHER; note = 2'-fluoro uridine 3'-phosphodiester
  modified_base 15: mod_base = OTHER; note = 2'-O-methyl guanosine 3'-phosphodiester
  modified_base 16: mod_base = OTHER; note = 2'-fluoro uridine 3'-phosphodiester
  modified_base 17..19: mod_base = OTHER; note = 2'-O-methyl cytidine 3'-phosphodiester
  modified_base 20: mod_base = OTHER; note = 2'-O-methyl uridine 3'-phosphodiester
  modified_base 21..22: mod_base = OTHER; note = 2'-O-methyl uridine 3'-phosphorothioate
  modified_base 23: mod_base = OTHER; note = 2'-O-methyl uridine
tcactgagaatactgtccctttt

SEQ ID NO: 14 - RNA, 23 nt, synthetic construct
  source 1..23: mol_type = other RNA
  modified_base 1: mod_base = OTHER; note = 2'-O-methoxyethyl thymidine 3'-phosphorothioate, conjugated at the 5' end to vinyl phosphonate
  modified_base 2: mod_base = OTHER; note = 2'-fluoro cytidine 3'-phosphorothioate
  modified_base 3: mod_base = OTHER; note = 2'-O-methyl adenosine 3'-phosphodiester
  modified_base 4: mod_base = OTHER; note = 2'-O-methyl cytidine 3'-phosphodiester
  modified_base 5: mod_base = OTHER; note = 2'-O-methyl uridine 3'-phosphodiester
  modified_base 6: mod_base = OTHER; note = 2'-deoxyguanosine 3'-phosphodiester
  modified_base 7: mod_base = OTHER; note = 2'-O-methyl adenosine 3'-phosphodiester
  modified_base 8: mod_base = OTHER; note = 2'-O-methyl guanosine 3'-phosphodiester
  modified_base 9..10: mod_base = OTHER; note = 2'-O-methyl adenosine 3'-phosphodiester
  modified_base 11: mod_base = OTHER; note = 2'-O-methyl uridine 3'-phosphodiester
  modified_base 12: mod_base = OTHER; note = 2'-O-methyl uridine 3'-phosphodiester
  modified_base 13: mod_base = OTHER; note = 2'-O-methyl cytidine 3'-phosphodiester
  modified_base 14: mod_base = OTHER; note = 2'-deoxyuridine 3'-phosphodiester
  modified_base 15: mod_base = OTHER; note = 2'-O-methyl guanosine 3'-phosphodiester
  modified_base 16: mod_base = OTHER; note = 2'-deoxyuridine 3'-phosphodiester
  modified_base 17..19: mod_base = OTHER; note = 2'-O-methyl cytidine 3'-phosphodiester
  modified_base 20: mod_base = OTHER; note = 2'-O-methyl uridine 3'-phosphodiester
  modified_base 21..22: mod_base = OTHER; note = 2'-O-methyl uridine 3'-phosphorothioate
  modified_base 23: mod_base = OTHER; note = 2'-O-methyl uridine
tcactgagaatactgtccctttt

SEQ ID NO: 15 - RNA, 23 nt, synthetic construct
  source 1..23: mol_type = other RNA
  modified_base 1: mod_base = OTHER; note = 2'-O-methoxyethyl thymidine 3'-phosphorothioate, conjugated at the 5' end to vinyl phosphonate
  modified_base 2: mod_base = OTHER; note = 2'-fluoro cytidine 3'-phosphorothioate
  modified_base 3: mod_base = OTHER; note = 2'-O-methyl adenosine 3'-phosphodiester
  modified_base 4: mod_base = OTHER; note = 2'-O-methyl cytidine 3'-phosphodiester
  modified_base 5: mod_base = OTHER; note = 2'-O-methyl uridine 3'-phosphodiester
  modified_base 6: mod_base = OTHER; note = 2'-deoxyguanosine 3'-phosphorothioate
  modified_base 7: mod_base = OTHER; note = 2'-O-methyl adenosine 3'-phosphodiester
  modified_base 8: mod_base = OTHER; note = 2'-O-methyl guanosine 3'-phosphodiester
  modified_base 9..10: mod_base = OTHER; note = 2'-O-methyl adenosine 3'-phosphodiester
  modified_base 11: mod_base = OTHER; note = 2'-O-methyl uridine 3'-phosphodiester
  modified_base 12: mod_base = OTHER; note = 2'-O-methyl adenosine 3'-phosphodiester
  modified_base 13: mod_base = OTHER; note = 2'-O-methyl cytidine 3'-phosphodiester
  modified_base 14: mod_base = OTHER; note = 2'-deoxyuridine 3'-phosphorothioate
  modified_base 15: mod_base = OTHER; note = 2'-O-methyl guanosine 3'-phosphodiester
  modified_base 16: mod_base = OTHER; note = 2'-deoxyuridine 3'-phosphorothioate
  modified_base 17..19: mod_base = OTHER; note = 2'-O-methyl cytidine 3'-phosphodiester
  modified_base 20: mod_base = OTHER; note = 2'-O-methyl uridine 3'-phosphodiester
  modified_base 21..22: mod_base = OTHER; note = 2'-O-methyl uridine 3'-phosphorothioate
  modified_base 23: mod_base = OTHER; note = 2'-O-methyl uridine
tcactgagaatactgtccctttt

SEQ ID NO: 16 - RNA, 23 nt, synthetic construct
  source 1..23: mol_type = other RNA
  modified_base 1: mod_base = OTHER; note = 2'-O-methoxyethyl thymidine 3'-phosphorothioate, conjugated at the 5' end to vinyl phosphonate
  modified_base 2: mod_base = OTHER; note = 2'-fluoro cytidine 3'-phosphorothioate
  modified_base 3: mod_base = OTHER; note = 2'-O-methyl adenosine 3'-phosphodiester
  modified_base 4: mod_base = OTHER; note = 2'-O-methyl cytidine 3'-phosphodiester
  modified_base 5: mod_base = OTHER; note = 2'-O-methyl uridine 3'-phosphodiester
  modified_base 6: mod_base = OTHER; note = 2'-O-methyl guanosine 3'-phosphodiester
  modified_base 7: mod_base = OTHER; note = 2'-O-methyl adenosine 3'-phosphodiester
  modified_base 8: mod_base = OTHER; note = 2'-O-methyl guanosine 3'-phosphodiester
  modified_base 9..10: mod_base = OTHER; note = 2'-O-methyl adenosine 3'-phosphodiester
  modified_base 11: mod_base = OTHER; note = 2'-O-methyl uridine 3'-phosphodiester
  modified_base 12: mod_base = OTHER; note = 2'-O-methyl adenosine 3'-phosphodiester
  modified_base 13: mod_base = OTHER; note = 2'-O-methyl cytidine 3'-phosphodiester
  modified_base 14: mod_base = OTHER; note = 2'-fluoro uridine 3'-phosphodiester
  modified_base 15: mod_base = OTHER; note = 2'-O-methyl guanosine 3'-phosphodiester
  modified_base 16: mod_base = OTHER; note = 2'-O-methyl uridine 3'-phosphodiester
  modified_base 17..19: mod_base = OTHER; note = 2'-O-methyl cytidine 3'-phosphodiester
  modified_base 20: mod_base = OTHER; note = 2'-O-methyl uridine 3'-phosphodiester
  modified_base 21..22: mod_base = OTHER; note = 2'-O-methyl uridine 3'-phosphorothioate
  modified_base 23: mod_base = OTHER; note = 2'-O-methyl uridine
tcactgagaatactgtccctttt

SEQ ID NO: 17 - RNA, 23 nt, synthetic construct
  source 1..23: mol_type = other RNA
  modified_base 1: mod_base = OTHER; note = 2'-O-methoxyethyl thymidine 3'-phosphorothioate, conjugated at the 5' end to vinyl phosphonate
  modified_base 2: mod_base = OTHER; note = 2'-fluoro cytidine 3'-phosphorothioate
  modified_base 3: mod_base = OTHER; note = 2'-O-methyl adenosine 3'-phosphodiester
  modified_base 4: mod_base = OTHER; note = 2'-O-methyl cytidine 3'-phosphodiester
  modified_base 5: mod_base = OTHER; note = 2'-O-methyl uridine 3'-phosphodiester
  modified_base 6: mod_base = OTHER; note = 2'-fluoro guanosine 3'-phosphodiester
  modified_base 7: mod_base = OTHER; note = 2'-O-methyl adenosine 3'-phosphodiester
  modified_base 8: mod_base = OTHER; note = 2'-O-methyl guanosine 3'-phosphodiester
  modified_base 9: mod_base = OTHER; note = 2'-O-methoxyethyl adenosine 3'-phosphodiester
  modified_base 10: mod_base = OTHER; note = 2'-O-methyl adenosine 3'-phosphodiester
  modified_base 11: mod_base = OTHER; note = 2'-O-methyl uridine 3'-phosphodiester
  modified_base 12: mod_base = OTHER; note = 2'-O-methyl adenosine 3'-phosphodiester
  modified_base 13: mod_base = OTHER; note = 2'-O-methyl cytidine 3'-phosphodiester
  modified_base 14: mod_base = OTHER; note = 2'-fluoro uridine 3'-phosphodiester
  modified_base 15: mod_base = OTHER; note = 2'-O-methyl guanosine 3'-phosphodiester
  modified_base 16: mod_base = OTHER; note = 2'-fluoro uridine 3'-phosphodiester
  modified_base 17..19: mod_base = OTHER; note = 2'-O-methyl cytidine 3'-phosphodiester
  modified_base 20: mod_base = OTHER; note = 2'-O-methyl uridine 3'-phosphodiester
  modified_base 21..22: mod_base = OTHER; note = 2'-O-methyl uridine 3'-phosphorothioate
  modified_base 23: mod_base = OTHER; note = 2'-O-methyl uridine
tcactgagaatactgtccctttt

SEQ ID NO: 18 - RNA, 23 nt, synthetic construct
  source 1..23: mol_type = other RNA
  modified_base 1: mod_base = OTHER; note = 2'-O-methoxyethyl thymidine 3'-phosphorothioate, conjugated at the 5' end to vinyl phosphonate
  modified_base 2: mod_base = OTHER; note = 2'-fluoro cytidine 3'-phosphorothioate
  modified_base 3: mod_base = OTHER; note = 2'-O-methyl adenosine 3'-phosphodiester
  modified_base 4: mod_base = OTHER; note = 2'-O-methyl cytidine 3'-phosphodiester
  modified_base 5: mod_base = OTHER; note = 2'-O-methyl uridine 3'-phosphodiester
  modified_base 6: mod_base = OTHER; note = 2'-fluoro guanosine 3'-phosphodiester
  modified_base 7: mod_base = OTHER; note = 2'-O-methyl adenosine 3'-phosphodiester
  modified_base 8: mod_base = OTHER; note = 2'-O-methyl guanosine 3'-phosphodiester
  modified_base 9..10: mod_base = OTHER; note = 2'-O-methoxyethyl adenosine 3'-phosphodiester
  modified_base 11: mod_base = OTHER; note = 2'-O-methyl uridine 3'-phosphodiester
  modified_base 12: mod_base = OTHER; note = 2'-O-methyl adenosine 3'-phosphodiester
  modified_base 13: mod_base = OTHER; note = 2'-O-methyl cytidine 3'-phosphodiester
  modified_base 14: mod_base = OTHER; note = 2'-fluoro uridine 3'-phosphodiester
  modified_base 15: mod_base = OTHER; note = 2'-O-methyl guanosine 3'-phosphodiester
  modified_base 16: mod_base = OTHER; note = 2'-fluoro uridine 3'-phosphodiester
  modified_base 17..19: mod_base = OTHER; note = 2'-O-methyl cytidine 3'-phosphodiester
  modified_base 20: mod_base = OTHER; note = 2'-O-methyl uridine 3'-phosphodiester
  modified_base 21..22: mod_base = OTHER; note = 2'-O-methyl uridine 3'-phosphorothioate
  modified_base 23: mod_base = OTHER; note = 2'-O-methyl uridine
tcactgagaatactgtccctttt

SEQ ID NO: 19 - RNA, 23 nt, synthetic construct
  source 1..23: mol_type = other RNA
  modified_base 1: mod_base = OTHER; note = 2'-O-methoxyethyl thymidine 3'-phosphorothioate, conjugated at the 5' end to methylene phosphonate
  modified_base 2: mod_base = OTHER; note = 2'-fluoro cytidine 3'-phosphorothioate
  modified_base 3: mod_base = OTHER; note = 2'-O-methyl adenosine 3'-phosphodiester
  modified_base 4: mod_base = OTHER; note = 2'-O-methyl cytidine 3'-phosphodiester
  modified_base 5: mod_base = OTHER; note = 2'-O-methyl uridine 3'-phosphodiester
  modified_base 6: mod_base = OTHER; note = 2'-fluoro guanosine 3'-phosphodiester
  modified_base 7: mod_base = OTHER; note = 2'-O-methyl adenosine 3'-phosphodiester
  modified_base 8: mod_base = OTHER; note = 2'-O-methyl guanosine 3'-phosphodiester
  modified_base 9..10: mod_base = OTHER; note = 2'-O-methyl adenosine 3'-phosphodiester
  modified_base 11: mod_base = OTHER; note = 2'-O-methyl uridine 3'-phosphodiester
  modified_base 12: mod_base = OTHER; note = 2'-O-methyl adenosine 3'-phosphodiester
  modified_base 13: mod_base = OTHER; note = 2'-O-methyl cytidine 3'-phosphodiester
  modified_base 14: mod_base = OTHER; note = 2'-fluoro uridine 3'-phosphodiester
  modified_base 16: mod_base = OTHER; note = 2'-fluoro uridine 3'-phosphodiester
  modified_base 15: mod_base = OTHER; note = 2'-O-methyl guanosine 3'-phosphodiester
  modified_base 17..19: mod_base = OTHER; note = 2'-O-methyl cytidine 3'-phosphodiester
  modified_base 20: mod_base = OTHER; note = 2'-O-methyl uridine 3'-phosphodiester
  modified_base 21..22: mod_base = OTHER; note = 2'-O-methyl uridine 3'-phosphorothioate
  modified_base 23: mod_base = OTHER; note = 2'-O-methyl uridine
tcactgagaatactgtccctttt

SEQ ID NO: 20 - RNA, 23 nt, synthetic construct
  source 1..23: mol_type = other RNA
  modified_base 1: mod_base = OTHER; note = 2'-O-methoxyethyl thymidine 3'-phosphorothioate, conjugated at the 5' end to vinyl phosphonate
  modified_base 2: mod_base = OTHER; note = 2'-fluoro cytidine 3'-phosphorothioate
  modified_base 3: mod_base = OTHER; note = 2'-O-methyl adenosine 3'-phosphodiester
  modified_base 4: mod_base = OTHER; note = 2'-O-methyl cytidine 3'-phosphodiester
  modified_base 5: mod_base = OTHER; note = 2'-O-methyl uridine 3'-phosphodiester
  modified_base 6: mod_base = OTHER; note = 2'-deoxyguanosine 3'-phosphorothioate
  modified_base 7: mod_base = OTHER; note = 2'-O-methyl adenosine 3'-phosphodiester
  modified_base 8: mod_base = OTHER; note = 2'-O-methyl guanosine 3'-phosphodiester
  modified_base 9..10: mod_base = OTHER; note = 2'-O-methyl adenosine 3'-phosphodiester
  modified_base 11: mod_base = OTHER; note = 2'-O-methyl uridine 3'-phosphodiester
  modified_base 12: mod_base = OTHER; note = 2'-O-methyl adenosine 3'-phosphodiester
  modified_base 13: mod_base = OTHER; note = 2'-O-methyl cytidine 3'-phosphodiester
  modified_base 14: mod_base = OTHER; note = 2'-fluoro uridine 3'-phosphodiester
  modified_base 15: mod_base = OTHER; note = 2'-O-methyl guanosine 3'-phosphodiester
  modified_base 16: mod_base = OTHER; note = 2'-fluoro uridine 3'-phosphodiester
  modified_base 17..19: mod_base = OTHER; note = 2'-O-methyl cytidine 3'-phosphodiester
  modified_base 20: mod_base = OTHER; note = 2'-O-methyl uridine 3'-phosphodiester
  modified_base 21: mod_base = OTHER; note = 2'-O-methyl uridine 3'-phosphorothioate
  modified_base 22: mod_base = OTHER; note = 2'-O-methoxyethyl adenosine 3'-phosphorothioate
  modified_base 23: mod_base = OTHER; note = 2'-O-methoxyethyl adenosine
tcactgagaatactgtcccttaa

SEQ ID NO: 21 - RNA, 23 nt, synthetic construct
  source 1..23: mol_type = other RNA
  modified_base 1: mod_base = OTHER; note = 2'-O-methoxyethyl thymidine 3'-phosphorothioate, conjugated at the 5' end to vinyl phosphonate
  modified_base 2: mod_base = OTHER; note = 2'-fluoro cytidine 3'-phosphorothioate
  modified_base 3: mod_base = OTHER; note = 2'-O-methyl adenosine 3'-phosphodiester
  modified_base 4: mod_base = OTHER; note = 2'-O-methyl cytidine 3'-phosphodiester
  modified_base 5: mod_base = OTHER; note = 2'-O-methyl uridine 3'-phosphodiester
  modified_base 6: mod_base = OTHER; note = 2'-deoxyguanosine 3'-phosphorothioate
  modified_base 7: mod_base = OTHER; note = 2'-O-methyl adenosine 3'-phosphodiester
  modified_base 8: mod_base = OTHER; note = 2'-O-methyl guanosine 3'-phosphodiester
  modified_base 9..10: mod_base = OTHER; note = 2'-O-methyl adenosine 3'-phosphodiester
  modified_base 11: mod_base = OTHER; note = 2'-O-methyl uridine 3'-phosphodiester
  modified_base 12: mod_base = OTHER; note = 2'-O-methyl adenosine 3'-phosphodiester
  modified_base 13: mod_base = OTHER; note = 2'-O-methyl cytidine 3'-phosphodiester
  modified_base 14: mod_base = OTHER; note = 2'-deoxyuridine 3'-phosphorothioate
  modified_base 15: mod_base = OTHER; note = 2'-O-methyl guanosine 3'-phosphodiester
  modified_base 16: mod_base = OTHER; note = 2'-deoxyuridine 3'-phosphorothioate
  modified_base 17..19: mod_base = OTHER; note = 2'-O-methyl cytidine 3'-phosphodiester
  modified_base 20: mod_base = OTHER; note = 2'-O-methyl uridine 3'-phosphodiester
  modified_base 21: mod_base = OTHER; note = 2'-O-methyl uridine 3'-phosphorothioate
  modified_base 22: mod_base = OTHER; note = 2'-O-methoxyethyl adenosine 3'-phosphorothioate
  modified_base 23: mod_base = OTHER; note = 2'-O-methoxyethyl adenosine
tcactgagaatactgtcccttaa

SEQ ID NO: 22 - RNA, 23 nt, synthetic construct
  source 1..23: mol_type = other RNA
  modified_base 1: mod_base = OTHER; note = 2'-O-methoxyethyl thymidine 3'-phosphorothioate, conjugated at the 5' end to vinyl phosphonate
  modified_base 2: mod_base = OTHER; note = 2'-fluoro cytidine 3'-phosphorothioate
  modified_base 3: mod_base = OTHER; note = 2'-O-methyl adenosine 3'-phosphodiester
  modified_base 4: mod_base = OTHER; note = 2'-O-methyl cytidine 3'-phosphodiester
  modified_base 5: mod_base = OTHER; note = 2'-O-methyl uridine 3'-phosphodiester
  modified_base 6: mod_base = OTHER; note = 2'-deoxyguanosine 3'-phosphodiester
  modified_base 7: mod_base = OTHER; note = 2'-O-methyl adenosine 3'-phosphodiester
  modified_base 8: mod_base = OTHER; note = 2'-O-methyl guanosine 3'-phosphodiester
  modified_base 9..10: mod_base = OTHER; note = 2'-O-methyl adenosine 3'-phosphodiester
  modified_base 11: mod_base = OTHER; note = 2'-O-methyl uridine 3'-phosphodiester
  modified_base 12: mod_base = OTHER; note = 2'-O-methyl adenosine 3'-phosphodiester
  modified_base 13: mod_base = OTHER; note = 2'-O-methyl cytidine 3'-phosphodiester
  modified_base 14: mod_base = OTHER; note = 2'-fluoro uridine 3'-phosphodiester
  modified_base 15: mod_base = OTHER; note = 2'-O-methyl guanosine 3'-phosphodiester
  modified_base 16: mod_base = OTHER; note = 2'-fluoro uridine 3'-phosphodiester
  modified_base 17..19: mod_base = OTHER; note = 2'-O-methyl cytidine 3'-phosphodiester
  modified_base 20: mod_base = OTHER; note = 2'-O-methyl uridine 3'-phosphodiester
  modified_base 21: mod_base = OTHER; note = 2'-O-methyl uridine 3'-phosphorothioate
  modified_base 22: mod_base = OTHER; note = 2'-O-methoxyethyl adenosine 3'-phosphorothioate
  modified_base 23: mod_base = OTHER; note = 2'-O-methoxyethyl adenosine
tcactgagaatactgtcccttaa

SEQ ID NO: 23 - RNA, 23 nt, synthetic construct
  source 1..23: mol_type = other RNA
  modified_base 1: mod_base = OTHER; note = 2'-O-methoxyethyl thymidine 3'-phosphorothioate, conjugated at the 5' end to vinyl phosphonate
  modified_base 2: mod_base = OTHER; note = 2'-fluoro cytidine 3'-phosphorothioate
  modified_base 3: mod_base = OTHER; note = 2'-O-methyl adenosine 3'-phosphodiester
  modified_base 4: mod_base = OTHER; note = 2'-O-methyl cytidine 3'-phosphodiester
  modified_base 5: mod_base = OTHER; note = 2'-O-methyl uridine 3'-phosphodiester
  modified_base 6: mod_base = OTHER; note = 2'-deoxyguanosine 3'-phosphodiester
  modified_base 7: mod_base = OTHER; note = 2'-O-methyl adenosine 3'-phosphodiester
  modified_base 8: mod_base = OTHER; note = 2'-O-methyl guanosine 3'-phosphodiester
  modified_base 9..10: mod_base = OTHER; note = 2'-O-methyl adenosine 3'-phosphodiester
  modified_base 11: mod_base = OTHER; note = 2'-O-methyl uridine 3'-phosphodiester
  modified_base 12: mod_base = OTHER; note = 2'-O-methyl adenosine 3'-phosphodiester
  modified_base 13: mod_base = OTHER; note = 2'-O-methyl cytidine 3'-phosphodiester
  modified_base 14: mod_base = OTHER; note = 2'-deoxyuridine 3'-phosphodiester
  modified_base 15: mod_base = OTHER; note = 2'-O-methyl guanosine 3'-phosphodiester
  modified_base 16: mod_base = OTHER; note = 2'-deoxyuridine 3'-phosphodiester
  modified_base 17..19: mod_base = OTHER; note = 2'-O-methyl cytidine 3'-phosphodiester
  modified_base 20: mod_base = OTHER; note = 2'-O-methyl uridine 3'-phosphodiester
  modified_base 21: mod_base = OTHER; note = 2'-O-methyl uridine 3'-phosphorothioate
  modified_base 22: mod_base = OTHER; note = 2'-O-methoxyethyl adenosine 3'-phosphorothioate
  modified_base 23: mod_base = OTHER; note = 2'-O-methoxyethyl adenosine
tcactgagaatactgtcccttaa

SEQ ID NO: 24 - RNA, 23 nt, synthetic construct
  source 1..23: mol_type = other RNA
  modified_base 1: mod_base = OTHER; note = 2'-O-methoxyethyl thymidine 3'-phosphorothioate, conjugated at the 5' end to vinyl phosphonate
  modified_base 2: mod_base = OTHER; note = 2'-fluoro cytidine 3'-phosphorothioate
  modified_base 3: mod_base = OTHER; note = 2'-O-methyl adenosine 3'-phosphodiester
  modified_base 4: mod_base = OTHER; note = 2'-O-methyl cytidine 3'-phosphodiester
  modified_base 5: mod_base = OTHER; note = 2'-O-methyl uridine 3'-phosphodiester
  modified_base 6: mod_base = OTHER; note = 2'-deoxyguanosine 3'-phosphorothioate
  modified_base 7: mod_base = OTHER; note = 2'-O-methyl adenosine 3'-phosphodiester
  modified_base 8: mod_base = OTHER; note = 2'-O-methyl guanosine 3'-phosphodiester
  modified_base 9: mod_base = OTHER; note = 2'-O-methoxyethyl adenosine 3'-phosphodiester
  modified_base 10: mod_base = OTHER; note = 2'-O-methyl adenosine 3'-phosphodiester
  modified_base 11: mod_base = OTHER; note = 2'-O-methyl uridine 3'-phosphodiester
  modified_base 12: mod_base = OTHER; note = 2'-O-methyl adenosine 3'-phosphodiester
  modified_base 13: mod_base = OTHER; note = 2'-O-methyl cytidine 3'-phosphodiester
  modified_base 14: mod_base = OTHER; note = 2'-fluoro uridine 3'-phosphodiester
  modified_base 15: mod_base = OTHER; note = 2'-O-methyl guanosine 3'-phosphodiester
  modified_base 16: mod_base = OTHER; note = 2'-fluoro uridine 3'-phosphodiester
  modified_base 17..19: mod_base = OTHER; note = 2'-O-methyl cytidine 3'-phosphodiester
  modified_base 20: mod_base = OTHER; note = 2'-O-methyl uridine 3'-phosphodiester
  modified_base 21: mod_base = OTHER; note = 2'-O-methyl uridine 3'-phosphorothioate
  modified_base 22: mod_base = OTHER; note = 2'-O-methoxyethyl adenosine 3'-phosphorothioate
  modified_base 23: mod_base = OTHER; note = 2'-O-methoxyethyl adenosine
tcactgagaatactgtcccttaa

SEQ ID NO: 25 - RNA, 23 nt, synthetic construct
  source 1..23: mol_type = other RNA
  modified_base 1: mod_base = OTHER; note = 2'-O-methoxyethyl thymidine 3'-phosphorothioate, conjugated at the 5' end to vinyl phosphonate
  modified_base 2: mod_base = OTHER; note = 2'-fluoro cytidine 3'-phosphorothioate
  modified_base 3: mod_base = OTHER; note = 2'-O-methyl adenosine 3'-phosphodiester
  modified_base 4: mod_base = OTHER; note = 2'-O-methyl cytidine 3'-phosphodiester
  modified_base 5: mod_base = OTHER; note = 2'-O-methyl uridine 3'-phosphodiester
  modified_base 6: mod_base = OTHER; note = 2'-deoxyguanosine 3'-phosphodiester
  modified_base 7: mod_base = OTHER; note = 2'-O-methyl adenosine 3'-phosphodiester
  modified_base 8: mod_base = OTHER; note = 2'-O-methyl guanosine 3'-phosphodiester
  modified_base 9: mod_base = OTHER; note = 2'-O-methoxyethyl adenosine 3'-phosphodiester
  modified_base 10: mod_base = OTHER; note = 2'-O-methyl adenosine 3'-phosphodiester
  modified_base 11: mod_base = OTHER; note = 2'-O-methyl uridine 3'-phosphodiester
  modified_base 12: mod_base = OTHER; note = 2'-O-methyl adenosine 3'-phosphodiester
  modified_base 13: mod_base = OTHER; note = 2'-O-methyl cytidine 3'-phosphodiester
  modified_base 14: mod_base = OTHER; note = 2'-deoxyuridine 3'-phosphodiester
  modified_base 15: mod_base = OTHER; note = 2'-O-methyl guanosine 3'-phosphodiester
  modified_base 16: mod_base = OTHER; note = 2'-deoxyuridine 3'-phosphodiester
  modified_base 17..19: mod_base = OTHER; note = 2'-O-methyl cytidine 3'-phosphodiester
  modified_base 20: mod_base = OTHER; note = 2'-O-methyl uridine 3'-phosphodiester
  modified_base 21: mod_base = OTHER; note = 2'-O-methyl uridine 3'-phosphorothioate
  modified_base 22: mod_base = OTHER; note = 2'-O-methoxyethyl adenosine 3'-phosphorothioate
  modified_base 23: mod_base = OTHER; note = 2'-O-methoxyethyl adenosine
tcactgagaatactgtcccttaa

SEQ ID NO: 26 - RNA, 23 nt, synthetic construct
  source 1..23: mol_type = other RNA
  modified_base 1: mod_base = OTHER; note = 2'-O-methoxyethyl thymidine 3'-phosphorothioate, conjugated at the 5' end to vinyl phosphonate
  modified_base 2: mod_base = OTHER; note = 2'-fluoro cytidine 3'-phosphorothioate
  modified_base 3: mod_base = OTHER; note = 2'-O-methyl adenosine 3'-phosphodiester
  modified_base 4: mod_base = OTHER; note = 2'-O-methyl cytidine 3'-phosphodiester
  modified_base 5: mod_base = OTHER; note = 2'-O-methyl uridine 3'-phosphodiester
  modified_base 6: mod_base = OTHER; note = 2'-deoxyguanosine 3'-phosphodiester
  modified_base 7: mod_base = OTHER; note = 2'-O-methyl adenosine 3'-phosphodiester
  modified_base 8: mod_base = OTHER; note = 2'-O-methyl guanosine 3'-phosphodiester
  modified_base 9..10: mod_base = OTHER; note = 2'-O-methoxyethyl adenosine 3'-phosphodiester
  modified_base 11: mod_base = OTHER; note = 2'-O-methyl uridine 3'-phosphodiester
  modified_base 12: mod_base = OTHER; note = 2'-O-methyl adenosine 3'-phosphodiester
  modified_base 13: mod_base = OTHER; note = 2'-O-methyl cytidine 3'-phosphodiester
  modified_base 14: mod_base = OTHER; note = 2'-deoxyuridine 3'-phosphodiester
  modified_base 15: mod_base = OTHER; note = 2'-O-methyl guanosine 3'-phosphodiester
  modified_base 16: mod_base = OTHER; note = 2'-deoxyuridine 3'-phosphodiester
  modified_base 17..19: mod_base = OTHER; note = 2'-O-methyl cytidine 3'-phosphodiester
  modified_base 20: mod_base = OTHER; note = 2'-O-methyl uridine 3'-phosphodiester
  modified_base 21: mod_base = OTHER; note = 2'-O-methyl uridine 3'-phosphorothioate
  modified_base 22: mod_base = OTHER; note = 2'-O-methoxyethyl adenosine 3'-phosphorothioate
  modified_base 23: mod_base = OTHER; note = 2'-O-methoxyethyl adenosine
tcactgagaatactgtcccttaa

SEQ ID NO: 27 - RNA, 23 nt, synthetic construct
  source 1..23: mol_type = other RNA
  modified_base 1: mod_base = OTHER; note = 2'-O-methoxyethyl thymidine 3'-phosphorothioate, conjugated at the 5' end to vinyl phosphonate
  modified_base 2: mod_base = OTHER; note = 2'-fluoro cytidine 3'-phosphorothioate
  modified_base 3: mod_base = OTHER; note = 2'-O-methyl adenosine 3'-phosphodiester
  modified_base 4: mod_base = OTHER; note = 2'-O-methyl cytidine 3'-phosphodiester
  modified_base 5: mod_base = OTHER; note = 2'-O-methyl uridine 3'-phosphodiester
  modified_base 6: mod_base = OTHER; note = 2'-deoxyguanosine 3'-phosphodiester
  modified_base 7: mod_base = OTHER; note = 2'-O-methyl adenosine 3'-phosphodiester
  modified_base 8: mod_base = OTHER; note = 2'-O-methyl guanosine 3'-phosphodiester
  modified_base 9: mod_base = OTHER; note = 2'-O-methoxyethyl adenosine 3'-phosphodiester
  modified_base 10: mod_base = OTHER; note = 2'-O-methyl adenosine 3'-phosphodiester
  modified_base 11: mod_base = OTHER; note = 2'-O-methyl uridine 3'-phosphodiester
  modified_base 12: mod_base = OTHER; note = 2'-O-methyl adenosine 3'-phosphodiester
  modified_base 13: mod_base = OTHER; note = 2'-O-methyl cytidine 3'-phosphodiester
  modified_base 14: mod_base = OTHER; note = 2'-fluoro uridine 3'-phosphodiester
  modified_base 15: mod_base = OTHER; note = 2'-O-methyl guanosine 3'-phosphodiester
  modified_base 16: mod_base = OTHER; note = 2'-fluoro uridine 3'-phosphodiester
  modified_base 17..19: mod_base = OTHER; note = 2'-O-methyl cytidine 3'-phosphodiester
  modified_base 20: mod_base = OTHER; note = 2'-O-methyl uridine 3'-phosphodiester
  modified_base 21: mod_base = OTHER; note = 2'-O-methyl uridine 3'-phosphorothioate
  modified_base 22: mod_base = OTHER; note = 2'-O-methoxyethyl adenosine 3'-phosphorothioate
  modified_base 23: mod_base = OTHER; note = 2'-O-methoxyethyl adenosine
tcactgagaatactgtcccttaa

SEQ ID NO: 28 - RNA, 23 nt, synthetic construct
  source 1..23: mol_type = other RNA
  modified_base 1: mod_base = OTHER; note = 2'-O-methoxyethyl thymidine 3'-phosphorothioate, conjugated at the 5' end to vinyl phosphonate
  modified_base 2: mod_base = OTHER; note = 2'-fluoro cytidine 3'-phosphorothioate
  modified_base 3: mod_base = OTHER; note = 2'-O-methyl adenosine 3'-phosphodiester
  modified_base 4: mod_base = OTHER; note = 2'-O-methyl cytidine 3'-phosphodiester
  modified_base 5: mod_base = OTHER; note = 2'-O-methyl uridine 3'-phosphodiester
  modified_base 6: mod_base = OTHER; note = 2'-deoxyguanosine 3'-phosphodiester
  modified_base 7: mod_base = OTHER; note = 2'-O-methyl adenosine 3'-phosphodiester
  modified_base 8: mod_base = OTHER; note = 2'-O-methyl guanosine 3'-phosphodiester
  modified_base 9: mod_base = OTHER; note = 2'-O-methoxyethyl adenosine 3'-phosphodiester
  modified_base 10: mod_base = OTHER; note = 2'-O-methyl adenosine 3'-phosphodiester
  modified_base 11: mod_base = OTHER; note = 2'-O-methyl uridine 3'-phosphodiester
  modified_base 12: mod_base = OTHER; note = 2'-O-methyl adenosine 3'-phosphodiester
  modified_base 13: mod_base = OTHER; note = 2'-O-methyl cytidine 3'-phosphodiester
  modified_base 14: mod_base = OTHER; note = 2'-deoxythymidine 3'-phosphodiester
  modified_base 15: mod_base = OTHER; note = 2'-O-methyl guanosine 3'-phosphodiester
  modified_base 16: mod_base = OTHER; note = 2'-deoxythymidine 3'-phosphodiester
  modified_base 17..19: mod_base = OTHER; note = 2'-O-methyl cytidine 3'-phosphodiester
  modified_base 20: mod_base = OTHER; note = 2'-O-methyl uridine 3'-phosphodiester
  modified_base 21: mod_base = OTHER; note = 2'-O-methyl uridine 3'-phosphorothioate
  modified_base 22: mod_base = OTHER; note = 2'-O-methoxyethyl adenosine 3'-phosphorothioate
  modified_base 23: mod_base = OTHER; note = 2'-O-methoxyethyl adenosine
tcactgagaatactgtcccttaa

SEQ ID NO: 29 - RNA, 23 nt, synthetic construct
  source 1..23: mol_type = other RNA
  modified_base 1: mod_base = OTHER; note = 2'-O-methoxyethyl thymidine 3'-phosphorothioate, conjugated at the 5' end to vinyl phosphonate
  modified_base 2: mod_base = OTHER; note = 2'-fluoro cytidine 3'-phosphorothioate
  modified_base 3: mod_base = OTHER; note = 2'-O-methyl adenosine 3'-phosphodiester
  modified_base 4: mod_base = OTHER; note = 2'-O-methyl cytidine 3'-phosphodiester
  modified_base 5: mod_base = OTHER; note = 2'-O-methyl uridine 3'-phosphodiester
  modified_base 6: mod_base = OTHER; note = 2'-deoxyguanosine 3'-phosphorothioate
  modified_base 7: mod_base = OTHER; note = 2'-O-methyl adenosine 3'-phosphodiester
  modified_base 8: mod_base = OTHER; note = 2'-O-methyl guanosine 3'-phosphodiester
  modified_base 9: mod_base = OTHER; note = 2'-O-methoxyethyl adenosine 3'-phosphodiester
  modified_base 10: mod_base = OTHER; note = 2'-O-methyl adenosine 3'-phosphodiester
  modified_base 11: mod_base = OTHER; note = 2'-O-methyl uridine 3'-phosphodiester
  modified_base 12: mod_base = OTHER; note = 2'-O-methyl adenosine 3'-phosphodiester
  modified_base 13: mod_base = OTHER; note = 2'-O-methyl cytidine 3'-phosphodiester
  modified_base 14: mod_base = OTHER; note = 2'-deoxyuridine 3'-phosphorothioate
  modified_base 15: mod_base = OTHER; note = 2'-O-methyl guanosine 3'-phosphodiester
  modified_base 16: mod_base = OTHER; note = 2'-deoxyuridine 3'-phosphorothioate
  modified_base 17..19: mod_base = OTHER; note = 2'-O-methyl cytidine 3'-phosphodiester
  modified_base 20: mod_base = OTHER; note = 2'-O-methyl uridine 3'-phosphodiester
  modified_base 21: mod_base = OTHER; note = 2'-O-methyl uridine 3'-phosphorothioate
  modified_base 22: mod_base = OTHER; note = 2'-O-methoxyethyl adenosine 3'-phosphorothioate
  modified_base 23: mod_base = OTHER; note = 2'-O-methoxyethyl adenosine
tcactgagaatactgtcccttaa

SEQ ID NO: 30 - RNA, 23 nt, synthetic construct
  source 1..23: mol_type = other RNA
  modified_base 1: mod_base = OTHER; note = 2'-O-methoxyethyl thymidine 3'-phosphorothioate, conjugated at the 5' end to vinyl phosphonate
  modified_base 2: mod_base = OTHER; note = 2'-fluoro cytidine 3'-phosphorothioate
  modified_base 3: mod_base = OTHER; note = 2'-O-methyl adenosine 3'-phosphodiester
  modified_base 4: mod_base = OTHER; note = 2'-O-methyl cytidine 3'-phosphodiester
  modified_base 5: mod_base = OTHER; note = 2'-O-methyl uridine 3'-phosphodiester
  modified_base 6: mod_base = OTHER; note = 2'-deoxyguanosine 3'-phosphodiester
  modified_base 7: mod_base = OTHER; note = 2'-O-methyl adenosine 3'-phosphodiester
  modified_base 8: mod_base = OTHER; note = 2'-O-methyl guanosine 3'-phosphodiester
  modified_base 9..10: mod_base = OTHER; note = 2'-O-methoxyethyl adenosine 3'-phosphodiester
  modified_base 11: mod_base = OTHER; note = 2'-O-methyl uridine 3'-phosphodiester
  modified_base 12: mod_base = OTHER; note = 2'-O-methyl adenosine 3'-phosphodiester
  modified_base 13: mod_base = OTHER; note = 2'-O-methyl cytidine 3'-phosphodiester
  modified_base 14: mod_base = OTHER; note = 2'-fluoro uridine 3'-phosphodiester
  modified_base 15: mod_base = OTHER; note = 2'-O-methyl guanosine 3'-phosphodiester
  modified_base 16: mod_base = OTHER; note = 2'-fluoro uridine 3'-phosphodiester
  modified_base 17..19: mod_base = OTHER; note = 2'-O-methyl cytidine 3'-phosphodiester
  modified_base 20: mod_base = OTHER; note = 2'-O-methyl uridine 3'-phosphodiester
  modified_base 21: mod_base = OTHER; note = 2'-O-methyl uridine 3'-phosphorothioate
  modified_base 22: mod_base = OTHER; note = 2'-O-methoxyethyl adenosine 3'-phosphorothioate
  modified_base 23: mod_base = OTHER; note = 2'-O-methoxyethyl adenosine
tcactgagaatactgtcccttaa

SEQ ID NO: 31 - RNA, 23 nt, synthetic construct
  source 1..23: mol_type = other RNA
  modified_base 1: mod_base = OTHER; note = 2'-O-methoxyethyl thymidine 3'-phosphorothioate, conjugated at the 5' end to vinyl phosphonate
  modified_base 2: mod_base = OTHER; note = 2'-fluoro cytidine 3'-phosphorothioate
  modified_base 3: mod_base = OTHER; note = 2'-O-methyl adenosine 3'-phosphodiester
  modified_base 4: mod_base = OTHER; note = 2'-O-methyl cytidine 3'-phosphodiester
  modified_base 5: mod_base = OTHER; note = 2'-O-methyl uridine 3'-phosphodiester
  modified_base 6: mod_base = OTHER; note = 2'-deoxyguanosine 3'-phosphorothioate
  modified_base 7: mod_base = OTHER; note = 2'-O-methyl adenosine 3'-phosphodiester
  modified_base 8: mod_base = OTHER; note = 2'-O-methyl guanosine 3'-phosphodiester
  modified_base 9: mod_base = OTHER; note = 2'-O-methoxyethyl adenosine 3'-phosphodiester
  modified_base 10: mod_base = OTHER; note = 2'-O-methyl adenosine 3'-phosphodiester
  modified_base 11: mod_base = OTHER; note = 2'-O-methyl uridine 3'-phosphodiester
  modified_base 12: mod_base = OTHER; note = 2'-O-methyl adenosine 3'-phosphodiester
  modified_base 13: mod_base = OTHER; note = 2'-O-methyl cytidine 3'-phosphodiester
  modified_base 14: mod_base = OTHER; note = 2'-deoxythymidine 3'-phosphorothioate
  modified_base 15: mod_base = OTHER; note = 2'-O-methyl guanosine 3'-phosphodiester
  modified_base 16: mod_base = OTHER; note = 2'-deoxythymidine 3'-phosphorothioate
  modified_base 17..19: mod_base = OTHER; note = 2'-O-methyl cytidine 3'-phosphodiester
  modified_base 20: mod_base = OTHER; note = 2'-O-methyl uridine 3'-phosphodiester
  modified_base 21: mod_base = OTHER; note = 2'-O-methyl uridine 3'-phosphorothioate
  modified_base 22: mod_base = OTHER; note = 2'-O-methoxyethyl adenosine 3'-phosphorothioate
  modified_base 23: mod_base = OTHER; note = 2'-O-methoxyethyl adenosine
tcactgagaatactgtcccttaa

SEQ ID NO: 32 - RNA, 21 nt, synthetic construct
  source 1..21: mol_type = other RNA
  modified_base 1: mod_base = OTHER; note = 2'-O-methoxyethyl thymidine 3'-phosphorothioate, conjugated at the 5' end to vinyl phosphonate
  modified_base 2: mod_base = OTHER; note = 2'-fluoro cytidine 3'-phosphorothioate
  modified_base 3: mod_base = OTHER; note = 2'-O-methyl adenosine 3'-phosphodiester
  modified_base 4: mod_base = OTHER; note = 2'-O-methyl cytidine 3'-phosphodiester
  modified_base 5: mod_base = OTHER; note = 2'-O-methyl uridine 3'-phosphodiester
  modified_base 6: mod_base = OTHER; note = 2'-fluoro guanosine 3'-phosphodiester
  modified_base 7: mod_base = OTHER; note = 2'-O-methyl adenosine 3'-phosphodiester
  modified_base 8: mod_base = OTHER; note = 2'-O-methyl guanosine 3'-phosphodiester
  modified_base 9..10: mod_base = OTHER; note = 2'-O-methyl adenosine 3'-phosphodiester
  modified_base 11: mod_base = OTHER; note = 2'-O-methyl uridine 3'-phosphodiester
  modified_base 12: mod_base = OTHER; note = 2'-O-methyl adenosine 3'-phosphodiester
  modified_base 13: mod_base = OTHER; note = 2'-O-methyl cytidine 3'-phosphodiester
  modified_base 14: mod_base = OTHER; note = 2'-fluoro uridine 3'-phosphodiester
  modified_base 15: mod_base = OTHER; note = 2'-O-methyl guanosine 3'-phosphodiester
  modified_base 16: mod_base = OTHER; note = 2'-fluoro uridine 3'-phosphodiester
  modified_base 17..18: mod_base = OTHER; note = 2'-O-methyl cytidine 3'-phosphodiester
  modified_base 19: mod_base = OTHER; note = 2'-O-methyl cytidine 3'-phosphorothioate
  modified_base 20: mod_base = OTHER; note = 2'-O-methoxyethyl adenosine 3'-phosphorothioate
  modified_base 21: mod_base = OTHER; note = 2'-O-methoxyethyl adenosine
tcactgagaatactgtcccaa

SEQ ID NO: 33 - RNA, 21 nt, synthetic construct
  source 1..21: mol_type = other RNA
  modified_base 1: mod_base = OTHER; note = 2'-O-methoxyethyl thymidine 3'-phosphorothioate, conjugated at the 5' end to vinyl phosphonate
  modified_base 2: mod_base = OTHER; note = 2'-fluoro cytidine 3'-phosphorothioate
  modified_base 3: mod_base = OTHER; note = 2'-O-methyl adenosine 3'-phosphodiester
  modified_base 4: mod_base = OTHER; note = 2'-O-methyl cytidine 3'-phosphodiester
  modified_base 5: mod_base = OTHER; note = 2'-O-methyl uridine 3'-phosphodiester
  modified_base 6: mod_base = OTHER; note = 2'-deoxyguanosine 3'-phosphorothioate
  modified_base 7: mod_base = OTHER; note = 2'-O-methyl adenosine 3'-phosphodiester
  modified_base 8: mod_base = OTHER; note = 2'-O-methyl guanosine 3'-phosphodiester
  modified_base 9..10: mod_base = OTHER; note = 2'-O-methyl adenosine 3'-phosphodiester
  modified_base 11: mod_base = OTHER; note = 2'-O-methyl uridine 3'-phosphodiester
  modified_base 12: mod_base = OTHER; note = 2'-O-methyl adenosine 3'-phosphodiester
  modified_base 13: mod_base = OTHER; note = 2'-O-methyl cytidine 3'-phosphodiester
  modified_base 14: mod_base = OTHER; note = 2'-fluoro uridine 3'-phosphodiester
  modified_base 15: mod_base = OTHER; note = 2'-O-methyl guanosine 3'-phosphodiester
  modified_base 16: mod_base = OTHER; note = 2'-fluoro uridine 3'-phosphodiester
  modified_base 17..18: mod_base = OTHER; note = 2'-O-methyl cytidine 3'-phosphodiester
  modified_base 19: mod_base = OTHER; note = 2'-O-methyl cytidine 3'-phosphorothioate
  modified_base 20: mod_base = OTHER; note = 2'-O-methoxyethyl adenosine 3'-phosphorothioate
  modified_base 21: mod_base = OTHER; note = 2'-O-methoxyethyl adenosine
tcactgagaatactgtcccaa

SEQ ID NO: 34 - RNA, 21 nt, synthetic construct
  source 1..21: mol_type = other RNA
  modified_base 1: mod_base = OTHER; note = 2'-O-methoxyethyl thymidine 3'-phosphorothioate, conjugated at the 5' end to vinyl phosphonate
  modified_base 2: mod_base = OTHER; note = 2'-fluoro cytidine 3'-phosphorothioate
  modified_base 3: mod_base = OTHER; note = 2'-O-methyl adenosine 3'-phosphodiester
  modified_base 4: mod_base = OTHER; note = 2'-O-methyl cytidine 3'-phosphodiester
  modified_base 5: mod_base = OTHER; note = 2'-O-methyl uridine 3'-phosphodiester
  modified_base 6: mod_base = OTHER; note = 2'-deoxyguanosine 3'-phosphorothioate
  modified_base 7: mod_base = OTHER; note = 2'-O-methyl adenosine 3'-phosphodiester
  modified_base 8: mod_base = OTHER; note = 2'-O-methyl guanosine 3'-phosphodiester
  modified_base 9..10: mod_base = OTHER; note = 2'-O-methyl adenosine 3'-phosphodiester
  modified_base 11: mod_base = OTHER; note = 2'-O-methyl uridine 3'-phosphodiester
  modified_base 12: mod_base = OTHER; note = 2'-O-methyl adenosine 3'-phosphodiester
  modified_base 13: mod_base = OTHER; note = 2'-O-methyl cytidine 3'-phosphodiester
  modified_base 14: mod_base = OTHER; note = 2'-deoxyuridine 3'-phosphorothioate
  modified_base 15: mod_base = OTHER; note = 2'-O-methyl guanosine 3'-phosphodiester
  modified_base 16: mod_base = OTHER; note = 2'-deoxyuridine 3'-phosphorothioate
  modified_base 17..18: mod_base = OTHER; note = 2'-O-methyl cytidine 3'-phosphodiester
  modified_base 19: mod_base = OTHER; note = 2'-O-methyl cytidine 3'-phosphorothioate
  modified_base 20: mod_base = OTHER; note = 2'-O-methoxyethyl adenosine 3'-phosphorothioate
  modified_base 21: mod_base = OTHER; note = 2'-O-methoxyethyl adenosine
tcactgagaatactgtcccaa

SEQ ID NO: 35 - RNA, 21 nt, synthetic construct
  source 1..21: mol_type = other RNA
  modified_base 2: mod_base = OTHER; note = 2'-O-methyl adenosine 3'-phosphorothioate
  modified_base 3..5: mod_base = OTHER; note = 2'-O-methyl guanosine 3'-phosphodiester
  modified_base 6: mod_base = OTHER; note = 2'-O-methyl adenosine 3'-phosphodiester
  modified_base 7: mod_base = OTHER; note = 2'-fluoro cytidine 3'-phosphodiester
  modified_base 8: mod_base = OTHER; note = 2'-O-methyl adenosine 3'-phosphodiester
  modified_base 9: mod_base = OTHER; note = 2'-fluoro guanosine 3'-phosphodiester
  modified_base 10: mod_base = OTHER; note = 2'-fluoro uridine 3'-phosphodiester
  modified_base 11: mod_base = OTHER; note = 2'-fluoro adenosine 3'-phosphodiester
  modified_base 12..13: mod_base = OTHER; note = 2'-O-methyl uridine 3'-phosphodiester
  modified_base 14: mod_base = OTHER; note = 2'-O-methyl cytidine 3'-phosphodiester
  modified_base 15: mod_base = OTHER; note = 2'-O-methyl uridine 3'-phosphodiester
  modified_base 16: mod_base = OTHER; note = 2'-O-methyl cytidine 3'-phosphodiester
  modified_base 17: mod_base = OTHER; note = 2'-O-methyl adenosine 3'-phosphodiester
  modified_base 18: mod_base = OTHER; note = 2'-O-methyl guanosine 3'-phosphodiester
  modified_base 19: mod_base = OTHER; note = 2'-O-methyl uridine 3'-phosphorothioate
  modified_base 20: mod_base = OTHER; note = 2'-O-methyl guanosine 3'-phosphorothioate
  modified_base 21: mod_base = OTHER; note = 2'-O-methyl adenosine
  modified_base 1: mod_base = OTHER; note = 2'-O-methyl adenosine 3'-phosphorothioate, conjugated at the 5' end to trishexylamino-N-Acetylgalactosamine-6-aminohexyl phosphodiester conjugate
aagggacagtattctcagtga

SEQ ID NO: 36 - RNA, 21 nt, synthetic construct
  source 1..21: mol_type = other RNA
  modified_base 2: mod_base = OTHER; note = 2'-O-methyl adenosine 3'-phosphorothioate
  modified_base 3..5: mod_base = OTHER; note = 2'-O-methyl guanosine 3'-phosphodiester
  modified_base 6: mod_base = OTHER; note = 2'-O-methyl adenosine 3'-phosphodiester
  modified_base 7: mod_base = OTHER; note = 2'-O-methyl cytidine 3'-phosphodiester
  modified_base 8: mod_base = OTHER; note = 2'-O-methyl adenosine 3'-phosphodiester
  modified_base 9: mod_base = OTHER; note = 2'-O-methyl guanosine 3'-phosphodiester
  modified_base 10: mod_base = OTHER; note = 2'-fluoro uridine 3'-phosphodiester
  modified_base 11: mod_base = OTHER; note = 2'-fluoro adenosine 3'-phosphodiester
  modified_base 12..13: mod_base = OTHER; note = 2'-O-methyl uridine 3'-phosphodiester
  modified_base 14: mod_base = OTHER; note = 2'-O-methyl cytidine 3'-phosphodiester
  modified_base 15: mod_base = OTHER; note = 2'-O-methyl uridine 3'-phosphodiester
  modified_base 16: mod_base = OTHER; note = 2'-O-methyl cytidine 3'-phosphodiester
  modified_base 17: mod_base = OTHER; note = 2'-O-methyl adenosine 3'-phosphodiester
  modified_base 18: mod_base = OTHER; note = 2'-O-methyl guanosine 3'-phosphodiester
  modified_base 19: mod_base = OTHER; note = 2'-O-methyl uridine 3'-phosphorothioate
  modified_base 20: mod_base = OTHER; note = 2'-O-methyl guanosine 3'-phosphorothioate
  modified_base 21: mod_base = OTHER; note = 2'-O-methyl adenosine
  modified_base 1: mod_base = OTHER; note = 2'-O-methyl adenosine 3'-phosphorothioate, conjugated at the 5' end to trishexylamino-N-Acetylgalactosamine-6-aminohexyl phosphodiester conjugate
aagggacagtattctcagtga

SEQ ID NO: 37 - RNA, 21 nt, synthetic construct
  source 1..21: mol_type = other RNA
  modified_base 2: mod_base = OTHER; note = 2'-O-methyl adenosine 3'-phosphorothioate
  modified_base 3..5: mod_base = OTHER; note = 2'-O-methyl guanosine 3'-phosphodiester
  modified_base 6: mod_base = OTHER; note = 2'-O-methyl adenosine 3'-phosphodiester
  modified_base 7: mod_base = OTHER; note = 2'-O-methyl cytidine 3'-phosphodiester
  modified_base 8: mod_base = OTHER; note = 2'-fluoro adenosine 3'-phosphodiester
  modified_base 9: mod_base = OTHER; note = 2'-O-methyl guanosine 3'-phosphodiester
  modified_base 10: mod_base = OTHER; note = 2'-fluoro uridine 3'-phosphodiester
  modified_base 11: mod_base = OTHER; note = 2'-O-methyl adenosine 3'-phosphodiester
  modified_base 12: mod_base = OTHER; note = 2'-fluoro uridine 3'-phosphodiester
  modified_base 13: mod_base = OTHER; note = 2'-O-methyl uridine 3'-phosphodiester
  modified_base 14: mod_base = OTHER; note = 2'-O-methyl cytidine 3'-phosphodiester
  modified_base 15: mod_base = OTHER; note = 2'-O-methyl uridine 3'-phosphodiester
  modified_base 16: mod_base = OTHER; note = 2'-O-methyl cytidine 3'-phosphodiester
  modified_base 17: mod_base = OTHER; note = 2'-O-methyl adenosine 3'-phosphodiester
  modified_base 18: mod_base = OTHER; note = 2'-O-methyl guanosine 3'-phosphodiester
  modified_base 19: mod_base = OTHER; note = 2'-O-methyl uridine 3'-phosphorothioate
  modified_base 20: mod_base = OTHER; note = 2'-O-methyl guanosine 3'-phosphorothioate
  modified_base 21: mod_base = OTHER; note = 2'-O-methyl adenosine
  modified_base 1: mod_base = OTHER; note = 2'-O-methyl adenosine 3'-phosphorothioate, conjugated at the 5' end to trishexylamino-N-Acetylgalactosamine-6-aminohexyl phosphodiester conjugate
aagggacagtattctcagtga

SEQ ID NO: 38 - RNA, 21 nt, synthetic construct
  source 1..21: mol_type = other RNA
  modified_base 2: mod_base = OTHER; note = 2'-O-methyl adenosine 3'-phosphorothioate
  modified_base 3..5: mod_base = OTHER; note = 2'-O-methyl guanosine 3'-phosphodiester
  modified_base 6: mod_base = OTHER; note = 2'-O-methyl adenosine 3'-phosphodiester
  modified_base 8: mod_base = OTHER; note = 2'-O-methyl adenosine 3'-phosphodiester
  modified_base 7: mod_base = OTHER; note = 2'-O-methyl cytidine 3'-phosphodiester
  modified_base 9: mod_base = OTHER; note = 2'-O-methyl guanosine 3'-phosphodiester
  modified_base 10: mod_base = OTHER; note = 2'-fluoro uridine 3'-phosphorothioate
  modified_base 11: mod_base = OTHER; note = 2'-fluoro adenosine 3'-phosphodiester
  modified_base 12..13: mod_base = OTHER; note = 2'-O-methyl uridine 3'-phosphodiester
  modified_base 15: mod_base = OTHER; note = 2'-O-methyl uridine 3'-phosphodiester
  modified_base 14: mod_base = OTHER; note = 2'-O-methyl cytidine 3'-phosphodiester
  modified_base 16: mod_base = OTHER; note = 2'-O-methyl cytidine 3'-phosphodiester
  modified_base 17: mod_base = OTHER; note = 2'-O-methyl adenosine 3'-phosphodiester
  modified_base 18: mod_base = OTHER; note = 2'-O-methyl guanosine 3'-phosphodiester
  modified_base 19: mod_base = OTHER; note = 2'-O-methyl uridine 3'-phosphorothioate
  modified_base 20: mod_base = OTHER; note = 2'-O-methyl guanosine 3'-phosphorothioate
  modified_base 21: mod_base = OTHER; note = 2'-O-methyl adenosine
  modified_base 1: mod_base = OTHER; note = 2'-O-methyl adenosine 3'-phosphorothioate, conjugated at the 5' end to trishexylamino-N-Acetylgalactosamine-6-aminohexyl phosphodiester conjugate
aagggacagtattctcagtga

SEQ ID NO: 39 - RNA, 21 nt, synthetic construct
  source 1..21: mol_type = other RNA
  modified_base 2: mod_base = OTHER; note = 2'-O-methoxyethyl adenosine 3'-phosphorothioate
  modified_base 3..5: mod_base = OTHER; note = 2'-O-methyl guanosine 3'-phosphodiester
  modified_base 6: mod_base = OTHER; note = 2'-O-methyl adenosine 3'-phosphodiester
  modified_base 7: mod_base = OTHER; note = 2'-O-methyl cytidine 3'-phosphodiester
  modified_base 8: mod_base = OTHER; note = 2'-O-methyl adenosine 3'-phosphodiester
  modified_base 9: mod_base = OTHER; note = 2'-O-methyl guanosine 3'-phosphodiester
  modified_base 10: mod_base = OTHER; note = 2'-fluoro uridine 3'-phosphodiester
  modified_base 11: mod_base = OTHER; note = 2'-fluoro adenosine 3'-phosphodiester
  modified_base 12..13: mod_base = OTHER; note = 2'-O-methyl uridine 3'-phosphodiester
  modified_base 14: mod_base = OTHER; note = 2'-O-methyl cytidine 3'-phosphodiester
  modified_base 15: mod_base = OTHER; note = 2'-O-methyl uridine 3'-phosphodiester
  modified_base 16: mod_base = OTHER; note = 2'-O-methyl cytidine 3'-phosphodiester
  modified_base 17: mod_base = OTHER; note = 2'-O-methyl adenosine 3'-phosphodiester
  modified_base 18: mod_base = OTHER; note = 2'-O-methyl guanosine 3'-phosphodiester
  modified_base 19: mod_base = OTHER; note = 2'-O-methyl uridine 3'-phosphorothioate
  modified_base 20: mod_base = OTHER; note = 2'-O-methoxyethyl guanosine 3'-phosphorothioate
  modified_base 21: mod_base = OTHER; note = 2'-O-methoxyethyl adenosine
  modified_base 1: mod_base = OTHER; note = 2'-O-methoxyethyl adenosine 3'-phosphorothioate, conjugated at the 5' end to trishexylamino-N-Acetylgalactosamine-6-aminohexyl phosphodiester conjugate
aagggacagtattctcagtga

SEQ ID NO: 40 - RNA, 21 nt, synthetic construct
  source 1..21: mol_type = other RNA
  modified_base 2: mod_base = OTHER; note = 2'-O-methoxyethyl adenosine 3'-phosphorothioate
  modified_base 3..5: mod_base = OTHER; note = 2'-O-methyl guanosine 3'-phosphodiester
  modified_base 6: mod_base = OTHER; note = 2'-O-methyl adenosine 3'-phosphodiester
  modified_base 7: mod_base = OTHER; note = 2'-O-methyl cytidine 3'-phosphodiester
  modified_base 8: mod_base = OTHER; note = 2'-O-methyl adenosine 3'-phosphodiester
  modified_base 9: mod_base = OTHER; note = 2'-O-methyl guanosine 3'-phosphodiester
  modified_base 10: mod_base = OTHER; note = 2'-fluoro uridine 3'-phosphorothioate
  modified_base 11: mod_base = OTHER; note = 2'-fluoro adenosine 3'-phosphodiester
  modified_base 12..13: mod_base = OTHER; note = 2'-O-methyl uridine 3'-phosphodiester
  modified_base 14: mod_base = OTHER; note = 2'-O-methyl cytidine 3'-phosphodiester
  modified_base 15: mod_base = OTHER; note = 2'-O-methyl uridine 3'-phosphodiester
  modified_base 16: mod_base = OTHER; note = 2'-O-methyl cytidine 3'-phosphodiester
  modified_base 17: mod_base = OTHER; note = 2'-O-methyl adenosine 3'-phosphodiester
  modified_base 18: mod_base = OTHER; note = 2'-O-methyl guanosine 3'-phosphodiester
  modified_base 19: mod_base = OTHER; note = 2'-O-methyl uridine 3'-phosphorothioate
  modified_base 20: mod_base = OTHER; note = 2'-O-methoxyethyl guanosine 3'-phosphorothioate
  modified_base 21: mod_base = OTHER; note = 2'-O-methoxyethyl adenosine
  modified_base 1: mod_base = OTHER; note = 2'-O-methoxyethyl adenosine 3'-phosphorothioate, conjugated at the 5' end to trishexylamino-N-Acetylgalactosamine-6-aminohexyl phosphodiester conjugate
aagggacagtattctcagtga

SEQ ID NO: 41 - RNA, 21 nt, synthetic construct
  source 1..21: mol_type = other RNA
  modified_base 2: mod_base = OTHER; note = 2'-O-methoxyethyl adenosine 3'-phosphorothioate
  modified_base 3..5: mod_base = OTHER; note = 2'-O-methyl guanosine 3'-phosphodiester
  modified_base 6: mod_base = OTHER; note = 2'-O-methyl adenosine 3'-phosphodiester
  modified_base 7: mod_base = OTHER; note = 2'-O-methyl cytidine 3'-phosphodiester
  modified_base 8: mod_base = OTHER; note = 2'-O-methyl adenosine 3'-phosphodiester
  modified_base 9: mod_base = OTHER; note = 2'-fluoro guanosine 3'-phosphorothioate
  modified_base 10: mod_base = OTHER; note = 2'-fluoro uridine 3'-phosphodiester
  modified_base 11: mod_base = OTHER; note = 2'-O-methyl adenosine 3'-phosphodiester
  modified_base 12..13: mod_base = OTHER; note = 2'-O-methyl uridine 3'-phosphodiester
  modified_base 14: mod_base = OTHER; note = 2'-O-methyl cytidine 3'-phosphodiester
  modified_base 15: mod_base = OTHER; note = 2'-O-methyl uridine 3'-phosphodiester
  modified_base 16: mod_base = OTHER; note = 2'-O-methyl cytidine 3'-phosphodiester
  modified_base 17: mod_base = OTHER; note = 2'-O-methyl adenosine 3'-phosphodiester
  modified_base 18: mod_base = OTHER; note = 2'-O-methyl guanosine 3'-phosphodiester
  modified_base 19: mod_base = OTHER; note = 2'-O-methyl uridine 3'-phosphorothioate
  modified_base 20: mod_base = OTHER; note = 2'-O-methoxyethyl guanosine 3'-phosphorothioate
  modified_base 21: mod_base = OTHER; note = 2'-O-methoxyethyl adenosine
  modified_base 1: mod_base = OTHER; note = 2'-O-methoxyethyl adenosine 3'-phosphorothioate, conjugated at the 5' end to trishexylamino-N-Acetylgalactosamine-6-aminohexyl phosphodiester conjugate
aagggacagtattctcagtga

SEQ ID NO: 42 - RNA, 19 nt, synthetic construct
  source 1..19: mol_type = other RNA
  modified_base 2: mod_base = OTHER; note = 2'-O-methyl guanosine 3'-phosphorothioate
  modified_base 3: mod_base = OTHER; note = 2'-O-methyl guanosine 3'-phosphodiester
  modified_base 4: mod_base = OTHER; note = 2'-O-methyl adenosine 3'-phosphodiester
  modified_base 5: mod_base = OTHER; note = 2'-O-methyl cytidine 3'-phosphodiester
  modified_base 6: mod_base = OTHER; note = 2'-O-methyl adenosine 3'-phosphodiester
  modified_base 7: mod_base = OTHER; note = 2'-fluoro guanosine 3'-phosphodiester
  modified_base 8: mod_base = OTHER; note = 2'-O-methyl uridine 3'-phosphodiester
  modified_base 9: mod_base = OTHER; note = 2'-fluoro adenosine 3'-phosphodiester
  modified_base 10..11: mod_base = OTHER; note = 2'-fluoro uridine 3'-phosphodiester
  modified_base 12: mod_base = OTHER; note = 2'-O-methyl cytidine 3'-phosphodiester
  modified_base 13: mod_base = OTHER; note = 2'-O-methyl uridine 3'-phosphodiester
  modified_base 14: mod_base = OTHER; note = 2'-O-methyl cytidine 3'-phosphodiester
  modified_base 15: mod_base = OTHER; note = 2'-O-methyl adenosine 3'-phosphodiester
  modified_base 16: mod_base = OTHER; note = 2'-O-methyl guanosine 3'-phosphodiester
  modified_base 17: mod_base = OTHER; note = 2'-O-methyl uridine 3'-phosphorothioate
  modified_base 18: mod_base = OTHER; note = 2'-O-methyl guanosine 3'-phosphorothioate
  modified_base 19: mod_base = OTHER; note = 2'-O-methyl adenosine
  modified_base 1: mod_base = OTHER; note = 2'-O-methyl guanosine 3'-phosphorothioate, conjugated at the 5' end to trishexylamino-N-Acetylgalactosamine-6-aminohexyl phosphodiester conjugate
gggacagtattctcagtga

SEQ ID NO: 43 - RNA, 19 nt, synthetic construct
  source 1..19: mol_type = other RNA
  modified_base 2: mod_base = OTHER; note = 2'-O-methyl guanosine 3'-phosphorothioate
  modified_base 3: mod_base = OTHER; note = 2'-O-methyl guanosine 3'-phosphodiester
  modified_base 4: mod_base = OTHER; note = 2'-O-methyl adenosine 3'-phosphodiester
  modified_base 5: mod_base = OTHER; note = 2'-O-methyl cytidine 3'-phosphodiester
  modified_base 6: mod_base = OTHER; note = 2'-O-methyl adenosine 3'-phosphodiester
  modified_base 7: mod_base = OTHER; note = 2'-fluoro guanosine 3'-phosphodiester
  modified_base 8: mod_base = OTHER; note = 2'-O-methyl uridine 3'-phosphodiester
  modified_base 9: mod_base = OTHER; note = 2'-fluoro adenosine 3'-phosphodiester
  modified_base 10..11: mod_base = OTHER; note = 2'-O-methyl uridine 3'-phosphodiester
  modified_base 12: mod_base = OTHER; note = 2'-O-methyl cytidine 3'-phosphodiester
  modified_base 13: mod_base = OTHER; note = 2'-O-methyl uridine 3'-phosphodiester
  modified_base 14: mod_base = OTHER; note = 2'-O-methyl cytidine 3'-phosphodiester
  modified_base 15: mod_base = OTHER; note = 2'-O-methyl adenosine 3'-phosphodiester
  modified_base 16: mod_base = OTHER; note = 2'-O-methyl guanosine 3'-phosphodiester
  modified_base 17: mod_base = OTHER; note = 2'-O-methyl uridine 3'-phosphorothioate
  modified_base 18: mod_base = OTHER; note = 2'-O-methyl guanosine 3'-phosphorothioate
  modified_base 19: mod_base = OTHER; note = 2'-O-methyl adenosine
  modified_base 1: mod_base = OTHER; note = 2'-O-methyl guanosine 3'-phosphorothioate, conjugated at the 5' end to trishexylamino-N-Acetylgalactosamine-6-aminohexyl phosphodiester conjugate
gggacagtattctcagtga

SEQ ID NO: 44 - RNA, 19 nt, synthetic construct
  source 1..19: mol_type = other RNA
  modified_base 2: mod_base = OTHER; note = 2'-O-methyl guanosine 3'-phosphorothioate
  modified_base 3: mod_base = OTHER; note = 2'-O-methyl guanosine 3'-phosphodiester
  modified_base 4: mod_base = OTHER; note = 2'-O-methyl adenosine 3'-phosphodiester
  modified_base 6: mod_base = OTHER; note = 2'-O-methyl adenosine 3'-phosphodiester
  modified_base 5: mod_base = OTHER; note = 2'-O-methyl cytidine 3'-phosphodiester
  modified_base 7: mod_base = OTHER; note = 2'-O-methyl guanosine 3'-phosphodiester
  modified_base 8: mod_base = OTHER; note = 2'-fluoro uridine 3'-phosphodiester
  modified_base 9: mod_base = OTHER; note = 2'-O-methyl adenosine 3'-phosphodiester
  modified_base 10: mod_base = OTHER; note = 2'-fluoro uridine 3'-phosphodiester
  modified_base 11: mod_base = OTHER; note = 2'-O-methyl uridine 3'-phosphodiester
  modified_base 12: mod_base = OTHER; note = 2'-O-methyl cytidine 3'-phosphodiester
  modified_base 13: mod_base = OTHER; note = 2'-O-methyl uridine 3'-phosphodiester
  modified_base 14: mod_base = OTHER; note = 2'-O-methyl cytidine 3'-phosphodiester
  modified_base 16: mod_base = OTHER; note = 2'-O-methyl adenosine 3'-phosphodiester
  modified_base 16: mod_base = OTHER; note = 2'-O-methyl guanosine 3'-phosphodiester
  modified_base 17: mod_base = OTHER; note = 2'-O-methyl uridine 3'-phosphorothioate
  modified_base 18: mod_base = OTHER; note = 2'-O-methyl guanosine 3'-phosphorothioate
  modified_base 19: mod_base = OTHER; note = 2'-O-methyl adenosine
  modified_base 1: mod_base = OTHER; note = 2'-O-methyl guanosine 3'-phosphorothioate, conjugated at the 5' end to trishexylamino-N-Acetylgalactosamine-6-aminohexyl phosphodiester conjugate
gggacagtattctcagtga

SEQ ID NO: 45 - RNA, 19 nt, synthetic construct
  source 1..19: mol_type = other RNA
  modified_base 2: mod_base = OTHER; note = 2'-O-methyl guanosine 3'-phosphorothioate
  modified_base 3: mod_base = OTHER; note = 2'-O-methyl guanosine 3'-phosphodiester
  modified_base 4: mod_base = OTHER; note = 2'-O-methyl adenosine 3'-phosphodiester
  modified_base 5: mod_base = OTHER; note = 2'-O-methyl cytidine 3'-phosphodiester
  modified_base 6: mod_base = OTHER; note = 2'-O-methyl adenosine 3'-phosphodiester
  modified_base 7: mod_base = OTHER; note = 2'-O-methyl guanosine 3'-phosphodiester
  modified_base 8: mod_base = OTHER; note = 2'-O-methyl uridine 3'-phosphodiester
  modified_base 9: mod_base = OTHER; note = 2'-fluoro adenosine 3'-phosphodiester
  modified_base 10: mod_base = OTHER; note = 2'-O-methyl uridine 3'-phosphodiester
  modified_base 11: mod_base = OTHER; note = 2'-fluoro uridine 3'-phosphodiester
  modified_base 12: mod_base = OTHER; note = 2'-O-methyl cytidine 3'-phosphodiester
  modified_base 13: mod_base = OTHER; note = 2'-O-methyl uridine 3'-phosphodiester
  modified_base 14: mod_base = OTHER; note = 2'-O-methyl cytidine 3'-phosphodiester
  modified_base 15: mod_base = OTHER; note = 2'-O-methyl adenosine 3'-phosphodiester
  modified_base 16: mod_base = OTHER; note = 2'-O-methyl guanosine 3'-phosphodiester
  modified_base 17: mod_base = OTHER; note = 2'-O-methyl uridine 3'-phosphorothioate
  modified_base 18: mod_base = OTHER; note = 2'-O-methyl guanosine 3'-phosphorothioate
  modified_base 19: mod_base = OTHER; note = 2'-O-methyl adenosine
  modified_base 1: mod_base = OTHER; note = 2'-O-methyl guanosine 3'-phosphorothioate, conjugated at the 5' end to trishexylamino-N-Acetylgalactosamine-6-aminohexyl phosphodiester conjugate
gggacagtattctcagtga

SEQ ID NO: 46 - RNA, 19 nt, synthetic construct
  source 1..19: mol_type = other RNA
  modified_base 2: mod_base = OTHER; note = 2'-O-methoxyethyl guanosine 3'-phosphorothioate
  modified_base 3: mod_base = OTHER; note = 2'-O-methyl guanosine 3'-phosphodiester
  modified_base 4: mod_base = OTHER; note = 2'-O-methyl adenosine 3'-phosphodiester
  modified_base 5: mod_base = OTHER; note = 2'-O-methyl cytidine 3'-phosphodiester
  modified_base 6: mod_base = OTHER; note = 2'-O-methyl adenosine 3'-phosphodiester
  modified_base 7: mod_base = OTHER; note = 2'-O-methyl guanosine 3'-phosphodiester
  modified_base 8: mod_base = OTHER; note = 2'-O-methyl uridine 3'-phosphodiester
  modified_base 9: mod_base = OTHER; note = 2'-O-methyl adenosine 3'-phosphodiester
  modified_base 10: mod_base = OTHER; note = 2'-fluoro uridine 3'-phosphorothioate
  modified_base 11: mod_base = OTHER; note = 2'-fluoro uridine 3'-phosphodiester
  modified_base 12: mod_base = OTHER; note = 2'-O-methyl cytidine 3'-phosphodiester
  modified_base 13: mod_base = OTHER; note = 2'-O-methyl uridine 3'-phosphodiester
  modified_base 14: mod_base = OTHER; note = 2'-O-methyl cytidine 3'-phosphodiester
  modified_base 15: mod_base = OTHER; note = 2'-O-methyl adenosine 3'-phosphodiester
  modified_base 16: mod_base = OTHER; note = 2'-O-methyl guanosine 3'-phosphodiester
  modified_base 17: mod_base = OTHER; note = 2'-O-methyl uridine 3'-phosphorothioate
  modified_base 18: mod_base = OTHER; note = 2'-O-methoxyethyl guanosine 3'-phosphorothioate
  modified_base 19: mod_base = OTHER; note = 2'-O-methoxyethyl adenosine
  modified_base 1: mod_base = OTHER; note = 2'-O-methoxyethyl guanosine 3'-phosphorothioate, conjugated at the 5' end to trishexylamino-N-Acetylgalactosamine-6-aminohexyl phosphodiester conjugate
gggacagtattctcagtga

SEQ ID NO: 47 - RNA, 19 nt, synthetic construct
  source 1..19: mol_type = other RNA
  modified_base 2: mod_base = OTHER; note = 2'-O-methoxyethyl guanosine 3'-phosphorothioate
  modified_base 3: mod_base = OTHER; note = 2'-O-methyl guanosine 3'-phosphodiester
  modified_base 4: mod_base = OTHER; note = 2'-O-methyl adenosine 3'-phosphodiester
  modified_base 5: mod_base = OTHER; note = 2'-O-methyl cytidine 3'-phosphodiester
  modified_base 6: mod_base = OTHER; note = 2'-O-methyl adenosine 3'-phosphodiester
  modified_base 7: mod_base = OTHER; note = 2'-O-methyl guanosine 3'-phosphodiester
  modified_base 8: mod_base = OTHER; note = 2'-O-methyl uridine 3'-phosphodiester
  modified_base 9: mod_base = OTHER; note = 2'-fluoro adenosine 3'-phosphorothioate
  modified_base 10: mod_base = OTHER; note = 2'-fluoro uridine 3'-phosphorothioate
  modified_base 11: mod_base = OTHER; note = 2'-O-methyl uridine 3'-phosphodiester
  modified_base 12: mod_base = OTHER; note = 2'-O-methyl cytidine 3'-phosphodiester
  modified_base 13: mod_base = OTHER; note = 2'-O-methyl uridine 3'-phosphodiester
  modified_base 14: mod_base = OTHER; note = 2'-O-methyl cytidine 3'-phosphodiester
  modified_base 15: mod_base = OTHER; note = 2'-O-methyl adenosine 3'-phosphodiester
  modified_base 16: mod_base = OTHER; note = 2'-O-methyl guanosine 3'-phosphodiester
  modified_base 17: mod_base = OTHER; note = 2'-O-methyl uridine 3'-phosphorothioate
  modified_base 18: mod_base = OTHER; note = 2'-O-methoxyethyl guanosine 3'-phosphorothioate
  modified_base 19: mod_base = OTHER; note = 2'-O-methoxyethyl adenosine
  modified_base 1: mod_base = OTHER; note = 2'-O-methoxyethyl guanosine 3'-phosphorothioate, conjugated at the 5' end to trishexylamino-N-Acetylgalactosamine-6-aminohexyl phosphodiester conjugate
gggacagtattctcagtga

SEQ ID NO: 48 - RNA, 22 nt, synthetic construct
  source 1..22: mol_type = other RNA
  modified_base 2..3: mod_base = OTHER; note = 2'-O-methyl adenosine 3'-phosphorothioate
  modified_base 4..6: mod_base = OTHER; note = 2'-O-methyl guanosine 3'-phosphodiester
  modified_base 7: mod_base = OTHER; note = 2'-O-methyl adenosine 3'-phosphodiester
  modified_base 8: mod_base = OTHER; note = 2'-fluoro cytidine 3'-phosphodiester
  modified_base 9: mod_base = OTHER; note = 2'-O-methyl adenosine 3'-phosphodiester
  modified_base 10: mod_base = OTHER; note = 2'-fluoro guanosine 3'-phosphodiester
  modified_base 11: mod_base = OTHER; note = 2'-fluoro uridine 3'-phosphodiester
  modified_base 12: mod_base = OTHER; note = 2'-fluoro adenosine 3'-phosphodiester
  modified_base 13..14: mod_base = OTHER; note = 2'-O-methyl uridine 3'-phosphodiester
  modified_base 15: mod_base = OTHER; note = 2'-O-methyl cytidine 3'-phosphodiester
  modified_base 16: mod_base = OTHER; note = 2'-O-methyl uridine 3'-phosphodiester
  modified_base 17: mod_base = OTHER; note = 2'-O-methyl cytidine 3'-phosphodiester
  modified_base 18: mod_base = OTHER; note = 2'-O-methyl adenosine 3'-phosphodiester
  modified_base 19: mod_base = OTHER; note = 2'-O-methyl guanosine 3'-phosphodiester
  modified_base 20: mod_base = OTHER; note = 2'-O-methyl uridine 3'-phosphorothioate
  modified_base 21: mod_base = OTHER; note = 2'-O-methyl guanosine 3'-phosphorothioate
  modified_base 22: mod_base = OTHER; note = 2'-O-methyl adenosine
  modified_base 1: mod_base = OTHER; note = 2'-deoxythymidine 3'-phosphodiester, conjugated at the 5' end to trishexylamino-N-Acetylgalactosamine-6-aminohexyl phosphodiester conjugate
taagggacagtattctcagtga

SEQ ID NO: 49 - RNA, 21 nt, synthetic construct
  source 1..21: mol_type = other RNA
  modified_base 3..5: mod_base = OTHER; note = 2'-O-methyl guanosine 3'-phosphodiester
  modified_base 6: mod_base = OTHER; note = 2'-O-methyl adenosine 3'-phosphodiester
  modified_base 7: mod_base = OTHER; note = 2'-fluoro cytidine 3'-phosphodiester
  modified_base 8: mod_base = OTHER; note = 2'-O-methyl adenosine 3'-phosphodiester
  modified_base 9: mod_base = OTHER; note = 2'-fluoro guanosine 3'-phosphodiester
  modified_base 10: mod_base = OTHER; note = 2'-fluoro uridine 3'-phosphodiester
  modified_base 11: mod_base = OTHER; note = 2'-fluoro adenosine 3'-phosphodiester
  modified_base 12..13: mod_base = OTHER; note = 2'-O-methyl uridine 3'-phosphodiester
  modified_base 14: mod_base = OTHER; note = 2'-O-methyl cytidine 3'-phosphodiester
  modified_base 15: mod_base = OTHER; note = 2'-O-methyl uridine 3'-phosphodiester
  modified_base 16: mod_base = OTHER; note = 2'-O-methyl cytidine 3'-phosphodiester
  modified_base 17: mod_base = OTHER; note = 2'-O-methyl adenosine 3'-phosphodiester
  modified_base 18: mod_base = OTHER; note = 2'-O-methyl guanosine 3'-phosphodiester
  modified_base 19: mod_base = OTHER; note = 2'-O-methyl uridine 3'-phosphorothioate
  modified_base 20: mod_base = OTHER; note = 2'-O-methyl guanosine 3'-phosphorothioate
  modified_base 21: mod_base = OTHER; note = 2'-O-methyl adenosine phosphodiester, conjugated at the 3' end to trishexylamino-N-Acetylgalactosamine-hydroxyproline phosphodiester conjugate
  modified_base 1..2: mod_base = OTHER; note = 2'-O-methyl adenosine 3'-phosphorothioate
aagggacagtattctcagtga

SEQ ID NO: 50 - RNA, 23 nt, synthetic construct
  source 1..23: mol_type = other RNA
  modified_base 21: mod_base = i
  modified_base 1: mod_base = OTHER; note = inverted abasic 2'-beta-D-deoxyribose 3'-phosphorothioate
  modified_base 2: mod_base = OTHER; note = 2'-O-methyl adenosine 3'-phosphodiester
  modified_base 3: mod_base = OTHER; note = 2'-O-methyl cytidine 3'-phosphodiester
  modified_base 4..6: mod_base = OTHER; note = 2'-O-methyl guanosine 3'-phosphodiester
  modified_base 7: mod_base = OTHER; note = 2'-O-methyl adenosine 3'-phosphodiester
  modified_base 8: mod_base = OTHER; note = 2'-O-methyl cytidine 3'-phosphodiester
  modified_base 9: mod_base = OTHER; note = 2'-O-methyl adenosine 3'-phosphodiester
  modified_base 10: mod_base = OTHER; note = 2'-fluoro guanosine 3'-phosphodiester
  modified_base 11: mod_base = OTHER; note = 2'-fluoro uridine 3'-phosphodiester
  modified_base 12: mod_base = OTHER; note = 2'-fluoro adenosine 3'-phosphodiester
  modified_base 13..14: mod_base = OTHER; note = 2'-O-methyl uridine 3'-phosphodiester
  modified_base 15: mod_base = OTHER; note = 2'-O-methyl cytidine 3'-phosphodiester
  modified_base 16: mod_base = OTHER; note = 2'-O-methyl uridine 3'-phosphodiester
  modified_base 17: mod_base = OTHER; note = 2'-O-methyl cytidine 3'-phosphodiester
  modified_base 18: mod_base = OTHER; note = 2'-O-methyl adenosine 3'-phosphodiester
  modified_base 19: mod_base = OTHER; note = 2'-O-methyl guanosine 3'-phosphodiester
  modified_base 20: mod_base = OTHER; note = 2'-O-methyl uridine 3'-phosphodiester
  modified_base 21: mod_base = OTHER; note = 2'-O-methyl inosine 3'-phosphodiester
  modified_base 22: mod_base = OTHER; note = 2'-O-methyl adenosine phosphorothioate
  modified_base 23: mod_base = OTHER; note = inverted abasic 2'-beta-D-deoxyribose
nacgggacagtattctcagtnan

SEQ ID NO: 51 - RNA, 23 nt, synthetic construct
  source 1..23: mol_type = other RNA
  modified_base 1: mod_base = OTHER; note = 2'-O-methoxyethyl thymidine 3'-phosphorothioate
  modified_base 2: mod_base = OTHER; note = 2'-fluoro cytidine 3'-phosphorothioate
  modified_base 3: mod_base = OTHER; note = 2'-O-methyl adenosine 3'-phosphodiester
  modified_base 4: mod_base = OTHER; note = 2'-O-methyl cytidine 3'-phosphodiester
  modified_base 5: mod_base = OTHER; note = 2'-O-methyl uridine 3'-phosphodiester
  modified_base 6: mod_base = OTHER; note = 2'-fluoro guanosine 3'-phosphodiester
  modified_base 7: mod_base = OTHER; note = 2'-O-methyl adenosine 3'-phosphodiester
  modified_base 8: mod_base = OTHER; note = 2'-O-methyl guanosine 3'-phosphodiester
  modified_base 9..10: mod_base = OTHER; note = 2'-O-methyl adenosine 3'-phosphodiester
  modified_base 11: mod_base = OTHER; note = 2'-O-methyl uridine 3'-phosphodiester
  modified_base 12: mod_base = OTHER; note = 2'-O-methyl adenosine 3'-phosphodiester
  modified_base 13: mod_base = OTHER; note = 2'-O-methyl cytidine 3'-phosphodiester
  modified_base 14: mod_base = OTHER; note = 2'-fluoro uridine 3'-phosphodiester
  modified_base 15: mod_base = OTHER; note = 2'-O-methyl guanosine 3'-phosphodiester
  modified_base 16: mod_base = OTHER; note = 2'-fluoro uridine 3'-phosphodiester
  modified_base 17..19: mod_base = OTHER; note = 2'-O-methyl cytidine 3'-phosphodiester
  modified_base 20: mod_base = OTHER; note = 2'-O-methyl uridine 3'-phosphodiester
  modified_base 21..22: mod_base = OTHER; note = 2'-O-methyl uridine 3'-phosphorothioate
  modified_base 23: mod_base = OTHER; note = 2'-O-methyl uridine
tcactgagaatactgtccctttt

SEQ ID NO: 52 - RNA, 23 nt, synthetic construct
  source 1..23: mol_type = other RNA
  modified_base 1: mod_base = OTHER; note = 2'-O-methoxyethyl thymidine 3'-phosphorothioate
  modified_base 2: mod_base = OTHER; note = 2'-fluoro cytidine 3'-phosphorothioate
  modified_base 3: mod_base = OTHER; note = 2'-O-methyl adenosine 3'-phosphodiester
  modified_base 4: mod_base = OTHER; note = 2'-O-methyl cytidine 3'-phosphodiester
  modified_base 5: mod_base = OTHER; note = 2'-O-methyl uridine 3'-phosphodiester
  modified_base 6: mod_base = OTHER; note = 2'-deoxyguanosine 3'-phosphodiester
  modified_base 7: mod_base = OTHER; note = 2'-O-methyl adenosine 3'-phosphodiester
  modified_base 8: mod_base = OTHER; note = 2'-O-methyl guanosine 3'-phosphodiester
  modified_base 9..10: mod_base = OTHER; note = 2'-O-methyl adenosine 3'-phosphodiester
  modified_base 11: mod_base = OTHER; note = 2'-O-methyl uridine 3'-phosphodiester
  modified_base 12: mod_base = OTHER; note = 2'-O-methyl adenosine 3'-phosphodiester
  modified_base 13: mod_base = OTHER; note = 2'-O-methyl cytidine 3'-phosphodiester
  modified_base 14: mod_base = OTHER; note = 2'-fluoro uridine 3'-phosphodiester
  modified_base 15: mod_base = OTHER; note = 2'-O-methyl guanosine 3'-phosphodiester
  modified_base 16: mod_base = OTHER; note = 2'-fluoro uridine 3'-phosphodiester
  modified_base 17..19: mod_base = OTHER; note = 2'-O-methyl cytidine 3'-phosphodiester
  modified_base 20: mod_base = OTHER; note = 2'-O-methyl uridine 3'-phosphodiester
  modified_base 21..22: mod_base = OTHER; note = 2'-O-methyl uridine 3'-phosphorothioate
  modified_base 23: mod_base = OTHER; note = 2'-O-methyl uridine
tcactgagaatactgtccctttt

SEQ ID NO: 53 - RNA, 23 nt, synthetic construct
  source 1..23: mol_type = other RNA
  modified_base 1: mod_base = OTHER; note = 2'-O-methoxyethyl thymidine 3'-phosphorothioate
  modified_base 2: mod_base = OTHER; note = 2'-fluoro cytidine 3'-phosphorothioate
  modified_base 3: mod_base = OTHER; note = 2'-O-methyl adenosine 3'-phosphodiester
  modified_base 4: mod_base = OTHER; note = 2'-O-methyl cytidine 3'-phosphodiester
  modified_base 5: mod_base = OTHER; note = 2'-O-methyl uridine 3'-phosphodiester
  modified_base 6: mod_base = OTHER; note = 2'-deoxyguanosine 3'-phosphorothioate
  modified_base 7: mod_base = OTHER; note = 2'-O-methyl adenosine 3'-phosphodiester
  modified_base 8: mod_base = OTHER; note = 2'-O-methyl guanosine 3'-phosphodiester
  modified_base 9..10: mod_base = OTHER; note = 2'-O-methyl adenosine 3'-phosphodiester
  modified_base 11: mod_base = OTHER; note = 2'-O-methyl uridine 3'-phosphodiester
  modified_base 12: mod_base = OTHER; note = 2'-O-methyl adenosine 3'-phosphodiester
  modified_base 13: mod_base = OTHER; note = 2'-O-methyl cytidine 3'-phosphodiester
  modified_base 14: mod_base = OTHER; note = 2'-fluoro uridine 3'-phosphodiester
  modified_base 15: mod_base = OTHER; note = 2'-O-methyl guanosine 3'-phosphodiester
  modified_base 16: mod_base = OTHER; note = 2'-fluoro uridine 3'-phosphodiester
  modified_base 17..19: mod_base = OTHER; note = 2'-O-methyl cytidine 3'-phosphodiester
  modified_base 20: mod_base = OTHER; note = 2'-O-methyl uridine 3'-phosphodiester
  modified_base 21..22: mod_base = OTHER; note = 2'-O-methyl uridine 3'-phosphorothioate
  modified_base 23: mod_base = OTHER; note = 2'-O-methyl uridine
tcactgagaatactgtccctttt

SEQ ID NO: 54 - RNA, 23 nt, synthetic construct
  source 1..23: mol_type = other RNA
  modified_base 1: mod_base = OTHER; note = 2'-O-methoxyethyl thymidine 3'-phosphorothioate
  modified_base 2: mod_base = OTHER; note = 2'-fluoro cytidine 3'-phosphorothioate
  modified_base 3: mod_base = OTHER; note = 2'-O-methyl adenosine 3'-phosphodiester
  modified_base 4: mod_base = OTHER; note = 2'-O-methyl cytidine 3'-phosphodiester
  modified_base 5: mod_base = OTHER; note = 2'-O-methyl uridine 3'-phosphodiester
  modified_base 6: mod_base = OTHER; note = 2'-deoxyguanosine 3'-phosphodiester
  modified_base 7: mod_base = OTHER; note = 2'-O-methyl adenosine 3'-phosphodiester
  modified_base 8: mod_base = OTHER; note = 2'-O-methyl guanosine 3'-phosphodiester
  modified_base 9..10: mod_base = OTHER; note = 2'-O-methyl adenosine 3'-phosphodiester
  modified_base 11: mod_base = OTHER; note = 2'-O-methyl uridine 3'-phosphodiester
  modified_base 12: mod_base = OTHER; note = 2'-O-methyl adenosine 3'-phosphodiester
  modified_base 13: mod_base = OTHER; note = 2'-O-methyl cytidine 3'-phosphodiester
  modified_base 14: mod_base = OTHER; note = 2'-deoxyuridine 3'-phosphodiester
  modified_base 15: mod_base = OTHER; note = 2'-O-methyl guanosine 3'-phosphodiester
  modified_base 16: mod_base = OTHER; note = 2'-deoxyuridine 3'-phosphodiester
  modified_base 17..19: mod_base = OTHER; note = 2'-O-methyl cytidine 3'-phosphodiester
  modified_base 20: mod_base = OTHER; note = 2'-O-methyl uridine 3'-phosphodiester
  modified_base 21..22: mod_base = OTHER; note = 2'-O-methyl uridine 3'-phosphorothioate
  modified_base 23: mod_base = OTHER; note = 2'-O-methyl uridine
tcactgagaatactgtccctttt

SEQ ID NO: 55 - RNA, 23 nt, synthetic construct
  source 1..23: mol_type = other RNA
  modified_base 1: mod_base = OTHER; note = 2'-O-methoxyethyl thymidine 3'-phosphorothioate
  modified_base 2: mod_base = OTHER; note = 2'-fluoro cytidine 3'-phosphorothioate
  modified_base 3: mod_base = OTHER; note = 2'-O-methyl adenosine 3'-phosphodiester
  modified_base 4: mod_base = OTHER; note = 2'-O-methyl cytidine 3'-phosphodiester
  modified_base 5: mod_base = OTHER; note = 2'-O-methyl uridine 3'-phosphodiester
  modified_base 6: mod_base = OTHER; note = 2'-deoxyguanosine 3'-phosphorothioate
  modified_base 7: mod_base = OTHER; note = 2'-O-methyl adenosine 3'-phosphodiester
  modified_base 8: mod_base = OTHER; note = 2'-O-methyl guanosine 3'-phosphodiester
  modified_base 9..10: mod_base = OTHER; note = 2'-O-methyl adenosine 3'-phosphodiester
  modified_base 11: mod_base = OTHER; note = 2'-O-methyl uridine 3'-phosphodiester
  modified_base 12: mod_base = OTHER; note = 2'-O-methyl adenosine 3'-phosphodiester
  modified_base 13: mod_base = OTHER; note = 2'-O-methyl cytidine 3'-phosphodiester
  modified_base 14: mod_base = OTHER; note = 2'-deoxyuridine 3'-phosphorothioate
  modified_base 15: mod_base = OTHER; note = 2'-O-methyl guanosine 3'-phosphodiester
  modified_base 16: mod_base = OTHER; note = 2'-deoxyuridine 3'-phosphorothioate
  modified_base 17..19: mod_base = OTHER; note = 2'-O-methyl cytidine 3'-phosphodiester
  modified_base 20: mod_base = OTHER; note = 2'-O-methyl uridine 3'-phosphodiester
  modified_base 21..22: mod_base = OTHER; note = 2'-O-methyl uridine 3'-phosphorothioate
  modified_base 23: mod_base = OTHER; note = 2'-O-methyl uridine
tcactgagaatactgtccctttt

SEQ ID NO: 56 - RNA, 23 nt, synthetic construct
  source 1..23: mol_type = other RNA
  modified_base 1: mod_base = OTHER; note = 2'-O-methoxyethyl thymidine 3'-phosphorothioate
  modified_base 2: mod_base = OTHER; note = 2'-fluoro cytidine 3'-phosphorothioate
  modified_base 3: mod_base = OTHER; note = 2'-O-methyl adenosine 3'-phosphodiester
  modified_base 4: mod_base = OTHER; note = 2'-O-methyl cytidine 3'-phosphodiester
  modified_base 5: mod_base = OTHER; note = 2'-O-methyl uridine 3'-phosphodiester
  modified_base 6: mod_base = OTHER; note = 2'-O-methyl guanosine 3'-phosphodiester
  modified_base 7: mod_base = OTHER; note = 2'-O-methyl adenosine 3'-phosphodiester
  modified_base 8: mod_base = OTHER; note = 2'-O-methyl guanosine 3'-phosphodiester
  modified_base 9..10: mod_base = OTHER; note = 2'-O-methyl adenosine 3'-phosphodiester
  modified_base 11: mod_base = OTHER; note = 2'-O-methyl uridine 3'-phosphodiester
  modified_base 12: mod_base = OTHER; note = 2'-O-methyl adenosine 3'-phosphodiester
  modified_base 13: mod_base = OTHER; note = 2'-O-methyl cytidine 3'-phosphodiester
  modified_base 14: mod_base = OTHER; note = 2'-fluoro uridine 3'-phosphodiester
  modified_base 15: mod_base = OTHER; note = 2'-O-methyl guanosine 3'-phosphodiester
  modified_base 16: mod_base = OTHER; note = 2'-O-methyl uridine 3'-phosphodiester
  modified_base 17..19: mod_base = OTHER; note = 2'-O-methyl cytidine 3'-phosphodiester
  modified_base 20: mod_base = OTHER; note = 2'-O-methyl uridine 3'-phosphodiester
  modified_base 21..22: mod_base = OTHER; note = 2'-O-methyl uridine 3'-phosphorothioate
  modified_base 23: mod_base = OTHER; note = 2'-O-methyl uridine
tcactgagaatactgtccctttt

SEQ ID NO: 57 - RNA, 23 nt, synthetic construct
  source 1..23: mol_type = other RNA
  modified_base 1: mod_base = OTHER; note = 2'-O-methoxyethyl thymidine 3'-phosphorothioate
  modified_base 2: mod_base = OTHER; note = 2'-fluoro cytidine 3'-phosphorothioate
  modified_base 3: mod_base = OTHER; note = 2'-O-methyl adenosine 3'-phosphodiester
  modified_base 4: mod_base = OTHER; note = 2'-O-methyl cytidine 3'-phosphodiester
  modified_base 5: mod_base = OTHER; note = 2'-O-methyl uridine 3'-phosphodiester
  modified_base 6: mod_base = OTHER; note = 2'-fluoro guanosine 3'-phosphodiester
  modified_base 7: mod_base = OTHER; note = 2'-O-methyl adenosine 3'-phosphodiester
  modified_base 8: mod_base = OTHER; note = 2'-O-methyl guanosine 3'-phosphodiester
  modified_base 9: mod_base = OTHER; note = 2'-O-methoxyethyl adenosine 3'-phosphodiester
  modified_base 10: mod_base = OTHER; note = 2'-O-methyl adenosine 3'-phosphodiester
  modified_base 11: mod_base = OTHER; note = 2'-O-methyl uridine 3'-phosphodiester
  modified_base 12: mod_base = OTHER; note = 2'-O-methyl adenosine 3'-phosphodiester
  modified_base 13: mod_base = OTHER; note = 2'-O-methyl cytidine 3'-phosphodiester
  modified_base 14: mod_base = OTHER; note = 2'-fluoro uridine 3'-phosphodiester
  modified_base 15: mod_base = OTHER; note = 2'-O-methyl guanosine 3'-phosphodiester
  modified_base 16: mod_base = OTHER; note = 2'-fluoro uridine 3'-phosphodiester
  modified_base 17..19: mod_base = OTHER; note = 2'-O-methyl cytidine 3'-phosphodiester
  modified_base 20: mod_base = OTHER; note = 2'-O-methyl uridine 3'-phosphodiester
  modified_base 21..22: mod_base = OTHER; note = 2'-O-methyl uridine 3'-phosphorothioate
  modified_base 23: mod_base = OTHER; note = 2'-O-methyl uridine
tcactgagaatactgtccctttt

SEQ ID NO: 58 - RNA, 23 nt, synthetic construct
  source 1..23: mol_type = other RNA
  modified_base 1: mod_base = OTHER; note = 2'-O-methoxyethyl thymidine 3'-phosphorothioate
  modified_base 2: mod_base = OTHER; note = 2'-fluoro cytidine 3'-phosphorothioate
  modified_base 3: mod_base = OTHER; note = 2'-O-methyl adenosine 3'-phosphodiester
  modified_base 4: mod_base = OTHER; note = 2'-O-methyl cytidine 3'-phosphodiester
  modified_base 5: mod_base = OTHER; note = 2'-O-methyl uridine 3'-phosphodiester
  modified_base 6: mod_base = OTHER; note = 2'-fluoro guanosine 3'-phosphodiester
  modified_base 7: mod_base = OTHER; note = 2'-O-methyl adenosine 3'-phosphodiester
  modified_base 8: mod_base = OTHER; note = 2'-O-methyl guanosine 3'-phosphodiester
  modified_base 9..10: mod_base = OTHER; note = 2'-O-methoxyethyl adenosine 3'-phosphodiester
  modified_base 11: mod_base = OTHER; note = 2'-O-methyl uridine 3'-phosphodiester
  modified_base 12: mod_base = OTHER; note = 2'-O-methyl adenosine 3'-phosphodiester
  modified_base 13: mod_base = OTHER; note = 2'-O-methyl cytidine 3'-phosphodiester
  modified_base 14: mod_base = OTHER; note = 2'-fluoro uridine 3'-phosphodiester
  modified_base 15: mod_base = OTHER; note = 2'-O-methyl guanosine 3'-phosphodiester
  modified_base 16: mod_base = OTHER; note = 2'-fluoro uridine 3'-phosphodiester
  modified_base 17..19: mod_base = OTHER; note = 2'-O-methyl cytidine 3'-phosphodiester
  modified_base 20: mod_base = OTHER; note = 2'-O-methyl uridine 3'-phosphodiester
  modified_base 21..22: mod_base = OTHER; note = 2'-O-methyl uridine 3'-phosphorothioate
  modified_base 23: mod_base = OTHER; note = 2'-O-methyl uridine
tcactgagaatactgtccctttt

SEQ ID NO: 59 - RNA, 23 nt, synthetic construct
  source 1..23: mol_type = other RNA
  modified_base 1: mod_base = OTHER; note = 2'-O-methoxyethyl thymidine 3'-phosphorothioate
  modified_base 2: mod_base = OTHER; note = 2'-fluoro cytidine 3'-phosphorothioate
  modified_base 3: mod_base = OTHER; note = 2'-O-methyl adenosine 3'-phosphodiester
  modified_base 4: mod_base = OTHER; note = 2'-O-methyl cytidine 3'-phosphodiester
  modified_base 5: mod_base = OTHER; note = 2'-O-methyl uridine 3'-phosphodiester
  modified_base 6: mod_base = OTHER; note = 2'-fluoro guanosine 3'-phosphodiester
  modified_base 7: mod_base = OTHER; note = 2'-O-methyl adenosine 3'-phosphodiester
  modified_base 8: mod_base = OTHER; note = 2'-O-methyl guanosine 3'-phosphodiester
  modified_base 9..10: mod_base = OTHER; note = 2'-O-methyl adenosine 3'-phosphodiester
  modified_base 11: mod_base = OTHER; note = 2'-O-methyl uridine 3'-phosphodiester
  modified_base 12: mod_base = OTHER; note = 2'-O-methyl adenosine 3'-phosphodiester
  modified_base 13: mod_base = OTHER; note = 2'-O-methyl cytidine 3'-phosphodiester
  modified_base 14: mod_base = OTHER; note = 2'-fluoro uridine 3'-phosphodiester
  modified_base 15: mod_base = OTHER; note = 2'-O-methyl guanosine 3'-phosphodiester
  modified_base 16: mod_base = OTHER; note = 2'-fluoro uridine 3'-phosphodiester
  modified_base 17..19: mod_base = OTHER; note = 2'-O-methyl cytidine 3'-phosphodiester
  modified_base 20: mod_base = OTHER; note = 2'-O-methyl uridine 3'-phosphodiester
  modified_base 21..22: mod_base = OTHER; note = 2'-O-methyl uridine 3'-phosphorothioate
  modified_base 23: mod_base = OTHER; note = 2'-O-methyl uridine
tcactgagaatactgtccctttt

SEQ ID NO: 60 - RNA, 23 nt, synthetic construct
  source 1..23: mol_type = other RNA
  modified_base 1: mod_base = OTHER; note = 2'-O-methoxyethyl thymidine 3'-phosphorothioate
  modified_base 2: mod_base = OTHER; note = 2'-fluoro cytidine 3'-phosphorothioate
  modified_base 3: mod_base = OTHER; note = 2'-O-methyl adenosine 3'-phosphodiester
  modified_base 4: mod_base = OTHER; note = 2'-O-methyl cytidine 3'-phosphodiester
  modified_base 5: mod_base = OTHER; note = 2'-O-methyl uridine 3'-phosphodiester
  modified_base 6: mod_base = OTHER; note = 2'-deoxyguanosine 3'-phosphorothioate
  modified_base 7: mod_base = OTHER; note = 2'-O-methyl adenosine 3'-phosphodiester
  modified_base 8: mod_base = OTHER; note = 2'-O-methyl guanosine 3'-phosphodiester
  modified_base 9..10: mod_base = OTHER; note = 2'-O-methyl adenosine 3'-phosphodiester
  modified_base 11: mod_base = OTHER; note = 2'-O-methyl uridine 3'-phosphodiester
  modified_base 12: mod_base = OTHER; note = 2'-O-methyl adenosine 3'-phosphodiester
  modified_base 13: mod_base = OTHER; note = 2'-O-methyl cytidine 3'-phosphodiester
  modified_base 14: mod_base = OTHER; note = 2'-fluoro uridine 3'-phosphodiester
  modified_base 15: mod_base = OTHER; note = 2'-O-methyl guanosine 3'-phosphodiester
  modified_base 16: mod_base = OTHER; note = 2'-fluoro uridine 3'-phosphodiester
  modified_base 17..19: mod_base = OTHER; note = 2'-O-methyl cytidine 3'-phosphodiester
  modified_base 20: mod_base = OTHER; note = 2'-O-methyl uridine 3'-phosphodiester
  modified_base 21: mod_base = OTHER; note = 2'-O-methyl uridine 3'-phosphorothioate
  modified_base 22: mod_base = OTHER; note = 2'-O-methoxyethyl adenosine 3'-phosphorothioate
  modified_base 23: mod_base = OTHER; note = 2'-O-methoxyethyl adenosine
tcactgagaatactgtcccttaa

SEQ ID NO: 61 - RNA, 23 nt, synthetic construct
  source 1..23: mol_type = other RNA
  modified_base 1: mod_base = OTHER; note = 2'-O-methoxyethyl thymidine 3'-phosphorothioate
  modified_base 2: mod_base = OTHER; note = 2'-fluoro cytidine 3'-phosphorothioate
  modified_base 3: mod_base = OTHER; note = 2'-O-methyl adenosine 3'-phosphodiester
  modified_base 4: mod_base = OTHER; note = 2'-O-methyl cytidine 3'-phosphodiester
  modified_base 5: mod_base = OTHER; note = 2'-O-methyl uridine 3'-phosphodiester
  modified_base 6: mod_base = OTHER; note = 2'-deoxyguanosine 3'-phosphorothioate
  modified_base 7: mod_base = OTHER; note = 2'-O-methyl adenosine 3'-phosphodiester
  modified_base 8: mod_base = OTHER; note = 2'-O-methyl guanosine 3'-phosphodiester
  modified_base 9..10: mod_base = OTHER; note = 2'-O-methyl adenosine 3'-phosphodiester
  modified_base 11: mod_base = OTHER; note = 2'-O-methyl uridine 3'-phosphodiester
  modified_base 12: mod_base = OTHER; note = 2'-O-methyl adenosine 3'-phosphodiester
  modified_base 13: mod_base = OTHER; note = 2'-O-methyl cytidine 3'-phosphodiester
  modified_base 14: mod_base = OTHER; note = 2'-deoxyuridine 3'-phosphorothioate
  modified_base 15: mod_base = OTHER; note = 2'-O-methyl guanosine 3'-phosphodiester
  modified_base 16: mod_base = OTHER; note = 2'-deoxyuridine 3'-phosphorothioate
  modified_base 17..19: mod_base = OTHER; note = 2'-O-methyl cytidine 3'-phosphodiester
  modified_base 20: mod_base = OTHER; note = 2'-O-methyl uridine 3'-phosphodiester
  modified_base 21: mod_base = OTHER; note = 2'-O-methyl uridine 3'-phosphorothioate
  modified_base 22: mod_base = OTHER; note = 2'-O-methoxyethyl adenosine 3'-phosphorothioate
  modified_base 23: mod_base = OTHER; note = 2'-O-methoxyethyl adenosine
tcactgagaatactgtcccttaa

SEQ ID NO: 62 - RNA, 23 nt, synthetic construct
  source 1..23: mol_type = other RNA
  modified_base 1: mod_base = OTHER; note = 2'-O-methoxyethyl thymidine 3'-phosphorothioate
  modified_base 2: mod_base = OTHER; note = 2'-fluoro cytidine 3'-phosphorothioate
  modified_base 3: mod_base = OTHER; note = 2'-O-methyl adenosine 3'-phosphodiester
  modified_base 4: mod_base = OTHER; note = 2'-O-methyl cytidine 3'-phosphodiester
  modified_base 5: mod_base = OTHER; note = 2'-O-methyl uridine 3'-phosphodiester
  modified_base 6: mod_base = OTHER; note = 2'-deoxyguanosine 3'-phosphodiester
  modified_base 7: mod_base = OTHER; note = 2'-O-methyl adenosine 3'-phosphodiester
  modified_base 8: mod_base = OTHER; note = 2'-O-methyl guanosine 3'-phosphodiester
  modified_base 9..10: mod_base = OTHER; note = 2'-O-methyl adenosine 3'-phosphodiester
  modified_base 11: mod_base = OTHER; note = 2'-O-methyl uridine 3'-phosphodiester
  modified_base 12: mod_base = OTHER; note = 2'-O-methyl adenosine 3'-phosphodiester
  modified_base 13: mod_base = OTHER; note = 2'-O-methyl cytidine 3'-phosphodiester
  modified_base 14: mod_base = OTHER; note = 2'-fluoro uridine 3'-phosphodiester
  modified_base 15: mod_base = OTHER; note = 2'-O-methyl guanosine 3'-phosphodiester
  modified_base 16: mod_base = OTHER; note = 2'-fluoro uridine 3'-phosphodiester
  modified_base 17..19: mod_base = OTHER; note = 2'-O-methyl cytidine 3'-phosphodiester
  modified_base 20: mod_base = OTHER; note = 2'-O-methyl uridine 3'-phosphodiester
  modified_base 21: mod_base = OTHER; note = 2'-O-methyl uridine 3'-phosphorothioate
  modified_base 22: mod_base = OTHER; note = 2'-O-methoxyethyl adenosine 3'-phosphorothioate
  modified_base 23: mod_base = OTHER; note = 2'-O-methoxyethyl adenosine
tcactgagaatactgtcccttaa

SEQ ID NO: 63 - RNA, 23 nt, synthetic construct
  source 1..23: mol_type = other RNA
  modified_base 1: mod_base = OTHER; note = 2'-O-methoxyethyl thymidine 3'-phosphorothioate
  modified_base 2: mod_base = OTHER; note = 2'-fluoro cytidine 3'-phosphorothioate
  modified_base 3: mod_base = OTHER; note = 2'-O-methyl adenosine 3'-phosphodiester
  modified_base 4: mod_base = OTHER; note = 2'-O-methyl cytidine 3'-phosphodiester
  modified_base 5: mod_base = OTHER; note = 2'-O-methyl uridine 3'-phosphodiester
  modified_base 6: mod_base = OTHER; note = 2'-deoxyguanosine 3'-phosphodiester
  modified_base 7: mod_base = OTHER; note = 2'-O-methyl adenosine 3'-phosphodiester
  modified_base 8: mod_base = OTHER; note = 2'-O-methyl guanosine 3'-phosphodiester
  modified_base 9..10: mod_base = OTHER; note = 2'-O-methyl adenosine 3'-phosphodiester
  modified_base 11: mod_base = OTHER; note = 2'-O-methyl uridine 3'-phosphodiester
  modified_base 12: mod_base = OTHER; note = 2'-O-methyl adenosine 3'-phosphodiester
  modified_base 13: mod_base = OTHER; note = 2'-O-methyl cytidine 3'-phosphodiester
  modified_base 14: mod_base = OTHER; note = 2'-deoxyuridine 3'-phosphodiester
  modified_base 15: mod_base = OTHER; note = 2'-O-methyl guanosine 3'-phosphodiester
  modified_base 16: mod_base = OTHER; note = 2'-deoxyuridine 3'-phosphodiester
  modified_base 17..19: mod_base = OTHER; note = 2'-O-methyl cytidine 3'-phosphodiester
  modified_base 20: mod_base = OTHER; note = 2'-O-methyl uridine 3'-phosphodiester
  modified_base 21: mod_base = OTHER; note = 2'-O-methyl uridine 3'-phosphorothioate
  modified_base 22: mod_base = OTHER; note = 2'-O-methoxyethyl adenosine 3'-phosphorothioate
  modified_base 23: mod_base = OTHER; note = 2'-O-methoxyethyl adenosine
tcactgagaatactgtcccttaa

SEQ ID NO: 64 - RNA, 23 nt, synthetic construct
  source 1..23: mol_type = other RNA
  modified_base 1: mod_base = OTHER; note = 2'-O-methoxyethyl thymidine 3'-phosphorothioate
  modified_base 2: mod_base = OTHER; note = 2'-fluoro cytidine 3'-phosphorothioate
  modified_base 3: mod_base = OTHER; note = 2'-O-methyl adenosine 3'-phosphodiester
  modified_base 4: mod_base = OTHER; note = 2'-O-methyl cytidine 3'-phosphodiester
  modified_base 5: mod_base = OTHER; note = 2'-O-methyl uridine 3'-phosphodiester
  modified_base 6: mod_base = OTHER; note = 2'-deoxyguanosine 3'-phosphorothioate
  modified_base 7: mod_base = OTHER; note = 2'-O-methyl adenosine 3'-phosphodiester
  modified_base 8: mod_base = OTHER; note = 2'-O-methyl guanosine 3'-phosphodiester
  modified_base 9: mod_base = OTHER; note = 2'-O-methoxyethyl adenosine 3'-phosphodiester
  modified_base 10: mod_base = OTHER; note = 2'-O-methyl adenosine 3'-phosphodiester
  modified_base 11: mod_base = OTHER; note = 2'-O-methyl uridine 3'-phosphodiester
  modified_base 12: mod_base = OTHER; note = 2'-O-methyl adenosine 3'-phosphodiester
  modified_base 13: mod_base = OTHER; note = 2'-O-methyl cytidine 3'-phosphodiester
  modified_base 14: mod_base = OTHER; note = 2'-fluoro uridine 3'-phosphodiester
  modified_base 15: mod_base = OTHER; note = 2'-O-methyl guanosine 3'-phosphodiester
  modified_base 16: mod_base = OTHER; note = 2'-fluoro uridine 3'-phosphodiester
  modified_base 17..19: mod_base = OTHER; note = 2'-O-methyl cytidine 3'-phosphodiester
  modified_base 20: mod_base = OTHER; note = 2'-O-methyl uridine 3'-phosphodiester
  modified_base 21: mod_base = OTHER; note = 2'-O-methyl uridine 3'-phosphorothioate
  modified_base 22: mod_base = OTHER; note = 2'-O-methoxyethyl adenosine 3'-phosphorothioate
  modified_base 23: mod_base = OTHER; note = 2'-O-methoxyethyl adenosine
tcactgagaatactgtcccttaa

SEQ ID NO: 65 - RNA, 23 nt, synthetic construct
  source 1..23: mol_type = other RNA
  modified_base 1: mod_base = OTHER; note = 2'-O-methoxyethyl thymidine 3'-phosphorothioate
  modified_base 2: mod_base = OTHER; note = 2'-fluoro cytidine 3'-phosphorothioate
  modified_base 3: mod_base = OTHER; note = 2'-O-methyl adenosine 3'-phosphodiester
  modified_base 4: mod_base = OTHER; note = 2'-O-methyl cytidine 3'-phosphodiester
  modified_base 5: mod_base = OTHER; note = 2'-O-methyl uridine 3'-phosphodiester
  modified_base 6: mod_base = OTHER; note = 2'-deoxyguanosine 3'-phosphodiester
  modified_base 7: mod_base = OTHER; note = 2'-O-methyl adenosine 3'-phosphodiester
  modified_base 8: mod_base = OTHER; note = 2'-O-methyl guanosine 3'-phosphodiester
  modified_base 9: mod_base = OTHER; note = 2'-O-methoxyethyl adenosine 3'-phosphodiester
  modified_base 10: mod_base = OTHER; note = 2'-O-methyl adenosine 3'-phosphodiester
  modified_base 11: mod_base = OTHER; note = 2'-O-methyl uridine 3'-phosphodiester
  modified_base 12: mod_base = OTHER; note = 2'-O-methyl adenosine 3'-phosphodiester
  modified_base 13: mod_base = OTHER; note = 2'-O-methyl cytidine 3'-phosphodiester
  modified_base 14: mod_base = OTHER; note = 2'-deoxyuridine 3'-phosphodiester
  modified_base 15: mod_base = OTHER; note = 2'-O-methyl guanosine 3'-phosphodiester
  modified_base 16: mod_base = OTHER; note = 2'-deoxyuridine 3'-phosphodiester
  modified_base 17..19: mod_base = OTHER; note = 2'-O-methyl cytidine 3'-phosphodiester
  modified_base 20: mod_base = OTHER; note = 2'-O-methyl uridine 3'-phosphodiester
  modified_base 21: mod_base = OTHER; note = 2'-O-methyl uridine 3'-phosphorothioate
  modified_base 22: mod_base = OTHER; note = 2'-O-methoxyethyl adenosine 3'-phosphorothioate
  modified_base 23: mod_base = OTHER; note = 2'-O-methoxyethyl adenosine
tcactgagaatactgtcccttaa

SEQ ID NO: 66 - RNA, 23 nt, synthetic construct
  source 1..23: mol_type = other RNA
  modified_base 1: mod_base = OTHER; note = 2'-O-methoxyethyl thymidine 3'-phosphorothioate
  modified_base 2: mod_base = OTHER; note = 2'-fluoro cytidine 3'-phosphorothioate
  modified_base 3: mod_base = OTHER; note = 2'-O-methyl adenosine 3'-phosphodiester
  modified_base 4: mod_base = OTHER; note = 2'-O-methyl cytidine 3'-phosphodiester
  modified_base 5: mod_base = OTHER; note = 2'-O-methyl uridine 3'-phosphodiester
  modified_base 6: mod_base = OTHER; note = 2'-deoxyguanosine 3'-phosphodiester
  modified_base 7: mod_base = OTHER; note = 2'-O-methyl adenosine 3'-phosphodiester
  modified_base 8: mod_base = OTHER; note = 2'-O-methyl guanosine 3'-phosphodiester
  modified_base 9..10: mod_base = OTHER; note = 2'-O-methoxyethyl adenosine 3'-phosphodiester
  modified_base 11: mod_base = OTHER; note = 2'-O-methyl uridine 3'-phosphodiester
  modified_base 12: mod_base = OTHER; note = 2'-O-methyl adenosine 3'-phosphodiester
  modified_base 13: mod_base = OTHER; note = 2'-O-methyl cytidine 3'-phosphodiester
  modified_base 14: mod_base = OTHER; note = 2'-deoxyuridine 3'-phosphodiester
  modified_base 15: mod_base = OTHER; note = 2'-O-methyl guanosine 3'-phosphodiester
  modified_base 16: mod_base = OTHER; note = 2'-deoxyuridine 3'-phosphodiester
  modified_base 17..19: mod_base = OTHER; note = 2'-O-methyl cytidine 3'-phosphodiester
  modified_base 20: mod_base = OTHER; note = 2'-O-methyl uridine 3'-phosphodiester
  modified_base 21: mod_base = OTHER; note = 2'-O-methyl uridine 3'-phosphorothioate
  modified_base 22: mod_base = OTHER; note = 2'-O-methoxyethyl adenosine 3'-phosphorothioate
  modified_base 23: mod_base = OTHER; note = 2'-O-methoxyethyl adenosine
tcactgagaatactgtcccttaa

SEQ ID NO: 67 - RNA, 23 nt, synthetic construct
  source 1..23: mol_type = other RNA
  modified_base 1: mod_base = OTHER; note = 2'-O-methoxyethyl thymidine 3'-phosphorothioate
  modified_base 2: mod_base = OTHER; note = 2'-fluoro cytidine 3'-phosphorothioate
  modified_base 3: mod_base = OTHER; note = 2'-O-methyl adenosine 3'-phosphodiester
  modified_base 4: mod_base = OTHER; note = 2'-O-methyl cytidine 3'-phosphodiester
  modified_base 5: mod_base = OTHER; note = 2'-O-methyl uridine 3'-phosphodiester
  modified_base 6: mod_base = OTHER; note = 2'-deoxyguanosine 3'-phosphodiester
  modified_base 7: mod_base = OTHER; note = 2'-O-methyl adenosine 3'-phosphodiester
  modified_base 8: mod_base = OTHER; note = 2'-O-methyl guanosine 3'-phosphodiester
  modified_base 9: mod_base = OTHER; note = 2'-O-methoxyethyl adenosine 3'-phosphodiester
  modified_base 10: mod_base = OTHER; note = 2'-O-methyl adenosine 3'-phosphodiester
  modified_base 11: mod_base = OTHER; note = 2'-O-methyl uridine 3'-phosphodiester
  modified_base 12: mod_base = OTHER; note = 2'-O-methyl adenosine 3'-phosphodiester
  modified_base 13: mod_base = OTHER; note = 2'-O-methyl cytidine 3'-phosphodiester
  modified_base 14: mod_base = OTHER; note = 2'-fluoro uridine 3'-phosphodiester
  modified_base 15: mod_base = OTHER; note = 2'-O-methyl guanosine 3'-phosphodiester
  modified_base 16: mod_base = OTHER; note = 2'-fluoro uridine 3'-phosphodiester
  modified_base 17..19: mod_base = OTHER; note = 2'-O-methyl cytidine 3'-phosphodiester
  modified_base 20: mod_base = OTHER; note = 2'-O-methyl uridine 3'-phosphodiester
  modified_base 21: mod_base = OTHER; note = 2'-O-methyl uridine 3'-phosphorothioate
  modified_base 22: mod_base = OTHER; note = 2'-O-methoxyethyl adenosine 3'-phosphorothioate
  modified_base 23: mod_base = OTHER; note = 2'-O-methoxyethyl adenosine
tcactgagaatactgtcccttaa

SEQ ID NO: 68 - RNA, 23 nt, synthetic construct
  source 1..23: mol_type = other RNA
  modified_base 1: mod_base = OTHER; note = 2'-O-methoxyethyl thymidine 3'-phosphorothioate
  modified_base 2: mod_base = OTHER; note = 2'-fluoro cytidine 3'-phosphorothioate
  modified_base 3: mod_base = OTHER; note = 2'-O-methyl adenosine 3'-phosphodiester
  modified_base 4: mod_base = OTHER; note = 2'-O-methyl cytidine 3'-phosphodiester
  modified_base 5: mod_base = OTHER; note = 2'-O-methyl uridine 3'-phosphodiester
  modified_base 6: mod_base = OTHER; note = 2'-deoxyguanosine 3'-phosphodiester
  modified_base 7: mod_base = OTHER; note = 2'-O-methyl adenosine 3'-phosphodiester
  modified_base 8: mod_base = OTHER; note = 2'-O-methyl guanosine 3'-phosphodiester
  modified_base 9: mod_base = OTHER; note = 2'-O-methoxyethyl adenosine 3'-phosphodiester
  modified_base 10: mod_base = OTHER; note = 2'-O-methyl adenosine 3'-phosphodiester
  modified_base 11: mod_base = OTHER; note = 2'-O-methyl uridine 3'-phosphodiester
  modified_base 12: mod_base = OTHER; note = 2'-O-methyl adenosine 3'-phosphodiester
  modified_base 13: mod_base = OTHER; note = 2'-O-methyl cytidine 3'-phosphodiester
  modified_base 14: mod_base = OTHER; note = 2'-deoxythymidine 3'-phosphodiester
  modified_base 15: mod_base = OTHER; note = 2'-O-methyl guanosine 3'-phosphodiester
  modified_base 16: mod_base = OTHER; note = 2'-deoxythymidine 3'-phosphodiester
  modified_base 17..19: mod_base = OTHER; note = 2'-O-methyl cytidine 3'-phosphodiester
  modified_base 20: mod_base = OTHER; note = 2'-O-methyl uridine 3'-phosphodiester
  modified_base 21: mod_base = OTHER; note = 2'-O-methyl uridine 3'-phosphorothioate
  modified_base 22: mod_base = OTHER; note = 2'-O-methoxyethyl adenosine 3'-phosphorothioate
  modified_base 23: mod_base = OTHER; note = 2'-O-methoxyethyl adenosine
tcactgagaatactgtcccttaa

SEQ ID NO: 69 - RNA, 23 nt, synthetic construct
  source 1..23: mol_type = other RNA
  modified_base 1: mod_base = OTHER; note = 2'-O-methoxyethyl thymidine 3'-phosphorothioate
  modified_base 2: mod_base = OTHER; note = 2'-fluoro cytidine 3'-phosphorothioate
  modified_base 3: mod_base = OTHER; note = 2'-O-methyl adenosine 3'-phosphodiester
  modified_base 4: mod_base = OTHER; note = 2'-O-methyl cytidine 3'-phosphodiester
  modified_base 5: mod_base = OTHER; note = 2'-O-methyl cytidine 3'-phosphodiester
  modified_base 6: mod_base = OTHER; note = 2'-deoxyguanosine 3'-phosphorothioate
  modified_base 7: mod_base = OTHER; note = 2'-O-methyl adenosine 3'-phosphodiester
  modified_base 8: mod_base = OTHER; note = 2'-O-methyl guanosine 3'-phosphodiester
  modified_base 9: mod_base = OTHER; note = 2'-O-methoxyethyl adenosine 3'-phosphodiester
  modified_base 10: mod_base = OTHER; note = 2'-O-methyl adenosine 3'-phosphodiester
  modified_base 11: mod_base = OTHER; note = 2'-O-methyl uridine 3'-phosphodiester
  modified_base 12: mod_base = OTHER; note = 2'-O-methyl adenosine 3'-phosphodiester
  modified_base 13: mod_base = OTHER; note = 2'-O-methyl cytidine 3'-phosphodiester
  modified_base 14: mod_base = OTHER; note = 2'-deoxyuridine 3'-phosphorothioate
  modified_base 15: mod_base = OTHER; note = 2'-O-methyl guanosine 3'-phosphodiester
  modified_base 16: mod_base = OTHER; note = 2'-deoxyuridine 3'-phosphorothioate
  modified_base 17..19: mod_base = OTHER; note = 2'-O-methyl cytidine 3'-phosphodiester
  modified_base 20: mod_base = OTHER; note = 2'-O-methyl uridine 3'-phosphodiester
  modified_base 21: mod_base = OTHER; note = 2'-O-methyl uridine 3'-phosphorothioate
  modified_base 22: mod_base = OTHER; note = 2'-O-methoxyethyl adenosine 3'-phosphorothioate
  modified_base 23: mod_base = OTHER; note = 2'-O-methoxyethyl adenosine
tcactgagaatactgtcccttaa

SEQ ID NO: 70 - RNA, 23 nt, synthetic construct
  source 1..23: mol_type = other RNA
  modified_base 1: mod_base = OTHER; note = 2'-O-methoxyethyl thymidine 3'-phosphorothioate
  modified_base 2: mod_base = OTHER; note = 2'-fluoro cytidine 3'-phosphorothioate
  modified_base 3: mod_base = OTHER; note = 2'-O-methyl adenosine 3'-phosphodiester
  modified_base 4: mod_base = OTHER; note = 2'-O-methyl cytidine 3'-phosphodiester
  modified_base 5: mod_base = OTHER; note = 2'-O-methyl uridine 3'-phosphodiester
  modified_base 6: mod_base = OTHER; note = 2'-deoxyguanosine 3'-phosphodiester
  modified_base 7: mod_base = OTHER; note = 2'-O-methyl adenosine 3'-phosphodiester
  modified_base 8: mod_base = OTHER; note = 2'-O-methyl guanosine 3'-phosphodiester
  modified_base 9..10: mod_base = OTHER; note = 2'-O-methoxyethyl adenosine 3'-phosphodiester
  modified_base 11: mod_base = OTHER; note = 2'-O-methyl uridine 3'-phosphodiester
  modified_base 12: mod_base = OTHER; note = 2'-O-methyl adenosine 3'-phosphodiester
  modified_base 13: mod_base = OTHER; note = 2'-O-methyl cytidine 3'-phosphodiester
  modified_base 14: mod_base = OTHER; note = 2'-fluoro uridine 3'-phosphodiester
  modified_base 15: mod_base = OTHER; note = 2'-O-methyl guanosine 3'-phosphodiester
  modified_base 16: mod_base = OTHER; note = 2'-fluoro uridine 3'-phosphodiester
  modified_base 17..19: mod_base = OTHER; note = 2'-O-methyl cytidine 3'-phosphodiester
  modified_base 20: mod_base = OTHER; note = 2'-O-methyl uridine 3'-phosphodiester
  modified_base 21: mod_base = OTHER; note = 2'-O-methyl uridine 3'-phosphorothioate
  modified_base 22: mod_base = OTHER; note = 2'-O-methoxyethyl adenosine 3'-phosphorothioate
  modified_base 23: mod_base = OTHER; note = 2'-O-methoxyethyl adenosine
tcactgagaatactgtcccttaa

SEQ ID NO: 71 - RNA, 23 nt, synthetic construct
  source 1..23: mol_type = other RNA
  modified_base 1: mod_base = OTHER; note = 2'-O-methoxyethyl thymidine 3'-phosphorothioate
  modified_base 2: mod_base = OTHER; note = 2'-fluoro cytidine 3'-phosphorothioate
  modified_base 3: mod_base = OTHER; note = 2'-O-methyl adenosine 3'-phosphodiester
  modified_base 4: mod_base = OTHER; note = 2'-O-methyl cytidine 3'-phosphodiester
  modified_base 5: mod_base = OTHER; note = 2'-O-methyl uridine 3'-phosphodiester
  modified_base 6: mod_base = OTHER; note = 2'-deoxyguanosine 3'-phosphorothioate
  modified_base 7: mod_base = OTHER; note = 2'-O-methyl adenosine 3'-phosphodiester
  modified_base 8: mod_base = OTHER; note = 2'-O-methyl guanosine 3'-phosphodiester
  modified_base 9: mod_base = OTHER; note = 2'-O-methoxyethyl adenosine 3'-phosphodiester
  modified_base 10: mod_base = OTHER; note = 2'-O-methyl adenosine 3'-phosphodiester
  modified_base 11: mod_base = OTHER; note = 2'-O-methyl uridine 3'-phosphodiester
  modified_base 12: mod_base = OTHER; note = 2'-O-methyl adenosine 3'-phosphodiester
  modified_base 13: mod_base = OTHER; note = 2'-O-methyl cytidine 3'-phosphodiester
  modified_base 14: mod_base = OTHER; note = 2'-deoxythymidine 3'-phosphorothioate
  modified_base 15: mod_base = OTHER; note = 2'-O-methyl guanosine 3'-phosphodiester
  modified_base 16: mod_base = OTHER; note = 2'-deoxythymidine 3'-phosphorothioate
  modified_base 17..19: mod_base = OTHER; note = 2'-O-methyl cytidine 3'-phosphodiester
  modified_base 20: mod_base = OTHER; note = 2'-O-methyl uridine 3'-phosphodiester
  modified_base 21: mod_base = OTHER; note = 2'-O-methyl uridine 3'-phosphorothioate
  modified_base 22: mod_base = OTHER; note = 2'-O-methoxyethyl adenosine 3'-phosphorothioate
  modified_base 23: mod_base = OTHER; note = 2'-O-methoxyethyl adenosine
tcactgagaatactgtcccttaa

SEQ ID NO: 72 - RNA, 21 nt, synthetic construct
  source 1..21: mol_type = other RNA
  modified_base 1: mod_base = OTHER; note = 2'-O-methoxyethyl thymidine 3'-phosphorothioate
  modified_base 2: mod_base = OTHER; note = 2'-fluoro cytidine 3'-phosphorothioate
  modified_base 3: mod_base = OTHER; note = 2'-O-methyl adenosine 3'-phosphodiester
  modified_base 4: mod_base = OTHER; note = 2'-O-methyl cytidine 3'-phosphodiester
  modified_base 5: mod_base = OTHER; note = 2'-O-methyl uridine 3'-phosphodiester
  modified_base 6: mod_base = OTHER; note = 2'-fluoro guanosine 3'-phosphodiester
  modified_base 7: mod_base = OTHER; note = 2'-O-methyl adenosine 3'-phosphodiester
  modified_base 8: mod_base = OTHER; note = 2'-O-methyl guanosine 3'-phosphodiester
  modified_base 9..10: mod_base = OTHER; note = 2'-O-methyl adenosine 3'-phosphodiester
  modified_base 11: mod_base = OTHER; note = 2'-O-methyl uridine 3'-phosphodiester
  modified_base 12: mod_base = OTHER; note = 2'-O-methyl adenosine 3'-phosphodiester
  modified_base 13: mod_base = OTHER; note = 2'-O-methyl cytidine 3'-phosphodiester
  modified_base 14: mod_base = OTHER; note = 2'-fluoro uridine 3'-phosphodiester
  modified_base 15: mod_base = OTHER; note = 2'-O-methyl guanosine 3'-phosphodiester
  modified_base 16: mod_base = OTHER; note = 2'-fluoro uridine 3'-phosphodiester
  modified_base 17..18: mod_base = OTHER; note = 2'-O-methyl cytidine 3'-phosphodiester
  modified_base 19: mod_base = OTHER; note = 2'-O-methyl cytidine 3'-phosphorothioate
  modified_base 20: mod_base = OTHER; note = 2'-O-methoxyethyl adenosine 3'-phosphorothioate
  modified_base 21: mod_base = OTHER; note = 2'-O-methoxyethyl adenosine
tcactgagaatactgtcccaa

SEQ ID NO: 73 - RNA, 21 nt, synthetic construct
  source 1..21: mol_type = other RNA
  modified_base 1: mod_base = OTHER; note = 2'-O-methoxyethyl thymidine 3'-phosphorothioate
  modified_base 2: mod_base = OTHER; note = 2'-fluoro cytidine 3'-phosphorothioate
  modified_base 3: mod_base = OTHER; note = 2'-O-methyl adenosine 3'-phosphodiester
  modified_base 4: mod_base = OTHER; note = 2'-O-methyl cytidine 3'-phosphodiester
  modified_base 5: mod_base = OTHER; note = 2'-O-methyl uridine 3'-phosphodiester
  modified_base 6: mod_base = OTHER; note = 2'-deoxyguanosine 3'-phosphorothioate
  modified_base 7: mod_base = OTHER; note = 2'-O-methyl adenosine 3'-phosphodiester
  modified_base 8: mod_base = OTHER; note = 2'-O-methyl guanosine 3'-phosphodiester
  modified_base 9..10: mod_base = OTHER; note = 2'-O-methyl adenosine 3'-phosphodiester
  modified_base 11: mod_base = OTHER; note = 2'-O-methyl uridine 3'-phosphodiester
  modified_base 12: mod_base = OTHER; note = 2'-O-methyl adenosine 3'-phosphodiester
  modified_base 13: mod_base = OTHER; note = 2'-O-methyl cytidine 3'-phosphodiester
  modified_base 14: mod_base = OTHER; note = 2'-fluoro uridine 3'-phosphodiester
  modified_base 15: mod_base = OTHER; note = 2'-O-methyl guanosine 3'-phosphodiester
  modified_base 16: mod_base = OTHER; note = 2'-fluoro uridine 3'-phosphodiester
  modified_base 17..18: mod_base = OTHER; note = 2'-O-methyl cytidine 3'-phosphodiester
  modified_base 19: mod_base = OTHER; note = 2'-O-methyl cytidine 3'-phosphorothioate
  modified_base 20: mod_base = OTHER; note = 2'-O-methoxyethyl adenosine 3'-phosphorothioate
  modified_base 21: mod_base = OTHER; note = 2'-O-methoxyethyl adenosine
tcactgagaatactgtcccaa

SEQ ID NO: 74 - RNA, 21 nt, synthetic construct
  source 1..21: mol_type = other RNA
  modified_base 1: mod_base = OTHER; note = 2'-O-methoxyethyl thymidine 3'-phosphorothioate
  modified_base 2: mod_base = OTHER; note = 2'-fluoro cytidine 3'-phosphorothioate
  modified_base 3: mod_base = OTHER; note = 2'-O-methyl adenosine 3'-phosphodiester
  modified_base 4: mod_base = OTHER; note = 2'-O-methyl cytidine 3'-phosphodiester
  modified_base 5: mod_base = OTHER; note = 2'-O-methyl uridine 3'-phosphodiester
  modified_base 6: mod_base = OTHER; note = 2'-deoxyguanosine 3'-phosphorothioate
  modified_base 7: mod_base = OTHER; note = 2'-O-methyl adenosine 3'-phosphodiester
  modified_base 8: mod_base = OTHER; note = 2'-O-methyl guanosine 3'-phosphodiester
  modified_base 9..10: mod_base = OTHER; note = 2'-O-methyl adenosine 3'-phosphodiester
  modified_base 11: mod_base = OTHER; note = 2'-O-methyl uridine 3'-phosphodiester
  modified_base 12: mod_base = OTHER; note = 2'-O-methyl adenosine 3'-phosphodiester
  modified_base 13: mod_base = OTHER; note = 2'-O-methyl cytidine 3'-phosphodiester
  modified_base 14: mod_base = OTHER; note = 2'-deoxyuridine 3'-phosphorothioate
  modified_base 15: mod_base = OTHER; note = 2'-O-methyl guanosine 3'-phosphodiester
  modified_base 16: mod_base = OTHER; note = 2'-deoxyuridine 3'-phosphorothioate
  modified_base 17..18: mod_base = OTHER; note = 2'-O-methyl cytidine 3'-phosphodiester
  modified_base 19: mod_base = OTHER; note = 2'-O-methyl cytidine 3'-phosphorothioate
  modified_base 20: mod_base = OTHER; note = 2'-O-methoxyethyl adenosine 3'-phosphorothioate
  modified_base 21: mod_base = OTHER; note = 2'-O-methoxyethyl adenosine
tcactgagaatactgtcccaa

SEQ ID NO: 75 - RNA, 21 nt, synthetic construct
  source 1..21: mol_type = other RNA
  modified_base 1..2: mod_base = OTHER; note = 2'-O-methyl adenosine 3'-phosphorothioate
  modified_base 3..5: mod_base = OTHER; note = 2'-O-methyl guanosine 3'-phosphodiester
  modified_base 6: mod_base = OTHER; note = 2'-O-methyl adenosine 3'-phosphodiester
  modified_base 7: mod_base = OTHER; note = 2'-fluoro cytidine 3'-phosphodiester
  modified_base 8: mod_base = OTHER; note = 2'-O-methyl adenosine 3'-phosphodiester
  modified_base 9: mod_base = OTHER; note = 2'-fluoro guanosine 3'-phosphodiester
  modified_base 10: mod_base = OTHER; note = 2'-fluoro uridine 3'-phosphodiester
  modified_base 11: mod_base = OTHER; note = 2'-fluoro adenosine 3'-phosphodiester
  modified_base 12..13: mod_base = OTHER; note = 2'-O-methyl uridine 3'-phosphodiester
  modified_base 15: mod_base = OTHER; note = 2'-O-methyl uridine 3'-phosphodiester
  modified_base 14: mod_base = OTHER; note = 2'-O-methyl cytidine 3'-phosphodiester
  modified_base 16: mod_base = OTHER; note = 2'-O-methyl cytidine 3'-phosphodiester
  modified_base 17: mod_base = OTHER; note = 2'-O-methyl adenosine 3'-phosphodiester
  modified_base 18: mod_base = OTHER; note = 2'-O-methyl guanosine 3'-phosphodiester
  modified_base 19: mod_base = OTHER; note = 2'-O-methyl uridine 3'-phosphorothioate
  modified_base 20: mod_base = OTHER; note = 2'-O-methyl guanosine 3'-phosphorothioate
  modified_base 21: mod_base = OTHER; note = 2'-O-methyl adenosine
aagggacagtattctcagtga

SEQ ID NO: 76 - RNA, 21 nt, synthetic construct
  source 1..21: mol_type = other RNA
  modified_base 1..2: mod_base = OTHER; note = 2'-O-methyl adenosine 3'-phosphorothioate
  modified_base 3..5: mod_base = OTHER; note = 2'-O-methyl guanosine 3'-phosphodiester
  modified_base 6: mod_base = OTHER; note = 2'-O-methyl adenosine 3'-phosphodiester
  modified_base 7: mod_base = OTHER; note = 2'-O-methyl cytidine 3'-phosphodiester
  modified_base 8: mod_base = OTHER; note = 2'-O-methyl adenosine 3'-phosphodiester
  modified_base 9: mod_base = OTHER; note = 2'-O-methyl guanosine 3'-phosphodiester
  modified_base 10: mod_base = OTHER; note = 2'-fluoro uridine 3'-phosphodiester
  modified_base 11: mod_base = OTHER; note = 2'-fluoro adenosine 3'-phosphodiester
  modified_base 12..13: mod_base = OTHER; note = 2'-O-methyl uridine 3'-phosphodiester
  modified_base 14: mod_base = OTHER; note = 2'-O-methyl cytidine 3'-phosphodiester
  modified_base 15: mod_base = OTHER; note = 2'-O-methyl uridine 3'-phosphodiester
  modified_base 16: mod_base = OTHER; note = 2'-O-methyl cytidine 3'-phosphodiester
  modified_base 17: mod_base = OTHER; note = 2'-O-methyl adenosine 3'-phosphodiester
  modified_base 18: mod_base = OTHER; note = 2'-O-methyl guanosine 3'-phosphodiester
  modified_base 19: mod_base = OTHER; note = 2'-O-methyl uridine 3'-phosphorothioate
  modified_base 20: mod_base = OTHER; note = 2'-O-methyl guanosine 3'-phosphorothioate
  modified_base 21: mod_base = OTHER; note = 2'-O-methyl adenosine
aagggacagtattctcagtga

SEQ ID NO: 77 - RNA, 21 nt, synthetic construct
  source 1..21: mol_type = other RNA
  modified_base 1..2: mod_base = OTHER; note = 2'-O-methyl adenosine 3'-phosphorothioate
  modified_base 3..5: mod_base = OTHER; note = 2'-O-methyl guanosine 3'-phosphodiester
  modified_base 6: mod_base = OTHER; note = 2'-O-methyl adenosine 3'-phosphodiester
  modified_base 7: mod_base = OTHER; note = 2'-O-methyl cytidine 3'-phosphodiester
  modified_base 8: mod_base = OTHER; note = 2'-fluoro adenosine 3'-phosphodiester
  modified_base 9: mod_base = OTHER; note = 2'-O-methyl guanosine 3'-phosphodiester
  modified_base 10: mod_base = OTHER; note = 2'-fluoro uridine 3'-phosphodiester
  modified_base 11: mod_base = OTHER; note = 2'-O-methyl adenosine 3'-phosphodiester
  modified_base 12: mod_base = OTHER; note = 2'-fluoro uridine 3'-phosphodiester
  modified_base 13: mod_base = OTHER; note = 2'-O-methyl uridine 3'-phosphodiester
  modified_base 14: mod_base = OTHER; note = 2'-O-methyl cytidine 3'-phosphodiester
  modified_base 15: mod_base = OTHER; note = 2'-O-methyl uridine 3'-phosphodiester
  modified_base 16: mod_base = OTHER; note = 2'-O-methyl cytidine 3'-phosphodiester
  modified_base 17: mod_base = OTHER; note = 2'-O-methyl adenosine 3'-phosphodiester
  modified_base 18: mod_base = OTHER; note = 2'-O-methyl guanosine 3'-phosphodiester
  modified_base 19: mod_base = OTHER; note = 2'-O-methyl uridine 3'-phosphorothioate
  modified_base 20: mod_base = OTHER; note = 2'-O-methyl guanosine 3'-phosphorothioate
  modified_base 21: mod_base = OTHER; note = 2'-O-methyl adenosine
aagggacagtattctcagtga

SEQ ID NO: 78 - RNA, 21 nt, synthetic construct
  source 1..21: mol_type = other RNA
  modified_base 1..2: mod_base = OTHER; note = 2'-O-methyl adenosine 3'-phosphorothioate
  modified_base 3..5: mod_base = OTHER; note = 2'-O-methyl guanosine 3'-phosphodiester
  modified_base 6: mod_base = OTHER; note = 2'-O-methyl adenosine 3'-phosphodiester
  modified_base 7: mod_base = OTHER; note = 2'-O-methyl cytidine 3'-phosphodiester
  modified_base 8: mod_base = OTHER; note = 2'-O-methyl adenosine 3'-phosphodiester
  modified_base 9: mod_base = OTHER; note = 2'-O-methyl guanosine 3'-phosphodiester
  modified_base 10: mod_base = OTHER; note = 2'-fluoro uridine 3'-phosphorothioate
  modified_base 11: mod_base = OTHER; note = 2'-fluoro adenosine 3'-phosphodiester
  modified_base 12..13: mod_base = OTHER; note = 2'-O-methyl uridine 3'-phosphodiester
  modified_base 14: mod_base = OTHER; note = 2'-O-methyl cytidine 3'-phosphodiester
  modified_base 15: mod_base = OTHER; note = 2'-O-methyl uridine 3'-phosphodiester
  modified_base 16: mod_base = OTHER; note = 2'-O-methyl cytidine 3'-phosphodiester
  modified_base 17: mod_base = OTHER; note = 2'-O-methyl adenosine 3'-phosphodiester
  modified_base 18: mod_base = OTHER; note = 2'-O-methyl guanosine 3'-phosphodiester
  modified_base 19: mod_base = OTHER; note = 2'-O-methyl uridine 3'-phosphorothioate
  modified_base 20: mod_base = OTHER; note = 2'-O-methyl guanosine 3'-phosphorothioate
  modified_base 21: mod_base = OTHER; note = 2'-O-methyl adenosine
aagggacagtattctcagtga

SEQ ID NO: 79 - RNA, 21 nt, synthetic construct
  source 1..21: mol_type = other RNA
  modified_base 1..2: mod_base = OTHER; note = 2'-O-methoxyethyl adenosine 3'-phosphorothioate
  modified_base 3..5: mod_base = OTHER; note = 2'-O-methyl guanosine 3'-phosphodiester
  modified_base 6: mod_base = OTHER; note = 2'-O-methyl adenosine 3'-phosphodiester
  modified_base 7: mod_base = OTHER; note = 2'-O-methyl cytidine 3'-phosphodiester
  modified_base 8: mod_base = OTHER; note = 2'-O-methyl adenosine 3'-phosphodiester
  modified_base 9: mod_base = OTHER; note = 2'-O-methyl guanosine 3'-phosphodiester
  modified_base 10: mod_base = OTHER; note = 2'-fluoro uridine 3'-phosphodiester
  modified_base 11: mod_base = OTHER; note = 2'-fluoro adenosine 3'-phosphodiester
  modified_base 12..13: mod_base = OTHER; note = 2'-O-methyl uridine 3'-phosphodiester
  modified_base 14: mod_base = OTHER; note = 2'-O-methyl cytidine 3'-phosphodiester
  modified_base 15: mod_base = OTHER; note = 2'-O-methyl uridine 3'-phosphodiester
  modified_base 16: mod_base = OTHER; note = 2'-O-methyl cytidine 3'-phosphodiester
  modified_base 17: mod_base = OTHER; note = 2'-O-methyl adenosine 3'-phosphodiester
  modified_base 18: mod_base = OTHER; note = 2'-O-methyl guanosine 3'-phosphodiester
  modified_base 19: mod_base = OTHER; note = 2'-O-methyl uridine 3'-phosphorothioate
  modified_base 20: mod_base = OTHER; note = 2'-O-methoxyethyl guanosine 3'-phosphorothioate
  modified_base 21: mod_base = OTHER; note = 2'-O-methoxyethyl adenosine
aagggacagtattctcagtga

SEQ ID NO: 80 - RNA, 21 nt, synthetic construct
  source 1..21: mol_type = other RNA
  modified_base 1..2: mod_base = OTHER; note = 2'-O-methoxyethyl adenosine 3'-phosphorothioate
  modified_base 3..5: mod_base = OTHER; note = 2'-O-methyl guanosine 3'-phosphodiester
  modified_base 6: mod_base = OTHER; note = 2'-O-methyl adenosine 3'-phosphodiester
  modified_base 7: mod_base = OTHER; note = 2'-O-methyl cytidine 3'-phosphodiester
  modified_base 8: mod_base = OTHER; note = 2'-O-methyl adenosine 3'-phosphodiester
  modified_base 9: mod_base = OTHER; note = 2'-O-methyl guanosine 3'-phosphodiester
  modified_base 10: mod_base = OTHER; note = 2'-fluoro uridine 3'-phosphorothioate
  modified_base 11: mod_base = OTHER; note = 2'-fluoro adenosine 3'-phosphodiester
  modified_base 12..13: mod_base = OTHER; note = 2'-O-methyl uridine 3'-phosphodiester
  modified_base 14: mod_base = OTHER; note = 2'-O-methyl cytidine 3'-phosphodiester
  modified_base 15: mod_base = OTHER; note = 2'-O-methyl uridine 3'-phosphodiester
  modified_base 16: mod_base = OTHER; note = 2'-O-methyl cytidine 3'-phosphodiester
  modified_base 17: mod_base = OTHER; note = 2'-O-methyl adenosine 3'-phosphodiester
  modified_base 18: mod_base = OTHER; note = 2'-O-methyl guanosine 3'-phosphodiester
  modified_base 19: mod_base = OTHER; note = 2'-O-methyl uridine 3'-phosphorothioate
  modified_base 20: mod_base = OTHER; note = 2'-O-methoxyethyl guanosine 3'-phosphorothioate
  modified_base 21: mod_base = OTHER; note = 2'-O-methoxyethyl adenosine
aagggacagtattctcagtga

SEQ ID NO: 81 - RNA, 21 nt, synthetic construct
  source 1..21: mol_type = other RNA
  modified_base 1..2: mod_base = OTHER; note = 2'-O-methoxyethyl adenosine 3'-phosphorothioate
  modified_base 3..5: mod_base = OTHER; note = 2'-O-methyl guanosine 3'-phosphodiester
  modified_base 6: mod_base = OTHER; note = 2'-O-methyl adenosine 3'-phosphodiester
  modified_base 7: mod_base = OTHER; note = 2'-O-methyl cytidine 3'-phosphodiester
  modified_base 8: mod_base = OTHER; note = 2'-O-methyl adenosine 3'-phosphodiester
  modified_base 9: mod_base = OTHER; note = 2'-fluoro guanosine 3'-phosphorothioate
  modified_base 10: mod_base = OTHER; note = 2'-fluoro uridine 3'-phosphodiester
  modified_base 11: mod_base = OTHER; note = 2'-O-methyl adenosine 3'-phosphodiester
  modified_base 12..13: mod_base = OTHER; note = 2'-O-methyl uridine 3'-phosphodiester
  modified_base 14: mod_base = OTHER; note = 2'-O-methyl cytidine 3'-phosphodiester
  modified_base 15: mod_base = OTHER; note = 2'-O-methyl uridine 3'-phosphodiester
  modified_base 16: mod_base = OTHER; note = 2'-O-methyl cytidine 3'-phosphodiester
  modified_base 17: mod_base = OTHER; note = 2'-O-methyl adenosine 3'-phosphodiester
  modified_base 18: mod_base = OTHER; note = 2'-O-methyl guanosine 3'-phosphodiester
  modified_base 19: mod_base = OTHER; note = 2'-O-methyl uridine 3'-phosphorothioate
  modified_base 20: mod_base = OTHER; note = 2'-O-methoxyethyl guanosine 3'-phosphorothioate
  modified_base 21: mod_base = OTHER; note = 2'-O-methoxyethyl adenosine
aagggacagtattctcagtga

SEQ ID NO: 82 - RNA, 19 nt, synthetic construct
  source 1..19: mol_type = other RNA
  modified_base 1..2: mod_base = OTHER; note = 2'-O-methyl guanosine 3'-phosphorothioate
  modified_base 3: mod_base = OTHER; note = 2'-O-methyl guanosine 3'-phosphodiester
  modified_base 4: mod_base = OTHER; note = 2'-O-methyl adenosine 3'-phosphodiester
  modified_base 5: mod_base = OTHER; note = 2'-O-methyl cytidine 3'-phosphodiester
  modified_base 6: mod_base = OTHER; note = 2'-O-methyl adenosine 3'-phosphodiester
  modified_base 7: mod_base = OTHER; note = 2'-fluoro guanosine 3'-phosphodiester
  modified_base 8: mod_base = OTHER; note = 2'-O-methyl uridine 3'-phosphodiester
  modified_base 9: mod_base = OTHER; note = 2'-fluoro adenosine 3'-phosphodiester
  modified_base 10..11: mod_base = OTHER; note = 2'-fluoro uridine 3'-phosphodiester
  modified_base 12: mod_base = OTHER; note = 2'-O-methyl cytidine 3'-phosphodiester
  modified_base 13: mod_base = OTHER; note = 2'-O-methyl uridine 3'-phosphodiester
  modified_base 14: mod_base = OTHER; note = 2'-O-methyl cytidine 3'-phosphodiester
  modified_base 15: mod_base = OTHER; note = 2'-O-methyl adenosine 3'-phosphodiester
  modified_base 16: mod_base = OTHER; note = 2'-O-methyl guanosine 3'-phosphodiester
  modified_base 17: mod_base = OTHER; note = 2'-O-methyl uridine 3'-phosphorothioate
  modified_base 18: mod_base = OTHER; note = 2'-O-methyl guanosine 3'-phosphorothioate
  modified_base 19: mod_base = OTHER; note = 2'-O-methyl adenosine
gggacagtattctcagtga

SEQ ID NO: 83 - RNA, 19 nt, synthetic construct
  source 1..19: mol_type = other RNA
  modified_base 1..2: mod_base = OTHER; note = 2'-O-methyl guanosine 3'-phosphorothioate
  modified_base 3: mod_base = OTHER; note = 2'-O-methyl guanosine 3'-phosphodiester
  modified_base 4: mod_base = OTHER; note = 2'-O-methyl adenosine 3'-phosphodiester
  modified_base 5: mod_base = OTHER; note = 2'-O-methyl cytidine 3'-phosphodiester
  modified_base 6: mod_base = OTHER; note = 2'-O-methyl adenosine 3'-phosphodiester
  modified_base 7: mod_base = OTHER; note = 2'-fluoro guanosine 3'-phosphodiester
  modified_base 8: mod_base = OTHER; note = 2'-O-methyl uridine 3'-phosphodiester
  modified_base 9: mod_base = OTHER; note = 2'-fluoro adenosine 3'-phosphodiester
  modified_base 10..11: mod_base = OTHER; note = 2'-O-methyl uridine 3'-phosphodiester
  modified_base 12: mod_base = OTHER; note = 2'-O-methyl cytidine 3'-phosphodiester
  modified_base 13: mod_base = OTHER; note = 2'-O-methyl uridine 3'-phosphodiester
  modified_base 14: mod_base = OTHER; note = 2'-O-methyl cytidine 3'-phosphodiester
  modified_base 15: mod_base = OTHER; note = 2'-O-methyl adenosine 3'-phosphodiester
  modified_base 16: mod_base = OTHER; note = 2'-O-methyl guanosine 3'-phosphodiester
  modified_base 17: mod_base = OTHER; note = 2'-O-methyl uridine 3'-phosphorothioate
  modified_base 18: mod_base = OTHER; note = 2'-O-methyl guanosine 3'-phosphorothioate
  modified_base 19: mod_base = OTHER; note = 2'-O-methyl adenosine
gggacagtattctcagtga

SEQ ID NO: 84 - RNA, 19 nt, synthetic construct
  source 1..19: mol_type = other RNA
  modified_base 1..2: mod_base = OTHER; note = 2'-O-methyl guanosine 3'-phosphorothioate
  modified_base 3: mod_base = OTHER; note = 2'-O-methyl guanosine 3'-phosphodiester
  modified_base 4: mod_base = OTHER; note = 2'-O-methyl adenosine 3'-phosphodiester
  modified_base 5: mod_base = OTHER; note = 2'-O-methyl cytidine 3'-phosphodiester
  modified_base 6: mod_base = OTHER; note = 2'-O-methyl adenosine 3'-phosphodiester
  modified_base 7: mod_base = OTHER; note = 2'-O-methyl guanosine 3'-phosphodiester
  modified_base 8: mod_base = OTHER; note = 2'-fluoro uridine 3'-phosphodiester
  modified_base 9: mod_base = OTHER; note = 2'-O-methyl adenosine 3'-phosphodiester
  modified_base 10: mod_base = OTHER; note = 2'-fluoro uridine 3'-phosphodiester
  modified_base 11: mod_base = OTHER; note = 2'-O-methyl uridine 3'-phosphodiester
  modified_base 12: mod_base = OTHER; note = 2'-O-methyl cytidine 3'-phosphodiester
  modified_base 13: mod_base = OTHER; note = 2'-O-methyl uridine 3'-phosphodiester
  modified_base 14: mod_base = OTHER; note = 2'-O-methyl cytidine 3'-phosphodiester
  modified_base 15: mod_base = OTHER; note = 2'-O-methyl adenosine 3'-phosphodiester
  modified_base 16: mod_base = OTHER; note = 2'-O-methyl guanosine 3'-phosphodiester
  modified_base 17: mod_base = OTHER; note = 2'-O-methyl uridine 3'-phosphorothioate
  modified_base 18: mod_base = OTHER; note = 2'-O-methyl guanosine 3'-phosphorothioate
  modified_base 19: mod_base = OTHER; note = 2'-O-methyl adenosine
gggacagtattctcagtga

SEQ ID NO: 85 - RNA, 19 nt, synthetic construct
  source 1..19: mol_type = other RNA
  modified_base 1..2: mod_base = OTHER; note = 2'-O-methyl guanosine 3'-phosphorothioate
  modified_base 3: mod_base = OTHER; note = 2'-O-methyl guanosine 3'-phosphodiester
  modified_base 4: mod_base = OTHER; note = 2'-O-methyl adenosine 3'-phosphodiester
  modified_base 5: mod_base = OTHER; note = 2'-O-methyl cytidine 3'-phosphodiester
  modified_base 6: mod_base = OTHER; note = 2'-O-methyl adenosine 3'-phosphodiester
  modified_base 7: mod_base = OTHER; note = 2'-O-methyl guanosine 3'-phosphodiester
  modified_base 8: mod_base = OTHER; note = 2'-O-methyl uridine 3'-phosphodiester
  modified_base 9: mod_base = OTHER; note = 2'-fluoro adenosine 3'-phosphodiester
  modified_base 10: mod_base = OTHER; note = 2'-O-methyl uridine 3'-phosphodiester
  modified_base 11: mod_base = OTHER; note = 2'-fluoro uridine 3'-phosphodiester
  modified_base 12: mod_base = OTHER; note = 2'-O-methyl cytidine 3'-phosphodiester
  modified_base 13: mod_base = OTHER; note = 2'-O-methyl uridine 3'-phosphodiester
  modified_base 14: mod_base = OTHER; note = 2'-O-methyl cytidine 3'-phosphodiester
  modified_base 15: mod_base = OTHER; note = 2'-O-methyl adenosine 3'-phosphodiester
  modified_base 16: mod_base = OTHER; note = 2'-O-methyl guanosine 3'-phosphodiester
  modified_base 17: mod_base = OTHER; note = 2'-O-methyl uridine 3'-phosphorothioate
  modified_base 18: mod_base = OTHER; note = 2'-O-methyl guanosine 3'-phosphorothioate
  modified_base 19: mod_base = OTHER; note = 2'-O-methyl adenosine
gggacagtattctcagtga

SEQ ID NO: 86 - RNA, 19 nt, synthetic construct
  source 1..19: mol_type = other RNA
  modified_base 1..2: mod_base = OTHER; note = 2'-O-methoxyethyl guanosine 3'-phosphorothioate
  modified_base 3: mod_base = OTHER; note = 2'-O-methyl guanosine 3'-phosphodiester
  modified_base 4: mod_base = OTHER; note = 2'-O-methyl adenosine 3'-phosphodiester
  modified_base 5: mod_base = OTHER; note = 2'-O-methyl cytidine 3'-phosphodiester
  modified_base 6: mod_base = OTHER; note = 2'-O-methyl adenosine 3'-phosphodiester
  modified_base 7: mod_base = OTHER; note = 2'-O-methyl guanosine 3'-phosphodiester
  modified_base 8: mod_base = OTHER; note = 2'-O-methyl uridine 3'-phosphodiester
  modified_base 9: mod_base = OTHER; note = 2'-O-methyl adenosine 3'-phosphodiester
  modified_base 10: mod_base = OTHER; note = 2'-fluoro uridine 3'-phosphorothioate
  modified_base 11: mod_base = OTHER; note = 2'-fluoro uridine 3'-phosphodiester
  modified_base 12: mod_base = OTHER; note = 2'-O-methyl cytidine 3'-phosphodiester
  modified_base 13: mod_base = OTHER; note = 2'-O-methyl uridine 3'-phosphodiester
  modified_base 14: mod_base = OTHER; note = 2'-O-methyl cytidine 3'-phosphodiester
  modified_base 15: mod_base = OTHER; note = 2'-O-methyl adenosine 3'-phosphodiester
  modified_base 16: mod_base = OTHER; note = 2'-O-methyl guanosine 3'-phosphodiester
  modified_base 17: mod_base = OTHER; note = 2'-O-methyl uridine 3'-phosphorothioate
  modified_base 18: mod_base = OTHER; note = 2'-O-methoxyethyl guanosine 3'-phosphorothioate
  modified_base 19: mod_base = OTHER; note = 2'-O-methoxyethyl adenosine
gggacagtattctcagtga

SEQ ID NO: 87 - RNA, 19 nt, synthetic construct
  source 1..19: mol_type = other RNA
  modified_base 1..2: mod_base = OTHER; note = 2'-O-methoxyethyl guanosine 3'-phosphorothioate
  modified_base 3: mod_base = OTHER; note = 2'-O-methyl guanosine 3'-phosphodiester
  modified_base 4: mod_base = OTHER; note = 2'-O-methyl adenosine 3'-phosphodiester
  modified_base 5: mod_base = OTHER; note = 2'-O-methyl cytidine 3'-phosphodiester
  modified_base 6: mod_base = OTHER; note = 2'-O-methyl adenosine 3'-phosphodiester
  modified_base 7: mod_base = OTHER; note = 2'-O-methyl guanosine 3'-phosphodiester
  modified_base 8: mod_base = OTHER; note = 2'-O-methyl uridine 3'-phosphodiester
  modified_base 9: mod_base = OTHER; note = 2'-fluoro adenosine 3'-phosphorothioate
  modified_base 10: mod_base = OTHER; note = 2'-fluoro uridine 3'-phosphodiester
  modified_base 11: mod_base = OTHER; note = 2'-O-methyl uridine 3'-phosphodiester
  modified_base 12: mod_base = OTHER; note = 2'-O-methyl cytidine 3'-phosphodiester
  modified_base 13: mod_base = OTHER; note = 2'-O-methyl uridine 3'-phosphodiester
  modified_base 14: mod_base = OTHER; note = 2'-O-methyl cytidine 3'-phosphodiester
  modified_base 15: mod_base = OTHER; note = 2'-O-methyl adenosine 3'-phosphodiester
  modified_base 16: mod_base = OTHER; note = 2'-O-methyl guanosine 3'-phosphodiester
  modified_base 17: mod_base = OTHER; note = 2'-O-methyl uridine 3'-phosphorothioate
  modified_base 18: mod_base = OTHER; note = 2'-O-methoxyethyl guanosine 3'-phosphorothioate
  modified_base 19: mod_base = OTHER; note = 2'-O-methoxyethyl adenosine
gggacagtattctcagtga

SEQ ID NO: 88 - RNA, 22 nt, synthetic construct
  source 1..22: mol_type = other RNA
  modified_base 1: mod_base = OTHER; note = 2'-deoxythymidine 3'-phosphodiester
  modified_base 2..3: mod_base = OTHER; note = 2'-O-methyl adenosine 3'-phosphorothioate
  modified_base 4..6: mod_base = OTHER; note = 2'-O-methyl guanosine 3'-phosphodiester
  modified_base 7: mod_base = OTHER; note = 2'-O-methyl adenosine 3'-phosphodiester
  modified_base 8: mod_base = OTHER; note = 2'-fluoro cytidine 3'-phosphodiester
  modified_base 9: mod_base = OTHER; note = 2'-O-methyl adenosine 3'-phosphodiester
  modified_base 10: mod_base = OTHER; note = 2'-fluoro guanosine 3'-phosphodiester
  modified_base 11: mod_base = OTHER; note = 2'-fluoro uridine 3'-phosphodiester
  modified_base 12: mod_base = OTHER; note = 2'-fluoro adenosine 3'-phosphodiester
  modified_base 13..14: mod_base = OTHER; note = 2'-O-methyl uridine 3'-phosphodiester
  modified_base 15: mod_base = OTHER; note = 2'-O-methyl cytidine 3'-phosphodiester
  modified_base 16: mod_base = OTHER; note = 2'-O-methyl uridine 3'-phosphodiester
  modified_base 17: mod_base = OTHER; note = 2'-O-methyl cytidine 3'-phosphodiester
  modified_base 18: mod_base = OTHER; note = 2'-O-methyl adenosine 3'-phosphodiester
  modified_base 19: mod_base = OTHER; note = 2'-O-methyl guanosine 3'-phosphodiester
  modified_base 20: mod_base = OTHER; note = 2'-O-methyl uridine 3'-phosphorothioate
  modified_base 21: mod_base = OTHER; note = 2'-O-methyl guanosine 3'-phosphorothioate
  modified_base 22: mod_base = OTHER; note = 2'-O-methyl adenosine
taagggacagtattctcagtga

SEQ ID NO: 89 - RNA, 21 nt, synthetic construct
  source 1..21: mol_type = other RNA
  modified_base 1..2: mod_base = OTHER; note = 2'-O-methyl adenosine 3'-phosphorothioate
  modified_base 3..5: mod_base = OTHER; note = 2'-O-methyl guanosine 3'-phosphodiester
  modified_base 6: mod_base = OTHER; note = 2'-O-methyl adenosine 3'-phosphodiester
  modified_base 7: mod_base = OTHER; note = 2'-fluoro cytidine 3'-phosphodiester
  modified_base 8: mod_base = OTHER; note = 2'-O-methyl adenosine 3'-phosphodiester
  modified_base 9: mod_base = OTHER; note = 2'-fluoro guanosine 3'-phosphodiester
  modified_base 10: mod_base = OTHER; note = 2'-fluoro uridine 3'-phosphodiester
  modified_base 11: mod_base = OTHER; note = 2'-fluoro adenosine 3'-phosphodiester
  modified_base 12..13: mod_base = OTHER; note = 2'-O-methyl uridine 3'-phosphodiester
  modified_base 14: mod_base = OTHER; note = 2'-O-methyl cytidine 3'-phosphodiester
  modified_base 15: mod_base = OTHER; note = 2'-O-methyl uridine 3'-phosphodiester
  modified_base 16: mod_base = OTHER; note = 2'-O-methyl cytidine 3'-phosphodiester
  modified_base 17: mod_base = OTHER; note = 2'-O-methyl adenosine 3'-phosphodiester
  modified_base 18: mod_base = OTHER; note = 2'-O-methyl guanosine 3'-phosphodiester
  modified_base 19: mod_base = OTHER; note = 2'-O-methyl uridine 3'-phosphorothioate
  modified_base 20: mod_base = OTHER; note = 2'-O-methyl guanosine 3'-phosphorothioate
  modified_base 21: mod_base = OTHER; note = 2'-O-methyl adenosine
aagggacagtattctcagtga

SEQ ID NO: 90 - DNA, 22 bp, synthetic construct
  source 1..22: mol_type = other DNA
tcagcttcatgcagggttacat

SEQ ID NO: 91 - DNA, 19 bp, synthetic construct
  source 1..19: mol_type = other DNA
acgctgctcagtgcatcct

SEQ ID NO: 92 - DNA, 21 bp, synthetic construct
  source 1..21: mol_type = other DNA
aagcacgccaccaagaccgcc

SEQ ID NO: 93 - DNA, 19 bp, synthetic construct
  source 1..19: mol_type = other DNA
gaaggtgaaggtcggagtc

SEQ ID NO: 94 - DNA, 20 bp, synthetic construct
  source 1..20: mol_type = other DNA
gaagatggtgatgggatttc

SEQ ID NO: 95 - DNA, 20 bp, synthetic construct
  source 1..20: mol_type = other DNA
caagcttcccgttctcagcc

SEQ ID NO: 96 - RNA, 23 nt, synthetic construct
  source 1..23: mol_type = other RNA
  modified_base 1: mod_base = OTHER; note = 2'-O-methoxyethyl thymidine 3'-mesyl phosphoramidate, conjugated at the 5' end to vinyl phosphonate
  modified_base 2: mod_base = OTHER; note = 2'-fluoro cytidine 3'-mesyl phosphoramidate
  modified_base 3: mod_base = OTHER; note = 2'-O-methyl adenosine 3'-phosphodiester
  modified_base 4: mod_base = OTHER; note = 2'-O-methyl cytidine 3'-phosphodiester
  modified_base 5: mod_base = OTHER; note = 2'-O-methyl uridine 3'-phosphodiester
  modified_base 6: mod_base = OTHER; note = 2'-fluoro guanosine 3'-phosphodiester
  modified_base 7: mod_base = OTHER; note = 2'-O-methyl adenosine 3'-phosphodiester
  modified_base 8: mod_base = OTHER; note = 2'-O-methyl guanosine 3'-phosphodiester
  modified_base 9..10: mod_base = OTHER; note = 2'-O-methyl adenosine 3'-phosphodiester
  modified_base 11: mod_base = OTHER; note = 2'-O-methyl uridine 3'-phosphodiester
  modified_base 12: mod_base = OTHER; note = 2'-O-methyl adenosine 3'-phosphodiester
  modified_base 13: mod_base = OTHER; note = 2'-O-methyl cytidine 3'-phosphodiester
  modified_base 14: mod_base = OTHER; note = 2'-fluoro uridine 3'-phosphodiester
  modified_base 15: mod_base = OTHER; note = 2'-O-methyl guanosine 3'-phosphodiester
  modified_base 16: mod_base = OTHER; note = 2'-fluoro uridine 3'-phosphodiester
  modified_base 17..19: mod_base = OTHER; note = 2'-O-methyl cytidine 3'-phosphodiester
  modified_base 20: mod_base = OTHER; note = 2'-O-methyl uridine 3'-phosphodiester
  modified_base 21: mod_base = OTHER; note = 2'-O-methyl uridine 3'-mesyl phosphoramidate
  modified_base 22: mod_base = OTHER; note = 2'-O-methyl uridine 3'-phosphorothioate
  modified_base 23: mod_base = OTHER; note = 2'-O-methyl uridine
tcactgagaatactgtccctttt

SEQ ID NO: 97 - RNA, 23 nt, synthetic construct
  source 1..23: mol_type = other RNA
  modified_base 1: mod_base = OTHER; note = 2'-O-methoxyethyl thymidine 3'-phosphorothioate, conjugated at the 5' end to vinyl phosphonate
  modified_base 2: mod_base = OTHER; note = 2'-fluoro cytidine 3'-phosphorothioate
  modified_base 3: mod_base = OTHER; note = 2'-O-methyl adenosine 3'-phosphodiester
  modified_base 4: mod_base = OTHER; note = 2'-O-methyl cytidine 3'-phosphodiester
  modified_base 5: mod_base = OTHER; note = 2'-O-methyl uridine 3'-phosphodiester
  modified_base 6: mod_base = OTHER; note = 2'-fluoro guanosine 3'-phosphodiester
  modified_base 7: mod_base = OTHER; note = 2'-O-methyl adenosine 3'-phosphodiester
  modified_base 8: mod_base = OTHER; note = 2'-O-methyl guanosine 3'-phosphodiester
  modified_base 9..10: mod_base = OTHER; note = 2'-O-methyl adenosine 3'-phosphodiester
  modified_base 11: mod_base = OTHER; note = 2'-O-methyl uridine 3'-phosphodiester
  modified_base 12: mod_base = OTHER; note = 2'-O-methyl adenosine 3'-phosphodiester
  modified_base 13: mod_base = OTHER; note = 2'-O-methyl cytidine 3'-phosphodiester
  modified_base 14: mod_base = OTHER; note = 3'-fluoro hexitol nucleic acid uridine 3'-phosphodiester
  modified_base 15: mod_base = OTHER; note = 2'-O-methyl guanosine 3'-phosphodiester
  modified_base 16: mod_base = OTHER; note = 2'-fluoro uridine 3'-phosphodiester
  modified_base 17..19: mod_base = OTHER; note = 2'-O-methyl cytidine 3'-phosphodiester
  modified_base 20: mod_base = OTHER; note = 2'-O-methyl uridine 3'-phosphodiester
  modified_base 21..22: mod_base = OTHER; note = 2'-O-methyl uridine 3'-phosphorothioate
  modified_base 23: mod_base = OTHER; note = 2'-O-methyl uridine
tcactgagaatactgtccctttt

SEQ ID NO: 98 - RNA, 23 nt, synthetic construct
  source 1..23: mol_type = other RNA
  modified_base 1: mod_base = OTHER; note = 2'-O-methoxyethyl thymidine 3'-phosphorothioate, conjugated at the 5' end to vinyl phosphonate
  modified_base 2: mod_base = OTHER; note = 2'-fluoro cytidine 3'-mesyl phosphoramidate
  modified_base 3: mod_base = OTHER; note = 2'-O-methyl adenosine 3'-phosphodiester
  modified_base 4: mod_base = OTHER; note = 2'-O-methyl cytidine 3'-phosphodiester
  modified_base 5: mod_base = OTHER; note = 2'-O-methyl uridine 3'-phosphodiester
  modified_base 6: mod_base = OTHER; note = 2'-deoxyguanosine 3'-phosphorothioate
  modified_base 7: mod_base = OTHER; note = 2'-O-methyl adenosine 3'-phosphodiester
  modified_base 8: mod_base = OTHER; note = 2'-O-methyl guanosine 3'-phosphodiester
  modified_base 9..10: mod_base = OTHER; note = 2'-O-methyl adenosine 3'-phosphodiester
  modified_base 11: mod_base = OTHER; note = 2'-O-methyl uridine 3'-phosphodiester
  modified_base 12: mod_base = OTHER; note = 2'-O-methyl adenosine 3'-phosphodiester
  modified_base 13: mod_base = OTHER; note = 2'-O-methyl cytidine 3'-phosphodiester
  modified_base 14: mod_base = OTHER; note = 2'-fluoro uridine 3'-phosphodiester
  modified_base 15: mod_base = OTHER; note = 2'-O-methyl guanosine 3'-phosphodiester
  modified_base 16: mod_base = OTHER; note = 2'-fluoro uridine 3'-phosphodiester
  modified_base 17..19: mod_base = OTHER; note = 2'-O-methyl cytidine 3'-phosphodiester
  modified_base 20: mod_base = OTHER; note = 2'-O-methyl uridine 3'-phosphodiester
  modified_base 21..22: mod_base = OTHER; note = 2'-O-methyl uridine 3'-phosphorothioate
  modified_base 23: mod_base = OTHER; note = 2'-O-methyl uridine
tcactgagaatactgtccctttt

SEQ ID NO: 99 - RNA, 23 nt, synthetic construct
  source 1..23: mol_type = other RNA
  modified_base 1: mod_base = OTHER; note = 2'-O-methoxyethyl thymidine 3'-phosphorothioate, conjugated at the 5' end to vinyl phosphonate
  modified_base 2: mod_base = OTHER; note = 2'-fluoro cytidine 3'-mesyl phosphoramidate
  modified_base 3: mod_base = OTHER; note = 2'-O-methyl adenosine 3'-phosphodiester
  modified_base 4: mod_base = OTHER; note = 2'-O-methyl cytidine 3'-phosphodiester
  modified_base 5: mod_base = OTHER; note = 2'-O-methyl uridine 3'-phosphodiester
  modified_base 6: mod_base = OTHER; note = 2'-deoxyguanosine 3'-phosphorothioate
  modified_base 7: mod_base = OTHER; note = 2'-O-methyl adenosine 3'-phosphodiester
  modified_base 8: mod_base = OTHER; note = 2'-O-methyl guanosine 3'-phosphodiester
  modified_base 9..10: mod_base = OTHER; note = 2'-O-methyl adenosine 3'-phosphodiester
  modified_base 11: mod_base = OTHER; note = 2'-O-methyl uridine 3'-phosphodiester
  modified_base 12: mod_base = OTHER; note = 2'-O-methyl adenosine 3'-phosphodiester
  modified_base 13: mod_base = OTHER; note = 2'-O-methyl cytidine 3'-phosphodiester
  modified_base 14: mod_base = OTHER; note = 2'-fluoro uridine 3'-mesyl phosphoramidate
  modified_base 15: mod_base = OTHER; note = 2'-O-methyl guanosine 3'-phosphodiester
  modified_base 16: mod_base = OTHER; note = 2'-fluoro uridine 3'-mesyl phosphoramidate
  modified_base 17..19: mod_base = OTHER; note = 2'-O-methyl cytidine 3'-phosphodiester
  modified_base 20: mod_base = OTHER; note = 2'-O-methyl uridine 3'-phosphodiester
  modified_base 21..22: mod_base = OTHER; note = 2'-O-methyl uridine 3'-phosphorothioate
  modified_base 23: mod_base = OTHER; note = 2'-O-methyl uridine
tcactgagaatactgtccctttt

SEQ ID NO: 100 - RNA, 23 nt, synthetic construct
  source 1..23: mol_type = other RNA
  modified_base 1: mod_base = OTHER; note = 2'-O-methoxyethyl thymidine 3'-phosphorothioate, conjugated at the 5' end to vinyl phosphonate
  modified_base 2: mod_base = OTHER; note = 2'-fluoro cytidine 3'-phosphorothioate
  modified_base 3: mod_base = OTHER; note = 2'-O-methyl adenosine 3'-phosphodiester
  modified_base 4: mod_base = OTHER; note = 2'-O-methyl cytidine 3'-phosphodiester
  modified_base 5: mod_base = OTHER; note = 2'-O-methyl uridine 3'-phosphodiester
  modified_base 6: mod_base = OTHER; note = 2'-O-methyl guanosine 3'-phosphodiester
  modified_base 7: mod_base = OTHER; note = 2'-O-methyl adenosine 3'-phosphodiester
  modified_base 8: mod_base = OTHER; note = 2'-O-methyl guanosine 3'-phosphodiester
  modified_base 9..10: mod_base = OTHER; note = 2'-O-methyl adenosine 3'-phosphodiester
  modified_base 11: mod_base = OTHER; note = 2'-O-methyl uridine 3'-phosphodiester
  modified_base 12: mod_base = OTHER; note = 2'-O-methyl adenosine 3'-phosphodiester
  modified_base 13: mod_base = OTHER; note = 2'-O-methyl cytidine 3'-phosphodiester
  modified_base 14: mod_base = OTHER; note = 2'-fluoro uridine 3'-phosphodiester
  modified_base 15: mod_base = OTHER; note = 2'-O-methyl guanosine 3'-phosphodiester
  modified_base 16: mod_base = OTHER; note = 2'-O-methyl uridine 3'-phosphodiester
  modified_base 17..19: mod_base = OTHER; note = 2'-O-methyl cytidine 3'-phosphodiester
  modified_base 20: mod_base = OTHER; note = 2'-O-methyl uridine 3'-phosphodiester
  modified_base 21: mod_base = OTHER; note = 2'-O-methyl uridine 3'-phosphorothioate
  modified_base 22: mod_base = OTHER; note = 2'-O-methoxyethyl thymidine 3'-phosphorothioate
  modified_base 23: mod_base = OTHER; note = 2'-O-methoxyethyl thymidine
tcactgagaatactgtccctttt

SEQ ID NO: 101 - RNA, 23 nt, synthetic construct
  source 1..23: mol_type = other RNA
  modified_base 1: mod_base = OTHER; note = 2'-O-methoxyethyl thymidine 3'-phosphorothioate, conjugated at the 5' end to vinyl phosphonate
  modified_base 2: mod_base = OTHER; note = 2'-fluoro cytidine 3'-phosphorothioate
  modified_base 3: mod_base = OTHER; note = 2'-O-methyl adenosine 3'-phosphodiester
  modified_base 4: mod_base = OTHER; note = 2'-O-methyl cytidine 3'-phosphodiester
  modified_base 5: mod_base = OTHER; note = 2'-O-methyl uridine 3'-phosphodiester
  modified_base 6: mod_base = OTHER; note = 2'-O-methyl guanosine 3'-phosphodiester
  modified_base 7: mod_base = OTHER; note = 2'-O-methyl adenosine 3'-phosphodiester
  modified_base 8: mod_base = OTHER; note = 2'-O-methyl guanosine 3'-phosphodiester
  modified_base 9..10: mod_base = OTHER; note = 2'-O-methyl adenosine 3'-phosphodiester
  modified_base 11: mod_base = OTHER; note = 2'-O-methyl uridine 3'-phosphodiester
  modified_base 12: mod_base = OTHER; note = 2'-O-methyl adenosine 3'-phosphodiester
  modified_base 13: mod_base = OTHER; note = 2'-O-methyl cytidine 3'-phosphodiester
  modified_base 14: mod_base = OTHER; note = 2'-fluoro uridine 3'-phosphodiester
  modified_base 15: mod_base = OTHER; note = 2'-O-methyl guanosine 3'-phosphodiester
  modified_base 16: mod_base = OTHER; note = 2'-O-methyl uridine 3'-phosphodiester
  modified_base 17..19: mod_base = OTHER; note = 2'-O-methyl cytidine 3'-phosphodiester
  modified_base 20: mod_base = OTHER; note = 2'-O-methyl uridine 3'-phosphodiester
  modified_base 21: mod_base = OTHER; note = 2'-O-methyl uridine 3'-phosphorothioate
  modified_base 22: mod_base = OTHER; note = constrained ethyl thymidine 3'-phosphorothioate
  modified_base 23: mod_base = OTHER; note = constrained ethyl thymidine
tcactgagaatactgtccctttt

SEQ ID NO: 102 - RNA, 23 nt, synthetic construct
  source 1..23: mol_type = other RNA
  modified_base 1: mod_base = OTHER; note = 2'-O-methoxyethyl thymidine 3'-phosphorothioate, conjugated at the 5' end to vinyl phosphonate
  modified_base 2: mod_base = OTHER; note = 2'-fluoro cytidine 3'-phosphorothioate
  modified_base 3: mod_base = OTHER; note = 2'-O-methyl adenosine 3'-phosphodiester
  modified_base 4: mod_base = OTHER; note = 2'-O-methyl cytidine 3'-phosphodiester
  modified_base 5: mod_base = OTHER; note = 2'-O-methyl uridine 3'-phosphodiester
  modified_base 6: mod_base = OTHER; note = 2'-deoxyguanosine 3'-phosphorothioate
  modified_base 7: mod_base = OTHER; note = 2'-O-methyl adenosine 3'-phosphodiester
  modified_base 8: mod_base = OTHER; note = 2'-O-methyl guanosine 3'-phosphodiester
  modified_base 9..10: mod_base = OTHER; note = 2'-O-methyl adenosine 3'-phosphodiester
  modified_base 11: mod_base = OTHER; note = 2'-O-methyl uridine 3'-phosphodiester
  modified_base 12: mod_base = OTHER; note = 2'-O-methyl adenosine 3'-phosphodiester
  modified_base 13: mod_base = OTHER; note = 2'-O-methyl cytidine 3'-phosphodiester
  modified_base 14: mod_base = OTHER; note = 2'-fluoro uridine 3'-phosphodiester
  modified_base 15: mod_base = OTHER; note = 2'-O-methyl guanosine 3'-phosphodiester
  modified_base 16: mod_base = OTHER; note = 2'-fluoro uridine 3'-phosphodiester
  modified_base 17..19: mod_base = OTHER; note = 2'-O-methyl cytidine 3'-phosphodiester
  modified_base 20: mod_base = OTHER; note = 2'-deoxythymidine 3'-phosphorothioate
  modified_base 21: mod_base = OTHER; note = constrained ethyl thymidine 3'-phosphorothioate
  modified_base 22: mod_base = OTHER; note = 2'-O-methoxyethyl adenosine 3'-phosphorothioate
  modified_base 23: mod_base = OTHER; note = 2'-O-methoxyethyl adenosine
tcactgagaatactgtcccttaa

SEQ ID NO: 103 - DNA, 23 bp, synthetic construct
  source 1..23: mol_type = other DNA
  modified_base 1: mod_base = OTHER; note = thymine
  modified_base 20..21: mod_base = OTHER; note = thymine
tcactgagaatactgtcccttaa

SEQ ID NO: 104 - RNA, 21 nt, synthetic construct
  source 1..21: mol_type = other RNA
  modified_base 1: mod_base = OTHER; note = 2'-O-methyl adenosine 3'-phosphorothioate, conjugated at the 5' end to trishexylamino-N-Acetylgalactosamine-6-aminohexyl phosphodiester conjugate
  modified_base 2: mod_base = OTHER; note = 2'-O-methyl adenosine 3'-phosphorothioate
  modified_base 3..5: mod_base = OTHER; note = 2'-O-methyl guanosine 3'-phosphodiester
  modified_base 6: mod_base = OTHER; note = 2'-O-methyl adenosine 3'-phosphodiester
  modified_base 7: mod_base = OTHER; note = 3'-fluoro hexitol nucleic acid cytidine 3'-phosphodiester
  modified_base 8: mod_base = OTHER; note = 2'-O-methyl adenosine 3'-phosphodiester
  modified_base 9: mod_base = OTHER; note = 2'-fluoro guanosine 3'-phosphodiester
  modified_base 10: mod_base = OTHER; note = 2'-fluoro uridine 3'-phosphodiester
  modified_base 11: mod_base = OTHER; note = 3'-fluoro hexitol nucleic acid adenosine 3'-phosphodiester
  modified_base 12..13: mod_base = OTHER; note = 2'-O-methyl uridine 3'-phosphodiester
  modified_base 14: mod_base = OTHER; note = 2'-O-methyl cytidine 3'-phosphodiester
  modified_base 15: mod_base = OTHER; note = 2'-O-methyl uridine 3'-phosphodiester
  modified_base 16: mod_base = OTHER; note = 2'-O-methyl cytidine 3'-phosphodiester
  modified_base 17: mod_base = OTHER; note = 2'-O-methyl adenosine 3'-phosphodiester
  modified_base 18: mod_base = OTHER; note = 2'-O-methyl guanosine 3'-phosphodiester
  modified_base 19: mod_base = OTHER; note = 2'-O-methyl uridine 3'-phosphorothioate
  modified_base 20: mod_base = OTHER; note = 2'-O-methyl guanosine 3'-phosphorothioate
  modified_base 21: mod_base = OTHER; note = 2'-O-methyl adenosine
aagggacagtattctcagtga

SEQ ID NO: 105 - RNA, 21 nt, synthetic construct
  source 1..21: mol_type = other RNA
  modified_base 1: mod_base = OTHER; note = 2'-O-methyl adenosine 3'-phosphorothioate, conjugated at the 5' end to trishexylamino-N-Acetylgalactosamine-6-aminohexyl phosphodiester conjugate
  modified_base 2: mod_base = OTHER; note = 2'-O-methyl adenosine 3'-phosphorothioate
  modified_base 3..5: mod_base = OTHER; note = 2'-O-methyl guanosine 3'-phosphodiester
  modified_base 6: mod_base = OTHER; note = 2'-O-methyl adenosine 3'-phosphodiester
  modified_base 7: mod_base = OTHER; note = 2'-O-methyl cytidine 3'-phosphodiester
  modified_base 8: mod_base = OTHER; note = 2'-O-methyl adenosine 3'-phosphodiester
  modified_base 9: mod_base = OTHER; note = constrained ethyl guanosine 3'-phosphodiester
  modified_base 10: mod_base = OTHER; note = 2'-deoxyuridine 3'-phosphodiester
  modified_base 11: mod_base = OTHER; note = constrained ethyl adenosine 3'-phosphodiester
  modified_base 12..13: mod_base = OTHER; note = 2'-O-methyl uridine 3'-phosphodiester
  modified_base 14: mod_base = OTHER; note = 2'-O-methyl cytidine 3'-phosphodiester
  modified_base 15: mod_base = OTHER; note = 2'-O-methyl uridine 3'-phosphodiester
  modified_base 16: mod_base = OTHER; note = 2'-O-methyl cytidine 3'-phosphodiester
  modified_base 17: mod_base = OTHER; note = 2'-O-methyl adenosine 3'-phosphodiester
  modified_base 18: mod_base = OTHER; note = 2'-O-methyl guanosine 3'-phosphodiester
  modified_base 19: mod_base = OTHER; note = 2'-O-methyl uridine 3'-phosphorothioate
  modified_base 20: mod_base = OTHER; note = 2'-O-methyl guanosine 3'-phosphorothioate
  modified_base 21: mod_base = OTHER; note = 2'-O-methyl adenosine
aagggacagtattctcagtga

SEQ ID NO: 106 - RNA, 21 nt, synthetic construct
  source 1..21: mol_type = other RNA
  modified_base 1: mod_base = OTHER; note = 2'-O-methyl adenosine 3'-phosphorothioate, conjugated at the 5' end to trishexylamino-N-Acetylgalactosamine-6-aminohexyl phosphodiester conjugate
  modified_base 2: mod_base = OTHER; note = 2'-O-methyl adenosine 3'-phosphorothioate
  modified_base 3..5: mod_base = OTHER; note = 2'-O-methyl guanosine 3'-phosphodiester
  modified_base 6: mod_base = OTHER; note = 2'-O-methyl adenosine 3'-phosphodiester
  modified_base 7: mod_base = OTHER; note = 2'-O-methyl cytidine 3'-phosphodiester
  modified_base 8: mod_base = OTHER; note = 2'-O-methyl adenosine 3'-phosphodiester
  modified_base 9: mod_base = OTHER; note = 2'-O-methyl guanosine 3'-phosphodiester
  modified_base 10: mod_base = OTHER; note = 2'-O-methyl uridine 3'-phosphodiester
  modified_base 11: mod_base = OTHER; note = 2'-O-methyl adenosine 3'-phosphodiester
  modified_base 12..13: mod_base = OTHER; note = 2'-O-methyl uridine 3'-phosphodiester
  modified_base 14: mod_base = OTHER; note = 2'-O-methyl cytidine 3'-phosphodiester
  modified_base 15: mod_base = OTHER; note = 2'-O-methyl uridine 3'-phosphodiester
  modified_base 16: mod_base = OTHER; note = 2'-O-methyl cytidine 3'-phosphodiester
  modified_base 17: mod_base = OTHER; note = 2'-O-methyl adenosine 3'-phosphodiester
  modified_base 18: mod_base = OTHER; note = 2'-O-methyl guanosine 3'-phosphodiester
  modified_base 19: mod_base = OTHER; note = 2'-O-methyl uridine 3'-phosphorothioate
  modified_base 20: mod_base = OTHER; note = 2'-O-methyl guanosine 3'-phosphorothioate
  modified_base 21: mod_base = OTHER; note = 2'-O-methyl adenosine
aagggacagtattctcagtga

SEQ ID NO: 107 - DNA, 16 bp, synthetic construct
  source 1..16: mol_type = other DNA
cgacggcgagcctttg

SEQ ID NO: 108 - DNA, 23 bp, synthetic construct
  source 1..23: mol_type = other DNA
tctgctgtctttggaaccttgtc

SEQ ID NO: 109 - DNA, 23 bp, synthetic construct
  source 1..23: mol_type = other DNA
cgcgtctccttcgagctgtttgc

SEQ ID NO: 110 - DNA, 23 bp, synthetic construct
  source 1..23: mol_type = other DNA
  modified_base 1: mod_base = OTHER; note = thymine
  modified_base 22..23: mod_base = OTHER; note = thymine
tcactgagaatactgtccctttt

SEQ ID NO: 111 - RNA, 21 nt, synthetic construct
  source 1..21: mol_type = other RNA
  modified_base 1: mod_base = OTHER; note = 2'-O-methyl adenosine 3'-phosphorothioate, conjugated at the 5' end to trishexylamino-N-Acetylgalactosamine-6-aminohexyl phosphodiester conjugate
  modified_base 2: mod_base = OTHER; note = 2'-O-methyl adenosine 3'-phosphorothioate
  modified_base 3..5: mod_base = OTHER; note = 2'-O-methyl guanosine 3'-phosphodiester
  modified_base 6: mod_base = OTHER; note = 2'-O-methyl adenosine 3'-phosphodiester
  modified_base 7: mod_base = OTHER; note = 2'-O-methyl cytidine 3'-phosphodiester
  modified_base 8: mod_base = OTHER; note = 2'-O-methyl adenosine 3'-phosphodiester
  modified_base 9: mod_base = OTHER; note = 2'-O-methyl guanosine 3'-phosphodiester
  modified_base 10: mod_base = OTHER; note = 2'-fluoro uridine 3'-phosphodiester
  modified_base 11: mod_base = OTHER; note = 2'-fluoro adenosine 3'-phosphodiester
  modified_base 12..13: mod_base = OTHER; note = 2'-O-methyl uridine 3'-phosphodiester
  modified_base 14: mod_base = OTHER; note = 2'-O-methyl cytidine 3'-phosphodiester
  modified_base 15: mod_base = OTHER; note = 2'-O-methyl uridine 3'-phosphodiester
  modified_base 16: mod_base = OTHER; note = 2'-O-methyl cytidine 3'-phosphodiester
  modified_base 17: mod_base = OTHER; note = 2'-O-methyl adenosine 3'-phosphodiester
  modified_base 18: mod_base = OTHER; note = 2'-O-methyl guanosine 3'-phosphodiester
  modified_base 19: mod_base = OTHER; note = 2'-O-methyl uridine 3'-phosphorothioate
  modified_base 20: mod_base = OTHER; note = 2'-O-methyl guanosine 3'-phosphorothioate
  modified_base 21: mod_base = OTHER; note = 2'-O-methyl adenosine
aagggacagtattctcagtga

(19) United States
(12) Patent Application Publication
(10) Pub. No.: US 2025/0243487 A1
(43) Pub. Date: Jul. 31, 2025

(54) Compounds and Methods for Reducing APOCIII Expression

(71) Applicant: Ionis Pharmaceuticals, Inc., Carlsbad, CA (US)

(72) Inventors: Adam Mullick, Carlsbad, CA (US); Thazha P. Prakash, Carlsbad, CA (US); Eric E. Swayze, Encinitas, CA (US)

(73) Assignee: Ionis Pharmaceuticals, Inc., Carlsbad, CA (US)

(21) Appl. No.: 18/884,746

(22) Filed: Sep. 13, 2024

Related U.S. Application Data
(60) Provisional application No. 63/582,688, filed on Sep. 14, 2023.
(60) Provisional application No. 63/648,076, filed on May 15, 2024.

(51) Int. Cl. C12N 15/113; A61P 9/10

(52) U.S. Cl. CPC C12N 15/113; A61P 9/10; C12N 2310/3125; C12N 2310/315; C12N 2310/321; C12N 2310/322; C12N 2310/351; C12N 2320/32

(57) ABSTRACT

Provided are oligomeric agents, methods, and pharmaceutical compositions for reducing the amount or activity of APOC3 RNA in a cell or animal, and in certain instances reducing the amount of ApoCIII protein in a cell or animal. Such oligomeric agents, methods, and pharmaceutical compositions are useful to treat or manage hypertriglyceridemia and/or cardiovascular disease (CVD).

### CROSS-REFERENCE TO RELATED APPLICATIONS

This application claims the benefit of priority to U.S. Provisional Application No. 63/582,688, filed Sep. 14, 2023, and U.S. Provisional Application No. 63/648,076, filed May 15, 2024, each of which is incorporated by reference herein in its entirety for any purpose.

### SEQUENCE LISTING

The present application is being filed concurrently with a Sequence Listing in electronic format. The Sequence Listing is provided as a file entitled BIOL0470SEQ.xml, created on Aug. 29, 2024, which is 1,090 KB in size. The contents of the electronic format of the sequence listing are incorporated herein by reference in their entirety.

### FIELD

The present invention relates to agents, compositions, and uses therefor, including methods for decreasing the levels of APOC3 expression, APOC3 RNA, and/or the levels (and/or activity) of ApoCIII protein, as well as to methods for preventing, treating, and/or ameliorating at least one symptom of an ApoCIII related disease or disorder, including, e.g., hypertriglyceridemia, dyslipidemia, pancreatitis, familial chylomicronemia syndrome (FCS), severe hypertriglyceridemia (sHTG), atherosclerotic cardiovascular disease (ASCVD), other cardiometabolic disorders.

### BACKGROUND

The incidence of severe hypertriglyceridemia (sHTG) is rising globally, and according to GlobalData epidemiologists, the US alone will reach over 17 million prevalent cases by 2025. s-ITG often occurs secondary to familial chylomicronemia syndrome (FCS), but also results from environmental factors including high-fat diets and sedentary lifestyles. In sHTG, triglyceride levels are greatly elevated (>500 mg/dL), increasing the risk of atherosclerotic cardiovascular disease (ASCVD), pancreatitis, and death.

Apolipoprotein C-III (also called APOC3, APOC-III, ApoCIII, and APO C-III) is a constituent of HDL and of triglyceride (TG)-rich lipoproteins; and elevated ApoCIII levels are associated with elevated TG levels and diseases such as cardiovascular disease, metabolic syndrome, obesity and diabetes (Chan et al., Int J Clin Pract, 2008, 62:799-809; Onat et at., Atherosclerosis, 2003, 168:81-89; Mendivil et al., Circulation, 2011, 124:2065-2072; Mauger et al., J. Lipid Res, 2006. 47: 1212-1218; Chan et al., Clin. Chem, 2002. 278-283; Ooi et al., Clin. Sci, 2008. 114: 611-624; Davidsson et al., J. Lipid Res. 2005. 46: 1999-2006; Sacks et al., Circulation, 2000. 102: 1886-1892; Lee et al., Arterioscler Thromb Vasc Biol, 2003. 23: 853-858). ApoCIII slows clearance of TG-rich lipoproteins by inhibiting breakdown of triglycerides and clearance of triglyceride-rich lipoproteins through inhibition of lipoprotein lipase (LPL) and interfering with lipoprotein binding to cell-surface glycosaminoglycan matrix (Shachter, Curr. Opin. Lipidol, 2001, 12, 297-304). Therapeutic agents are in clinical development for reducing expression of hepatic APOC3, in an effort to reduce ApoCIII protein activity, and thus lower triglyceride levels in patients at high risk for, or with established cardiovascular disease. For example, olezarsen is an antisense RNA oligonucleotide that inhibits the hepatic production of ApoCIII, thus enhancing clearance and decreasing triglyceride serum levels, and decreasing atherosclerotic risk. However, no agent targeting APOC3 have been approved for commercial use; accordingly, a need exists to provide patients with additional potent treatment options.

### SUMMARY

Provided herein are oligomeric duplexes, pharmaceutical compositions, and methods of use for reducing the amount or activity of APOC3 RNA and reducing the expression of ApoCIII protein in a cell or subject. In certain embodiments, the subject has a disease or disorder associated with triglyceride regulation, regulation of lipoproteins or a mutation in lipoprotein regulation pathway. In certain embodiments, the subject has a hypertriglyceridemia. In certain embodiments the subject has severe hypertriglyceridemia. In certain embodiments, the subject has FCS. In certain embodiments, agents useful for reducing the amount or activity of APOC3 RNA are oligomeric agents, oligomeric duplexes, antisense agents, RNAi agents. In certain embodiments, agents useful for decreasing expression of APOC3 are oligomeric agents, oligomeric duplexes, antisense agents, and/or RNAi agents.

Provided are modified oligonucleotides and agents and compositions comprising them, including, but not limited to, antisense agents, oligomeric agents, oligomeric duplexes and pharmaceutical compositions comprising modified oligonucleotides. In certain embodiments, a modified oligonucleotide provided herein comprises a nucleobase sequence at least 80% complementary to an equal length portion of a APOC3 nucleic acid. In certain embodiments, the modified oligonucleotide consists of 12 to 35, 14 to 30, 15 to 28, 16 to 25, or 18 to 23 linked nucleosides targeting APOC3 nucleic acid. In certain embodiments, a modified oligonucleotide provided herein comprises a sequence of nucleobases complementary to an equal length portion of the nucleobase sequence of SEQ ID NO: 1. In certain embodiments, provided oligomeric duplexes comprise a first oligomeric compound and a second oligomeric compound, wherein a first oligomeric compound comprises a modified oligonucleotide consisting of 18 to 50 linked nucleosides, wherein the nucleobase sequence of the first oligomeric compound comprises at least 18, at least 19, at least 20, at least 21, at least 22, or at least 23 contiguous nucleobases of the nucleobase sequence of any one of SEQ ID NOs: 2-5, wherein each of the nucleosides of the first oligomeric compound comprises a modified sugar moiety or sugar surrogate and wherein no more than 22%, no more than 20%, no more than 18%, no more than 15%, no more than 10%, or no more than 5% of the modified nucleosides in the first oligomeric compound comprises a 2′-fluoro sugar moiety; and wherein a second oligomeric compound comprises a modified oligonucleotide consisting of 16 to 50 contiguous linked nucleosides wherein the nucleobase sequence of the second oligomeric compound comprises at least 16, at least 17, at least 18, at least 19, at least 20, or at least 21 contiguous nucleobases of the nucleobase sequence of any one of SEQ ID NOs: 6-8, wherein each of the nucleosides of the second oligomeric compound comprises a modified sugar moiety or sugar surrogate and wherein no more than 25%, no more than 20%, no more than 18%, no more than 16%, no more than 14%, no more than 12%, or no more than 10%, of the modified nucleosides in the second oligomeric compound comprises a 2′-fluoro sugar moiety. In certain embodiments, a modified oligonucleotide provided herein comprises at least one modified sugar moiety and/or at least one modified internucleoside linkage. Modified oligonucleotides and compositions comprising them, including, but not limited to, oligomeric agents, oligomeric duplexes, antisense agents and pharmaceutical compositions, described herein are useful for reducing or inhibiting APOC3 expression in a cell, organ, tissue, system, organism or animal.

In certain embodiments provided are oligomeric compounds comprising a modified oligonucleotide having a sequence selected from any one of SEQ ID NO: 11-34, and 51-74. In certain embodiments provided are oligomeric duplexes comprising an oligomeric compound selected from any one of SEQ ID NO: 35-49. In certain embodiments provided are oligomeric agents comprising an oligomeric compound selected from any one of SEQ ID NO: 75-89. In certain embodiments provided are oligomeric duplexes comprising a first oligomeric compound selected from any one of SEQ ID NO: 11-34, and 51-74, and comprising a second oligomeric compound selected from any one of SEQ ID NO: 35-49. In certain embodiments provided are oligomeric duplexes comprising a first oligomeric compound selected from any one of SEQ ID NO: 11-34, and 51-74, and comprising a second oligomeric compound selected from any one of SEQ ID NO: 75-89.

Additionally provided are methods for reducing or inhibiting APOC3 expression, APOC3 RNA levels and/or ApoCIII protein levels and/or activity in a cell or organism, including, for example, an animal. In certain embodiments, the methods include contacting a cell or subject, with a composition provided herein, comprising a modified oligonucleotide, oligomeric compound, and/or oligomeric duplex. In certain embodiments, the subject is a human who has or is at risk of having a disease, disorder, condition or injury associated with lipoprotein metabolism misregulation, misregulation of triglyceride turnover or a mutation in APOC3. In certain embodiments, the subject is a human who has or is at risk of having hypertriglyceridemia. In certain embodiments, the subject is a human who has or is at risk of having severe hypertriglyceridemia.

Provided herein are methods of treating a disease, disorder, condition or injury associated with lipoprotein metabolism misregulation, regulation of triglyceride turnover or a mutation in APOC3. In certain embodiment, a method of treating a disease, disorder, condition or injury associated with lipoprotein metabolism misregulation, regulation of triglyceride turnover or a mutation in APOC3 comprises administering to a subject, e.g., a human subject, having, or at risk of having, a disease, disorder or condition associated with lipoprotein metabolism misregulation, a provided oligomeric duplex, oligomeric compound, or composition provided herein, wherein the disease, disorder, condition or injury is selected from a cardiovascular disease, disorder, condition, a metabolic disease, disorder, or condition, or an inflammatory disease disorder or condition. In certain embodiments, methods of treating provided herein result in ameliorating (whether by reduced frequency, severity) a at least one symptom of a disease, disorder, condition or injury associated with lipoprotein metabolism misregulation. In certain embodiments, methods of treating provided herein result in preventing, delay or postponing, or slowing the development or progression of at least one symptom of a disease, disorder or condition associated with elevated triglycerides.

Also provided are methods useful for ameliorating at least one symptom of a disorder associated with lipoprotein metabolism misregulation. In certain embodiments the disorder is severe hypertriglyceridemia. In certain embodiments, the disorder is FCS. In certain embodiments the disorder is lipidemia. In certain embodiments, a symptom of hypertriglyceridemia and/or lipodystrophy include, but are not limited to, episodes of abdominal pain, physical fatigue, difficulty thinking, diarrhea, recurrent acute pancreatitis, eruptive cutaneous xanthomata, and hepatosplenomegaly or a combination thereof. In certain embodiments, methods provided herein for preventing, treating, ameliorating, delaying the onset of, or reducing frequency of at least one symptom of hypertriglyceridemia, include administering to a subject, e.g., a human subject, having or at risk of having at least one symptom a composition provided herein, e.g., a modified oligonucleotide, oligomeric compound, oligomeric duplex or pharmaceutical composition provided herein.

### DETAILED DESCRIPTION

It is to be understood that both the foregoing general description and the following detailed description are exemplary and explanatory only and are not restrictive. Herein, the use of the singular includes the plural unless specifically stated otherwise. Furthermore, the use of the term “including” as well as other forms, such as “includes” and “included”, is not limiting. Also, terms such as “element” or “component” encompass both elements and components comprising one unit and elements and components that comprise more than one subunit, unless specifically stated otherwise.

Section headings used herein are for organizational purposes only and are not to be construed as limiting the subject matter described. All documents, or portions of documents, cited in this application, including, but not limited to, patents, patent applications, articles, books, and treatises, are hereby expressly incorporated-by-reference for the portions of the document discussed herein, as well as in their entirety.

### Definitions

The following definitions are provided, along with additional definitions throughout the specification, for a complete understanding of the instant invention. Unless specific definitions are provided herein, nomenclature used in connection with, and procedures and techniques of, analytical chemistry, synthetic organic chemistry, and medicinal and pharmaceutical chemistry described herein are those well-known and commonly used in the art. Unless otherwise indicated, certain terms have the following meanings: As used herein, “2′-deoxynucleoside” means a nucleoside comprising a 2′-deoxy sugar moiety.

Unless otherwise indicated, a 2′-deoxynucleoside is a 2′-β-D-deoxynucleoside which comprises a 2′-β-D-deoxyribosyl sugar moiety, which has the β-D configuration in naturally occurring deoxyribonucleic acid (DNA). A 2′-deoxynucleoside or a nucleoside comprising an unmodified 2′-deoxyribosyl sugar moiety may be abasic, comprise a modified nucleobase, or may comprise an RNA nucleobase (uracil).

As used herein, “2′-deoxy sugar moiety” means a 2′-H(H) deoxyribosyl sugar moiety. Unless otherwise indicated, a 2′-deoxy sugar moiety is a 2′-β-D-deoxyribosyl sugar moiety, which has the β-D configuration in naturally occurring deoxyribonucleic acids (DNA). Herein, in the context of an oligomeric duplex comprising a ribonucleic acid (e.g., an siRNA) oligonucleotide, a 2′-deoxy sugar moiety is considered e.g., a modified sugar moiety.

As used herein, “2′-MOE” means a 2′-OCH₂CH₂OCH₃ group in place of the 2′-OH group of a ribosyl sugar moiety. A “2′-MOE sugar moiety” means a sugar moiety with a 2′-OCH₂CH₂OCH₃ group in place of the 2′-OH group of a ribosyl sugar moiety. Unless otherwise indicated, a 2′-MOE sugar moiety is in the β-D-ribosyl configuration. “MOE” means O-methoxyethyl.

As used herein, “2′-MOE nucleoside” or “2′-O(CH₂)₂OCH₃ nucleoside” means a nucleoside comprising a 2′-MOE sugar moiety (or 2′-OCH₂CH₂OCH₃ ribosyl sugar moiety).

As used herein, “2′-OMe” means a 2′-OCH₃ group in place of the 2′-OH group of a ribosyl sugar moiety. A “2′-OMe sugar moiety” means a sugar moiety with a 2′-OCH₃ group in place of the 2′-OH group of a ribosyl sugar moiety. Unless otherwise indicated, a 2′-OMe has the β-D-ribosyl stereochemical configuration.

As used herein, “2′-OMe nucleoside” means a nucleoside comprising a 2′-OMe sugar moiety.

As used herein, “2′-F” means a 2′-fluoro group in place of the 2′-OH group of a furanosyl sugar moiety. A “2′-F sugar moiety” means a sugar moiety with a 2′-F group in place of the 2′-OH group of a furanosyl sugar moiety. Unless otherwise indicated, a 2′-F sugar moiety is in the β-D-ribosyl configuration.

As used herein, “2′-F nucleoside” means a nucleoside comprising a 2′-F sugar moiety.

As used herein, “2′-substituted nucleoside” means a nucleoside comprising a 2′-substituted furanosyl sugar moiety. As used herein, “2′-substituted” in reference to a sugar moiety means a sugar moiety wherein at least one 2′-substituent group is other than H and OH. For example, an antisense oligomeric compound (e.g., RNAi agent) provided herein comprises one or more modified sugar moiety wherein at least one modified sugar moiety comprises a 2′-substituted nucleoside wherein the 2′-substituent group is independently selected from 2′-F, 2′-MOE, 2′-OMe, and cEt.

As used herein, “5-methylcytosine” means a cytosine modified with a methyl group attached at the 5 position. A 5-methylcytosine is a modified nucleobase.

As used herein, “abasic sugar moiety” means a modified nucleoside wherein a sugar moiety of a nucleoside is not attached to a nucleobase. Abasic sugar moieties are sometimes referred to in the art as “abasic nucleosides.”

As used herein, “ameliorate” means improvement in or lessening of at least one symptom of an associated disease, disorder or condition. In certain embodiments, amelioration is reduction in severity or frequency of a symptom or the delayed onset or slowing of progression in the severity or frequency of a symptom. Progression or severity of indicators may be determined by subjective or objective measures known in the art.

As used herein, “antisense activity” means any detectable and/or measurable change attributable (whether directly and/or indirectly) to hybridization of an antisense agent to its target nucleic acid. In certain embodiments, antisense activity is a decrease in the amount or expression of a target nucleic acid or protein encoded by such target nucleic acid compared to target nucleic acid levels or target protein levels in the absence of the antisense agent. In certain embodiments, agents have antisense activity when they reduce or inhibit the amount or activity of a target nucleic acid by 25% or more in an in vitro assay. In certain embodiments, agents have antisense activity when they reduce or inhibit the amount or activity of a target nucleic acid by 25% or more in an in vivo assay. In certain embodiments antisense activity is assessed in a standard assay.

As used herein, “antisense agent” means an antisense oligonucleotide and optionally one or more additional features, e.g., a paired oligonucleotide, a conjugate group and/or a terminal group. As used herein, “antisense oligonucleotide” means an oligonucleotide that is capable of hybridizing to a target nucleic acid and is capable of at least one antisense activity. In certain embodiments, antisense agents selectively affect one or more target nucleic acid. In certain embodiments, an antisense agent is a modified oligonucleotide provided herein that is capable of hybridizing to a target nucleic acid and is capable of at least one antisense activity. An antisense oligonucleotide may be paired with a second oligonucleotide (herein, a “sense oligonucleotide”) that is complementary to the antisense oligonucleotide (that is capable of hybridizing to an antisense oligonucleotide to form a double-stranded antisense oligonucleotide, a duplex antisense oligonucleotide) or may be an unpaired antisense oligonucleotide (a singled-stranded antisense oligonucleotide). As used herein, “sense agent” means a sense oligonucleotide and optionally one or more additional features, such as a conjugate group.

As used herein, “bicyclic nucleoside” or “BNA” means a nucleoside comprising a bicyclic sugar moiety. As used herein, “bicyclic sugar” or “bicyclic sugar moiety” means a modified sugar moiety comprising two rings, wherein the first ring of the bicyclic sugar moiety is a furanosyl ring and the second ring is formed via a bridge connecting two of the atoms in the first ring thereby forming a bicyclic structure.

Examples of bicyclic sugar moieties include LNA (locked nucleic acid) sugar moiety and cEt sugar moiety as defined herein.

As used herein, “blunt” or “blunt ended” in reference to an oligomeric duplex means that both strands are the same length and there are no terminal unpaired nucleotides on either strand (i.e. no overhanging nucleotides). One or both ends of an oligomeric duplex can be blunt.

As used herein, “cell-targeting moiety” means a conjugate group or portion of a conjugate group that is capable of binding to a particular cell type or particular cell types. In certain embodiments, a cell-targeting moiety binds to a cell surface moiety, such as a cell surface receptor on a particular cell type.

As used herein, “cleavable moiety” means a bond or group of atoms that is cleaved following administration to a subject. In certain embodiments, a cleavable moiety cleaved inside a cell or sub-cellular compartment, such as an endosome or lysosome. In certain embodiments, a cleavable moiety may be cleaved by endogenous enzymes, such as nucleases.

As used herein, “complementary” in reference to an oligonucleotide or region thereof means that at least 70% of the nucleobases of such oligonucleotide or region thereof and the nucleobases of another nucleic acid or region thereof are capable of hydrogen bonding with one another when the nucleobase sequence of the oligonucleotide or region and the other nucleic acid are aligned in opposing directions. As used herein, “complementary nucleobases” means nucleobases that are capable of forming hydrogen bonds with one another. Complementary nucleobase pairs include adenine (A) and thymine (T); adenine (A) and uracil (U); cytosine (C) and guanine (G); and 5-methylcytosine (^{m}C) and guanine (G). Certain modified nucleobases that pair with unmodified nucleobases or with other modified nucleobases are known in the art. For example, inosine can pair with cytosine or uracil. Complementary oligonucleotides and/or nucleic acids need not have nucleobase complementarity at each nucleoside. Rather, some mismatches are tolerated. As used herein, “fully complementary” or “100% complementary” in reference to an oligonucleotide, or a region thereof, means that the oligonucleotide, or region thereof, is complementary to another oligonucleotide or nucleic acid at each nucleobase of the shorter of the two molecules, or at each nucleoside if in reference to oligonucleotides that are the same length.

As used herein, “complementary region” in reference to a nucleic acid sequence is the range of nucleobases of the nucleic acid sequence that is complementary with a second nucleic acid sequence (e.g., an oligonucleotide or target nucleic acid).

As used herein, “constrained ethyl” or “cEt” or “cEt sugar moiety” means a R-D ribosyl bicyclic sugar moiety wherein the second ring of the bicyclic sugar is formed via a bridge connecting the 4′-carbon and the 2′-carbon of the β-D ribosyl sugar moiety, wherein the bridge has the formula 4′-CH(CH₃)—O-2′, and wherein the methyl group of the bridge is in the S configuration.

As used herein, “cEt nucleoside” means a nucleoside comprising a cEt sugar moiety.

As used herein, “hybridization” means the process of two complementary nucleic acid sequences (e.g., oligonucleotides, nucleic acids) annealing or bonding together to form a duplex or double stranded structure. While not limited to a particular mechanism, the most common mechanism of hybridization involves hydrogen bonding, which may be Watson-Crick, Hoogsteen or reversed Hoogsteen hydrogen bonding, between complementary nucleobases. In certain embodiments, complementary nucleic acid sequences in separate molecules include, but are not limited to, an antisense agent and a nucleic acid target. In certain embodiments, complementary nucleic acid sequences in separate molecules include, but are not limited to, an oligonucleotide and a nucleic acid target. In certain embodiments, complementary nucleic acid sequences in separate molecules include, but are not limited to, an antisense agent and a sense agent. In certain embodiments, complementary nucleic acid sequences in a same molecule includes, but is not limited to, an oligomeric compound comprising oligonucleotides (e.g., a hairpin oligo).

As used herein, “internucleoside linkage” is the covalent linkage between adjacent nucleosides in an oligonucleotide. As used herein “modified internucleoside linkage” means any internucleoside linkage other than a phosphodiester internucleoside linkage. A “phosphorothioate internucleoside linkage” is a modified internucleoside linkage in which one of the non-bridging oxygen atoms of a phosphodiester internucleoside linkage is replaced with a sulfur atom. Unless otherwise indicated, and in the context of linked nucleosides each comprising a furanosyl sugar moiety, an internucleoside linkage joins the 3′-carbon of one furanosyl sugar moiety to the 5′-carbon of the other furanosyl sugar moiety.

As used herein, “inverted nucleoside” means a nucleoside having a 3′ to 3′ and/or 5′ to 5′ internucleoside linkage, as shown herein. As used herein, “inverted sugar moiety” means the sugar moiety of an inverted nucleoside or an abasic sugar moiety having a 3′ to 3′ and/or 5′ to 5′ internucleoside linkage.

As used herein, “linked nucleosides” are nucleosides that are connected in a contiguous sequence (i.e., no additional nucleosides are presented between those that are linked).

As used herein, “linker-nucleoside” means a nucleoside that links, either directly or indirectly, an oligonucleotide to a conjugate moiety. When present in an agent, linker-nucleosides are located within the conjugate linker of an agent. Linker-nucleosides are not considered part of the oligonucleotide portion of an agent even if they are contiguous with the oligonucleotide.

As used herein, “mismatch” means a nucleobase at a specified position of a first nucleic acid sequence that is not complementary with the corresponding nucleobase of a second nucleic acid sequence when the first and second nucleic acid sequences are aligned in opposing directions.

As used herein, “modified nucleoside” means a compound or subunit comprising a sugar moiety or sugar surrogate and optionally a nucleobase, wherein the sugar moiety is modified, replaced with a sugar surrogate and/or the nucleobase is modified or absent. Modified nucleosides include abasic nucleosides and sugar surrogates.

As used herein, a “modified nucleobase” means a nucleobase other than unmodified A, T, C, U, or G. A “5-methylcytosine” is a modified nucleobase. Inosine (I) is a nucleoside comprising the modified nucleobase hypoxanthine.

As used herein, “motif” means the pattern of unmodified and/or modified sugar moieties, nucleobases, and/or internucleoside linkages, in an oligonucleotide.

As used herein, “non-bicyclic modified sugar moiety” means a modified sugar moiety comprising a modification, such as a substituent, that does not form a bridge between two atoms of the sugar to form a second ring.

As used herein, “nucleobase” means an unmodified nucleobase or a modified nucleobase. As used herein an “unmodified nucleobase” is unmodified adenine (A), unmodified thymine (T), unmodified cytosine (C), unmodified uracil (U), or unmodified guanine (G). As used herein, a “modified nucleobase” is a group of atoms other than unmodified A, T, C, U, or G capable of pairing with at least one unmodified nucleobase. A “5-methylcytosine” is a modified nucleobase.

As used herein, “the nucleobase sequence of” a reference SEQ ID NO, refers only to the order of contiguous nucleobases provided in such SEQ ID NO, independent of any sugar or internucleoside linkage modifications and therefore, unless otherwise indicated, includes compounds wherein each sugar moiety and each internucleoside linkage, independently, is modified or unmodified, irrespective of the presence or absence of modifications, indicated in the referenced SEQ ID NO.

As used herein, “nucleoside overhang” or “overhang” refers to unpaired nucleosides at either or both ends of an oligomeric duplex.

As used herein, “nucleoside” means a compound or fragment of a compound comprising a nucleobase and a sugar moiety. The nucleobase and the sugar moiety of each nucleoside are each, independently, unmodified or modified.

As used herein, “oligomeric agent” means a compound or complex comprising or consisting of a modified oligonucleotide and optionally one or more additional associated features, e.g., an additional modified or unmodified oligonucleotide, one or more conjugate group(s), one or more terminal group(s).

As used herein, “oligomeric compound” means a compound comprising an oligonucleotide and optionally one or more covalently linked chemical features selected from one or more conjugate group and one or more terminal group.

The term “oligomeric duplex” means a duplex formed by two separate complementary oligomeric compounds. Each oligomeric compound of an oligomeric duplex may be referred to as a “duplexed oligomeric compound.”

As used herein, “oligonucleotide” means a strand of linked nucleosides connected via internucleoside linkages, wherein each nucleoside and/or each internucleoside linkage of the strand of linked nucleosides may independently be modified or unmodified. Unless otherwise indicated, oligonucleotides consist of 12-50 linked nucleosides. As used herein, “modified oligonucleotide” means an oligonucleotide comprising one or more modified nucleosides and/or having one or more modified internucleoside linkages. As used herein, “unmodified oligonucleotide” means an oligonucleotide that does not comprise any nucleoside modifications or internucleoside modifications. An oligonucleotide may be paired with a second oligonucleotide that is complementary to the oligonucleotide or it may be unpaired. As used herein, “single-stranded” in reference to a nucleic acid (e.g., an oligonucleotide) means that the nucleic acid (or region thereof) is unpaired and is not part of a duplex, not double stranded. Single-stranded nucleic acids (e.g., oligonucleotides) are capable of hybridizing with complementary nucleic acids to form duplexes, at which point they are no longer single-stranded. As used herein, “duplex” means a structure formed by two separate nucleic acid molecules or portions thereof (e.g., two separate oligonucleotides), at least a portion of which are complementary and that are hybridized to one another but are not covalently bonded to one another. As used herein, “double-stranded” refers to a region of hybridized oligonucleotide(s). A double-stranded oligonucleotide means either two separate oligonucleotides that are hybridized to one another (a duplex) or a single molecule that has folded onto itself (e.g., a hairpin structure). In certain embodiments, such double-strand results from hybridization of an oligonucleotide (or portion thereof) to a target region of a transcript. In certain embodiments, a double-strand results from hybridization of two oligonucleotides (or portions thereof) to one another. In certain embodiments, the hybridized regions are portions (including the entirety) of two separate molecules (e.g., no covalent bond connects the two complementary strands together). In certain embodiments, the hybridized regions are portions of the same molecule that have hybridized (e.g., a hairpin structure).

As used herein “pharmaceutical composition” means a mixture of substances suitable for administering to a subject. For example, a pharmaceutical composition may comprise an agent (e.g., an oligomeric agent, duplex, or antisense agent) and a sterile aqueous solution. In certain embodiments, a pharmaceutical composition shows activity in certain cell lines.

As used herein, “pharmaceutically acceptable carrier or diluent” means an ingredient in a pharmaceutical composition suitable for use in administering to a subject. Typically, a “carrier” or “diluent” lacks pharmacological activity but is necessary or desirable in preparing a composition. For example, a diluent in an injected composition can be a liquid, e.g., PBS, or saline solution. Certain carriers or diluents enable pharmaceutical compositions to be formulated as, for example, tablets, pills, dragees, capsules, liquids, gels, syrups, slurries, suspension and lozenges for the oral ingestion by a subject. In certain embodiments, a pharmaceutically acceptable carrier or diluent is sterile water, sterile saline, or sterile buffer solution.

As used herein, “pharmaceutically acceptable salts” means physiologically and pharmaceutically acceptable salts of compounds. Pharmaceutically acceptable salts retain the desired biological activity of the parent compound and do not impart undesired toxicological effects thereto.

As used herein, “reduced fluorine content” with respect to a contiguous sequence of linked nucleosides, for example, a modified oligonucleotide (including, for example an antisense oligomeric compound and a sense oligomeric compound) refers to a contiguous linked sequence of nucleosides in which fewer than 25% of the nucleosides contain a sugar moiety that includes a fluorine atom, e.g., a 2′-fluoro sugar moiety. In certain embodiments, no more than 25%, no more than 22%, no more than 20%, no more than 18%, no more than 16%, no more than 15%, no more than 14%, no more than 13%, no more than 12%, no more than 11%, no more than 10%, no more than 9%, no more than 8%, no more than 7%, no more than 6%, no more than 5%, or no more than 4% of the nucleosides in the contiguous linked sequence of nucleosides contain a 2′-fluoro sugar moiety. In certain embodiments, fewer than fewer than 30%, fewer than 25%, fewer than 22%, fewer than 20%, fewer than 18%, fewer than 16%, fewer than 15%, fewer than 14%, fewer than 13%, fewer than 12%, fewer than 11%, fewer than 10%, fewer than 9%, fewer than 8%, or fewer than 7%, or fewer than 6%, or fewer than 5%, of the nucleosides in the contiguous linked sequence of nucleosides contain a 2′-fluoro sugar moiety. “Reduced fluorine content” when referring to the total fluorine content of a double-stranded or duplex nucleic acid refers to a double-stranded or duplex nucleic acid in which fewer than 50% of the total nucleosides (i.e., all the nucleosides contained in both strands) of the nucleic acid contain a sugar moiety containing a fluorine atom, e.g., a 2′-fluoro sugar moiety. In certain embodiments, no more than 25%, no more than 22%, no more than 20%, no more than 18%, no more than 16%, no more than 15%, no more than 14%, no more than 13%, no more than 12%, no more than 11%, no more than 10%, no more than 9%, no more than 8%, no more than 7%, no more than 6%, no more than 5%, or no more than 4% of the total nucleosides in the double-stranded or duplex nucleic acid contain a 2′-fluoro sugar moiety. In certain embodiments, fewer than 35%, fewer than 30%, fewer than 25%, fewer than 22%, fewer than 20%, fewer than 18%, fewer than 15%, fewer than 12%, fewer than 10%, fewer than 9%, fewer than 8%, or fewer than 7% of the total nucleosides in the double-stranded or duplex nucleic acid contain a sugar moiety contain a 2′-fluoro sugar moiety.

As used herein, “RNAi agent” means an antisense agent that acts, at least in part, through RISC or Ago2 to modulate a target nucleic acid and/or protein encoded by a target nucleic acid. RNAi agents include, but are not limited to double-stranded siRNA, single-stranded RNAi (ssRNAi), and microRNA mimics. RNAi agents may comprise conjugate groups and/or terminal groups. In certain embodiments, an RNAi agent modulates the amount and/or activity, of a target nucleic acid. The term RNAi agent excludes antisense agents that act through RNase H.

As used herein, “stabilized phosphate group” means a 5′-phosphate analog that is metabolically more stable than a 5′-phosphate as naturally occurs on DNA or RNA.

As used herein, “stereorandom” or “stereorandom chiral center” in the context of a population of molecules of identical molecular formula means a chiral center that is not controlled during synthesis, or enriched following synthesis, for a particular absolute stereochemical configuration. The stereochemical configuration of a chiral center is random when it is the result of a synthetic method that is not designed to control the stereochemical configuration. For example, in a population of molecules comprising a stereorandom chiral center, the number of molecules having the (S) configuration of the stereorandom chiral center may be the same as the number of molecules having the (R) configuration of the stereorandom chiral center (“racemic”). In certain embodiments, the stereorandom chiral center is not racemic because one absolute configuration predominates following synthesis, e.g., due to the action of non-chiral reagents near the enriched stereochemistry of an adjacent sugar moiety. In certain embodiments, the stereorandom chiral center is at the phosphorous atom of a stereorandom phosphorothioate internucleoside linkage.

As used herein, “subject” means a human or non-human animal. In certain embodiments, the subject is a human.

As used herein, “sugar moiety” means an unmodified sugar moiety or a modified sugar moiety. As used herein, “unmodified sugar moiety” means a 2′-OH(H) β-D-ribosyl sugar moiety, as found in RNA (an “unmodified RNA sugar moiety”). Unmodified sugar moieties have one hydrogen at each of the 1′, 3′, and 4′ positions, an oxygen at the 3′ position, and two hydrogens at the 5′ position. As used herein, “modified sugar moiety” or “modified sugar” means a modified furanosyl sugar other than β-D-ribosyl sugar moiety (the sugar moiety of unmodified RNA), bicyclic sugar moieties, and substituted sugar moieties; and also includes sugar surrogates. Modified sugar moieties may differ from an unmodified RNA sugar moiety by having different substituent(s) (e.g., 2′-F, 2′-MOE, cEt, etc.), having a 2′-deoxy sugar moiety, bicyclic sugar and/or may differ by stereochemistry (e.g., a 2′-α-L-deoxyribosyl sugar moiety). In certain embodiments, modified sugar moieties differ from an unmodified RNA sugar moiety by having both different chemistry (e.g., different substituent(s), 2′-deoxy sugar moiety) and different stereochemistry.

As used herein, “sugar surrogate” means a moiety that can link a nucleobase to another group, such as an internucleoside linkage, conjugate group, or terminal group in an oligonucleotide, but which is not a furanosyl sugar moiety (modified or unmodified) or a bicyclic sugar moiety. Modified nucleosides comprising sugar surrogates can be incorporated into one or more positions within an oligonucleotide and such oligonucleotides are capable of hybridizing to complementary oligomeric compounds or target nucleic acids.

As used herein, “symptom” means any physical feature, manifestation, sign, test result or indication of a disease or disorder, and include subjective and objective indicia of a disease that may be perceived, experienced, detected, observed, measured, and/or quantified. In certain embodiments a symptom is an absence of a feature, such as failing to reach expected developmental milestones. In certain embodiments, a symptom is apparent to a subject or to a medical professional examining or testing said subject. In certain embodiments, a symptom is apparent upon diagnostic testing, including, but not limited to, post-mortem tests. Symptoms may include episodes of abdominal pain, physical fatigue, difficulty thinking, diarrhea, recurrent acute pancreatitis, eruptive cutaneous xanthomata, and hepatosplenomegaly or a combination thereof. In certain embodiments a symptom or collection of symptoms may be considered a hallmark of a cardiovascular disease or disorder.

As used herein, “target nucleic acid” means an APOC3 nucleic acid that an antisense agent is designed to affect. As used herein, “target RNA” means an APOC3 RNA transcript and includes pre-mRNA and/or mRNA unless otherwise specified or specifically relevant (e.g., intron sequence in pre-mRNA). As used herein, “target region” means a portion of an APOC3 target nucleic acid to which an agent (e.g., a modified oligonucleotide, an antisense agent) is designed to hybridize.

As used herein, “treat” “treating” or “treatment” with respect to a disease, means administering an agent as described herein to a subject having or at risk for developing such disease. In certain embodiments treating a disease with a provided agent provided herein results in amelioration of at least one symptom of such disease. In certain embodiments treatment reduces, improves, and/or prevents one or more symptom(s) such that a symptom of the disease is diminished or is not apparent, or may delay development or progression of a subject's disease, disorder or condition or injury. In certain embodiments, treating a subject improves a symptom relative to the same symptom in the absence of treatment. In certain embodiments, treatment reduces the severity or frequency of a symptom, or delays onset of a symptom, slows the progression of a symptom, or slows severity or frequency of a symptom.

### EMBODIMENTS

1. An oligomeric duplex comprising a first oligomeric compound and a second oligomeric compound, wherein:
(1) a first oligomeric compound comprises a first modified oligonucleotide consisting of 18 to 50 linked nucleosides, wherein the nucleobase sequence of the first modified oligonucleotide comprises at least 18, at least 19, at least 20, at least 21, at least 22, or at least 23 contiguous nucleobases of the nucleobase sequence of any one of SEQ ID NOs: 2-5, wherein each of the nucleosides of the first modified oligonucleotide comprises a modified sugar moiety or sugar surrogate and wherein no more than 22%, no more than 20%, no more than 18%, no more than 15%, no more than 10%, or no more than 5% of the modified nucleosides in the first modified oligonucleotide comprises a fluorine; and
(2) a second oligomeric compound comprises a second modified oligonucleotide consisting of 18 to 50 linked nucleosides wherein the nucleobase sequence of the second modified oligonucleotide comprises at least 16, at least 17, at least 18, at least 19, at least 20, or at least 21 contiguous nucleobases of the nucleobase sequence of any one of SEQ ID NOs: 6-8, wherein each of the nucleosides of the second modified oligonucleotide comprises a modified sugar moiety or sugar surrogate and wherein no more than 25%, no more than 20%, no more than 18%, no more than 16%, no more than 14%, no more than 12%, or no more than 10%, of the modified nucleosides in the second modified oligonucleotide comprises a fluorine.
   2. The oligomeric duplex of embodiment 1, wherein at least one modified nucleoside and no more than four modified nucleosides of the first modified oligonucleotide comprises a 2′-F modification.
   3. The oligomeric duplex of embodiment 1 or embodiment 2, wherein at least one modified nucleoside and no more than four modified nucleosides of the second modified oligonucleotide comprises a 2′-F modification.
   4. The oligomeric duplex of any one of embodiments 1-3, wherein each of the nucleosides of the first modified oligonucleotide independently and the second modified oligonucleotide independently comprises a modified sugar moiety or sugar surrogate independently selected from 2′-F, 2′-MOE, 2′-OMe, AND 2′-deoxyribosyl.
   5. The oligomeric duplex of any one of embodiments 1-4, wherein no more than three nucleosides, no more than four nucleosides, no more than five nucleosides, no more than six nucleosides, no more than seven nucleosides, or no more than eight nucleosides in the oligomeric duplex comprise a modified sugar moiety comprising a fluorine or a sugar surrogate comprising a fluorine.
   6. The oligomeric duplex of any one of embodiments 1-5, wherein no more than 22%, no more than 20%, no more than 18%, no more than 16%, no more than 14%, no more than 12%, no more than 10%, or no more than 7%, of the modified nucleosides in the oligomeric duplex comprise a modified sugar moiety comprising a fluorine or a sugar surrogate comprising a fluorine.
   7. The oligomeric duplex of any one of embodiments 1-6, wherein a nucleoside comprising a modified sugar moiety or sugar surrogate comprising a fluorine of the first modified oligonucleotide is independently selected from one of:
i. the second nucleoside counting from the 5′ end,
ii. the second and fourteenth nucleosides counting from the 5′ end, or
iii. the second, fourteenth and sixteenth nucleosides counting from the 5′ end, or
iv. the second, sixth, fourteenth, and sixteenth nucleosides counting from the 5′ end;
wherein each modified sugar moiety or sugar surrogate comprising a fluorine is independently a 2′-fluoro sugar moiety or a 3′-fluoro-hexitol sugar moiety.
   8. The oligomeric duplex of any one of embodiments 1-7, wherein no more than one or no more than two of the modified sugar moiety and/or sugar surrogate in the first modified oligonucleotide comprises a 2′-F modification.
   9. The oligomeric duplex of any one of embodiments 1-8, wherein one or more nucleosides of the first modified oligonucleotide is a 2′-deoxynucleoside.
   10. The oligomeric duplex of embodiment 9, wherein the one or more 2′-deoxynucleosides is one or more nucleosides in a region of the sequence of the first modified oligonucleotide between and including the fifth nucleoside to the sixteenth nucleoside counting from the 5′ end of the first modified oligonucleotide.
   11. The oligomeric duplex of embodiment 10, wherein the one or more 2′-deoxynucleosides is in a region of the sequence of the first modified oligonucleotide that is any of the sixth, fourteenth, and/or sixteenth nucleosides counting from the 5′ end of the first modified oligonucleotide.
   12. The oligomeric duplex of any one of embodiments 9-11, wherein fewer than 10%, or fewer than 5%, of the nucleosides of the first modified oligonucleotide comprises a 2′-F modification.
   13. The oligomeric duplex of any one of embodiments 12, wherein the one or more 2′-deoxynucleosides is the sixth, fourteenth, and/or sixteenth nucleoside counting from the 5′ end of the first modified oligonucleotide.
   14. The oligomeric duplex of embodiment 13, wherein only one nucleoside or only three nucleosides of the first modified oligonucleotide are 2′-deoxynucleoside.
   15. The oligomeric duplex of embodiment 14, wherein the one 2′-deoxynucleoside is the sixth nucleoside, or the three nucleosides are the sixth, the fourteenth and the sixteenth nucleosides, counting from the 5′ end of the first modified oligonucleotide.
   16. The oligomeric duplex of embodiment 14, wherein the one 2′-deoxynucleoside is the sixth nucleoside counting from the 5′ end of the first modified oligonucleotide.
   17. The oligomeric duplex of embodiments 14, wherein the three nucleosides are the sixth, the fourteenth and the sixteenth nucleosides, counting from the 5′ end of the first modified oligonucleotide.
   18. The oligomeric duplex of embodiment 17, wherein each of the fourteenth and sixteenth nucleoside counting from the 5′ end of the first modified oligonucleotide is a 2′-deoxyuridine.
   19. The oligomeric duplex of embodiment 17, wherein each of the fourteenth and sixteenth nucleoside counting from the 5′ end of the first modified oligonucleotide is a 2′-deoxythymidine.
   20. The oligomeric duplex of any one of embodiments 1-19, wherein two of the 3′ terminal nucleosides of the first modified oligonucleotide comprise a two nucleoside overhang.
   21. The oligomeric duplex of embodiment 20, wherein the overhang nucleosides comprise two modified adenosine (AA) two modified uridine (UU) nucleosides, two modified inosine (II) nucleosides, or two modified nucleosides wherein one is an inosine and one is an adenosine (AI or IA).
   22. The oligomeric duplex of any one of embodiments 1-21, wherein one or more of the nucleosides of the first modified oligonucleotide comprises a 2′-OMe sugar moiety.
   23. The oligomeric duplex of embodiment 22, wherein at least 50%, at least 60%, at least 65%, at least 70%, at least 75% or at least 80% of the nucleosides of the first modified oligonucleotide comprise a 2′-OMe sugar moiety.
   24. The oligomeric duplex of embodiment 22 or embodiment 23, wherein the one or more nucleosides comprising a 2′-OMe sugar moiety are in a region of the sequence of the first modified oligonucleotide between and including the third and twenty-third nucleosides counting from the 5′ end of the first modified oligonucleotide.
   25. The oligomeric duplex of embodiment 24, wherein at least thirteen nucleosides, at least fourteen nucleosides, at least fifteen nucleosides, at least sixteen nucleosides, at least seventeen nucleosides, at least eighteen nucleosides, at least nineteen nucleosides, or at least twenty nucleosides of the first modified oligonucleotide comprise a 2′-OMe sugar moiety.
   26. The oligomeric duplex of any one of embodiments 1-25, wherein one or more of the nucleosides of the first modified oligonucleotide comprise a 2′-MOE sugar moiety.
   27. The oligomeric duplex of embodiment 26, wherein the 5′- and/or 3′-terminal nucleosides of the first modified oligonucleotide comprise a 2′-MOE sugar moiety.
   28. The oligomeric duplex of embodiment 27, wherein the nucleoside immediately 5′ of the 3′-terminal nucleoside of the first modified oligonucleotide comprises a 2′-MOE sugar moiety.
   29. The oligomeric duplex of embodiment 26, wherein at least one nucleoside of the first modified oligonucleotide comprising a 2′-MOE sugar moiety is an internal nucleoside in a region of the sequence of the first modified oligonucleotide that is any of the ninth and/or tenth nucleosides counting from the 5′ end of the first modified oligonucleotide.
   30. The oligomeric duplex of any one of embodiments 1-29, wherein the first oligomeric compound comprises a stabilized phosphate group attached to the 5′-terminal nucleoside.
   31. The oligomeric duplex of embodiment 30, wherein the stabilized phosphate group comprises a methylene phosphonate, cyclopropyl phosphonate or a vinyl phosphonate.
   32. The oligomeric duplex of any one of embodiments 1-31, wherein the first oligomeric compound comprises at least one modified internucleoside linkage.
   33. The oligomeric duplex of embodiment 32, wherein at least one modified internucleoside linkage is a phosphorothioate internucleoside linkage.
   34. The oligomeric duplex of embodiment 32, wherein
i. fewer than 50%, fewer than 45%, fewer than 40%, or fewer than 35%; and
ii. greater than 10%, greater than 15%, greater than 20%, or greater than 25% of the internucleoside linkages of the first oligomeric compound are modified internucleoside linkages.
   35. The oligomeric duplex of embodiment 32, wherein each internucleoside linkage of the first oligomeric compound is independently selected from a phosphodiester internucleoside linkage and a phosphorothioate internucleoside linkage.
   36. The oligomeric duplex of embodiment 35, wherein the internucleoside linkages between the first and second nucleosides and the second and third nucleosides counting from the 5′ end of the first oligomeric compound are phosphorothioate internucleoside linkages.
   37. The oligomeric duplex of embodiment 35, wherein the internucleoside linkages between the first and second nucleosides and the second and third nucleosides counting from the 3′ end of the first oligomeric compound are phosphorothioate internucleoside linkages.
   38. The oligomeric duplex of embodiment 35, wherein the internucleoside linkages between the first and second nucleosides and the second and third nucleosides counting from the 5′ end of the first oligomeric compound, and the internucleoside linkages between the first and second nucleosides and the second and third nucleosides counting from the 3′ end of the first oligomeric compound are modified internucleoside linkages.
   39. The oligomeric duplex of any one of embodiments 35-38, wherein the first oligomeric compound comprises at least one modified internucleoside linkage in a region of the sequence of the first oligomeric compound between and including the fifth nucleoside to the eighteenth nucleoside counting from the 5′ end of the first oligomeric compound internucleoside linkages.
   40. The oligomeric duplex of embodiment 39, wherein at least one modified internucleoside linkage is in a region of the sequence of the first oligomeric compound that is any of the internucleoside linkage between the sixth and seventh nucleosides, the internucleoside linkage between the fourteenth and fifteenth nucleosides, and/or the internucleoside linkage between the sixteenth and seventeenth nucleosides counting from the 5′ end of the first oligomeric compound.
   41. The oligomeric duplex of any one of embodiments 1-40, wherein the nucleobase sequence of the first oligomeric compound comprises at least 18, at least 19, at least 20, at least 21, at least 22, or at least 23 contiguous nucleobases of any one of the sequences of SEQ ID NOs: 2-5, 11-34, or 51-74.
   42. The oligomeric duplex of any one of embodiments 1-40, wherein the nucleobase sequence of the first oligomeric compound comprises the sequence of any one of SEQ ID NOs: 2-5, 11-34, or 51-74.
   43. The oligomeric duplex of any one of embodiments 1-40, wherein the nucleobase sequence of the first oligomeric compound consists of the sequence of any one of SEQ ID NOs: 2-5, 11-34, or 51-74.
   44. The oligomeric duplex of any one of embodiments 1-40, wherein the first oligomeric compound and the second oligomeric compound each independently consist of 18 to 30, 18 to 25, 18 to 24, 18 to 23, 18-22, 18-21, 18-20, 19 to 30, 19 to 25, 19 to 24, 19 to 23, 19-21, 20 to 30, 20 to 25, 20 to 24, 21 to 23, 20 to 22, or 19, 21, or 23 linked nucleosides.
   45. The oligomeric duplex of any one of embodiments 1-44, wherein no more than 4 nucleosides, no more than 3 nucleosides, or no more than 2 nucleosides in the second modified oligonucleotide comprise a modified sugar moiety comprising a fluorine.
   46. The oligomeric duplex of any one of embodiments 1-44, wherein none of the nucleosides before the seventh or after the eleventh nucleoside counting from the 5′ end of the second modified oligonucleotide comprise a modified sugar moiety comprising a fluorine.
   47. The oligomeric duplex of any one of embodiments 1-44, wherein one or more of the seventh, ninth, tenth, and eleventh nucleosides counting from the 5′ end of the second modified oligonucleotide comprises a modified sugar moiety comprising a fluorine.
   48. The oligomeric duplex embodiment 47, wherein two or more of the seventh, ninth, tenth and eleventh nucleosides counting from the 5′ end of the second modified oligonucleotide comprise a modified sugar moiety comprising a fluorine.
   49. The oligomeric duplex embodiment 48, wherein the ninth and tenth nucleosides or the tenth and eleventh nucleosides counting from the 5′ end of the second modified oligonucleotide comprise a modified sugar moiety comprising a fluorine.
   50. The oligomeric duplex of any one of embodiments 44-49, wherein the modified sugar moiety comprising a fluorine is a 2′-fluoro sugar moiety.
   51. The oligomeric duplex of any one of embodiments 1-50, wherein one or more of the nucleosides of the second modified oligonucleotide comprises a 2′-OMe sugar moiety.
   52. The oligomeric duplex of embodiment 51, wherein at least 50%, or at least 60%, or at least 65%, or at least 70%, or at least 80%, or at least 85%, or at least 90% of the nucleosides of the second modified oligonucleotide comprise a 2′-OMe sugar moiety.
   53. The oligomeric duplex of embodiment 51, wherein at least each of the nucleosides from the region beginning and including the third nucleoside to and including the eighth nucleoside from the 5′-terminal end, and from and including the twelfth nucleoside from the 5′-terminal end to the third nucleoside from the 3′-terminal end of the second modified oligonucleotide comprises a 2′-OMe sugar moiety.
   54. The oligomeric duplex of embodiment 51, wherein any of the 3′-terminal nucleoside, the nucleoside immediately 5′ of the 3′-terminal nucleoside, the 5′-terminal nucleoside, and/or the nucleoside immediately 3′ of the 5′-terminal nucleoside of the second modified oligonucleotide comprises a 2′-OMe sugar moiety.
   55. The oligomeric duplex of embodiment 51, wherein at least each of the nucleosides from the 5′-terminal nucleoside of the second modified oligonucleotide to and including the eighth nucleoside, and the twelfth nucleoside from the 5′-terminal end to the 3′-terminal nucleoside of the second modified oligonucleotide comprises a 2′-OMe sugar moiety.
   56. The oligomeric duplex of any one of embodiments 1-55, wherein one or more of the nucleosides of the second modified oligonucleotide comprise a 2′-MOE sugar moiety.
   57. The oligomeric duplex of embodiment 56, wherein the 3′-terminal nucleoside of the second modified oligonucleotide comprises a 2′-MOE sugar moiety and/or the nucleoside immediately 5′ of the 3′-terminal nucleoside of the second modified oligonucleotide comprises a 2′-MOE sugar moiety.
   58. The oligomeric duplex of embodiment 55, wherein the 5′-terminal nucleoside of the second modified oligonucleotide comprises a 2′-MOE sugar moiety and/or the nucleoside immediately 3′ of the 5′-terminal nucleoside of the second oligomeric compound each comprises a 2′-MOE sugar moiety.
   59. The oligomeric duplex of embodiment 55, wherein the 5′-terminal nucleoside, the nucleoside immediately 3′ of the 5′-terminal nucleoside, the 3′-terminal nucleoside, and the nucleoside immediately 5′ of the 3′-terminal nucleoside of the second modified oligonucleotide comprises a 2′-MOE sugar moiety.
   60. The oligomeric duplex of embodiment 51, wherein each of the nucleosides from the region beginning and including the third nucleoside to and including the eighth nucleoside from the 5′-terminal end, and from and including the twelfth nucleoside from the 5′-terminal end to the third nucleoside from the 3′-terminal end of the second modified oligonucleotide comprises a 2′-OMe sugar moiety; and wherein the 5′-terminal nucleoside, the nucleoside immediately 3′ of the 5′-terminal nucleoside, the 3′-terminal nucleoside, and the nucleoside immediately 5′ of the 3′-terminal nucleoside of the second oligomeric compound each comprises a 2′-MOE sugar moiety.
   61. The oligomeric duplex of any one of embodiments 1-60, wherein the second oligomeric compound comprises at least one modified internucleoside linkage.
   62. The oligomeric duplex of embodiment 61, wherein at least one modified internucleoside linkage is a phosphorothioate internucleoside linkage.
   63. The oligomeric duplex of embodiment 61, wherein fewer than 40%, fewer than 35%, or fewer than 30%; and greater than 10%, greater than 15%, greater than 18%, greater than 20%, or greater than 25% of the internucleoside linkages of the second oligomeric compound are modified internucleoside linkages.
   64. The oligomeric duplex of embodiment 61, wherein each internucleoside linkage of the second oligomeric compound is independently selected from a phosphodiester internucleoside linkage and a phosphorothioate internucleoside linkage.
   65. The oligomeric duplex of embodiment 64, wherein the internucleoside linkages between the first and second nucleosides and the second and third nucleosides counting from the 5′ end of the second oligomeric compound are modified internucleoside linkages.
   66. The oligomeric duplex of embodiment 64, wherein the internucleoside linkages between the first and second nucleosides and the second and third nucleosides counting from the 3′ end of the second oligomeric compound are modified internucleoside linkages.
   67. The oligomeric duplex of embodiment 64, wherein the internucleoside linkages between the first and second nucleosides and the second and third nucleosides counting from the 5′ end of the second oligomeric compound, and the internucleoside linkages between the first and second nucleosides and the second and third nucleosides counting from the 3′ end of the second oligomeric compound are modified internucleoside linkages.
   68. The oligomeric duplex of any one of embodiments 64-67, wherein the second oligomeric compound comprises at least one additional modified internucleoside linkage in a region of the sequence of the second oligomeric compound between and including the ninth nucleoside to the eleventh nucleoside counting from the 5′ end of the second oligomeric compound internucleoside linkages.
   69. The oligomeric duplex of embodiment 68, wherein the at least one additional modified internucleoside linkage is in a region of the sequence of the second oligomeric compound that is any of the internucleoside linkage between the ninth and tenth nucleosides, the internucleoside linkage between the tenth and eleventh nucleosides, and/or the internucleoside linkage between the eleventh and twelfth nucleosides counting from the 5′ end of the second oligomeric compound.
   70. The oligomeric duplex of any one of embodiments 1-69, wherein the nucleobase sequence of the second modified oligonucleotide comprises at least 16, at least 17, at least 18, at least 19, at least 20, or at least 21 contiguous nucleobases of any one of the sequences of SEQ ID NOs: 6-8, 35-49, or 75-89.
   71. The oligomeric duplex of any one of embodiments 1-69, wherein the nucleobase sequence of the second modified oligonucleotide comprises the sequence of any one of SEQ ID NOs: 6-8, 35-49, or 75-89.
   72. The oligomeric duplex of any one of embodiments 1-69, wherein the nucleobase sequence of the second modified oligonucleotide consists of the sequence of any one of SEQ ID NOs: 6-8, 35-49, or 75-89.
   73. The oligomeric duplex of any one of embodiments 1-69, wherein the nucleobase sequence of the first modified oligonucleotide comprises at least 18, at least 19, at least 20, at least 21, at least 22, or at least 23 contiguous nucleobases of any one of the sequences of SEQ ID NOs: 2-5, 11-34, or 51-74; and wherein the nucleobase sequence of the second modified oligonucleotide comprises at least 16, at least 17, at least 18, at least 19, at least 20, or at least 21 contiguous nucleobases of any one of the sequences of SEQ ID NOs: 6-8, 35-49, or 75-89.
   74. The oligomeric duplex of any one of embodiments 1-69, wherein the nucleobase sequence of the first oligomeric compound comprises the sequence of any one of SEQ ID NOs: 2-5, 11-34, or 51-74; and wherein the nucleobase sequence of the second oligomeric compound comprises the sequence of any one of SEQ ID NOs: 6-8, 35-49, or 75-89.
   75. The oligomeric duplex of any one of embodiments 1-69, wherein the nucleobase sequence of the first oligomeric compound consists of the sequence of any one of SEQ ID NOs: 2-5, 11-34, or 51-74; and wherein the nucleobase sequence of the second oligomeric compound consists of the sequence of any one of SEQ ID NOs: 6-8, 35-49, or 75-89.
   76. The oligomeric duplex of any one of embodiments 1-75, wherein the duplex comprises a conjugate group comprising a conjugate moiety and a conjugate linker.
   77. The oligomeric duplex of embodiment 76, wherein the conjugate group comprises a cell-targeting moiety.
   78. The oligomeric duplex of embodiment 77, wherein the conjugate group comprises a liver cell targeting moiety.
   79. The oligomeric duplex of embodiment 78, wherein the duplex comprises a conjugate moiety that binds asialoglycoprotein receptor (ASGPR).
   80. The oligomeric duplex of embodiment 79, wherein the conjugate moiety is selected from a GalNAc moiety.
   81. The oligomeric duplex of embodiment 80, wherein the GalNAc conjugate moiety is selected from Table A.
   82. The oligomeric duplex of embodiment 80, wherein the conjugate group consists of a GalNAc ligand and a conjugate linker.
   83. The oligomeric duplex of embodiment 80, wherein the GalNAc ligand has the structure:

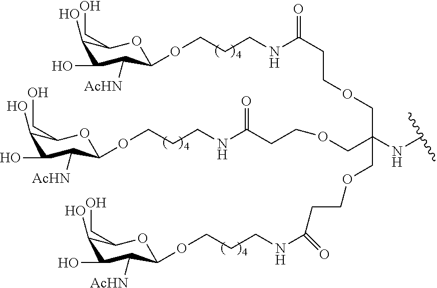

84. The oligomeric duplex of embodiment 83, wherein the conjugate group has the structure:

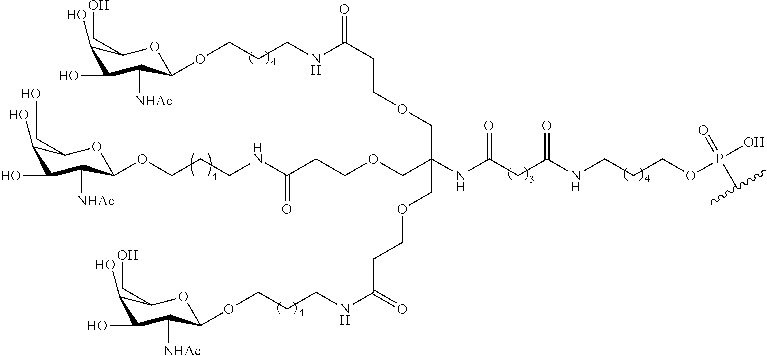

or an ion or salt thereof, wherein the conjugate linker is covalently connected to an oligonucleotide.
85. The oligomeric duplex of embodiment 83, wherein the conjugate group has the structure:

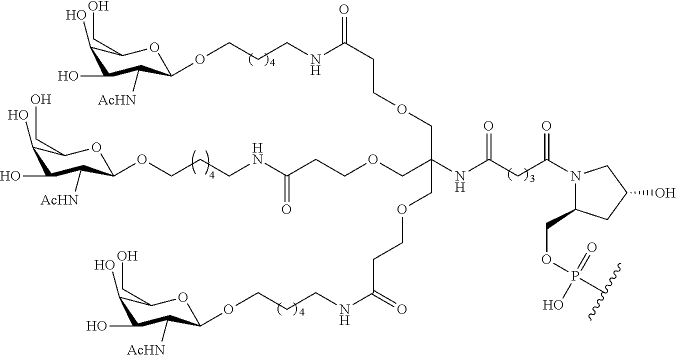

or an ion or salt thereof, wherein the conjugate linker is covalently connected to an oligonucleotide.
86. The oligomeric duplex of any one of embodiments 76-85, wherein the second oligomeric compound comprises the conjugate group conjugated directly to the second modified oligonucleotide.
87. The oligomeric duplex of embodiment 86, wherein the conjugate group is conjugated to the 5′ end or 3′ end of the second modified oligonucleotide.
88. The oligomeric duplex of embodiment 86, wherein the conjugate group is attached to the 5′-terminal nucleoside or the 3′-terminal nucleoside of the second modified oligonucleotide.
89. The oligomeric duplex of embodiment 86, wherein the conjugate group is attached to the 5′-terminal nucleoside of the second modified oligonucleotide.
90. The oligomeric duplex of embodiment 86, wherein the conjugate group is attached to the 3′-terminal nucleoside of the second modified oligonucleotide.
91. The oligomeric duplex of embodiment 82, wherein the conjugate linker of the conjugate group consists of a single bond.
92. The oligomeric duplex of embodiment 82, wherein the conjugate linker of the conjugate group is cleavable.
93. The oligomeric duplex of embodiment 82, wherein the conjugate linker comprises 1 to 3 linker-nucleosides.
94. The oligomeric duplex of embodiment 83, wherein the conjugate group having the structure:

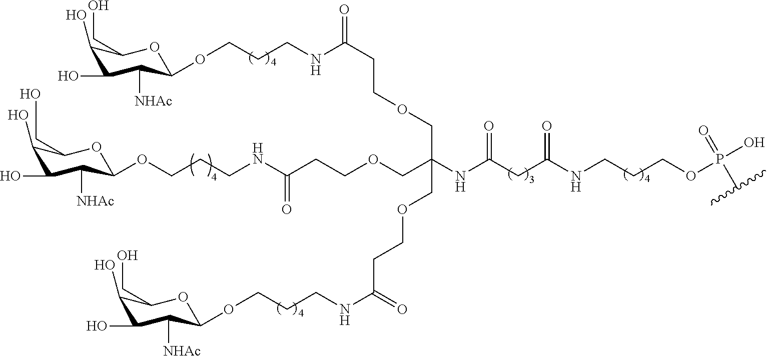

or an ion or salt thereof, is attached to the 5′-terminal nucleoside of the second modified oligonucleotide.
95. The oligomeric duplex of embodiment 83, wherein the conjugate group having the structure:

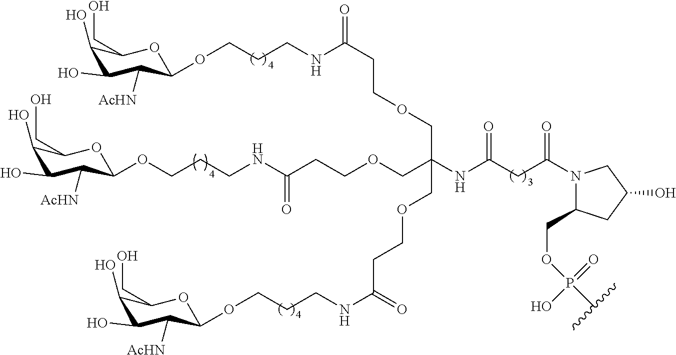

or an ion or salt thereof, is attached to the 3′-terminal nucleoside of the second modified oligonucleotide.
96. An oligomeric agent comprising an oligomeric compound according to any one of the following chemical notation:

(SEQ ID NO: 11)
VP-TesCfsAyoCyoUyoGfoAyoGyoAyoAyoUyoAyoCyoUfoGyoUfoCyoCyoCyoUyoUysUysUy,
(SEQ ID NO: 12)
VP-TesCfsAyoCyoUyoGdoAyoGyoAyoAyoUyoAyoCyoUfoGyoUfoCyoCyoCyoUyoUysUysUy,
(SEQ ID NO: 13)
VP-TesCfsAyoCyoUyoGdsAyoGyoAyoAyoUyoAyoCyoUfoGyoUfoCyoCyoCyoUyoUysUysUy,
(SEQ ID NO: 14)
VP-TesCfsAyoCyoUyoGdoAyoGyoAyoAyoUyoAyoCyoUdoGyoUdoCyoCyoCyoUyoUysUysUy,
(SEQ ID NO: 15)
VP-TesCfsAyoCyoUyoGdsAyoGyoAyoAyoUyoAyoCyoUdsGyoUdsCyoCyoCyoUyoUysUysUy,
(SEQ ID NO: 16)
VP-TesCfsAyoCyoUyoGyoAyoGyoAyoAyoUyoAyoCyoUfoGyoUyoCyoCyoCyoUyoUysUysUy,
(SEQ ID NO: 17)
VP-TesCfsAyoCyoUyoGfoAyoGyoAeoAyoUyoAyoCyoUfoGyoUfoCyoCyoCyoUyoUysUysUy,
(SEQ ID NO: 18)
VP-TesCfsAyoCyoUyoGfoAyoGyoAeoAeoUyoAyoCyoUfoGyoUfoCyoCyoCyoUyoUysUysUy,
(SEQ ID NO: 19)
mP-TesCfsAyoCyoUyoGfoAyoGyoAyoAyoUyoAyoCyoUfoGyoUfoCyoCyoCyoUyoUysUysUy,
(SEQ ID NO: 20)
VP-TesCfsAyoCyoUyoGdsAyoGyoAyoAyoUyoAyoCyoUfoGyoUfoCyoCyoCyoUyoUysAesAe,
(SEQ ID NO: 21)
VP-TesCfsAyoCyoUyoGdsAyoGyoAyoAyoUyoAyoCyoUdsGyoUdsCyoCyoCyoUyoUysAesAe,
(SEQ ID NO: 22)
VP-TesCfsAyoCyoUyoGdoAyoGyoAyoAyoUyoAyoCyoUfoGyoUfoCyoCyoCyoUyoUysAesAe,
(SEQ ID NO: 23)
VP-TesCfsAyoCyoUyoGdoAyoGyoAyoAyoUyoAyoCyoUdoGyoUdoCyoCyoCyoUyoUysAesAe,
(SEQ ID NO: 24)
VP-TesCfsAyoCyoUyoGdsAyoGyoAeoAyoUyoAyoCyoUfoGyoUfoCyoCyoCyoUyoUysAesAe,
(SEQ ID NO: 25)
VP-TesCfsAyoCyoUyoGdoAyoGyoAeoAyoUyoAyoCyoUdoGyoUdoCyoCyoCyoUyoUysAesAe,
(SEQ ID NO: 26)
VP-TesCfsAyoCyoUyoGdoAyoGyoAcoAeoUyoAyoCyoUdoGyoUdoCyoCyoCyoUyoUysAesAe,
(SEQ ID NO: 27)
VP-TesCfsAyoCyoUyoGdoAyoGyoAeoAyoUyoAyoCyoUfoGyoUfoCyoCyoCyoUyoUysAesAe,
(SEQ ID NO: 28)
VP-TesCfsAyoCyoUyoGdoAyoGyoAeoAyoUyoAyoCyoTdoGyoTdoCyoCyoCyoUyoUysAesAe,
(SEQ ID NO: 29)
VP-TesCfsAyoCyoUyoGdsAyoGyoAeoAyoUyoAyoCyoUdsGyoUdsCyoCyoCyoUyoUysAesAe,
(SEQ ID NO: 30)
VP-TesCfsAyoCyoUyoGdoAyoGyoAeoAeoUyoAyoCyoUfoGyoUfoCyoCyoCyoUyoUysAesAe,
(SEQ ID NO: 31)
VP-TesCfsAyoCyoUyoGdsAyoGyoAeoAyoUyoAyoCyoTdsGyoTdsCyoCyoCyoUyoUysAesAe,
(SEQ ID NO: 32)
VP-TesCfsAyoCyoUyoGfoAyoGyoAyoAyoUyoAyoCyoUfoGyoUfoCyoCyoCysAesAe,
(SEQ ID NO: 33)
VP-TesCfsAyoCyoUyoGdsAyoGyoAyoAyoUyoAyoCyoUfoGyoUfoCyoCyoCysAesAe,
and
(SEQ ID NO: 34)
VP-TesCfsAyoCyoUyoGdsAyoGyoAyoAyoUyoAyoCyoUdsGyoUdsCyoCyoCysAesAe,
(SEQ ID NO: 51)
TesCfsAyoCyoUyoGfoAyoGyoAyoAyoUyoAyoCyoUfoGyoUfoCyoCyoCyoUyoUysUysUy,
(SEQ ID NO: 52)
TesCfsAyoCyoUyoGdoAyoGyoAyoAyoUyoAyoCyoUfoGyoUfoCyoCyoCyoUyoUysUysUy,
(SEQ ID NO: 53)
TesCfsAyoCyoUyoGdsAyoGyoAyoAyoUyoAyoCyoUfoGyoUfoCyoCyoCyoUyoUysUysUy,
(SEQ ID NO: 54)
TesCfsAyoCyoUyoGdoAyoGyoAyoAyoUyoAyoCyoUdoGyoUdoCyoCyoCyoUyoUysUysUy,
(SEQ ID NO: 55)
TesCfsAyoCyoUyoGdsAyoGyoAyoAyoUyoAyoCyoUdsGyoUdsCyoCyoCyoUyoUysUysUy,
(SEQ ID NO: 56)
TesCfsAyoCyoUyoGyoAyoGyoAyoAyoUyoAyoCyoUfoGyoUyoCyoCyoCyoUyoUysUysUy,
(SEQ ID NO: 57)
TesCfsAyoCyoUyoGfoAyoGyoAeoAyoUyoAyoCyoUfoGyoUfoCyoCyoCyoUyoUysUysUy,
(SEQ ID NO: 58)
TesCfsAyoCyoUyoGfoAyoGyoAcoAeoUyoAyoCyoUfoGyoUfoCyoCyoCyoUyoUysUysUy,
(SEQ ID NO: 59)
TesCfsAyoCyoUyoGfoAyoGyoAyoAyoUyoAyoCyoUfoGyoUfoCyoCyoCyoUyoUysUysUy,
(SEQ ID NO: 60)
TesCfsAyoCyoUyoGdsAyoGyoAyoAyoUyoAyoCyoUfoGyoUfoCyoCyoCyoUyoUysAesAe,
(SEQ ID NO: 61)
TesCfsAyoCyoUyoGdsAyoGyoAyoAyoUyoAyoCyoUdsGyoUdsCyoCyoCyoUyoUysAesAe,
(SEQ ID NO: 62)
TesCfsAyoCyoUyoGdoAyoGyoAyoAyoUyoAyoCyoUfoGyoUfoCyoCyoCyoUyoUysAesAe,
(SEQ ID NO: 63)
TesCfsAyoCyoUyoGdoAyoGyoAyoAyoUyoAyoCyoUdoGyoUdoCyoCyoCyoUyoUysAesAe,
(SEQ ID NO: 64)
TesCfsAyoCyoUyoGdsAyoGyoAeoAyoUyoAyoCyoUfoGyoUfoCyoCyoCyoUyoUysAesAe,
(SEQ ID NO: 65)
TesCfsAyoCyoUyoGdoAyoGyoAeoAyoUyoAyoCyoUdoGyoUdoCyoCyoCyoUyoUysAesAe,
(SEQ ID NO: 66)
TesCfsAyoCyoUyoGdoAyoGyoAeoAeoUyoAyoCyoUdoGyoUdoCyoCyoCyoUyoUysAesAe,
(SEQ ID NO: 67)
TesCfsAyoCyoUyoGdoAyoGyoAeoAyoUyoAyoCyoUfoGyoUfoCyoCyoCyoUyoUysAesAe,
(SEQ ID NO: 68)
TesCfsAyoCyoUyoGdoAyoGyoAeoAyoUyoAyoCyoTdoGyoTdoCyoCyoCyoUyoUysAesAe,
(SEQ ID NO: 69)
TesCfsAyoCyoUyoGdsAyoGyoAeoAyoUyoAyoCyoUdsGyoUdsCyoCyoCyoUyoUysAesAe,
(SEQ ID NO: 70)
TesCfsAyoCyoUyoGdoAyoGyoAeoAeoUyoAyoCyoUfoGyoUfoCyoCyoCyoUyoUysAesAe,
(SEQ ID NO: 71)
TesCfsAyoCyoUyoGdsAyoGyoAcoAyoUyoAyoCyoTdsGyoTdsCyoCyoCyoUyoUysAesAe,
(SEQ ID NO: 72)
TesCfsAyoCyoUyoGfoAyoGyoAyoAyoUyoAyoCyoUfoGyoUfoCyoCyoCysAesAe,
(SEQ ID NO: 73)
TesCfsAyoCyoUyoGdsAyoGyoAyoAyoUyoAyoCyoUfoGyoUfoCyoCyoCysAesAe,
and
(SEQ ID NO: 74)
TesCfsAyoCyoUyoGdsAyoGyoAyoAyoUyoAyoCyoUdsGyoUdsCyoCyoCysAesAe;

wherein:
A=an adenine nucleobase, C=a cytosine nucleobase, G=a guanine nucleobase, T=a thymine nucleobase, U=a uracil nucleobase, d=a 2′-β-D-deoxyribosyl sugar moiety, e=a 2′-MOE sugar moiety, f=a 2′-fluoro sugar moiety, y=a 2′-OMe sugar moiety, o=a phosphodiester internucleoside linkage,
s=a phosphorothioate internucleoside linkage, VP=a 5′ vinyl phosphonate moiety, and mP=methylene phosphonate.
97. An oligomeric agent comprising an oligomeric compound according to any one of the following chemical notation:

(SEQ ID NO: 35)
THA-GalNAc-AysAysGyoGyoGyoAyoCfoAyoGfoUfoAfoUyoUyoCyoUyoCyoAyoGyoUysGysAy,
(SEQ ID NO: 36)
THA-GalNAc-AysAysGyoGyoGyoAyoCyoAyoGyoUfoAfoUyoUyoCyoUyoCyoAyoGyoUysGysAy,
(SEQ ID NO: 37)
THA-GalNAc-AysAysGyoGyoGyoAyoCyoAfoGyoUfoAyoUfoUyoCyoUyoCyoAyoGyoUysGysAy,
(SEQ ID NO: 38)
THA-GalNAc-AysAysGyoGyoGyoAyoCyoAyoGyoUfsAfoUyoUyoCyoUyoCyoAyoGyoUysGysAy,
(SEQ ID NO: 39)
THA-GalNAc-AesAesGyoGyoGyoAyoCyoAyoGyoUfoAfoUyoUyoCyoUyoCyoAyoGyoUysGesAe,
(SEQ ID NO: 40)
THA-GalNAc-AesAesGyoGyoGyoAyoCyoAyoGyoUfsAfoUyoUyoCyoUyoCyoAyoGyoUysGesAe,
(SEQ ID NO: 41)
THA-GalNAc-AesAesGyoGyoGyoAyoCyoAyoGfsUfoAyoUyoUyoCyoUyoCyoAyoGyoUysGesAe,
(SEQ ID NO: 42)
THA-GalNAc-GysGysGyoAyoCyoAyoGfoUyoAfoUfoUfoCyoUyoCyoAyoGyoUysGysAy,
(SEQ ID NO: 43)
THA-GalNAc-GysGysGyoAyoCyoAyoGfoUyoAfoUyoUyoCyoUyoCyoAyoGyoUysGysAy,
(SEQ ID NO: 44)
THA-GalNAc-GysGysGyoAyoCyoAyoGyoUfoAyoUfoUyoCyoUyoCyoAyoGyoUysGysAy,
(SEQ ID NO: 45)
THA-GalNAc-GysGysGyoAyoCyoAyoGyoUyoAfoUyoUfoCyoUyoCyoAyoGyoUysGysAy,
(SEQ ID NO: 46)
THA-GalNAc-GesGesGyoAyoCyoAyoGyoUyoAyoUfsUfoCyoUyoCyoAyoGyoUysGesAe,
(SEQ ID NO: 47)
THA-GalNAc-GesGesGyoAyoCyoAyoGyoUyoAfsUfoUyoCyoUyoCyoAyoGyoUysGesAe,
(SEQ ID NO: 48)
THA-GalNAc-TdoAysAysGyoGyoGyoAyoCfoAyoGfoUfoAfoUyoUyoCyoUyoCyoAyoGyoUysGysAy,
and
(SEQ ID NO: 49)
AysAysGyoGyoGyoAyoCfoAyoGfoUfoAfoUyoUyoCyoUyoCyoAyoGyoUysGysAy-HPPO-GalNAc;

wherein:

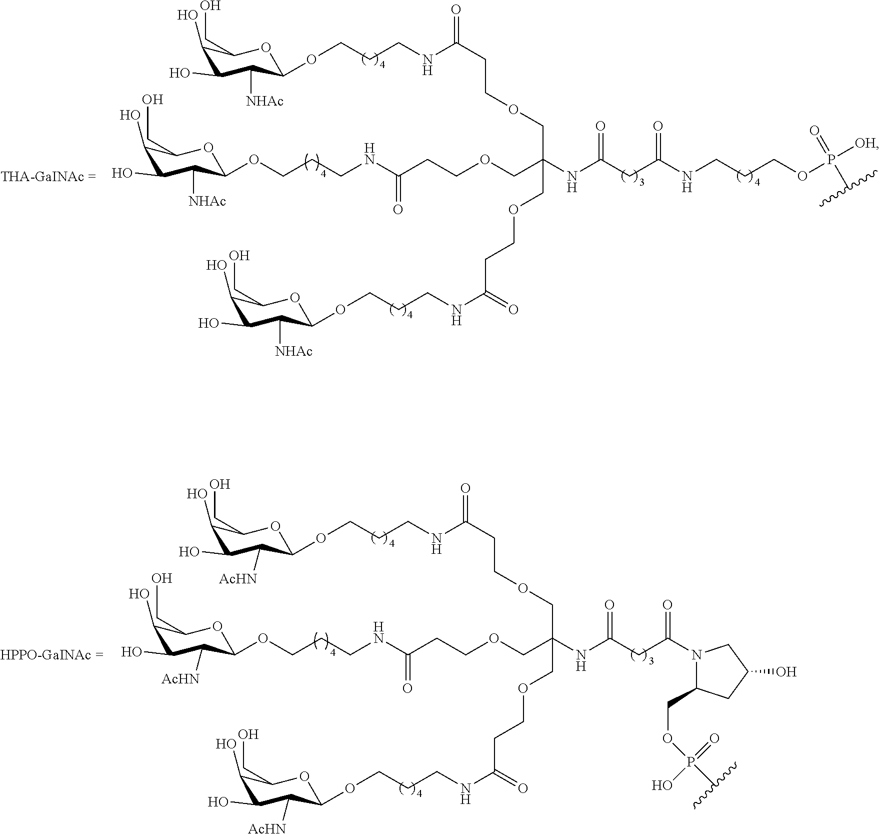

A=an adenine nucleobase, C=a cytosine nucleobase, G=a guanine nucleobase, T=a thymine nucleobase, U=a uracil nucleobase, d=a 2′-β-D-deoxyribosyl sugar moiety, e=a 2′-MOE sugar moiety,
f=a 2′-fluoro sugar moiety, y=a 2′-OMe sugar moiety, o=a phosphodiester internucleoside linkage, and
s=a phosphorothioate internucleoside linkage.
98. An oligomeric agent comprising an oligomeric compound according to any one of the following chemical notation:

(SEQ ID NO: 75)
AysAysGyoGyoGyoAyoCfoAyoGfoUfoAfoUyoUyoCyoUyoCyoAyoGyoUysGysAy,
(SEQ ID NO: 76)
AysAysGyoGyoGyoAyoCyoAyoGyoUfoAfoUyoUyoCyoUyoCyoAyoGyoUysGysAy,
(SEQ ID NO: 77)
AysAysGyoGyoGyoAyoCyoAfoGyoUfoAyoUfoUyoCyoUyoCyoAyoGyoUysGysAy,
(SEQ ID NO: 78)
AysAysGyoGyoGyoAyoCyoAyoGyoUfsAfoUyoUyoCyoUyoCyoAyoGyoUysGysAy,
(SEQ ID NO: 79)
AesAesGyoGyoGyoAyoCyoAyoGyoUfoAfoUyoUyoCyoUyoCyoAyoGyoUysGesAe,
(SEQ ID NO: 80)
AesAesGyoGyoGyoAyoCyoAyoGyoUfsAfoUyoUyoCyoUyoCyoAyoGyoUysGesAe,
(SEQ ID NO: 81)
AesAesGyoGyoGyoAyoCyoAyoGfsUfoAyoUyoUyoCyoUyoCyoAyoGyoUysGesAe,
(SEQ ID NO: 82)
GysGysGyoAyoCyoAyoGfoUyoAfoUfoUfoCyoUyoCyoAyoGyoUysGysAy,
(SEQ ID NO: 83)
GysGysGyoAyoCyoAyoGfoUyoAfoUyoUyoCyoUyoCyoAyoGyoUysGysAy,
(SEQ ID NO: 84)
GysGysGyoAyoCyoAyoGyoUfoAyoUfoUyoCyoUyoCyoAyoGyoUysGysAy,
(SEQ ID NO: 85)
GysGysGyoAyoCyoAyoGyoUyoAfoUyoUfoCyoUyoCyoAyoGyoUysGysAy,
(SEQ ID NO: 86)
GesGesGyoAyoCyoAyoGyoUyoAyoUfsUfoCyoUyoCyoAyoGyoUysGesAe,
(SEQ ID NO: 87)
GesGesGyoAyoCyoAyoGyoUyoAfsUfoUyoCyoUyoCyoAyoGyoUysGesAe,
(SEQ ID NO: 88)
TdoAysAysGyoGyoGyoAyoCfoAyoGfoUfoAfoUyoUyoCyoUyoCyoAyoGyoUysGysAy,
and
(SEQ ID NO: 89)
AysAysGyoGyoGyoAyoCfoAyoGfoUfoAfoUyoUyoCyoUyoCyoAyoGyoUysGysAy,

wherein:
A=an adenine nucleobase, C=a cytosine nucleobase, G=a guanine nucleobase, T=a thymine nucleobase, U=a uracil nucleobase, d=a 2′-β-D-deoxyribosyl sugar moiety, e=a 2′-MOE sugar moiety, f=a 2′-fluoro sugar moiety, y=a 2′-OMe sugar moiety, o=a phosphodiester internucleoside linkage, and
s=a phosphorothioate internucleoside linkage.
99. An oligomeric duplex comprising an oligomeric compound of embodiment 96 and an oligomeric compound of embodiment 97.
100. An oligomeric duplex comprising an oligomeric compound of embodiment 96 and an oligomeric compound of embodiment 98.
101. An oligomeric duplex according to the chemical structure of Compound 1758231 (SEQ ID NO: 31 and SEQ ID NO: 40), or an ion or salt thereof.
102. The oligomeric duplex of embodiment 101, which is the sodium salt or potassium salt.
103. An oligomeric duplex according to the chemical structure of Compound 1758231 sodium salt (SEQ ID NO: 31 and SEQ ID NO: 40).
104. An oligomeric duplex according to the chemical structure of Compound 1755069 (SEQ ID NO: 26 and SEQ ID NO: 40), or an ion or salt thereof.
105. The oligomeric duplex of embodiment 104, which is the sodium salt or potassium salt.
106. An oligomeric duplex according to the chemical structure of Compound 1755069 sodium salt (SEQ ID NO: 26 and SEQ ID NO: 40).
107. An oligomeric duplex according to the chemical structure of Compound 1755072 (SEQ ID NO: 29 and SEQ ID NO: 40), or an ion or salt thereof.
108. The oligomeric duplex of embodiment 107, which is the sodium salt or potassium salt.
109. An oligomeric duplex according to the chemical structure of Compound 1755072 sodium salt (SEQ ID NO: 29 and SEQ ID NO: 40).
110. An oligomeric duplex according to the chemical structure of Compound 1692958 (SEQ ID NO: 11 and SEQ ID NO: 35), or an ion or salt thereof.
111. The oligomeric duplex of embodiment 110, which is the sodium salt or potassium salt.
112. An oligomeric duplex according to the chemical structure of Compound 1692958 sodium salt (SEQ ID NO: 11 and SEQ ID NO: 35).
113. An oligomeric duplex according to the chemical structure of Compound 1754976 (SEQ ID NO: 28 and SEQ ID NO: 40), or an ion or salt thereof.
114. The oligomeric duplex of embodiment 113, which is the sodium salt or potassium salt.
115. An oligomeric duplex according to the chemical structure of Compound 1754976 sodium salt (SEQ ID NO: 28 and SEQ ID NO: 40).
116. An oligomeric duplex according to the chemical structure of Compound 1755063 (SEQ ID NO: 29 and SEQ ID NO: 39), or an ion or salt thereof.
117. The oligomeric duplex of embodiment 116, which is the sodium salt or potassium salt.
118. An oligomeric duplex according to the chemical structure of Compound 1755063 sodium salt (SEQ ID NO: 29 and SEQ ID NO: 39).
119. An oligomeric duplex according to the chemical structure of Compound 1757508 (SEQ ID NO: 23 and SEQ ID NO: 40), or an ion or salt thereof.
120. The oligomeric duplex of embodiment 119, which is the sodium salt or potassium salt.
121. An oligomeric duplex according to the chemical structure of Compound 1757508 sodium salt (SEQ ID NO: 23 and SEQ ID NO: 40).
122. An oligomeric duplex according to the chemical structure of Compound 1758193 (SEQ ID NO: 31 and SEQ ID NO: 39), or an ion or salt thereof.
123. The oligomeric duplex of embodiment 122, which is the sodium salt or potassium salt.
124. An oligomeric duplex according to the chemical structure of Compound 1758193 sodium salt (SEQ ID NO: 31 and SEQ ID NO: 39).
125. A population of oligomeric duplexes or oligomeric agents of any one of embodiments 1-124, wherein the population is enriched for first and/or second oligomeric compounds comprising at least one particular phosphorothioate internucleoside linkage having a particular stereochemical configuration.
126. The population of embodiment 125, wherein the population is enriched for first and/or second oligomeric compounds comprising at least one particular phosphorothioate internucleoside linkage having the (Sp) or (Rp) configuration.
127. The oligomeric duplex or oligomeric agent of any one of embodiments 1-124, wherein the first oligomeric compound consists of 23 linked nucleosides and the second oligomeric compound consists of 21 linked nucleosides.
128. An antisense agent comprising or consisting of an oligomeric duplex or oligomeric agent of any one of embodiments 1-124.
129. The antisense agent of embodiment 128, wherein the antisense agent is an RNAi agent capable of reducing the amount of APOC3 nucleic acid through the activation of RISC/Ago2.
130. A pharmaceutical composition comprising the oligomeric duplex or oligomeric agent of any one of embodiments 1-124 or 127, the population of any one of embodiments 125-126, or the antisense agent of embodiment 128 or embodiment 129, and a pharmaceutically acceptable diluent or carrier.
131. The pharmaceutical composition of embodiment 130, wherein the pharmaceutically acceptable diluent is water or phosphate-buffered saline.
132. The pharmaceutical composition of embodiment 130, wherein the pharmaceutical composition consists essentially of the oligomeric duplex, oligomeric agent or the antisense agent, and water or phosphate-buffered saline.
133. A method of decreasing the amount of APOC3 RNA or ApoCIII protein in a cell, tissue, organ or subject, comprising contacting the cell, tissue, organ or subject with the oligomeric duplex or oligomeric agent of any one of embodiments 1-124, the population of any one of embodiments 125-126, or the antisense agent of embodiment 128 or embodiment 129, or the pharmaceutical composition of any one of embodiments 130-132. 134. The method of embodiment 133, wherein the cell is a liver cell.
135. A method comprising administering to a subject the oligomeric duplex or oligomeric agent of any one of embodiments 1-124, the population of any one of embodiments 125-126, or the antisense agent of embodiment 128 or embodiment 129, or the pharmaceutical composition of any one of embodiments 130-132; wherein the subject has or is at risk for developing an atherosclerotic cardiovascular disease, condition or disorder, hypertriglyceridemia, cardiovascular disease, and/or coronary heart disease.
136. A method of treating a disease, disorder, condition or injury associated with lipoprotein metabolism misregulation, or postponing a symptom of a disease, disorder or condition associated with elevated triglycerides, comprising administering to a subject having, or at risk of having, a disease, disorder or condition associated with lipoprotein metabolism misregulation, a therapeutically effective amount of the oligomeric duplex or oligomeric agent of any one of embodiments 1-124, the population of any one of embodiments 125-126, or the antisense agent of embodiment 128 or embodiment 129, or the pharmaceutical composition of any one of embodiments 130-132; wherein the disease, disorder, condition or injury is selected from a cardiovascular disease, disorder, condition, a metabolic disease, disorder, or condition, or an inflammatory disease disorder or condition.
137. A method of treating a cardiovascular disease, disorder or condition, a metabolic disease, disorder, or condition, and/or an inflammatory disease, disorder or condition in a subject, comprising administering to a subject having, or at risk of having, a cardiovascular, metabolic, and/or inflammatory disease, disorder, condition, an oligomeric duplex or oligomeric agent of any one of embodiments 1-124, the population of any one of embodiments 125-126, or the antisense agent of embodiment 128 or embodiment 129, or the pharmaceutical composition of any one of embodiments 130-132; wherein the disease, disorder, condition or injury is a dyslipidemia, atherosclerotic cardiovascular disease (ASCVD), and/or coronary artery disease (CAD).
138. A method of decreasing the amount of APOC3 RNA and/or ApoCIII protein in the liver of a subject having or at risk of developing a disease, disorder or condition associated with elevated triglycerides, comprising administering to a subject having, or at risk of having, a disease, disorder or condition associated with lipoprotein metabolism misregulation, an oligomeric duplex or oligomeric agent of any one of embodiments 1-124, the population of any one of embodiments 125-126, or the antisense agent of embodiment 128 or embodiment 129, or the pharmaceutical composition of any one of embodiments 130-132; wherein the disease, disorder, condition or injury is a cardiovascular disease, disorder or condition, a metabolic disease, disorder, or condition, and/or an inflammatory disease, disorder or condition.
139. The method of any one of embodiments 135-138, wherein the amount of APOC3 RNA and/or ApoCIII protein in liver and/or plasma of the subject is decreased.
140. The method of any one of embodiments 135-138, wherein the method results in ameliorating (whether by reduced frequency, severity) at least one symptom of a disease, disorder, condition or injury associated with lipoprotein metabolism misregulation.
141. The method of any one of embodiments 135-138, wherein the method results in preventing, delay or postponing, or slowing the development or progression of at least one symptom of a disease, disorder or condition associated with elevated triglycerides.
142. The method of any one of embodiments 135-138, wherein the disease, disorder or condition is hypertriglyceridemia or atherosclerotic cardiovascular disease (ASCVD) or coronary artery disease (CAD).
143. The method of embodiment 142, wherein the ASCVD is ischemic vascular disease (IVD) or ischemic heart disease (IHD).
144. The method of embodiment 142, wherein the hypertriglyceridemia is genetic hypertriglyceridemia or familial chylomicronemia syndrome (FCS) or familial partial lipodystrophy (FPL).
145. The method of any one of embodiments 135-138, wherein the metabolic disease disorder or condition is pancreatitis, diabetes, or insulin insensitivity.
146. The method of embodiment 136, wherein at least one symptom of a disease, disorder or condition associated with elevated triglycerides is episodes of abdominal pain, physical fatigue, difficulty thinking, diarrhea, recurrent acute pancreatitis, eruptive cutaneous xanthomata, and hepatosplenomegaly or a combination thereof.
147. The method of any one or embodiments 135-138, wherein the method prevents or protects against progression of coronary heart disease (CHD).
148. The method of any one of embodiments 133-147, wherein administering of the oligomeric duplex or oligomeric agent of any one of embodiments 1-124, the population of any one of embodiments 125-126, or the antisense agent of embodiment 128 or embodiment 129, or the pharmaceutical composition of any one of embodiments 130-132 improves chylomicronemia, hypertriglyceridemia, abdominal pain, physical fatigue, difficulty thinking, diarrhea, acute pancreatitis, eruptive xanthomas, lipemia retinalis, or hepatosplenomegaly, or a combination of two or more of the foregoing in the subject.
149. The method of any one of embodiments 133-147, wherein administering of the oligomeric duplex or oligomeric agent of any one of embodiments 1-124, the population of any one of embodiments 125-126, or the antisense agent of embodiment 128 or embodiment 129, or the pharmaceutical composition of any one of embodiments 130-132 is parenteral.
150. The method of any one of embodiments 133-147, wherein administering of the oligomeric duplex or oligomeric agent of any one of embodiments 1-124, the population of any one of embodiments 125-126, or the antisense agent of embodiment 128 or embodiment 129, or the pharmaceutical composition of any one of embodiments 130-132 is subcutaneous.
151. The method of any one of embodiments 133-147, wherein administering of the oligomeric duplex or oligomeric agent of any one of embodiments 1-124, the population of any one of embodiments 125-126, or the antisense agent of embodiment 128 or embodiment 129, or the pharmaceutical composition of any one of embodiments 130-132 is co-administering with a second agent.
152. The method of embodiment 151, wherein administering of the oligomeric duplex or oligomeric agent of any one of embodiments 1-135, the population of any one of embodiments 136-137, or the antisense agent of embodiment 139 or embodiment 140, or the pharmaceutical composition of any one of embodiments 141-143 and the second agent are administered concomitantly.
153. Use of the oligomeric duplex or oligomeric agent of any one of embodiments 1-124, the population of any one of embodiments 125-126, or the antisense agent of embodiment 128 or embodiment 129, or the pharmaceutical composition of any one of embodiments 130-132 for treating or preventing a disease, disorder or condition associated with lipoprotein metabolism misregulation or postponing a symptom of a disease, disorder or condition associated with elevated triglycerides.
154. Use of the oligomeric duplex or oligomeric agent of any one of embodiments 1-124, the population of any one of embodiments 125-126, or the antisense agent of embodiment 128 or embodiment 129, or the pharmaceutical composition of any one of embodiments 130-132 in the manufacture of a medicament for treating or preventing a cardiovascular disease, disorder, condition, a metabolic disease, disorder, or condition, or an inflammatory disease disorder or condition.
155. The use of embodiment 153 or 154, wherein the disease, disorder or condition wherein the disease, disorder or condition is hypertriglyceridemia or atherosclerotic cardiovascular disease (ASCVD) or coronary artery disease (CAD).
156. The use of embodiment 155, wherein the ASCVD is ischemic vascular disease (IVD).
157. The use of embodiment 155, wherein the ASCVD is ischemic heart disease (IHD).
158. The use of embodiment 155 wherein the hypertriglyceridemia is genetic hypertriglyceridemia.
159. The use of embodiment 155, wherein the hypertriglyceridemia is familial chylomicronemia syndrome (FCS) or familial partial lipodystrophy (FPL).
160. The oligomeric duplex or oligomeric agent of any one of embodiments 1-124, the population of any one of embodiments 125-126, or the antisense agent of embodiment 128 or embodiment 129, or the pharmaceutical composition of any one of embodiments 130-132 for use in treating or preventing a disease, disorder or condition associated with lipoprotein metabolism misregulation or postponing a symptom of a disease, disorder or condition associated with elevated triglycerides.
161. The oligomeric duplex or oligomeric agent of any one of embodiments 1-124, the population of any one of embodiments 125-126, or the antisense agent of embodiment 128 or embodiment 129, or the pharmaceutical composition of any one of embodiments 130-132 for use in the manufacture of a medicament for treating or preventing a cardiovascular disease, disorder, condition, a metabolic disease, disorder, or condition, or an inflammatory disease disorder or condition.
162. The oligomeric duplex for use of embodiment 160 or 161, wherein the disease, disorder or condition wherein the disease, disorder or condition is hypertriglyceridemia or atherosclerotic cardiovascular disease (ASCVD) or coronary artery disease (CAD).
163. The oligomeric duplex for use of embodiment 162, wherein the ASCVD is ischemic vascular disease (IVD).
164. The oligomeric duplex for use of embodiment 162, wherein the ASCVD is ischemic heart disease (THD).
165. The oligomeric duplex for use of embodiment 162, wherein the hypertriglyceridemia is genetic hypertriglyceridemia.
166. The oligomeric duplex for use of embodiment 162, wherein the hypertriglyceridemia is familial chylomicronemia syndrome (FCS) or familial partial lipodystrophy (FPL).

### I. Oligonucleotides

In certain embodiments, provided herein are oligomeric duplexes and oligomeric duplex conjugates comprising a modified antisense oligonucleotide (e.g., an antisense oligomeric compound) complementary to APOC3 RNA and a modified sense oligonucleotide (e.g., a sense oligomeric compound) complementary to an antisense oligomeric compound. Modified antisense and/or sense oligonucleotides comprise at least one modified nucleoside (comprising a modified sugar moiety and/or a modified nucleobase) and/or at least one modified internucleoside linkage. Examples of certain modified nucleosides and modified internucleoside linkages suitable for use in modified antisense and/or sense oligonucleotides are described herein.

### A. Modified Nucleosides

Modified nucleosides comprise a modified sugar moiety or a modified nucleobase or both a modified sugar moiety and a modified nucleobase. In certain embodiments, modified nucleosides comprising the following modified sugar moieties and/or the following modified nucleobases may be incorporated into modified antisense and/or sense oligonucleotides.

### 1. Modified Sugar Moieties

In certain embodiments, a modified sugar moiety is a non-bicyclic modified sugar moiety. In certain embodiments, a modified sugar moiety is a bicyclic or tricyclic sugar moiety. In certain embodiments, modified a sugar moiety is a sugar surrogate. Sugar surrogates may comprise one or more substitutions corresponding to those of other types of modified sugar moieties. In certain embodiments, a modified sugar moiety is a modified ribosyl sugar moiety. In certain embodiments, a modified sugar moiety is a 2′-deoxyfuranosyl sugar moiety.

In certain embodiments, modified sugar moieties are non-bicyclic modified furanosyl sugar moieties comprising one or more substituent groups including, but not limited to, substituents at the 2′, 3′, 4′, and/or 5′ positions. In certain embodiments, the furanosyl sugar moiety is a ribosyl sugar moiety. In certain embodiments one or more non-bridging substituent of non-bicyclic modified sugar moieties is branched.

In certain embodiments, non-bicyclic modified sugar moieties comprise a substituent group at the 2′-position. Examples of substituent groups suitable for the 2′-position of modified sugar moieties include but are not limited to: 2′-F, 2′-OCH₃ (“OMe” or “O-methyl”), and 2′-O(CH₂)₂OCH₃ (“MOE” or “O-methoxyethyl”). In certain embodiments, 2′-substituent groups are selected from among: halo, allyl, amino, azido, SH, CN, OCN, CF₃, OCF₃, O—C₁-C₁₀ alkoxy, O—C₁-C₁₀ substituted alkoxy, O—C₁-C₁₀ alkyl, O—C₁-C₁₀ substituted alkyl, S-alkyl, N(Rₘ)-alkyl, O-alkenyl, S-alkenyl, N(Rₘ)-alkenyl, O-alkynyl, S-alkynyl, N(Rₘ)-alkynyl, O-alkylenyl-O-alkyl, alkynyl, alkaryl, aralkyl, O-alkaryl, O-aralkyl, O(CH₂)₂SCH₃, O(CH₂)₂ON(Rₘ)(Rₙ) or OCH₂C(═O)—N(Rₘ)(Rₙ), where each Rₘ and Rₙ is, independently, H, an amino protecting group, or substituted or unsubstituted C₁-C₁₀ alkyl, —O(CH₂)₂ON(CH₃)₂ (“DMAOE”), or 2′-O(CH₂)₂O(CH₂)₂N(CH₃)₂ (“DMAEOE”). Synthetic methods for some of these 2′-substituent groups can be found, e.g., in Cook et al., U.S. Pat. No. 6,531,584; Cook et al., U.S. Pat. No. 5,859,221; and Cook et al., U.S. Pat. No. 6,005,087. Certain embodiments of these 2′-substituent groups can be further substituted with one or more substituent groups independently selected from among: hydroxyl, amino, alkoxy, carboxy, benzyl, phenyl, nitro (NO₂), thiol, thioalkoxy, thioalkyl, halogen, alkyl, aryl, alkenyl and alkynyl.

In certain embodiments, a 2′-substituted non-bicyclic modified nucleoside comprises a sugar moiety comprising a non-bridging 2′-substituent group selected from: F, NH₂, N₃, OCF₃, OCH₃, O(CH₂)₃NH₂, CH₂CH═CH₂, OCH₂CH═CH₂, OCH₂CH₂OCH₃, O(CH₂)₂SCH₃, O(CH₂)₂ON(Rₘ)(Rₙ), O(CH₂)₂O(CH₂)₂N(CH₃)₂, and N-substituted acetamide (OCH₂C(═O)—N(Rₘ)(Rₙ)), where each Rₘ and Rₙ is, independently, H, an amino protecting group, or substituted or unsubstituted C₁-C₁₀ alkyl.

In certain embodiments, a 2′-substituted sugar moiety of a modified nucleoside comprises 2′-substituent group selected from: F, OCF₃, OCH₃, OCH₂CH₂OCH₃, O(CH₂)₂SCH₃, O(CH₂)₂ON(CH₃)₂, O(CH₂)₂O(CH₂)₂N(CH₃)₂, O(CH₂)₂ON(CH₃)₂ (“DMAOE”), O(CH₂)₂O(CH₂)₂N(CH₃)₂ (“DMAEOE”), and OCH₂C(═O)—N(H)CH₃ (“NMA”).

In certain embodiments, a 2′-substituted non-bicyclic modified nucleoside comprises a sugar moiety comprising a non-bridging 2′-substituent group selected from: F, OCF₃, OCH₃, OCH₂CH₂OCH₃, O(CH₂)₂SCH₃, O(CH₂)₂ON(CH₃)₂, O(CH₂)₂O(CH₂)₂N(CH₃)₂, and OCH₂C(═O)—N(H)CH₃ (“NMA”).

In certain embodiments, a 2′-substituted sugar moiety of a modified nucleoside comprises a 2′-substituent group selected from: F, OCH₃, and OCH₂CH₂OCH₃.

In certain embodiments, modified furanosyl sugar moieties and nucleosides incorporating such modified furanosyl sugar moieties are further defined by isomeric configuration. For example, a 2′-furanosyl sugar moiety (i.e., 2′-(H)OH furanosyl sugar moiety) may be in seven isomeric configurations other than the naturally occurring β-D-ribosyl configuration. Such modified sugar moieties are described in, e.g., WO2020/072991, incorporated by reference herein. A 2′-modified sugar moiety has an additional stereocenter at the 2′-position relative to a 2′-furanosyl sugar moiety; therefore, such sugar moieties have a total of sixteen possible isomeric configurations. Modified furanosyl sugar moieties described herein are in the β-D-ribosyl isomeric configuration unless otherwise specified.

In certain embodiments, non-bicyclic modified sugar moieties comprise a substituent group at the 4′-position. Examples of substituent groups suitable for the 4′-position of modified sugar moieties include, but are not limited to, alkoxy (e.g., methoxy), alkyl, and those described in Manoharan et al., WO 2015/106128.

In certain embodiments, non-bicyclic modified sugar moieties comprise a substituent group at the 3′-position. Examples of substituent groups suitable for the 3′-position of modified sugar moieties include, but are not limited to, alkoxy (e.g., methoxy), alkyl (e.g., methyl, ethyl).

In certain embodiments, non-bicyclic modified sugar moieties comprise a substituent group at the 5′-position. Examples of substituent groups suitable for the 5′-position of modified sugar moieties include, but are not limited to, vinyl, alkoxy (e.g., methoxy), and alkyl (e.g., methyl (R or S), ethyl).

In certain embodiments, non-bicyclic modified sugar moieties comprise more than one non-bridging sugar substituent, for example, 2′-F-5′-methyl sugar moieties, such as described in Migawa et al., US2010/0190837, or alternative 2′- and 5′-modified sugar moieties as described in Rajeev et al., US2013/0203836.

In naturally occurring nucleic acids, sugars are linked to one another 3′ to 5′. In certain embodiments, oligonucleotides include one or more nucleoside or sugar moiety linked at an alternative position, for example at the 2′ or inverted 5′ to 3′. For example, where the linkage is at the 2′ position, the 2′-substituent groups may instead be at the 3′-position. As used herein, “inverted nucleoside” means a nucleotide having a non-natural linkage, e.g., a 3′ to 3′ and/or 5′ to 5′ internucleoside linkage, as shown herein.

Certain modified sugar moieties comprise a substituent that bridges two atoms of the furanosyl ring to form a second ring, resulting in a bicyclic sugar moiety. In certain embodiments, the bicyclic sugar moiety comprises a bridge between the 4′ and the 2′ furanose ring atoms. Examples of such 4′ to 2′ bridging sugar substituents include, but are not limited to: 4′-CH₂-2′, 4′-(CH₂)₂-2′, 4′-(CH₂)₃-2′, 4′-CH₂—O-2′ (“LNA”), 4′-CH₂—S-2′, 4′-(CH₂)₂—O-2′ (“ENA”), 4′-CH(CH₃)—O-2′ (referred to as “constrained ethyl” or “cEt” when in the S configuration), 4′-CH₂—O—CH₂-2′, 4′-CH₂—N(R)-2′, 4′-CH(CH₂OCH₃)—O-2′ (“constrained MOE” or “cMOE”) and analogs thereof, 4′-C(CH₃)(CH₃)—O-2′ and analogs thereof, 4′-CH₂—N(OCH₃)-2′ and analogs thereof, 4′-CH₂—O—N(CH₃)-2′, 4′-CH₂—C(H)(CH₃)-2′, 4′-CH₂—C(═CH₂)-2′ and analogs thereof), 4′-C(RₐR_{b})—N(R)—O-2′, 4′-C(RₐR_{b})—O—N(R)-2′, 4′-CH₂—O—N(R)-2′, and 4′-CH₂—N(R)—O-2′, wherein each R, Rₐ, and R_{b} is, independently, H, a protecting group, or C₁-C₁₂ alkyl. Representative U.S. patents that teach the preparation of such bicyclic sugar moieties include, but are not limited to: Imanishi et al., U.S. Pat. No. 7,427,672; Swayze et al., U.S. Pat. No. 7,741,457, and Swayze et al., U.S. Pat. No. 8,022,193; Seth et al., U.S. Pat. No. 8,278,283; Prakash et al., U.S. Pat. No. 8,278,425; Seth et al., U.S. Pat. No. 8,278,426.

In certain embodiments, such 4′ to 2′ bridges independently comprise from 1 to 4 linked groups independently selected from: —[C(Rₐ)(R_{b})]ₙ—, —[C(Rₐ)(R_{b})]ₙ—O—, C(Rₐ)═C(R_{b})—, C(Rₐ)═N—, C(═NRₐ)—, —C(═O)—, —C(═S)—, —O—, —Si(Rₐ)₂—, —S(═O)ₓ—, and N(Rₐ)—; wherein: x is 0, 1, or 2; n is 1, 2, 3, or 4; each Rₐ and R_{b} is, independently, H, a protecting group, hydroxyl, C₁-C₁₂ alkyl, substituted C₁-C₁₂ alkyl, C₂-C₁₂ alkenyl, substituted C₂-C₁₂ alkenyl, C₂-C₁₂ alkynyl, substituted C₂-C₁₂ alkynyl, C₅-C₂₀ aryl, substituted C₅-C₂₀ aryl, heterocycle radical, substituted heterocycle radical, heteroaryl, substituted heteroaryl, C₅-C₇ alicyclic radical, substituted C₅-C₇ alicyclic radical, halogen, OJ¹, NJ¹J², SJ¹, N₃, COOJ¹, acyl (C(═O)—H), substituted acyl, CN, sulfonyl (S(═O)₂-J¹), or sulfoxyl (S(═O)-J¹); and each J¹ and J² is, independently, H, C₁-C₁₂ alkyl, substituted C₁-C₁₂ alkyl, C₂-C₁₂ alkenyl, substituted C₂-C₁₂ alkenyl, C₂-C₁₂ alkynyl, substituted C₂-C₁₂ alkynyl, C₅-C₂₀ aryl, substituted C₅-C₂₀ aryl, acyl (C(═O)—H), substituted acyl, a heterocycle radical, a substituted heterocycle radical, C₁-C₁₂ aminoalkyl, substituted C₁-C₁₂ aminoalkyl, or a protecting group.

Additional bicyclic sugar moieties are known in the art, see, for example: Wan, et al., J. Medicinal Chemistry, 2016, 59, 9645-9667; Wengel et al., U.S. Pat. No. 8,080,644; Ramasamy et al., U.S. Pat. No. 6,525,191; Seth et al., U.S. Pat. No. 7,547,684; and Seth et al., U.S. Pat. No. 7,666,854.

In certain embodiments, bicyclic sugar moieties and nucleosides incorporating such bicyclic sugar moieties are further defined by isomeric configuration. For example, an LNA nucleoside (described herein) may be in the α-L configuration or in the β-D configuration.

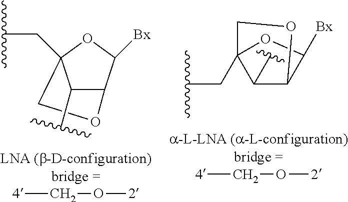

α-L-methyleneoxy (4′-CH₂—O-2′) or α-L-LNA bicyclic nucleosides have been incorporated into oligonucleotides that showed antisense activity (Frieden et al., Nucleic Acids Research, 2003, 21, 6365-6372). The addition of locked nucleic acids to siRNAs has been shown to increase siRNA stability in serum, and to reduce off-target effects (Elmen, J. et al., (2005) Nucleic Acids Research 33(1):439-447; Mook, O R. et al., (2007) Mol Canc Ther 6(3):833-843; Grunweller, A. et al., (2003) Nucleic Acids Research 31(12):3185-3193). Herein, general descriptions of bicyclic nucleosides include both isomeric configurations. When the positions of specific bicyclic nucleosides (e.g., LNA or cEt) are identified in exemplified embodiments herein, they are in the β-D configuration, unless otherwise specified.

In certain embodiments, modified sugar moieties comprise one or more non-bridging sugar substituent and one or more bridging sugar substituent (e.g., 5′-substituted and 4′-2′ bridged sugars).

In certain embodiments, modified sugar moieties are sugar surrogates. In certain such embodiments, the oxygen atom of the sugar moiety is replaced, e.g., with a sulfur, carbon or nitrogen atom. In certain such embodiments, such modified sugar moieties also comprise bridging and/or non-bridging substituents as described herein. For example, certain sugar surrogates comprise a 4′-sulfur atom and a substitution at the 2′-position and/or the 5′ position.

In certain embodiments, sugar surrogates comprise rings having other than 5 atoms. For example, in certain embodiments, a sugar surrogate comprises a six-membered tetrahydropyran (“THP”), where X is O—C(R₁R₂), p is 1, Z is C(G₁G₂), and m is 0. Such tetrahydropyrans may be further modified or substituted. Nucleosides comprising such modified tetrahydropyrans include but are not limited to hexitol nucleic acid (“HNA”), altritol nucleic acid (G₁=OH; G₂=H; “ANA”), and fluoro HNA:

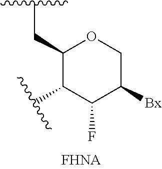

(G₁=F; G₂=H; “FHNA”, see e.g., Egli, M. et al. J. Am. Chem. Soc. 2011, 133(41), 16642-16649; Swayze et al., U.S. Pat. No. 8,088,904; and Swayze et al., U.S. Pat. No. 8,440,803); FHNA can also be referred to as a F-THP or 3′-fluoro tetrahydropyran or 3′-FHNA), each of which are incorporated herein by reference.

### Modified Nucleobases

In certain embodiments, modified oligonucleotides comprise one or more nucleoside comprising an unmodified nucleobase. In certain embodiments, modified oligonucleotides comprise one or more nucleoside comprising a modified nucleobase. In certain embodiments, modified oligonucleotides comprise one or more nucleoside that does not comprise a nucleobase, referred to as an abasic nucleoside. In certain embodiments, modified oligonucleotides contain no abasic nucleosides. In certain embodiments, modified oligonucleotides comprise one or more inosine nucleosides (i.e., nucleosides comprising a hypoxanthine nucleobase). An “unmodified nucleobase” is unmodified adenine (A), unmodified thymine (T), unmodified cytosine (C), unmodified uracil (U), or unmodified guanine (G). A modified nucleobase is a group of atoms other than unmodified A, T, C, U, or G capable of pairing with at least one other nucleobase. A 5-methylcytosine is an example of a modified nucleobase. A universal base is a modified nucleobase that can pair with any one of the five unmodified nucleobases.

Unless otherwise indicated, modified adenine has structure (I):

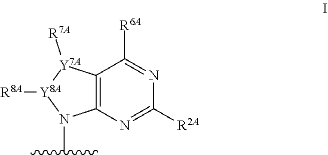

wherein: R^{2A} is H C₁-C₆ alkyl, substituted C₁-C₆ alkyl, C₁-C₆ thioalkyl, or substituted C₁-C₆ thioalkyl, C₁-C₆ alkyloxy, or substituted C₁-C₆ alkyloxy; R^{6A} is H, N(R^{a})(R^{b}), acetyl, formyl, or O-phenyl; Y^{7A} is N and R^{7A} is absent or is C₁-C₆ alkyl; or Y^{7A} is C and R^{7A} is selected from H, C₁-C₆ alkyl, or N(R^{a})(R^{b}); Y^{8A} is N and R^{8A} is absent, or Y^{8A} is C and R^{8A} is selected from H, a halogen, OH, C₁-C₆ alkyl, or substituted C₁-C₆ alkyl; R^{a} and R^{b} are independently selected from H, C₁-C₆ alkyl, substituted C₁-C₆ alkyl, C₁-C₆ alkenyl, substituted C₁-C₆ alkenyl, acetyl, formyl, or together form a 5-7-membered heterocycle; excluding where Y^{7A} is N; Y^{8A} is C, R^{8A} is H, R^{2A} is H, and R^{6A} is NH₂ (unmodified adenine).

Unless otherwise indicated, modified guanine has structure (II):

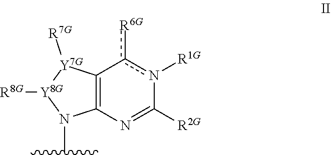

wherein: R^{2G} is N(R^{a})(R^{b}); R^{6G} is oxo and R^{1G} is H, or R^{6G} is selected from O—C₁-C₆ alkyl or S—C₁-C₆ alkyl and R^{1G} is absent; Y^{7G} is N and R^{7A} is absent or is C₁-C₆ alkyl; or Y^{7G} is C and R^{7G} is selected from H, C₁-C₆ alkyl, or N(R^{a})(R^{b}); Y^{8G} is N and R^{8G} is absent, or Y^{8G} is C and R^{8G} is selected from H, a halogen, OH, C₁-C₆ alkyl, or substituted C₁-C₆ alkyl; R^{a} and R are independently selected from H, C₁-C₆ alkyl, substituted C₁-C₆ alkyl, C₁-C₆ alkenyl, substituted C₁-C₆ alkenyl, acetyl, formyl, or together form a 5-7-membered heterocycle; excluding where Y^{7G} is N; Y^{8G} is C, R^{8G} is H, R^{2G} is NH₂, and R^{6G} is ═O (unmodified guanosine).

Unless otherwise indicated, modified thymine or modified uracil has structure (III):

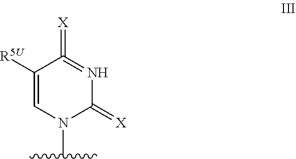

wherein: X is selected from O or S and R^{5U} is selected from H, OH, halogen, O—C₁-C₂₀ alkyl, O—C₁-C₁₂ substituted alkyl, C₁-C₁₂ alkyl, substituted C₁-C₁₂ alkyl, C₁-C₁₂ alkenyl, substituted C₁-C₁₂ alkenyl, C₁-C₁₂ alkynyl, substituted C₁-C₁₂ alkynyl; wherein if each X is O, R^{5U} is not H or CH₃ (unmodified uracil and unmodified thymine, respectively).

Unless otherwise indicated, modified cytosine has structure (IV):

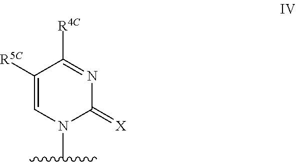

wherein: X is selected from O or S, R^{4C} is N(R^{a})(R^{b}); R^{5C} is selected from H, OH, halogen, O—C₁-C₁₂ alkyl, O—C₁-C₁₂ substituted alkyl, C₁-C₁₂ alkyl, substituted C₁-C₁₂ alkyl, C₁-C₁₂ alkenyl, substituted C₁-C₁₂ alkenyl; R^{a} and R are independently selected from H, C₁-C₆ alkyl, substituted C₁-C₆ alkyl, C₁-C₆ alkenyl, substituted C₁-C₆ alkenyl, C₁-C₁₂ alkynyl, substituted C₁-C₁₂ alkynyl; acetyl, formyl, or together form a 5-7-membered heterocycle; excluding where X is O, R^{4C} is NH₂ and RC is H (unmodified cytosine).

In certain embodiments, modified nucleobases of a modified oligonucleotide are selected from: 5-substituted pyrimidines, 6-azapyrimidines, alkyl or alkynyl substituted pyrimidines, alkyl substituted purines, and N-2, N-6 and O-6 substituted purines. In certain embodiments, modified nucleobases are selected from: 5-methylcytosine, 1-methylpsuedouridine, 2-aminopropyladenine, 5-hydroxymethyl cytosine, xanthine, hypoxanthine, 2-aminoadenine, 6-N-methylguanine, 6-N-methyladenine, 2-propyladenine, 2-thiouracil, 2-thiothymine and 2-thiocytosine, 5-propynyl (—C≡C—CH₃) uracil, 5-propynylcytosine, 6-azouracil, 6-azocytosine, 6-azothymine, 5-ribosyluracil (pseudouracil), 4-thiouracil, 8-halo, 8-amino, 8-thiol, 8-thioalkyl, 8-hydroxyl, 8-aza and other 8-substituted purines, 5-halo (particularly 5-bromo), 5-trifluoromethyl, 5-halouracil, and 5-halocytosine, 7-methylguanine, 7-methyladenine, 2-F-adenine, 2-aminoadenine, 7-deazaguanine, 7-deazaadenine, 3-deazaguanine, 3-deazaadenine, 6-N-benzoyladenine, 2-N-isobutyrylguanine, 4-N-benzoylcytosine, 4-N-benzoyluracil, 5-methyl 4-N-benzoylcytosine, 5-methyl 4-N-benzoyluracil, universal bases, hydrophobic bases, promiscuous bases, size-expanded bases, and fluorinated bases. Further modified nucleobases include tricyclic pyrimidines, such as 1,3-diazaphenoxazine-2-one, 1,3-diazaphenothiazine-2-one and 9-(2-aminoethoxy)-1,3-diazaphenoxazine-2-one (G-clamp). Modified nucleobases may also include those in which the purine or pyrimidine base is replaced with other heterocycles, for example 7-deaza-adenine, 7-deazaguanosine, 2-aminopyridine and 2-pyridone. Further nucleobases include those disclosed in Englisch et al., Angewandte Chemie, International Edition, 1991, 30, 613; Sanghvi, Y. S., Chapter 15, Antisense Research and Applications, Crooke, S. T. and Lebleu, B., Eds., CRC Press, 1993, 273-288; and those disclosed in Chapters 6 and 15, Antisense Drug Technology, Crooke S. T., Ed., CRC Press, 2008, 163-166 and 442-443.

Publications that teach the preparation of certain of the above noted modified nucleobases, as well as other modified nucleobases include without limitation, Rogers et al., U.S. Pat. No. 5,134,066; Benner et al., U.S. Pat. No. 5,432,272; Matteucci et al., U.S. Pat. No. 5,502,177; Froehler et al., U.S. Pat. No. 5,594,121; and Cook et al., U.S. Pat. No. 5,681,941.

In certain embodiments, each nucleobase of a modified oligonucleotide is selected from unmodified A, unmodified G, unmodified C, unmodified T, unmodified U, and ^{m}C.

In certain embodiments, there are no modified nucleobases in a modified oligonucleotide and each nucleobase of a modified oligonucleotide is selected from unmodified A, unmodified G, unmodified C, unmodified T, and unmodified U.

### 2. Modified Internucleoside Linkages

In certain embodiments, oligomeric agents provided herein comprise or consist of a modified oligonucleotide (e.g., an oligomeric compound) comprising at least one modified internucleoside linkage. The naturally occurring internucleoside linkage of RNA and DNA is a 3′ to 5′ phosphodiester linkage. In certain embodiments, nucleosides of modified oligonucleotides are linked together using one or more modified internucleoside linkages. The two main classes of internucleoside linkages are defined by the presence or absence of a phosphorus atom. Representative phosphorus-containing internucleoside linkages include but are not limited to phosphates, which contain a phosphodiester bond (“P=O”) (also referred to as unmodified or naturally occurring linkages), phosphotriesters, methylphosphonates, phosphoramidates, and phosphorothioates (“P=S”), and phosphorodithioates (“HS—P═S”). Representative non-phosphorus containing internucleoside linkages include but are not limited to methylenemethylimino (—CH₂—N(CH₃)—O—CH₂—), thiodiester, thionocarbamate (—O—C(═O)(NH)—S—); siloxane (—O—SiH₂—O—); and N,N′-dimethylhydrazine (—CH₂—N(CH₃)—N(CH₃)—). Modified internucleoside linkages, compared to naturally occurring phosphate linkages, can be used to alter, typically increase, nuclease resistance of the oligonucleotide.

In certain embodiments, a modified internucleoside linkage is any of those described in WO/2021/030778, incorporated by reference herein. In certain embodiments, a modified internucleoside linkage comprises the formula:

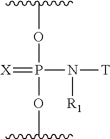

wherein independently for each internucleoside linkage of the modified oligonucleotide:
X is selected from O or S;
R₁ is selected from H, C₁-C₆ alkyl, and substituted C₁-C₆ alkyl; and
T is selected from SO₂R₂, C(═O)R₃, and P(═O)R₁R₅, wherein:
R₂ is selected from an aryl, a substituted aryl, a heterocycle, a substituted heterocycle, an aromatic heterocycle, a substituted aromatic heterocycle, a diazole, a substituted diazole, a C₁-C₆ alkoxy, C₁-C₆ alkyl, C₁-C₆ alkenyl, C₁-C₆ alkynyl, substituted C₁-C₆ alkyl, substituted C₁-C₆ alkenyl, substituted C₁-C₆ alkynyl, and a conjugate group;
R₃ is selected from an aryl, a substituted aryl, CH₃, N(CH₃)₂, OCH₃ and a conjugate group;
R₄ is selected from OCH₃, OH, C₁-C₆ alkyl, substituted C₁-C₆ alkyl and a conjugate group; and
R₅ is selected from OCH₃, OH, C₁-C₆ alkyl, and substituted C₁-C₆ alkyl.

In certain embodiments, a modified oligonucleotide comprises a mesyl phosphoramidate linkage having a formula:

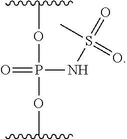 [SMILES: *OP(=O)(NS(C)(=O)=O)O*]

Certain internucleoside linkages having reduced charge (referred to as “neutral internucleoside linkages”) have been described. Such neutral internucleoside linkages include, without limitation, phosphotriesters, methylphosphonates, MMI (3′-CH₂—N(CH₃)—O-5′), amide-3 (3′-CH₂—C(═O)—N(H)-5′), amide-4 (3′-CH₂—N(H)—C(═O)-5′), formacetal (3′-O—CH₂—O-5′), methoxypropyl (MOP), and thioformacetal (3′-S—CH₂—O-5′). Further neutral internucleoside linkages include nonionic linkages comprising siloxane (dialkylsiloxane), carboxylate ester, carboxamide, sulfide, sulfonate ester and amides (See for example: Carbohydrate Modifications in Antisense Research; Y. S. Sanghvi and P. D. Cook, Eds., ACS Symposium Series 580; Chapters 3 and 4, 40-65). Further neutral internucleoside linkages include nonionic linkages comprising mixed N, O, S and CH₂ component parts.

In certain embodiments, modified oligonucleotides comprise one or more inverted nucleoside, as shown below:

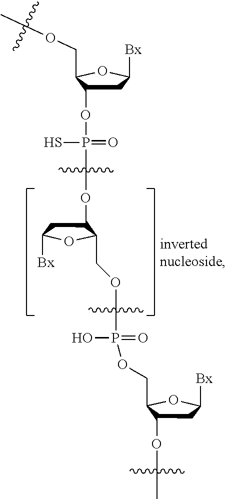

wherein each Bx independently represents any nucleobase.

In certain embodiments, an inverted nucleoside is terminal (i.e., the last nucleoside on one end of an oligonucleotide) and so only one internucleoside linkage depicted above will be present. In certain embodiments, additional features (e.g., a conjugate group) are attached to the inverted nucleoside. Such terminal inverted nucleosides can be attached to either or both ends of an oligonucleotide.

In certain embodiments, inverted nucleosides lack a nucleobase and are referred to herein as inverted sugar moieties. In certain embodiments, an inverted sugar moiety is terminal (i.e., attached to the last nucleoside on one end of an oligonucleotide) and so only one internucleoside linkage above will be present. In certain such embodiments, additional features (e.g., a conjugate group) are attached to the inverted sugar moiety. A terminal inverted sugar moiety can be attached to either or both ends of an oligonucleotide.

In certain embodiments, nucleosides are linked 2′ to 5′ rather than the standard 3′ to 5′ linkage. Such a linkage is illustrated below.

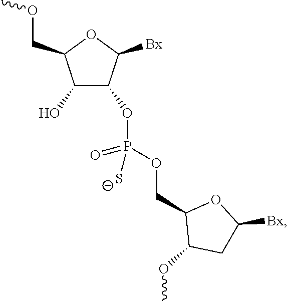

wherein each Bx represents any nucleobase.

In certain embodiments, internucleoside linkages have at least one chiral center. In such embodiments, a chiral atom can be prepared as a racemic mixture, or as separate enantiomers. Representative internucleoside linkages having a chiral center include but are not limited to alkylphosphonates, mesyl phosphoramidates, and phosphorothioates.

The phosphorothioate internucleoside linkage comprises a chiral center. In certain embodiments, modified oligonucleotides comprising (Rp) and/or (Sp) phosphorothioates comprise one or more of the following formulas, respectively, wherein “B” indicates a nucleobase:

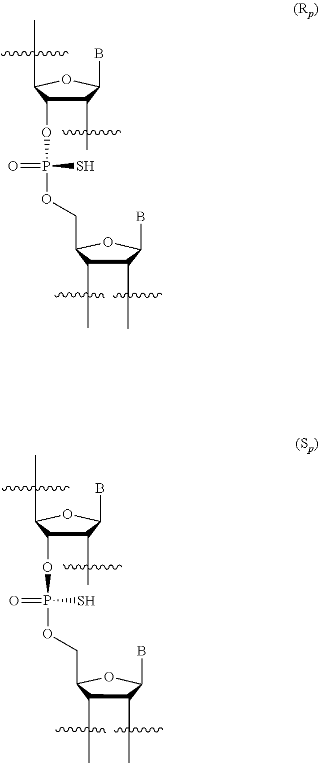

Modified oligonucleotides comprising internucleoside linkages having a chiral center can be prepared as populations of modified oligonucleotides comprising stereorandom internucleoside linkages, or as populations of modified oligonucleotides comprising linkages containing chiral centers in particular stereochemical configurations. In certain embodiments, populations of modified oligonucleotides comprise one or more phosphorothioate internucleoside linkages wherein all of the phosphorothioate internucleoside linkages are stereorandom. Such modified oligonucleotides can be generated using synthetic methods that result in random selection of the stereochemical configuration of each phosphorothioate linkage. Nonetheless, each individual phosphorothioate of each individual oligonucleotide molecule has a defined stereoconfiguration. In certain embodiments, populations of modified oligonucleotides are enriched for modified oligonucleotides comprising one or more particular phosphorothioate internucleoside linkages in a particular, independently selected stereochemical configuration. In certain embodiments, the particular configuration of the particular phosphorothioate linkage is present in at least 65% of the molecules in the population. In certain embodiments, the particular configuration of the particular phosphorothioate linkage is present in at least 70% of the molecules in the population. In certain embodiments, the particular configuration of the particular phosphorothioate linkage is present in at least 80% of the molecules in the population. In certain embodiments, the particular configuration of the particular phosphorothioate linkage is present in at least 90% of the molecules in the population. In certain embodiments, the particular configuration of the particular phosphorothioate linkage is present in at least 99% of the molecules in the population. Such chirally enriched populations of modified oligonucleotides can be generated using synthetic methods known in the art, e.g., methods described in Oka et al., JACS 125, 8307 (2003), Wan et al. Nuc. Acid. Res. 42, 13456 (2014), and WO 2017/015555. As used herein, “chirally enriched” in reference to a population means a plurality of molecules of identical molecular formula, wherein the number or percentage of molecules within the population that contain a particular stereochemical configuration at a particular chiral center is greater than the number or percentage of molecules expected to contain the same particular stereochemical configuration at the same particular chiral center within the population if the particular chiral center were stereorandom as defined herein. Chirally enriched populations of molecules having multiple chiral centers within each molecule may contain one or more stereorandom chiral centers. In certain embodiments, the molecules are modified oligonucleotides. In certain embodiments, the molecules are oligomeric agents comprising modified oligonucleotide (e.g., oligomeric compound). In certain embodiments, the chiral center is at the phosphorous atom of a phosphorothioate internucleoside linkage. In certain embodiments, a population of modified oligonucleotides is enriched for modified oligonucleotides having at least one indicated phosphorothioate in the (Sp) configuration. In certain embodiments, a population of modified oligonucleotides is enriched for modified oligonucleotides having at least one phosphorothioate in the (Rp) configuration. Unless otherwise indicated, chiral internucleoside linkages of modified oligonucleotides described herein can be stereorandom or in a particular stereochemical configuration. In certain embodiments, modified oligonucleotides of a chirally enriched population are enriched for R-D ribosyl sugar moieties, and all of the phosphorothioate internucleoside linkages are stereorandom. In certain embodiments, modified oligonucleotides of a chirally enriched population are enriched for R-D ribosyl sugar moieties, at least one particular phosphorothioate internucleoside linkage in a particular stereochemical configuration is enriched. In certain embodiments, modified oligonucleotides of a chirally enriched population are enriched for β-D ribosyl sugar moieties, and all of the phosphorothioate internucleoside linkages are stereorandom. In certain embodiments, modified oligonucleotides of a chirally enriched population are enriched for both β-D ribosyl sugar moieties and at least one, particular phosphorothioate internucleoside linkage in a particular stereochemical configuration is enriched.

### B. Motifs

In certain embodiments, modified oligonucleotides comprise one or more modified nucleosides comprising a modified sugar moiety. In certain embodiments, modified oligonucleotides comprise one or more modified nucleosides comprising a modified nucleobase. In certain embodiments, modified oligonucleotides comprise one or more modified internucleoside linkage. In certain such embodiments, the modified, unmodified, and differently modified sugar moieties, nucleobases, and/or internucleoside linkages of a modified oligonucleotide define a pattern or motif. In certain embodiments, the patterns of sugar moieties, nucleobases, and internucleoside linkages are each independent of one another. Thus, a modified oligonucleotide may be described by its sugar motif, nucleobase motif and/or internucleoside linkage motif (as used herein, nucleobase motif describes the modifications to the nucleobases independent of the nucleobase sequence).

### 1. Sugar Motifs

In certain embodiments, oligonucleotides comprise one or more type of modified sugar and/or unmodified sugar moiety arranged along the oligonucleotide or region thereof in a defined pattern or sugar motif. In certain instances, such sugar motifs include but are not limited to any of the sugar modifications discussed herein. In certain embodiments, the sugar moiety of at least one nucleoside of an antisense oligomeric compound is a modified sugar moiety. In certain embodiments, the sugar moiety of at least one nucleoside of a sense oligomeric compound is a modified sugar moiety.

In certain embodiments, modified oligonucleotides comprise or consist of a region having a fully modified sugar motif. In such embodiments, each nucleoside of the fully modified region of the modified oligonucleotide comprises a modified sugar moiety. In certain embodiments, each nucleoside of the entire modified oligonucleotide comprises a modified sugar moiety. In certain embodiments, modified oligonucleotides comprise or consist of a region having a fully modified sugar motif, wherein each nucleoside within the fully modified region comprises the same modified sugar moiety, referred to herein as a uniformly modified sugar motif. In certain embodiments, a fully modified oligonucleotide is a uniformly modified oligonucleotide. In certain embodiments, each nucleoside of a uniformly modified oligonucleotide comprises the same 2′-modification. In certain embodiments, every other nucleoside of a uniformly modified oligonucleotide comprises the same 2′-modification, resulting in an alternating 2′-modifications. In certain embodiments, neighboring nucleosides comprise different 2′-modification, and every other nucleoside of a uniformly modified oligonucleotide comprises the same 2′-modification, resulting in a uniform, alternating 2′-modification motif.

In certain embodiments, at least one nucleoside of a modified oligonucleotide comprises a 2′-OMe sugar moiety. In certain embodiments, at least 8 nucleosides comprise 2′-OMe sugar moieties. In certain embodiments, at least 10 nucleosides comprise 2′-OMe sugar moieties. In certain embodiments, at least 12 nucleosides comprise 2′-OMe sugar moieties. In certain embodiments, at least 13 nucleosides comprise 2′-OMe sugar moieties. In certain embodiments, at least 14 nucleosides comprise 2′-OMe sugar moieties. In certain embodiments, at least 15 nucleosides comprise 2′-OMe sugar moieties. In certain embodiments, at least 16 nucleosides comprise 2′-OMe sugar moieties. In certain embodiments, at least 17 nucleosides comprise 2′-OMe sugar moieties. In certain such embodiments, at least 18 nucleosides comprise 2′-OMe sugar moieties. In certain such embodiments, at least 20 nucleosides comprise 2′-OMe sugar moieties.

In certain embodiments, at least one nucleoside of a modified oligonucleotide comprises a 2′-F sugar moiety (i.e., a 2′-F modified nucleoside). In certain embodiments, at least 2 nucleosides comprise 2′-F sugar moieties. In certain embodiments, at least 3 nucleosides comprise 2′-F sugar moieties. In certain embodiments, 4 nucleosides comprise a 2′-F sugar moiety. In certain embodiments, at least one, but not more than four nucleosides comprise a 2′-F sugar moiety. In certain embodiments, 1 or 2 nucleosides comprise 2′-F sugar moieties. In certain embodiments, 1-3 nucleosides comprise 2′-F sugar moieties. In certain embodiments, only one nucleoside comprises a 2′-F sugar moiety. In certain embodiments, an antisense oligomeric compound comprises 2 to 4 non-contiguous 2′-F modified nucleosides. In certain embodiments, 4 nucleosides of an antisense oligomeric compound are 2′-F modified nucleosides and none of those 2′-F modified nucleosides are contiguous. In certain embodiments, 1, 2, 3, or 4 nucleosides of an antisense oligomeric compound are 2′-F modified nucleosides and each of those 2′-F modified nucleosides are non-contiguous. In certain such embodiments at least fifteen of the remainder of the nucleosides are 2′-OMe modified nucleosides. In certain embodiments, one nucleoside of an antisense oligomeric compound is a 2′-F modified nucleoside and at least fifteen of the remainder of the nucleosides are 2′-OMe modified nucleosides.

In certain embodiments, at least one nucleoside of a modified oligonucleotide comprises a 2′-deoxyribosyl sugar moiety that has no additional modifications. In certain embodiments, at least one nucleoside comprises a 2′-deoxyribosyl sugar moiety. In certain embodiments, at least 2 nucleosides comprise a 2′-deoxyribosyl sugar moiety. In certain embodiments, at least 3 nucleosides comprise a 2′-deoxyribosyl sugar moiety. In certain embodiments, at least 4 nucleosides comprise a 2′-deoxyribosyl sugar moiety. In certain embodiments, one nucleoside comprises a 2′-deoxyribosyl sugar moiety. In certain embodiments, 1 or 3 nucleosides comprise a 2′-deoxyribosyl sugar moiety. In certain embodiments, 1-3 nucleosides comprise a 2′-deoxyribosyl sugar moiety. In certain embodiments, three nucleosides comprise a 2′-deoxyribosyl sugar moiety. In certain embodiments, 1, 2, 3, or 4 nucleosides of an antisense oligomeric compound are a 2′-deoxyribosyl sugar modified nucleoside and each 2′-deoxyribosyl modified nucleoside is non-contiguous. In certain embodiments, 1, or 3 nucleosides of an antisense oligomeric compound are a 2′-deoxyribosyl sugar modified nucleoside and each 2′-deoxyribosyl modified nucleoside is non-contiguous. In certain embodiments, no nucleosides of a sense oligomeric compound are a 2′-deoxyribosyl sugar modified nucleoside. In certain embodiments, three nucleosides of an antisense oligomeric compound are 2′-deoxyribosyl sugar modified nucleosides and no nucleoside of a sense oligomeric compound is a 2′-deoxyribosyl modified nucleoside. In certain embodiments, one nucleosides of an antisense oligomeric compound are 2′-deoxyribosyl sugar modified nucleosides and no nucleoside of a sense oligomeric compound is a 2′-deoxyribosyl modified nucleoside.

In certain embodiments, a sugar moiety of an antisense oligomeric compound is modified, wherein the modified sugar modifications and/or sugar surrogate is selected from 2′-F, 2′-MOE, 2′-OMe, and 2′-deoxyribosyl. In certain embodiments, a sugar motif (from 5′ to 3′) of the antisense oligomeric compound is selected from efyyyfyyyyyyyfyfyyyyyyy, efyyydyyyyyyyfyfyyyyyyy, efyyydyyyyyyydydyyyyyyy, efyyyyyyyyyyyfYyyyyyyyy, efyyyfyyeyyyyfyfyyyyyyy, efyyyfyyeeyyyfyfyyyyyyy, efyyydyyyyyyyfyfyyyyyee, efyyydyyyyyyydydyyyyyee, efyyydyyeyyyyfyfyyyyyee, efyyydyyeyyyydydyyyyyee, efyyydyyeeyyydydyyyyyee, efyyydyyeeyyyfyfyyyyyee, efyyyfyyyyyyyfyfyyyee, efyyydyyyyyyyfyfyyyee, and efyyydyyyyyyydydyyyee, wherein each ‘y’ represents a 2′-OMe sugar moiety, each ‘f’ represents a 2′-F sugar moiety, each ‘d’ represents a 2′-β-D-deoxyribosyl sugar moiety, and each ‘e’ represents a 2′-MOE sugar moiety. In certain embodiments, a sugar moiety of a sense oligomeric compound is modified, wherein the modified sugar moiety is selected from 2′-F, 2′-MOE, and 2′-OMe. In certain embodiments, a sugar motif (from 5′ to 3′) of a sense oligomeric compound is selected from among:

yyyyyyfyfffyyyyyyyyyy,
yyyyyyyyyffyyyyyyyyyy,
yyyyyyyfyfyfyyyyyyyyy,
eeyyyyyyyffyyyyyyyyee,
eeyyyyyyffyyyyyyyyyee,
yyyyyyfyfffyyyyyyyy,
yyyyyyfyfyyyyyyyyyy,
yyyyyyyfyfyyyyyyyyy,
yyyyyyyyfyfyyyyyyyy,
eeyyyyyyyffyyyyyyee,
eeyyyyyyffyyyyyyyee,
and
dyyyyyyfyfffyyyyyyyyyy,

wherein each ‘y’ represents a 2′-OMe sugar moiety, each ‘f’ represents a 2′-F sugar moiety, and each ‘e’ represents a 2′-MOE sugar moiety.

### 2. Nucleobase Motifs

In certain embodiments, oligonucleotides comprise modified and/or unmodified nucleobases arranged along the oligonucleotide or region thereof in a defined pattern or motif. In certain embodiments, at least one nucleobase is modified. In certain embodiments, none of the nucleobases are modified. In certain embodiments, at least one purine and/or at least pyrimidine is modified. In certain embodiments, at least one adenine is modified. In certain embodiments, at least one guanine is modified. In certain embodiments, at least one thymine is modified. In certain embodiments, at least one uracil is modified. In certain embodiments, at least one cytosine is modified. In certain embodiments, at least one of the cytosine nucleobases in a modified oligonucleotide is 5-methyl cytosine. In certain embodiments, all of the cytosine nucleobases are 5-methyl cytosines and all of the other nucleobases of the modified oligonucleotide are unmodified nucleobases. In certain embodiments, one or two of the cytosine nucleobases are 5-methylcytosines and all of the other nucleobases of the modified oligonucleotide are unmodified nucleobases. In certain embodiments, each nucleobase is selected from 5-methylcytosine, unmodified cytosine, unmodified thymine, unmodified uracil, unmodified adenine, and unmodified guanine. In certain embodiments, each nucleobase is selected from 5-methylcytosine, unmodified cytosine, unmodified thymine, unmodified adenine, and unmodified guanine. In certain embodiments, each nucleobase is selected from unmodified cytosine, unmodified thymine, unmodified uracil, unmodified adenine, and unmodified guanine. In certain embodiments, each nucleobase is selected from unmodified cytosine, unmodified thymine, unmodified adenine, and unmodified guanine.

### 3. Internucleoside Linkage Motifs

In certain embodiments, oligonucleotides comprise modified and unmodified internucleoside linkages arranged along the oligonucleotide or region thereof in a defined pattern or motif. In certain embodiments, each internucleoside linkage is a phosphodiester internucleoside linkage (P=O). In certain embodiments, each internucleoside linkage of a modified oligonucleotide is a phosphorothioate internucleoside linkage (P═S). In certain embodiments, each internucleoside linkage of a modified oligonucleotide is independently selected from a phosphorothioate internucleoside linkage, and phosphodiester internucleoside linkage. In certain embodiments, each internucleoside linkage of a modified oligonucleotide is independently selected from a phosphorothioate internucleoside linkage and a phosphodiester internucleoside linkage. In certain embodiments, each phosphorothioate internucleoside linkage is independently selected from a stereorandom phosphorothioate, a (Sp) phosphorothioate, and a (Rp) phosphorothioate.

In certain embodiments, at least one internucleoside linkage of the antisense oligomeric compound is a modified internucleoside linkage. In certain embodiments, the 5′-most internucleoside linkage (i.e., linking the first nucleoside from the 5′-end to the second nucleoside from the 5′-end) is modified. In certain embodiments, the two 5′-most internucleoside linkages are modified. In certain embodiments, the first one or 2 internucleoside linkages from the 3′-end are modified. In certain embodiments, the modified internucleoside linkage is a phosphorothioate internucleoside linkage.

In certain embodiments, the remaining internucleoside linkages are all unmodified phosphodiester internucleoside linkages. In certain embodiments an antisense oligomeric compound has an internucleoside linkage motif (from 5′ to 3′) of: ssooooooooooooooooooss, wherein each ‘o’ represents a phosphodiester internucleoside linkage and each ‘s’ represents a phosphorothioate internucleoside linkage. In certain embodiments an antisense oligomeric compound has an internucleoside linkage motif (from 5′ to 3′) of: ssooosooooooooooooooss, wherein each ‘o’ represents a phosphodiester internucleoside linkage and each ‘s’ represents a phosphorothioate internucleoside linkage. In certain embodiments an antisense oligomeric compound has an internucleoside linkage motif (from 5′ to 3′) of: ssooosooooooososooooss, wherein each ‘o’ represents a phosphodiester internucleoside linkage and each ‘s’ represents a phosphorothioate internucleoside linkage. In certain embodiments an antisense oligomeric compound has an internucleoside linkage motif (from 5′ to 3′) of: ssooooooooooooooooss, wherein each ‘o’ represents a phosphodiester internucleoside linkage and each ‘s’ represents a phosphorothioate internucleoside linkage. In certain embodiments an antisense oligomeric compound has an internucleoside linkage motif (from 5′ to 3′) of: ssooosooooooooooooss, wherein each ‘o’ represents a phosphodiester internucleoside linkage and each ‘s’ represents a phosphorothioate internucleoside linkage. In certain embodiments an antisense oligomeric compound has an internucleoside linkage motif (from 5′ to 3′) of: ssooosooooooososooss, wherein each ‘o’ represents a phosphodiester internucleoside linkage and each ‘s’ represents a phosphorothioate internucleoside linkage.

In certain embodiments, at least one internucleoside linkage of the sense oligomeric compound is a modified internucleoside linkage. In certain embodiments, the 5′-most internucleoside linkage (i.e., linking the first nucleoside from the 5′-end to the second nucleoside from the 5′-end) is modified. In certain embodiments, the two 5′-most internucleoside linkages are modified. In certain embodiments, the first one or 2 internucleoside linkages from the 3′-end are modified. In certain embodiments, the modified internucleoside linkage is a phosphorothioate internucleoside linkage. In certain embodiments, the remaining internucleoside linkages are all unmodified phosphodiester linkages. In certain embodiments a sense oligomeric compound has an internucleoside linkage motif (from 5′ to 3′) of: ssooooooosooooooooss, wherein each ‘o’ represents a phosphodiester internucleoside linkage and each ‘s’ represents a phosphorothioate internucleoside linkage. In certain embodiments a sense oligomeric compound has an internucleoside linkage motif (from 5′ to 3′) of: ssoooooosoooooooooss, wherein each ‘o’ represents a phosphodiester internucleoside linkage and each ‘s’ represents a phosphorothioate internucleoside linkage. In certain embodiments a sense oligomeric compound has an internucleoside linkage motif (from 5′ to 3′) of: ssooooooooooooooss, wherein each ‘o’ represents a phosphodiester internucleoside linkage and each ‘s’ represents a phosphorothioate internucleoside linkage. In certain embodiments a sense oligomeric compound has an internucleoside linkage motif (from 5′ to 3′) of: ssooooooosooooooss, wherein each ‘o’ represents a phosphodiester internucleoside linkage and each ‘s’ represents a phosphorothioate internucleoside linkage. In certain embodiments a sense oligomeric compound has an internucleoside linkage motif (from 5′ to 3′) of: ssoooooosoooooooss, wherein each ‘o’ represents a phosphodiester internucleoside linkage and each ‘s’ represents a phosphorothioate internucleoside linkage. In certain embodiments a sense oligomeric compound has an internucleoside linkage motif (from 5′ to 3′) of: ossooooooooooooooooss, wherein each ‘o’ represents a phosphodiester internucleoside linkage and each ‘s’ represents a phosphorothioate internucleoside linkage.

### C. Lengths

It is possible to increase or decrease the length of an oligonucleotide without eliminating activity. For example, in Woolf et al. (Proc. Natl. Acad. Sci. USA 89:7305-7309, 1992), a series of oligonucleotides 13-25 nucleobases in length were tested for their ability to induce cleavage of a target RNA in an oocyte injection model. Oligonucleotides 25 nucleobases in length with 8 or 11 mismatch bases near the ends of the oligonucleotides were able to direct specific cleavage of the target RNA, albeit to a lesser extent than the oligonucleotides that contained no mismatches. Similarly, target specific cleavage was achieved using 13 nucleobase oligonucleotides, including those with 1 or 3 mismatches.

In certain embodiments, oligonucleotides (including modified oligonucleotides) can have any of a variety of ranges of lengths. In certain embodiments, oligonucleotides consist of X to Y linked nucleosides, where X represents the fewest number of nucleosides in the range and Y represents the largest number nucleosides in the range. In certain such embodiments, X and Y are each independently selected from 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, and 50; provided that X≤Y. For example, in certain embodiments, oligonucleotides consist of 12 to 13, 12 to 14, 12 to 15, 12 to 16, 12 to 17, 12 to 18, 12 to 19, 12 to 20, 12 to 21, 12 to 22, 12 to 23, 12 to 24, 12 to 25, 12 to 26, 12 to 27, 12 to 28, 12 to 29, 12 to 30, 13 to 14, 13 to 15, 13 to 16, 13 to 17, 13 to 18, 13 to 19, 13 to 20, 13 to 21, 13 to 22, 13 to 23, 13 to 24, 13 to 25, 13 to 26, 13 to 27, 13 to 28, 13 to 29, 13 to 30, 14 to 15, 14 to 16, 14 to 17, 14 to 18, 14 to 19, 14 to 20, 14 to 21, 14 to 22, 14 to 23, 14 to 24, 14 to 25, 14 to 26, 14 to 27, 14 to 28, 14 to 29, 14 to 30, 15 to 16, 15 to 17, 15 to 18, 15 to 19, 15 to 20, 15 to 21, 15 to 22, 15 to 23, 15 to 24, 15 to 25, 15 to 26, 15 to 27, 15 to 28, 15 to 29, 15 to 30, 16 to 17, 16 to 18, 16 to 19, 16 to 20, 16 to 21, 16 to 22, 16 to 23, 16 to 24, 16 to 25, 16 to 26, 16 to 27, 16 to 28, 16 to 29, 16 to 30, 17 to 18, 17 to 19, 17 to 20, 17 to 21, 17 to 22, 17 to 23, 17 to 24, 17 to 25, 17 to 26, 17 to 27, 17 to 28, 17 to 29, 17 to 30, 18 to 19, 18 to 20, 18 to 21, 18 to 22, 18 to 23, 18 to 24, 18 to 25, 18 to 26, 18 to 27, 18 to 28, 18 to 29, 18 to 30, 19 to 20, 19 to 21, 19 to 22, 19 to 23, 19 to 24, 19 to 25, 19 to 26, 19 to 27, 19 to 28, 19 to 29, 19 to 30, 20 to 21, 20 to 22, 20 to 23, 20 to 24, 20 to 25, 20 to 26, 20 to 27, 20 to 28, 20 to 29, 20 to 30, 21 to 22, 21 to 23, 21 to 24, 21 to 25, 21 to 26, 21 to 27, 21 to 28, 21 to 29, 21 to 30, 22 to 23, 22 to 24, 22 to 25, 22 to 26, 22 to 27, 22 to 28, 22 to 29, 22 to 30, 23 to 24, 23 to 25, 23 to 26, 23 to 27, 23 to 28, 23 to 29, 23 to 30, 24 to 25, 24 to 26, 24 to 27, 24 to 28, 24 to 29, 24 to 30, 25 to 26, 25 to 27, 25 to 28, 25 to 29, 25 to 30, 26 to 27, 26 to 28, 26 to 29, 26 to 30, 27 to 28, 27 to 29, 27 to 30, 28 to 29, 28 to 30, or 29 to 30 linked nucleosides.

In certain embodiments, modified oligonucleotides (including antisense oligomeric compounds) comprise 16 linked nucleosides having no more than 1 to 3 mismatches to a target sequence. In certain embodiments, modified oligonucleotides (including antisense oligomeric compounds) comprise 17 linked nucleosides having no more than 1 to 3 mismatches to a target sequence. In certain embodiments, modified oligonucleotides (including antisense oligomeric compounds) comprise 18 linked nucleosides having no more than 1 to 3 mismatches to a target sequence. In certain embodiments, modified oligonucleotides (including antisense oligomeric compounds) comprise 19 linked nucleosides having no more than 1 to 3 mismatches to a target sequence. In certain embodiments, modified oligonucleotides (including antisense oligomeric compounds) comprise 20 linked nucleosides having no more than 1 to 3 mismatches to a target sequence. In certain embodiments, modified oligonucleotides (including antisense oligomeric compounds) comprise 21 linked nucleosides having no more than 1 to 3 mismatches to a target sequence. In certain embodiments, modified oligonucleotides (including antisense oligomeric compounds) comprise 22 linked nucleosides having no more than 1 to 3 mismatches to a target sequence. In certain embodiments, modified oligonucleotides (including antisense oligomeric compounds) comprise 23 linked nucleosides having no more than 1 to 3 mismatches to a target sequence.

In certain embodiments, modified oligonucleotides (including antisense oligomeric compounds) consist of 16 linked nucleosides. In certain embodiments, modified oligonucleotides (including antisense oligomeric compounds) consist of 17 linked nucleosides. In certain embodiments, modified oligonucleotides (including antisense oligomeric compounds) consist of 18 linked nucleosides. In certain embodiments, modified oligonucleotides (including antisense oligomeric compounds) consist of 19 linked nucleosides. In certain embodiments, modified oligonucleotides (including antisense oligomeric compounds) consist of 20 linked nucleosides. In certain embodiments, modified oligonucleotides (including antisense oligomeric compounds) consist of 21 linked nucleosides. In certain embodiments, modified oligonucleotides (including antisense oligomeric compounds) consist of 22 linked nucleosides. In certain embodiments, modified oligonucleotides (including antisense oligomeric compounds) consist of 23 linked nucleosides.

In certain embodiments, antisense oligomeric compounds consist of 12-30 linked nucleosides. In certain embodiments, antisense oligomeric compounds consist of 17-25 linked nucleosides. In certain embodiments, antisense oligomeric compounds consist of 17-23 linked nucleosides. In certain embodiments, antisense oligomeric compounds consist of 17-21 linked nucleosides. In certain embodiments, antisense oligomeric compounds consist of 18-30 linked nucleosides. In certain embodiments, antisense oligomeric compounds consist of 20-30 linked nucleosides. In certain embodiments, antisense oligomeric compounds consist of 21-30 linked nucleosides. In certain embodiments, antisense oligomeric compounds consist of 23-30 linked nucleosides. In certain embodiments, antisense oligomeric compounds consist of 18-25 linked nucleosides. In certain embodiments, antisense oligomeric compounds consist of 20-22 linked nucleosides. In certain embodiments, antisense oligomeric compounds consist of 21-23 linked nucleosides. In certain embodiments, antisense oligomeric compounds consist of 23-24 linked nucleosides. In certain embodiments, antisense oligomeric compounds consist of 20 linked nucleosides. In certain embodiments, antisense oligomeric compounds consist of 21 linked nucleosides. In certain embodiments, antisense oligomeric compounds consist of 22 linked nucleosides. In certain embodiments, antisense oligomeric compounds consist of 23 linked nucleosides.

In certain embodiments, sense oligomeric compounds consist of 12-30 linked nucleosides. In certain embodiments, sense oligomeric compounds consist of 16-25 linked nucleosides. In certain embodiments, sense oligomeric compounds consist of 16-23 linked nucleosides. In certain embodiments, sense oligomeric compounds consist of 16-21 linked nucleosides. In certain embodiments, sense oligomeric compounds consist of 16-30 linked nucleosides. In certain embodiments, sense oligomeric compounds consist of 18-30 linked nucleosides. In certain embodiments, sense oligomeric compounds consist of 19-30 linked nucleosides. In certain embodiments, sense oligomeric compounds consist of 16-25 linked nucleosides. In certain embodiments, sense oligomeric compounds consist of 18-25 linked nucleosides. In certain embodiments, sense oligomeric compounds consist of 18-20 linked nucleosides. In certain embodiments, sense oligomeric compounds consist of 19-21 linked nucleosides. In certain embodiments, sense oligomeric compounds consist of 18 linked nucleosides. In certain embodiments, sense oligomeric compounds consist of 19 linked nucleosides. In certain embodiments, sense oligomeric compounds consist of 20 linked nucleosides. In certain embodiments, sense oligomeric compounds consist of 21 linked nucleosides.

### D. Oligomeric Modifications

Provided oligomeric agents comprise one or more modifications, e.g., sugar, nucleobase, internucleoside linkage, and/or combinations thereof, incorporated into a modified oligonucleotide (e.g., an oligomeric compound). In certain embodiments, a modified oligonucleotide is characterized by modification motif(s) and overall length. In certain embodiments, such parameters are each independent of one another. Thus, unless otherwise indicated, each internucleoside linkage of an oligonucleotide having one or more modified sugar moiety and/or sugar motif, independently, is modified or unmodified and may or may not follow the modification pattern of the sugar modifications. For example, internucleoside linkages within regions of an oligonucleotide comprising certain sugar modifications may be the same or different from one another and may be the same or different from the internucleoside linkages of the region of the oligonucleotide comprising different sugar modifications. Likewise, such modified oligonucleotides may comprise one or more modified nucleobase independent of the pattern of the sugar modifications and independent of the internucleoside linkages. Unless specifically indicated, all modifications are independent of nucleobase sequence. Furthermore, each modification, whether internucleoside linkage, modified sugar moiety, modified nucleobase, of an antisense oligomeric compound is independent of each modification of a sense oligomeric compound binding partner unless specifically indicated otherwise.

### E. Nucleobase Sequence

In certain embodiments, modified oligonucleotides (e.g., oligomeric compounds) are further described by their nucleobase sequence. In certain embodiments oligonucleotides of oligomeric compounds have a nucleobase sequence that is complementary to a second oligonucleotide or an identified reference nucleic acid, such as a target nucleic acid. In certain such embodiments, a region of an oligonucleotide has a nucleobase sequence that is complementary to a second oligonucleotide or an identified reference nucleic acid, such as a target nucleic acid. In certain embodiments, the nucleobase sequence of a region or entire length of an oligonucleotide is at least 50%, at least 60%, at least 70%, at least 80%, at least 85%, at least 90%, at least 95%, or 100% complementary to the second oligonucleotide or nucleic acid, such as a target nucleic acid. In certain embodiments a first oligomeric compound comprises a modified oligonucleotide consisting of 18 to 50 linked nucleosides, wherein the nucleobase sequence of the first oligomeric compound comprises at least 18, at least 19, at least 20, at least 21, at least 22, or at least 23 contiguous nucleobases of the nucleobase sequence of any one of SEQ ID NOs: 2-5,

### II. Oligomeric Duplexes

In certain embodiments, an oligomeric compound provided herein comprises a modified oligonucleotide having a nucleobase sequence complementary to a sequence in a APOC3 target nucleic acid paired with a second oligomeric compound to form an oligomeric duplex. Such oligomeric duplex comprises a first oligomeric compound comprising a modified oligonucleotide having a portion complementary to a sequence in a APOC3 target nucleic acid and a second oligomeric compound comprising a modified oligonucleotide having a portion complementary to the first oligomeric compound. In certain embodiments, the first oligomeric compound of an oligomeric duplex comprises or consists of (1) a first modified oligonucleotide and optionally a conjugate group and/or terminal group; and the second oligomeric compound of the oligomeric duplex comprises or consists of (2) a second modified oligonucleotide and optionally a terminal group and/or a conjugate group. Either or both oligomeric compounds of an oligomeric duplex may comprise a conjugate group. Either or both oligomeric compounds of an oligomeric duplex may comprise a terminal group. The oligonucleotides of each oligomeric compound of an oligomeric duplex may include non-complementary or unpaired overhanging nucleosides. In certain embodiments the non-complementary or unpaired overhanging nucleosides are adenosine or thymine. In certain embodiments, the two oligonucleotides have at least one mismatch relative to one another. In certain embodiments, the oligomeric duplex is an antisense agent.

In certain embodiments, an oligomeric duplex comprises: a first oligomeric compound comprising a first modified oligonucleotide consisting of 18 to 28 linked nucleosides, wherein the nucleobase sequence of the first modified oligonucleotide comprises at least 18, at least 19, at least 20, at least 21, at least 22, or at least 23 contiguous nucleobases of the nucleobase sequence of any one of SEQ ID NOs: 2-5; and a second oligomeric compound comprising a second modified oligonucleotide consisting of 15 to 25 linked nucleosides, wherein the nucleobase sequence of the second modified oligonucleotide comprises at least 16, at least 17, at least 18, at least 19, at least 20, or at least 21 contiguous nucleobases of the nucleobase sequence of any one of SEQ ID NOs: 6-8; and wherein each of the nucleosides of the first modified oligonucleotide comprises a modified sugar moiety or sugar surrogate and wherein no more than 22%, no more than 20%, no more than 18%, no more than 15%, no more than 10%, or no more than 5% of the modified nucleosides in the first modified oligonucleotide comprises a 2′-F modification and each of the nucleosides of the second modified oligonucleotide comprises a modified sugar moiety or sugar surrogate and wherein no more than 25%, no more than 20%, no more than 18%, no more than 16%, no more than 14%, no more than 12%, or no more than 10%, of the modified nucleosides in the second modified oligonucleotide comprises a 2′-F modification. In certain embodiments, the oligomeric duplex is an antisense agent. In certain embodiments, the first oligomeric compound of the oligomeric duplex is an antisense agent, wherein the first modified oligonucleotide is an antisense oligomeric compound. In certain embodiments, the second oligomeric compound of the oligomeric duplex is a sense agent, wherein the second modified oligonucleotide is a sense oligomeric compound. In certain embodiments, the first modified oligonucleotide is an antisense RNAi oligonucleotide wherein no more than 22%, no more than 20%, no more than 18%, no more than 16%, no more than 14%, no more than 12%, no more than 10%, or no more than 7%, of the modified nucleosides in the oligomeric duplex comprise a modified sugar moiety comprising a 2′-F modification. In certain embodiments, the second modified oligonucleotide is a sense RNAi oligonucleotide. In certain embodiments, the nucleobase sequence of the second modified oligonucleotide is at least 90%, 95% or 100% complementary to the nucleobase sequence of an equal length portion of the first modified oligonucleotide.

In certain embodiments, an oligomeric duplex comprises a first oligomeric compound comprising a first modified oligonucleotide consisting of 18 to 28 linked nucleosides, wherein the nucleobase sequence of the first modified oligonucleotide comprises at least 18, at least 19, at least 20, at least 21, at least 22, or 23 contiguous nucleobases of the nucleobase sequence of any one of SEQ ID NO: 2-5; and a second oligomeric compound comprising a second modified oligonucleotide consisting of 15 to 25 linked nucleosides, wherein the nucleobase sequence of the second modified oligonucleotide comprises at least 16, at least 17, at least 18, at least 19, at least 20, or at least 21 contiguous nucleobases of the nucleobase sequence of any one of SEQ ID NOs: 6-8 and wherein each of the nucleosides of the first modified oligonucleotide comprises a modified sugar moiety or sugar surrogate and the first modified oligonucleotide comprises at least one modified nucleoside and no more than four modified nucleosides of the first modified oligonucleotide comprises a 2′-F modification, and each of the nucleosides of the second modified oligonucleotide comprises a modified sugar moiety or sugar surrogate and at least one modified nucleoside and no more than four modified nucleosides of the second modified oligonucleotide comprises a 2′-F modification. In certain embodiments, the first oligomeric compound is an antisense agent, wherein the first modified oligonucleotide is an antisense oligomeric compound. In certain embodiments, the second oligomeric compound is a sense agent, wherein the second modified oligonucleotide is a sense oligomeric compound. In certain embodiments, the first modified oligonucleotide is an antisense RNAi oligonucleotide. In certain embodiments, the second modified oligonucleotide is a sense RNAi oligonucleotide. In certain embodiments, the nucleobase sequence of the second modified oligonucleotide comprises a complementary region of at least 10, at least 11, at least 12, at least 13, at least 14, at least 15, at least 16, at least 17, at least 18, at least 19, at least 20, or 21 nucleobases that is 100% complementary to the nucleobase sequence of an equal portion of the first modified oligonucleotide. In certain embodiments, the nucleobase sequence of the second modified oligonucleotide is at least 80%, at least 85%, at least 90%, at least 95%, at least 98%, at least 99%, or 100% complementary to the nucleobase sequence of an equal portion of the first modified oligonucleotide. In certain embodiments, the oligomeric duplex is an antisense agent. In certain embodiments, the nucleobase sequence of the second modified oligonucleotide comprises a complementary region of at least 10, at least 11, at least 12, at least 13, at least 14, at least 15, at least 16, at least 17, at least 18, at least 19, at least 20, or 21 nucleobases that is 100% complementary to the nucleobase sequence of an equal portion of the first modified oligonucleotide; and the nucleobase sequence of the second modified oligonucleotide is at least 80%, at least 85%, at least 90%, at least 95%, at least 98%, at least 99%, or 100% complementary to the nucleobase sequence of an equal portion of the first modified oligonucleotide. In certain embodiments, the oligomeric duplex is an antisense agent, wherein no more than three nucleosides, no more than four nucleosides, no more than five nucleosides, no more than six nucleosides, no more than seven nucleosides, or no more than eight nucleosides in the oligomeric duplex comprise a modified sugar moiety or sugar surrogate comprising a fluorine.

In certain embodiments, an oligomeric duplex comprises a first oligomeric compound comprising a first modified oligonucleotide consisting of 18 to 28 linked nucleosides, wherein the nucleobase sequence of the first modified oligonucleotide comprises at least 10, at least 11, at least 12, at least 13, at least 14, at least 15, at least 16, at least 17, at least 18, at least 19, at least 20, or at least 21 contiguous nucleobases of the nucleobase sequence of any one of SEQ ID NO: 2-5; and a second oligomeric compound comprising a second modified oligonucleotide consisting of 15 to 25 linked nucleosides, wherein the nucleobase sequence of the second modified oligonucleotide comprises at least 10, at least 11, at least 12, at least 13, at least 14, at least 15, at least 16, at least 17, at least 18, at least 19, or at least 20 contiguous nucleobases the nucleobase sequence of any one of SEQ ID NO: 6-8; wherein each of the nucleosides of the first modified oligonucleotide independently and the second modified oligonucleotide independently comprises a modified sugar moiety or sugar surrogate independently selected from 2′-F, 2′-MOE, 2′-OMe, and 2′-deoxyribosyl, and wherein at least one modified nucleoside and no more than four modified nucleosides of the first modified oligonucleotide comprises a 2′-F modification, and at least one modified nucleoside and no more than four modified nucleosides of the second modified oligonucleotide comprises a 2′-F modification. In certain embodiments, the first oligomeric compound is an antisense agent, wherein the first modified oligonucleotide is an antisense oligomeric compound. In certain embodiments, the second oligomeric compound is a sense agent, wherein the second modified oligonucleotide is a sense oligomeric compound. In certain embodiments, the first modified oligonucleotide is an antisense RNAi oligonucleotide, and the second modified oligonucleotide is a sense RNAi oligonucleotide. In certain embodiments, the nucleobase sequence of the second modified oligonucleotide is at least 95% or 100% complementary to the nucleobase sequence of an equal length portion of the first modified oligonucleotide. In certain embodiments, the oligomeric duplex is an antisense agent wherein no more than three nucleosides, no more than four nucleosides, no more than five nucleosides, no more than six nucleosides, no more than seven nucleosides, or no more than eight nucleosides in the oligomeric duplex comprise a modified sugar moiety or sugar surrogate comprising a fluorine.

In certain embodiments, an oligomeric duplex comprises a first oligomeric compound comprising a first modified oligonucleotide consisting of 18 to 28 linked nucleosides, wherein the nucleobase sequence of the first modified oligonucleotide comprises at least 10, at least 11, at least 12, at least 13, at least 14, at least 15, at least 16, at least 17, at least 18, at least 19, at least 20, or at least 21 contiguous nucleobases of the nucleobase sequence of any one of SEQ ID NO: 2-5; and a second oligomeric compound comprising a second modified oligonucleotide consisting of 15 to 25 linked nucleosides, wherein the nucleobase sequence of the second modified oligonucleotide comprises at least 10, at least 11, at least 12, at least 13, at least 14, at least 15, at least 16, at least 17, at least 18, at least 19, or at least 20 contiguous nucleobases the nucleobase sequence of any one of SEQ ID NO: 6-8; wherein each of the nucleosides of the first modified oligonucleotide independently and the second modified oligonucleotide independently comprises a modified sugar moiety or sugar surrogate independently selected from 2′-F, 2′-MOE, 2′-OMe, and 2′-deoxyribosyl, wherein only one nucleoside or only three nucleosides of the first modified oligonucleotide are 2′-deoxynucleoside and no nucleosides of the second modified oligonucleotide are 2′-deoxynucleoside; wherein one or two of the modified sugar moiety and/or sugar surrogate comprises a 2′-F modification in the first modified oligonucleotide, and two modified nucleosides of the second modified oligonucleotide comprises a 2′-F modification. In certain embodiments, the first oligomeric compound is an antisense agent, wherein the first modified oligonucleotide is an antisense oligomeric compound comprising a 5′ terminal group. In certain embodiments, the second oligomeric compound is a sense agent, wherein the second modified oligonucleotide is a sense oligomeric compound optionally conjugated to a cell targeting moiety. In certain embodiments, the first modified oligonucleotide is an antisense RNAi oligonucleotide, and the second modified oligonucleotide is a sense RNAi oligonucleotide. In certain embodiments, the nucleobase sequence of the second modified oligonucleotide is at least 95% or 100% complementary to the nucleobase sequence of an equal length portion of the first modified oligonucleotide. In certain embodiments, the oligomeric duplex is an antisense agent wherein no more than three nucleosides, no more than four nucleosides, or no more than five nucleosides, in the oligomeric duplex comprise a modified sugar moiety or sugar surrogate comprising a fluorine.

In certain embodiments, an oligomeric duplex comprises a first oligomeric compound comprising a first modified oligonucleotide consisting of 18 to 28 linked nucleosides, wherein the nucleobase sequence of the first modified oligonucleotide comprises at least 10, at least 11, at least 12, at least 13, at least 14, at least 15, at least 16, at least 17, at least 18, at least 19, at least 20, or at least 21 contiguous nucleobases of the nucleobase sequence of any one of SEQ ID NO: 2-5; and a second oligomeric compound comprising a second modified oligonucleotide consisting of 15 to 25 linked nucleosides, wherein the nucleobase sequence of the second modified oligonucleotide comprises at least 10, at least 11, at least 12, at least 13, at least 14, at least 15, at least 16, at least 17, at least 18, at least 19, or at least 20 contiguous nucleobases the nucleobase sequence of any one of SEQ ID NO: 6-8; wherein each of the nucleosides of the first modified oligonucleotide independently and the second modified oligonucleotide independently comprises a modified sugar moiety or sugar surrogate independently selected from 2′-F, 2′-MOE, 2′-OMe, and 2′-deoxyribosyl, wherein only one nucleoside of the first modified oligonucleotide are 2′-deoxynucleoside and no nucleosides of the second modified oligonucleotide are 2′-deoxynucleoside; wherein three or four of the modified sugar moiety and/or sugar surrogate comprises a 2′-F modification in the first modified oligonucleotide, and two, three, or four modified nucleosides of the second modified oligonucleotide comprises a 2′-F modification. In certain embodiments, the first oligomeric compound is an antisense agent, wherein the first modified oligonucleotide is an antisense oligomeric compound comprising a 5′ terminal group. In certain embodiments, the second oligomeric compound is a sense agent, wherein the second modified oligonucleotide is a sense oligomeric compound optionally conjugated to a cell targeting moiety. In certain embodiments, the first modified oligonucleotide is an antisense RNAi oligonucleotide, and the second modified oligonucleotide is a sense RNAi oligonucleotide. In certain embodiments, the nucleobase sequence of the second modified oligonucleotide is at least 95% or 100% complementary to the nucleobase sequence of an equal length portion of the first modified oligonucleotide. In certain embodiments, the oligomeric duplex is an antisense agent wherein no more than five nucleosides, no more than six nucleosides, no more than seven nucleosides or no more than eight nucleosides, in the oligomeric duplex comprise a modified sugar moiety or sugar surrogate comprising a fluorine.

In certain embodiments, an oligomeric duplex comprises a first oligomeric compound comprising a first modified oligonucleotide, wherein the first modified oligonucleotide consists of 21-23 linked nucleosides and has nucleobase sequence comprising at least a 19-bp nucleobase sequence of any one of SEQ ID NOs: 2-5 having 0, 1, 2 or 3 nucleobases that are different from the corresponding nucleotide in any of SEQ ID NOs: 2-5; and a second oligomeric compound comprising a second modified oligonucleotide wherein the second modified oligonucleotide consists of 19-21 linked nucleosides and has nucleobase sequence comprising at least a 17-bp nucleobase sequence of any one of SEQ ID NOs: 6-8 having 0, 1, 2 or 3 nucleobases that are different from the corresponding nucleotide in any of SEQ ID NOs: 6-8, wherein each of the nucleosides of the first modified oligonucleotide independently and the second modified oligonucleotide independently comprises a modified sugar moiety or sugar surrogate independently selected from 2′-F, 2′-MOE, 2′-OMe, and 2′-deoxyribosyl, wherein three nucleosides or no nucleosides of the first modified oligonucleotide is 2′-deoxynucleoside and no nucleosides of the second modified oligonucleotide are 2′-deoxynucleoside; wherein one or two or three or four of the modified sugar moiety and/or sugar surrogate comprises a 2′-F modification in the first modified oligonucleotide, and two, or four modified nucleosides of the second modified oligonucleotide comprises a 2′-F modification. In certain embodiments, the first oligomeric compound is an antisense agent, wherein the first modified oligonucleotide is an antisense oligomeric compound comprising a 5′ terminal group. In certain embodiments, the second oligomeric compound is a sense agent, wherein the second modified oligonucleotide is a sense oligomeric compound optionally conjugated to a cell targeting moiety. In certain embodiments, the first modified oligonucleotide is an antisense RNAi oligonucleotide, and the second modified oligonucleotide is a sense RNAi oligonucleotide. In certain embodiments, the nucleobase sequence of the second modified oligonucleotide is at least 95% or 100% complementary to the nucleobase sequence of an equal length portion of the first modified oligonucleotide. In certain embodiments, the oligomeric duplex is an antisense agent, wherein at least one modified nucleoside and no more than five nucleosides, no more than six nucleosides, no more than seven nucleosides or no more than eight nucleosides, in the oligomeric duplex comprise a modified sugar moiety or sugar surrogate comprising a fluorine. In certain embodiments, one or two of the modified sugar moiety and/or sugar surrogate comprises a 2′-F modification in the first modified oligonucleotide, and two modified nucleosides of the second modified oligonucleotide comprises a 2′-F modification, and no more than three nucleosides in the oligomeric duplex comprise a modified sugar moiety or sugar surrogate comprising a fluorine. In certain embodiments, three or four of the modified sugar moiety and/or sugar surrogate comprises a 2′-F modification in the first modified oligonucleotide, and two or four modified nucleosides of the second modified oligonucleotide comprises a 2′-F modification. In certain embodiments, the oligomeric duplex comprises one or two unpaired nucleosides at either or both ends, forming one or two overhang ends. In certain embodiments an overhang end is one or two nucleosides of the antisense oligomeric compound. In certain embodiments an overhang end is one or two 3′-nucleosides of the antisense oligomeric compound. In certain embodiments the last two 3′-nucleosides of the antisense oligomeric compound are overhang nucleosides not paired with the sense oligomeric compound. In certain embodiments the last one or two 3′-unpaired overhang nucleosides comprise an adenine nucleobase. In certain embodiments the last one or two 3′-unpaired overhang nucleosides comprise a thymine nucleobase. In certain embodiments the last one or two 3′-unpaired overhang nucleosides comprise a uridine nucleobase. In certain embodiments the last one or two 3′-unpaired overhang nucleosides comprise an inosine nucleobase. In certain embodiments, the oligomeric duplex is an antisense agent wherein no more than five nucleosides, no more than six nucleosides, no more than seven nucleosides or no more than eight nucleosides, in the oligomeric duplex comprise a modified sugar moiety or sugar surrogate comprising a fluorine.

In certain embodiments, an oligomeric duplex comprises a first oligomeric compound comprising a first modified oligonucleotide, wherein the first modified oligonucleotide consists of 21-23 linked nucleosides and has a nucleobase sequence comprising at least a 19-bp sequence of any one of SEQ ID NOs: 11-34 and 51-74, having 0, 1, 2 or 3 mismatches with a sequence in a target APOC3 nucleic acid sequence; and a second oligomeric compound comprising a second modified oligonucleotide wherein the second modified oligonucleotide consists of 19-21 linked nucleosides, comprising at least a 19-bp sequence of any one of SEQ ID NOs: 35-49 and 75-89, having 0, 1, 2 or 3 mismatches to the first modified oligonucleotide. In certain embodiments, the first modified oligonucleotide is an antisense RNAi oligonucleotide, and the second modified oligonucleotide is a sense RNAi oligonucleotide. In certain embodiments, the nucleobase sequence of the second modified oligonucleotide is at least 95% or 100% complementary to the nucleobase sequence of an equal length portion of the first modified oligonucleotide. In certain embodiments, the oligomeric duplex is an antisense agent wherein each of the nucleosides of the first modified oligonucleotide independently and the second modified oligonucleotide independently comprises a modified sugar moiety or sugar surrogate independently selected from 2′-F, 2′-MOE, 2′-OMe, and 2′-deoxyribosyl, and wherein at least one modified nucleoside and no more than four modified nucleosides of the first modified oligonucleotide comprises a 2′-F modification, and at least one modified nucleoside and no more than four modified nucleosides of the second modified oligonucleotide comprises a 2′-F modification. In certain embodiments, the oligomeric duplex comprises one or two unpaired nucleosides at either or both ends, forming one or two overhang ends. In certain embodiments an overhang end is one or two nucleosides of the antisense oligomeric compound. In certain embodiments an overhang end is one or two 3′-nucleosides of the antisense oligomeric compound. In certain embodiments the last two 3′-nucleosides of the antisense oligomeric compound are overhang nucleosides not paired with the sense oligomeric compound. In certain embodiments the last one or two 3′-unpaired overhang nucleosides comprise an adenine nucleobase. In certain embodiments the last one or two 3′-unpaired overhang nucleosides comprise a thymine nucleobase. In certain embodiments the last one or two 3′-unpaired overhang nucleosides comprise a uridine nucleobase. In certain embodiments the last one or two 3′-unpaired overhang nucleosides comprise a inosine nucleobase. In certain embodiments the last two 3′-unpaired overhang nucleosides comprise a thymine nucleobase and an inosine nucleobase. In certain embodiments the last two 3′-unpaired overhang nucleosides comprise 5′ to 3′ a thymine nucleobase and an inosine nucleobase. In certain embodiments the last two 3′-unpaired overhang nucleosides comprise 5′ to 3′ an inosine nucleobase and a thymine nucleobase. In certain embodiments the last two 3′-unpaired overhang nucleosides comprise 5′ to 3′ an adenine nucleobase and an inosine nucleobase. In certain embodiments the last two 3′-unpaired overhang nucleosides comprise 5′ to 3′ an inosine nucleobase and an adenine nucleobase.

In certain embodiments, an oligomeric duplex comprises a first oligomeric compound comprising a first modified oligonucleotide, wherein the first modified oligonucleotide consists of 21-23 linked nucleosides and has a nucleobase sequence comprising at least a 19-bp sequence of any one of SEQ ID NOs: 11-34, having 0, 1, 2 or 3 mismatches with a sequence in a target APOC3 nucleic acid sequence; and a second oligomeric compound comprising a second modified oligonucleotide wherein the second modified oligonucleotide consists of 19-21 linked nucleosides, comprising at least a 19-bp sequence of any one of SEQ ID NOs: 35-49, having 0, 1, 2 or 3 mismatches to the first modified oligonucleotide. In certain embodiments, the first modified oligonucleotide is an antisense RNAi oligonucleotide, and the second modified oligonucleotide is a sense RNAi oligonucleotide. In certain embodiments, the nucleobase sequence of the second modified oligonucleotide is at least 95% or 100% complementary to the nucleobase sequence of an equal length portion of the first modified oligonucleotide. In certain embodiments, the oligomeric duplex is an antisense agent wherein each of the nucleosides of the first modified oligonucleotide independently and the second modified oligonucleotide independently comprises a modified sugar moiety or sugar surrogate independently selected from 2′-F, 2′-MOE, 2′-OMe, and 2′-deoxyribosyl, and wherein at least one modified nucleoside and no more than four modified nucleosides of the first modified oligonucleotide comprises a 2′-F modification, and at least one modified nucleoside and no more than four modified nucleosides of the second modified oligonucleotide comprises a 2′-F modification. In certain embodiments, the oligomeric duplex comprises one or two unpaired nucleosides at either or both ends, forming one or two overhang ends. In certain embodiments an overhang end is one or two nucleosides of the antisense oligomeric compound. In certain embodiments an overhang end is one or two 3′-nucleosides of the antisense oligomeric compound. In certain embodiments the last two 3′-nucleosides of the antisense oligomeric compound are overhang nucleosides not paired with the sense oligomeric compound. In certain embodiments the last one or two 3′-unpaired overhang nucleosides comprise an adenine nucleobase. In certain embodiments the last one or two 3′-unpaired overhang nucleosides comprise a thymine nucleobase. In certain embodiments the last one or two 3′-unpaired overhang nucleosides comprise a uridine nucleobase. In certain embodiments the last one or two 3′-unpaired overhang nucleosides comprise an inosine nucleobase. In certain embodiments the antisense oligomeric compound comprises a 5′-terminal group. In certain embodiments the sense strand comprises a conjugate group attached at the 5′ or 3′ end of the sense oligomeric compound.

In certain embodiments, an oligomeric duplex comprises a first oligomeric compound comprising a first modified oligonucleotide consisting of 19 to 25 linked nucleosides and a second oligomeric compound comprising a second modified oligonucleotide consisting of 16 to 24 linked nucleosides, wherein the nucleobase sequence of the first modified oligonucleotide and the nucleobase sequence of the second modified oligonucleotide each comprises at least 8, at least 9, at least 10, at least 11, at least 12, at least 13, at least 14, at least 15, at least 16, at least 17, at least 18, at least 19, at least 20, at least 21, at least 22, or at least 23 contiguous nucleobases of any of the following pairs selected from a first oligomeric compound selected from any one of SEQ ID NOs: 11-34 and 51-74 and a second oligomeric compound selected from any one of SEQ ID NOS: 35-49 and 75-89. In certain embodiments, the first oligomeric compound is an antisense agent. In certain embodiments, the first modified oligonucleotide is an antisense oligomeric compound. In certain embodiments, the second oligomeric compound is a sense agent. In certain embodiments, the second modified oligonucleotide is a sense oligomeric compound. In certain embodiments, the first modified oligonucleotide is an antisense RNAi oligonucleotide. In certain embodiments, the second oligomeric compound is a sense agent. In certain embodiments, the second modified oligonucleotide is a sense RNAi oligonucleotide.

In certain embodiments, an oligomeric duplex comprises a first oligomeric compound comprising a first modified oligonucleotide consisting of 21 or 23 linked nucleosides and a second oligomeric compound comprising a second modified oligonucleotide consisting of 19 or 21 linked nucleosides, wherein the nucleobase sequences of the first modified oligonucleotide and second modified oligonucleotide consist of any of the following pairs of selected from a first oligomeric compound selected from any one of SEQ ID NOs: 11-34 and a second oligomeric compound selected from any one of SEQ ID NOS: 35-49. In certain embodiments, the first oligomeric compound is an antisense agent. In certain embodiments, the first modified oligonucleotide is an antisense oligomeric compound. In certain embodiments, the second oligomeric compound is a sense agent. In certain embodiments, the second modified oligonucleotide is a sense oligomeric compound. In certain embodiments, the first modified oligonucleotide is an antisense RNAi oligonucleotide. In certain embodiments, the second oligomeric compound is a sense agent. In certain embodiments, the second modified oligonucleotide is a sense RNAi oligonucleotide. In certain embodiments, the oligomeric duplex comprises one or two unpaired nucleosides at either or both ends, forming one or two overhang ends. In certain embodiments an overhang end is one or two 3′-nucleosides of the antisense oligomeric compound. In certain embodiments the last two 3′-nucleosides of the antisense oligomeric compound are overhang nucleosides not paired with the sense oligomeric compound. In certain embodiments the last one or two 3′-unpaired overhang nucleosides comprise an adenine nucleobase. In certain embodiments the last one or two 3′-unpaired overhang nucleosides comprise a thymine nucleobase. In certain embodiments the last one or two 3′-unpaired overhang nucleosides comprise a uridine nucleobase. In certain embodiments the last one or two 3′-unpaired overhang nucleosides comprise an inosine nucleobase. In certain embodiments the antisense oligomeric compound comprises a 5′-terminal group. In certain embodiments the sense strand comprises a conjugate group attached at the 5′ or 3′ end of the sense oligomeric compound.

In any of the oligomeric duplexes described herein, at least one nucleoside of the first modified oligonucleotide and/or the second modified oligonucleotide comprises a modified sugar moiety. Examples of suitable modified sugar moieties include, but are not limited to, a bicyclic sugar moiety, such as a 2′-4′ bridge selected from —O—CH₂—; and —O—CH(CH₃)—, and a non-bicyclic sugar moiety, such as a 2′-MOE sugar moiety, a 2′-F sugar moiety, a 2′-OMe sugar moiety, or a 2′-NMA sugar moiety. In certain embodiments, at least one nucleoside of the first modified oligonucleotide and/or the second modified oligonucleotide comprises a modified 2′-deoxyribosyl sugar moiety. In certain embodiments, at least 80%, at least 90%, or 100% of the nucleosides of the first modified oligonucleotide and/or the second modified oligonucleotide comprises a modified sugar moiety independently selected from 2′-F, 2′-MOE, 2′-OMe, and 2′-deoxyribosyl. In certain embodiments, at least 80%, at least 90%, or 100% of the nucleosides of the first modified oligonucleotide and the second modified oligonucleotide comprises a modified sugar moiety independently selected from 2′-F, 2′-MOE, 2′-OMe, and 2′-deoxyribosyl.

In certain embodiments, in an oligomeric duplex provided herein, at least one nucleoside of the first modified oligonucleotide and/or the second modified oligonucleotide comprises a modified sugar moiety and/or sugar surrogate. In certain embodiments, in an oligomeric duplex provided herein, a sugar moiety of the first modified oligonucleotide is modified, wherein the modified sugar moiety and/or sugar surrogate is selected from 2′-F, 2′-MOE, 2′-OMe, and 2′-deoxyribosyl. In certain embodiments, in an oligomeric duplex provided herein, a sugar motif (from 5′ to 3′) of the first modified oligonucleotide is selected from: efyyyfyyyyyyyfyfyyyyyyy, efyyydyyyyyyyfyfyyyyyyy, efyyydyyyyyyydydyyyyyyy, efyyyyyyyyyyyfyyyyyyyyy, efyyyfyyeyyyyfyfyyyyyyy, efyyyfyyeeyyyfYyyyyyy, efyyydyyyyyyyfyfyyyyyee, efyyydyyyyyyydydyyyyyee, efyyydyyeyyyyfyfyyyyyee, efyyydyyeyyyydydyyyyyee, efyyydyyeeyyydydyyyyyee, efyyydyyeeyyyfyfyyyyyee, efyyyfyyyyyyyfyfyyyee, efyyydyyyyyyyfyfyyyee, and efyyydyyyyyyydydyyyee, wherein each ‘y’ represents a 2′-OMe sugar moiety, each ‘d’ represents a 2′-β-D-deoxyribosyl sugar moiety, and each ‘e’ represents a 2′-MOE sugar moiety. In certain embodiments, in an oligomeric duplex provided herein, a sugar moiety of the second modified oligonucleotide is modified, wherein the modified sugar moiety is selected from 2′-F, 2′-MOE, 2′-OMe, and 2′-deoxyribosyl. In certain embodiments, in an oligomeric duplex provided herein, a sugar motif (from 5′ to 3′) of the second modified oligonucleotide is selected from among: yyyyyyfyfffyyyyyyyyyy, yyyyyyyyyffyyyyyyyyyy, yyyyyyyfy fyyyyyyyyy, eeyyyyyyyffyyyyyyyyee, eeyyyyyyffyyyyyyyyyee, yyyyyyfyfffyyyyyyyy, yyyyyyfyfyyyyyyyyyy, yyyyyyyfyfyyyyyyyy, yyyyyyyyfyf yyyyyyy, eeyyyyyyyffyyyyyyee, eeyyyyyyffyyyyyyyee, and dyyyyyyfyfffyyyyyyyyyy, wherein each ‘y’ represents a 2′-OMe sugar moiety, each ‘f’ represents a 2′-F sugar moiety, each ‘d’ represents a 2′-β-D-deoxyribosyl sugar moiety, and each ‘e’ represents a 2′-MOE sugar moiety.

In certain embodiments, in an oligomeric duplex provided herein, at least one internucleoside linkage of the first modified oligonucleotide and/or the second modified oligonucleotide comprises a modified internucleoside linkage. In certain embodiments, the modified internucleoside linkage is a phosphorothioate internucleoside linkage. In certain embodiments, at least one of the first, second, or third internucleoside linkages from the 5′ end and/or the 3′ end of the first modified oligonucleotide comprises a phosphorothioate linkage. In certain embodiments, at least one of the first, second, or third internucleoside linkages from the 5′ end and/or the 3′ end of the second modified oligonucleotide comprises a phosphorothioate linkage.

In certain embodiments, in an oligomeric duplex provided herein, each internucleoside linkage of the first modified oligonucleotide is independently selected from a phosphodiester and a phosphorothioate, internucleoside linkage, and each internucleoside linkage of the second modified oligonucleotide is independently selected from a phosphodiester and a phosphorothioate, internucleoside linkage.

In certain embodiments, in an oligomeric duplex provided herein, at least one linkage of the antisense oligomeric compound is a modified linkage. In certain embodiments, in an oligomeric duplex provided herein, an internucleoside linkage of the first modified oligonucleotide is modified, wherein the 5′-most internucleoside linkage (i.e., linking the first nucleoside from the 5′-end to the second nucleoside from the 5′-end) is modified. In certain embodiments, in an oligomeric duplex provided herein, the internucleoside linkage motif (from 5′ to 3′) of the first modified oligonucleotide is selected from 5′-ssooooooooooooooooooss-3′, 5′-ssooosooooooooooooooss-3′, 5′-ssooosooooooososooooss-3′, 5′-ssooooooooooooooooss-3′, 5′-ssooosooooooooooooss-3′, 5′-ssooosooooooososooss-3′, wherein each “s” is a phosphorothioate internucleoside internucleoside linkage and each “o” is a phosphodiester internucleoside linkage. In certain embodiments, in an oligomeric duplex provided herein, an internucleoside linkage of the second modified oligonucleotide is modified, wherein the 5′-most internucleoside linkage (i.e., linking the first nucleoside from the 5′-end to the second nucleoside from the 5′-end) is modified. In certain embodiments, in an oligomeric duplex provided herein, the internucleoside linkage motif (from 5′ to 3′) of the second modified oligonucleotide is selected from (from 5′ to 3′) of: 5′ssooooooosooooooooss-3′, 5′-ssoooooosoooooooooss-3′, 5′-ssooooooooooooooss-3′, 5′-ssooooooosooooooss-3′, 5′-ssoooooosoooooooss-3′, 5′-ossooooooooooooooooss-3′ wherein each ‘o’ represents a phosphodiester internucleoside linkage and each ‘s’ represents a phosphorothioate internucleoside linkage. In certain embodiments, the two 5′-most internucleoside linkages are modified. In certain embodiments, the first one or 2 internucleoside linkages from the 3′-end are modified. In certain embodiments, the modified internucleoside linkage is a phosphorothioate linkage.

In certain embodiments, in an oligomeric duplex provided herein, at least one nucleobase of the first modified oligonucleotide and/or at least one nucleobase of the second modified oligonucleotide is a modified nucleobase. In certain embodiments, the modified nucleobase is inosine.

In certain embodiments, in an oligomeric duplex provided herein, the first oligomeric compound comprises a terminal group comprising a stabilized phosphate group attached to the 5′ position of the 5′-most nucleoside. In certain embodiments, the stabilized phosphate group comprises a cyclopropyl phosphonate or an (E)-vinyl phosphonate. In certain embodiments, the stabilized phosphate group is an (E)-vinyl phosphonate.

In certain embodiments, in an oligomeric duplex provided herein, the first modified oligonucleotide is attached to a conjugate group. In certain embodiments, the conjugate group comprises a conjugate linker and a conjugate moiety. In certain embodiments, the conjugate group is attached to the first modified oligonucleotide at the 5′-end of the first modified oligonucleotide. In certain embodiments, the conjugate group is attached to the first modified oligonucleotide at the 3′-end of the modified oligonucleotide. In certain embodiments, the conjugate group is attached to the first modified oligonucleotide at an internal position. In certain embodiments, the conjugate group is attached to the first modified oligonucleotide through a 2′-modification of a furanosyl sugar moiety. In certain embodiments, the conjugate group is attached to the first modified oligonucleotide through a modified internucleoside linkage. In certain embodiments, the conjugate group comprises N-acetyl galactosamine. In certain embodiments, a conjugate group comprises a moiety selected from any of a C22 alkyl, C20 alkyl, C16 alkyl, C10 alkyl, C21 alkyl, C19 alkyl, C18 alkyl, C17 alkyl, C15 alkyl, C14 alkyl, C13 alkyl, C12 alkyl, C11 alkyl, C9 alkyl, C8 alkyl, C7 alkyl, C6 alkyl, C5 alkyl, C22 alkenyl, C20 alkenyl, C16 alkenyl, C10 alkenyl, C21 alkenyl, C19 alkenyl, C18 alkenyl, C17 alkenyl, C15 alkenyl, C14 alkenyl, C13 alkenyl, C12 alkenyl, C11 alkenyl, C9 alkenyl, C8 alkenyl, C7 alkenyl, C6 alkenyl, or C5 alkenyl. In certain embodiments, a conjugate group comprises a moiety selected from any of C22 alkyl, C20 alkyl, C16 alkyl, C10 alkyl, C21 alkyl, C19 alkyl, C18 alkyl, C17 alkyl, C15 alkyl, C14 alkyl, C13 alkyl, C12 alkyl, C11 alkyl, C9 alkyl, C8 alkyl, C7 alkyl, C6 alkyl, and C5 alkyl, where the alkyl chain optionally has one or more unsaturated bonds.

In any of the oligomeric duplexes described herein, the second modified oligonucleotide optionally is attached to a conjugate group. In certain embodiments, the conjugate group comprises a conjugate linker and a conjugate moiety. In certain embodiments, the conjugate group is attached to the second modified oligonucleotide at the 5′-end of the second modified oligonucleotide. In certain embodiments, the conjugate group is attached to the second modified oligonucleotide at the 3′-end of the modified oligonucleotide. In certain embodiments, the conjugate group is attached to the second modified oligonucleotide at an internal position. In certain embodiments, the conjugate group is attached to the second modified oligonucleotide through a 2′-modification of a furanosyl sugar moiety. In certain embodiments, the conjugate group is attached to the second modified oligonucleotide through a modified internucleoside linkage. In certain embodiments, the conjugate group comprises N-acetyl galactosamine. In certain embodiments, an oligomeric agent comprises an antisense agent, which comprises an oligomeric duplex described herein. In certain embodiments, an antisense agent, which is an oligomeric duplex described herein, is an RNAi agent capable of reducing the amount of APOC3 RNA through the activation of RISC/Ago2.

In certain embodiments, an oligomeric agent comprises at least two oligomeric duplexes linked together. In certain embodiments, an oligomeric agent comprises two oligomeric duplexes wherein at least one oligomeric duplex is targeted to APOC3 RNA as described herein. In certain embodiments, an oligomeric agent comprises two or more of the same oligomeric duplex, which is any of the oligomeric duplexes described herein. In certain embodiments, the two or more oligomeric duplexes are covalently linked together. In certain embodiments, the second modified oligonucleotides of the two or more oligomeric duplexes are covalently linked together. In certain embodiments, the second modified oligonucleotides of two or more oligomeric duplexes are covalently linked together at their 3′ ends. In certain embodiments, the second modified oligonucleotides of two or more oligomeric duplexes are covalently linked together at the 3′ end of one to the 5′ end of the other. In certain embodiments, the two or more oligomeric duplexes are covalently linked together by a glycol linker, such as a tetraethylene glycol linker. A structure of oligomeric duplexes covalently linked by a glycol linker is described in, e.g., Alterman, et al., Nature Biotech., 37:844-894, 2019. In certain embodiments, a first modified oligonucleotide of a first oligomeric duplex is covalently linked to a second modified oligonucleotide of a second oligomeric duplex and a first modified oligonucleotide of the second oligomeric duplex is covalently linked to a second modified oligonucleotide of the first oligomeric duplex (see, e.g., PCT International Patent Application Publication WO2020/065602 for a description of an example of a structure of linked oligomeric duplexes).

### III. Conjugates

In certain embodiments, provided herein are oligomeric compounds comprising one or more modified oligonucleotide and one or more conjugate groups. In certain embodiments, an oligomeric compound optionally further comprises one or more terminal groups. Conjugate groups comprise or consist of a conjugate moiety and a conjugate linker. A conjugate group may be attached at the 3′ end and/or the 5′ end of an oligonucleotide and/or at any internal position. In certain embodiments, conjugate groups are attached through a modified sugar moiety or a modified internucleoside linkage. In certain embodiments, oligomeric compounds comprise a modified oligonucleotide, a cell-targeting moiety, and a conjugate linker.

### A. Conjugate Groups

In certain embodiments, a conjugate group comprises a conjugate moiety and a conjugate linker.

### Conjugate Moieties

In certain embodiments, a conjugate moiety modifies one or more properties of an attached oligonucleotide, including but not limited to pharmacodynamics, pharmacokinetics, stability, binding, absorption, tissue distribution, cellular distribution, cellular uptake, charge and clearance. In certain embodiments, a conjugate moiety imparts a new property on the attached oligonucleotide.

In certain embodiments, a conjugate moiety comprises or consists of a cell-targeting moiety. In certain embodiments, a cell-targeting moiety is capable of binding the cell-surface receptor or the cell-surface moiety. In certain embodiments, an agent comprising a cell-targeting moiety is capable of being internalized when it interacts with or binds the cell-surface receptor or the cell-surface moiety. In certain embodiments, a cell-targeting moiety comprises a liver cell targeting moiety or a liver cell ligand. In certain embodiments, a liver cell-targeting moiety consists of a cell-targeting moiety having affinity for the hepatic asialoglycoprotein receptor (ASGP-R). In certain embodiments, the cell-targeting moiety comprises more than one ligand, and each ligand has affinity for the ASGP-R. In certain embodiments, each ligand is a carbohydrate. In certain embodiments, each ligand is independently selected from galactose, N-acetyl galactosamine (GalNAc), mannose, glucose, glucosamine, and fucose.

In certain embodiments, each ligand of a cell-targeting moiety is a carbohydrate, carbohydrate derivative, modified carbohydrate, polysaccharide, modified polysaccharide, or polysaccharide derivative. In certain such embodiments, the conjugate group comprises a carbohydrate cluster (see, e.g., Maier et al., “Synthesis of Antisense Oligonucleotides Conjugated to a Multivalent Carbohydrate Cluster for Cellular Targeting,” Bioconjugate Chemistry, 2003, 14, 18-29 or Rensen et al., “Design and Synthesis of Novel N-Acetylgalactosamine-Terminated Glycolipids for Targeting of Lipoproteins to the Hepatic Asiaglycopro-tein Receptor,” J. Med. Chem. 2004, 47, 5798-5808). In certain such embodiments, each ligand is an amino sugar or a thio sugar. For example, amino sugars may be selected from any number of compounds known in the art, such as sialic acid, α-D-galactosamine, β-muramic acid, 2-deoxy-2-methylamino-L-glucopyranose, 4,6-dideoxy-4-formamido-2,3-di-O-methyl-D-mannopyranose, 2-deoxy-2-sulfoamino-D-glucopyranose and N-sulfo-D-glucosamine, and N-glycoloyl-α-neuraminic acid. For example, thio sugars may be selected from 5-Thio-β-D-glucopyranose, methyl 2,3,4-tri-O-acetyl-1-thio-6-O-trityl-α-D-glucopyranoside, 4-thio-β-D-galactopyranose, and ethyl 3,4,6,7-tetra-O-acetyl-2-deoxy-1,5-dithio-α-D-gluco-heptopyranoside.

In certain embodiments, each ligand is N-acetyl galactosamine (GalNAc). In certain embodiments, the cell-targeting moiety comprises one GalNAc ligand. In certain embodiments, the cell-targeting moiety comprises two GalNAc ligands. In certain embodiments, the cell-targeting moiety comprises three GalNAc ligands. In certain embodiments, the cell-targeting moiety comprises a GalNAc ligand cluster. In certain embodiments, the cell-targeting moiety comprises a three GalNAc ligand cluster. In certain embodiments, the cell-targeting moiety is any one of those described in U.S. Pat. No. 9,127,276, the entire contents of which is incorporated herein by reference. In certain embodiments, a conjugate groups comprises a cell-targeting moiety selected from any one of the formula set forth in TABLE A:

TABLE A
Conjugate Groups

| | |
|---|---|
| 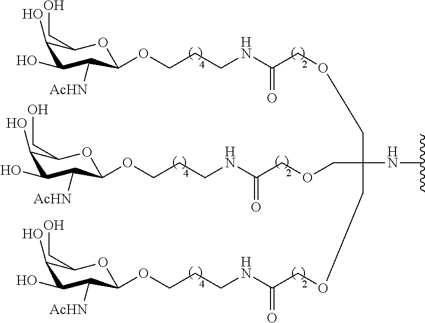 | GalNAc3-7 |
| 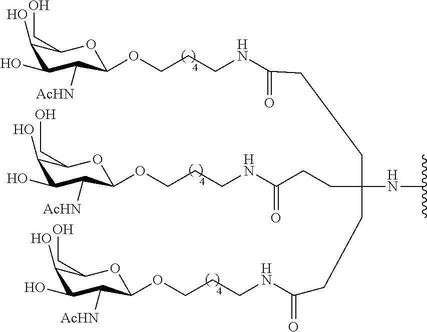 | GalNAc3-10 |
| 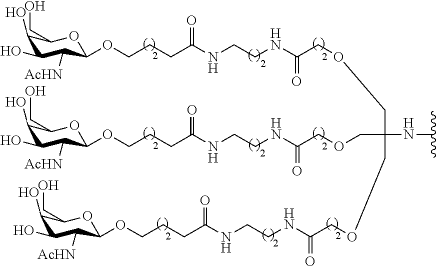 | GalNAc3-1 |
| 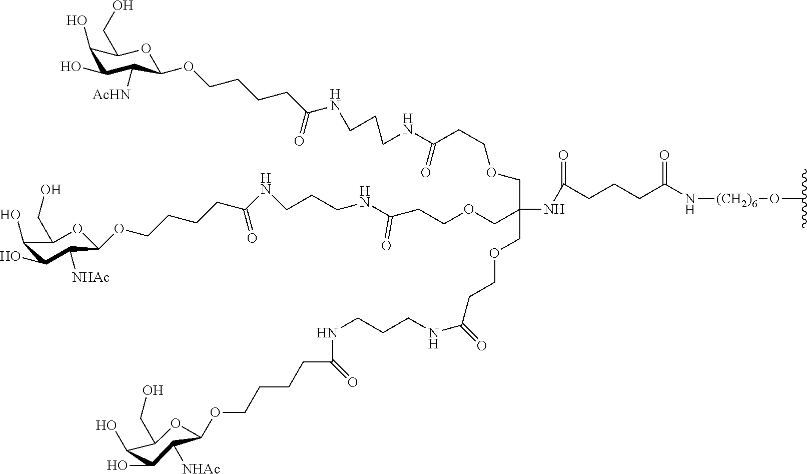 | GalNAc3-3 |
| 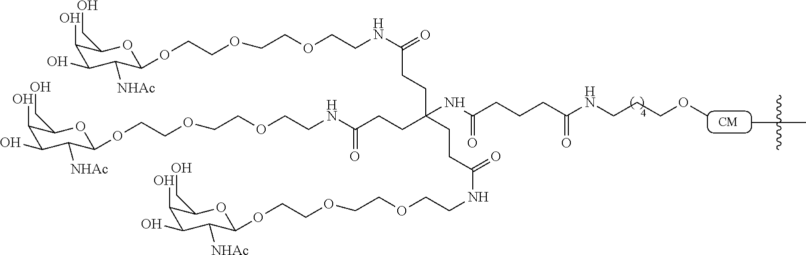 | GalNAc3-23 (with a cleavable linker moiety) |
| 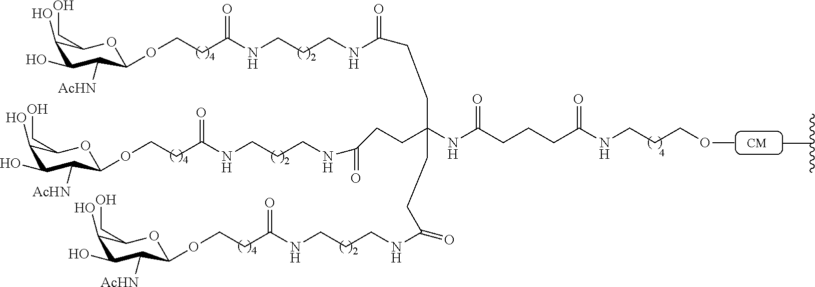 | GalNAc3-8 (with a cleavable linker moiety) |
| 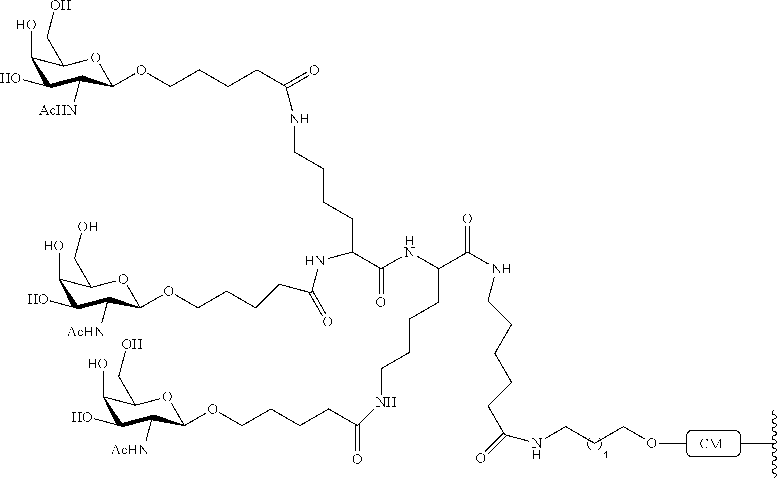 | GalNAc3-5 (with a cleavable linker moiety) |
| 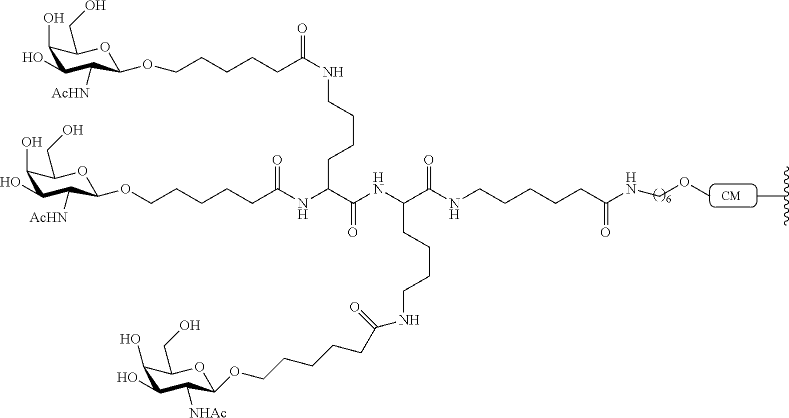 | GalNAc3-13 (with a cleavable linker) |
| 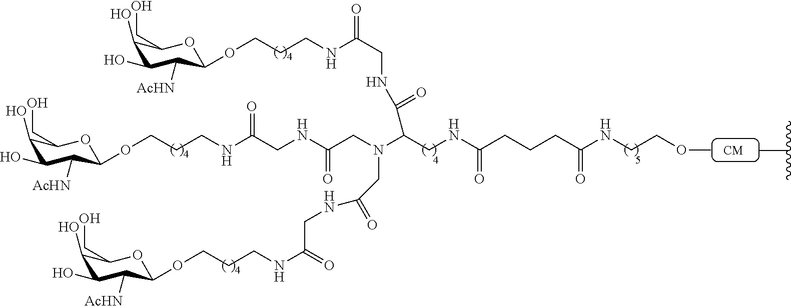 | GalNAc3-6 (with a cleavable linker moiety) |
| 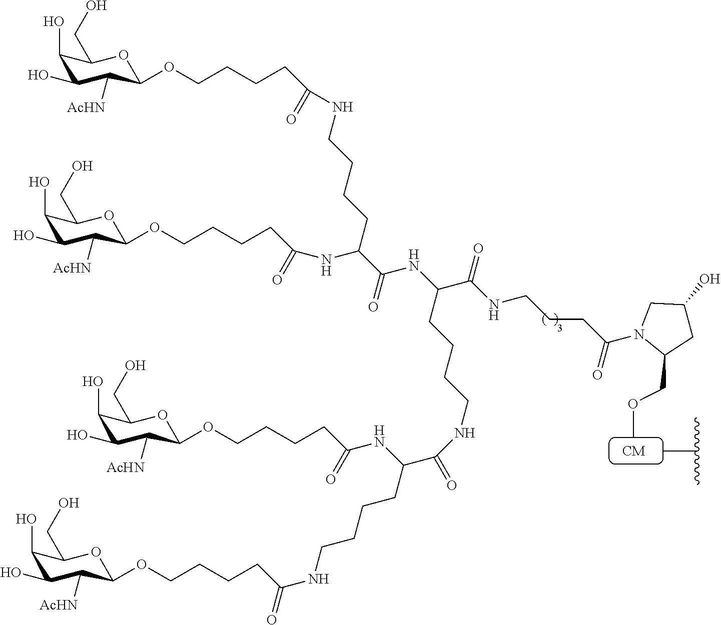 | GalNAc4-11 (with a cleavable linker moiety) |
| 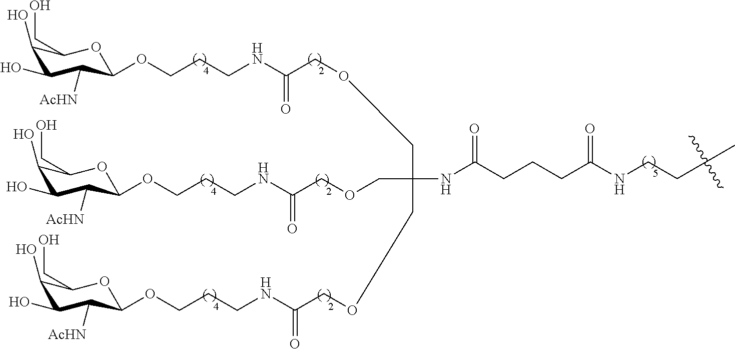 | LICA-1 |

### Conjugate Linkers

In certain embodiments, oligomeric compounds comprise an oligonucleotide and a conjugate group, wherein the conjugate group comprises a conjugate moiety and a conjugate linker. In certain embodiments, the conjugate linker links the conjugate moiety to the oligonucleotide. In certain embodiments, the conjugate linker is a single chemical bond (i.e., the conjugate moiety is attached directly to an oligonucleotide through a single bond). In certain embodiments, the conjugate linker comprises one or more atoms. In certain embodiments, the conjugate linker comprises a chemical group. In certain embodiments, the conjugate linker comprises a chain structure, such as a hydrocarbyl chain, or an oligomer of repeating units such as ethylene glycol, nucleosides, or amino acid units. In certain embodiments, the oligonucleotide is a modified oligonucleotide. In certain embodiments, the conjugate moiety is a bicycle ligand. In certain embodiments, the conjugate moiety comprises two peptide loops attached to a molecular scaffold.

In certain embodiments, a conjugate linker comprises one or more groups selected from alkyl, amino, oxo, amide, disulfide, polyethylene glycol, ether, thioether, and hydroxylamino. In certain such embodiments, the conjugate linker comprises one or more groups selected from alkyl, amino, oxo, amide and ether groups. In certain embodiments, the conjugate linker comprises one or more groups selected from alkyl and amide groups. In certain embodiments, the conjugate linker comprises one or more groups selected from alkyl and ether groups. In certain embodiments, the conjugate linker comprises at least one phosphorus moiety. In certain embodiments, the conjugate linker comprises at least one phosphate group. In certain embodiments, the conjugate linker includes at least one neutral linking group.

In certain embodiments, conjugate linkers, including the conjugate linkers described herein, are bifunctional linking moieties, e.g., those known in the art to be useful for attaching conjugate moieties to parent compounds, such as the oligonucleotides provided herein. In general, a bifunctional linking moiety comprises at least two functional groups. One of the functional groups is selected to react with a particular site on a parent compound and the other is selected to react with a peptide extender. Examples of functional groups used in a bifunctional linking moiety include but are not limited to electrophiles for reacting with nucleophilic groups and nucleophiles for reacting with electrophilic groups. In certain embodiments, bifunctional linking moieties comprise one or more groups selected from amino, hydroxyl, carboxylic acid, thiol, alkyl, alkenyl, and alkynyl.

In certain embodiments, conjugate linkers comprise chemical groups that are formed upon a reaction between a first functional group and a second functional group. In certain embodiments, a modified oligonucleotide is attached to the first functional group during synthesis, and a conjugate moiety is attached to a second functional group during synthesis. Then, the two compounds are mixed under specific conditions to yield the final oligomeric compound. In certain embodiments, the conjugate moiety comprises two peptide loops attached to a molecular scaffold. Such reactions that are compatible with both oligonucleotide and peptide chemistry have been previously described and are often called “bioconjugation” reactions. These reactions include strain promoted azido-alkyne cycloaddition (SPAAC), copper-catalyzed click reaction (CuAAC), active ester conjugation to an amino modified oligonucleotide, maleimide-thiol Michael addition, ketol/hydroxylamine ligation, the Staudinger ligation, reductive amination, thio ether formation, disulfide formation, reductive alkylation, catalyst-free N-arylation, sulfur fluoride exchange click reaction (SuFEx), and inverse demand Diels Alder reaction. Certain such reactions are described in, e.g., Jbara, et al., “Oligonucleotide Bioconjugation with Bifunctional Palladium Reagents”, Angew. Chem. Int. Ed. 2021, 60(21)12109-12115; Dong, et al., “Sulfur(VI) Fluoride Exchange (SuFEx): Another Good Reaction for Click Chemistry,” Angew. Chem. Int. Ed. 2014, 53(36):9430-9448.4; Zhang, et al., “Arylation Chemistry for Bioconjugation,” Angew. Chem. Int. Ed. Engl. 2019; 58(15): 4810-4839; Walsh, et al., “Site-selective modification strategies in antibody-drug conjugates” Chem. Soc. Rev., 2021, 50: 1305-1353; Tiefenbrunn, et al., “Chemoselective ligation techniques: modern applications of time-honored chemistry”, Biopolymers, 2010, 94(1):95-106; Drake, et al., Bioconjug. Chem. 2014, 25(7):1331-1341; Bode, Acc. Chem. Res., 2017, 50, 9, 2104-2115; J. Magano, B. Bock, et al, Org. Proc. Res. Dev. 2014, 18:142-151; Craig S. McKay and M. G. Finn, “Click Chemistry in Complex Mixtures: Bioorthogonal Bioconjugation”, Chemistry & Biology 2014; Mitchell P. Christy et al., Org. Lett. 2020, 22: 2365; Ren et al., Angew. Chem. Int. Ed. Engl. 2009, 48, 9658-9662; Rohrbacher, F. et al., Helv. Chim. Acta. 2018, 101; Baalmaan, et al, “A Bioorthogonal Click Chemistry Toolbox for Targeted Synthesis of Branched and Well-Defined Protein-Protein Conjugates”, Angew. Chem. Int. Ed. 2020 (59): 12885-12893; Lang, et al, “Biorthogonal Reactions for Labeling Proteins”, J. Am. Chem. Soc, 2014, 9(1):16-20; Nair, et al., “The Thiol-Michael Addition Click Reaction: A Powerful and Widely Used Tool in Materials Chemistry”, Chem. Mater. 2013 26(1):724-744; Kalia and Raines, “Hydrolytic Stability of Hydrazones and Oximes”, Angew. Chem. Int. Ed., 2008, 47:7523-7526.

Examples of conjugate linkers include but are not limited to pyrrolidine, 8-amino-3,6-dioxaoctanoic acid (ADO), succinimidyl 4-(N-maleimidomethyl) cyclohexane-1-carboxylate (SMCC) and 6-aminohexanoic acid (AHEX or AHA). Other conjugate linkers include but are not limited to substituted or unsubstituted C₁-C₁₀ alkyl, substituted or unsubstituted C₂-C₁₀ alkenyl or substituted or unsubstituted C₂-C₁₀ alkynyl, wherein a nonlimiting list of preferred substituent groups includes hydroxyl, amino, alkoxy, carboxy, benzyl, phenyl, nitro, thiol, thioalkoxy, halogen, alkyl, aryl, alkenyl and alkynyl.

In certain embodiments, conjugate linkers comprise 1-5 linker-nucleosides. In certain embodiments, conjugate linkers comprise 2-5 linker-nucleosides. In certain embodiments, conjugate linkers comprise exactly 3 linker-nucleosides. In certain embodiments, conjugate linkers comprise the TCA motif. In certain embodiments, such linker-nucleosides are modified nucleosides. In certain embodiments such linker-nucleosides comprise a modified sugar moiety. In certain embodiments, linker-nucleosides are unmodified. In certain embodiments, linker-nucleosides comprise an optionally protected heterocyclic base selected from a purine, substituted purine, pyrimidine or substituted pyrimidine. In certain embodiments, a cleavable moiety is a nucleoside selected from uracil, thymine, cytosine, 4-N-benzoylcytosine, 5-methyl cytosine, 4-N-benzoyl-5-methyl cytosine, adenine, 6-N-benzoyladenine, guanine and 2-N-isobutyrylguanine. It is typically desirable for linker-nucleosides to be cleaved from the oligomeric compound after it reaches a target tissue. Accordingly, linker-nucleosides are typically linked to one another and to the remainder of the oligomeric compound through cleavable bonds. In certain embodiments, such cleavable bonds are phosphodiester bonds.

Herein, linker-nucleosides are not considered to be part of the oligonucleotide. Accordingly, in embodiments in which an oligomeric compound comprises an oligonucleotide consisting of a specified number or range of linked nucleosides and/or a specified percent complementarity to a reference nucleic acid and the oligomeric compound also comprises a conjugate linker comprising linker-nucleosides, those linker-nucleosides are not counted toward the length of the oligonucleotide and are not used in determining the percent complementarity of the oligonucleotide for the reference nucleic acid. For example, an oligomeric compound may comprise (1) an oligonucleotide consisting of 18-30 nucleosides and (2) a conjugate linker comprising 1-10 linker-nucleosides that are contiguous with the nucleosides of the oligonucleotide. The total number of contiguous linked nucleosides in such an oligomeric compound is more than 30. Alternatively, an oligomeric compound may comprise an oligonucleotide consisting of 18-30 nucleosides and no conjugate linker. The total number of contiguous linked nucleosides in such an oligomeric compound is no more than 30. Unless otherwise indicated conjugate linkers comprise no more than 10 linker-nucleosides. In certain embodiments, conjugate linkers comprise no more than 5 linker-nucleosides. In certain embodiments, conjugate linkers comprise no more than 3 linker-nucleosides. In certain embodiments, conjugate linkers comprise no more than 2 linker-nucleosides. In certain embodiments, conjugate linkers comprise no more than 1 linker-nucleoside.

In certain embodiments, it is desirable for a conjugate moiety to be cleaved from the oligonucleotide. For example, in certain circumstances oligomeric compounds comprising a particular conjugate moiety are better taken up by a particular cell type, but once the oligomeric compound has been taken up, it is desirable that the conjugate moiety be cleaved to release the unconjugated or parent oligonucleotide. Thus, certain conjugate linkers may comprise one or more cleavable moieties. In certain embodiments, a cleavable moiety is a cleavable bond. In certain embodiments, a cleavable moiety is a group of atoms comprising at least one cleavable bond. In certain embodiments, a cleavable moiety comprises a group of atoms having one, two, three, four, or more than four cleavable bonds. In certain embodiments, a cleavable moiety is selectively cleaved inside a cell or subcellular compartment, such as a lysosome. In certain embodiments, a cleavable moiety is selectively cleaved by endogenous enzymes, such as nucleases.

In certain embodiments, a cleavable bond is selected from among: an amide, an ester, an ether, one or both esters of a phosphodiester, a phosphate ester, a carbamate, or a disulfide. In certain embodiments, a cleavable bond is one or both of the esters of a phosphodiester. In certain embodiments, a cleavable moiety comprises a phosphate or phosphodiester. In certain embodiments, the cleavable moiety is a phosphodiester linkage between an oligonucleotide and a conjugate moiety.

In certain embodiments, a cleavable moiety comprises or consists of one or more linker-nucleosides. In certain such embodiments, the one or more linker-nucleosides are linked to one another and/or to the remainder of the oligomeric compound through cleavable bonds. In certain embodiments, such cleavable bonds are unmodified phosphodiester bonds. In certain embodiments, a cleavable moiety is 2′-deoxy nucleoside that is attached to either the 3′ or 5′-terminal nucleoside of an oligonucleotide by a phosphate internucleoside linkage and covalently attached to the remainder of the conjugate linker or conjugate moiety by a phosphate or phosphorothioate linkage. In certain such embodiments, the cleavable moiety is 2′-deoxyadenosine.

In certain embodiments, oligomeric compounds described herein comprise an oligonucleotide linked to a conjugate moiety by a conjugate linker, wherein the oligomeric compound is prepared using Click chemistry known in the art. Compounds have been prepared using Click chemistry wherein alkynyl phosphonate internucleoside linkages on an oligomeric compound attached to a solid support are converted into the 1,2,3-triazolylphosphonate internucleoside linkages and then cleaved from the solid support (Krishna et al., J Am. Chem. Soc. 2012, 134(28), 11618-11631), which is incorporated by reference herein in its entirety. Additional conjugate linkers suitable for use in several embodiments are prepared by Click chemistry described in “Click Chemistry for Biotechnology and Materials Science” Ed. Joerg Laham, Wiley 2009, which is incorporated by reference herein in its entirety.

In certain embodiments, compounds comprise an oligonucleotide, a cell-targeting moiety, and a conjugate linker. In certain embodiments, oligomeric compounds comprise an oligonucleotide, a hepatic asialoglycoprotein receptor (ASGP-R) ligand, and a conjugate linker. In certain embodiments, oligomeric compounds comprise an oligonucleotide, a N-acetyl galactosamine (GalNAc) ligand, and a conjugate linker. In certain embodiments, oligomeric compounds comprise an oligonucleotide, a GalNAc trimer, a branching group, a conjugate linker, and optionally modifications to the GalNAc ligands. In certain embodiments, oligomeric compounds comprise an oligonucleotide, two or more GalNAc ligands, a branching group, a conjugate linker, and optionally modifications to the GalNAc ligands. In certain embodiments, a conjugate linker connects GalNAc ligand to an oligonucleotide.

In certain embodiments, two or more GalNAc ligands are covalently connected to a conjugate linker, and the conjugate linker is covalently connected to the 3′ end of an oligonucleotide. In certain embodiments, a three GalNAc cluster is covalently connected to a conjugate linker, and the conjugate linker is covalently connected to the 3′ end of an oligonucleotide. In certain embodiments, two or more GalNAc ligands are covalently connected to a conjugate linker, and the conjugate linker is covalently connected to the 5′ end of an oligonucleotide. In certain embodiments, a three GalNAc cluster is covalently connected to a conjugate linker, and the conjugate linker is covalently connected to the 5′ end of an oligonucleotide. In certain embodiments, two or more GalNAc ligands are covalently connected to a conjugate linker, and the conjugate linker is covalently connected to an internal position of an oligonucleotide. In certain embodiments, a three GalNAc cluster is covalently connected to a conjugate linker, and the conjugate linker is covalently connected to an internal position of an oligonucleotide. In certain embodiments, an internal position of an oligonucleotide is a 2′-position of a modified sugar moiety of a nucleoside within the internal region of an oligonucleotide that is not the 5′ terminal nucleoside or the 3′ terminal nucleoside. In certain embodiments, an internal position of an oligonucleotide is a modified internucleoside linkage of the oligonucleotide.

In certain embodiments, a sense oligomeric compound is conjugated to a THA-GalNAc conjugate group attached to the 5′-OH of the oligonucleotide. The structure of THA-GalNAc is:

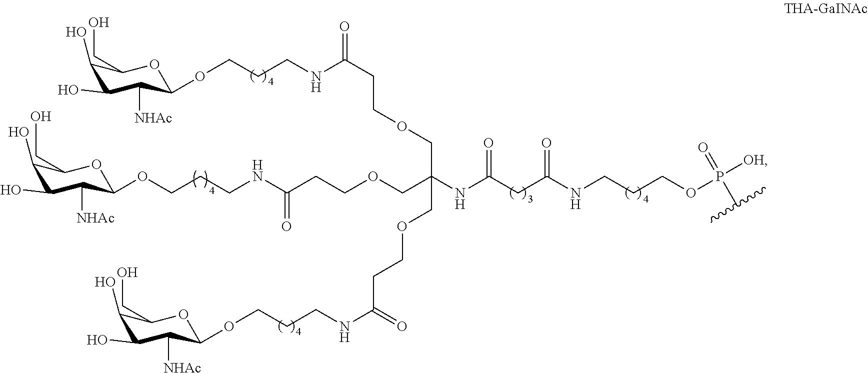

In certain embodiments a sense oligomeric compound is conjugated to a HPPO-GalNAc conjugate group attached to the 3′-OH of the oligonucleotide. The structure of HPPO-GalNAc is:

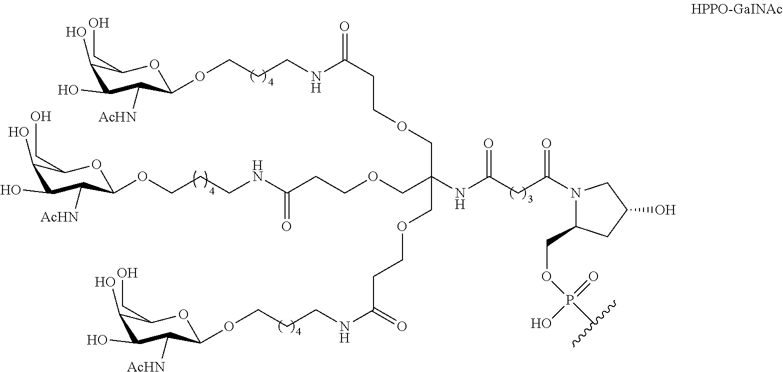

### B. Certain Terminal Groups

As used herein, “terminal group” means a chemical group or group of atoms that is covalently linked to a terminus of an oligonucleotide. Examples of a terminal group include, but are not limited to, a conjugate group, a capping group, a phosphate moiety, a protecting group, a modified or unmodified nucleoside, and two or more nucleosides that are independently modified or unmodified, wherein one or more groups is attached to either or both ends of an oligonucleotide. In certain embodiments, one or more terminal groups is attached to either or both ends of an oligonucleotide. In certain embodiments, one or more terminal groups is attached at the 3′ and/or 5′-end of the oligonucleotide. In certain embodiments, one or more terminal groups is attached at the 3′-end of the oligonucleotide. In certain embodiments, one or more terminal groups is attached at the 5′-end of the oligonucleotide. In certain embodiments, one or more terminal groups is attached at the 3′-end of the oligonucleotide and one or more terminal groups is attached at the 5′-end of the oligonucleotide. In certain embodiments, a terminal group is attached at the 3′ and/or 5′-end of the oligonucleotide. In certain embodiments, a terminal group is attached at the 3′-end of the oligonucleotide. In certain embodiments, a terminal group is attached near the 3′-end of the oligonucleotide. In certain embodiments, a terminal group is attached at the 5′-end of the oligonucleotide. In certain embodiments, a terminal group is attached near the 5′-end of the oligonucleotide. In certain embodiments, a terminal group is attached at the 3′-end of the oligonucleotide and a terminal group is attached at the 5′-end of the oligonucleotide.

In certain embodiments, an oligomeric compound comprises one or more terminal groups. In certain embodiments, an oligomeric compound comprises a terminal group comprising a stabilized 5′-phosphate. Stabilized 5′-phosphates include, but are not limited to 5′-phosphonates, including, but not limited to 5′-vinylphosphonate, 5′-methylphosphonate. In certain embodiments, a terminal group comprises one or more abasic sugar moieties. In certain embodiments, a terminal group comprises one or more inverted sugar moieties and/or inverted nucleosides. In certain embodiments, a terminal group comprises one or more 2′-linked nucleosides or sugar moieties. In certain embodiments, the 2′-linked terminal group is an abasic sugar moiety. In certain embodiments, an antisense oligomeric compound comprises a vinylphosphonate. In certain embodiments, each antisense oligomeric compound has a vinyl phosphonate moiety on the 5′-end (5′-VP).

### IV. Target Nucleic Acids

### A. APOC3

In certain embodiments, oligomeric compounds comprise an oligonucleotide comprising a region that is complementary to a target nucleic acid, wherein the target nucleic acid is APOC3. In certain embodiments, APOC3 nucleic acid has the sequence set forth in SEQ ID NO: 1 (GENBANK Accession No. NM_000040.2). In certain embodiments, contacting a cell with an oligomeric compound complementary to SEQ ID NO: 1 reduces the amount of APOC3 RNA, and in certain embodiments reduces the amount of APOCIII protein. In certain embodiments, the oligomeric compound comprises of a modified oligonucleotide. In certain embodiments, the oligomeric compound comprises a modified oligonucleotide and a conjugate group. In certain embodiments, the oligomeric compound comprises a modified oligonucleotide and one or more terminal group(s). In certain embodiments, the oligomeric compound comprises a modified oligonucleotide and a conjugate group and one or more terminal group(s).

In certain embodiments, oligomeric agents comprise an antisense oligomeric compound comprising a region that is complementary to a nucleobase sequence of a target APOC3 nucleic acid. In certain embodiments, oligomeric agents comprise an antisense oligomeric compound comprising a region that is complementary to a nucleobase sequence of a target APOC3 nucleic acid, and a sense oligomeric compound comprising a region that is complementary to the nucleobase sequence of the antisense oligomeric compound. In certain embodiments, the target nucleic acid is an endogenous APOC3 RNA molecule. In certain embodiments, the target APOC3 nucleic acid encodes APOCIII protein. In certain such embodiments, the target APOC3 nucleic acid is selected from: a mature mRNA, including exonic and untranslated regions. In certain embodiments, the oligomeric agent or oligomeric duplex is an RNAi agent. In certain embodiments, antisense agents comprise an antisense oligomeric compound comprising a region that is complementary to a nucleobase sequence of a target APOC3 nucleic acid.

In certain embodiments, antisense oligomeric compounds are complementary to a nucleobase sequence in a target APOC3 nucleic acid over the entire length of the modified oligonucleotide. In certain embodiments, antisense oligomeric compounds are 99%, 95%, 90%, 85%, or 80% complementary to an equal length portion of the target APOC3 nucleic acid. In certain embodiments, antisense oligomeric compounds are at least 80% complementary to a nucleobase sequence in the target APOC3 nucleic acid over the entire length of the oligonucleotide and comprise a region that is 100% or fully complementary to a sequence in the target APOC3 nucleic acid.

In certain embodiments, a region of full complementarity is from 6 to 20, 10 to 18, 14 to 18, 16 to 20, or 18 to 20 nucleobases in length. In certain embodiments, the complementary region comprises or consists of at least 8, at least 9, at least 10, at least 11, at least 12, at least 13, at least 14, at least 15, at least 16, at least 17, at least 18, at least 19, at least 20, at least 21, at least 22, or at least 23 contiguous nucleobases. In certain embodiments, the complementary region comprises or consists of at least 8, at least 9, at least 10, at least 11, at least 12, at least 13, at least 14, at least 15, at least 16, at least 17, at least 18, at least 19, at least 20, at least 21, at least 22, or 23 contiguous nucleobases. In certain embodiments, the complementary region constitutes 75%, 80%, 85%, 90%, or 95% of the nucleosides of the antisense oligomeric compound. In certain embodiments, the complementary region constitutes all of the nucleosides of the antisense oligomeric compound. In certain embodiments, the complementary region of the antisense oligomeric compound is at least 99%, 95%, 90%, 85%, or 80% complementary to a nucleobase sequence in the target APOC3 nucleic acid. In certain embodiments, the complementary region of the antisense oligomeric compound is 100% complementary to a nucleobase sequence in the target APOC3 nucleic acid.

In certain embodiments, antisense oligomeric compounds comprise one or more mismatched nucleobases relative to the target APOC3 nucleic acid. In certain embodiments, antisense activity against the target is reduced by such mismatch, but activity against a non-target is reduced by a greater amount. Thus, in certain embodiments selectivity of the antisense oligomeric compounds is improved. In certain embodiments, antisense oligomeric compounds are at least 80% complementary to a nucleobase sequence in the target APOC3 nucleic acid over the entire length of the oligonucleotide and comprise no more than one to three mismatches with target APOC3 nucleic acid. In certain embodiments, antisense oligomeric compounds are at least 80% complementary to a nucleobase sequence in the target APOC3 nucleic acid over the entire length of the oligonucleotide and comprise no more than one to three mismatches with target nucleic acid, not inclusive of terminal nucleobases of the antisense oligomeric compound. In certain embodiments, a mismatch is specifically positioned within an antisense oligomeric compound. In certain embodiments, a mismatch is at position 3, 4, 5, 6, 7, 8, 9, 10, 11, or 12 from the 5′-end of the antisense oligomeric compound. In certain embodiments, a mismatch is at position 11, 10, 9, 8, 7, 6, 5, 4, 3, or 2 from the 3′-end of the antisense oligomeric compound. In certain embodiments, a mismatch is at position 1, 2, 3, or 4 from the 5′-end of the antisense oligomeric compound. In certain embodiments, a mismatch is at position 4, 3, 2, or 1 from the 3′-end of the antisense oligomeric compound.

### B. Target Nucleic Acids in Certain Tissues

In certain embodiments, oligomeric compounds comprise an oligonucleotide comprising a region that is complementary to a nucleobase sequence in a APOC3 target nucleic acid, wherein the APOC3 target nucleic acid is expressed in a pharmacologically relevant tissue. In certain embodiments, the APOC3 target nucleic acid is expressed in the liver cells and hepatic tissues.

### C. Oligonucleotide Sequences

Provided herein are oligomeric compounds comprising modified oligonucleotides complementary to a sequence of nucleobases in a APOC3 nucleic acid, such as, for example, a human APOC3 nucleic acid, such as SEQ ID NO: 1 (GENBANK Accession No. NM_000040.2), or SEQ ID NOS; 2-5 and compositions comprising such oligomeric compounds. In certain embodiments, a modified oligonucleotide has a nucleobase sequence comprising or consisting of a nucleobase sequence that is at least 80%, at least 85%, at least 90%, at least 95%, or at least 99% complementary to a sequence of nucleobases in SEQ ID NO: 1 or SEQ ID NOS; 2-5. In certain embodiments, a modified oligonucleotide has a nucleobase sequence comprising or consisting of a nucleobase sequence that is at least 80%, at least 85%, at least 90%, at least 95%, or at least 99% complementary to a nucleobase sequence comprising or consisting of a nucleobase sequence that is at least 80%, at least 85%, at least 90%, at least 95%, or at least 99% complementary to a sequence of nucleobases in SEQ ID NO: 1 or SEQ ID NOS; 2-5. In certain embodiments, a modified oligonucleotide has a nucleobase sequence comprising or consisting of a nucleobase sequence that is 100% complementary to a sequence of nucleobases in SEQ ID NO: 1 or SEQ ID NOS; 2-5. In certain embodiments, a modified oligonucleotide has a nucleobase sequence comprising or consisting of a nucleobase sequence that is at least 80%, at least 85%, at least 90%, at least 95%, or at least 99% complementary to a nucleobase sequence comprising or consisting of a nucleobase sequence that is 100% complementary to a sequence of nucleobases in SEQ ID NO: 1 or SEQ ID NOS; 2-5. In certain embodiments, a modified oligonucleotide has a nucleobase sequence comprising or consisting of a nucleobase sequence that is selected from among the sequences provided in SEQ ID NOS: 2-5, or SEQ ID NOS: 6-8.

### V. Methods and Uses

### A. Antisense Activity

In certain embodiments, oligomeric compounds are capable of hybridizing to a target nucleic acid, resulting in at least one antisense activity; such oligomeric compounds and oligomeric duplexes are antisense agents.

In certain antisense activities, hybridization of an antisense oligomeric compound to a target nucleic acid results in recruitment of a protein that cleaves the target nucleic acid. For example, in certain antisense activities, an antisense agent or a portion of an antisense agent is loaded into an RNA-induced silencing complex (RISC), ultimately resulting in cleavage of the target nucleic acid. For example, certain antisense agents result in cleavage of the target nucleic acid by Argonaute. Antisense agents that are loaded into RISC are RNAi agents. RNAi agents may be double-stranded (siRNA or dsRNAi) or single-stranded (ssRNA). In certain embodiments, RNAi agents are capable of RISC-mediated modulation of a target nucleic acid in a cell. In certain embodiments, such compounds reduce or inhibit the amount or activity of a target nucleic acid by 25% or more in the standard in vitro assay. In certain embodiments, RNAi agents selectively affect one or more target nucleic acid. Such RNAi agents comprise a nucleobase sequence that hybridizes to one or more target nucleic acid, resulting in one or more desired antisense activity. In certain embodiments, an RNAi agent does not hybridize to one or more non-target nucleic acid or does not hybridize to one or more non-target nucleic acid in such a way that results in significant undesired antisense activity.

Antisense activities may be observed directly or indirectly. In certain embodiments, observation or detection of an antisense activity involves observation or detection of a change in an amount of a target nucleic acid or protein encoded by such target nucleic acid, a change in the ratio of splice variants of a nucleic acid or protein and/or a phenotypic change in a cell or animal.

### B. Treatment, Prophylaxis

### Overview of Familial Chylomicronemia Syndrome

Familial Chylomicronemia Syndrome (FCS) is an inherited disease characterized by severe hypertriglyceridemia and chylomicronemia. It is a rare autosomal recessive disease that can be diagnosed either in childhood or adulthood. FCS is characterized by frequent and severe abdominal pain, repetitive colicky pain, repeated episodes of potentially fatal acute pancreatitis, and in children, can result in a failure to thrive (Brunzell J D. Familial Lipoprotein Lipase Deficiency. In GeneReviews edited by Adam M P Pagon R A, Bird T D, et al. 1999-2011. Seattle, W A: University of Washington, Seattle; Tremblay K, Methot J, Brisson D, et al. J Clin Lipidol 2011; 5: 37-44). Physical examination frequently reveals eruptive xanthomas, lipemia retinalis and hepatosplenomegaly, and plasma from patients appears lactescent, interfering with determination of other laboratory parameters. Fasting plasma TG levels in FCS patients are typically 10-fold to 100-fold above normal (1,500 to 15,000 mg/dL), despite extreme dietary fat restriction (20 g or approximately 15-20% of daily calorie intake. Patients with FCS often present in infancy or childhood with recurrent episodes of abdominal pain or pancreatitis, eruptive xanthomas or hepatomegaly. The diagnosis of FCS is then established by genotyping or confirmation of very low or absent lipoprotein lipase (LPL) enzyme activity in post-heparin plasma. Patients with FCS carry a heavy burden of medical complications, the most serious being an extreme risk of recurrent and potentially fatal pancreatitis. Due to the recurrent episodes of acute pancreatitis, these patients may also develop chronic pancreatitis and signs of exocrine or endocrine pancreatic insufficiency, including diabetes mellitus (Gaudet D, Methot J, Dery S, et al. Gene Ther 2013; 20: 361-369). While the pathophysiology underlying chylomicron-related pancreatitis has not been completely elucidated, one hypothesis is that large chylomicrons lodged in pancreatic capillaries are exposed to pancreatic lipase, resulting in release of free fatty acids through the hydrolysis of chylomicron-associated TGs. High concentrations of free fatty acids are thought to damage pancreatic cells leading to emergent pancreatitis (Yang F, Wang Y, Sternfeld L, et al. Acta Physiol (Oxf) 2009; 195: 13-28.; Berglund L, Brunzell J D, Goldberg A C, et al. J Clin Endocrinol Metab 2012; 97: 2969-2989). FCS significantly affects patients' health related quality of life (HRQoL). Bloating, generalized abdominal pain, asthenia, anxiety about potential painful attacks and overall health, difficulty concentrating and “brain fog” are commonly reported symptoms of FCS. The psychosocial burden of FCS is also increased by dietary fat restriction and overall interference with social interactions and ability to work (Davidson M, Stevenson M, Hsieh A, et al. Expert Rev Cardiovasc Ther 2017; 15: 415-423; Gelrud A, Williams K R, Hsieh A, et al. Expert Rev Cardiovasc Ther 2017; 15: 879-887; Davidson M, Stevenson M, Hsieh A, et al. J Clin Lipidol 2018; 12: 898-907.e892; Fox R S, Peipert J D, Llonch M V, et al. Expert Rev Cardiovasc Ther 2020: 1-8). The etiology of extreme hypertriglyceridemia in FCS is considered to be ineffective TG clearance, due to an extremely low level of LPL activity. LPL normally functions to hydrolyze TGs in chylomicrons along the luminal surface of capillaries, mainly in heart, skeletal muscle, and adipose tissue, promoting TG clearance from the circulation. LPL is regulated by a number of key genes, and loss-of-function mutations in one of these genes, or the LPL gene itself, results in FCS (Surendran R P, Visser M E, Heemelaar S, et al. J Intern Med 2012; 272: 185-196). In addition to loss of function mutations, null mutations, and nonsense mutations in the LPL gene, other genes currently identified in FCS patients, and known to directly influence LPL activity include: apolipoprotein C-II (APOC2) a cofactor for LPL (Schuster K B, Wilfert W, Evans D, et al. Clin Chim Acta 2011; 412: 240-244); apolipoprotein A-V (APOA5) (Schaap F G, Rensen P C, Voshol P J, et al. J Biol Chem 2004; 279: 27941-27947); lipase maturation Factor 1 (LMF1), a transmembrane protein involved in LPL maturation (Doolittle M H, Neher S B, Ben-Zeev O, et al J Biol Chem 2009; 284: 33623-33633), glycosylphosphotidylinositol-anchored HDL-binding protein 1 (GP1HBP1), a capillary endothelial cell protein that provides a platform for LPL-mediated processing of chylomicrons (Beigneux A P, Davies B S, Gin P, et al. Cell Metab 2007; 5: 279-291).

### Overview of Familial Partial Lipodystrophy

Familial Partial Lipodystrohpy refers to a familial disorder characterized by selective, progressive loss of body fat (adipose tissue) from various areas of the body. Individuals with FPL often have reduced subcutaneous fat in the arms and legs, and the head and trunk regions may or may not have loss of fat. Conversely, affected individuals may also have excess subcutaneous fat accumulation in other areas of the body, especially the neck, face and intra-abdominal regions. In many cases, adipose tissue loss begins during puberty. FPL can be associated with a variety of metabolic abnormalities. FPL is associated with certain metabolic complications. These complications can include an inability to metabolize glucose, elevated levels of triglycerides, and diabetes. Six different subtypes of FPL have been identified. Each subtype is caused by a mutation in a different gene. Four forms of FPL are inherited as autosomal dominant traits; one form is inherited as an autosomal recessive trait. The mode of inheritance of FPL, Kobberling variety is unknown. Types of FPL include FPL2 (Dunnigan variety), FPL1 (Kobberling variety), FPL3 (PPARG Mutations), FPL4 (PLIN1 Mutations), FPL5 (AKT2 Mutations), and Autosomal Recessive FPL (Type 6, CIDEC mutation).

### Overview of Severe Hypertriglyceridemia

As used herein, Severe Hypertriglyceridemia (SHTG) refers to a condition in which a subject has triglycerides at a level at which chylomicrons appear in the blood. In certain embodiments, a subject has at triglycerides of at least 500 mg/dL. SHTG may be acquired or familial. For example, a subject having FCS or FPL may also be diagnosed as having SHTG. In certain embodiments, the subject has triglycerides of at least 880 mg/dL. In certain embodiments, the subject has triglycerides of at least 1000 mg/dL. SHTG may arise in subjects having obesity, a history of alcohol abuse, and/or diabetes. SHTG may arise due to a combination of weak genetic factors combined with secondary factors such as certain medications (e.g., oral estrogens, glycocorticosteroids, protease inhibitors, some antihypertensive medications such as hydrochlorothiazide, and nonselective beta-blockers, retinoic acid (isotretinoin), tamoxifen, raloxifen, cyclosporin, sirolimus, bile acid-binding resins, and antipsychotic medications including clozapine and olanzapine) or metabolic disorders (e.g., obesity, diabetes, hypothyroidism, or kidney disease), or from genetic factors alone. Patients having SHTG are at risk for acute pancreatitis. See, e.g., Cybulska, B. et al., Kardiologia Polska 2013; 71, 10:1007-1012.

In certain embodiments, methods described herein are sufficiently effective to ameliorate at least one symptom of FCS in a human subject. In certain embodiments, the at least one symptom is severe elevations in chylomicrons. In certain embodiments, the at least one symptom is extremely elevated in SHTG levels (always reaching well above 1000 mg/dL and not infrequently rising as high as 10,000 mg/dL or more). In certain embodiments, the at least one symptom is episodes of abdominal pain. In certain embodiments, the at least one symptom is recurrent acute pancreatitis. In certain embodiments, the at least one symptom is repetitive colicky pain. In certain embodiments, the at least one symptom is eruptive xanthomas. In certain embodiments, the at least one symptom is hepatosplenomegaly. In certain embodiments, the at least one symptom is physical fatigue. In certain embodiments, the at least one symptom is difficulty thinking. In certain embodiments, the at least one symptom is diarrhea. In certain embodiments, the at least one symptom is difficulty thinking. In certain embodiments, the at least one symptom is recurrent acute pancreatitis. In certain embodiments, the at least one symptom is lipemia retinalis. In certain embodiments, the at least one symptom is a combination of any one of severe chylomicronemia, severe hypertriglyceridemia, frequent and severe abdominal pain, repetitive colicky pain, physical fatigue, difficulty thinking, diarrhea, recurrent acute pancreatitis, eruptive xanthomas, lipemia retinalis, and hepatosplenomegaly.

In certain embodiments, methods described herein are sufficiently effective to ameliorate any one of severe chylomicronemia, severe hypertriglyceridemia, frequent and severe abdominal pain, repetitive colicky pain, physical fatigue, difficulty thinking, diarrhea, recurrent acute pancreatitis, eruptive xanthomas, lipemia retinalis, and hepatosplenomegaly. In certain embodiments, methods described herein are sufficiently effective to ameliorate at least one symptom of FCS in a human subject as assessed by a clinically relevant test, score or scale. In certain embodiments, the clinically relevant scale is the Patient Global Impression of Severity (PGIS) Scale. In certain embodiments, the clinically relevant scale is the Patient Global Impression of Change (PGIC) Scale. In certain embodiments, the clinically relevant test is fasting triglyceride levels. In certain embodiments, the clinically relevant test is fasting apoB-48 levels. In certain embodiments, the clinically relevant test, score or scale is a decrease the adjudicated pancreatitis event rate in patients with >2 events in 5 years prior to enrollment. In certain embodiments, the clinically relevant test, score or scale is number of emergency room (ER) visits, incidence of all-cause hospitalizations, and total inpatient days. In certain embodiments, health-related quality of life is measured by the PROMIS 29+2 Profile vs. 2.1 questionnaire.

In certain embodiments, provided herein are methods of decreasing, reducing and/or inhibiting APOC3 expression, APOC3 RNA levels and/or ApoCIII levels and/or activity, in a subject having, or at risk of having, a disease, disorder, condition or injury associated with APOC3 and/or ApoCIII, such as a disease, disorder, condition or injury associated with lipoprotein metabolism misregulation, wherein the method includes administering to the subject an oligomeric agent, antisense oligomeric compound, or oligomeric duplex comprising or consisting of a modified oligonucleotide having a nucleobase sequence complementary to a nucleobase sequence in a APOC3 nucleic acid, thereby inhibiting or reducing expression of APOC3 nucleic acid in the subject. In certain embodiments, administering such oligomeric agent or oligomeric duplex reduces and/or inhibits APOC3 expression, APOC3 RNA levels and/or ApoCIII levels and/or activity in the plasma/serum blood or liver of the subject. In certain embodiments, administering such oligomeric agent or oligomeric duplex reduces and/or inhibits APOC3 expression, APOC3 RNA levels and/or ApoCIII levels and/or activity in the liver and/or blood, of the subject. In some instances, such oligomeric agent or oligomeric duplex is administered parenterally. In some instances, an oligomeric agent or oligomeric duplex is administered intravenously or subcutaneously. In certain embodiments, the detectable amount of the APOC3 RNA may be reduced by at least 10%, at least 20%, at least 30%, at least 40%, at least 50%, at least 60%, at least 70%, at least 80%, or at least 90%. In certain embodiments, an oligomeric agent comprising or consisting of, or an oligomeric duplex comprising, an antisense oligomeric compound comprising a nucleobase sequence complementary to a nucleobase sequence in SEQ ID NO: 1 is capable of decreasing or reducing a detectable amount of a ApoCIII protein in a cell, organ or tissue, e.g., the liver of the subject, when the compound is administered to the cell, a tissue, and/or subject. In certain embodiments, the detectable amount of the ApoCIII protein may be reduced by at least 10%, at least 20%, at least 30%, at least 40%, at least 50%, at least 60%, at least 70%, at least 80%, or at least 90%.

In certain embodiments, provided herein are methods for preventing, treating, or delaying or preventing the development or progression of, diseases, disorders, conditions or injuries associated with APOC3 and/or ApoCIII, such as a disease, disorder, condition or injury associated with lipoprotein metabolism misregulation, wherein the method comprises administering to a subject an oligomeric agent or oligomeric duplex (e.g., an antisense oligomeric compound, an antisense agent) having a nucleobase sequence complementary to a nucleobase sequence in a APOC3 nucleic acid wherein a first oligomeric compound comprises a modified oligonucleotide consisting of 18 to 50 linked nucleosides, wherein the nucleobase sequence of the first modified oligonucleotide comprises at least 18, at least 19, at least 20, at least 21, at least 22, or at least 23 contiguous nucleobases of the nucleobase sequence of any one of SEQ ID NOs: 2-5.

Provided are methods of ameliorating, preventing, or delaying the onset of, one or more symptoms associated with diseases, disorders, conditions or injuries associated with APOC3 or ApoCIII, such as hypertriglyceridemia, coronary heart disease, a cardiovascular disease, disorder, condition or injury associated with triglyceride misregulation, wherein the method comprises administering to a subject an oligomeric agent comprising or consisting of, or an oligomeric duplex comprising a modified antisense oligonucleotide (e.g., an antisense oligomeric compound) having a nucleobase sequence complementary to a nucleobase sequence in a APOC3 nucleic acid wherein a first oligomeric compound comprises a modified oligonucleotide consisting of 18 to 50 linked nucleosides, wherein the nucleobase sequence of the first modified oligonucleotide comprises at least 18, at least 19, at least 20, at least 21, at least 22, or at least 23 contiguous nucleobases of the nucleobase sequence of any one of SEQ ID NOs: 2-5. Examples of diseases, disorders or conditions associated with APOC3 and/or ApoCIII treatable with the compounds, compositions, and methods provided herein include a disease, disorder or condition associated with lipoprotein metabolism misregulation (e.g., hypertriglyceridemia, lipidemia, atherosclerotic cardiovascular/cardiac injury, disease, disorder or condition as further described herein). In certain embodiments, the disease, disorder, condition or injury is hypertriglyceridemia, non-familial hypertriglyceridemia, familial hypertriglyceridemia, heterozygous familial hypertriglyceridemia, homozygous familial hypertriglyceridemia, mixed dyslipidemia, atherosclerosis, a risk of developing atherosclerosis, coronary heart disease, a history of coronary heart disease, early onset coronary heart disease. In certain embodiments the disease, disorder or condition is hypertriglyceridemia (HTG) or atherosclerotic cardiovascular disease (ASCVD) or coronary artery disease (CAD). In certain embodiments the ASCVD is ischemic vascular disease (IVD), ischemic heart disease (IHD). In certain embodiments the hypertriglyceridemia is genetic hypertriglyceridemia. In certain embodiments the hypertriglyceridemia is familial chylomicronemia syndrome (FCS) or familial partial lipodystrophy (FPL). In certain embodiments, the subject has a cardiovascular and/or metabolic disease, disorder or condition. In certain embodiments the subject has one or more risk factors for coronary heart disease, type II diabetes, type II diabetes with dyslipidemia, dyslipidemia, hyperlipidemia, hypercholesterolemia, hyperfatty acidemia, hepatic steatosis, non-alcoholic steatohepatitis, pancreatitis and/or non-alcoholic fatty liver disease.

Additionally provided are methods of reducing expression of APOC3 or reducing ApoCIII protein in a cell comprising contacting the cell or tissue with an oligomeric agent comprising or consisting of, or an oligomeric duplex comprising a modified antisense oligonucleotide (e.g., an antisense oligomeric compound) having a nucleobase sequence complementary to sequence in a APOC3 nucleic acid. In certain embodiments, the cell is a liver cell, hepatocyte, or tissue.

Triglyceride dysfunction associated with lipid misregulation is associated with cardiovascular and cardiac diseases and disorders. Symptoms of a disease, disorder, condition or injury associated with triglyceride misregulation include symptom of a disease, disorder or condition associated with elevated triglycerides is episodes of abdominal pain, physical fatigue, difficulty thinking, diarrhea, recurrent acute pancreatitis, eruptive cutaneous xanthomata, and hepatosplenomegaly or a combination thereof. In certain embodiments, the disease, disorder or condition is hypertriglyceridemia or atherosclerotic cardiovascular disease (ASCVD) or coronary artery disease (CAD).

Thus, in certain embodiments, a method comprises administering to a subject an oligomeric agent comprising or consisting of an oligomeric duplex comprising an antisense oligomeric compound having a nucleobase sequence complementary to a sequence in a APOC3 nucleic acid. In certain embodiments, the subject has or is at risk for developing an atherosclerotic cardiovascular disease, condition or disorder, hypertriglyceridemia, cardiovascular disease, and/or coronary heart disease. In certain embodiments, provided herein are methods for preventing, treating, or delaying or preventing the development or progression of, an atherosclerotic cardiovascular disease, condition or disorder, hypertriglyceridemia, cardiovascular disease, and/or coronary heart disease, wherein the method comprises administering to a subject an oligomeric agent or oligomeric duplex (e.g., a modified antisense oligonucleotide, an antisense oligomeric compound, an antisense agent) having a nucleobase sequence complementary to a nucleobase sequence in a APOC3 nucleic acid wherein a first oligomeric compound comprises a modified oligonucleotide consisting of 18 to 50 linked nucleosides, wherein the nucleobase sequence of the first modified oligonucleotide comprises at least 18, at least 19, at least 20, at least 21, at least 22, or at least 23 contiguous nucleobases of the nucleobase sequence of any one of SEQ ID NOs: 2-5. In certain embodiments, the subject has or is at risk for developing hypertriglyceridemia, a cardiovascular disease, disorder, condition, a metabolic disease, disorder, or condition, or an inflammatory disease disorder or condition.

In certain embodiments, provided herein are methods for preventing, treating, or delaying or preventing the development or progression of a cardiovascular, metabolic, and/or inflammatory disease, disorder, condition, wherein the method comprises administering to a subject an oligomeric agent or oligomeric duplex (e.g., a modified antisense oligonucleotide, an antisense oligomeric compound, an antisense agent) having a nucleobase sequence complementary to a nucleobase sequence in a APOC3 nucleic acid wherein a first oligomeric compound comprises a modified oligonucleotide consisting of 18 to 50 linked nucleosides, wherein the nucleobase sequence of the first modified oligonucleotide comprises at least 18, at least 19, at least 20, at least 21, at least 22, or at least 23 contiguous nucleobases of the nucleobase sequence of any one of SEQ ID NOs: 2-5, In certain embodiments, the disease, disorder, condition or injury is a dyslipidemia, hypertriglyceridemia, atherosclerotic cardiovascular disease (ASCVD), and/or coronary artery disease (CAD). In certain embodiments, ASCVD is ischemic vascular disease (IVD). In certain embodiments, ASCVD is ischemic heart disease (IHD). In certain embodiments, metabolic disease disorder or condition is pancreatitis, diabetes, or insulin insensitivity. In certain embodiments, the hypertriglyceridemia is familial chylomicronemia syndrome (FCS) or familial partial lipodystrophy (FPL). In certain embodiments, hypertriglyceridemia is genetic hypertriglyceridemia. In certain embodiments the method prevents or protects against progression of coronary heart disease (CHD).

In certain embodiments, at least one symptom of the cardiovascular/hypertriglyceridemia, disease, condition, or disorder is ameliorated. In certain embodiments, the at least one symptom is selected from episodes of abdominal pain, physical fatigue, difficulty thinking, diarrhea, recurrent acute pancreatitis, eruptive cutaneous xanthomata, and hepatosplenomegaly or a combination thereof. In certain embodiments, administration of provided agents (e.g., an oligomeric agent, modified antisense oligonucleotide, an antisense oligomeric compound, or oligomeric duplex) to the subject reduces or delays the onset or progression of at least one of episodes of abdominal pain, physical fatigue, difficulty thinking, diarrhea, recurrent acute pancreatitis, eruptive cutaneous xanthomata, and hepatosplenomegaly or a combination thereof.

In certain embodiments, a method of inhibiting expression of APOC3 nucleic acid, such as RNA, in a subject having or at risk of a disease, injury, condition or disorder associated with APOC3 comprises administering to the subject an oligomeric agent, modified antisense oligonucleotide, an antisense oligomeric compound, or oligomeric duplex, any of which comprising a modified oligonucleotide having a nucleobase sequence complementary to a sequence of nucleobases in a APOC3 nucleic acid, thereby inhibiting expression of APOC3 nucleic acid in the subject. In certain embodiments, administering the oligomeric agent, modified oligonucleotide, or oligomeric duplex inhibits expression of APOC3 in the liver.

Certain embodiments are drawn to an oligomeric agent, modified oligonucleotide, or oligomeric duplex, any of which comprising an antisense oligomeric compound having a nucleobase sequence complementary to a nucleobase sequence in a APOC3 nucleic acid, for use in treating a disease, disorder, condition or injury associated with APOC3 and/or ApoCIII. In certain embodiments, the disease, disorder, condition or injury is associated with lipoprotein metabolism misregulation or postponing a symptom of a disease, disorder or condition associated with elevated triglycerides. Certain embodiments provided herein are drawn to an oligomeric agent, modified oligonucleotide, or oligomeric duplex, any of which comprising an antisense oligomeric compound having a nucleobase sequence complementary to a nucleobase sequence in a APOC3 nucleic acid, for use in treating a disease, disorder, condition or injury associated with lipoprotein metabolism misregulation, wherein the disease, disorder or condition wherein the disease, disorder or condition is hypertriglyceridemia or atherosclerotic cardiovascular disease (ASCVD) or coronary artery disease (CAD). In certain embodiments, the ASCVD is ischemic vascular disease (IVD). In certain embodiments, ASCVD is ischemic heart disease (IHD). In certain embodiments, the hypertriglyceridemia is genetic hypertriglyceridemia. In certain embodiments, the hypertriglyceridemia is familial chylomicronemia syndrome (FCS) or familial partial lipodystrophy (FPL).

Certain embodiments are drawn to an oligomeric agent, modified oligonucleotide, or oligomeric duplex, any of which comprising an antisense oligomeric compound having a nucleobase sequence complementary to a nucleobase sequence in a APOC3 nucleic acid, for the manufacture or preparation of a medicament for ameliorating, or delaying or preventing development or progression of a disease, disorder, condition or injury and/or for ameliorating, preventing or delaying the onset of one or more symptoms of a disease, disorder, condition or injury, wherein the disease, disorder, condition or injury is associated with or postponing a symptom of a disease, disorder or condition associated with elevated triglycerides. Certain embodiments provided herein are drawn to an oligomeric agent, modified oligonucleotide, or oligomeric duplex, any of which comprising a modified antisense oligonucleotide (an antisense oligomeric compound) having a nucleobase sequence complementary to a nucleobase sequence in a APOC3 nucleic acid, for the manufacture or preparation of a medicament for treatment of a disease, disorder, condition or injury associated with lipoprotein metabolism misregulation, wherein the disease, disorder or condition wherein the disease, disorder or condition is hypertriglyceridemia or atherosclerotic cardiovascular disease (ASCVD) or coronary artery disease (CAD). In certain embodiments, the ASCVD is ischemic vascular disease (IVD). In certain embodiments, ASCVD is ischemic heart disease (IHD). In certain embodiments, the hypertriglyceridemia is genetic hypertriglyceridemia. In certain embodiments, the hypertriglyceridemia is familial chylomicronemia syndrome (FCS) or familial partial lipodystrophy (FPL).

In certain embodiments, prophylactic administration of an oligomeric agent, modified antisense oligonucleotide, antisense oligomeric compound, or oligomeric duplex or composition provided herein to a subject at risk for a dyslipidemia, atherosclerotic cardiovascular disease (ASCVD), and/or coronary artery disease (CAD), is able to prevent, ameliorate, postpone or delay a symptom and/or development or progression of hypertriglyceridemia and/or ASCVD and/or CAD. In certain embodiments, an oligomeric agent, modified oligonucleotide, or oligomeric duplex is for the manufacture or preparation of a medicament for improving chylomicronemia, hypertriglyceridemia, abdominal pain, physical fatigue, difficulty thinking, diarrhea, acute pancreatitis, eruptive xanthomas, lipemia retinalis, or hepatosplenomegaly, or a combination of two or more of the foregoing in the subject.

In any of the methods or uses described herein, the oligomeric agent, modified oligonucleotide, oligomeric duplex or antisense agent can be any described herein.

In certain embodiments an oligomeric agent or oligomeric duplex has APOC3 RNA and/or protein reduction activity, and, in certain embodiments, cardiac APOC3 RNA and/or protein reduction activity, that is comparable to or greater than the APOC3 RNA and/or protein reduction activity of a comparator compound, e.g., having the same or similar nucleobase sequence and different modifications. In certain embodiments an oligomeric agent or oligomeric duplex has hepatic cell APOC3 RNA and/or protein reduction activity that is comparable to, or greater than, the hepatic cell APOC3 RNA and/or protein reduction activity of a comparator compound, e.g., having the same or similar nucleobase sequence and different modifications. Methods of detecting the level of and/or measuring the amount of APOC3 RNA and/or protein in a cell, organ, tissue, system or subject (e.g., animal) are described herein and/or known in the art. In certain embodiments, the amount of APOC3 RNA is reduced by at least 10%, at least 20%, at least 25%, at least 30%, at least 35%, at least 40%, at least 45%, at least 50%, at least 55%, at least 60%, at least 65%, at least 70%, at least 75%, at least 80%, at least 85%, or at least 90% in a cell (e.g., liver cell), organ (e.g., liver), tissue, system or subject (e.g., animal) that has been contacted with or administered an oligomeric agent or oligomeric duplex provided herein (or a composition comprising such oligomeric agent or oligomeric duplex) compared to a control (e.g., a cell, organ, tissue, system or subject that had not been contacted with or administered the compound or duplex, or was contacted with or administered a control substance (e.g., PBS)). In certain embodiments, the percentage of APOC3 RNA decrease or reduction in a cell (e.g., a hepatic cell), organ (e.g., a liver), tissue, system or subject (e.g., animal) contacted with or administered an oligomeric agent, oligomeric duplex or composition provided herein is 0.1% to 30% greater or less than, 0.1% to 25% greater or less than, 0.1% to 20% greater or less than, 0.1% to 15% greater or less than, 0.1% to 10% greater or less than, or 0.1% to 5% greater or less than, 0.1% to 1% greater or less than, 5% to 40% greater or less than, 5% to 35% greater or less than, 10% to 40% greater or less than, at least 5% greater than, at least 10% greater than, at least 15% greater than, at least 20% greater than, at least 25% greater than, or at least 30% greater than the percentage of APOC3 RNA decrease or reduction in a cell (e.g., a hepatic cell), organ (e.g., a liver), tissue, system or subject (e.g., animal) contacted with or administered the same concentration or dose of a comparator compound, e.g., having the same or similar nucleobase sequence and different modifications.

### VI. Pharmaceutical Compositions

In certain embodiments, described herein are pharmaceutical compositions comprising one or more oligomeric duplexes or oligomeric agents, wherein each oligomeric duplex or agent comprises a modified oligonucleotide (e.g., oligomeric compound). In certain embodiments, the one or more oligomeric duplex or oligomeric agent each comprises an antisense agent. In certain embodiments, a pharmaceutical composition comprises a pharmaceutically acceptable diluent or carrier. In certain embodiments, a pharmaceutical composition comprises or consists of a sterile saline solution and one or more compound or duplex. In certain embodiments, the sterile saline is pharmaceutical grade saline. In certain embodiments, a pharmaceutical composition comprises or consists of one or more compound or duplex and sterile water. In certain embodiments, the sterile water is pharmaceutical grade water. In certain embodiments, a pharmaceutical composition comprises or consists of one or more compound or duplex and phosphate-buffered saline (PBS). In certain embodiments, sterile PBS is pharmaceutical grade PBS.

In certain embodiments, a pharmaceutical composition comprises an oligomeric agent or oligomeric duplex comprising a first oligomeric compound and a second oligomeric compound; and sterile saline. In certain such embodiments, a pharmaceutical composition consists of such oligomeric agent or oligomeric duplex and sterile saline. In certain embodiments, a pharmaceutical composition consists essentially of such oligomeric agent or oligomeric duplex and sterile saline. In certain embodiments, the sterile saline is sterile PBS. In certain embodiments, the sterile saline is pharmaceutical grade.

In certain embodiments, pharmaceutical compositions comprise one or more oligomeric agent or oligomeric duplex and one or more excipients. In certain embodiments, excipients are selected from water, salt solutions, alcohol, polyethylene glycols, gelatin, lactose, amylase, magnesium stearate, talc, silicic acid, viscous paraffin, hydroxymethylcellulose and polyvinylpyrrolidone.

In certain embodiments, an oligomeric agent or oligomeric duplex may be admixed with pharmaceutically acceptable active and/or inert substances for the preparation of pharmaceutical compositions or formulations. Compositions and methods for the formulation of pharmaceutical compositions depend on a number of criteria, including, but not limited to, route of administration, extent of disease, or dose to be administered.

In certain embodiments, pharmaceutical compositions comprising an oligomeric agent or oligomeric duplex encompass any pharmaceutically acceptable salts of the compound or duplex, esters of the compound or duplex, or salts of such esters. In certain embodiments, pharmaceutical compositions comprising an oligomeric agent or oligomeric duplex comprising one or more oligomeric compound, upon administration to a subject, including a human, are capable of providing (directly or indirectly) the biologically active metabolite or residue thereof. Accordingly, for example, the disclosure is also drawn to pharmaceutically acceptable salts of oligomeric agents or oligomeric duplexes, and other bioequivalents. In certain embodiments, pharmaceutically acceptable salts comprise inorganic salts, such as monovalent or divalent inorganic salts. Suitable pharmaceutically acceptable salts include, but are not limited to, sodium, potassium, calcium, and magnesium salts.

In certain embodiments, oligomeric agents or oligomeric duplexes are lyophilized and isolated, e.g., as sodium salts. In certain embodiments, a sodium salt of an agent or duplex is mixed with a pharmaceutically acceptable diluent. In certain embodiments, the pharmaceutically acceptable diluent comprises sterile saline, sterile water, PBS. In certain embodiments, a sodium salt of an oligomeric agent or oligomeric duplex is mixed with PBS.

Lipid moieties have been used in nucleic acid therapies in a variety of methods. In certain methods, a nucleic acid, such as an oligomeric agent or oligomeric duplex comprising oligomeric compound, is introduced into preformed liposomes or lipoplexes made of mixtures of cationic lipids and neutral lipids. In certain methods, nucleic acid complexes with mono- or poly-cationic lipids are formed without the presence of a neutral lipid. In certain embodiments, a lipid moiety is selected to increase distribution of a pharmaceutical compound to a particular cell or tissue. In certain embodiments, a lipid moiety is selected to increase distribution of a pharmaceutical compound to fat tissue. In certain embodiments, a lipid moiety is selected to increase distribution of a pharmaceutical compound to muscle tissue.

In certain embodiments, pharmaceutical compositions comprise a delivery system. Examples of delivery systems include, but are not limited to, liposomes and emulsions. Certain delivery systems are useful for preparing certain pharmaceutical compositions including those comprising hydrophobic compounds. In certain embodiments, certain organic solvents such as dimethylsulfoxide are used.

In certain embodiments, pharmaceutical compositions comprise one or more tissue-specific delivery molecules designed to deliver the one or more compounds of the present invention to specific tissues or cell types. For example, in certain embodiments, pharmaceutical compositions include liposomes coated with a tissue-specific antibody.

In certain embodiments, pharmaceutical compositions comprise a co-solvent system. Certain of such co-solvent systems comprise, for example, benzyl alcohol, a nonpolar surfactant, a water-miscible organic polymer, and an aqueous phase. In certain embodiments, such co-solvent systems are used for hydrophobic compounds. A non-limiting example of such a co-solvent system is the VPD co-solvent system, which is a solution of absolute ethanol comprising 3% w/v benzyl alcohol, 8% w/v of the nonpolar surfactant Polysorbate 80™ and 65% w/v polyethylene glycol 300. The proportions of such co-solvent systems may be varied considerably without significantly altering their solubility and toxicity characteristics. Furthermore, the identity of co-solvent components may be varied: for example, other surfactants may be used instead of Polysorbate 80™; the fraction size of polyethylene glycol may be varied; other biocompatible polymers may replace polyethylene glycol, e.g., polyvinyl pyrrolidone; and other sugars or polysaccharides may substitute for dextrose.

In certain embodiments, pharmaceutical compositions are prepared for oral administration. In certain embodiments, pharmaceutical compositions are prepared for buccal administration. In certain embodiments, a pharmaceutical composition is prepared for administration by injection (e.g., intravenous, subcutaneous, intramuscular, intrathecal (IT), intracerebroventricular (ICV), etc.). In certain of such embodiments, a pharmaceutical composition comprises a carrier or diluent and is formulated in aqueous solution, such as water or physiologically compatible buffers such as Hanks's solution, Ringer's solution, or physiological saline buffer. In certain embodiments, other ingredients are included (e.g., ingredients that aid in solubility or serve as preservatives). In certain embodiments, injectable suspensions are prepared using appropriate liquid carriers, diluents, suspending agents and the like. Certain pharmaceutical compositions for injection are presented in unit dosage form, e.g., in ampoules or in multi-dose containers. Certain pharmaceutical compositions for injection are suspensions, solutions or emulsions in oily or aqueous vehicles, and may contain formulatory agents such as suspending, stabilizing and/or dispersing agents. Certain solvents suitable for use in pharmaceutical compositions for injection include, but are not limited to, lipophilic solvents and fatty oils, such as sesame oil, synthetic fatty acid esters, such as ethyl oleate or triglycerides, and liposomes.

Under certain conditions, certain compounds disclosed herein act as acids. Although such compounds may be drawn or described in protonated (free acid) form or ionized and in association with a cation (salt) form, aqueous solutions of such compounds exist in equilibrium among such forms. For example, a phosphodiester linkage of an oligonucleotide in aqueous solution exists in equilibrium among free acid, anion and salt forms. Unless otherwise indicated, compounds described herein are intended to include all such forms. Moreover, certain oligonucleotides have several such linkages, each of which is in equilibrium. Thus, oligonucleotides in solution exist in an ensemble of forms at multiple positions all at equilibrium. The term “oligonucleotide” herein is intended to include all such forms. Drawn structures necessarily depict a single form. Nevertheless, unless otherwise indicated, such drawings are likewise intended to include corresponding forms. Herein, a structure depicting the free acid of a compound followed by the term “or a pharmaceutically acceptable salt thereof” expressly includes all such forms that may be fully or partially protonated/de-protonated/in association with a cation or a combination of cations. In certain embodiments, one or more specific cation is identified. The cations include, but are not limited to, sodium, potassium, calcium, and magnesium. In certain embodiments, a structure depicting the free acid of a compound followed by the term “or a pharmaceutically acceptable salt thereof” expressly includes all such forms that may be fully or partially protonated/de-protonated/in association with one or more cations selected from sodium, potassium, calcium, and magnesium.

In certain embodiments, oligomeric agents, modified oligonucleotides (e.g., oligomeric compounds), or oligomeric duplexes are in aqueous solution with sodium. In certain embodiments, oligomeric agents, modified oligonucleotides, oligomeric compounds or oligomeric duplexes are in aqueous solution with potassium. In certain embodiments, oligomeric agents, oligomeric compounds, modified oligonucleotides or oligomeric duplexes are in PBS. In certain embodiments, oligomeric agents, modified oligonucleotides, oligomeric compounds, or oligomeric duplexes are in water. In certain such embodiments, the pH of a solution is adjusted with NaOH and/or HCl to achieve a desired pH.

Herein, a dose may be in the form of a dosage unit. For clarity, a dose (or dosage unit) of an agent (e.g., modified oligonucleotide, oligomeric duplex, antisense agent) in milligrams indicates the mass of the free acid form of the compound. As described herein, in aqueous solution, the free acid is in equilibrium with anionic and salt forms. However, for the purpose of calculating dose, it is assumed that the compound (e.g., modified oligonucleotide, oligomeric duplex, antisense agent) exists as a solvent-free, sodium-acetate free, anhydrous, free acid. In certain embodiments, where an agent (e.g., modified oligonucleotide, oligomeric duplex, antisense agent) is in solution comprising sodium (e.g., saline), the compound may be partially or fully de-protonated and in association with sodium ions. However, the mass of the protons is nevertheless counted toward the weight of the dose, and the mass of the sodium ions is not counted toward the weight of the dose. When an agent comprises a conjugate group, the mass of the conjugate group is included in calculating the dose of such compound. If the conjugate group also has an acid, the conjugate group is likewise assumed to be fully protonated for the purpose of calculating dose.

### VII. Compounds

Provided herein are reduced fluorine content agents and duplexes. In certain embodiments, a reduced fluorine content oligomeric compound comprises an oligonucleotide (e.g., an antisense oligomeric compound) which has a nucleobase sequence complementary to a sequence in a APOC3 nucleic acid, e.g., a human APOC3 nucleic acid (SEQ ID NO: 1), or an oligonucleotide (e.g., a sense oligomeric compound) which has a nucleobase sequence complementary to a sequence of an oligonucleotide which has a nucleobase sequence complementary to a sequence in a APOC3 nucleic acid, e.g., a human APOC3 nucleic acid (SEQ ID NO: 1). In certain embodiments, a reduced fluorine content oligomeric duplex comprises a first modified oligonucleotide (e.g., an antisense oligomeric compound), which has a nucleobase sequence complementary to a sequence of a APOC3 nucleic acid (e.g., human APOC3 nucleic acid (SEQ ID NO: 1)), and a second modified oligonucleotide (e.g., a sense oligomeric compound), which has a nucleobase sequence complementary to a sequence of the first modified oligonucleotide. In certain embodiments, the reduced fluorine content oligomeric compounds and oligomeric duplexes provided herein may be preferable to compounds containing more fluorine atoms due to improved properties, e.g., decreased off-target actions and/or improved durability, and have APOC3 RNA and/or ApoCIII protein reduction activity that is comparable to or greater than that of a comparator compound containing more fluorine atoms (e.g., a compound having 20% or more, 25% or more, or 30% or more fluorine-containing nucleosides). In certain embodiments, an oligomeric compound or oligomeric duplex having reduced fluorine content provided herein comprises a modified oligonucleotide or a first modified oligonucleotide which has a nucleobase sequence complementary to a sequence in a APOC3 nucleic acid, having reduced fluorine content has fewer than 20%, fewer than 15%, fewer than 10%, or fewer than 5% of nucleosides comprising a fluorine atom, and/or a modified oligonucleotide or a second modified oligonucleotide which has a nucleobase sequence complementary to the first oligonucleotide, or to a sequence that is complementary to a sequence in a APOC3 nucleic acid, having reduced fluorine content has fewer than 10%, fewer than 8%, or fewer than 5% of nucleosides comprising a fluorine atom. In certain embodiments, an oligomeric compound or oligomeric duplex having reduced fluorine content provided herein comprises a modified oligonucleotide or first modified oligonucleotide having very low fluorine content has fewer than 20%, fewer than 15%, fewer than 10% or fewer than 5% of nucleosides comprising a fluorine atom. In certain embodiments, the second modified oligonucleotide of such oligomeric duplexes has a very low fluorine content has fewer than 15%, fewer than 12%, or fewer than 10% of nucleosides comprising a fluorine atom. In certain embodiments, an oligomeric compound or oligomeric duplex provided herein comprises an oligonucleotide or first modified oligonucleotide (e.g., an antisense oligomeric compound) having reduced fluorine content has fewer than 20%, fewer than 15%, fewer than 10%, or fewer than 5% of nucleosides comprising a fluorine atom, and has a nucleobase sequence that is at least 85%, at least 90%, at least 95%, or at least 99% complementary to an equal length portion of a APOC3 nucleic acid selected from a nucleobase sequence of any one of SEQ ID NOs: 2-5. In certain embodiments, an oligomeric compound or oligomeric duplex provided herein comprises an oligonucleotide or a first modified oligonucleotide (e.g., an antisense oligomeric compound), having reduced fluorine content has fewer than 20%, fewer than 15%, fewer than 10%, or fewer than 5% of nucleosides comprising a fluorine atom, and having a nucleobase sequence selected from among nucleobase sequence of any one of SEQ ID NOs: 2-5. In certain embodiments, such oligomeric compounds or oligomeric duplexes comprise an oligonucleotide or second modified oligonucleotide (e.g., a sense oligomeric compound) having reduced fluorine content, wherein fewer than 20%, fewer than 15%, or fewer than 10% of nucleosides comprising a fluorine atom, comprising a nucleobase sequence complementary to the first modified oligonucleotide selected from among a nucleobase sequence of any one of SEQ ID NOs: 2-5. In some such embodiments, the modified oligonucleotide or first modified oligonucleotide has reduced fluorine content, wherein less than 20%, less than 15%, less than 10% or less than 5% of nucleobases comprising a fluorine atom, and comprises at least 18, at least 19, at least 20, at least 21, at least 22, or at least 23 contiguous nucleobases of the nucleobase sequence of any one of SEQ ID NOs: 2-5; and the modified oligonucleotide or second modified oligonucleotide has reduced fluorine content, wherein less than 20% less than 15%, or less than 10% of nucleobases comprising a fluorine atom, and comprises at least 16, at least 17, at least 18, at least 19, at least 20, or at least 21 contiguous nucleobases of the nucleobase sequence of any one of SEQ ID NOs: 6-8. In certain embodiments, an oligomeric compound or oligomeric duplex having reduced fluorine content provided herein comprises a modified oligonucleotide or first modified oligonucleotide having very low fluorine content, wherein fewer than 12%, fewer than 10% or fewer than 5% of nucleosides comprising a fluorine atom, and comprises at least 18, at least 19, at least 20, at least 21, at least 22, or at least 23 contiguous nucleobases of the nucleobase sequence of any one of SEQ ID NOs: 2-5. In certain embodiments, the second modified oligonucleotide of such oligomeric duplexes has a very low fluorine content, wherein fewer than 15%, fewer than 12%, or fewer than 10% of nucleosides comprising a fluorine atom, and comprises at least 16, at least 17, at least 18, at least 19, at least 20, or at least 21 contiguous nucleobases of the nucleobase sequence of any one of SEQ ID NOs: 6-8.

In certain embodiments, an oligomeric compound or oligomeric duplex having reduced fluorine content provided herein comprises a conjugate group. In some such embodiments of oligomeric duplexes provided herein, the conjugate group is attached to the first (e.g., antisense) or second (e.g., sense) modified oligonucleotide of the oligomeric duplex. In certain embodiments, the conjugate group is attached to the 5′- or 3′-end of the modified oligonucleotide of an oligomeric compound or of the first or second modified oligonucleotide of an oligomeric duplex, or the 5′- or 3′-terminal nucleoside of the modified oligonucleotide of an oligomeric compound or of the first or second modified oligonucleotide of an oligomeric duplex. In certain embodiments, the conjugate group is attached to the second modified oligonucleotide (e.g., sense oligomeric compound), for example, the 5′- or 3′-terminal nucleoside of the second modified oligonucleotide of an oligomeric duplex. In certain embodiments, the conjugate group is attached to the 5′-terminal nucleoside of the second modified oligonucleotide. In certain embodiments, the conjugate group comprises a cell-targeting moiety having affinity for the hepatic asialoglycoprotein receptor (ASGP-R). In certain embodiments, the cell-targeting moiety comprises more than one ligand, each an N-acetyl galactosamine (GalNAc). In certain embodiments, the cell-targeting moiety comprises 3 GalNAc ligands.

In certain embodiments, the conjugate group has the following structure:

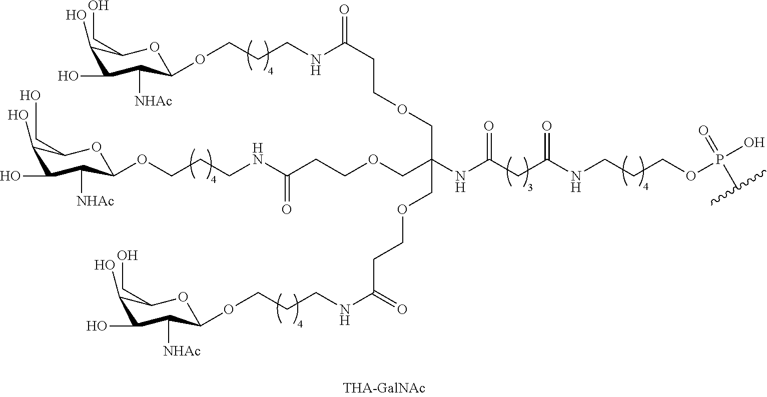

and is attached to the second modified oligonucleotide (e.g., sense oligomeric compound) of the oligomeric duplex through a phosphodiester bond, e.g., through a phosphodiester bond with the 5′-terminal nucleoside of the modified oligonucleotide of the oligomeric compound or the second modified oligonucleotide of the oligomeric duplex. In certain embodiments, the conjugate group has the following structure:

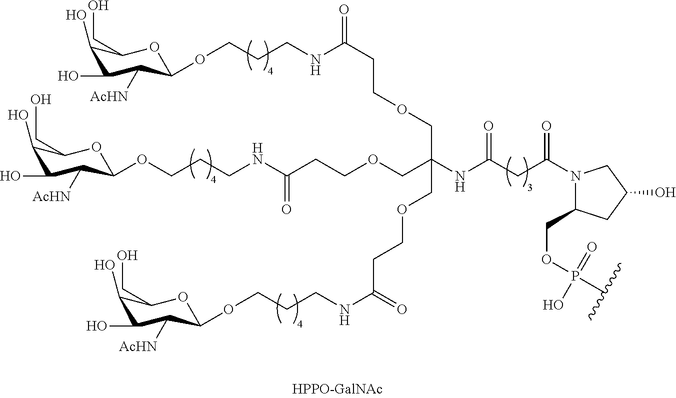

and is attached to the second modified oligonucleotide (e.g., sense oligomeric compound) of the oligomeric duplex through a phosphodiester bond, e.g., through a phosphodiester bond with the 3′-terminal nucleoside of the modified oligonucleotide of the oligomeric compound or the second modified oligonucleotide of the oligomeric duplex.
a. Compound No. 1692958

Provided herein is Compound No. 1692958, which is an oligomeric duplex that consists of a first oligomeric compound containing a first modified oligonucleotide and a second oligomeric compound containing a second modified oligonucleotide attached to a conjugate group as follows.

### First Oligomeric Compound of Compound No. 1692958:

The first oligomeric compound of Compound no. 1692958, which is Compound No. 1692954, has a first modified oligonucleotide having a nucleobase sequence of (from 5′ to 3′) TCACUGAGAAUACUGUCCCUUUU (SEQ ID NO: 2), wherein nucleoside 1 (from 5′ to 3′) comprises a 2′-MOE modified sugar moiety, each of nucleosides 2, 6, 14 and 16 (from 5′ to 3′) comprises a 2′-fluoro modified sugar moiety, each of nucleosides 3-5, 7-13, 15 and 17-23 (from 5′ to 3′) comprises a 2′-OMe modified sugar moiety, wherein each of the internucleoside linkages linking nucleosides 1 to 2, 2 to 3, 21 to 22, and 22 to 23 (from 5′ to 3′) is a phosphorothioate internucleoside linkage and each of the internucleoside linkages linking nucleosides 3 to 4, 4 to 5, 5 to 6, 6 to 7, 7 to 8, 8 to 9, 9 to 10, 10 to 11, 11 to 12, 12 to 13, 13 to 14, 14 to 15, 15 to 16, 16 to 17, 17 to 18, 18 to 19, 19 to 20, and 20 to 21 (from 5′ to 3′) is a phosphodiester linkage, and wherein nucleoside 1 (from 5′ to 3′) is attached to a vinyl phosphonate moiety.

The first oligomeric compound of Compound 1692958 is represented by the following chemical notation: VP-TₑₛC_{fs}A_{yo}C_{yo}U_{yo}G_{fo}A_{yo}G_{yo}A_{yo}A_{yo}U_{yo}A_{yo}C_{yo}U_{fo}G_{yo}U_{fo}C_{yo}C_{yo}C_{yo}U_{yo}U_{ys}U_{ys}U_{y} (SEQ ID NO: 11); wherein:
A=an adenine nucleobase,
C=a cytosine nucleobase,
G=a guanine nucleobase,
T=a thymine nucleobase,
U=a uracil nucleobase,
e=a 2′-MOE sugar moiety,
f=a 2′-fluoro sugar moiety,
y=a 2′-OMe sugar moiety,
o=a phosphodiester internucleoside linkage,
s=a phosphorothioate internucleoside linkage, and
VP=a 5′ vinyl phosphonate moiety.

### Second Oligomeric Compound of Compound No. 1692958:

The second oligomeric compound of Compound No. 1692958, which is Compound No. 1692955, has a nucleobase sequence of (from 5′ to 3′) AAGGGACAGUAUUCUCAGUGA (SEQ ID NO: 6), wherein each of nucleosides 7, 9, 10 and 11 (from 5′ to 3′) comprises a 2′-fluoro modified sugar moiety, and each of nucleosides 1-6, 8, and 12-21 (from 5′ to 3′) comprises a 2′-OMe modified sugar moiety, wherein each of the internucleoside linkages linking nucleosides 1 to 2, 2 to 3, 19 to 20, and 20 to 21 (from 5′ to 3′) is a phosphorothioate internucleoside linkage and each of the internucleoside linkages linking nucleosides 3 to 4, 4 to 5, 5 to 6, 6 to 7, 7 to 8, 8 to 9, 9 to 10, 10 to 11, 11 to 12, 12 to 13, 13 to 14, 14 to 15, 15 to 16, 16 to 17, 17 to 18, and 18 to 19 (from 5′ to 3′) is a phosphodiester linkage, and wherein nucleoside 1 (from 5′ to 3′) is attached is attached through the 5′-OH of the oligonucleotide to a conjugate group comprising [THA-GalNAc].

The second oligomeric compound of Compound 1692958 is represented by the following chemical notation: [THA-GalNAc]-A_{ys}A_{ys}G_{yo}G_{yo}G_{yo}A_{yo}C_{fo}A_{yo}G_{fo}U_{fo}A_{fo}U_{yo}U_{yo}C_{yo}U_{yo}C_{yo}A_{yo}G_{yo}U_{ys}G_{ys}A_{y} (SEQ ID NO: 35); wherein:

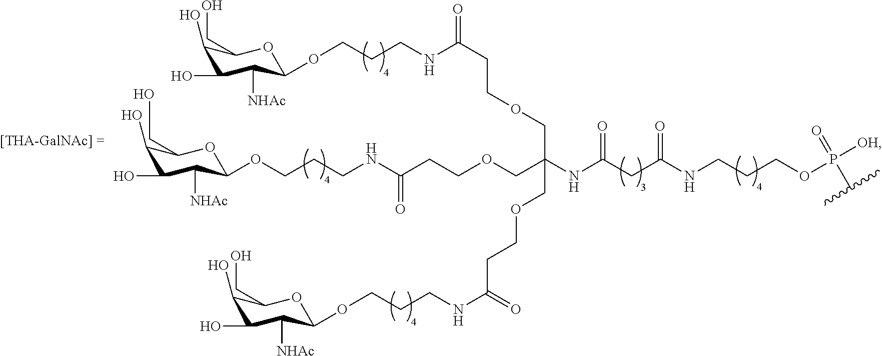

A=an adenine nucleobase,
C=a cytosine nucleobase,
G=a guanine nucleobase,
T=a thymine nucleobase,
U=a uracil nucleobase,
f=a 2′-fluoro sugar moiety,
y=a 2′-OMe sugar moiety,
o=a phosphodiester internucleoside linkage, and
s=a phosphorothioate internucleoside linkage.

The following chemical structure is one structural representation of Compound No. 1692958:

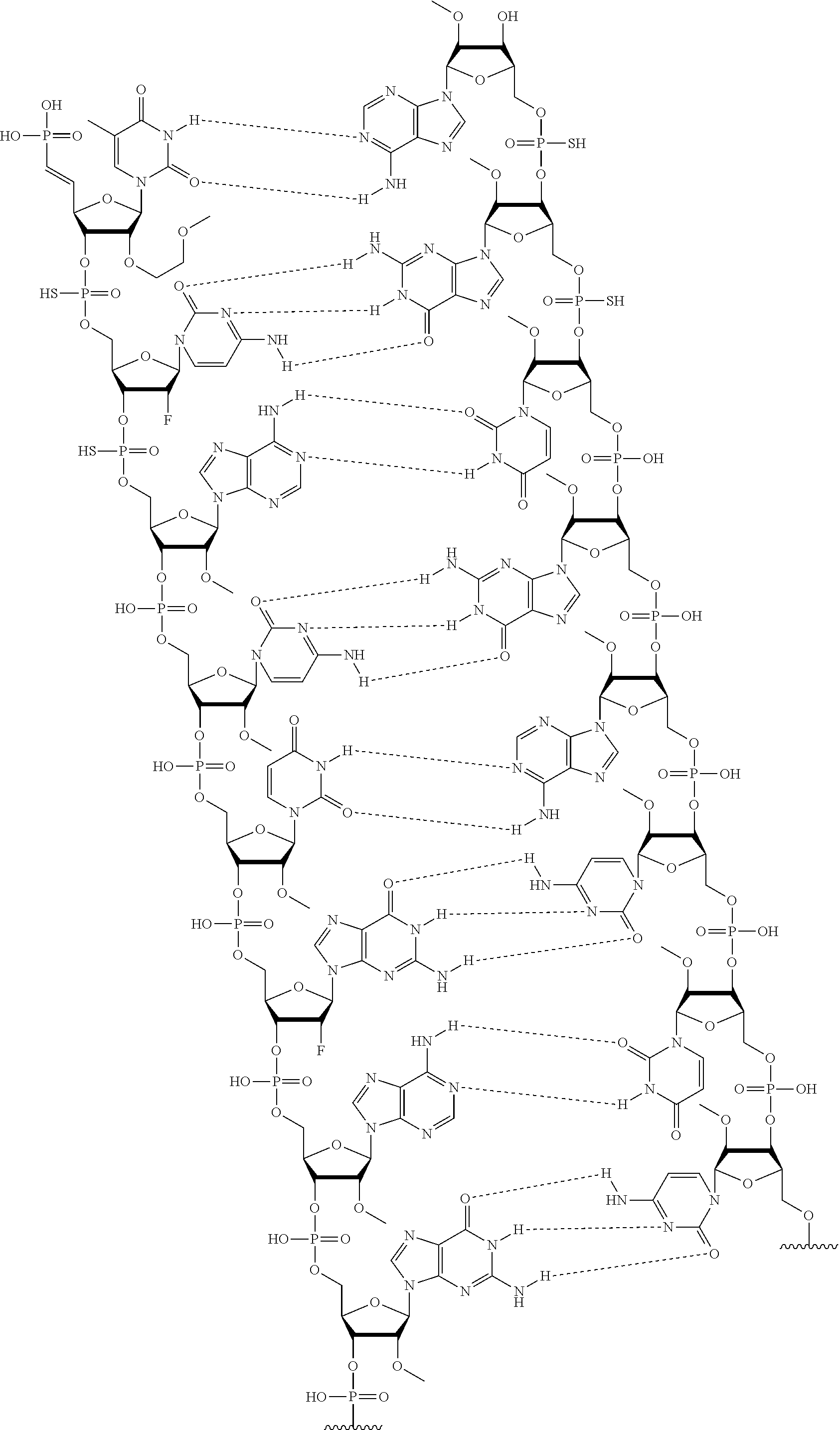

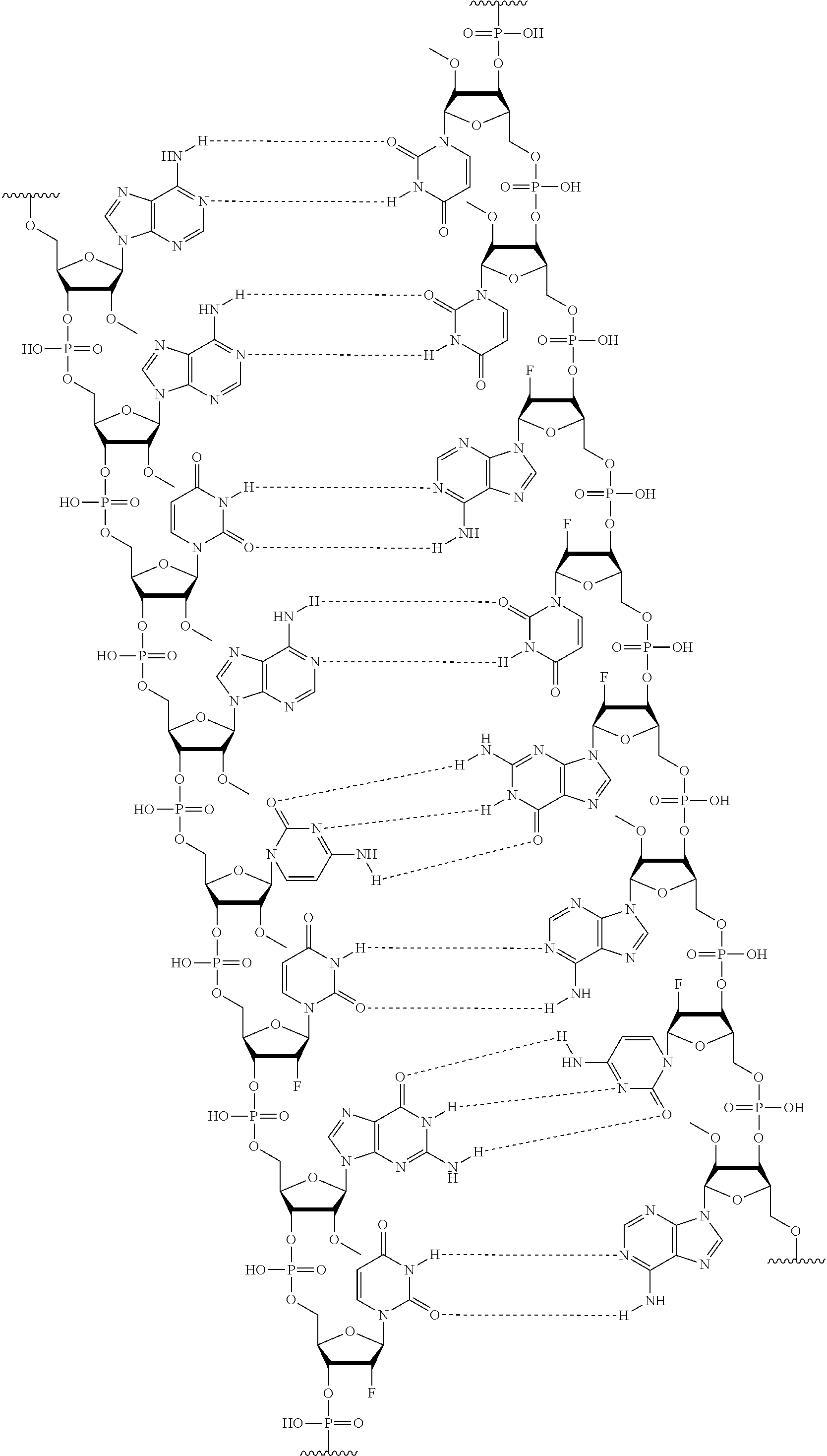

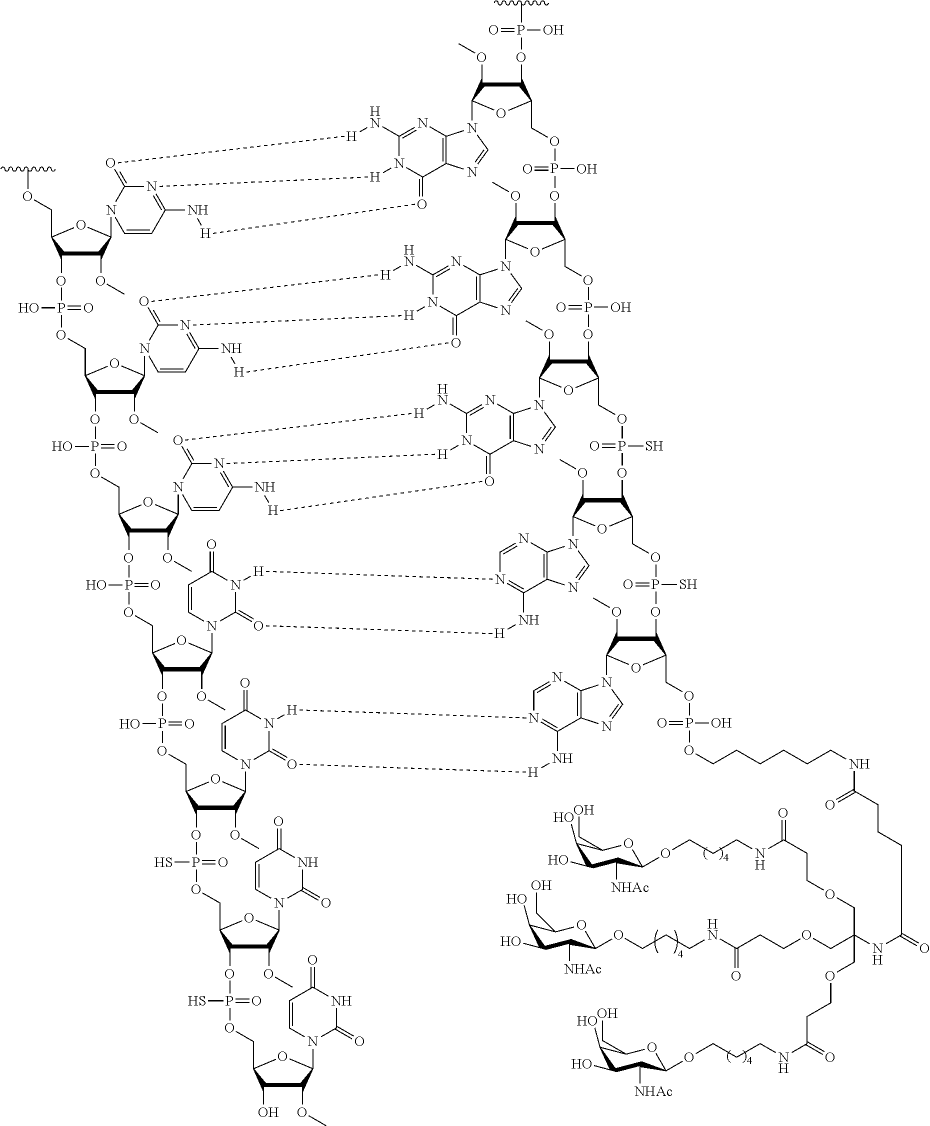

(SEQ ID NO: 11 and SEQ ID NO: 35), or an ion or salt thereof.

In certain embodiments, Compound No. 1692958 is in the form of an anion or a salt thereof, for example, a sodium salt. In certain embodiments, the oligomeric duplex is in anionic form in a solution. In certain embodiments, Compound No. 1692958 is a sodium salt or a potassium salt.

The following chemical structure represents Compound No. 1692958 in sodium solution:

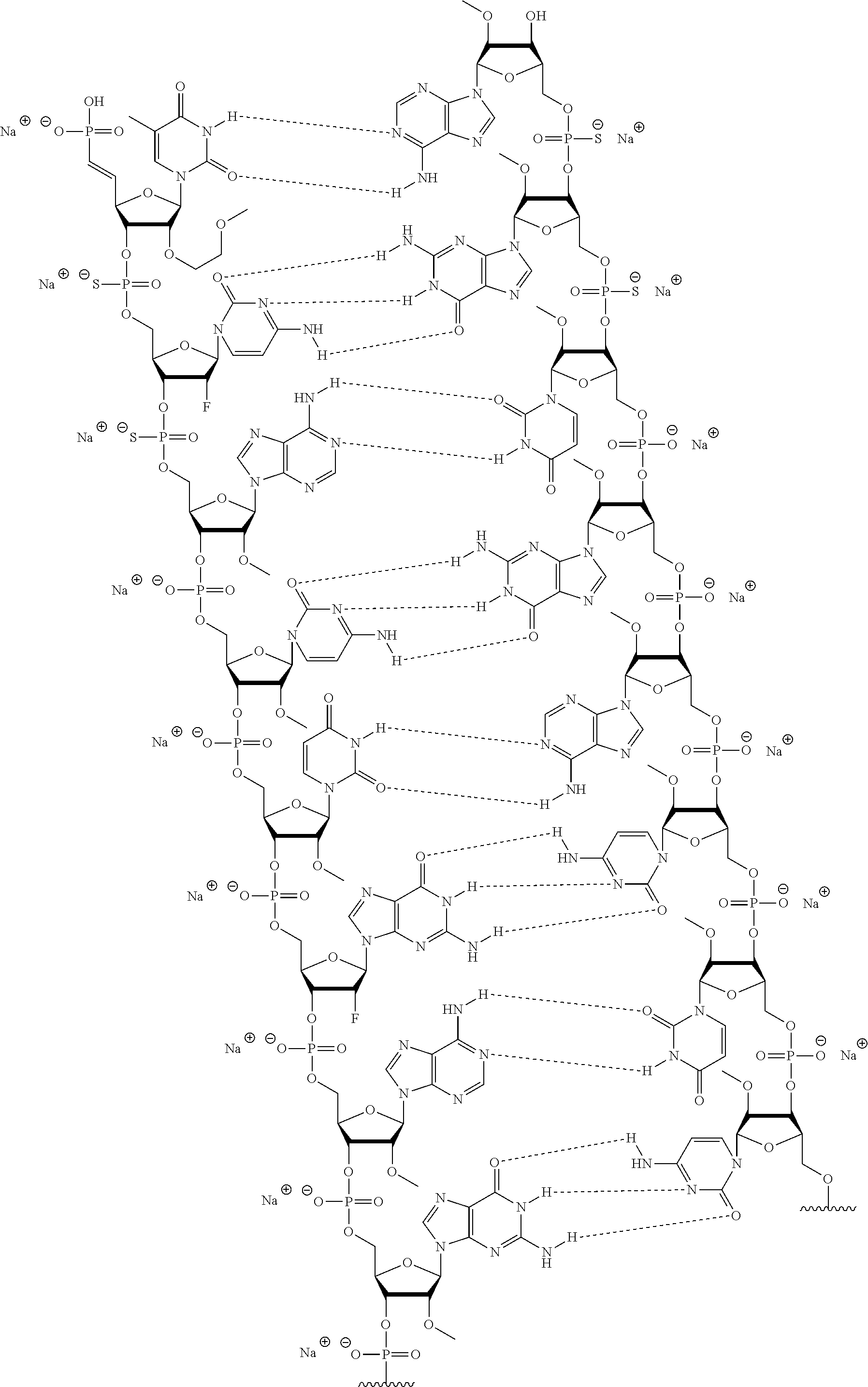

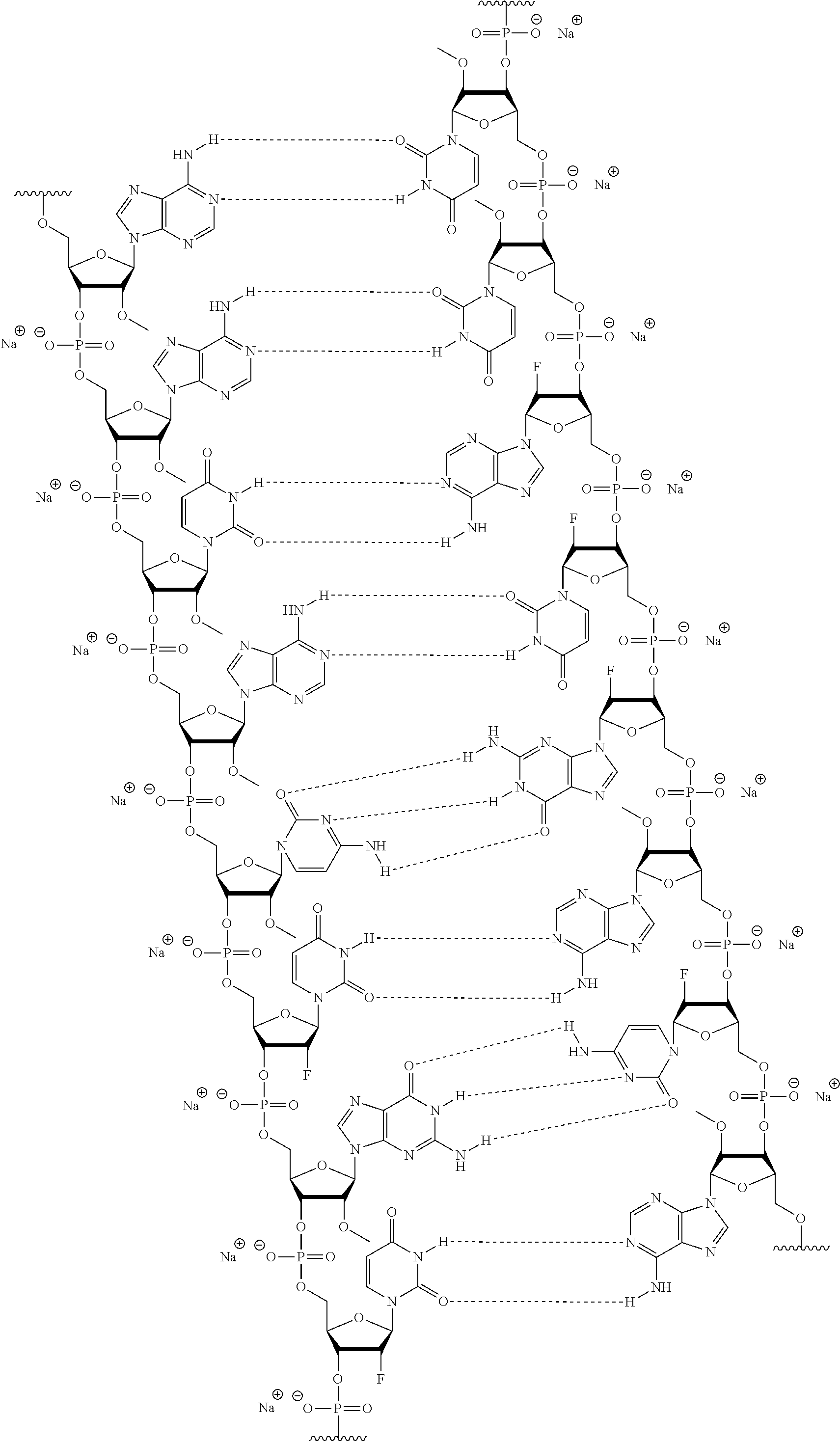

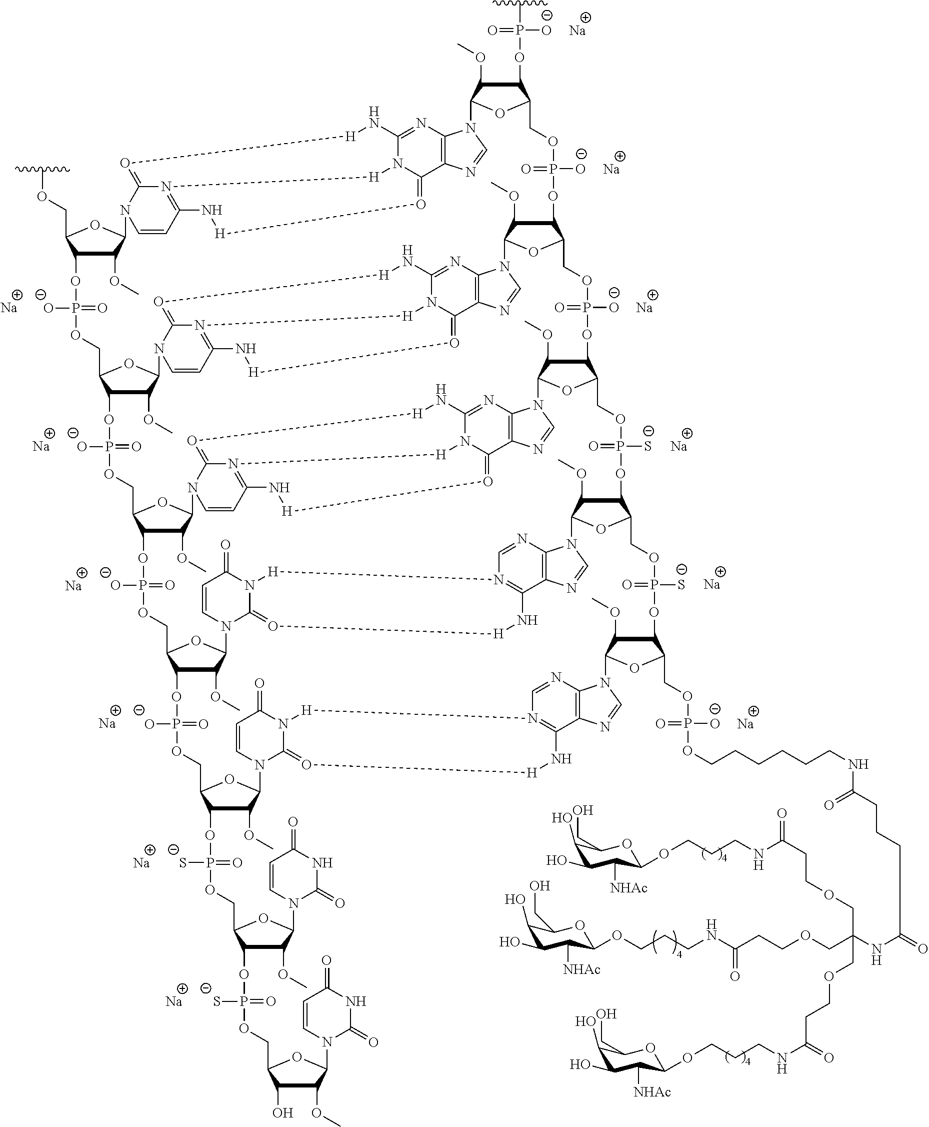

### (SEQ ID NO: 11 and SEQ ID NO: 35).

In certain embodiments, provided herein are oligomeric duplexes comprising or consisting of a first oligomeric compound containing a first modified oligonucleotide and a second oligomeric compound containing a second modified oligonucleotide as follows.

The first oligomeric compound is Compound No. 1692954, as described in a. above for Compound no. 1692958.

The second oligomeric compound has a nucleobase sequence of (from 5′ to 3′) AAGGGACAGUAUUCUCAGUGA (SEQ ID NO: 6), wherein each of nucleosides 7, 9, 10 and 11 (from 5′ to 3′) comprises a 2′-fluoro modified sugar moiety, and each of nucleosides 1-6, 8, and 12-21 (from 5′ to 3′) comprises a 2′-OMe modified sugar moiety, wherein each of the internucleoside linkages linking nucleosides 1 to 2, 2 to 3, 19 to 20, and 20 to 21 (from 5′ to 3′) is a phosphorothioate internucleoside linkage and each of the internucleoside linkages linking nucleosides 3 to 4, 4 to 5, 5 to 6, 6 to 7, 7 to 8, 8 to 9, 9 to 10, 10 to 11, 11 to 12, 12 to 13, 13 to 14, 14 to 15, 15 to 16, 16 to 17, 17 to 18, and 18 to 19 (from 5′ to 3′) is a phosphodiester linkage.

The second oligomeric compound is represented by the following chemical notation: A_{ys}A_{ys}G_{yo}G_{yo}G_{yo}A_{yo}C_{fo}A_{yo}G_{fo}U_{fo}A_{fo}U_{yo}U_{yo}C_{yo}U_{yo}C_{yo}A_{yo}G_{yo}U_{ys}G_{ys}A_{y} (SEQ ID NO: 75); wherein:
A=an adenine nucleobase,
C=a cytosine nucleobase,
G=a guanine nucleobase,
T=a thymine nucleobase,
U=a uracil nucleobase,
f=a 2′-fluoro sugar moiety,
y=a 2′-OMe sugar moiety,
o=a phosphodiester internucleoside linkage, and
s=a phosphorothioate internucleoside linkage.

In certain embodiments, such an oligomeric duplex is represented by the following chemical structure:

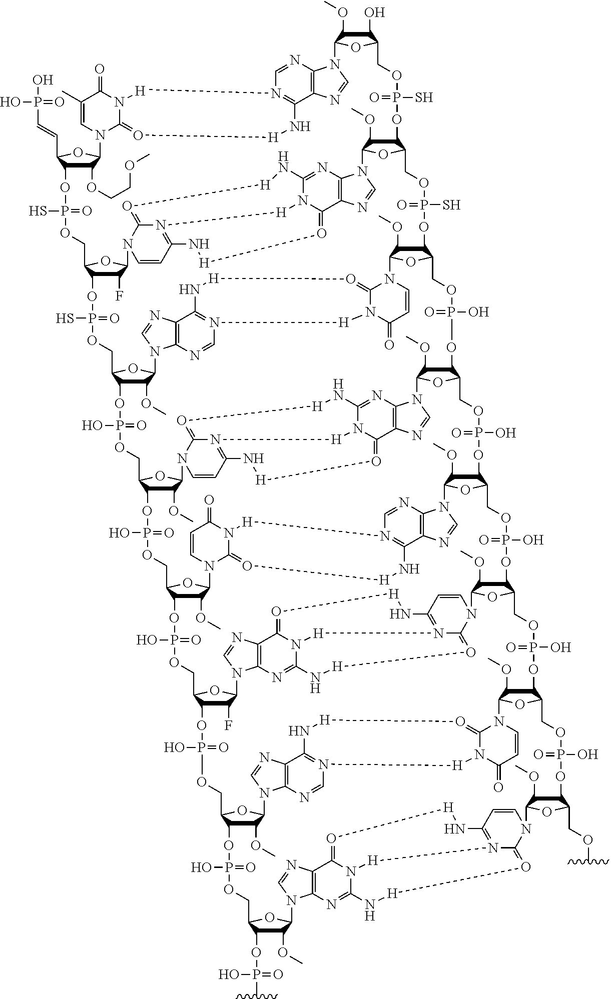

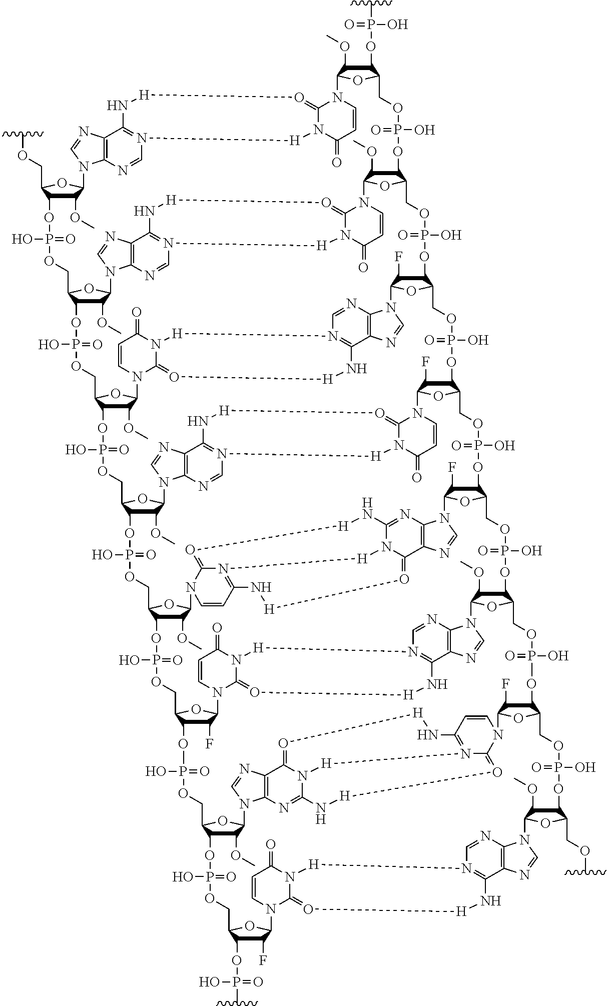

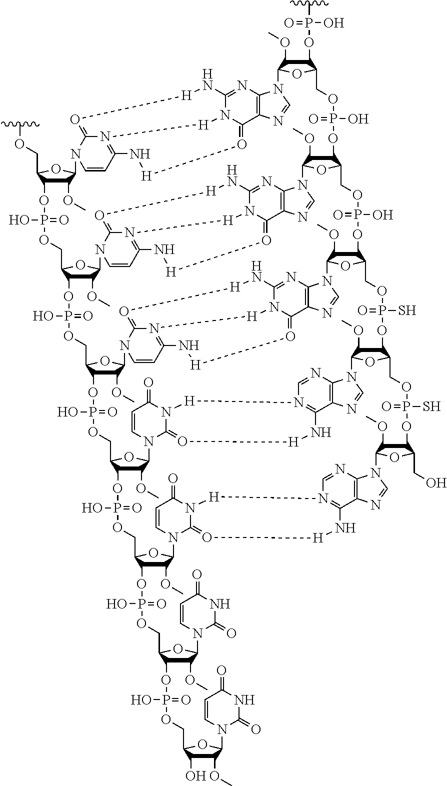

(SEQ ID NO: 11 and SEQ ID NO: 75), or an ion or salt thereof.

In certain embodiments, such an oligomeric duplex is in the form of an anion or a salt thereof, for example, a sodium salt. In certain embodiments, the oligomeric duplex is in anionic form in a solution. In certain embodiments, the oligomeric duplex is a sodium salt or a potassium salt.

The following chemical structure represents such an oligomeric duplex in sodium solution:

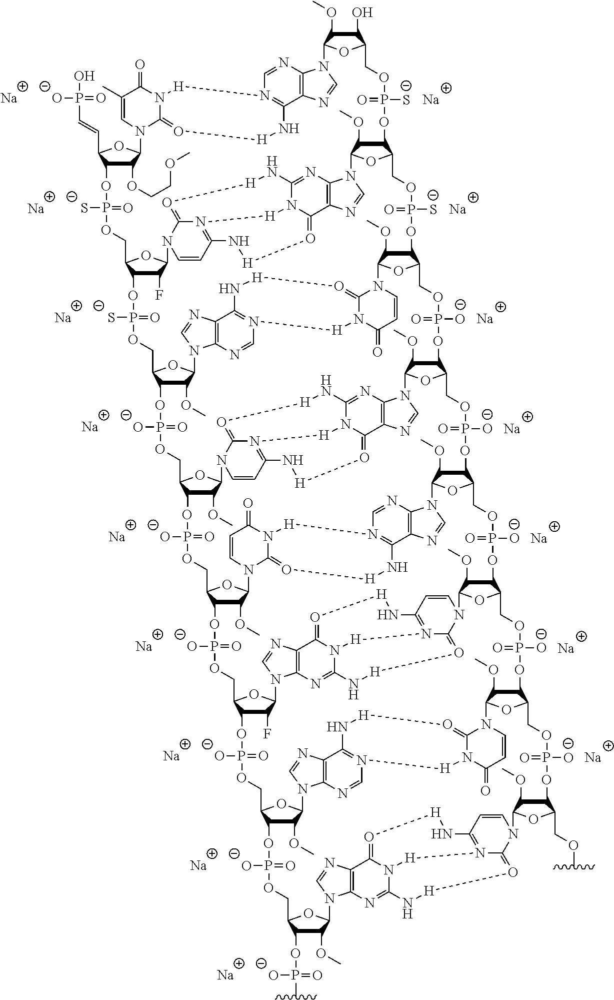

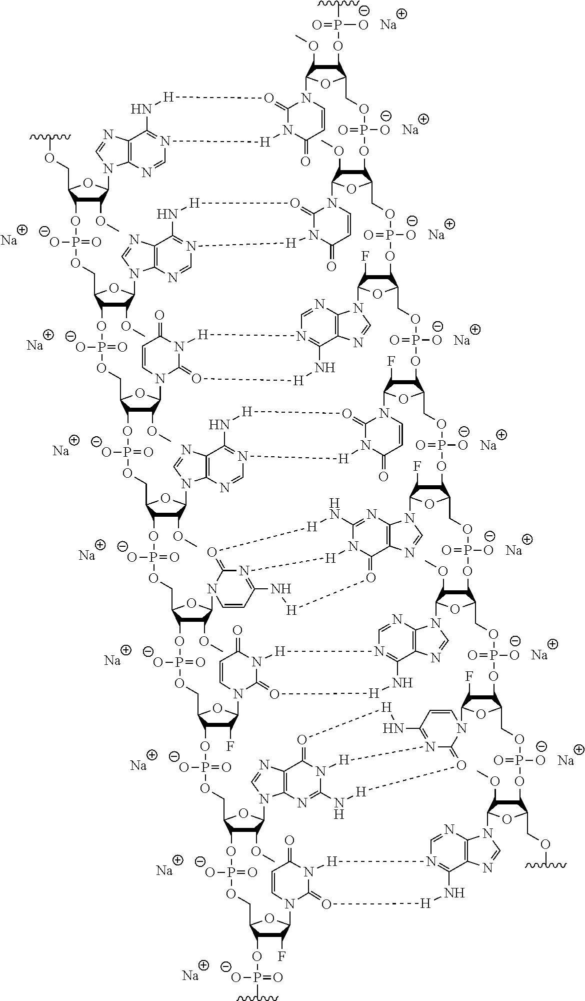

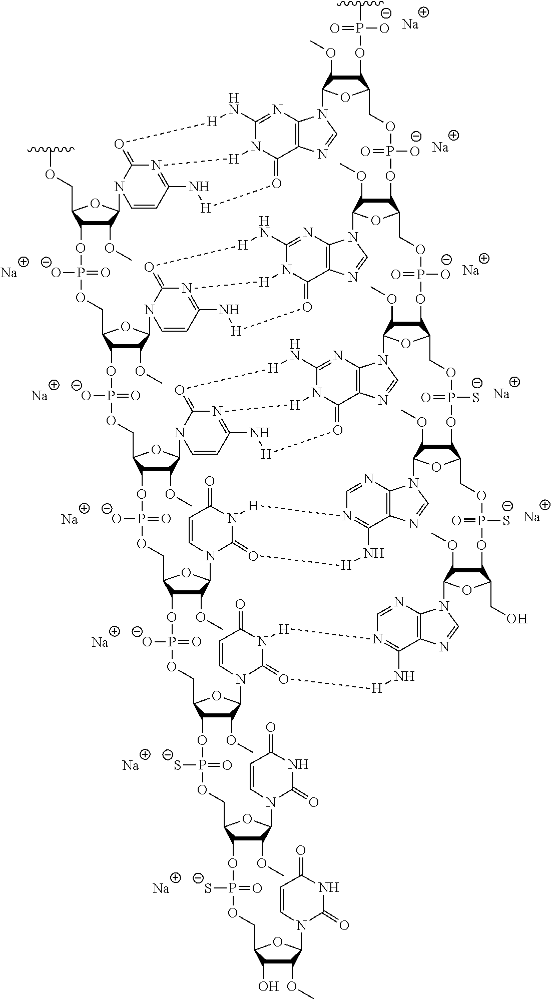

### (SEQ ID NO: 11 and SEQ ID NO: 75).

b. Compound No. 1754976

Provided herein is Compound No. 1754976, which is an oligomeric duplex that consists of a first oligomeric compound containing a first modified oligonucleotide and a second oligomeric compound containing a second modified oligonucleotide attached to a conjugate group as follows.

### First Oligomeric Compound of Compound No. 1754976:

The first oligomeric compound of Compound No. 1754976, which is Compound No. 1753166, has a first modified oligonucleotide having a nucleobase sequence of (from 5′ to 3′) TCACUGAGAAUACTGTCCCUUAA (SEQ ID NO: 4), wherein each of nucleosides 1, 9, 22 and 23 (from 5′ to 3′) comprises a 2′-MOE modified sugar moiety, nucleoside 2 (from 5′ to 3′) comprises a 2′-fluoro modified sugar moiety, each of nucleosides 3-5, 7, 8, 10-13, 15 and 17-21 (from 5′ to 3′) comprises a 2′-OMe modified sugar moiety, and each of nucleosides 6, 14 and 16 (from 5′ to 3′) is a 2′-deoxynucleoside, wherein each of the internucleoside linkages linking nucleosides 1 to 2, 2 to 3, 21 to 22, and 22 to 23 (from 5′ to 3′) is a phosphorothioate internucleoside linkage and each of the internucleoside linkages linking nucleosides 3 to 4, 4 to 5, 5 to 6, 6 to 7, 7 to 8, 8 to 9, 9 to 10, 10 to 11, 11 to 12, 12 to 13, 13 to 14, 14 to 15, 15 to 16, 16 to 17, 17 to 18, 18 to 19, 19 to 20, and 20 to 21 (from 5′ to 3′) is a phosphodiester linkage, and wherein nucleoside 1 (from 5′ to 3′) is attached to a vinyl phosphonate moiety.

The first oligomeric compound of Compound 1754976 is represented by the following chemical notation: VP-TₑₛC_{fs}A_{yo}C_{yo}U_{yo}G_{do}A_{yo}G_{yo}AₑₒA_{yo}U_{yo}A_{yo}C_{yo}T_{do}G_{yo}T_{do}C_{yo}C_{yo}C_{yo}U_{yo}U_{ys}AₑₛAₑ (SEQ ID NO: 28); wherein
A=an adenine nucleobase,
C=a cytosine nucleobase,
G=a guanine nucleobase,
T=a thymine nucleobase,
U=a uracil nucleobase,
d=a 2′-β-D-deoxyribosyl sugar moiety,
e=a 2′-MOE sugar moiety,
f=a 2′-fluoro sugar moiety,
y=a 2′-OMe sugar moiety,
o=a phosphodiester internucleoside linkage,
s=a phosphorothioate internucleoside linkage, and
VP=a 5′ vinyl phosphonate moiety.

### Second Oligomeric Compound of Compound No. 1754976:

The second oligomeric compound of Compound No. 1754976, which is Compound No. 1735443, has a nucleobase sequence of (from 5′ to 3′) AAGGGACAGUAUUCUCAGUGA (SEQ ID NO: 6), wherein each of nucleosides 1, 2, 20 and 21 (from 5′ to 3′) comprises a 2′-MOE modified sugar moiety, each of nucleosides 10 and 11 (from 5′ to 3′) comprises a 2′-fluoro modified sugar moiety, and each of nucleosides 3-9, and 12-19 (from 5′ to 3′) comprises a 2′-OMe modified sugar moiety, wherein each of the internucleoside linkages linking nucleosides 1 to 2, 2 to 3, 10 to 11, 19 to 20, and 20 to 21 (from 5′ to 3′) is a phosphorothioate internucleoside linkage and each of the internucleoside linkages linking nucleosides 3 to 4, 4 to 5, 5 to 6, 6 to 7, 7 to 8, 8 to 9, 9 to 10, 11 to 12, 12 to 13, 13 to 14, 14 to 15, 15 to 16, 16 to 17, 17 to 18, and 18 to 19 (from 5′ to 3′) is a phosphodiester linkage, and wherein nucleoside 1 (from 5′ to 3′) is attached is attached through the 5′-OH of the oligonucleotide to a conjugate group comprising [THA-GalNAc].

The second oligomeric compound of Compound 1754976 is represented by the following chemical notation: [THA-GalNAc]-AₑₛAₑₛG_{yo}G_{yo}G_{yo}A_{yo}C_{yo}A_{yo}G_{yo}U_{fs}A_{fo}U_{yo}U_{yo}C_{yo}U_{yo}C_{yo}A_{yo}G_{yo}U_{ys}GₑₛAₑ (SEQ ID NO: 40); wherein:

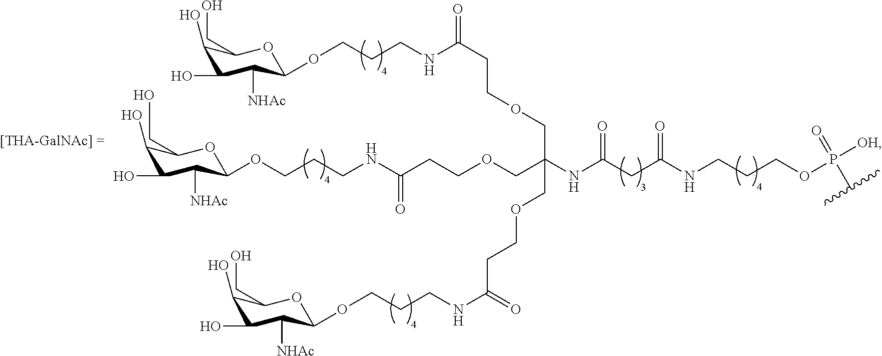

A=an adenine nucleobase,
C=a cytosine nucleobase,
G=a guanine nucleobase,
T=a thymine nucleobase,
U=a uracil nucleobase,
e=a 2′-MOE sugar moiety,
f=a 2′-fluoro sugar moiety,
y=a 2′-OMe sugar moiety,
o=a phosphodiester internucleoside linkage, and
s=a phosphorothioate internucleoside linkage.

The following chemical structure is one structural representation of Compound No. 1754976:

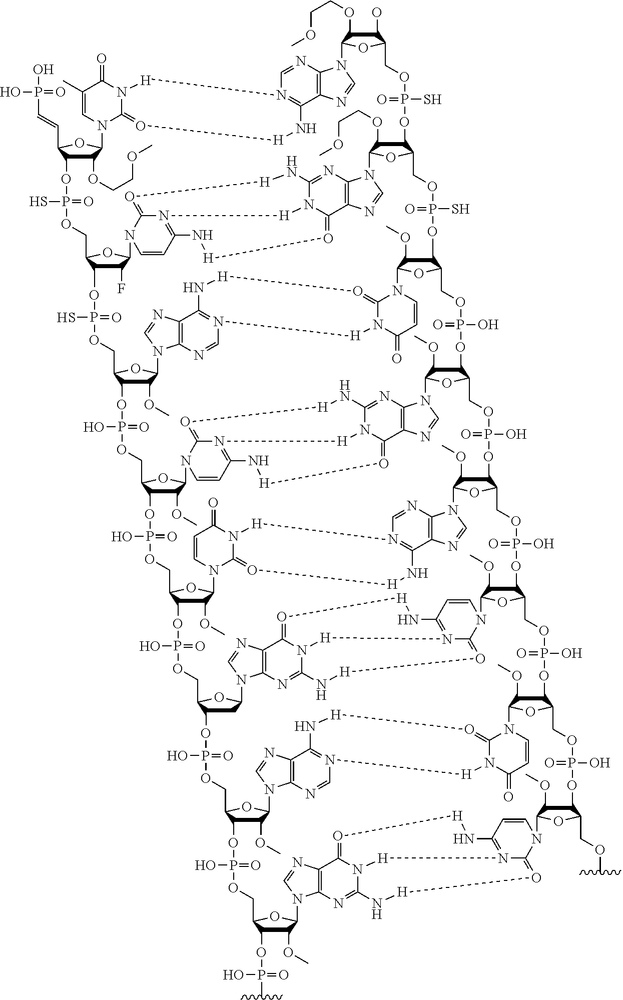

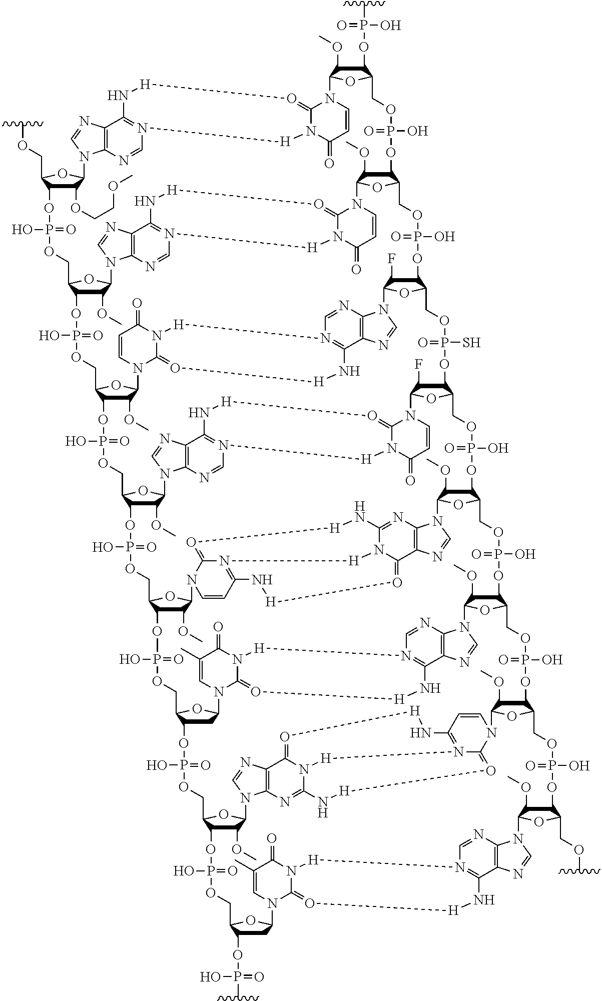

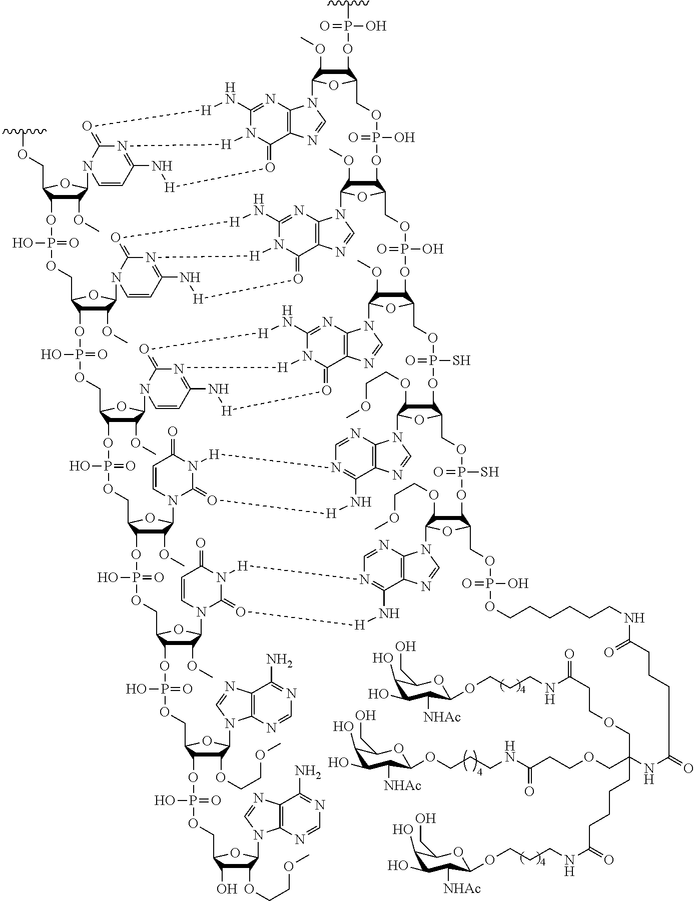

(SEQ ID NO: 28 and SEQ ID NO: 40), or an ion or salt thereof.

In certain embodiments, Compound No. 1754976 is in the form of an anion or a salt thereof, for example, a sodium salt. In certain embodiments, the oligomeric duplex is in anionic form in a solution. In certain embodiments, Compound No. 1754976 is a sodium salt or a potassium salt.

The following chemical structure represents Compound No. 1754976 in sodium solution:

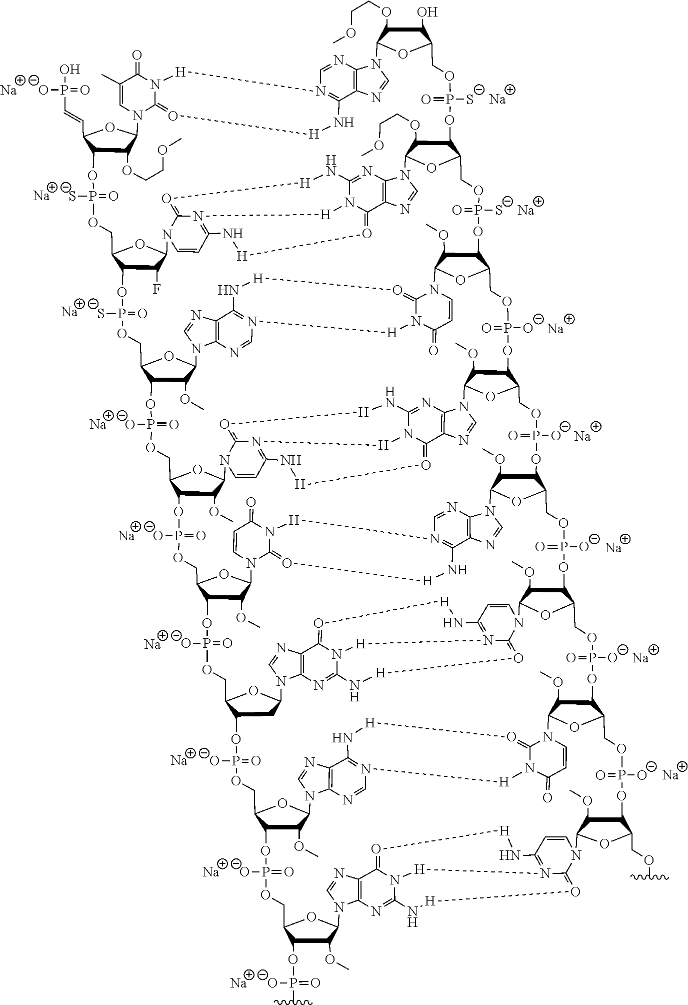

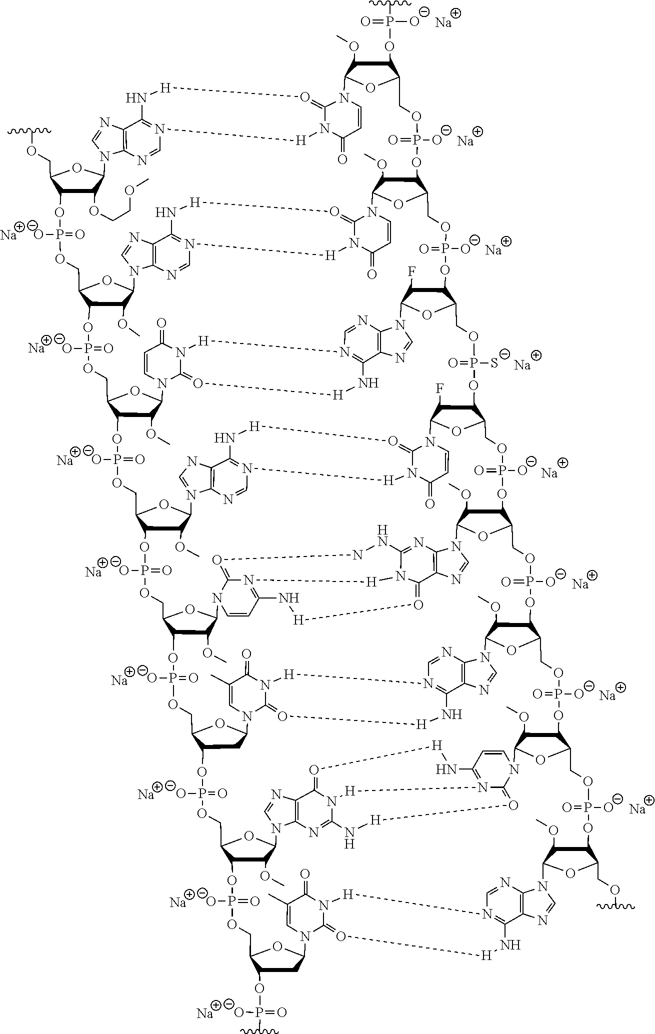

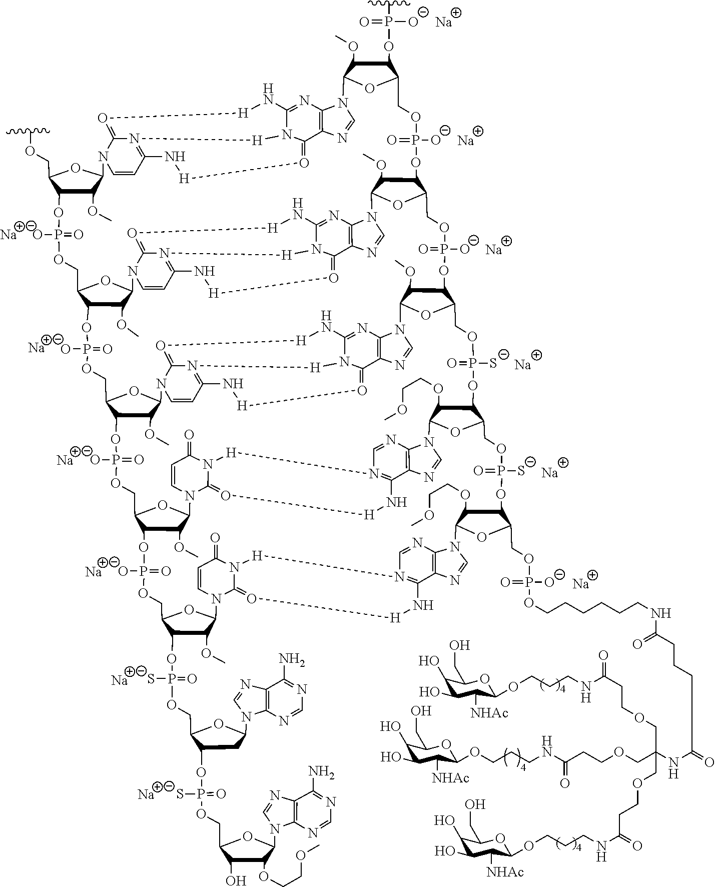

### (SEQ ID NO: 28 and SEQ ID NO: 40).

In certain embodiments, provided herein are oligomeric duplexes comprising or consisting of a first oligomeric compound containing a first modified oligonucleotide and a second oligomeric compound containing a second oligomeric compound as follows.

The first oligomeric compound is Compound No. 1753166, as described in b. above for Compound no. 1754976.

The second oligomeric compound has a nucleobase sequence of (from 5′ to 3′) AAGGGACAGUAUUCUCAGUGA (SEQ ID NO: 6), wherein each of nucleosides 1, 2, 20 and 21 (from 5′ to 3′) comprises a 2′-MOE modified sugar moiety, each of nucleosides 10 and 11 (from 5′ to 3′) comprises a 2′-fluoro modified sugar moiety, and each of nucleosides 3-9, and 12-19 (from 5′ to 3′) comprises a 2′-OMe modified sugar moiety, wherein each of the internucleoside linkages linking nucleosides 1 to 2, 2 to 3, 10 to 11, 19 to 20, and 20 to 21 (from 5′ to 3′) is a phosphorothioate internucleoside linkage and each of the internucleoside linkages linking nucleosides 3 to 4, 4 to 5, 5 to 6, 6 to 7, 7 to 8, 8 to 9, 9 to 10, 11 to 12, 12 to 13, 13 to 14, 14 to 15, 15 to 16, 16 to 17, 17 to 18, and 18 to 19 (from 5′ to 3′) is a phosphodiester linkage.

The second oligomeric compound is represented by the following chemical notation: AₑₛAₑₛG_{yo}G_{yo}G_{yo}A_{yo}C_{yo}A_{yo}G_{yo}U_{fs}A_{fo}U_{yo}U_{yo}C_{yo}U_{yo}C_{yo}A_{yo}G_{yo}U_{ys}GₑₛAₑ (SEQ ID NO: 80); wherein:
A=an adenine nucleobase,
C=a cytosine nucleobase,
G=a guanine nucleobase,
T=a thymine nucleobase,
U=a uracil nucleobase,
e=a 2′-MOE sugar moiety,
f=a 2′-fluoro sugar moiety,
y=a 2′-OMe sugar moiety,
o=a phosphodiester internucleoside linkage, and
s=a phosphorothioate internucleoside linkage.

In certain embodiments, such an oligomeric duplex is represented by the following chemical structure:

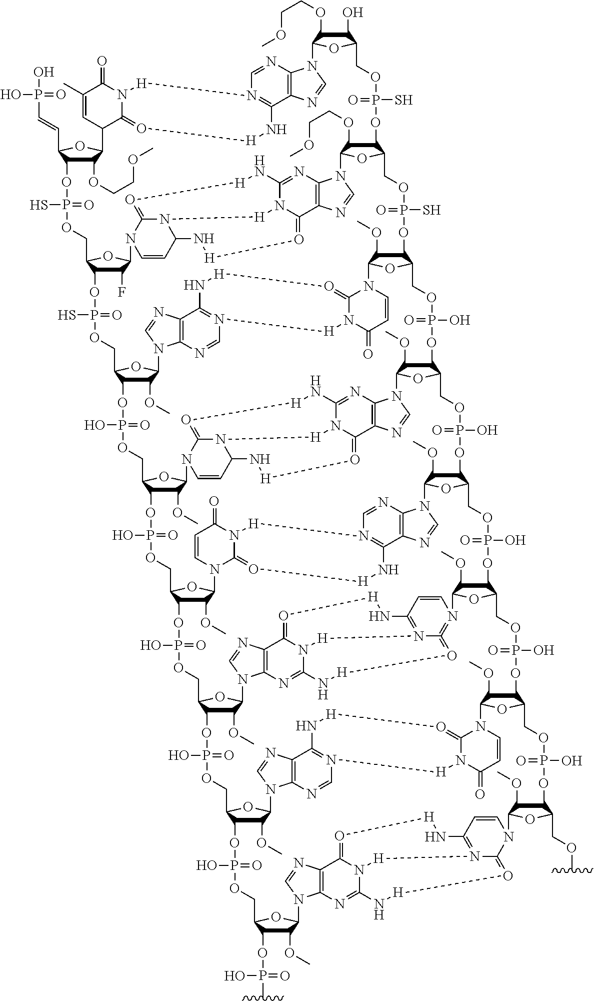

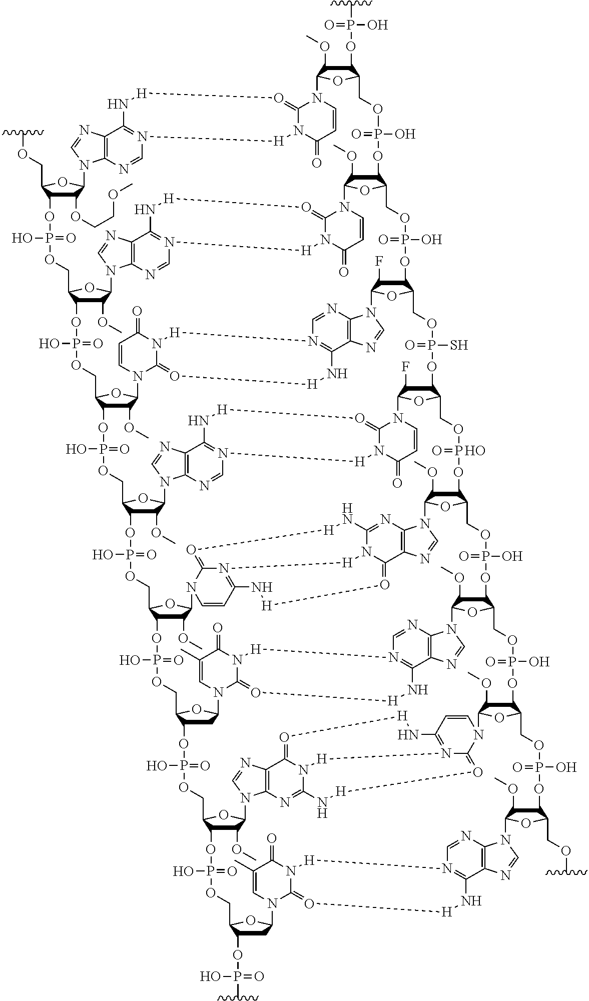

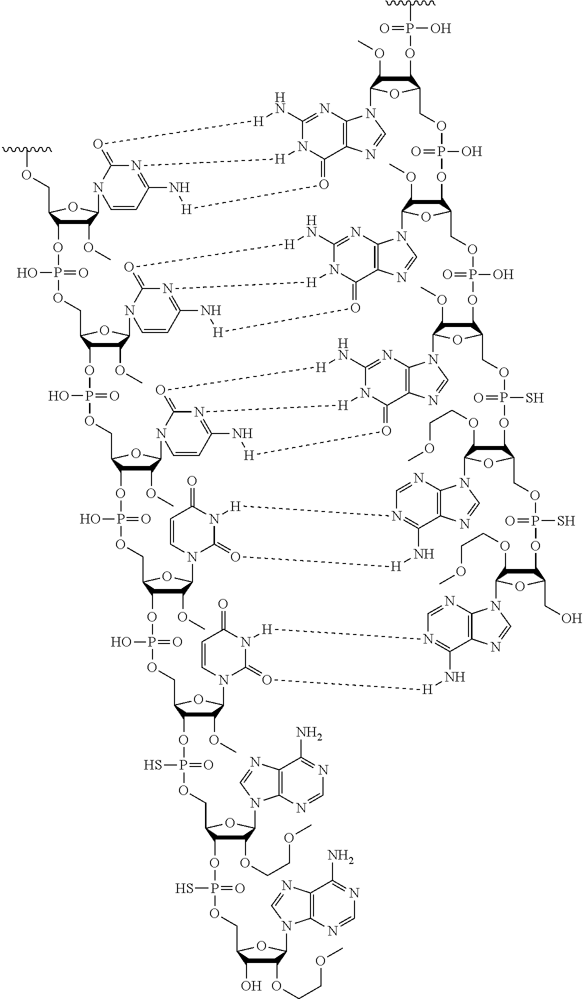

(SEQ ID NO: 28 and SEQ ID NO: 80), or an ion or salt thereof.

In certain embodiments, such an oligomeric duplex is in the form of an anion or a salt thereof, for example, a sodium salt. In certain embodiments, the oligomeric duplex is in anionic form in a solution. In certain embodiments, the oligomeric duplex is a sodium salt or a potassium salt.

The following chemical structure represents such an oligomeric duplex in sodium solution:

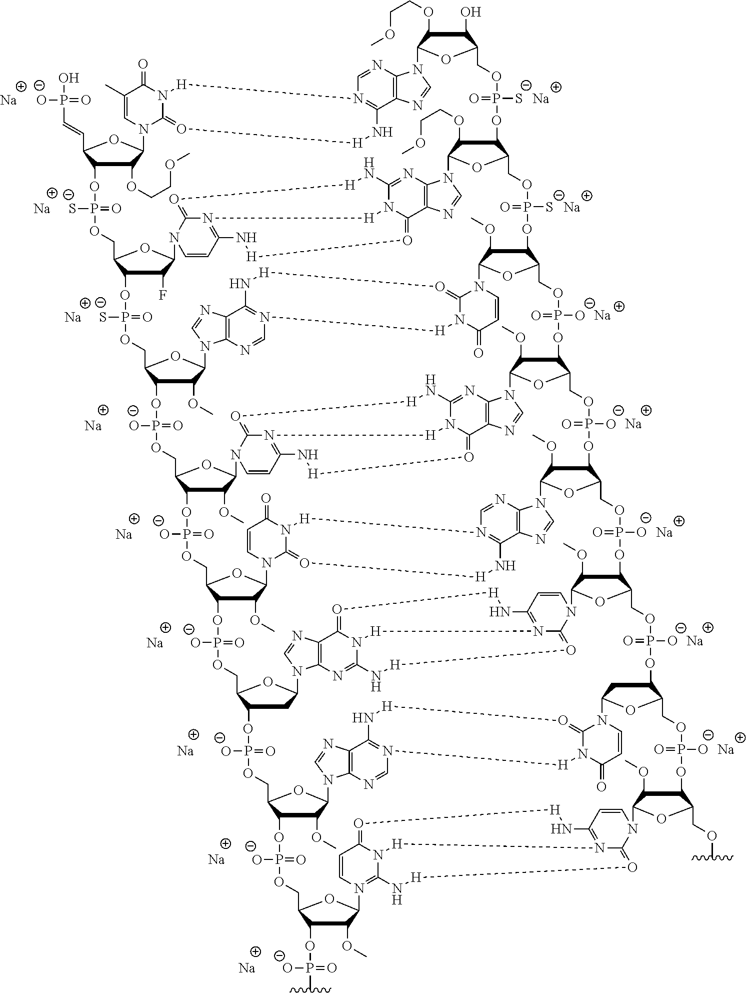

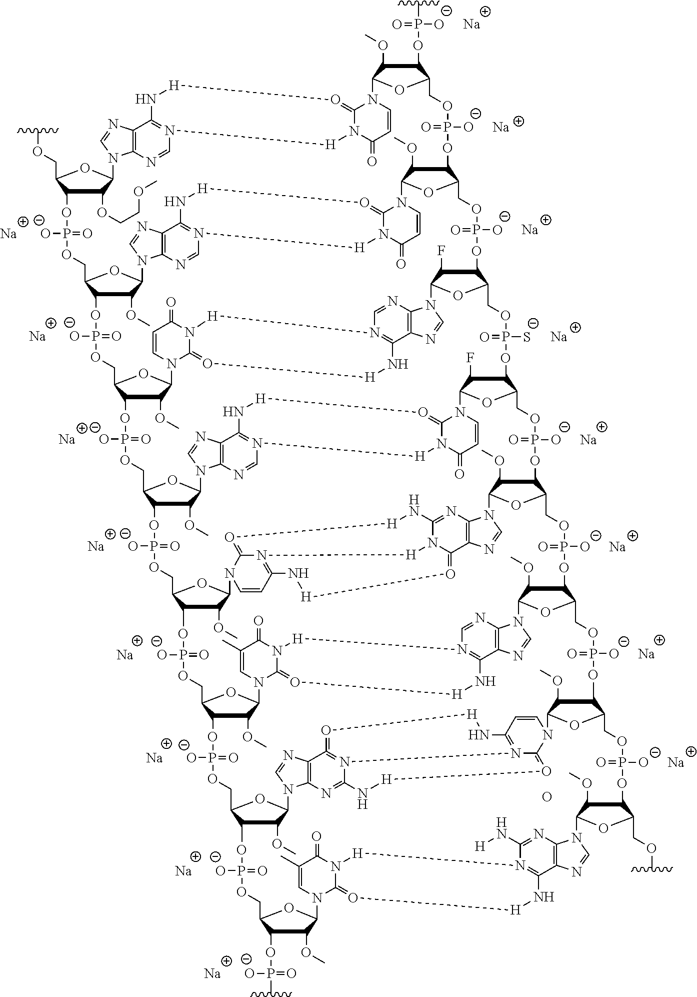

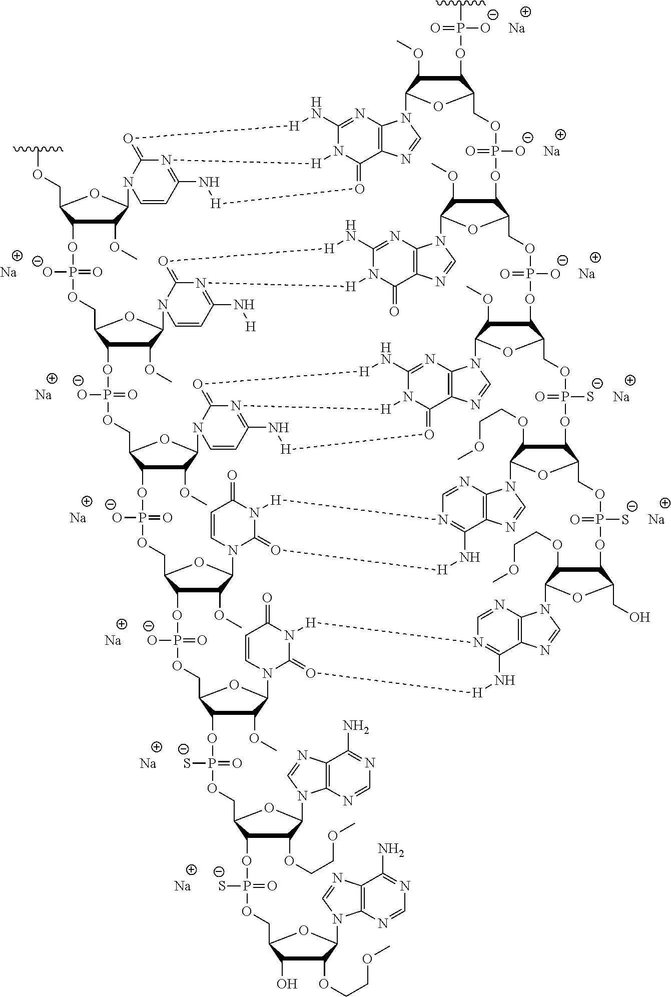

### (SEQ ID NO: 28 and SEQ ID NO: 80).

c. Compound No. 1755063

Provided herein is Compound No. 1755063, which is an oligomeric duplex that consists of a first oligomeric compound containing a first modified oligonucleotide and a second oligomeric compound containing a second modified oligonucleotide attached to a conjugate group as follows.

### First Oligomeric Compound of Compound No. 1755063:

The first modified oligonucleotide of Compound no. 1755063, which is Compound No. 1753167, has a first modified oligonucleotide having a nucleobase sequence of (from 5′ to 3′) TCACUGAGAAUACUGUCCCUUAA (SEQ ID NO: 3), wherein each of nucleosides 1, 9, 22 and 23 (from 5′ to 3′) comprises a 2′-MOE modified sugar moiety, nucleosides 2 (from 5′ to 3′) comprises a 2′-fluoro modified sugar moiety, each of nucleosides 3-5, 7, 8, 10-13, 15 and 17-21 (from 5′ to 3′) comprises a 2′-OMe modified sugar moiety, and each of nucleosides 6, 14, 16 (from 5′ to 3′) is a 2′-deoxynucleoside, wherein each of the internucleoside linkages linking nucleosides 1 to 2, 2 to 3, 6 to 7, 14 to 15, 16 to 17, 21 to 22, and 22 to 23 (from 5′ to 3′) is a phosphorothioate internucleoside linkage and each of the internucleoside linkages linking nucleosides 3 to 4, 4 to 5, 5 to 6, 7 to 8, 8 to 9, 9 to 10, 10 to 11, 11 to 12, 12 to 13, 13 to 14, 15 to 16, 17 to 18, 18 to 19, 19 to 20, and 20 to 21 (from 5′ to 3′) is a phosphodiester linkage, and wherein nucleoside 1 (from 5′ to 3′) is attached to a vinyl phosphonate moiety.

The first modified oligonucleotide of Compound 1755063 is represented by the following chemical notation: VP-TₑₛC_{fs}A_{yo}C_{yo}U_{yo}G_{ds}A_{yo}G_{yo}AₑₒA_{yo}U_{yo}A_{yo}C_{yo}U_{ds}G_{yo}U_{ds}C_{yo}C_{yo}C_{yo}U_{yo}U_{ys}AₑₛAₑ (SEQ ID NO: 29); wherein
A=an adenine nucleobase,
C=a cytosine nucleobase,
G=a guanine nucleobase,
T=a thymine nucleobase,
U=a uracil nucleobase,
d=a 2′-β-D-deoxyribosyl sugar moiety,
e=a 2′-MOE sugar moiety,
f=a 2′-fluoro sugar moiety,
y=a 2′-OMe sugar moiety,
o=a phosphodiester internucleoside linkage,
s=a phosphorothioate internucleoside linkage, and
VP=a 5′ vinyl phosphonate moiety.

### Second Oligomeric Compound of Compound No. 1755063:

The second oligomeric compound of Compound No. 1755063, which is Compound No. 1718715, has a nucleobase sequence of (from 5′ to 3′) AAGGGACAGUAUUCUCAGUGA (SEQ ID NO: 6), wherein each of nucleosides 1, 2, 20 and 21 (from 5′ to 3′) comprises a 2′-MOE modified sugar moiety, each of nucleosides 10 and 11 (from 5′ to 3′) comprises a 2′-fluoro modified sugar moiety, and each of nucleosides 3-9, and 12-19 (from 5′ to 3′) comprises a 2′-OMe modified sugar moiety, wherein each of the internucleoside linkages linking nucleosides 1 to 2, 2 to 3, 19 to 20, and 20 to 21 (from 5′ to 3′) is a phosphorothioate internucleoside linkage and each of the internucleoside linkages linking nucleosides 3 to 4, 4 to 5, 5 to 6, 6 to 7, 7 to 8, 8 to 9, 9 to 10, 10 to 11, 11 to 12, 12 to 13, 13 to 14, 14 to 15, 15 to 16, 16 to 17, 17 to 18, and 18 to 19 (from 5′ to 3′) is a phosphodiester linkage, and wherein nucleoside 1 (from 5′ to 3′) is attached is attached through the 5′-OH of the oligonucleotide to a conjugate group comprising [THA-GalNAc].

The second oligomeric compound of Compound 1755063 is represented by the following chemical notation: [THA-GalNAc]-AₑₛAₑₛG_{yo}G_{yo}G_{yo}A_{yo}C_{yo}A_{yo}G_{yo}U_{fo}A_{fo}U_{yo}U_{yo}C_{yo}U_{yo}C_{yo}A_{yo}G_{yo}U_{ys}GₑₛAₑ (SEQ ID NO: 39); wherein:

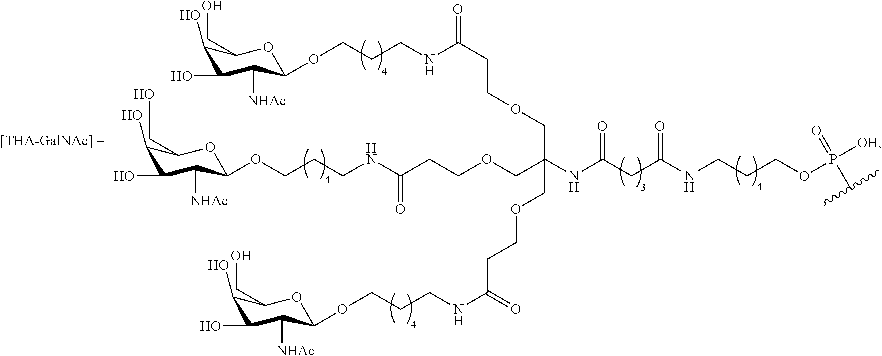

A=an adenine nucleobase,
C=a cytosine nucleobase,
G=a guanine nucleobase,
T=a thymine nucleobase,
U=a uracil nucleobase,
e=a 2′-MOE sugar moiety,
f=a 2′-fluoro sugar moiety,
y=a 2′-OMe sugar moiety,
o=a phosphodiester internucleoside linkage, and
s=a phosphorothioate internucleoside linkage.

The following chemical structure is one structural representation of Compound No. 1755063:

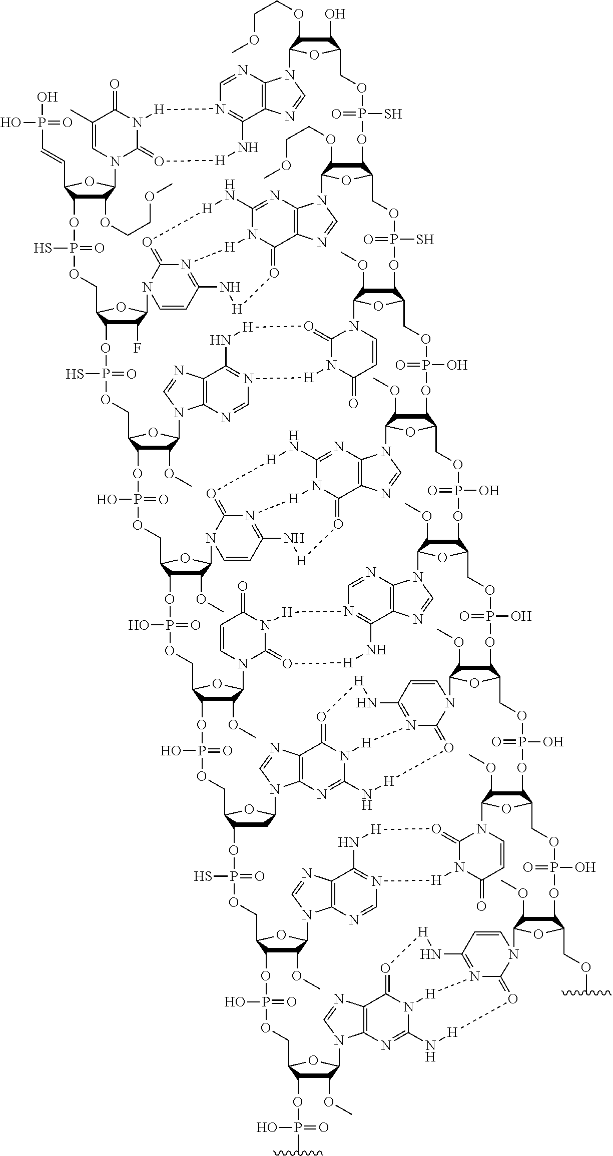

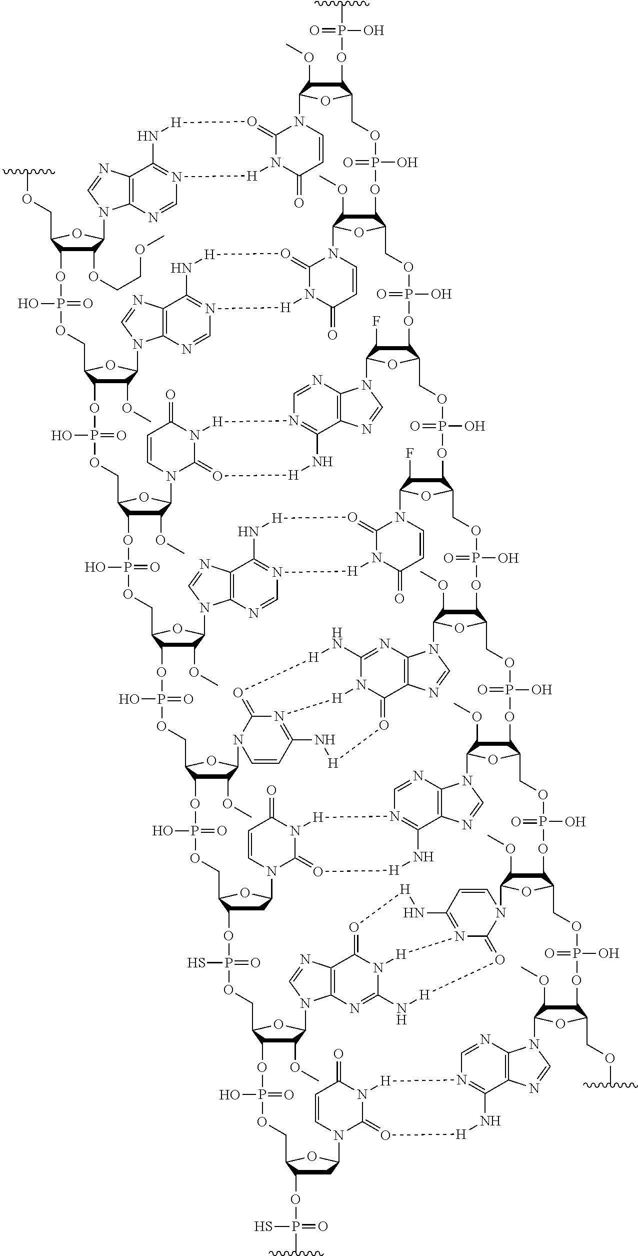

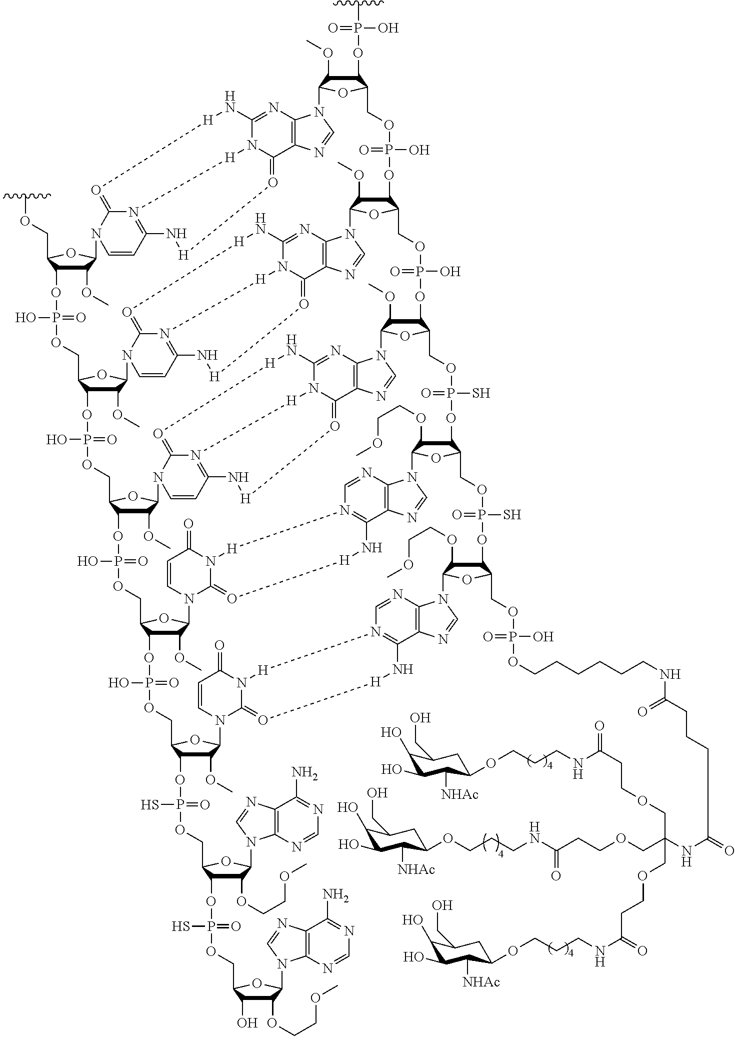

(SEQ ID NO: 29 and SEQ ID NO: 39), or an ion or salt thereof.

In certain embodiments, Compound No. 1755063 is in the form of an anion or a salt thereof, for example, the oligomeric duplex may be in the form of a sodium salt. In certain embodiments, the oligomeric duplex is in anionic form in a solution. In certain embodiments, Compound No. 1755063 is a sodium salt or a potassium salt.

The following chemical structure represents Compound No. 1755063 in sodium solution:

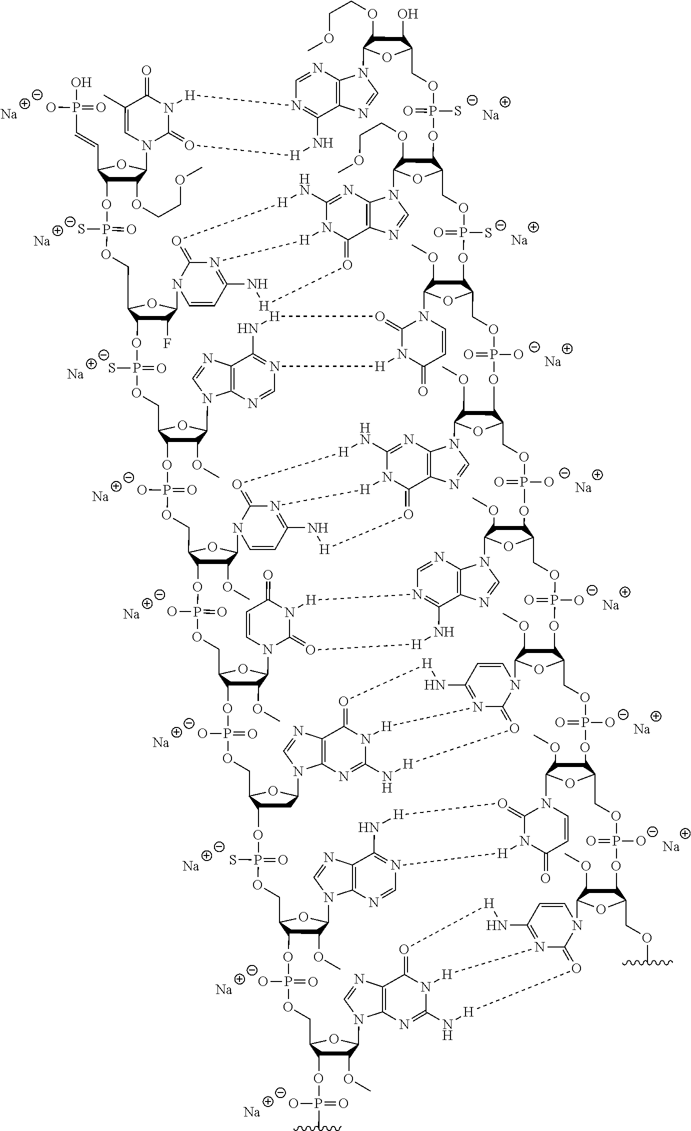

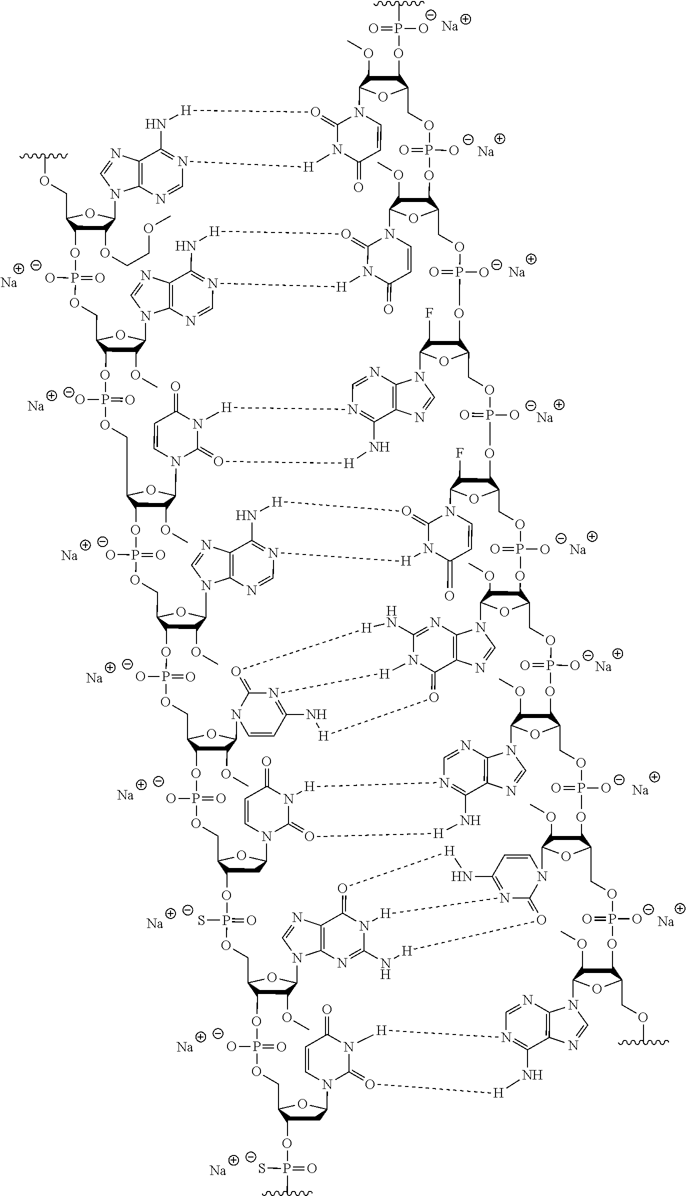

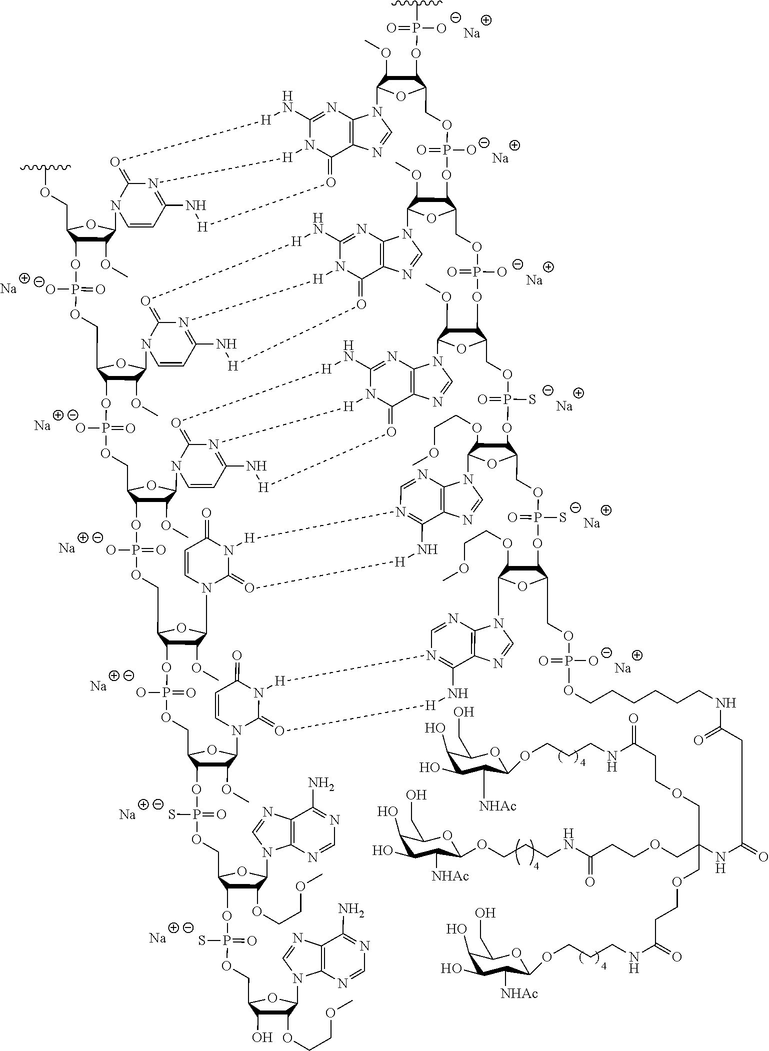

### (SEQ ID NO: 29 and SEQ ID NO: 39).

In certain embodiments, provided herein are oligomeric duplexes comprising or consisting of a first oligomeric compound containing a first modified oligonucleotide and a second oligomeric compound containing a second modified oligonucleotide as follows.

The first modified oligonucleotide is Compound No. 1753167 (SEQ ID NO: 29), as described in c. above for Compound no. 1755063.

The second oligomeric compound has a nucleobase sequence of (from 5′ to 3′) AAGGGACAGUAUUCUCAGUGA (SEQ ID NO: 6), wherein each of nucleosides 1, 2, 20 and 21 (from 5′ to 3′) comprises a 2′-MOE modified sugar moiety, each of nucleosides 10 and 11 (from 5′ to 3′) comprises a 2′-fluoro modified sugar moiety, and each of nucleosides 3-9, and 12-19 (from 5′ to 3′) comprises a 2′-OMe modified sugar moiety, wherein each of the internucleoside linkages linking nucleosides 1 to 2, 2 to 3, 19 to 20, and 20 to 21 (from 5′ to 3′) is a phosphorothioate internucleoside linkage and each of the internucleoside linkages linking nucleosides 3 to 4, 4 to 5, 5 to 6, 6 to 7, 7 to 8, 8 to 9, 9 to 10, 10 to 11, 11 to 12, 12 to 13, 13 to 14, 14 to 15, 15 to 16, 16 to 17, 17 to 18, and 18 to 19 (from 5′ to 3′) is a phosphodiester linkage.

The second oligomeric compound is represented by the following chemical notation: AₑₛAₑₛG_{yo}G_{yo}G_{yo}A_{yo}C_{yo}A_{yo}G_{yo}U_{fo}A_{fo}U_{yo}U_{yo}C_{yo}U_{yo}C_{yo}A_{yo}G_{yo}U_{ys}GₑₛAₑ (SEQ ID NO: 79); wherein:
A=an adenine nucleobase,
C=a cytosine nucleobase,
G=a guanine nucleobase,
T=a thymine nucleobase,
U=a uracil nucleobase,
e=a 2′-MOE sugar moiety,
f=a 2′-fluoro sugar moiety,
y=a 2′-OMe sugar moiety,
o=a phosphodiester internucleoside linkage, and
s=a phosphorothioate internucleoside linkage.

In certain embodiments, such an oligomeric duplex is represented by the following chemical structure:

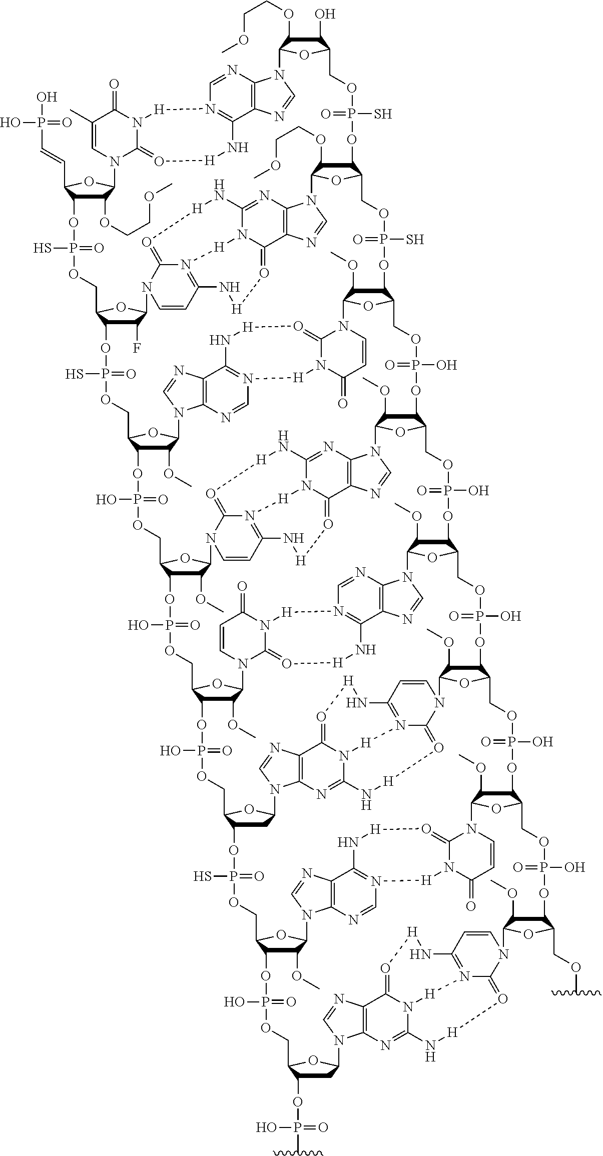

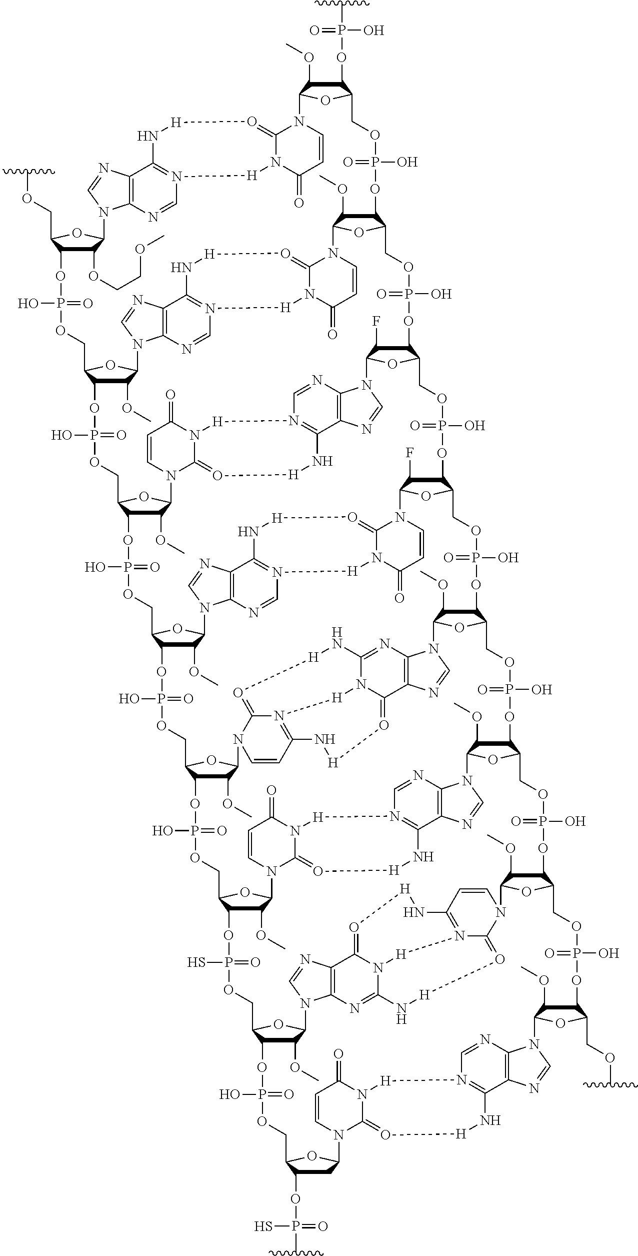

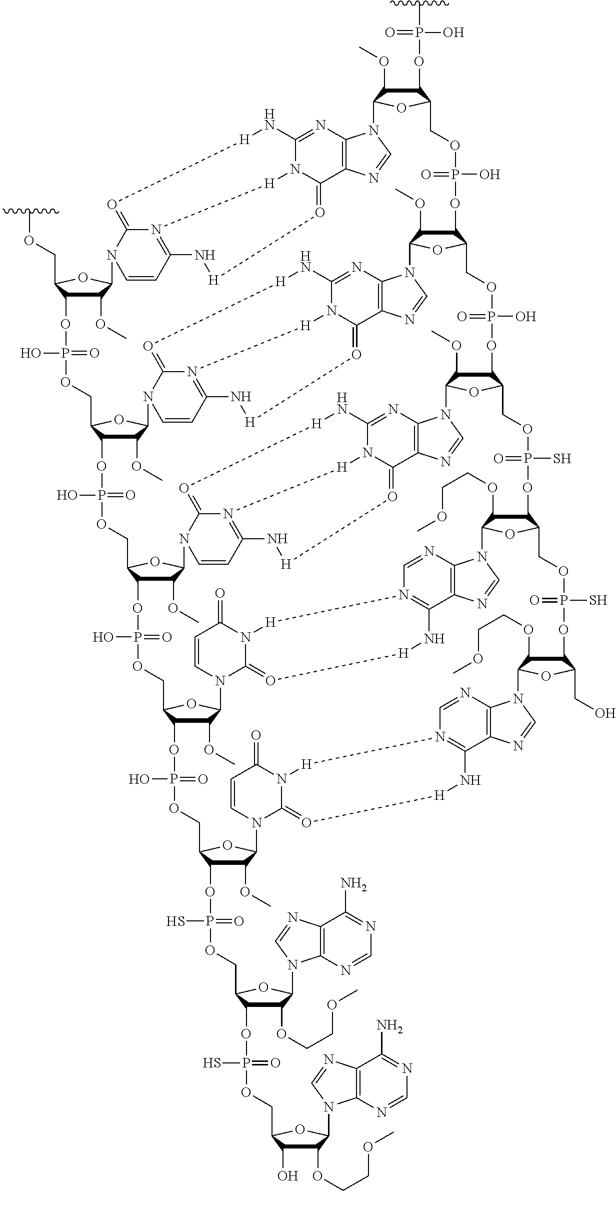

(SEQ ID NO: 29 and SEQ ID NO: 79), or an ion or salt thereof.

In certain embodiments, such an oligomeric duplex is in the form of an anion or a salt thereof, for example, a sodium salt. In certain embodiments, the oligomeric duplex is in anionic form in a solution. In certain embodiments, the oligomeric duplex is a sodium salt or a potassium salt.

The following chemical structure represents such an oligomeric duplex in sodium solution:

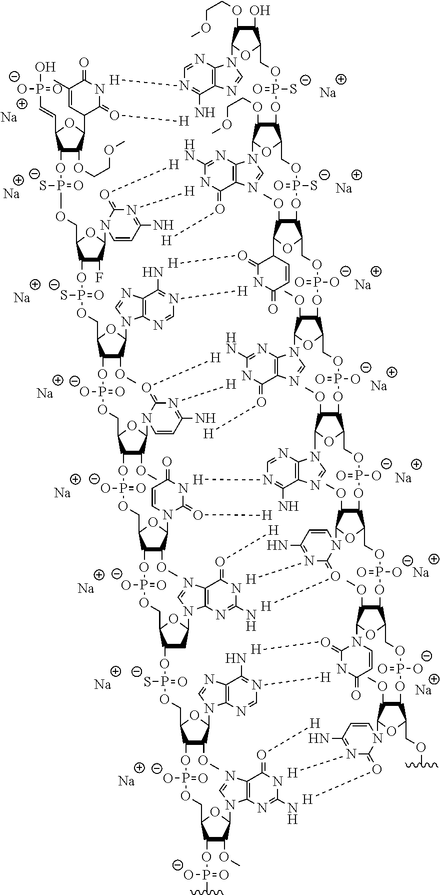

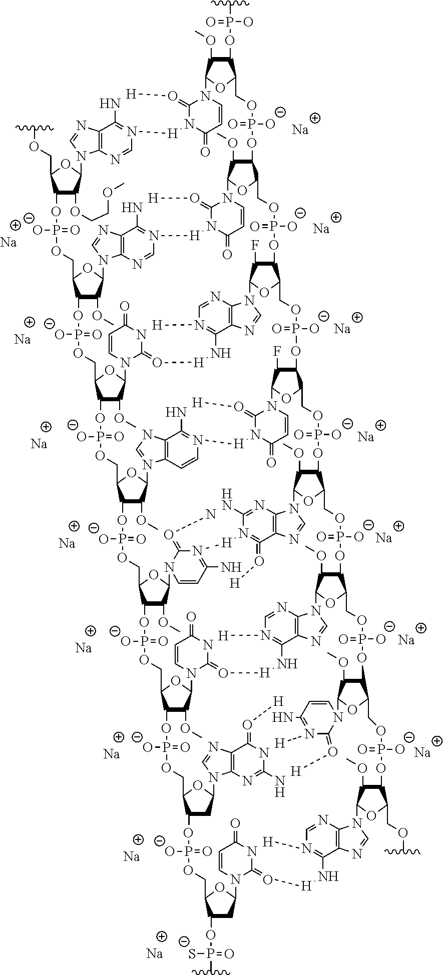

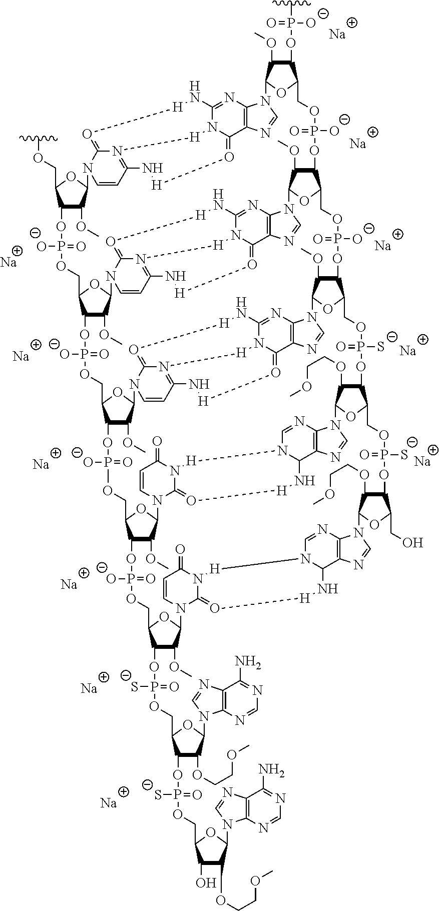

### (SEQ ID NO: 29 and SEQ ID NO: 79).

d. Compound No. 1755069

Provided herein is Compound No. 1755069, which is an oligomeric duplex that consists of a first oligomeric compound containing a first modified oligonucleotide and a second oligomeric compound containing a second modified oligonucleotide attached to a conjugate group as follows.

### First Oligomeric Compound of Compound No. 1755069:

The first oligomeric compound of Compound no. 1755069, which is Compound No. 1752680, has a first modified oligonucleotide having a nucleobase sequence of (from 5′ to 3′) TCACUGAGAAUACUGUCCCUUAA (SEQ ID NO: 3), wherein each of nucleosides 1, 9, 10, 22 and 23 (from 5′ to 3′) comprises a 2′-MOE modified sugar moiety, nucleoside 2 (from 5′ to 3′) comprises a 2′-fluoro modified sugar moiety, each of nucleosides 3-5, 7, 8, 11-13, 15 and 17-21 (from 5′ to 3′) comprises a 2′-OMe modified sugar moiety, each of nucleosides 6, 14, and 16 (from 5′ to 3′) is a 2′-deoxynucleoside, wherein each of the internucleoside linkages linking nucleosides 1 to 2, 2 to 3, 21 to 22, and 22 to 23 (from 5′ to 3′) is a phosphorothioate internucleoside linkage and each of the internucleoside linkages linking nucleosides 3 to 4, 4 to 5, 5 to 6, 6 to 7, 7 to 8, 8 to 9, 9 to 10, 10 to 11, 11 to 12, 12 to 13, 13 to 14, 14 to 15, 15 to 16, 16 to 17, 17 to 18, 18 to 19, 19 to 20, and 20 to 21 (from 5′ to 3′) is a phosphodiester linkage, and wherein nucleoside 1 (from 5′ to 3′) is attached to a vinyl phosphonate moiety.

The first oligomeric compound of Compound 1755069 is represented by the following chemical notation: VP-TₑₛC_{fs}A_{yo}C_{yo}U_{yo}G_{do}A_{yo}G_{yo}AₑₒAₑₒU_{yo}A_{yo}C_{yo}U_{do}G_{yo}U_{do}C_{yo}C_{yo}C_{yo}U_{yo}U_{ys}AₑₛAₑ (SEQ ID NO: 26); wherein:
A=an adenine nucleobase,
C=a cytosine nucleobase,
G=a guanine nucleobase,
T=a thymine nucleobase,
U=a uracil nucleobase,
d=a 2′-β-D-deoxyribosyl sugar moiety,
e=a 2′-MOE sugar moiety,
f=a 2′-fluoro sugar moiety,
y=a 2′-OMe sugar moiety,
o=a phosphodiester internucleoside linkage,
s=a phosphorothioate internucleoside linkage, and
VP=a 5′ vinyl phosphonate moiety.

### Second Oligomeric Compound of Compound No. 1755069:

The second oligomeric compound of Compound No. 1755069, which is Compound No. 1735443, has a nucleobase sequence of (from 5′ to 3′) AAGGGACAGUAUUCUCAGUGA (SEQ ID NO: 6), wherein each of nucleosides 1, 2, 20 and 21 (from 5′ to 3′) comprises a 2′-MOE modified sugar moiety, each of nucleosides 10 and 11 (from 5′ to 3′) comprises a 2′-fluoro modified sugar moiety, and each of nucleosides 3-9, and 12-19 (from 5′ to 3′) comprises a 2′-OMe modified sugar moiety, wherein each of the internucleoside linkages linking nucleosides 1 to 2, 2 to 3, 10 to 11, 19 to 20, and 20 to 21 (from 5′ to 3′) is a phosphorothioate internucleoside linkage and each of the internucleoside linkages linking nucleosides 3 to 4, 4 to 5, 5 to 6, 6 to 7, 7 to 8, 8 to 9, 9 to 10, 11 to 12, 12 to 13, 13 to 14, 14 to 15, 15 to 16, 16 to 17, 17 to 18, and 18 to 19 (from 5′ to 3′) is a phosphodiester linkage, and wherein nucleoside 1 (from 5′ to 3′) is attached is attached through the 5′-OH of the oligonucleotide to a conjugate group comprising [THA-GalNAc].

The second oligomeric compound of Compound 1755069 is represented by the following chemical notation: [THA-GalNAc]-AₑₛAₑₛG_{yo}G_{yo}G_{yo}A_{yo}C_{yo}A_{yo}G_{yo}U_{fs}A_{fo}U_{yo}U_{yo}C_{yo}U_{yo}C_{yo}A_{yo}G_{yo}U_{ys}GₑₛAₑ (SEQ ID NO: 40); wherein:

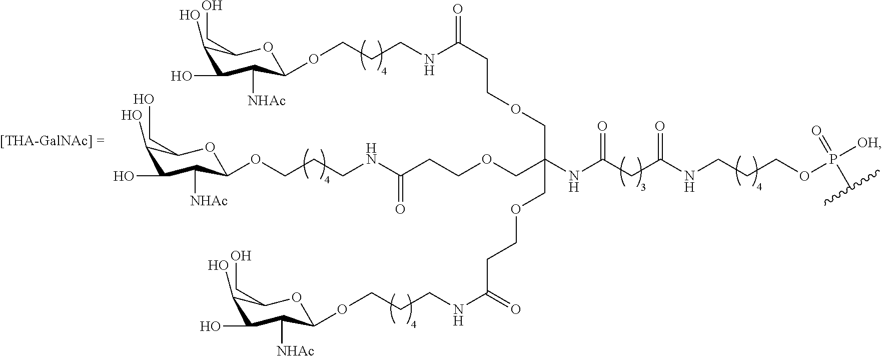

A=an adenine nucleobase,
C=a cytosine nucleobase,
G=a guanine nucleobase,
T=a thymine nucleobase,
U=a uracil nucleobase,
e=a 2′-MOE sugar moiety,
f=a 2′-fluoro sugar moiety,
y=a 2′-OMe sugar moiety,
o=a phosphodiester internucleoside linkage, and
s=a phosphorothioate internucleoside linkage.

The following chemical structure is one structural representation of Compound No. 1755069:

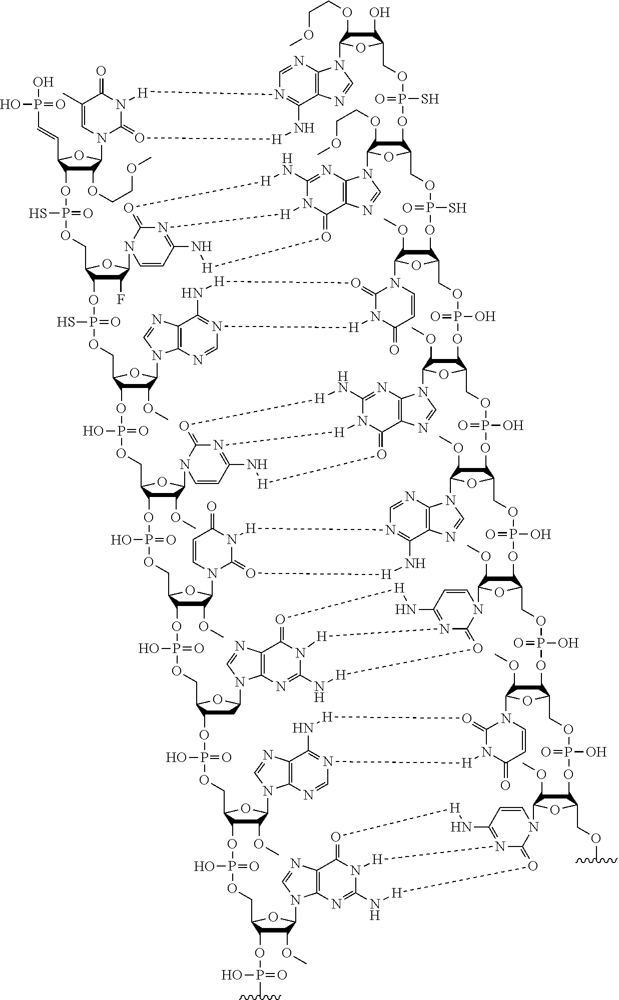

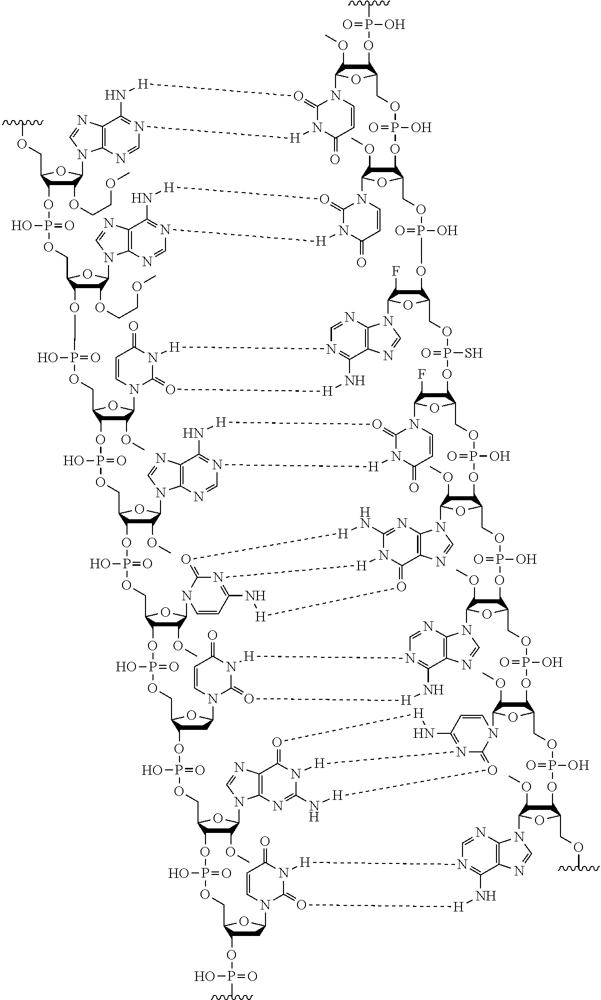

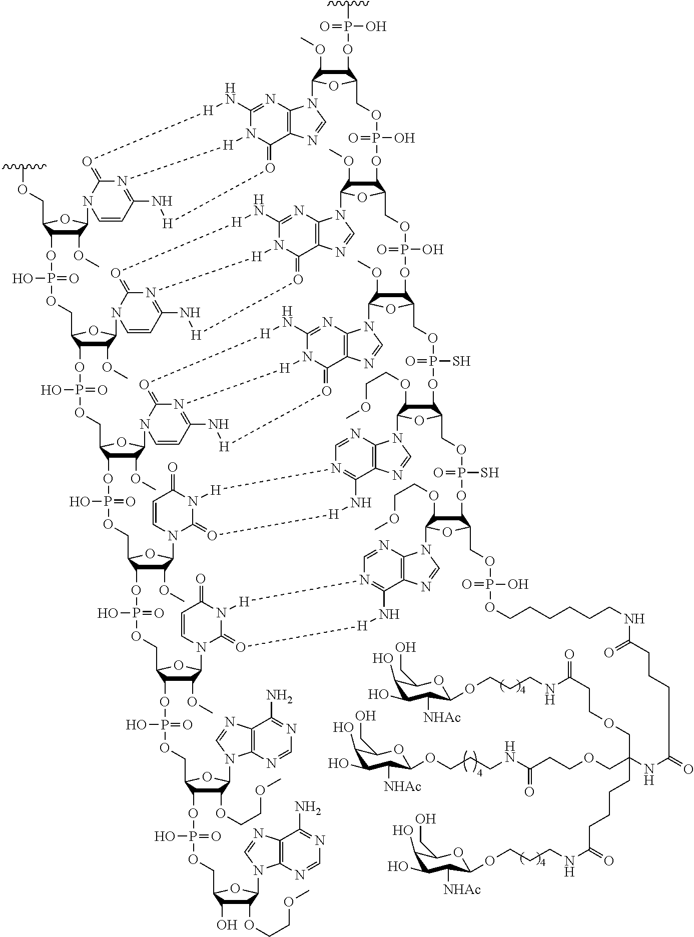

(SEQ ID NO: 26 and SEQ ID NO: 40), or an ion or salt thereof.

In certain embodiments, Compound No. 1755069 is in the form of an anion or a salt thereof, for example, a sodium salt. In certain embodiments, the oligomeric duplex is in anionic form in a solution. In certain embodiments, Compound No. 1755069 is a sodium salt or a potassium salt.

The following chemical structure represents Compound No. 1755069 in sodium solution:

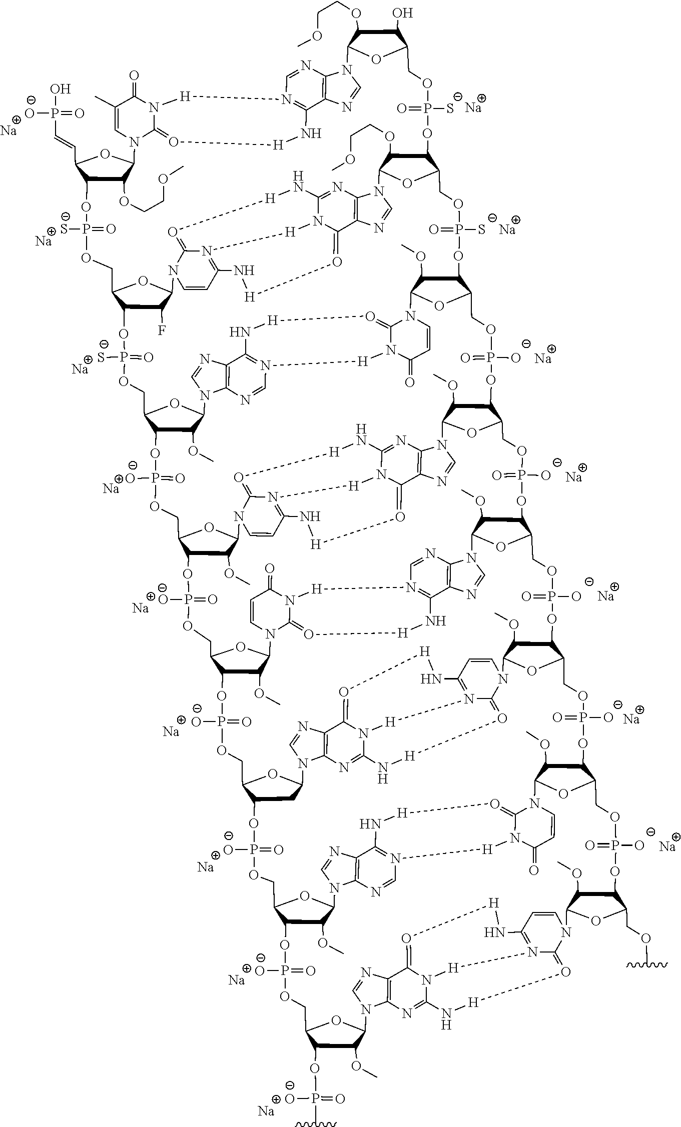

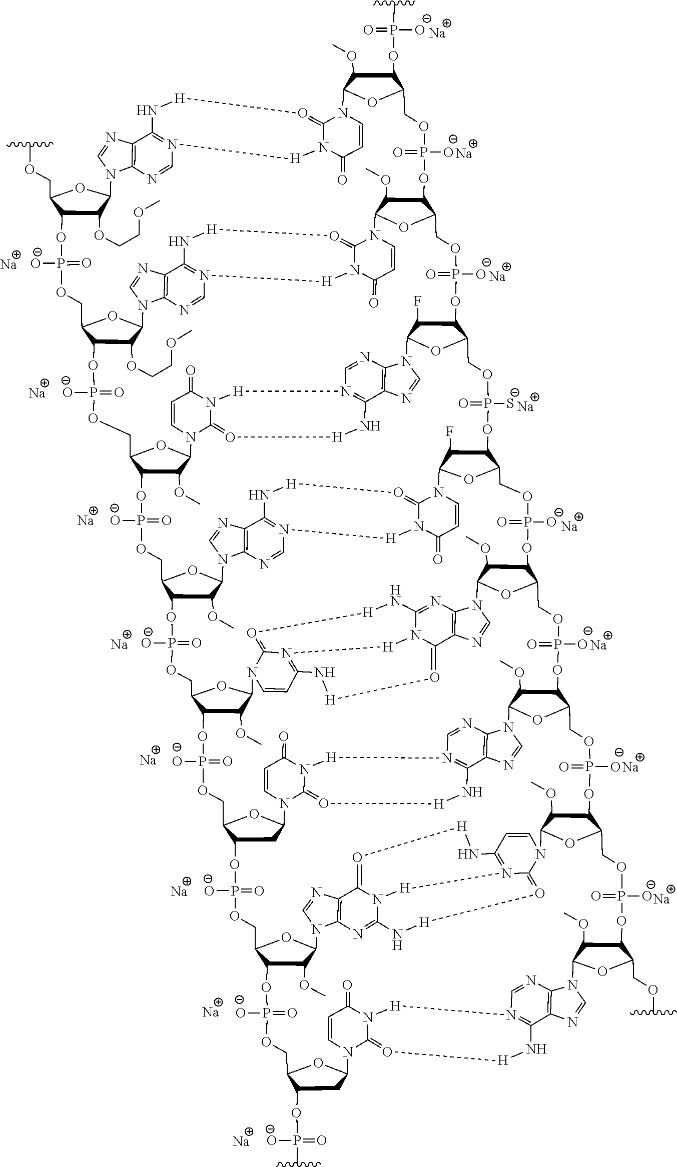

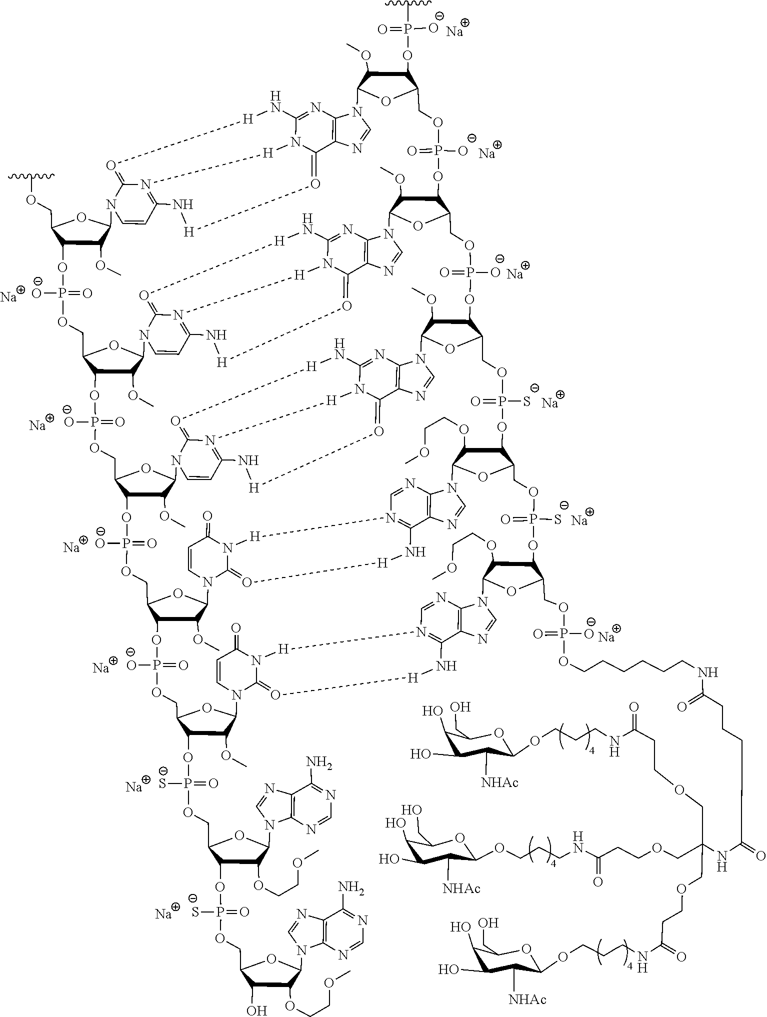

### (SEQ ID NO: 26 and SEQ ID NO: 40).

In certain embodiments, provided herein are oligomeric duplexes comprising or consisting of a first oligomeric compound containing a first modified oligonucleotide and a second oligomeric compound containing a second modified oligonucleotide as follows.

The first modified oligonucleotide is Compound No. 1752680, as described in a. above for Compound no. 1755069.

The second oligomeric compound has a nucleobase sequence of (from 5′ to 3′) AAGGGACAGUAUUCUCAGUGA (SEQ ID NO: 6), wherein each of nucleosides 1, 2, 20 and 21 (from 5′ to 3′) comprises a 2′-MOE modified sugar moiety, each of nucleosides 10 and 11 (from 5′ to 3′) comprises a 2′-fluoro modified sugar moiety, and each of nucleosides 3-9, and 12-19 (from 5′ to 3′) comprises a 2′-OMe modified sugar moiety, wherein each of the internucleoside linkages linking nucleosides 1 to 2, 2 to 3, 10 to 11, 19 to 20, and 20 to 21 (from 5′ to 3′) is a phosphorothioate internucleoside linkage and each of the internucleoside linkages linking nucleosides 3 to 4, 4 to 5, 5 to 6, 6 to 7, 7 to 8, 8 to 9, 9 to 10, 11 to 12, 12 to 13, 13 to 14, 14 to 15, 15 to 16, 16 to 17, 17 to 18, and 18 to 19 (from 5′ to 3′) is a phosphodiester linkage.

The second oligomeric compound is represented by the following chemical notation: AₑₛAₑₛG_{yo}G_{yo}G_{yo}A_{yo}C_{yo}A_{yo}G_{yo}U_{fs}A_{fo}U_{yo}U_{yo}C_{yo}U_{yo}C_{yo}A_{yo}G_{yo}U_{ys}GₑₛAₑ (SEQ ID NO: 80); wherein:
A=an adenine nucleobase,
C=a cytosine nucleobase,
G=a guanine nucleobase,
T=a thymine nucleobase,
U=a uracil nucleobase,
e=a 2′-MOE sugar moiety,
f=a 2′-fluoro sugar moiety,
y=a 2′-OMe sugar moiety,
o=a phosphodiester internucleoside linkage, and
s=a phosphorothioate internucleoside linkage.

In certain embodiments, such an oligomeric duplex is represented by the following chemical structure:

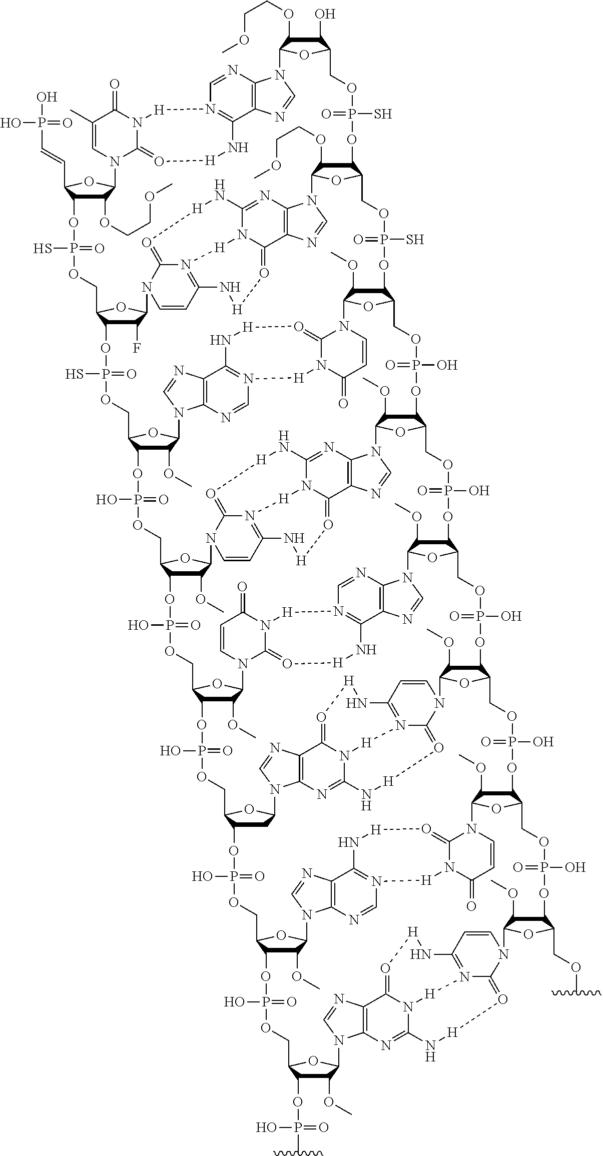

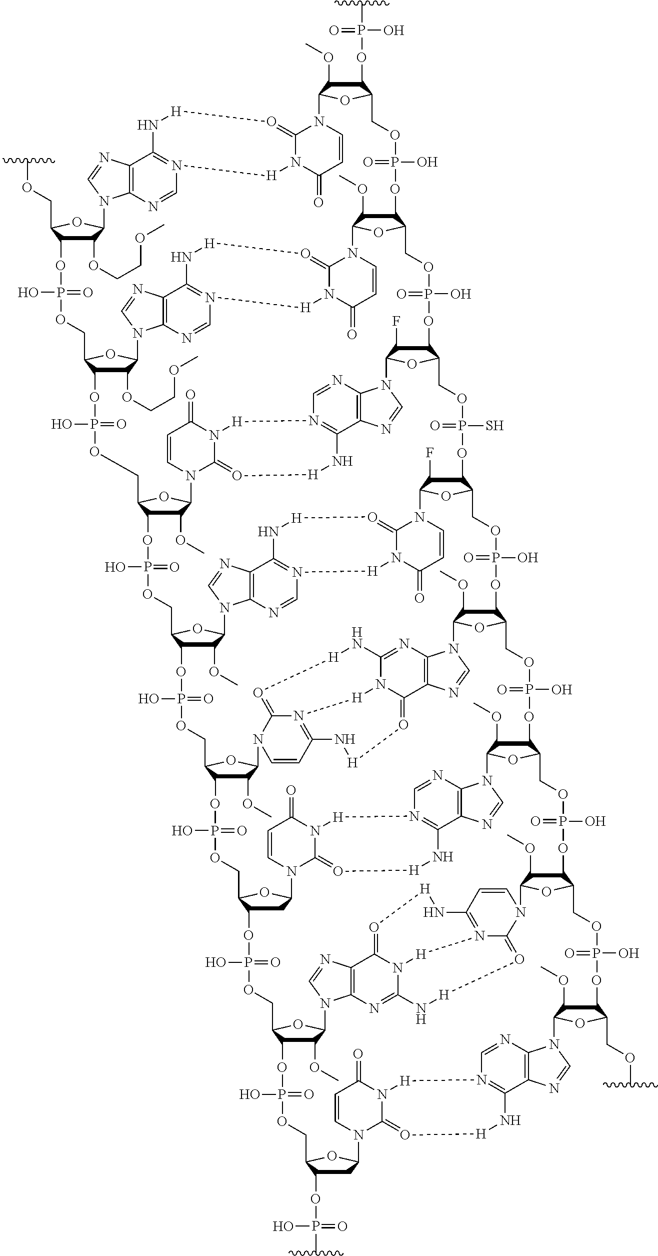

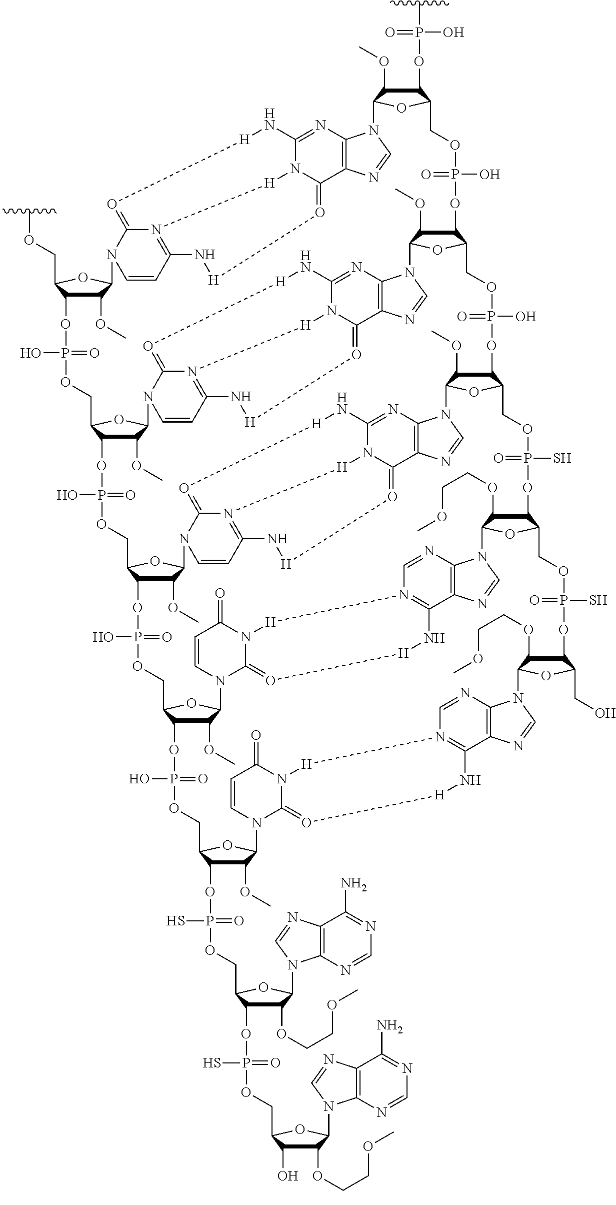

(SEQ ID NO: 26 and SEQ ID NO: 80), or an ion or salt thereof.

In certain embodiments, such an oligomeric duplex is in the form of an anion or a salt thereof, for example, a sodium salt. In certain embodiments, the oligomeric duplex is in anionic form in a solution. In certain embodiments, the oligomeric duplex is a sodium salt or a potassium salt.

The following chemical structure represents such an oligomeric duplex in sodium solution:

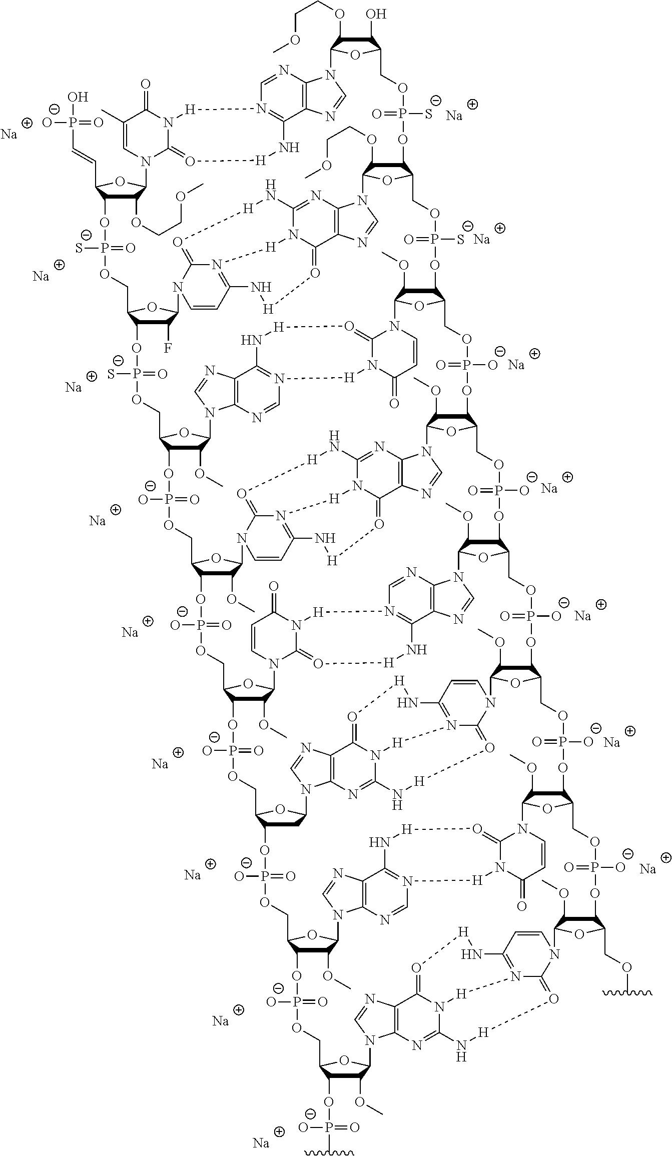

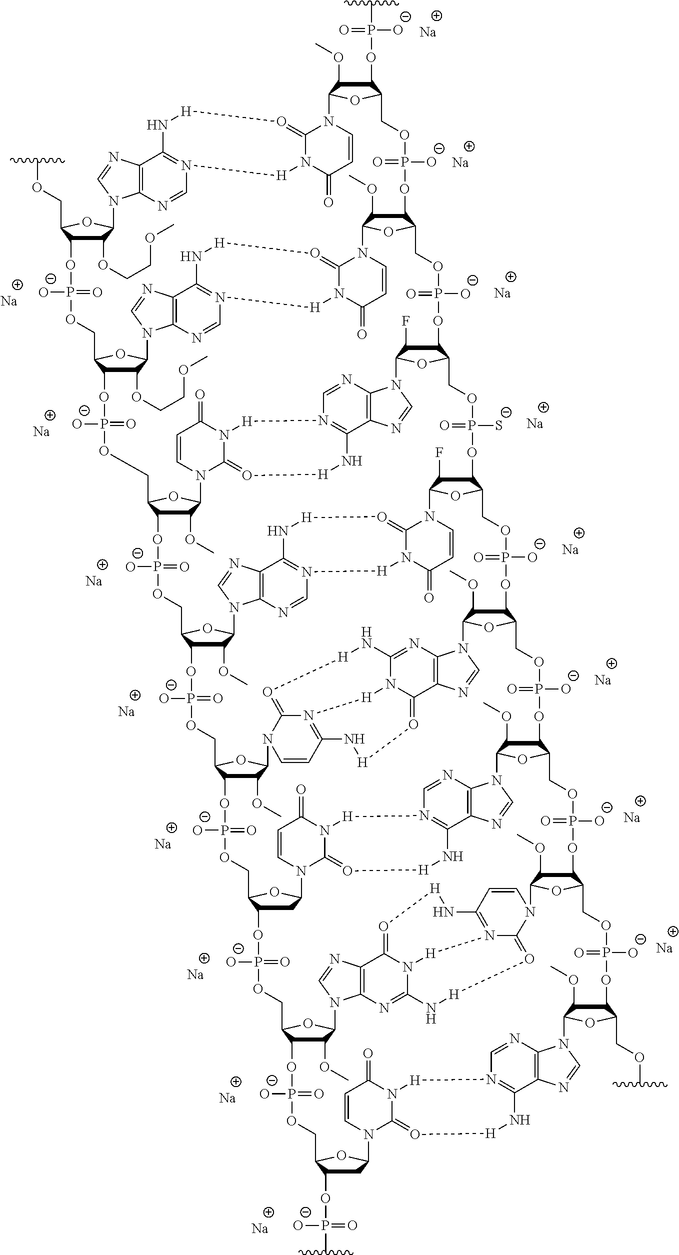

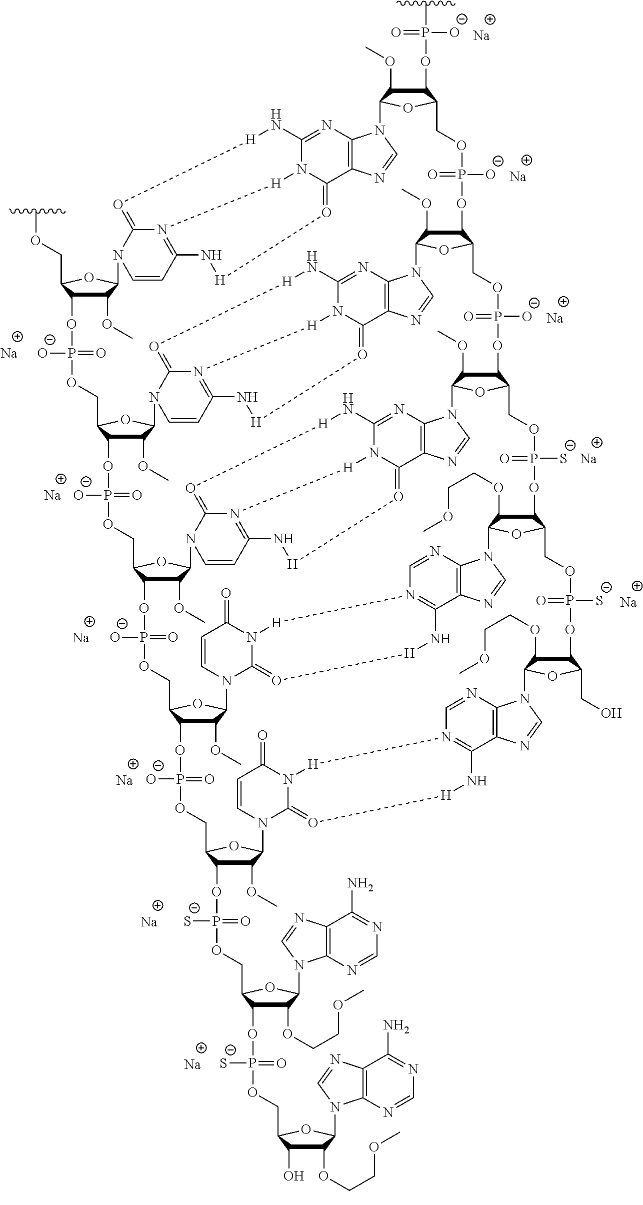

### (SEQ ID NO: 26 and SEQ ID NO: 80).

e. Compound No. 1755072

Provided herein is Compound No. 1755072, which is an oligomeric duplex that consists of a first oligomeric compound containing a first modified oligonucleotide and a second oligomeric compound containing a second modified oligonucleotide attached to a conjugate group as follows.

### First Oligomeric Compound of Compound No. 1755072:

The first oligomeric compound of Compound No. 1755072, which is Compound No. 1753167, has a first modified oligonucleotide having a nucleobase sequence of (from 5′ to 3′) TCACUGAGAAUACUGUCCCUUAA (SEQ ID NO: 3), wherein each of nucleosides 1, 9, 22 and 23 (from 5′ to 3′) comprises a 2′-MOE modified sugar moiety, nucleosides 2 (from 5′ to 3′) comprises a 2′-fluoro modified sugar moiety, each of nucleosides 3-5, 7, 8, 10-13, 15 and 17-21 (from 5′ to 3′) comprises a 2′-OMe modified sugar moiety, and each of nucleosides 6, 14, 16 (from 5′ to 3′) is a 2′-deoxynucleoside, wherein each of the internucleoside linkages linking nucleosides 1 to 2, 2 to 3, 6 to 7, 14 to 15, 16 to 17, 21 to 22, and 22 to 23 (from 5′ to 3′) is a phosphorothioate internucleoside linkage and each of the internucleoside linkages linking nucleosides 3 to 4, 4 to 5, 5 to 6, 7 to 8, 8 to 9, 9 to 10, 10 to 11, 11 to 12, 12 to 13, 13 to 14, 15 to 16, 17 to 18, 18 to 19, 19 to 20, and 20 to 21 (from 5′ to 3′) is a phosphodiester linkage, and wherein nucleoside 1 (from 5′ to 3′) is attached to a vinyl phosphonate moiety.

The first modified oligonucleotide of Compound 1755072 is represented by the following chemical notation: VP-TₑₛC_{fs}A_{yo}C_{yo}U_{yo}G_{ds}A_{yo}G_{yo}AₑₒA_{yo}U_{yo}A_{yo}C_{yo}U_{ds}G_{yo}U_{ds}C_{yo}C_{yo}C_{yo}U_{yo}U_{ys}AₑₛAₑ (SEQ ID NO: 29); wherein
A=an adenine nucleobase,
C=a cytosine nucleobase,
G=a guanine nucleobase,
T=a thymine nucleobase,
U=a uracil nucleobase,
d=a 2′-β-D-deoxyribosyl sugar moiety,
e=a 2′-MOE sugar moiety,
f=a 2′-fluoro sugar moiety,
y=a 2′-OMe sugar moiety,
o=a phosphodiester internucleoside linkage,
s=a phosphorothioate internucleoside linkage, and
VP=a 5′ vinyl phosphonate moiety.

### Second Oligomeric Compound of Compound No. 1755072:

The second oligomeric compound of Compound No. 1755072, which is Compound No. 1735443, has a nucleobase sequence of (from 5′ to 3′) AAGGGACAGUAUUCUCAGUGA (SEQ ID NO: 6), wherein each of nucleosides 1, 2, 20 and 21 (from 5′ to 3′) comprises a 2′-MOE modified sugar moiety, each of nucleosides 10 and 11 (from 5′ to 3′) comprises a 2′-fluoro modified sugar moiety, and each of nucleosides 3-9, and 12-19 (from 5′ to 3′) comprises a 2′-OMe modified sugar moiety, wherein each of the internucleoside linkages linking nucleosides 1 to 2, 2 to 3, 10 to 11, 19 to 20, and 20 to 21 (from 5′ to 3′) is a phosphorothioate internucleoside linkage and each of the internucleoside linkages linking nucleosides 3 to 4, 4 to 5, 5 to 6, 6 to 7, 7 to 8, 8 to 9, 9 to 10, 11 to 12, 12 to 13, 13 to 14, 14 to 15, 15 to 16, 16 to 17, 17 to 18, and 18 to 19 (from 5′ to 3′) is a phosphodiester linkage, and wherein nucleoside 1 (from 5′ to 3′) is attached is attached through the 5′-OH of the oligonucleotide to a conjugate group comprising [THA-GalNAc].

The second oligomeric compound of Compound 1755072 is represented by the following chemical notation: [THA-GalNAc]-AₑₛAₑₛG_{yo}G_{yo}G_{yo}A_{yo}C_{yo}A_{yo}G_{yo}U_{fs}A_{fo}U_{yo}U_{yo}C_{yo}U_{yo}C_{yo}A_{yo}G_{yo}U_{ys}GₑₛAₑ (SEQ ID NO: 40); wherein:

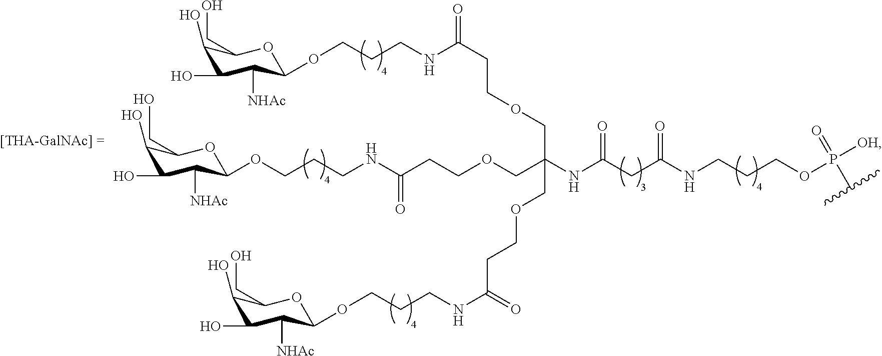

A=an adenine nucleobase,
C=a cytosine nucleobase,
G=a guanine nucleobase,
T=a thymine nucleobase,
U=a uracil nucleobase,
e=a 2′-MOE sugar moiety,
f=a 2′-fluoro sugar moiety,
y=a 2′-OMe sugar moiety,
o=a phosphodiester internucleoside linkage, and
s=a phosphorothioate internucleoside linkage.

The following chemical structure is one structural representation of Compound No. 1755072:

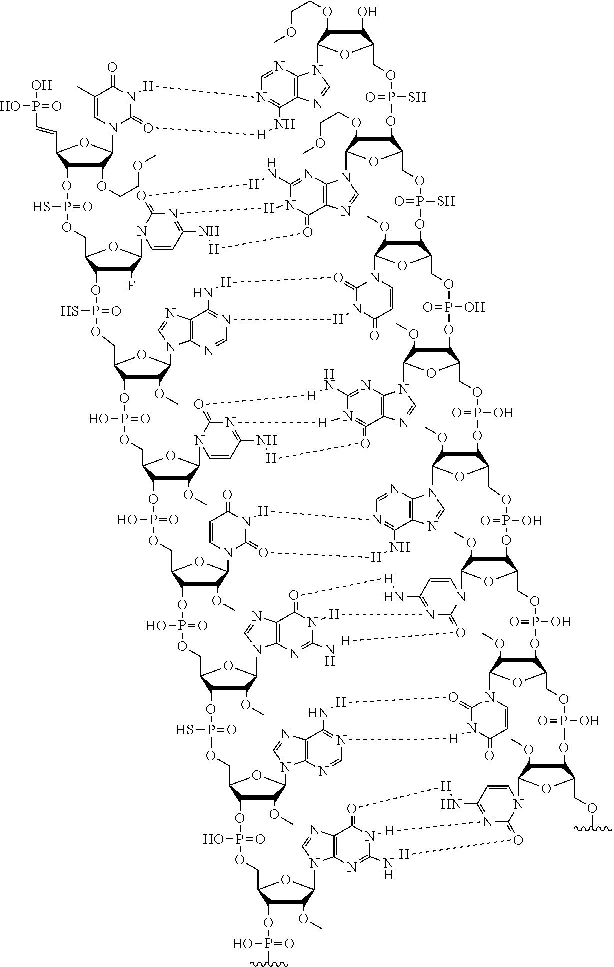

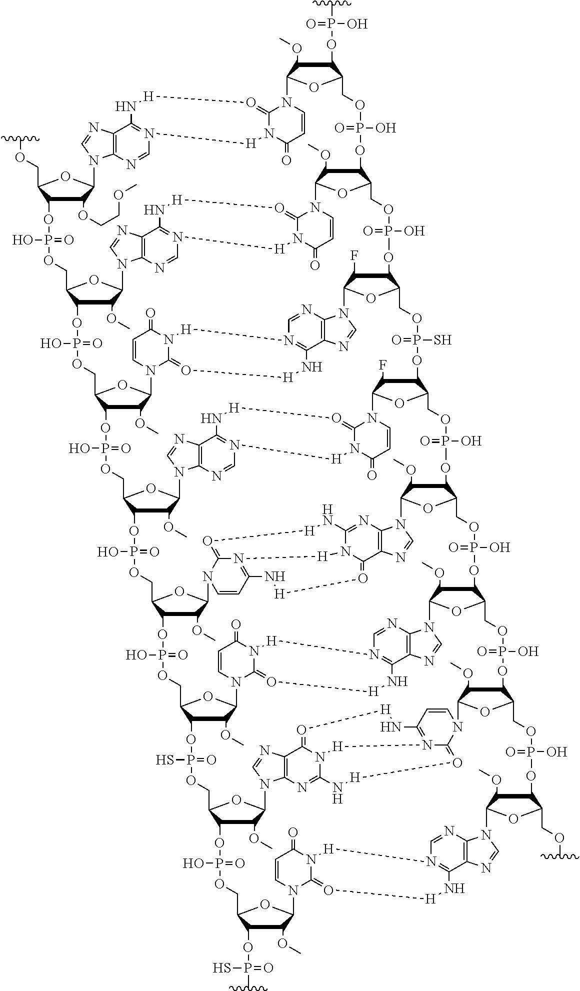

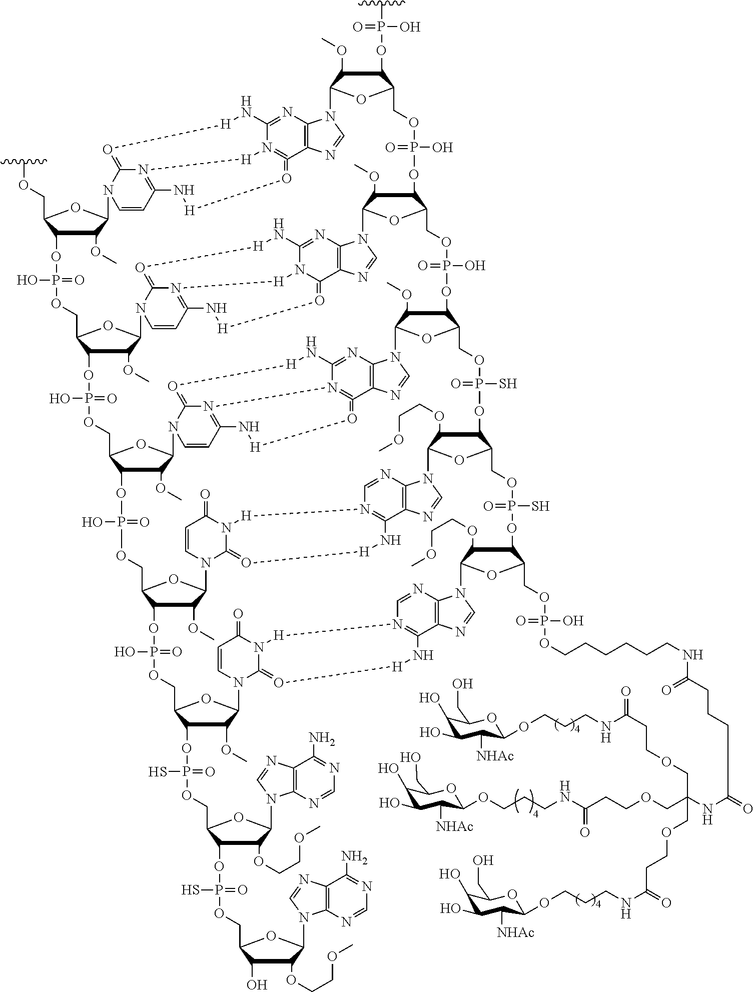

(SEQ ID NO: 29 and SEQ ID NO: 40), or an ion or salt thereof.

In certain embodiments, Compound No. 1755072 is in the form of an anion or a salt thereof, for example, a sodium salt. In certain embodiments, the oligomeric duplex is in anionic form in a solution. In certain embodiments, Compound No. 1755072 is a sodium salt or a potassium salt.

The following chemical structure represents Compound No. 1755072 in sodium solution:

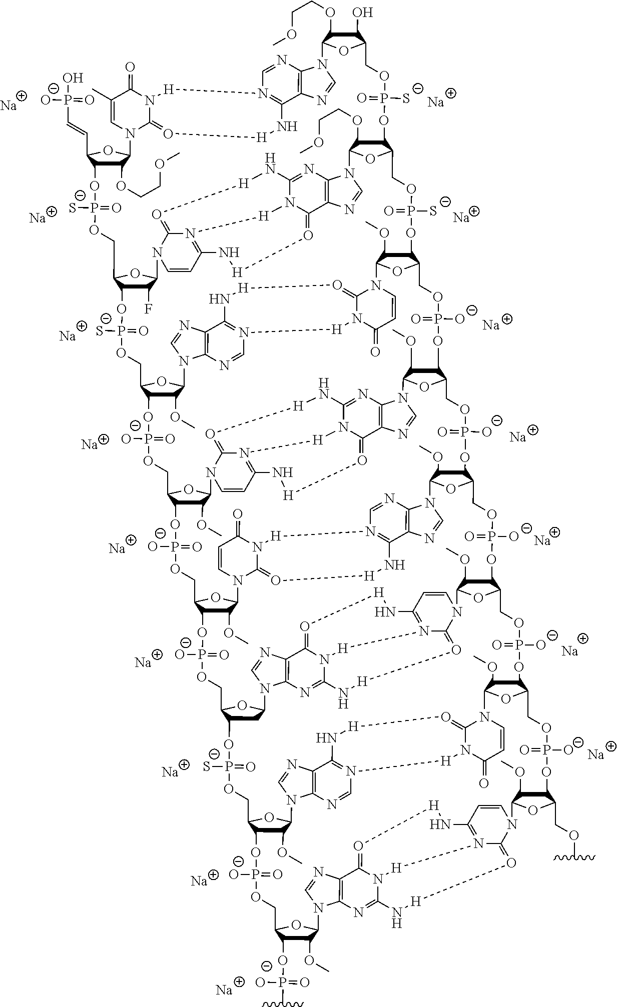

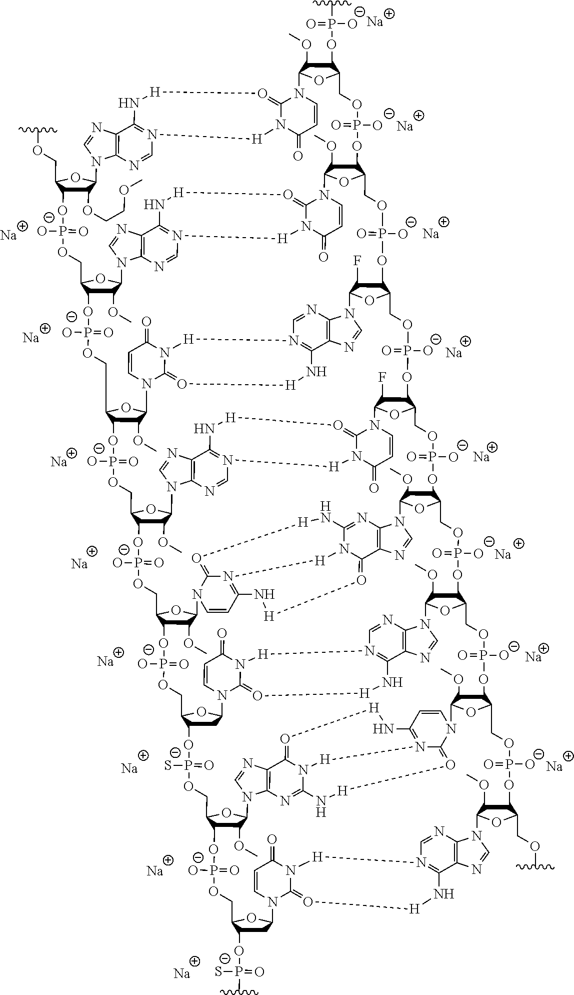

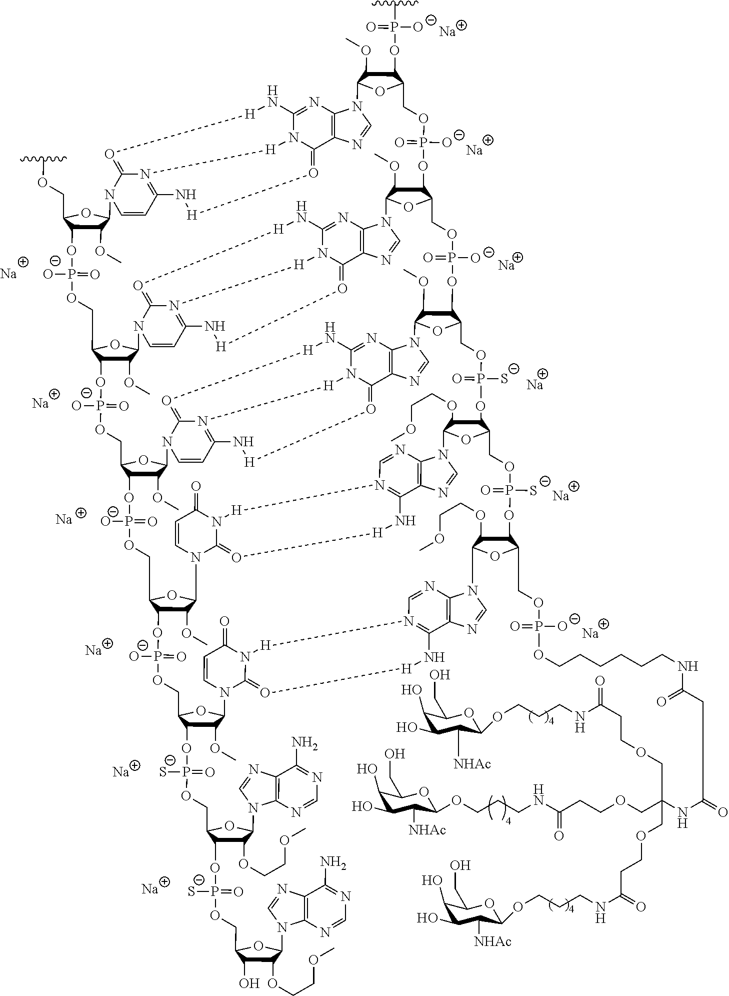

### (SEQ ID NO: 29 and SEQ ID NO: 40).

In certain embodiments, provided herein are oligomeric duplexes comprising or consisting of a first oligomeric compound containing a first modified oligonucleotide and a second oligomeric compound containing a second modified oligonucleotide as follows.

The first modified oligonucleotide is Compound No. 1753167 (SEQ ID NO: 29), as described in e. above for Compound no. 1755072.

The second oligomeric compound has a nucleobase sequence of (from 5′ to 3′) AAGGGACAGUAUUCUCAGUGA (SEQ ID NO: 6), wherein each of nucleosides 1, 2, 20 and 21 (from 5′ to 3′) comprises a 2′-MOE modified sugar moiety, each of nucleosides 10 and 11 (from 5′ to 3′) comprises a 2′-fluoro modified sugar moiety, and each of nucleosides 3-9, and 12-19 (from 5′ to 3′) comprises a 2′-OMe modified sugar moiety, wherein each of the internucleoside linkages linking nucleosides 1 to 2, 2 to 3, 10 to 11, 19 to 20, and 20 to 21 (from 5′ to 3′) is a phosphorothioate internucleoside linkage and each of the internucleoside linkages linking nucleosides 3 to 4, 4 to 5, 5 to 6, 6 to 7, 7 to 8, 8 to 9, 9 to 10, 11 to 12, 12 to 13, 13 to 14, 14 to 15, 15 to 16, 16 to 17, 17 to 18, and 18 to 19 (from 5′ to 3′) is a phosphodiester linkage.

The second oligomeric compound is represented by the following chemical notation: AₑₛAₑₛG_{yo}G_{yo}G_{yo}A_{yo}C_{yo}A_{yo}G_{yo}U_{fs}A_{fo}U_{yo}U_{yo}C_{yo}U_{yo}C_{yo}A_{yo}G_{yo}U_{ys}GₑₛAₑ (SEQ ID NO: 80); wherein:
A=an adenine nucleobase,
C=a cytosine nucleobase,
G=a guanine nucleobase,
T=a thymine nucleobase,
U=a uracil nucleobase,
e=a 2′-MOE sugar moiety,
f=a 2′-fluoro sugar moiety,
y=a 2′-OMe sugar moiety,
o=a phosphodiester internucleoside linkage, and
s=a phosphorothioate internucleoside linkage.

In certain embodiments, such an oligomeric duplex is represented by the following chemical structure:

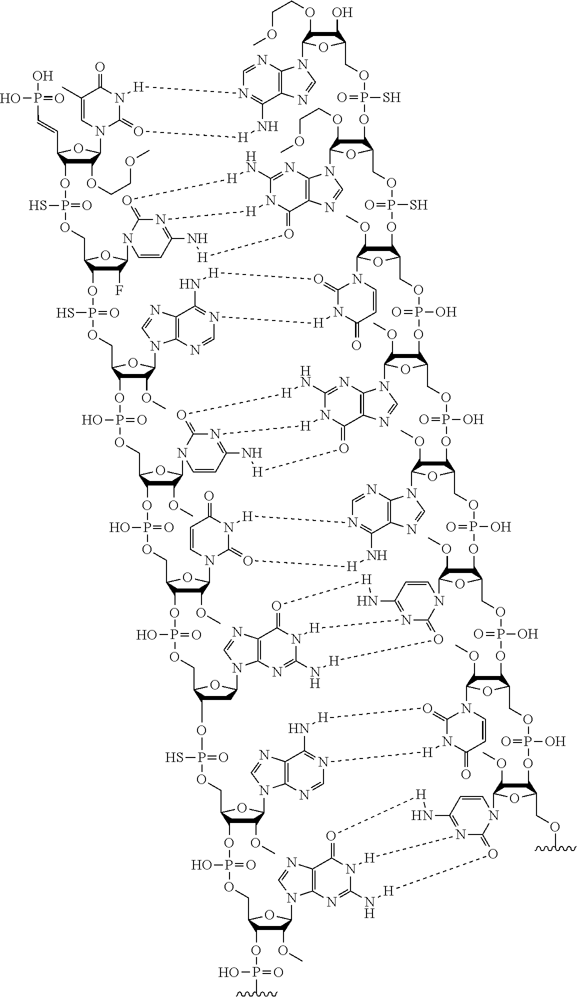

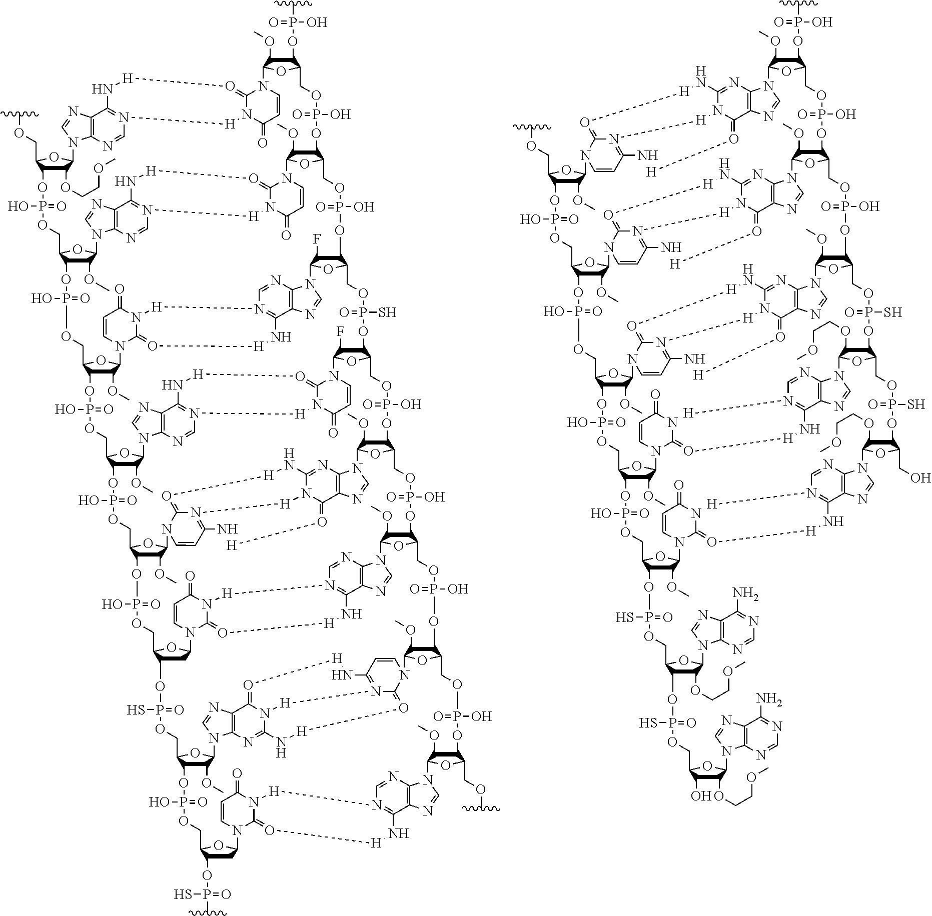

(SEQ ID NO: 29 and SEQ ID NO: 80), or an ion or salt thereof.

In certain embodiments, such an oligomeric duplex is in the form of an anion or a salt thereof, for example, a sodium salt. In certain embodiments, the oligomeric duplex is in anionic form in a solution. In certain embodiments, the oligomeric duplex is a sodium salt or a potassium salt.

The following chemical structure represents such an oligomeric duplex in sodium solution:

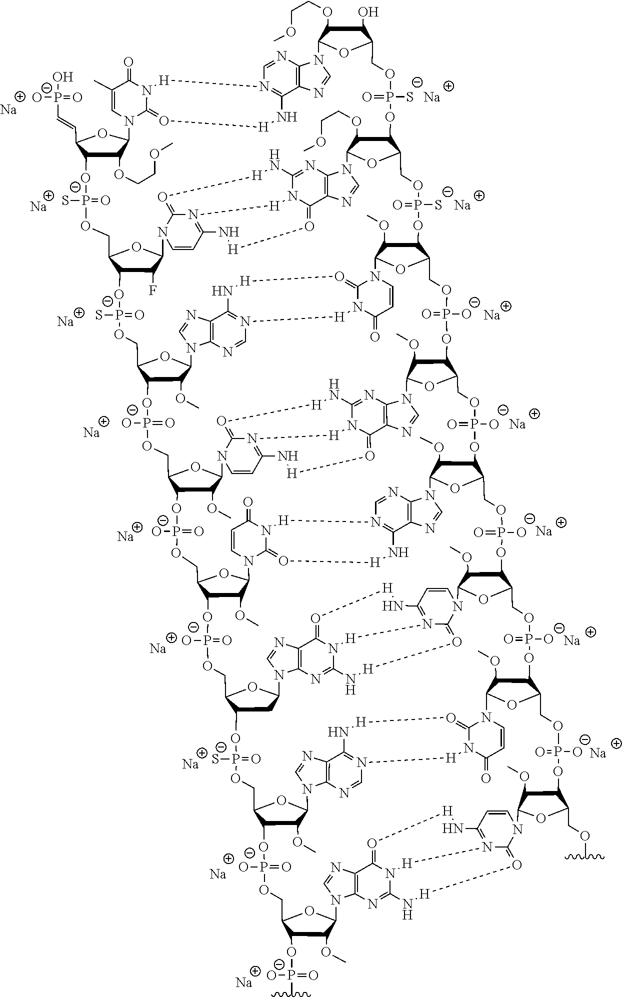

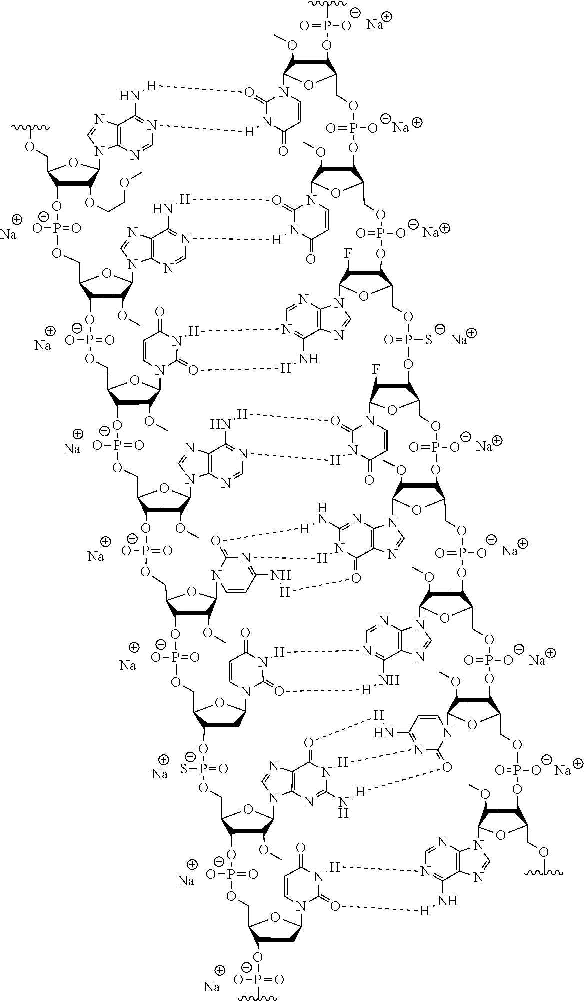

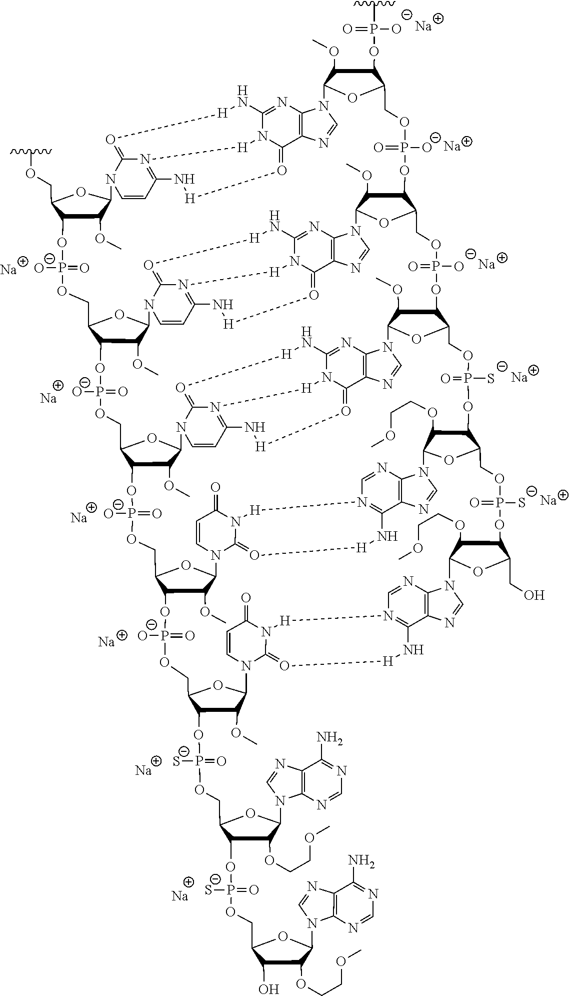

### (SEQ ID NO: 29 and SEQ ID NO: 80).

f. Compound No. 1757508

Provided herein is Compound No. 1757508, which is an oligomeric duplex that consists of a first oligomeric compound containing a first modified oligonucleotide and a second oligomeric compound containing a second modified oligonucleotide attached to a conjugate group as follows.

### First Oligomeric Compound of Compound No. 1757508:

The first modified oligonucleotide of Compound no. 1757508, which is Compound No. 1744807, has a nucleobase sequence of (from 5′ to 3′) TCACUGAGAAUACUGUCCCUUAA (SEQ ID NO: 3), wherein each of nucleosides 1, 22 and 23 (from 5′ to 3′) comprises a 2′-MOE modified sugar moiety, nucleoside 2 (from 5′ to 3′) comprises a 2′-fluoro modified sugar moiety, each of nucleosides 3-5, 7-13, 15 and 17-21 (from 5′ to 3′) comprises a 2′-OMe modified sugar moiety, and each of nucleosides 6, 14, 16 (from 5′ to 3′) is a 2′-deoxynucleoside, wherein each of the internucleoside linkages linking nucleosides 1 to 2, 2 to 3, 21 to 22, and 22 to 23 (from 5′ to 3′) is a phosphorothioate internucleoside linkage and each of the internucleoside linkages linking nucleosides 3 to 4, 4 to 5, 5 to 6, 6 to 7, 7 to 8, 8 to 9, 9 to 10, 10 to 11, 11 to 12, 12 to 13, 13 to 14, 14 to 15, 15 to 16, 16 to 17, 17 to 18, 18 to 19, 19 to 20, and 20 to 21 (from 5′ to 3′) is a phosphodiester linkage, and wherein nucleoside 1 (from 5′ to 3′) is attached to a vinyl phosphonate moiety.

The first modified oligonucleotide of Compound 1757508 is represented by the following chemical notation: VP-TₑₛC_{fs}A_{yo}C_{yo}U_{yo}G_{do}A_{yo}G_{yo}A_{yo}A_{yo}U_{yo}A_{yo}C_{yo}U_{do}G_{yo}U_{do}C_{yo}C_{yo}C_{yo}U_{yo}U_{ys}AₑₛAₑ (SEQ ID NO: 23); wherein
A=an adenine nucleobase,
C=a cytosine nucleobase,
G=a guanine nucleobase,
T=a thymine nucleobase,
U=a uracil nucleobase,
d=a 2′-β-D-deoxyribosyl sugar moiety,
e=a 2′-MOE sugar moiety,
f=a 2′-fluoro sugar moiety,
y=a 2′-OMe sugar moiety,
o=a phosphodiester internucleoside linkage,
s=a phosphorothioate internucleoside linkage, and
VP=a 5′ vinyl phosphonate moiety.

### Second Oligomeric Compound of Compound No. 1757508:

The second oligomeric compound of Compound No. 1757508, which is Compound No. 1735443, has a nucleobase sequence of (from 5′ to 3′) AAGGGACAGUAUUCUCAGUGA (SEQ ID NO: 6), wherein each of nucleosides 1, 2, 20 and 21 (from 5′ to 3′) comprises a 2′-MOE modified sugar moiety, each of nucleosides 10 and 11 (from 5′ to 3′) comprises a 2′-fluoro modified sugar moiety, and each of nucleosides 3-9, and 12-19 (from 5′ to 3′) comprises a 2′-OMe modified sugar moiety, wherein each of the internucleoside linkages linking nucleosides 1 to 2, 2 to 3, 10 to 11, 19 to 20, and 20 to 21 (from 5′ to 3′) is a phosphorothioate internucleoside linkage and each of the internucleoside linkages linking nucleosides 3 to 4, 4 to 5, 5 to 6, 6 to 7, 7 to 8, 8 to 9, 9 to 10, 11 to 12, 12 to 13, 13 to 14, 14 to 15, 15 to 16, 16 to 17, 17 to 18, and 18 to 19 (from 5′ to 3′) is a phosphodiester linkage, and wherein nucleoside 1 (from 5′ to 3′) is attached is attached through the 5′-OH of the oligonucleotide to a conjugate group comprising [THA-GalNAc].

The second oligomeric compound of Compound 1757508 is represented by the following chemical notation: [THA-GalNAc]-AₑₛAₑₛG_{yo}G_{yo}G_{yo}A_{yo}C_{yo}A_{yo}G_{yo}U_{fs}A_{fo}U_{yo}U_{yo}C_{yo}U_{yo}C_{yo}A_{yo}G_{yo}U_{ys}GₑₛAₑ (SEQ ID NO: 40); wherein:

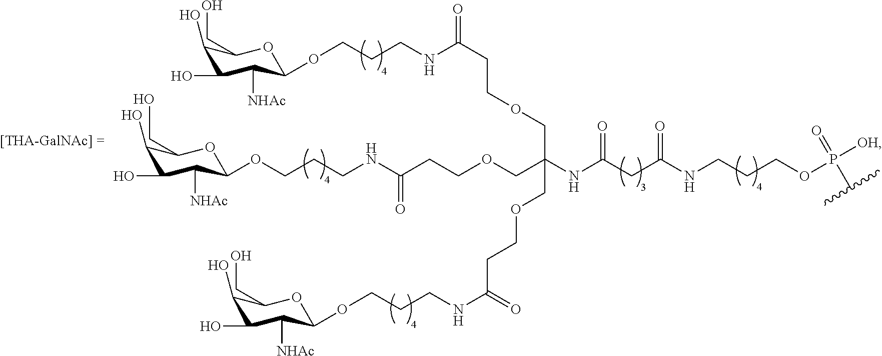

A=an adenine nucleobase,
C=a cytosine nucleobase,
G=a guanine nucleobase,
T=a thymine nucleobase,
U=a uracil nucleobase,
e=a 2′-MOE sugar moiety,
f=a 2′-fluoro sugar moiety,
y=a 2′-OMe sugar moiety,
o=a phosphodiester internucleoside linkage, and
s=a phosphorothioate internucleoside linkage.

The following chemical structure is one structural representation of Compound No. 1757508:

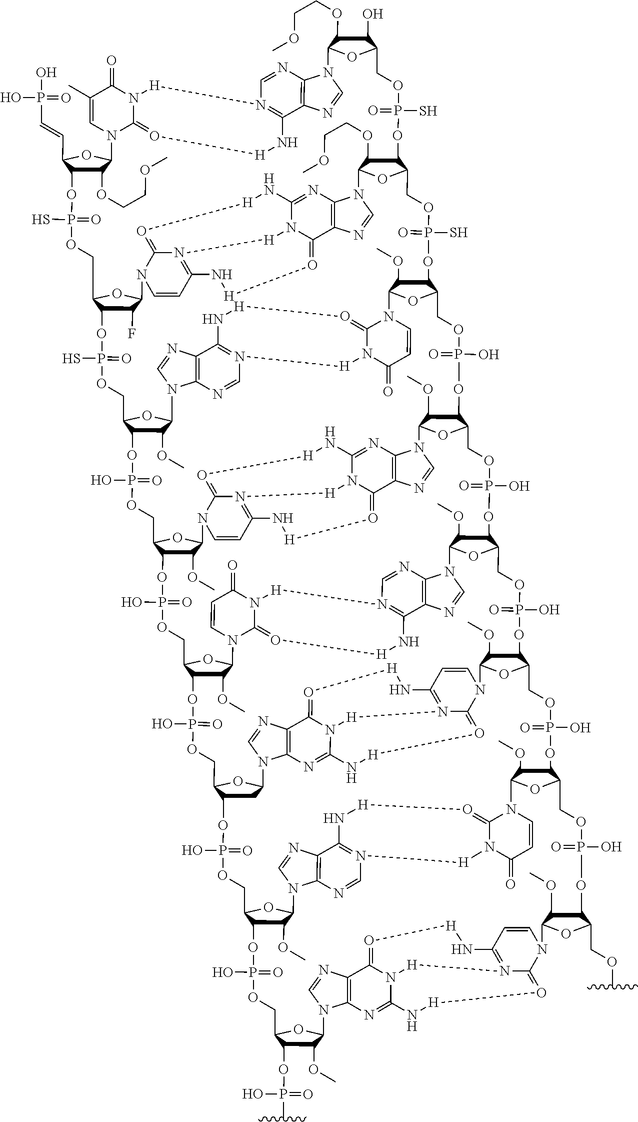

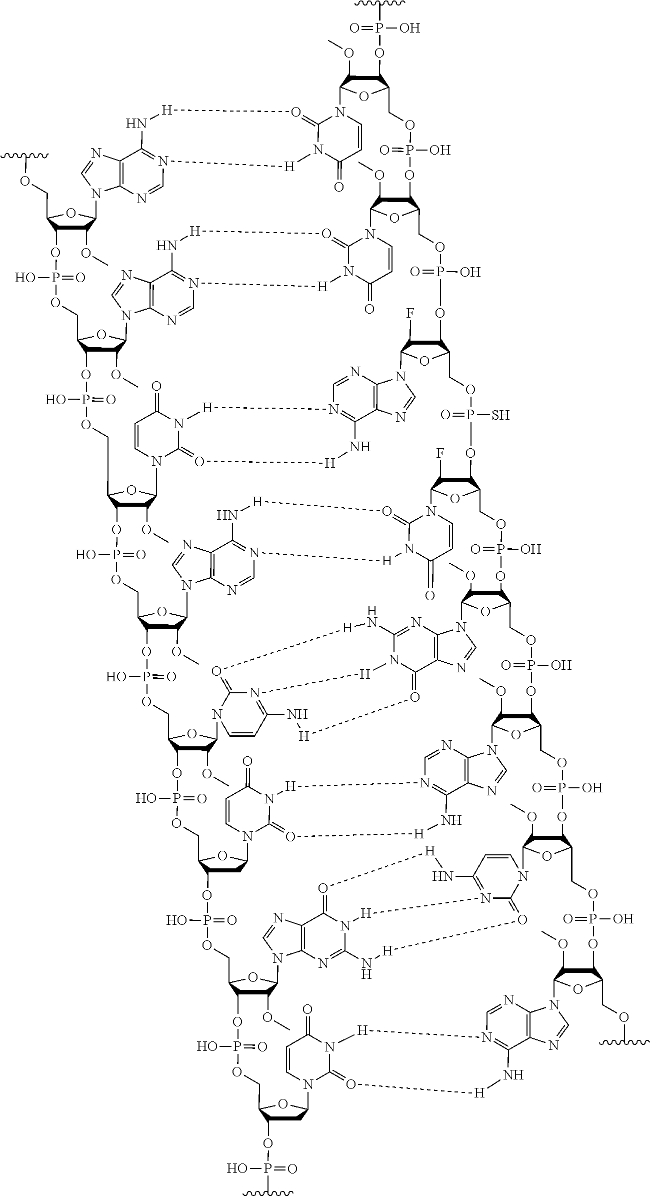

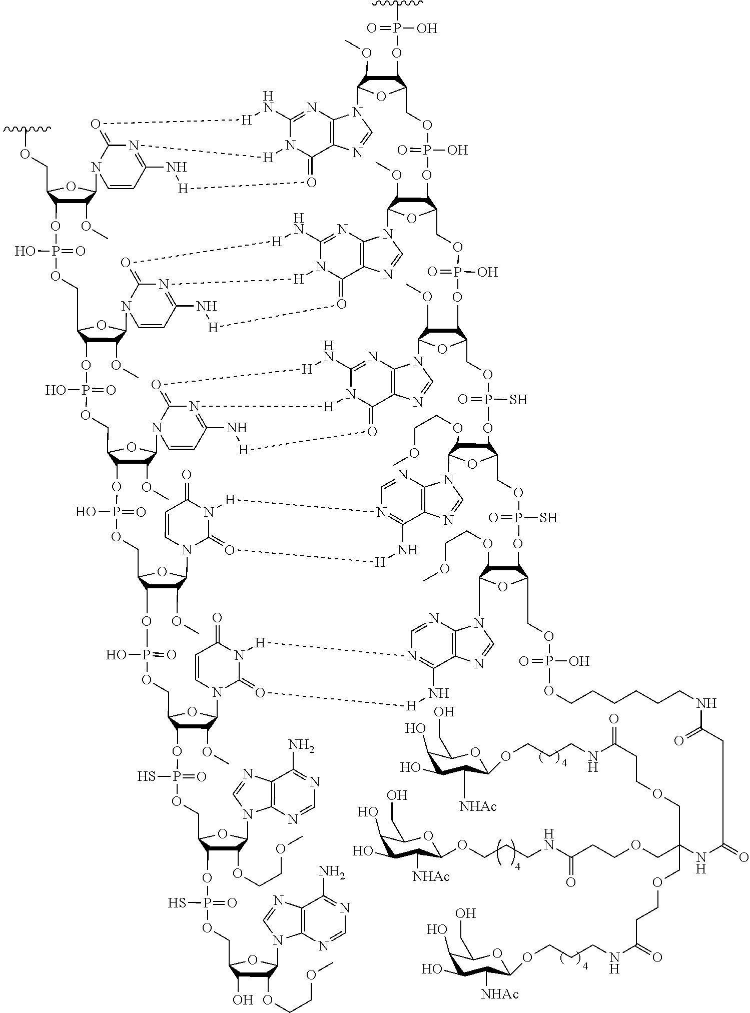

(SEQ ID NO: 23 and SEQ ID NO: 40), or an ion or salt thereof.

In certain embodiments, Compound No. 1757508 is in the form of an anion or a salt thereof, for example, the oligomeric duplex may be in the form of a sodium salt. In certain embodiments, the oligomeric duplex is in anionic form in a solution. In certain embodiments, Compound No. 1757508 is a sodium salt or a potassium salt.

The following chemical structure represents Compound No. 1757508 in sodium solution:

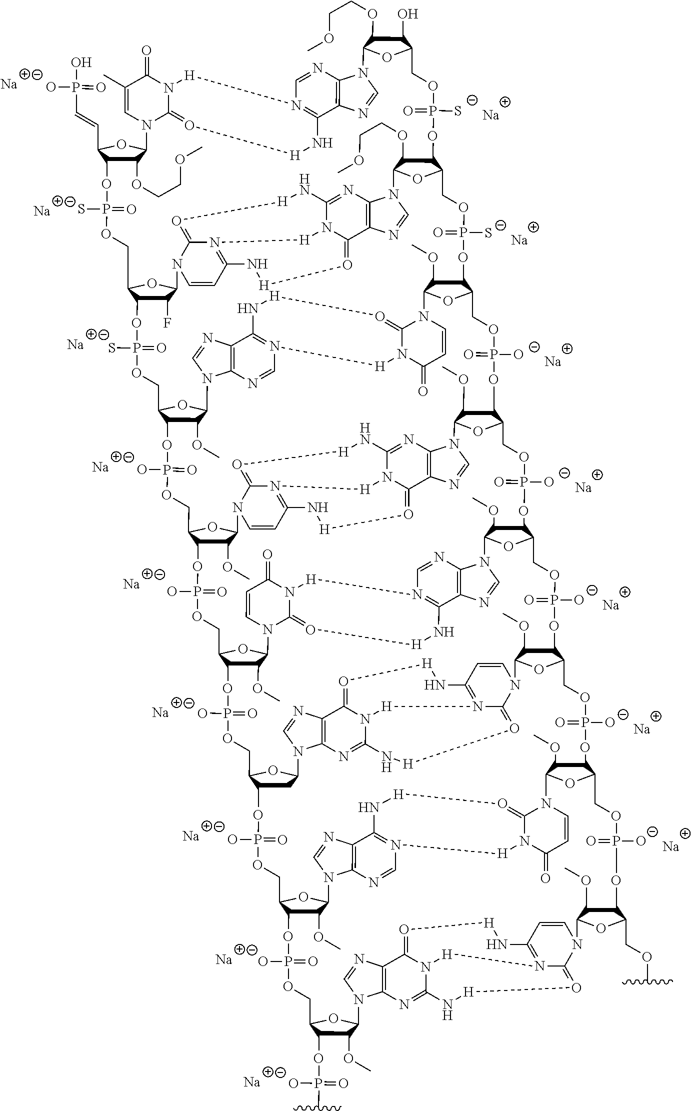

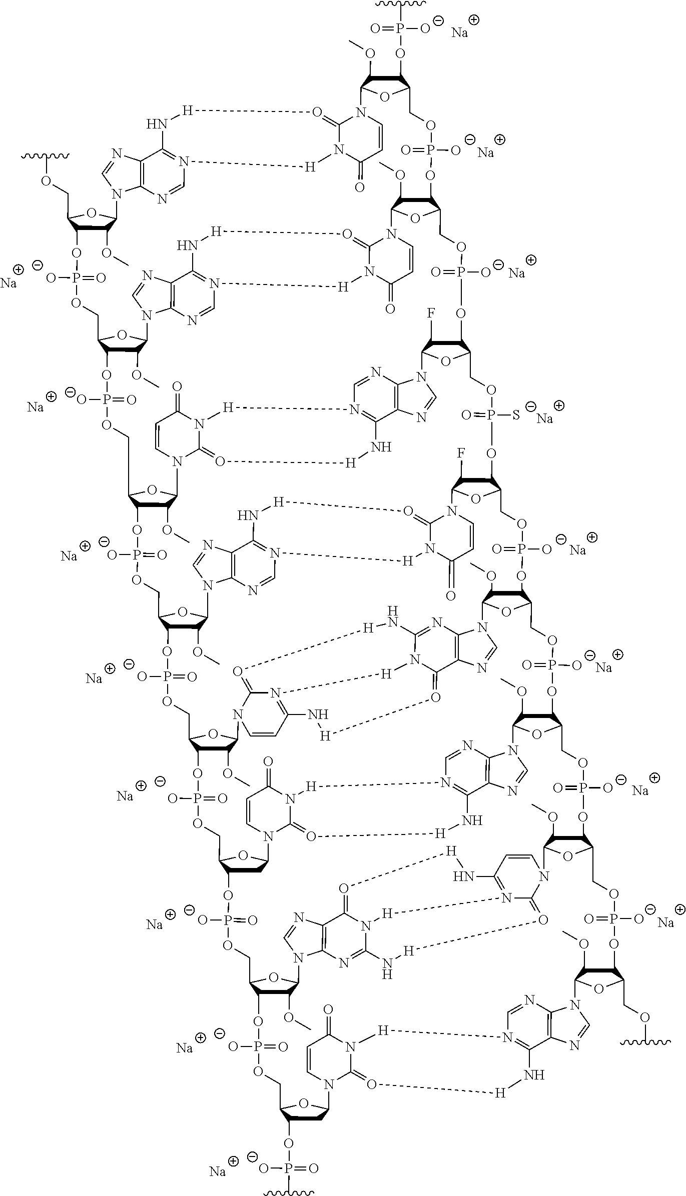

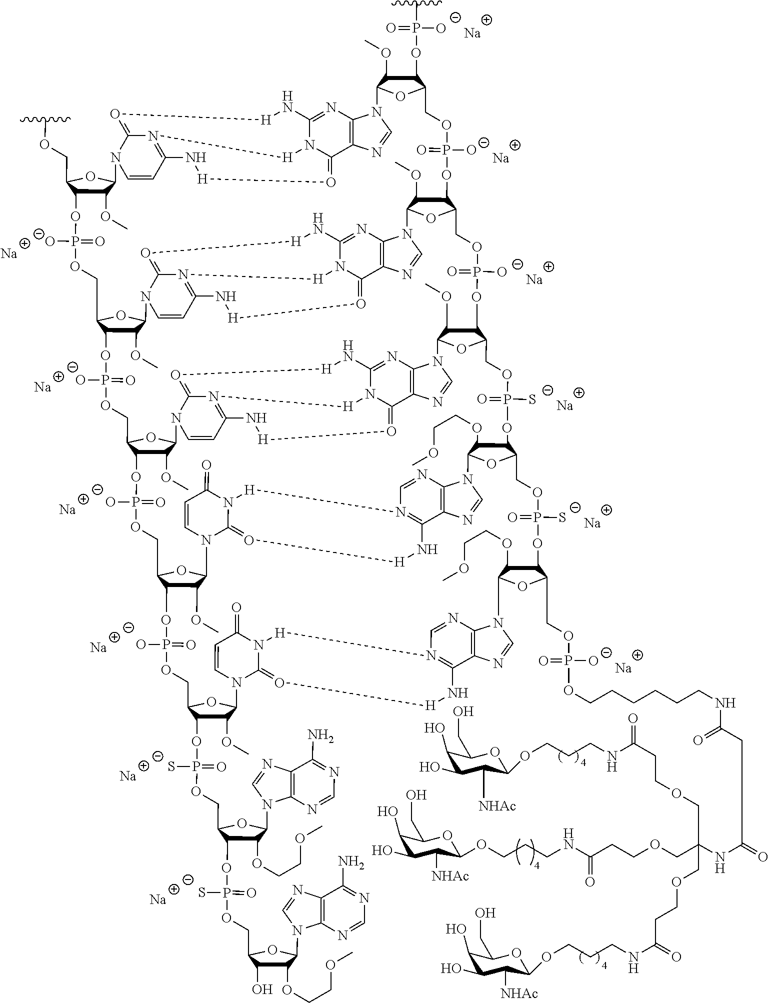

### (SEQ ID NO: 23 and SEQ ID NO: 40).

In certain embodiments, provided herein are oligomeric duplexes comprising or consisting of a first oligomeric compound containing a first modified oligonucleotide and a second oligomeric compound containing a second modified oligonucleotide as follows.

The first modified oligonucleotide is Compound No. 1744807, as described in f. above for Compound no. 1757508.

The second oligomeric compound has a nucleobase sequence of (from 5′ to 3′) AAGGGACAGUAUUCUCAGUGA (SEQ ID NO: 6), wherein each of nucleosides 1, 2, 20 and 21 (from 5′ to 3′) comprises a 2′-MOE modified sugar moiety, each of nucleosides 10 and 11 (from 5′ to 3′) comprises a 2′-fluoro modified sugar moiety, and each of nucleosides 3-9, and 12-19 (from 5′ to 3′) comprises a 2′-OMe modified sugar moiety, wherein each of the internucleoside linkages linking nucleosides 1 to 2, 2 to 3, 10 to 11, 19 to 20, and 20 to 21 (from 5′ to 3′) is a phosphorothioate internucleoside linkage and each of the internucleoside linkages linking nucleosides 3 to 4, 4 to 5, 5 to 6, 6 to 7, 7 to 8, 8 to 9, 9 to 10, 11 to 12, 12 to 13, 13 to 14, 14 to 15, 15 to 16, 16 to 17, 17 to 18, and 18 to 19 (from 5′ to 3′) is a phosphodiester linkage.

The second oligomeric compound is represented by the following chemical notation: AₑₛAₑₛG_{yo}G_{yo}G_{yo}A_{yo}C_{yo}A_{yo}G_{yo}U_{fs}A_{fo}U_{yo}U_{yo}C_{yo}U_{yo}C_{yo}A_{yo}G_{yo}U_{ys}GₑₛAₑ (SEQ ID NO: 80); wherein:
A=an adenine nucleobase,
C=a cytosine nucleobase,
G=a guanine nucleobase,
T=a thymine nucleobase,
U=a uracil nucleobase,
e=a 2′-MOE sugar moiety,
f=a 2′-fluoro sugar moiety,
y=a 2′-OMe sugar moiety,
o=a phosphodiester internucleoside linkage, and
s=a phosphorothioate internucleoside linkage.

In certain embodiments, such an oligomeric duplex is represented by the following chemical structure:

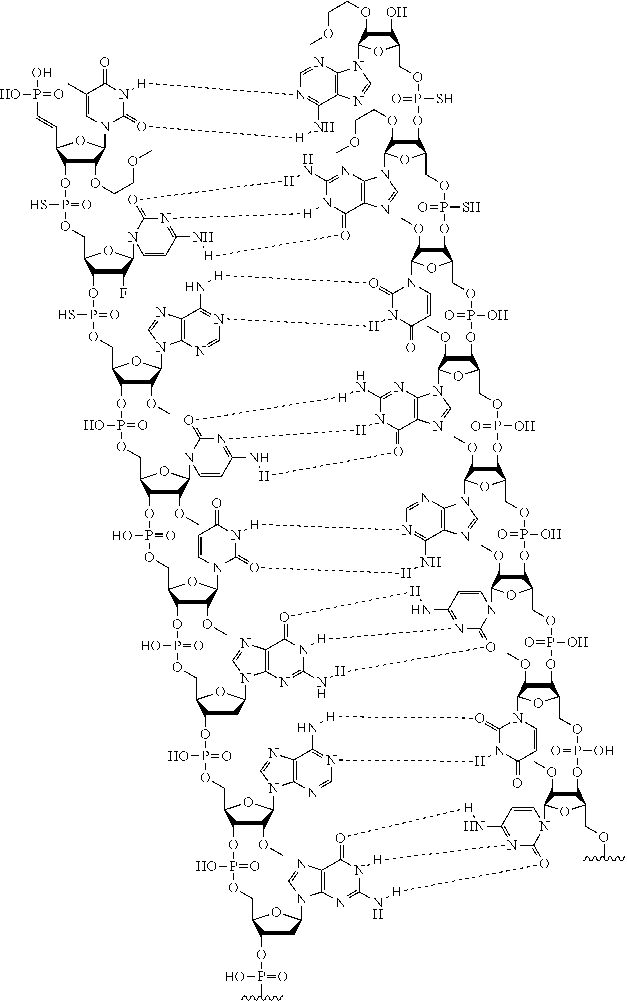

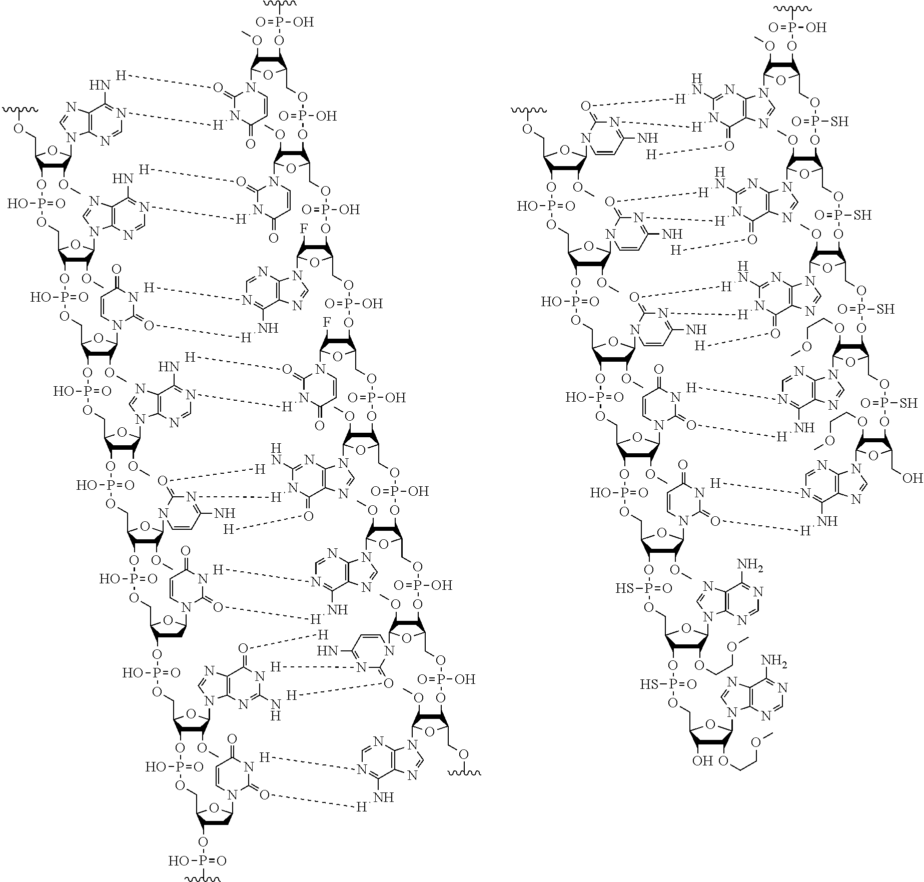

(SEQ ID NO: 23 and SEQ ID NO: 80), or an ion or salt thereof.

In certain embodiments, such an oligomeric duplex is in the form of an anion or a salt thereof, for example, a sodium salt. In certain embodiments, the oligomeric duplex is in anionic form in a solution. In certain embodiments, the oligomeric duplex is a sodium salt or a potassium salt.

The following chemical structure represents such an oligomeric duplex in sodium solution:

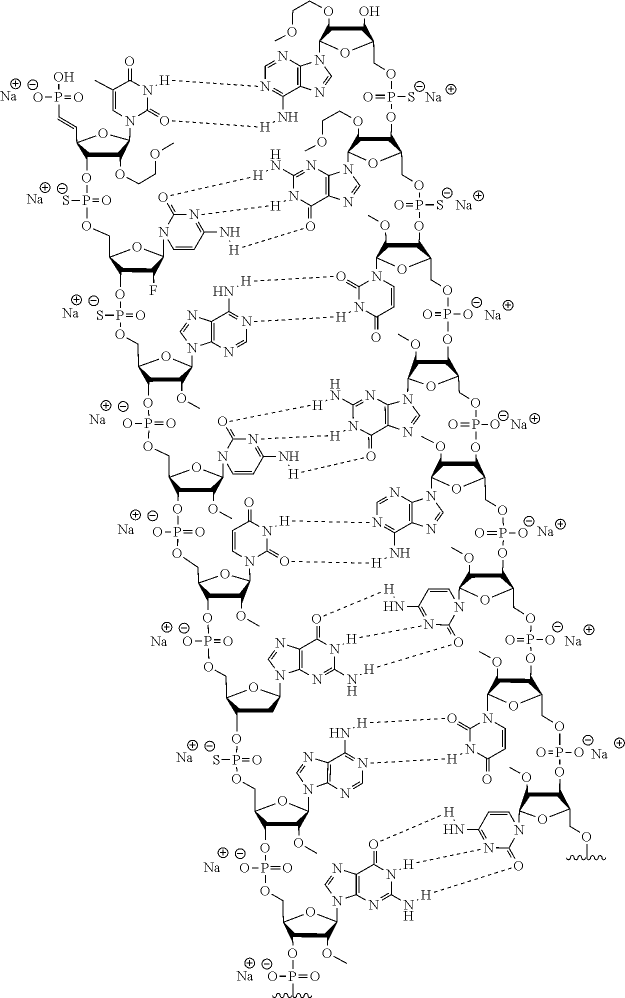

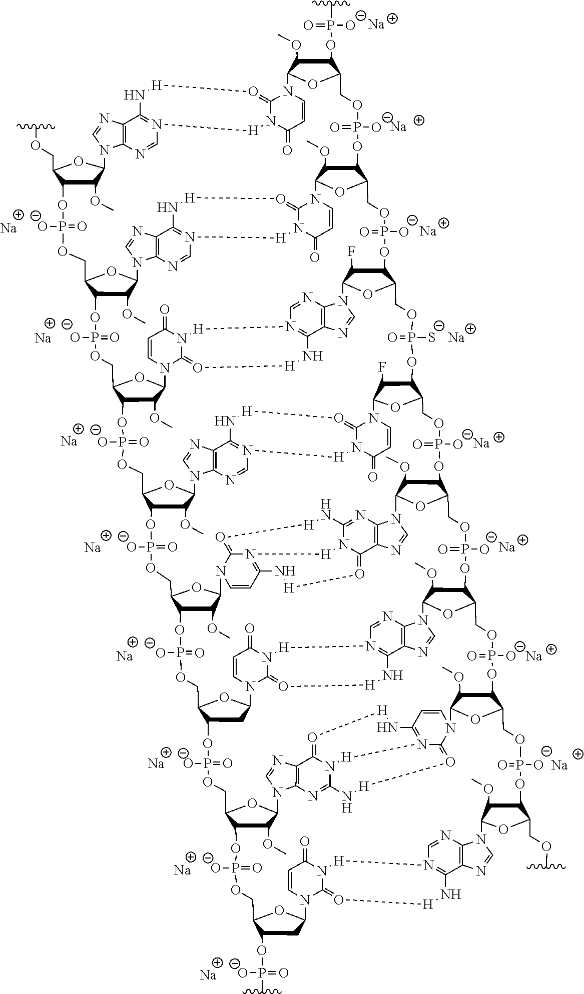

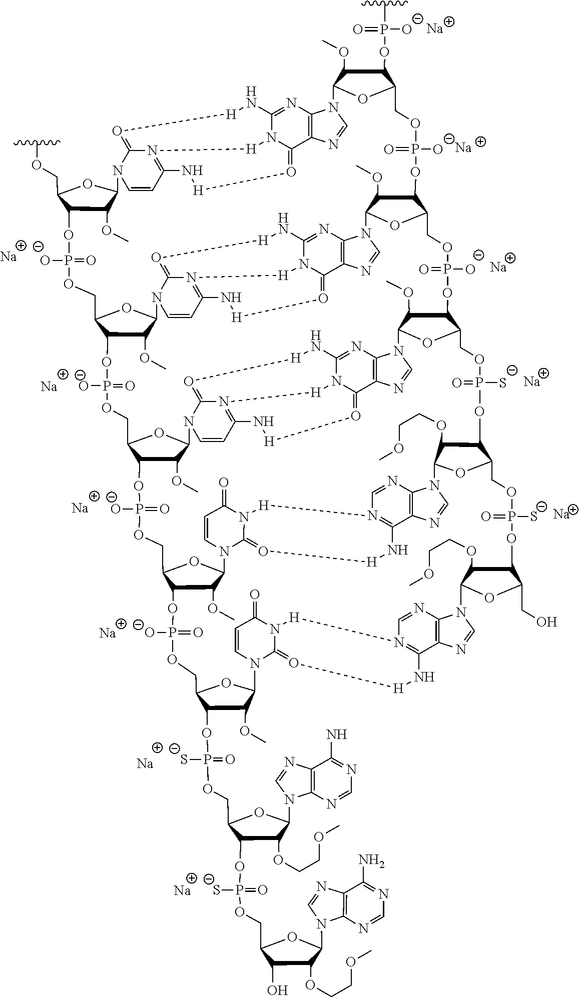

### (SEQ ID NO: 29 and SEQ ID NO: 80).

g. Compound No. 1758193

Provided herein is Compound No. 1758193, which is an oligomeric duplex that consists of a first oligomeric compound containing a first modified oligonucleotide and a second oligomeric compound containing a second modified oligonucleotide attached to a conjugate group as follows.

### First Oligomeric Compound of Compound No. 1758193:

The first modified oligonucleotide of Compound no. 1758193, which is Compound No. 1757481, has a nucleobase sequence of (from 5′ to 3′) TCACUGAGAAUACTGTCCCUUAA (SEQ ID NO: 4), wherein each of nucleosides 1, 9, 22 and 23 (from 5′ to 3′) comprises a 2′-MOE modified sugar moiety, nucleoside 2 (from 5′ to 3′) comprises a 2′-fluoro modified sugar moiety, each of nucleosides 3-5, 7, 8, 10-13, 15 and 17-21 (from 5′ to 3′) comprises a 2′-OMe modified sugar moiety, and each of nucleosides 6, 14 and 16 (from 5′ to 3′) is a 2′-deoxynucleoside, wherein each of the internucleoside linkages linking nucleosides 1 to 2, 2 to 3, 6 to 7, 14 to 15, 16 to 17, 21 to 22, and 22 to 23 (from 5′ to 3′) is a phosphorothioate internucleoside linkage and each of the internucleoside linkages linking nucleosides 3 to 4, 4 to 5, 5 to 6, 7 to 8, 8 to 9, 9 to 10, 10 to 11, 11 to 12, 12 to 13, 13 to 14, 15 to 16, 17 to 18, 18 to 19, 19 to 20, and 20 to 21 (from 5′ to 3′) is a phosphodiester linkage, and wherein nucleoside 1 (from 5′ to 3′) is attached to a vinyl phosphonate moiety.

The first oligomeric compound of Compound 1758193 is represented by the following chemical notation: VP-TₑₛC_{fs}A_{yo}C_{yo}U_{yo}G_{ds}A_{yo}G_{yo}AₑₒA_{yo}U_{yo}A_{yo}C_{yo}T_{ds}G_{yo}T_{ds}C_{yo}C_{yo}C_{yo}U_{yo}U_{ys}AₑₛAₑ (SEQ ID NO: 31); wherein
A=an adenine nucleobase,
C=a cytosine nucleobase,
G=a guanine nucleobase,
T=a thymine nucleobase,
U=a uracil nucleobase,
d=a 2′-β-D-deoxyribosyl sugar moiety,
e=a 2′-MOE sugar moiety,
f=a 2′-fluoro sugar moiety,
y=a 2′-OMe sugar moiety,
o=a phosphodiester internucleoside linkage,
s=a phosphorothioate internucleoside linkage, and
VP=a 5′ vinyl phosphonate moiety.

### Second Oligomeric Compound of Compound No. 1758193:

The second oligomeric compound of Compound No. 1758193, which is Compound No. 1718715, has a nucleobase sequence of (from 5′ to 3′) AAGGGACAGUAUUCUCAGUGA (SEQ ID NO: 6), wherein each of nucleosides 1, 2, 20 and 21 (from 5′ to 3′) comprises a 2′-MOE modified sugar moiety, each of nucleosides 10 and 11 (from 5′ to 3′) comprises a 2′-fluoro modified sugar moiety, and each of nucleosides 3-9, and 12-19 (from 5′ to 3′) comprises a 2′-OMe modified sugar moiety, wherein each of the internucleoside linkages linking nucleosides 1 to 2, 2 to 3, 19 to 20, and 20 to 21 (from 5′ to 3′) is a phosphorothioate internucleoside linkage and each of the internucleoside linkages linking nucleosides 3 to 4, 4 to 5, 5 to 6, 6 to 7, 7 to 8, 8 to 9, 9 to 10, 10 to 11, 11 to 12, 12 to 13, 13 to 14, 14 to 15, 15 to 16, 16 to 17, 17 to 18, and 18 to 19 (from 5′ to 3′) is a phosphodiester linkage, and wherein nucleoside 1 (from 5′ to 3′) is attached is attached through the 5′-OH of the oligonucleotide to a conjugate group comprising [THA-GalNAc].

The second oligomeric compound of Compound 1758193 is represented by the following chemical notation: [THA-GalNAc]-AₑₛAₑₛG_{yo}G_{yo}G_{yo}A_{yo}C_{yo}A_{yo}G_{yo}U_{fo}A_{fo}U_{yo}U_{yo}C_{yo}U_{yo}C_{yo}A_{yo}G_{yo}U_{ys}GₑₛAₑ (SEQ ID NO: 39); wherein:

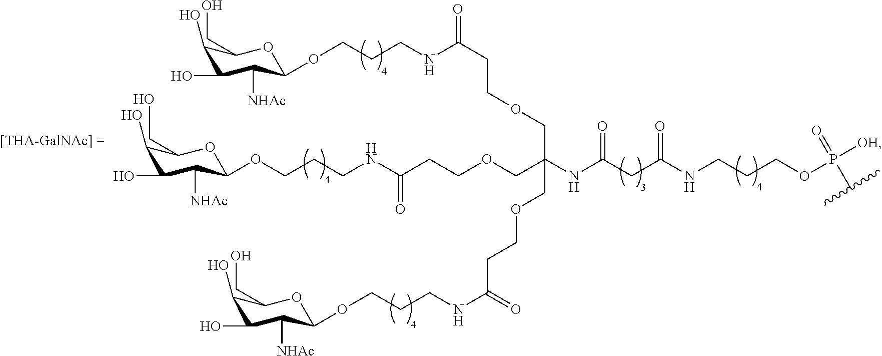

A=an adenine nucleobase,
C=a cytosine nucleobase,
G=a guanine nucleobase,
T=a thymine nucleobase,
U=a uracil nucleobase,
e=a 2′-MOE sugar moiety,
f=a 2′-fluoro sugar moiety,
y=a 2′-OMe sugar moiety,
o=a phosphodiester internucleoside linkage, and
s=a phosphorothioate internucleoside linkage.

The following chemical structure is one structural representation of Compound No. 1758193:

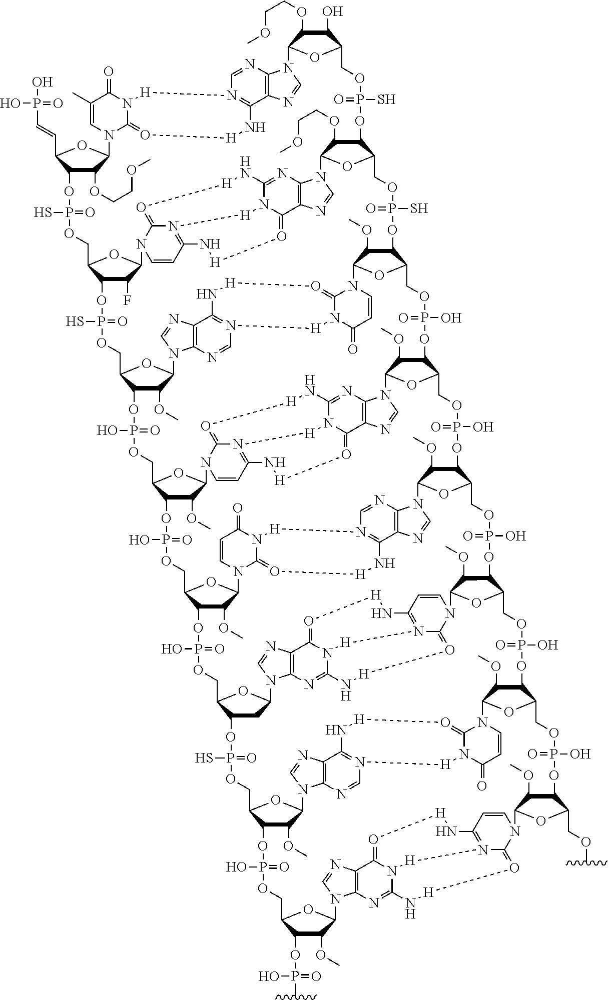

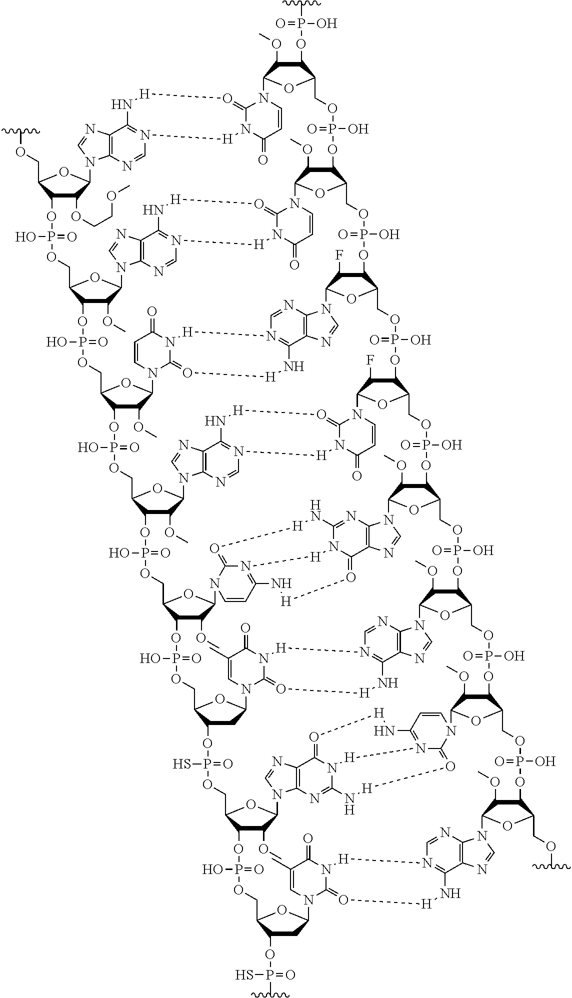

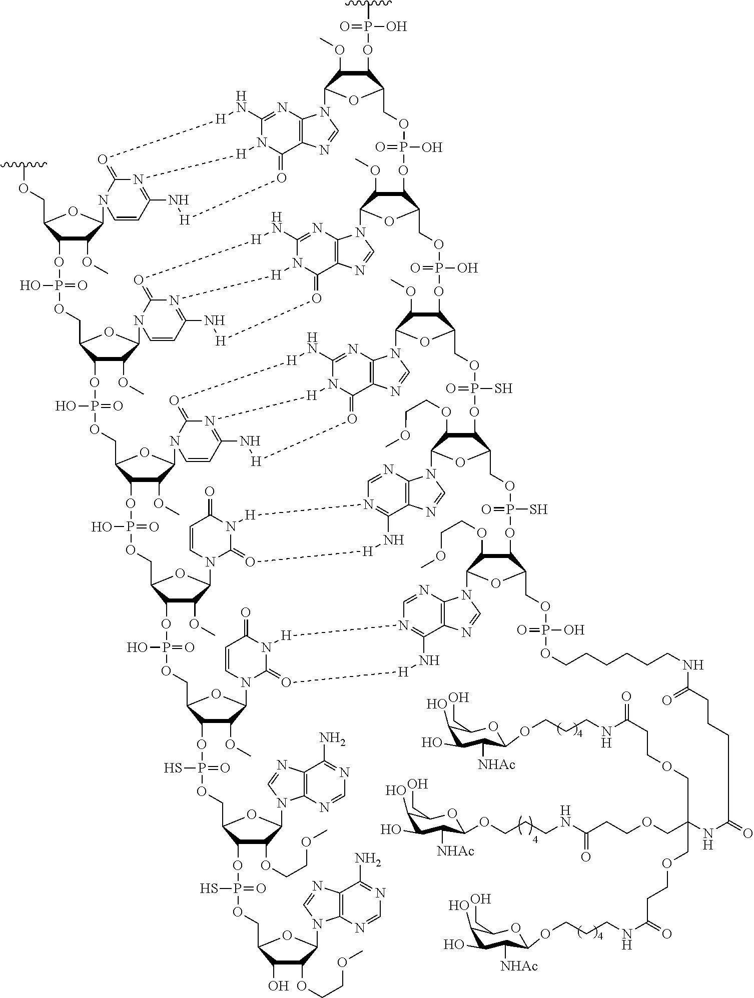

(SEQ ID NO: 31 and SEQ ID NO: 39), or an ion or salt thereof.

In certain embodiments, Compound No. 1758193 is in the form of an anion or a salt thereof, for example, the oligomeric duplex may be in the form of a sodium salt. In certain embodiments, the oligomeric duplex is in anionic form in a solution. In certain embodiments, Compound No. 1758193 is a sodium salt or a potassium salt.

The following chemical structure represents Compound No. 1758193 in sodium solution:

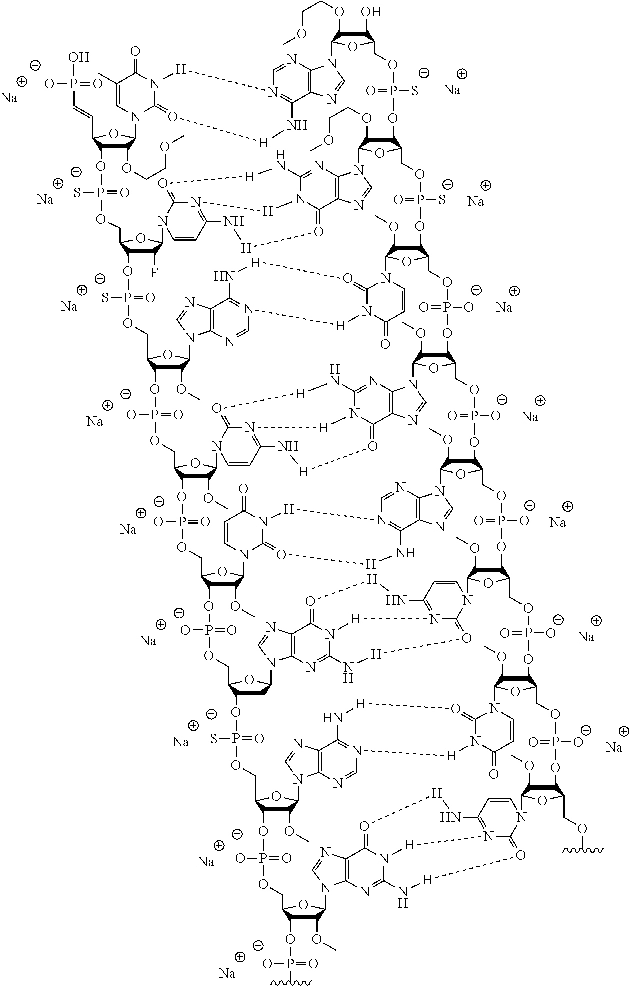

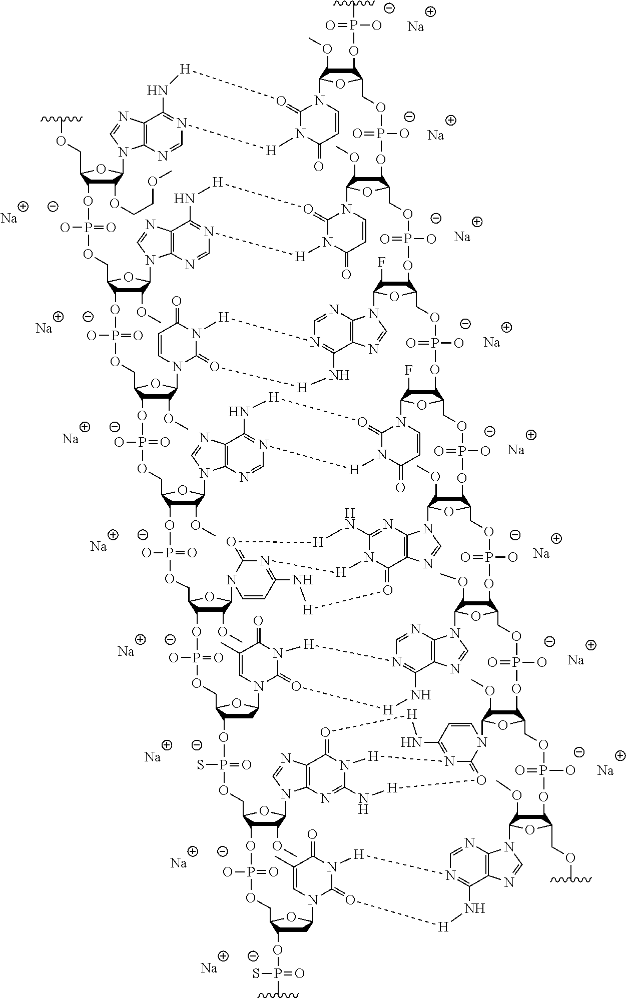

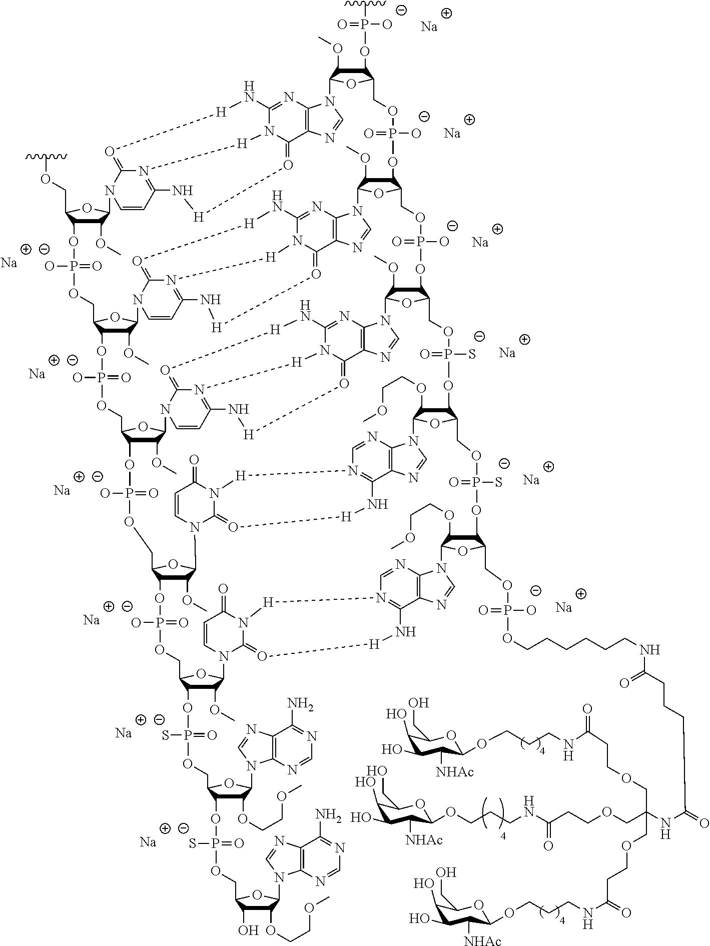

### (SEQ ID NO: 31 and SEQ ID NO: 39).

In certain embodiments, provided herein are oligomeric duplexes comprising or consisting of a first oligomeric compound containing a first modified oligonucleotide and a second oligomeric compound containing a second modified oligonucleotide as follows.

The first modified oligonucleotide is Compound No. 1757481 (SEQ ID NO: 31), as described in g. above for Compound no. 1758193.

The second oligomeric compound has a nucleobase sequence of (from 5′ to 3′) AAGGGACAGUAUUCUCAGUGA (SEQ ID NO: 6), wherein each of nucleosides 1, 2, 20 and 21 (from 5′ to 3′) comprises a 2′-MOE modified sugar moiety, each of nucleosides 10 and 11 (from 5′ to 3′) comprises a 2′-fluoro modified sugar moiety, and each of nucleosides 3-9, and 12-19 (from 5′ to 3′) comprises a 2′-OMe modified sugar moiety, wherein each of the internucleoside linkages linking nucleosides 1 to 2, 2 to 3, 19 to 20, and 20 to 21 (from 5′ to 3′) is a phosphorothioate internucleoside linkage and each of the internucleoside linkages linking nucleosides 3 to 4, 4 to 5, 5 to 6, 6 to 7, 7 to 8, 8 to 9, 9 to 10, 10 to 11, 11 to 12, 12 to 13, 13 to 14, 14 to 15, 15 to 16, 16 to 17, 17 to 18, and 18 to 19 (from 5′ to 3′) is a phosphodiester linkage.

The second oligomeric compound is represented by the following chemical notation: AₑₛAₑₛG_{yo}G_{yo}G_{yo}A_{yo}C_{yo}A_{yo}G_{yo}U_{fo}A_{fo}U_{yo}U_{yo}C_{yo}U_{yo}C_{yo}A_{yo}G_{yo}U_{ys}GₑₛAₑ (SEQ ID NO: 79); wherein:
A=an adenine nucleobase,
C=a cytosine nucleobase,
G=a guanine nucleobase,
T=a thymine nucleobase,
U=a uracil nucleobase,
e=a 2′-MOE sugar moiety,
f=a 2′-fluoro sugar moiety,
y=a 2′-OMe sugar moiety,
o=a phosphodiester internucleoside linkage, and
s=a phosphorothioate internucleoside linkage.

In certain embodiments, such an oligomeric duplex is represented by the following chemical structure:

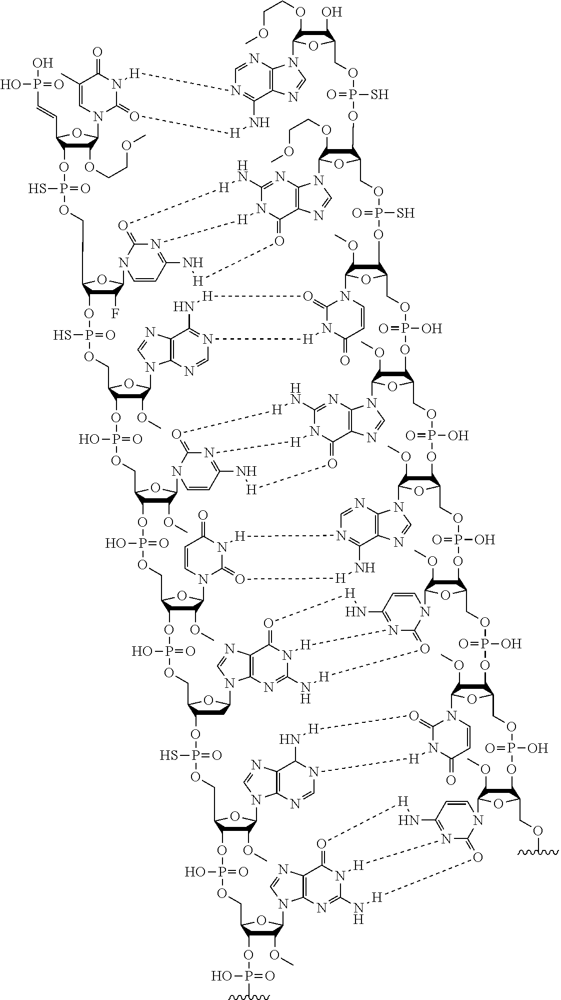

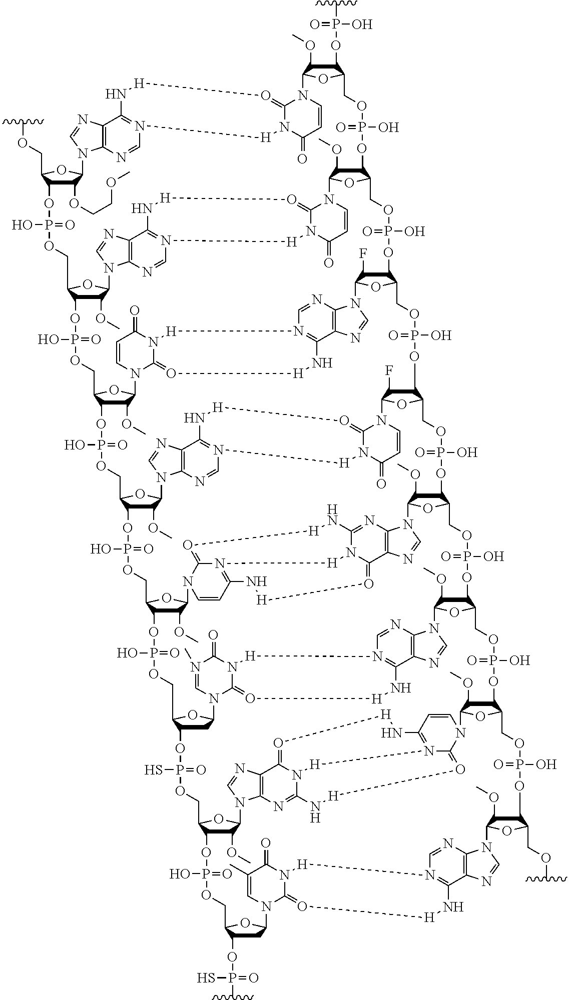

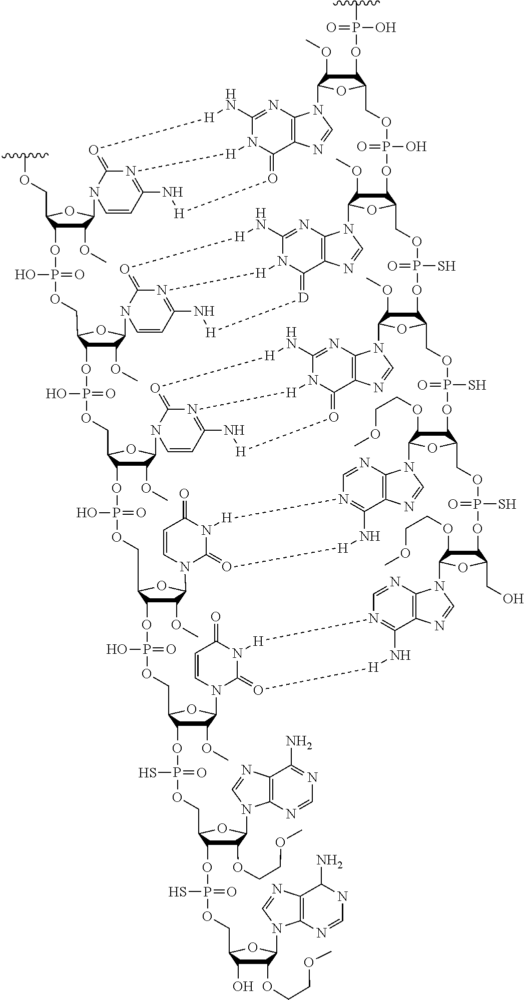

(SEQ ID NO: 31 and SEQ ID NO: 79), or an ion or salt thereof.

In certain embodiments, such an oligomeric duplex is in the form of an anion or a salt thereof, for example, a sodium salt. In certain embodiments, the oligomeric duplex is in anionic form in a solution. In certain embodiments, the oligomeric duplex is a sodium salt or a potassium salt.

The following chemical structure represents such an oligomeric duplex in sodium solution:

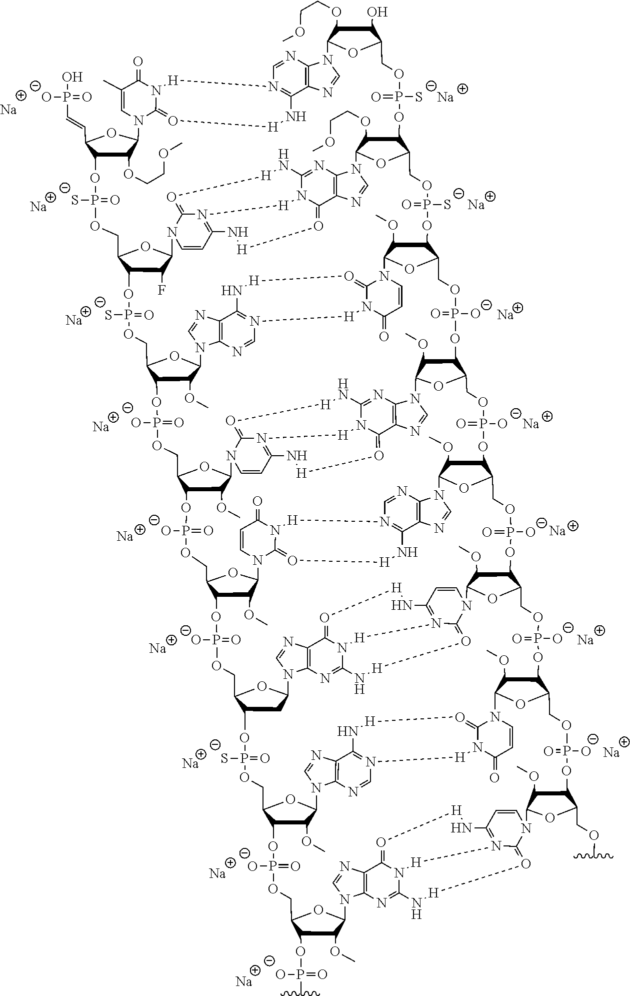

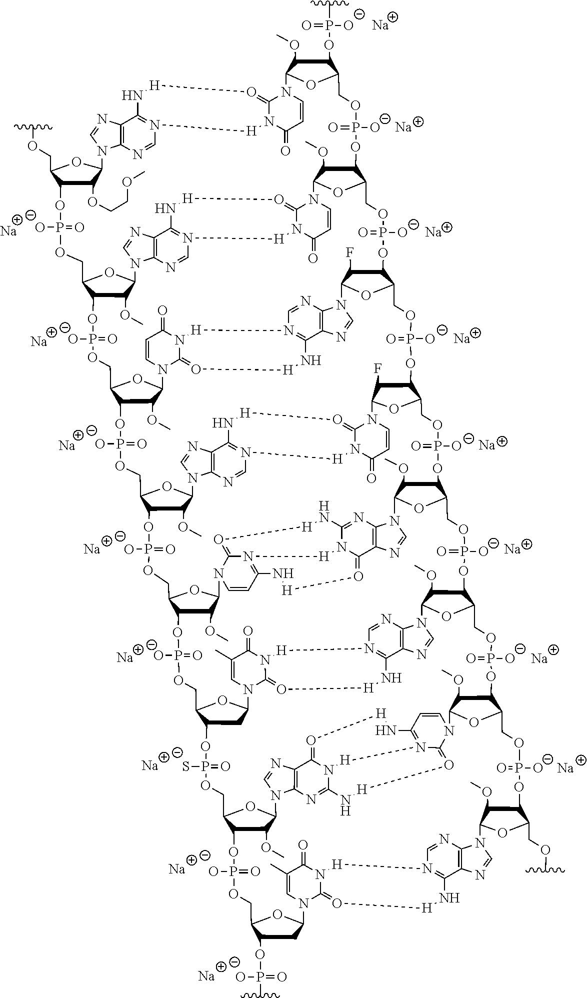

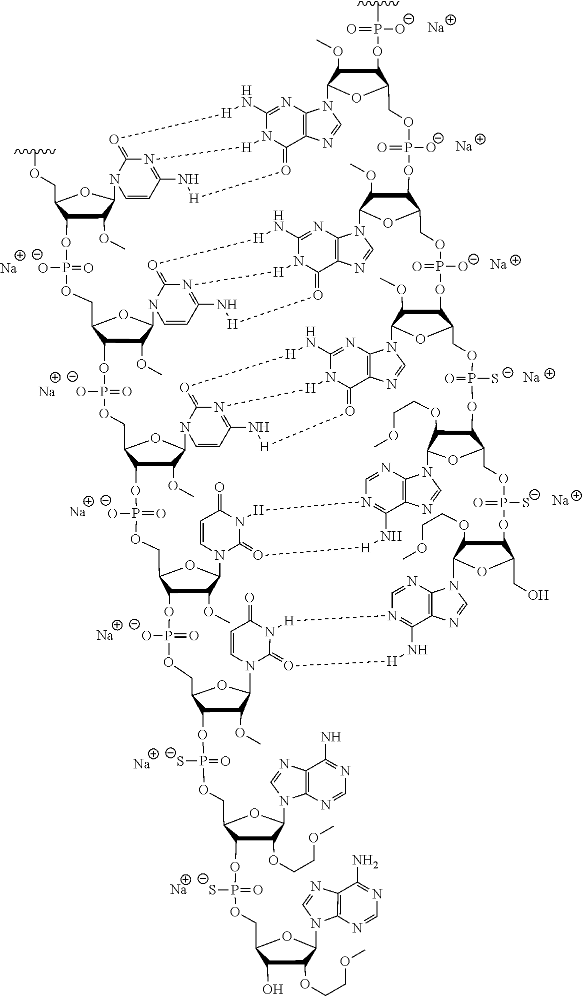

### (SEQ ID NO: 31 and SEQ ID NO: 79).

h. Compound No. 1758231

Provided herein is Compound No. 1758231, which is an oligomeric duplex that consists of a first oligomeric compound containing a first modified oligonucleotide and a second oligomeric compound containing a second modified oligonucleotide attached to a conjugate group as follows.

### First Oligomeric Compound of Compound No. 1758231:

The first modified oligonucleotide of Compound no. 1758231, which is Compound No. 1757481, has a nucleobase sequence of (from 5′ to 3′) TCACUGAGAAUACTGTCCCUUAA (SEQ ID NO: 4), wherein each of nucleosides 1, 9, 22 and 23 (from 5′ to 3′) comprises a 2′-MOE modified sugar moiety, nucleoside 2 (from 5′ to 3′) comprises a 2′-fluoro modified sugar moiety, each of nucleosides 3-5, 7, 8, 10-13, 15 and 17-21 (from 5′ to 3′) comprises a 2′-OMe modified sugar moiety, and each of nucleosides 6, 14 and 16 (from 5′ to 3′) is a 2′-deoxynucleoside, wherein each of the internucleoside linkages linking nucleosides 1 to 2, 2 to 3, 6 to 7, 14 to 15, 16 to 17, 21 to 22, and 22 to 23 (from 5′ to 3′) is a phosphorothioate internucleoside linkage and each of the internucleoside linkages linking nucleosides 3 to 4, 4 to 5, 5 to 6, 7 to 8, 8 to 9, 9 to 10, 10 to 11, 11 to 12, 12 to 13, 13 to 14, 15 to 16, 17 to 18, 18 to 19, 19 to 20, and 20 to 21 (from 5′ to 3′) is a phosphodiester linkage, and wherein nucleoside 1 (from 5′ to 3′) is attached to a vinyl phosphonate moiety.

The first oligomeric compound of Compound 1758231 is represented by the following chemical notation: VP-TₑₛC_{fs}A_{yo}C_{yo}U_{yo}G_{ds}A_{yo}G_{yo}AₑₒA_{yo}U_{yo}A_{yo}C_{yo}T_{ds}G_{yo}T_{ds}C_{yo}C_{yo}C_{yo}U_{yo}U_{ys}AₑₛAₑ (SEQ ID NO: 31); wherein
A=an adenine nucleobase,
C=a cytosine nucleobase,
G=a guanine nucleobase,
T=a thymine nucleobase,
U=a uracil nucleobase,
d=a 2′-β-D-deoxyribosyl sugar moiety,
e=a 2′-MOE sugar moiety,
f=a 2′-fluoro sugar moiety,
y=a 2′-OMe sugar moiety,
o=a phosphodiester internucleoside linkage,
s=a phosphorothioate internucleoside linkage, and
VP=a 5′ vinyl phosphonate moiety.

### Second Oligomeric Compound of Compound No. 1758231:

The second oligomeric compound of Compound No. 1758231, which is Compound No. 1735443, has a nucleobase sequence of (from 5′ to 3′) AAGGGACAGUAUUCUCAGUGA (SEQ ID NO: 6), wherein each of nucleosides 1, 2, 20 and 21 (from 5′ to 3′) comprises a 2′-MOE modified sugar moiety, each of nucleosides 10 and 11 (from 5′ to 3′) comprises a 2′-fluoro modified sugar moiety, and each of nucleosides 3-9, and 12-19 (from 5′ to 3′) comprises a 2′-OMe modified sugar moiety, wherein each of the internucleoside linkages linking nucleosides 1 to 2, 2 to 3, 10 to 11, 19 to 20, and 20 to 21 (from 5′ to 3′) is a phosphorothioate internucleoside linkage and each of the internucleoside linkages linking nucleosides 3 to 4, 4 to 5, 5 to 6, 6 to 7, 7 to 8, 8 to 9, 9 to 10, 11 to 12, 12 to 13, 13 to 14, 14 to 15, 15 to 16, 16 to 17, 17 to 18, and 18 to 19 (from 5′ to 3′) is a phosphodiester linkage, and wherein nucleoside 1 (from 5′ to 3′) is attached is attached through the 5′-OH of the oligonucleotide to a conjugate group comprising [THA-GalNAc].

The second oligomeric compound of Compound 1758231 is represented by the following chemical notation: [THA-GalNAc]-AₑₛAₑₛG_{yo}G_{yo}G_{yo}A_{yo}C_{yo}A_{yo}G_{yo}U_{fs}A_{fo}U_{yo}U_{yo}C_{yo}U_{yo}C_{yo}A_{yo}G_{yo}U_{ys}GₑₛAₑ (SEQ ID NO: 40); wherein:

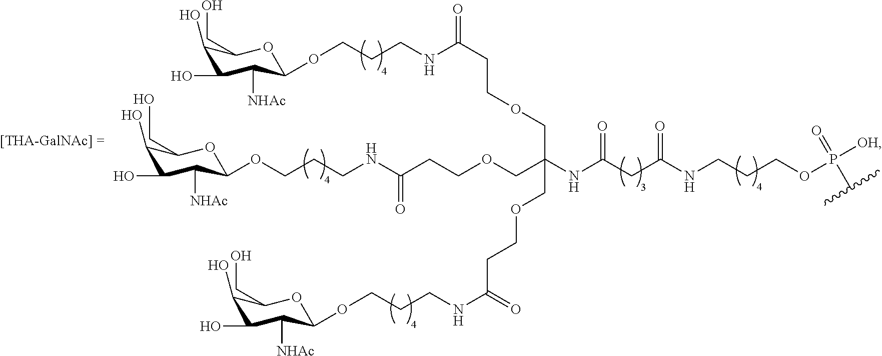

A=an adenine nucleobase,
C=a cytosine nucleobase,
G=a guanine nucleobase,
T=a thymine nucleobase,
U=a uracil nucleobase,
e=a 2′-MOE sugar moiety,
f=a 2′-fluoro sugar moiety,
y=a 2′-OMe sugar moiety,
o=a phosphodiester internucleoside linkage, and
s=a phosphorothioate internucleoside linkage.

The following chemical structure is one structural representation of Compound No. 1758231:

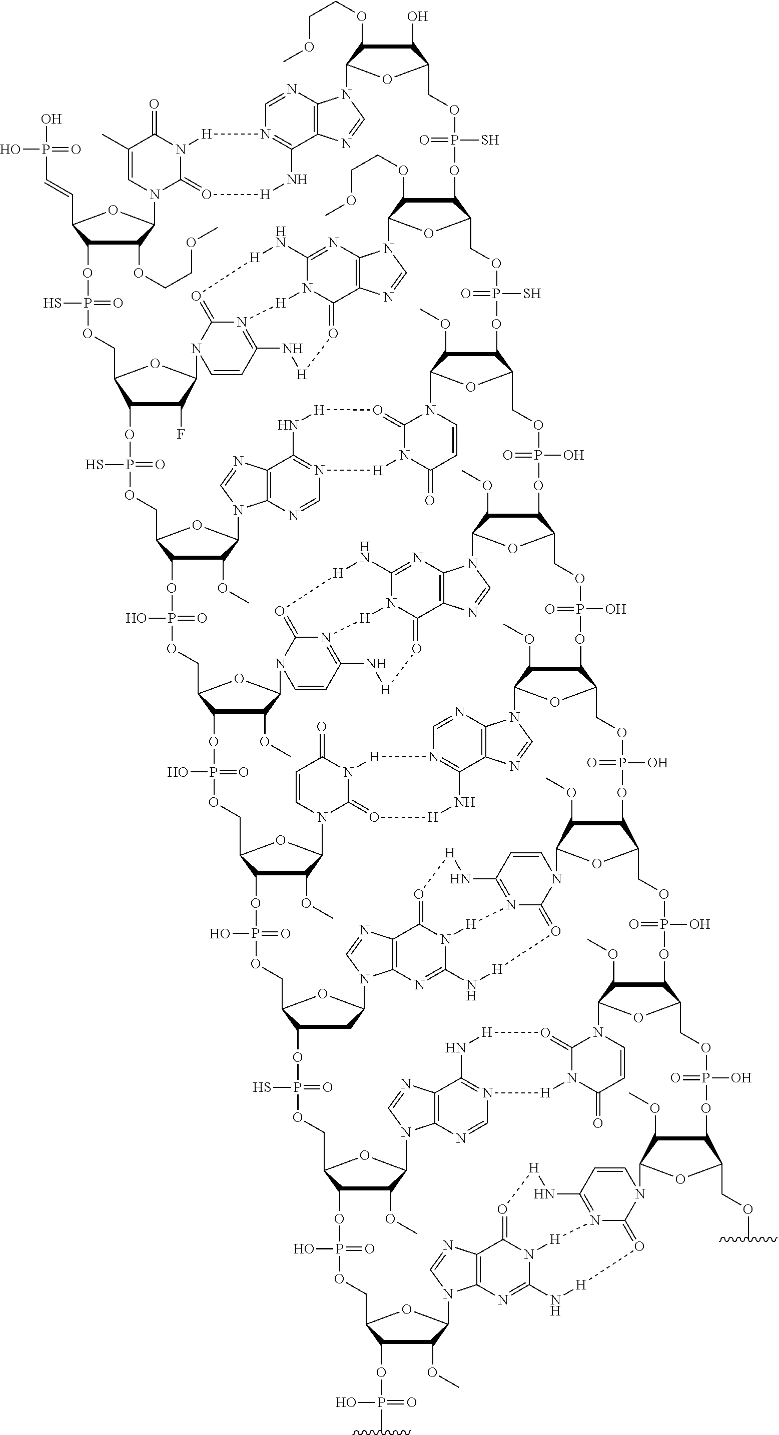

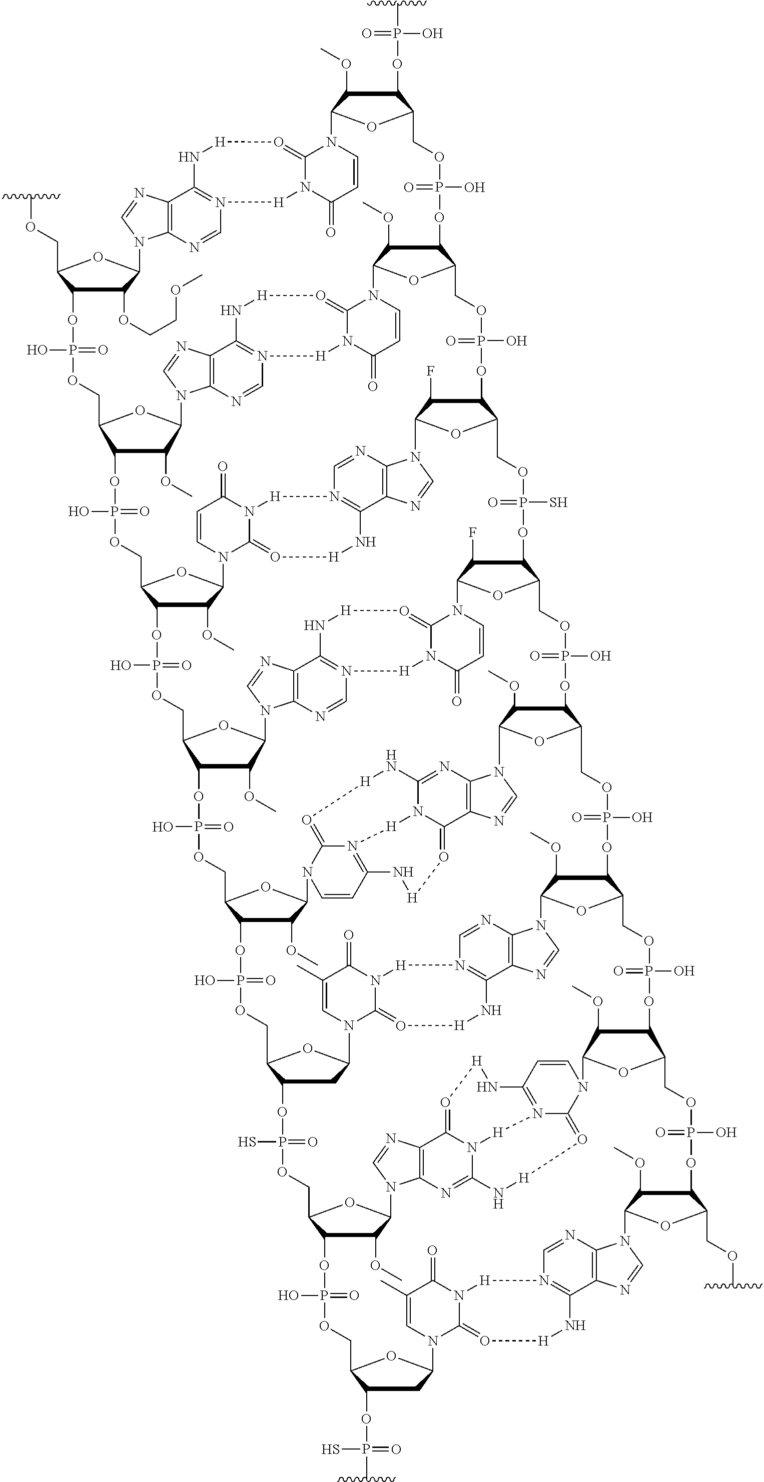

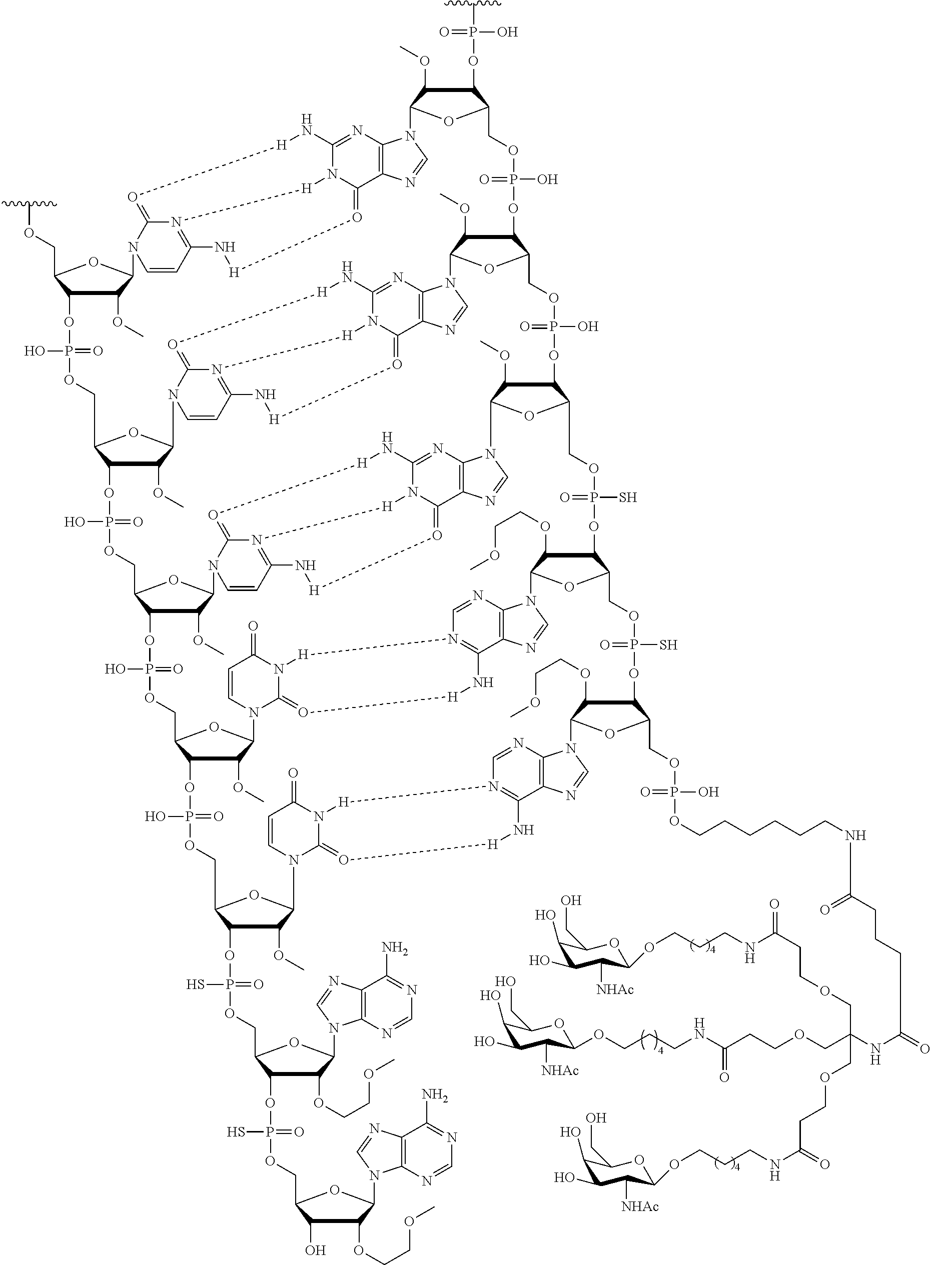

(SEQ ID NO: 31 and SEQ ID NO: 40), or an ion or salt thereof.

In certain embodiments, Compound No. 1758231 is in the form of an anion or a salt thereof, for example, the oligomeric duplex may be in the form of a sodium salt. In certain embodiments, the oligomeric duplex is in anionic form in a solution. In certain embodiments, Compound No. 1758231 is a sodium salt or a potassium salt.

The following chemical structure represents Compound No. 1758231 in sodium solution:

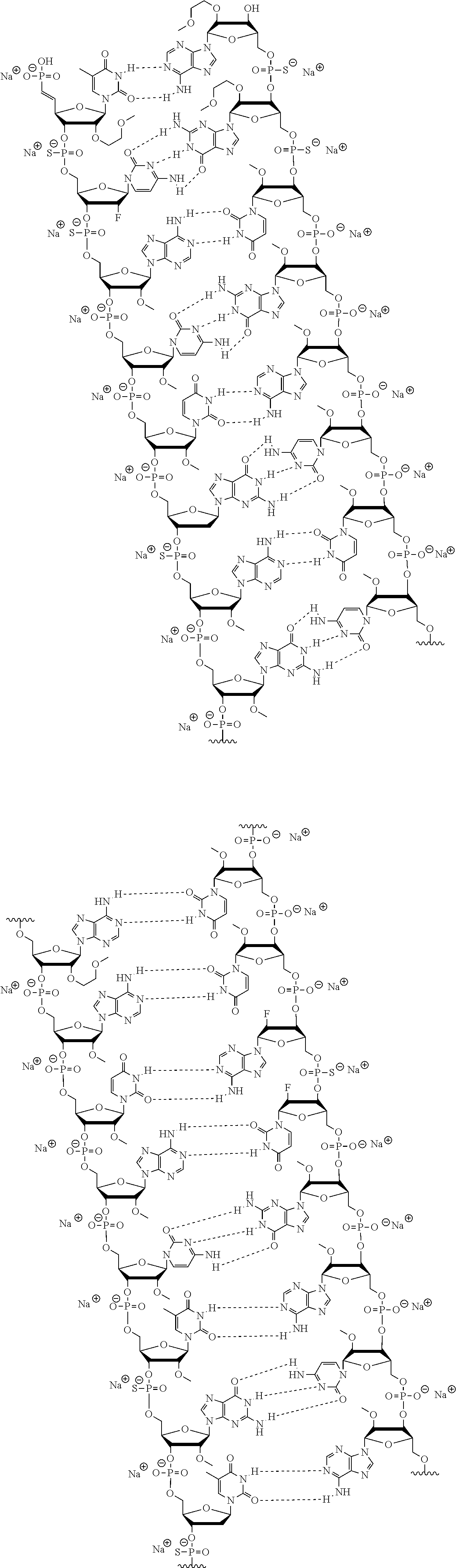

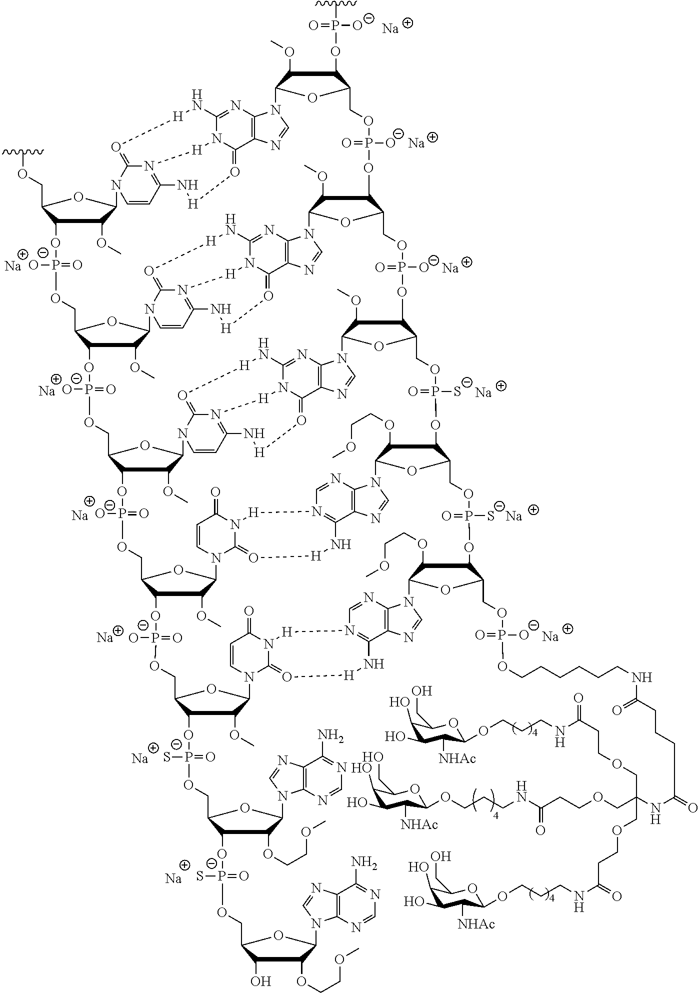

### (SEQ ID NO: 31 and SEQ ID NO: 40).

In certain embodiments, provided herein are oligomeric duplexes comprising or consisting of a first oligomeric compound containing a first modified oligonucleotide and a second oligomeric compound containing a second modified oligonucleotide as follows.

The first modified oligonucleotide is Compound No. 1757481, as described in h. above for Compound no. 1758231.

The second oligomeric compound has a nucleobase sequence of (from 5′ to 3′) AAGGGACAGUAUUCUCAGUGA (SEQ ID NO: 6), wherein each of nucleosides 1, 2, 20 and 21 (from 5′ to 3′) comprises a 2′-MOE modified sugar moiety, each of nucleosides 10 and 11 (from 5′ to 3′) comprises a 2′-fluoro modified sugar moiety, and each of nucleosides 3-9, and 12-19 (from 5′ to 3′) comprises a 2′-OMe modified sugar moiety, wherein each of the internucleoside linkages linking nucleosides 1 to 2, 2 to 3, 10 to 11, 19 to 20, and 20 to 21 (from 5′ to 3′) is a phosphorothioate internucleoside linkage and each of the internucleoside linkages linking nucleosides 3 to 4, 4 to 5, 5 to 6, 6 to 7, 7 to 8, 8 to 9, 9 to 10, 11 to 12, 12 to 13, 13 to 14, 14 to 15, 15 to 16, 16 to 17, 17 to 18, and 18 to 19 (from 5′ to 3′) is a phosphodiester linkage.

The second oligomeric compound is represented by the following chemical notation: AₑₛAₑₛG_{yo}G_{yo}G_{yo}A_{yo}C_{yo}A_{yo}G_{yo}U_{fs}A_{fo}U_{yo}U_{yo}C_{yo}U_{yo}C_{yo}A_{yo}G_{yo}U_{ys}GₑₛAₑ (SEQ ID NO: 80); wherein:
A=an adenine nucleobase,
C=a cytosine nucleobase,
G=a guanine nucleobase,
T=a thymine nucleobase,
U=a uracil nucleobase,
e=a 2′-MOE sugar moiety,
f=a 2′-fluoro sugar moiety,
y=a 2′-OMe sugar moiety,
o=a phosphodiester internucleoside linkage, and
s=a phosphorothioate internucleoside linkage.

In certain embodiments, such an oligomeric duplex is represented by the following chemical structure:

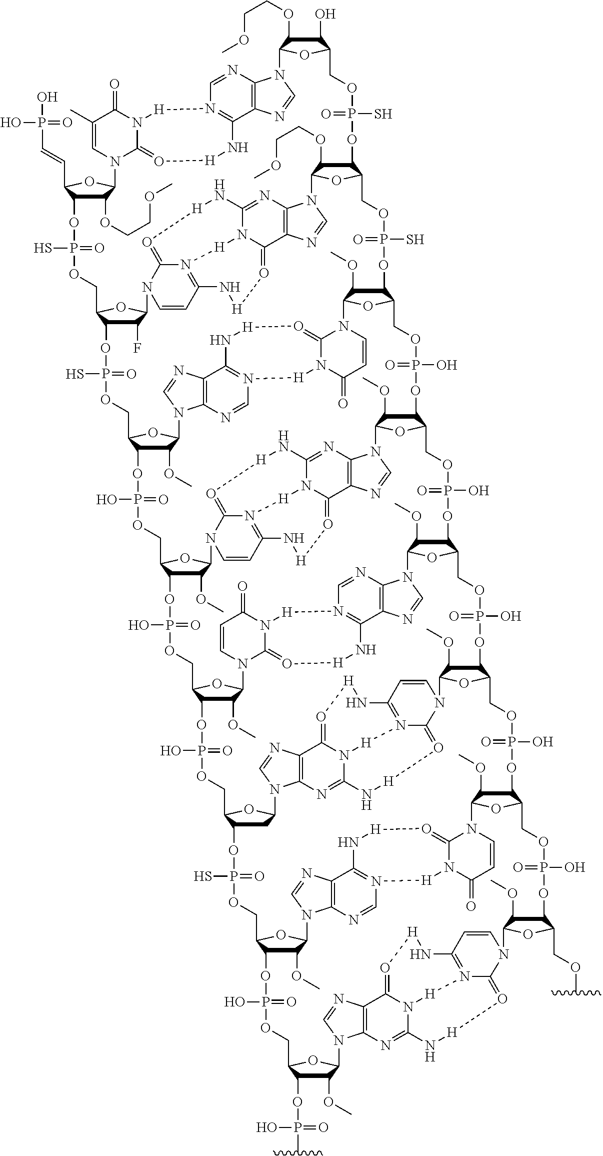

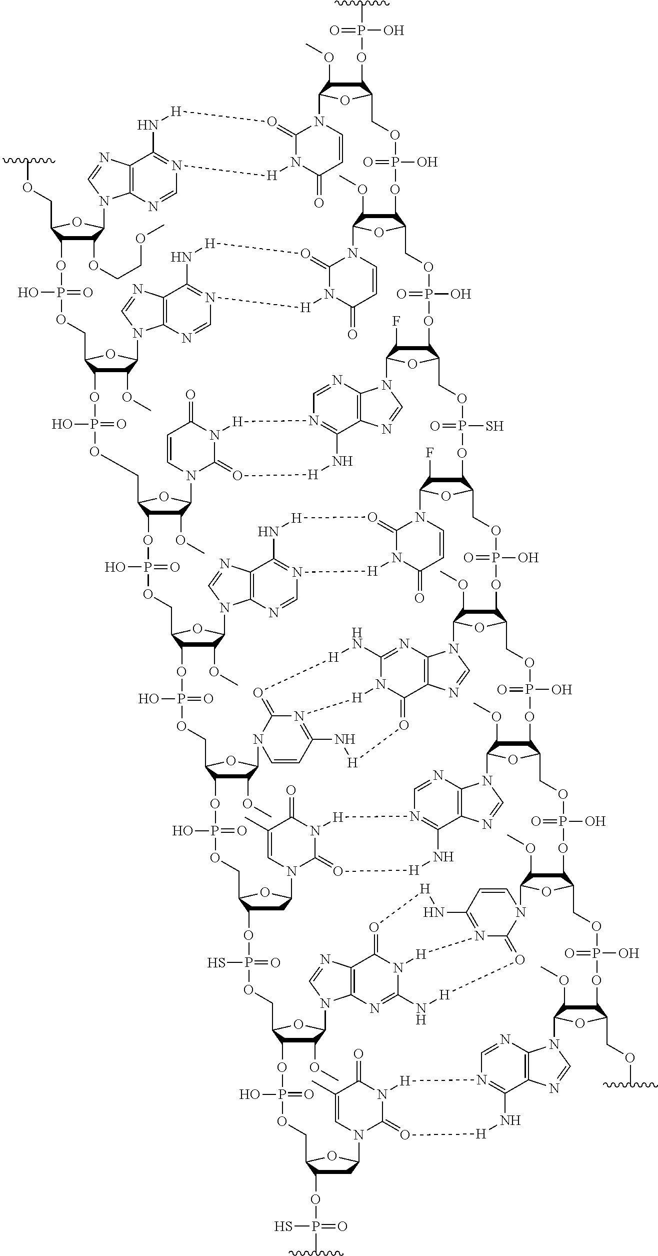

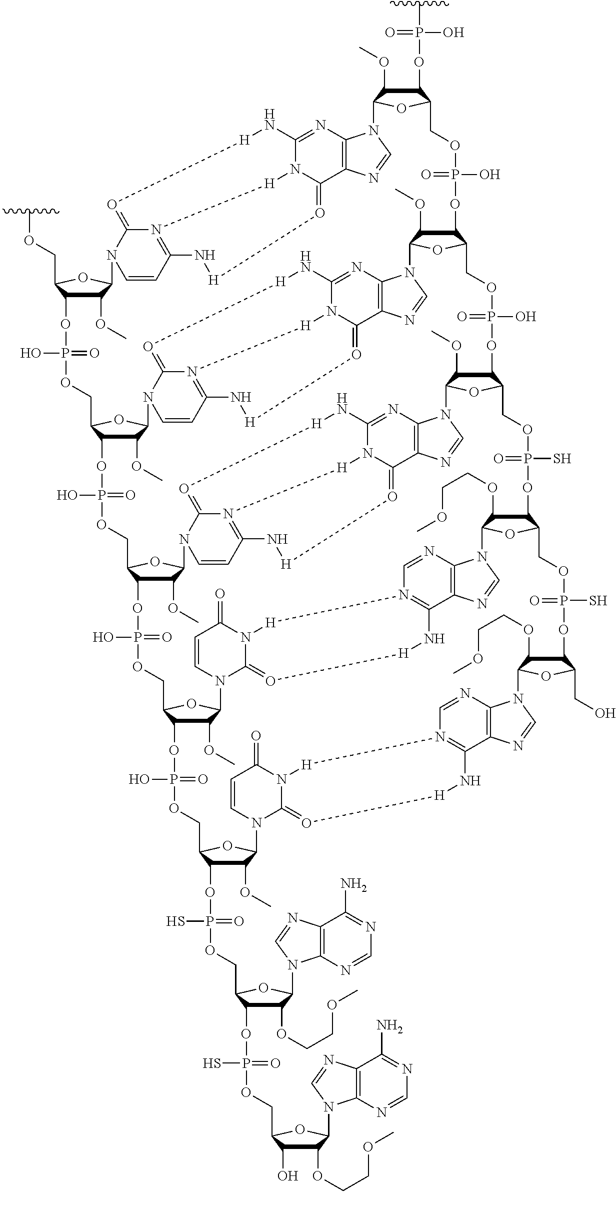

(SEQ ID NO: 31 and SEQ ID NO: 80), or an ion or salt thereof.

In certain embodiments, such an oligomeric duplex is in the form of an anion or a salt thereof, for example, a sodium salt. In certain embodiments, the oligomeric duplex is in anionic form in a solution. In certain embodiments, the oligomeric duplex is a sodium salt or a potassium salt.

The following chemical structure represents such an oligomeric duplex in sodium solution:

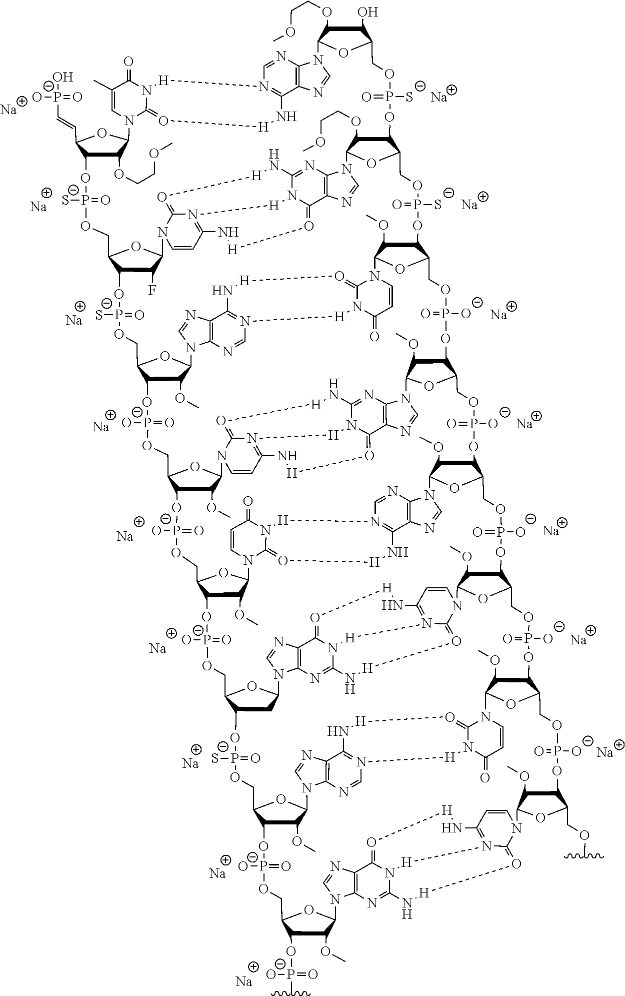

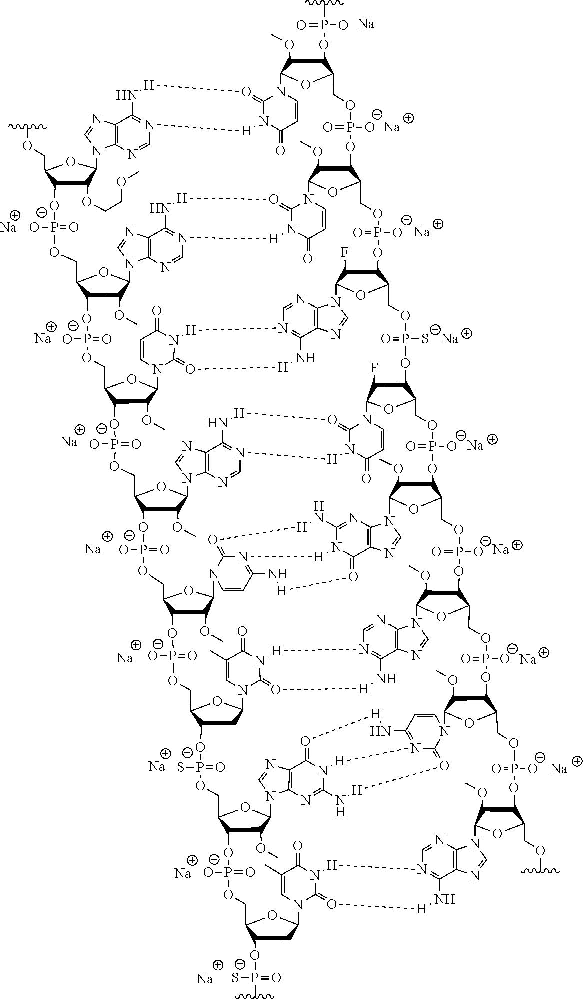

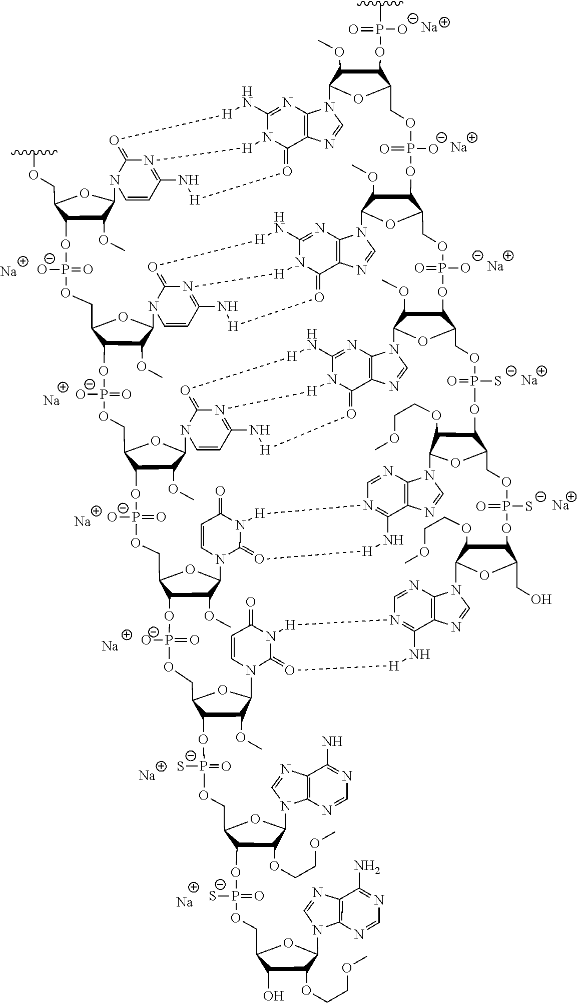

### (SEQ ID NO: 31 and SEQ ID NO: 80).

### Nonlimiting Disclosure and Incorporation by Reference

Each of the literature and patent publications listed herein is incorporated by reference in its entirety.

While certain compounds, compositions and methods described herein have been described with specificity in accordance with certain embodiments, the following examples serve only to illustrate the compounds described herein and are not intended to limit the same. Each of the references, GenBank accession numbers, ENSEMBL identifiers, and the like recited in the present application is incorporated herein by reference in its entirety.

Although the sequence listing accompanying this filing identifies each sequence as either “RNA” or “DNA” as required, in reality, those sequences may be modified with any combination of chemical modifications. One of skill in the art will readily appreciate that such designation as “RNA” or “DNA” to describe modified oligonucleotides is, in certain instances, arbitrary. For example, an oligonucleotide comprising a nucleoside comprising a 2′-OH sugar moiety and a thymine base could be described as a DNA having a modified sugar (2′-OH in place of one 2′-H of DNA) or as an RNA having a modified base (thymine (methylated uracil) in place of an uracil of RNA). Accordingly, nucleic acid sequences provided herein, including, but not limited to those in the sequence listing, are intended to encompass nucleic acids containing any combination of natural or modified RNA and/or DNA, including, but not limited to such nucleic acids having modified nucleobases. By way of further example and without limitation, an oligomeric compound having the nucleobase sequence “ATCGATCG” encompasses any oligomeric compounds having such nucleobase sequence, whether modified or unmodified, including, but not limited to, such compounds comprising RNA bases, such as those having sequence “AUCGAUCG” and those having some DNA bases and some RNA bases such as “AUCGATCG” and oligomeric compounds having other modified nucleobases, such as “ATmCGAUCG,” wherein ^{m}C indicates a cytosine base comprising a methyl group at the 5-position.

Certain compounds described herein (e.g., modified oligonucleotides) have one or more asymmetric center and thus give rise to enantiomers, diastereomers, and other stereoisomeric configurations that may be defined, in terms of absolute stereochemistry, as (R) or (S), as a or R such as for sugar anomers, or as (D) or (L), such as for amino acids, etc. Compounds provided herein that are drawn or described as having certain stereoisomeric configurations include only the indicated compounds. Compounds provided herein that are drawn or described with undefined stereochemistry include all such possible isomers, including their stereorandom and optically pure forms, unless specified otherwise. Likewise, tautomeric forms of the compounds herein are also included unless otherwise indicated. Unless otherwise indicated, compounds described herein are intended to include corresponding salt forms.

The compounds described herein include variations in which one or more atoms are replaced with a non-radioactive isotope or radioactive isotope of the indicated element. For example, compounds herein that comprise hydrogen atoms encompass all possible deuterium substitutions for each of the ¹H hydrogen atoms. Isotopic substitutions encompassed by the compounds herein include but are not limited to: ²H or ³H in place of ¹H, ¹³C or ¹⁴C in place of ¹²C, ¹⁵N in place of ¹⁴N, ¹⁷O or ¹⁸O in place of ¹⁶O and ³³S, ³⁴S, ³⁵S, or ³⁶S in place of ³²S. In certain embodiments, non-radioactive isotopic substitutions may impart new properties on the oligomeric agent that are beneficial for use as a therapeutic or research tool. In certain embodiments, radioactive isotopic substitutions may make the compound suitable for research or diagnostic purposes such as imaging.

### EXEMPLIFICATION

The following examples illustrate certain embodiments of the present disclosure and are not limiting. Moreover, where specific embodiments are provided, the inventors have contemplated generic application of those specific embodiments. For example, disclosure of an oligonucleotide having a particular motif provides reasonable support for additional oligonucleotides having the same or similar motif And, for example, where a particular high-affinity modification appears at a particular position, other high-affinity modifications at the same position are considered suitable, unless otherwise indicated.

### Example 1: Design of Modified Oligomeric Agents that Target Human APOC3

Oligomeric agents comprising antisense oligomeric compounds complementary to a human APOC3 nucleic acid, and sense oligomeric compounds complementary to antisense oligomeric compounds, as well as oligomeric duplexes comprising antisense and sense oligomeric compounds, were designed as follows.

### Design of Antisense Oligomeric Compounds

Antisense oligomeric compounds in Tables 1-4 are 23 (Tables 1, 2 and 3) or 21 (Table 4) nucleosides in length; have a sugar motif as designated in the “Sugar Motif (5′ to 3′)” column, wherein each ‘y’ represents a ribo-2′-OMe sugar moiety, each ‘f’ represents a ribo-2′-F sugar moiety, each ‘e’ represents a ribo-2′-MOE sugar moiety, and each ‘d’ represents a 2′-β-D-deoxyribosyl sugar moiety, each ‘k’ represents a cEt sugar moiety, and each ‘[FHNA]’ represents a 3′-fluoro hexitol sugar moiety; and have an internucleoside linkage motif as designated in the “Internucleoside Linkage (5′ to 3′)” column, wherein each ‘o’ represents a phosphodiester internucleoside linkage, each ‘s’ represents a phosphorothioate internucleoside, linkage, each ‘z’ represents a mesyl phosphoramidate linkage, and each ‘x’ represents a methoxypropyl phosphonate linkage. Each cytosine residue is a non-methylated cytosine. Each antisense oligomeric compound in Table 1, 3 and 4 has a vinyl phosphonate (VP—) moiety on the 5′-end. The antisense oligomeric compound in Table 2 has a methylene phosphonate (mP-) moiety on the 5′-end.

Each antisense oligomeric compound listed in Tables 1-4 is complementary to SEQ ID NO: 1 (GENBANK Accession No. NM_000040.2), except for a single mismatch at position 1 on the 5′ end of the antisense oligomeric compound in each of Tables 1-4; two additional mismatches at positions 22 and 23 on the 3′-end of the antisense oligomeric compounds in Table 3; and two additional mismatches at positions 20 and 21 on the 3′-end of the antisense oligomeric compounds in Table 4. The nucleobase sequence TCACUGAGAAUACUGUCCCUUUU (SEQ ID NO:2) and TCACUGAGAAUACUGUCCCUUTT (SEQ ID NO: 110) in Tables 1 and 2 has a start site of 450 and a stop site of 471 on SEQ ID NO: 1. The nucleobase sequences TCACUGAGAAUACUGUCCCUUAA (SEQ ID NO: 3) and TCACUGAGAAUACUGUCCCTTAA (SEQ ID NO: 103) and TCACUGAGAAUACTGTCCCUUAA (SEQ ID NO: 4) in Table 3 have a start site of 452 and a stop site of 471 on SED ID NO: 1. The nucleobase sequence TCACUGAGAAUACUGUCCCAA (SEQ ID NO:5) in Table 4 has a start site of 454 and a stop site of 471 on SEQ ID NO: 1. “Start site” is the 5′-most nucleoside; and “stop site” indicates the 3′-most nucleoside to which the antisense oligomeric compound is complementary in the target nucleic acid sequence SEQ ID NO: 1.

TABLE 1
5′ vinyl phosphonate antisense oligomeric compounds targeted to human APOC3

| Compound | Nucleobase Sequence | Sugar Motif | Internucleoside Linkage | SEQ |
|---|---|---|---|---|
| No. | (5′ to 3′) | (5′ to 3′) | (5′ to 3′) | ID NO |
| 1692954 | TCACUGAGAAUACUGUCCCUUUU | efyyyfyyyyyyyfyfyyyyyyy | ssooooooooooooooooooss | 11 |
| 1704067 | TCACUGAGAAUACUGUCCCUUUU | efyyydyyyyyyyfyfyyyyyyy | ssooooooooooooooooooss | 12 |
| 1707299 | TCACUGAGAAUACUGUCCCUUUU | efyyydyyyyyyyfyfyyyyyyy | ssooosooooooooooooooss | 13 |
| 1712886 | TCACUGAGAAUACUGUCCCUUUU | efyyydyyyyyyydydyyyyyyy | ssooooooooooooooooooss | 14 |
| 1712887 | TCACUGAGAAUACUGUCCCUUUU | efyyydyyyyyyydydyyyyyyy | ssooosooooooososooooss | 15 |
| 1718686 | TCACUGAGAAUACUGUCCCUUUU | efyyyyyyyyyyyfyyyyyyyyy | ssooooooooooooooooooss | 16 |
| 1732454 | TCACUGAGAAUACUGUCCCUUUU | efyyyfyyeyyyyfyfyyyyyyy | ssooooooooooooooooooss | 17 |
| 1738136 | TCACUGAGAAUACUGUCCCUUUU | efyyyfyyeeyyyfyfyyyyyyy | ssooooooooooooooooooss | 18 |
| 1692965 | TCACUGAGAAUACUGUCCCUUUU | efyyyfyyyyyyyfyfyyyyyyy | zzoooooooooooooooooozs | 96 |
| 1692968 | TCACUGAGAAUACUGUCCCUUUU | efyyyfyyyyyyy[FHNA]yfyy | ssooooooooooooooooooss | 97 |
| | | yyyyy | | |
| 1707309 | TCACUGAGAAUACUGUCCCUUUU | efyyydyyyyyyyfyfyyyyyyy | szooosooooooooooooooss | 98 |
| 1707311 | TCACUGAGAAUACUGUCCCUUUU | efyyydyyyyyyyfyfyyyyyyy | szooosooooooozozooooss | 99 |
| 1718714 | TCACUGAGAAUACUGUCCCUUTT | efyyyyyyyyyyyfyyyyyyyee | ssooooooooooooooooooss | 100 |
| 1718722 | TCACUGAGAAUACUGUCCCUUTT | efyyyyyyyyyyyfyyyyyyykk | ssooooooooooooooooooss | 101 |

TABLE 2
5′ methylene phosphonate antisense oligomeric compounds targeted to human APOC3

| Compound | Nucleobase Sequence | Sugar Motif | Internucleoside Linkage | SEQ |
|---|---|---|---|---|
| No. | (5′ to 3′) | (5′ to 3′) | (5′ to 3′) | ID NO |
| 1726615 | TCACUGAGAAUACUGUCCCUUUU | efyyyfyyyyyyyfyfyyyyyyy | ssooooooooooooooooooss | 19 |

TABLE 3
5′ vinyl phosphonate antisense oligomeric compounds targeted to human APOC3

| Compound | Nucleobase Sequence | Sugar Motif | Internucleoside Linkage | SEQ |
|---|---|---|---|---|
| No. | (5′ to 3′) | (5′ to 3′) | (5′ to 3′) | ID NO |
| 1735442 | TCACUGAGAAUACUGUCCCUUAA | efyyydyyyyyyyfyfyyyyyee | ssooosooooooooooooooss | 20 |
| 1736491 | TCACUGAGAAUACUGUCCCUUAA | efyyydyyyyyyydydyyyyyee | ssooosooooooososooooss | 21 |
| 1744806 | TCACUGAGAAUACUGUCCCUUAA | efyyydyyyyyyyfyfyyyyyee | ssooooooooooooooooooss | 22 |
| 1744807 | TCACUGAGAAUACUGUCCCUUAA | efyyydyyyyyyydydyyyyyee | ssooooooooooooooooooss | 23 |
| 1752160 | TCACUGAGAAUACUGUCCCUUAA | efyyydyyeyyyyfyfyyyyyee | ssooosooooooooooooooss | 24 |
| 1752677 | TCACUGAGAAUACUGUCCCUUAA | efyyydyyeyyyydydyyyyyee | ssooooooooooooooooooss | 25 |
| 1752680 | TCACUGAGAAUACUGUCCCUUAA | efyyydyyeeyyydydyyyyyee | ssooooooooooooooooooss | 26 |
| 1752688 | TCACUGAGAAUACUGUCCCUUAA | efyyydyyeyyyyfyfyyyyyee | ssooooooooooooooooooss | 27 |
| 1753166 | TCACUGAGAAUACTGTCCCUUAA | efyyydyyeyyyydydyyyyyee | ssooooooooooooooooooss | 28 |
| 1753167 | TCACUGAGAAUACUGUCCCUUAA | efyyydyyeyyyydydyyyyyee | ssooosooooooososooooss | 29 |
| 1753787 | TCACUGAGAAUACUGUCCCUUAA | efyyydyyeeyyyfyfyyyyyee | ssooooooooooooooooooss | 30 |
| 1757481 | TCACUGAGAAUACTGTCCCUUAA | efyyydyyeyyyydydyyyyyee | ssooosooooooososooooss | 31 |
| 1736598 | TCACUGAGAAUACUGUCCCTTAA | efyyydyyyyyyyfyfyyydkee | ssooosooooooooooooosss | 102 |

TABLE 4
5′ vinyl phosphonate antisense oligomeric compounds targeted to human APOC3

| Compound | Nucleobase Sequence | Sugar Motif | Internucleoside Linkage | SEQ |
|---|---|---|---|---|
| No. | (5′ to 3′) | (5′ to 3′) | (5′ to 3′) | ID NO |
| 1669175 | TCACUGAGAAUACUGUCCCAA | efyyyfyyyyyyyfyfyyyee | ssooooooooooooooooss | 32 |
| 1724017 | TCACUGAGAAUACUGUCCCAA | efyyydyyyyyyyfyfyyyee | ssooosooooooooooooss | 33 |
| 1736515 | TCACUGAGAAUACUGUCCCAA | efyyydyyyyyyydydyyyee | ssooosooooooososooss | 34 |

### Design of Sense Oligomeric Compounds

Sense oligonucleotides in Tables 5-8 below are each 21 nucleosides (Tables 5 and 8), 19 nucleosides (Table 6), or 22 nucleosides (Table 7) in length, have a sugar motif as designated in the “Sugar Motif (5′ to 3′)” column, wherein each ‘y’ represents a ribo-2′-OMe sugar moiety, each ‘f’ represents a ribo-2′-F sugar moiety, each ‘e’ represents a ribo-2′-MOE sugar moiety, each ‘k’ represents a cEt sugar moiety, and each ‘[FHNA]’ represents a 3′-fluoro hexitol sugar moiety and each ‘d’ represents a 2′-β-D-deoxyribosyl sugar moiety; and have an internucleoside linkage motif as designated in the “Internucleoside Linkage (5′ to 3′)” column, wherein each ‘o’ represents a phosphodiester internucleoside linkage, each ‘z’ represents a mesyl phosphoramidate internucleoside linkage and each ‘s’ represents a phosphorothioate internucleoside linkage. Each cytosine residue is non-methylated. The nucleobase sequence AAGGGACAGUAUUCUCAGUGA (SEQ ID NO: 6) of oligonucleotides in Table 5 and Table 8 is complementary to an antisense oligomeric compound, wherein the last two 3′-nucleosides of the antisense oligomeric compound are not paired with the sense oligomeric compound, rather are overhanging nucleosides. The nucleobase sequence GGGACAGUAUUCUCAGUGA (SEQ ID NO: 7) of oligonucleotides in Table 6 is complementary to an antisense oligomeric compound, wherein the last two 3′-nucleosides of the antisense oligomeric compound are not paired with the sense oligomeric compound, rather are overhanging nucleosides. The nucleobase sequence TAAGGGACAGUAUUCUCAGUGA (SEQ ID NO: 8) of oligonucleotides in Table 7 is complementary to an antisense oligomeric compound, wherein the last 3′-nucleoside of the antisense oligomeric compound is not paired with the sense oligomeric compound, rather is an overhanging nucleoside. Each sense oligomeric compound in Tables 5-7 is conjugated to a THA-GalNAc conjugate group attached to the 5′-OH of the oligonucleotide. The structure of THA-GalNAc is:

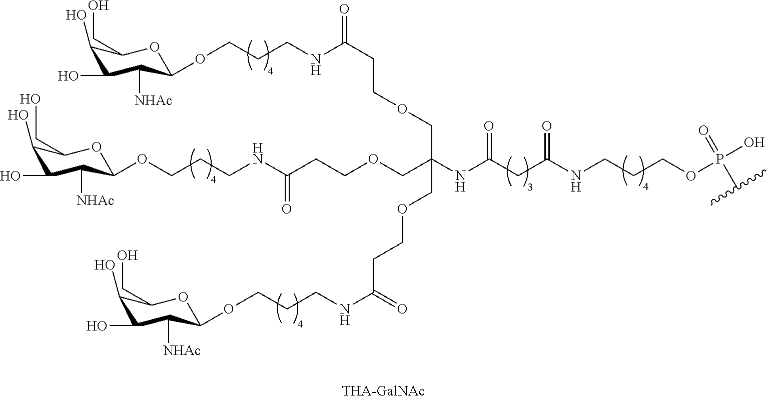

The sense oligomeric compound in Table 8 is conjugated to a HPPO-GalNAc conjugate group attached to the 3′-OH of the oligonucleotide. The structure of HPPO-GalNAc is:

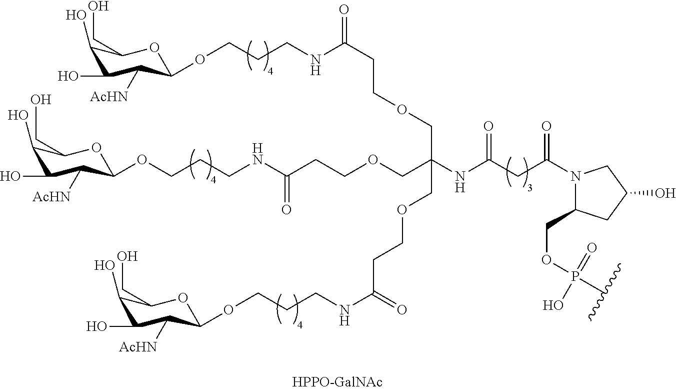

TABLE 5
5′ GalNAc conjugated sense oligomeric compounds

| Compound | Nucleobase Sequence | Sugar Motif | Internucleoside | SEQ |
|---|---|---|---|---|
| No. | (5′ to 3′) | (5′ to 3′) | Linkage (5′ to 3′) | ID NO |
| 1692955 | AAGGGACAGUAUUCUCAGUGA | yyyyyyfyfffyyyyyyyyyy | ssooooooooooooooooss | 35 |
| 1704068 | AAGGGACAGUAUUCUCAGUGA | yyyyyyyyyffyyyyyyyyyy | ssooooooooooooooooss | 36 |
| 1704093 | AAGGGACAGUAUUCUCAGUGA | yyyyyyyfyfyfyyyyyyyyy | ssooooooooooooooooss | 37 |
| 1707308 | AAGGGACAGUAUUCUCAGUGA | yyyyyyyyyffyyyyyyyyyy | ssooooooosooooooooss | 38 |
| 1718715 | AAGGGACAGUAUUCUCAGUGA | eeyyyyyyyffyyyyyyyyee | ssooooooooooooooooss | 39 |
| 1735443 | AAGGGACAGUAUUCUCAGUGA | eeyyyyyyyffyyyyyyyyee | ssooooooosooooooooss | 40 |
| 1736494 | AAGGGACAGUAUUCUCAGUGA | eeyyyyyyffyyyyyyyyyee | ssooooooosooooooooss | 41 |
| 1692969 | AAGGGACAGUAUUCUCAGUGA | yyyyyy[FHNA]yff[FHNA] | ssooooooooooooooooss | 104 |
| | | yyyyyyyyyy | | |
| 1737376 | AAGGGACAGUAUUCUCAGUGA | yyyyyyyykdkyyyyyyyyyy | ssooooooooooooooooss | 105 |
| 1729192 | AAGGGACAGUAUUCUCAGUGA | yyyyyyyyyyyyyyyyyyyyy | ssooooooooooooooooss | 106 |
| 1707310 | AAGGGACAGUAUUCUCAGUGA | yyyyyyyyyffyyyyyyyyyy | ssooooooozooooooooss | 111 |

TABLE 6
5′ GalNAc conjugated sense oligomeric compounds

| Compound | Nucleobase Sequence | Sugar Motif | Internucleoside Linkage | SEQ ID |
|---|---|---|---|---|
| No. | (5′ to 3′) | (5′ to 3′) | (5′ to 3′) | NO |
| 1669177 | GGGACAGUAUUCUCAGUGA | yyyyyyfyfffyyyyyyyy | ssooooooooooooooss | 42 |
| 1723880 | GGGACAGUAUUCUCAGUGA | yyyyyyfyfyyyyyyyyyy | ssooooooooooooooss | 43 |
| 1723881 | GGGACAGUAUUCUCAGUGA | yyyyyyyfyfyyyyyyyyy | ssooooooooooooooss | 44 |
| 1723882 | GGGACAGUAUUCUCAGUGA | yyyyyyyyfyfyyyyyyyy | ssooooooooooooooss | 45 |
| 1735459 | GGGACAGUAUUCUCAGUGA | eeyyyyyyyffyyyyyyee | ssooooooosooooooss | 46 |
| 1737317 | GGGACAGUAUUCUCAGUGA | eeyyyyyyffyyyyyyyee | ssoooooosoooooooss | 47 |

TABLE 7
5′ GalNAc conjugated sense oligomeric compounds

| Compound | Nucleobase Sequence | Sugar Motif | Internucleoside Linkage | SEQ |
|---|---|---|---|---|
| No. | (5′ to 3′) | (5′ to 3′) | (5′ to 3′) | ID NO |
| 1706978 | TAAGGGACAGUAUUCUCAGUGA | dyyyyyyfyfffyyyyyyyyyy | ossooooooooooooooooss | 48 |

TABLE 8
3′ GalNAc conjugated sense oligomeric compound

| Compound | Sequence | Sugar Motif | Internucleoside Linkage | SEQ |
|---|---|---|---|---|
| No. | (5′ to 3′) | (5′ to 3′) | (5′ to 3′) | ID NO |
| 1706985 | AAGGGACAGUAUUCUCAGUGA | yyyyyyfyfffyyyyyyyyyy | ssooooooooooooooooss | 49 |

### Design of Oligomeric Duplexes

Oligomeric duplex compounds prepared with antisense oligomeric compound and corresponding sense oligomeric compound are listed in Table 9.

TABLE 9
Oligomeric duplexes targeted to human APOC3

| Compound Number | Antisense Compound No. | Sense Compound No. |
|---|---|---|
| 1692958 | 1692954 | 1692955 |
| 1692964 | 1669175 | 1669177 |
| 1704071 | 1704067 | 1704068 |
| 1704096 | 1704067 | 1704093 |
| 1714226 | 1692954 | 1706978 |
| 1714229 | 1692954 | 1706985 |
| 1714244 | 1707299 | 1704068 |
| 1714253 | 1707299 | 1707308 |
| 1714259 | 1707299 | 1707310 |
| 1717167 | 1712886 | 1704068 |
| 1717170 | 1712887 | 1704068 |
| 1718689 | 1718686 | 1704068 |
| 1718692 | 1718686 | 1707308 |
| 1722860 | 1718686 | 1718715 |
| 1723889 | 1669175 | 1723880 |
| 1723892 | 1669175 | 1723881 |
| 1723895 | 1669175 | 1723882 |
| 1731577 | 1726615 | 1692955 |
| 1732457 | 1732454 | 1692955 |
| 1735455 | 1735442 | 1735443 |
| 1735463 | 1724017 | 1735459 |
| 1738342 | 1736491 | 1735443 |
| 1738345 | 1736515 | 1735459 |
| 1738348 | 1735442 | 1736494 |
| 1738354 | 1736515 | 1737317 |
| 1743461 | 1732454 | 1735443 |
| 1743464 | 1738136 | 1692955 |
| 1743470 | 1738136 | 1735443 |
| 1744810 | 1744806 | 1718715 |
| 1754076 | 1752688 | 1692955 |
| 1754466 | 1752688 | 1718715 |
| 1754787 | 1752688 | 1735443 |
| 1754958 | 1752160 | 1692955 |
| 1754961 | 1752160 | 1718715 |
| 1754964 | 1752160 | 1735443 |
| 1754970 | 1753166 | 1692955 |
| 1754973 | 1753166 | 1718715 |
| 1754976 | 1753166 | 1735443 |
| 1755057 | 1752677 | 1718715 |
| 1755060 | 1752680 | 1718715 |
| 1755063 | 1753167 | 1718715 |
| 1755066 | 1752677 | 1735443 |
| 1755069 | 1752680 | 1735443 |
| 1755072 | 1753167 | 1735443 |
| 1755096 | 1753787 | 1718715 |
| 1755099 | 1753787 | 1735443 |
| 1757505 | 1744806 | 1735443 |
| 1757508 | 1744807 | 1735443 |
| 1758193 | 1757481 | 1718715 |
| 1758231 | 1757481 | 1735443 |
| 1692972 | 1692965 | 1692955 |
| 1692981 | 1692968 | 1692955 |
| 1692984 | 1692954 | 1692969 |
| 1706916 | 1692968 | 1692969 |
| 1718718 | 1718714 | 1718715 |
| 1718721 | 1718714 | 1704068 |
| 1718728 | 1718722 | 1704068 |
| 1743442 | 1736598 | 1692955 |
| 1743450 | 1736598 | 1735443 |
| 1743453 | 1692954 | 1737376 |
| 1714256 | 1707309 | 1707308 |
| 1714262 | 1707311 | 1707308 |
| 1714265 | 1707311 | 1707310 |
| 1731581 | 1692954 | 1729192 |

### Example 2: Effect of Oligomeric Duplexes Targeting Human APOC3 in APOC3 Transgenic Mice

APOC3 transgenic mice were used to determine effects of a single dose of oligomeric duplex compounds on human APOC3. The APOC3 transgenic mouse model was previously described in Reaven G M, Mondon C E, Chen Y D, Breslow J L. I-ypertriglyceridemic mice transgenic for the human apolipoprotein C-III gene are neither insulin resistant nor hyperinsulineric. J Lipid Res. 1994 May; 35(5):820-4. (PMID 8071604). APOC3 transgenic mice were divided into groups of 3-4 mice each. Each mouse received a single subcutaneous injection of oligomeric duplex compound at a dose of 1 mg/kg. One group of 3 mice received a single subcutaneous injection of PBS and served as a control group. Results from separate studies conducted under the same conditions are provided in Tables 10-25.

Compound No. 1738179, prepared as a comparator compound, is a structural and activity surrogate for previously disclosed AD05876, described in WO2019/051402. Compound 1738179 comprises the sequence and chemistry as described previously for AD05876, with a different GalNAc conjugate. Compound 1738179 described in nomenclature used for compounds provided herein is as follows: the antisense oligonucleotide of Compound 1738179 has a sequence (from 5′ to 3′): UCACUGAGAAUACUGUCCCGU (SEQ ID NO: 9); a sugar motif (from 5′ to 3′): yfyfyfyyyyyfyfyfyfyfy, wherein each wherein each ‘y’ represents a ribo-2′-OMe sugar moiety, each ‘f’ represents a ribo-2′-F sugar moiety; and an internucleoside linkage motif (from 5′ to 3′): sssoooooooooooooooos; wherein each ‘s’ represents a phosphorothioate internucleoside linkage, and each ‘o’ represents a phosphodiester internucleoside linkage. The sense oligonucleotide of Compound 1738179 has the sequence (from 5′ to 3′): ACGGGACAGUAUUCUCAGUIA (SEQ ID NO: 10), wherein ‘I’ represents inosine; the sugar motif (from 5′ to 3′): yyyyyyyyfffyyyyyyyyyy, wherein each wherein each ‘y’ represents a ribo-2′-OMe sugar moiety, each ‘f’ represents a ribo-2′-F sugar moiety; and the internucleoside linkage motif (from 5′ to 3′): oooooooooooooooooooo; wherein each ‘o’ represents a phosphodiester internucleoside linkage. The sense oligonucleotide of Compound No. 1738179 contains an inverted abasic 2′-β-D-deoxyribosyl sugar moiety at the 5′ end and at the 3′ end, connected with a phosphorothioate internucleoside linkage. Compound 1738179 includes a trivalent GalNAc₃ phosphorothioate, THA-GalNAc, conjugated to the 5′ end of the sense oligonucleotide, as described herein for other compounds; and as compared to the previously described AD05876 compound, in which the trivalent GalNAc ‘(NAG37)s’ is conjugated to the 5′ end of the sense oligonucleotide, the structure of which is:

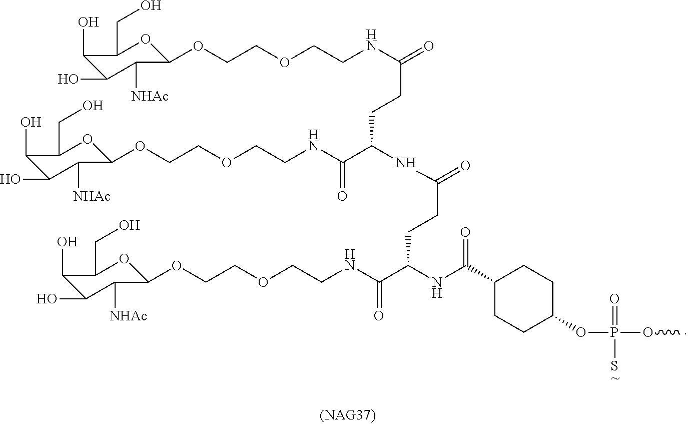

Activity of prepared Compound 1738179 was compared to published literature results for AD05876 and found to be equivalent. See Wong, So. C., et al., National Lipid Association 2019 Scientific Sessions Poster Abstract ID #324, 15 May 2019; and WO2019/051402.

To evaluate the effects of oligomeric duplex compounds on levels of ApoCIII protein and levels of triglycerides (TRIG), blood plasma was collected at various timepoints as indicated, and analyzed using an automated clinical chemistry analyzer (Hitachi Olympus AU400c, Melville, NY). ApoCIII protein levels were detected using an ApoCIII assay kit from Randox (Catalog #LP3865). Results from individual mice were averaged for each group of mice and are presented as percent ApoCIII protein and percent TRIG relative to the amount of ApoCIII protein and amount of TRIG at baseline (i.e., result at timepoint as % baseline for each animal was calculated, then average of the group reflected as % Baseline). “N.C.” refers to values that were not calculated.

TABLE 10
Effect of oligomeric duplex compounds on
ApoCIII protein and triglyceride levels

| | | Plasma | |
|---|---|---|---|
| | | ApoCIII | Plasma |
| Compound | Timepoint | Protein | TRIG |
| No. | (Days) | (% Baseline) | (% Baseline) |
| PBS | 8 | 110 | 107 |
| | 15 | 107 | 93 |
| | 29 | 108 | 86 |
| | 43 | 108 | 80 |
| | 57 | 102 | 86 |
| | 71 | 85 | 78 |
| | 85 | 86 | 81 |
| 1738179 | 8 | 7 | 11 |
| | 15 | 9 | 12 |
| | 29 | 22 | 22 |
| | 43 | 57 | 49 |
| | 57 | 87 | 75 |
| | 71 | 71 | 77 |
| | 85 | 69 | 61 |
| 1692958 | 8 | 8 | 9 |
| | 15 | 6 | 8 |
| | 29 | 5 | 8 |
| | 43 | 13 | 11 |
| | 57 | 23 | 16 |
| | 71 | 24 | 19 |
| | 85 | 30 | 22 |

‡ indicates fewer than 4 samples available

TABLE 11
Effect of oligomeric duplex compounds
on APOC3 and triglyceride levels

| | | APOC3 | |
|---|---|---|---|
| | | Plasma | |
| Compound | Timepoint | Protein | Plasma TRIG |
| No. | (Days) | (% Baseline) | (% Baseline) |
| PBS | 8 | 92 | 78 |
| | 15 | 108 | 94 |
| | 29 | 74 | 73 |
| | 43 | 78 | 68 |
| | 57 | 47 | 73 |
| | 85 | 96 | 71 |
| 1692958 | 8 | 25 | 19 |
| | 15 | 26 | 18 |
| | 29 | 16 | 19 |
| | 43 | 25 | 32 |
| | 57 | 22 | 36 |
| | 85 | 121 | 130 |
| 1692964 | 8 | 17 | 16 |
| | 15 | 26 | 21 |
| | 29 | 24 | 28 |
| | 43 | 29 | 29 |
| | 57 | 16 | 34 |
| | 85 | 60 | 50 |
| 1714226 | 8 | 21 | 17 |
| | 15 | 19 | 16 |
| | 29 | 19 | 19 |
| | 43 | 28 | 29 |
| | 57 | 20 | 36 |
| | 85 | 83 | 63 |
| 1714229 | 8 | 27 | 25 |
| | 15 | 26 | 24 |
| | 29 | 31 | 32 |
| | 43 | 32 | 27 |
| | 57 | 34 | 64 |
| | 85 | N.C. | N.C. |
| 1704071 | 8 | 18 | 9 |
| | 15 | 26 | 24 |
| | 29 | 21 | 29 |
| | 43 | 40 | 42 |
| | 57 | 41 | 71 |
| | 85 | N.C. | N.C. |
| 1714244 | 8 | 36 | 29 |
| | 15 | 28 | 22 |
| | 29 | 36 | 39 |
| | 43 | 41 | 45 |
| | 57 | 36 | 85 |
| | 85 | N.C. | N.C. |
| 1714253 | 8 | 21 | 21 |
| | 15 | 22 | 16 |
| | 29 | 25 | 26 |
| | 43 | 23 | 26 |
| | 57 | 24 | 48 |
| | 85 | 120 | 94 |
| 1714259 | 8 | 38 | 30 |
| | 15 | 32 | 28 |
| | 29 | 35 | 32 |
| | 43 | 44 | 42 |
| | 57 | 34 | 57 |
| | 85 | 141‡ | 136‡ |

‡indicates fewer than 3 samples available

TABLE 12
Effect of oligomeric duplex compounds
on APOC3 and triglyceride levels

| | | APOC3 | |
|---|---|---|---|
| | | Plasma | Plasma |
| Compound | Timepoint | Protein | TRIG |
| No. | (Days) | (% Baseline) | (% Baseline) |
| PBS | 8 | 73 | 128 |
| | 29 | 117 | 100 |
| | 43 | 119 | 111 |
| | 57 | 123 | 117 |
| | 85 | 119 | 109 |
| 1692958 | 8 | 14 | 16 |
| | 29 | 8 | 12 |
| | 43 | 20 | 19 |
| | 57 | 25 | 28 |
| | 85 | 49 | 45 |
| 1704096 | 8 | 65 | 57 |
| | 29 | 37 | 35 |
| | 43 | 50 | 41 |
| | 57 | 56 | 60 |
| | 85 | 81 | 83 |
| 1717167 | 8 | 16 | 13 |
| | 29 | 4 | 10 |
| | 43 | 19 | 10 |
| | 57 | 21 | 21 |
| | 85 | 49 | 37 |
| 1717170 | 8 | 20 | 15 |
| | 29 | 14 | 16 |
| | 43 | 39 | 18 |
| | 57 | 31 | 24 |
| | 85 | 83 | 66 |

‡ indicates fewer than 3 samples available

TABLE 13
Effect of oligomeric duplex compounds
on APOC3 and triglyceride levels

| | | APOC3 | |
|---|---|---|---|
| | | Plasma | Plasma |
| Compound | Timepoint | Protein | TRIG |
| No. | (Days) | (% Baseline) | (% Baseline) |
| PBS | 15 | 87 | 73 |
| | 29 | 90 | 87 |
| | 57 | 90 | 88 |
| | 85 | 76 | 73 |
| 1692958 | 15 | 15 | 12 |
| | 29 | 18 | 15 |
| | 57 | 30 | 22 |
| | 85 | 60 | 45 |
| 1718689 | 15 | 17 | 13 |
| | 29 | 20 | 16 |
| | 57 | 28 | 27 |
| | 85 | 59‡ | 53‡ |
| 1718692 | 15 | 16 | 12 |
| | 29 | 23 | 15 |
| | 57 | 51 | 37 |
| | 85 | 59 | 43 |
| 1722860 | 15 | 2 | 10 |
| | 29 | 9 | 14 |
| | 57 | 28‡ | 26‡ |
| | 85 | 65‡ | 58‡ |

‡indicates fewer than 3 samples available

TABLE 14
Effect of oligomeric duplex compounds
on APOC3 and triglyceride levels

| | | APOC3 | |
|---|---|---|---|
| | | Plasma | |
| Compound | Timepoint | Protein | Plasma TRIG |
| No. | (Days) | (% Baseline) | (% Baseline) |
| PBS | 15 | 77 | 108 |
| | 29 | 83 | 130 |
| | 57 | 81 | 152 |
| | 85 | 74 | 103 |
| 1692958 | 15 | 3 | 7 |
| | 29 | 7 | 9 |
| | 57 | 22 | 23 |
| | 85 | 49 | 52 |
| 1692964 | 15 | 11 | 17 |
| | 29 | 23 | 28 |
| | 57 | 33 | 50 |
| | 85 | 48 | 65 |
| 1723889 | 15 | 11 | 18 |
| | 29 | 23 | 18 |
| | 57 | 29 | 42 |
| | 85 | 49 | 56 |
| 1723892 | 15 | 26 | 30 |
| | 29 | 32 | 55 |
| | 57 | 54 | 73 |
| | 85 | N.C. | N.C. |
| 1723895 | 15 | 27 | 24 |
| | 29 | 27 | 33 |
| | 57 | 40 | 48 |
| | 85 | 49 | 51 |
| 1731577 | 15 | 6 | 8 |
| | 29 | 10 | 11 |
| | 57 | 24 | 25 |
| | 85 | 47 | 46 |

‡ indicates fewer than 3 samples available

TABLE 15
Effect of oligomeric duplex compounds
on APOC3 and triglyceride levels

| | | APOC3 Plasma | |
|---|---|---|---|
| Compound | Timepoint | Protein | Plasma TRIG |
| No. | (Days) | (% Baseline) | (% Baseline) |
| PBS | 11 | 87‡ | 88‡ |
| | 29 | 112‡ | 112‡ |
| | 57 | 93‡ | 131‡ |
| | 85 | 80‡ | 141‡ |
| 1692958 | 11 | 10 | 12 |
| | 29 | 9 | 11 |
| | 57 | 27 | 29 |
| | 85 | 47 | 60 |
| 1692964 | 11 | 17 | 19 |
| | 29 | 19 | 21 |
| | 57 | 34 | 37 |
| | 85 | 54 | 79 |
| 1735455 | 11 | 18 | 22 |
| | 29 | 24 | 20 |
| | 57 | 49 | 62 |
| | 85 | N.C. | N.C. |
| 1735463 | 11 | 38 | 44 |
| | 29 | N.C. | N.C. |
| | 57 | N.C. | N.C. |
| | 85 | N.C. | N.C. |
| 1738342 | 11 | 4 | 7 |
| | 29 | 10 | 8 |
| | 57 | 29 | 21 |
| | 85 | 49 | 57 |
| 1738345 | 11 | 15 | 15 |
| | 29 | 24 | 20 |
| | 57 | 34 | 31 |
| | 85 | 41 | 40 |
| 1738348 | 11 | 16 | 17 |
| | 29 | 34 | 23 |
| | 57 | 57 | 69 |
| | 85 | N.C. | N.C |
| 1738354 | 11 | 12 | 12 |
| | 29 | 20 | 12 |
| | 57 | 25 | 24 |
| | 85 | 32 | 32 |

‡indicates fewer than 3 samples available

TABLE 16
Effect of oligomeric duplex compounds
on APOC3 and triglyceride levels

| | | APOC3 Plasma | |
|---|---|---|---|
| Compound | Timepoint | Protein | Plasma TRIG |
| No. | (Days) | (% Baseline) | (% Baseline) |
| PBS | 15 | 81 | 94 |
| | 29 | 83 | 80 |
| | 57 | 56 | 65 |
| | 85 | 63 | 89 |
| 1692958 | 15 | 6 | 11 |
| | 29 | 11 | 13 |
| | 57 | 23 | 33 |
| | 85 | 34 | 43 |
| 1732457 | 15 | 12 | 16 |
| | 29 | 17 | 17 |
| | 57 | 27 | 27 |
| | 85 | 62 | 87 |
| 1743461 | 15 | 12 | 13 |
| | 29 | 12 | 14 |
| | 57 | 21 | 22 |
| | 85 | 35 | 36 |
| 1743464 | 15 | 12 | 13 |
| | 29 | 13 | 11 |
| | 57 | 18 | 17 |
| | 85 | 33‡ | 33‡ |
| 1743470 | 15 | 13 | 9 |
| | 29 | 14 | 11 |
| | 57 | 29 | 29 |
| | 85 | 40 | 43 |

‡indicates fewer than 3 samples available

TABLE 17
Effect of oligomeric duplex compounds
on APOC3 and triglyceride levels

| | | APOC3 Plasma | |
|---|---|---|---|
| Compound | Timepoint | Protein | Plasma TRIG |
| No. | (Days) | (% Baseline) | (% Baseline) |
| PBS | 15 | 95 | 152 |
| | 29 | 128 | 177 |
| | 57 | 105 | 133 |
| | 85 | 104 | 106 |
| 1692958 | 15 | 9 | 11 |
| | 29 | 10 | 18 |
| | 57 | 31 | 29 |
| | 85 | 52 | 41 |
| 1754076 | 15 | 15 | 22 |
| | 29 | 23 | 29 |
| | 57 | N.C. | N.C. |
| | 85 | N.C. | N.C. |
| 1754466 | 15 | 18 | 28 |
| | 29 | 25 | 40 |
| | 57 | N.C. | N.C. |
| | 85 | N.C. | N.C. |
| 1754787 | 15 | 23 | 22 |
| | 29 | 38 | 36 |
| | 57 | N.C. | N.C. |
| | 85 | N.C. | N.C. |
| 1754958 | 15 | 20 | 23 |
| | 29 | 22 | 21 |
| | 57 | N.C. | N.C. |
| | 85 | N.C. | N.C. |
| 1754961 | 15 | 11 | 11 |
| | 29 | 26 | 26 |
| | 57 | 40 | 34 |
| | 85 | 66 | 55 |
| 1754964 | 15 | 30 | 36 |
| | 29 | 50 | 52 |
| | 57 | N.C. | N.C. |
| | 85 | N.C. | N.C. |
| 1754970 | 15 | 8‡ | 15‡ |
| | 29 | 6‡ | 18‡ |
| | 57 | 43‡ | 44‡ |
| | 85 | 78‡ | 77‡ |
| 1754973 | 15 | 10‡ | 15‡ |
| | 29 | 16‡ | 23‡ |
| | 57 | 43‡ | 42‡ |
| | 85 | 61‡ | 67‡ |
| 1754976 | 15 | 9 | 11 |
| | 29 | 10‡ | 13‡ |
| | 57 | 30‡ | 23‡ |
| | 85 | 37‡ | 19‡ |
| 1755057 | 15 | 12 | 14 |
| | 29 | 19 | 27 |
| | 57 | 50 | 59 |
| | 85 | 80 | 84 |
| 1755066 | 15 | 13 | 16 |
| | 29 | 18 | 26 |
| | 57 | 29 | 39 |
| | 85 | 46 | 48 |
| 1755060 | 15 | 10 | 11 |
| | 29 | 9 | 13 |
| | 57 | 41 | 41 |
| | 85 | 61 | 70 |
| 1755063 | 15 | 4 | 20 |
| | 29 | 6 | 25 |
| | 57 | 24 | 46 |
| | 85 | 41 | 65 |
| 1755069 | 15 | 7 | 15 |
| | 29 | 9 | 18 |
| | 57 | 30 | 40 |
| | 85 | 64 | 104 |
| 1755072 | 15 | 8 | 9 |
| | 29 | 14‡ | 15‡ |
| | 57 | 47‡ | 46‡ |
| | 85 | 78‡ | 75‡ |
| 1755096 | 15 | 38‡ | 53‡ |
| | 29 | 56‡ | 74‡ |
| | 57 | N.C. | N.C. |
| | 85 | N.C. | N.C. |
| 1755099 | 15 | 34 | 44 |
| | 29 | 58 | 102 |
| | 57 | N.C. | N.C. |
| | 85 | N.C. | N.C. |

‡indicates fewer than 3 samples available

TABLE 18
Effect of oligomeric duplex compounds
on APOC3 and triglyceride levels

| | | APOC3 Plasma | |
|---|---|---|---|
| Compound | Timepoint | Protein | Plasma TRIG |
| No. | (Days) | (% Baseline) | (% Baseline) |
| PBS | 15 | 112 | 108 |
| | 29 | 110 | 115 |
| | 57 | 118 | 111 |
| | 85 | 126 | 115 |
| 1692958 | 15 | 5 | 8 |
| | 29 | 7 | 6 |
| | 57 | 20 | 16 |
| | 85 | 44 | 27 |
| 1735455 | 15 | 10 | 8 |
| | 29 | 21 | 17 |
| | 57 | 22 | 15 |
| | 85 | 35‡ | 19‡ |
| 1757505 | 15 | 14 | 10 |
| | 29 | 20‡ | 14‡ |
| | 57 | 32‡ | 21‡ |
| | 85 | 66‡ | 36‡ |
| 1757508 | 15 | 2 | 4 |
| | 29 | 9 | 7 |
| | 57 | 19‡ | 15‡ |
| | 85 | 37‡ | 25‡ |

‡indicates fewer than 3 samples available

TABLE 19
Effect of oligomeric duplex compounds
on APOC3 and triglyceride levels

| | | APOC3 Plasma | |
|---|---|---|---|
| Compound | Timepoint | Protein | Plasma TRIG |
| No. | (Days) | (% Baseline) | (% Baseline) |
| PBS | 15 | 91 | 74 |
| | 29 | 93 | 71 |
| | 57 | 86 | 54 |
| | 85 | 69 | 53 |
| 1692958 | 15 | 5‡ | 8‡ |
| | 29 | 5‡ | 7‡ |
| | 57 | 13‡ | 11‡ |
| | 85 | 48‡ | 31‡ |
| 1744810 | 15 | 8 | 7 |
| | 29 | 14 | 9 |
| | 57 | 24 | 12 |
| | 85 | 55 | 29 |
| 1757505 | 15 | 12‡ | 9‡ |
| | 29 | 18‡ | 11‡ |
| | 57 | 28‡ | 17‡ |
| | 85 | 48‡ | 25‡ |
| 1758193 | 15 | 7 | 8 |
| | 29 | 8 | 8 |
| | 57 | 25 | 16 |
| | 85 | 44 | 24 |
| 1758231 | 15 | 5 | 7 |
| | 29 | 8 | 7 |
| | 57 | 17 | 10 |
| | 85 | 39 | 22 |

‡indicates fewer than 3 samples available

TABLE 20
Effect of oligomeric duplex compounds
on APOC3 and triglyceride levels

| | | ApoCIII | |
|---|---|---|---|
| Compound | Timepoint | Protein | TRIG |
| No. | (Days) | (% Baseline) | (% Baseline) |
| PBS | 15 | 106 | 83 |
| | 36 | 112 | 100 |
| | 57 | 97 | 89 |
| | 85 | 106 | 119 |
| 1738179 | 15 | 22 | 10 |
| | 36 | 71 | 54 |
| | 57 | 78‡ | 65‡ |
| | 85 | 87‡ | 71‡ |
| 1758231 | 15 | 10 | 6 |
| | 36 | 22 | 13 |
| | 57 | 25 | 20 |
| | 85 | 37 | 29 |

‡indicates fewer than 4 samples available

TABLE 21
Effect of oligomeric duplex compounds
on APOC3 and triglyceride levels

| | | APOC3 Plasma | |
|---|---|---|---|
| Compound | Timepoint | Protein | Plasma TRIG |
| No. | (Days) | (% Baseline) | (% Baseline) |
| PBS | 7 | 110 | 107 |
| | 14 | 107 | 93 |
| | 28 | 108 | 86 |
| | 42 | 108 | 80 |
| | 56 | 102 | 86 |
| | 70 | 85 | 78 |
| | 84 | 86 | 81 |
| | 105 | 70 | 63 |
| | 154 | 66 | 40 |
| 1692981 | 7 | 10 | 12 |
| | 14 | 6 | 8 |
| | 28 | 8 | 10 |
| | 42 | 20‡ | 21‡ |
| | 56 | 33‡ | 25‡ |
| | 70 | 40‡ | 36‡ |
| | 84 | 58‡ | 48‡ |
| | 105 | 46‡ | 52‡ |
| | 154 | 75‡ | 71‡ |
| 1692984 | 7 | 19 | 24 |
| | 14 | 8 | 9 |
| | 28 | 9 | 12 |
| | 42 | 19 | 21 |
| | 56 | 31‡ | 29‡ |
| | 70 | 37‡ | 39‡ |
| | 84 | 53‡ | 47‡ |
| | 105 | 45‡ | 56‡ |
| | 154 | 52‡ | 46‡ |
| 1706916 | 7 | 15 | 15 |
| | 14 | 8 | 9 |
| | 28 | 8 | 11 |
| | 42 | 17 | 18 |
| | 56 | 34 | 29 |
| | 70 | 42 | 34 |
| | 84 | 56 | 37 |
| | 105 | 42 | 43 |
| | 154 | 63 | 49 |
| 1738179 | 7 | 7 | 10 |
| | 14 | 9 | 12 |
| | 28 | 22 | 21 |
| | 42 | 57 | 49 |
| | 56 | 87 | 75 |
| | 70 | 71 | 77 |
| | 84 | 68 | 61 |
| | 105 | 58 | 61 |
| | 154 | 55 | 39 |
| 1692972 | 7 | 20 | 20 |
| | 14 | 16 | 18 |
| | 28 | 21‡ | 21‡ |
| | 42 | 41‡ | 38‡ |
| | 56 | 48‡ | 46‡ |
| | 70 | 54‡ | 57‡ |
| | 84 | 50‡ | 36‡ |
| | 105 | 54‡ | 55‡ |
| | 154 | 60‡ | 63‡ |

‡indicates fewer than 4 samples available

TABLE 22
Effect of oligomeric duplex compounds
on APOC3 and triglyceride levels

| | | APOC3 Plasma | |
|---|---|---|---|
| Compound | Timepoint | Protein | Plasma TRIG |
| No. | (Days) | (% Baseline) | (% Baseline) |
| PBS | 14 | 87 | 73 |
| | 28 | 90 | 87 |
| | 56 | 90 | 88 |
| | 84 | 76 | 73 |
| 1718718 | 14 | 41 | 32 |
| | 28 | 41 | 38 |
| | 56 | 55 | 51 |
| | 84 | 54 | 54 |
| 1718721 | 14 | 29 | 22 |
| | 28 | 38 | 30 |
| | 56 | 53 | 39 |
| | 84 | 67 | 48 |
| 1718728 | 14 | 43 | 44 |
| | 28 | 48 | 53 |
| | 56 | N.C. | N.C. |
| | 84 | N.C. | N.C. |
| 1722860 | 14 | 2 | 10 |
| | 28 | 9 | 14 |
| | 56 | 28‡ | 26‡ |
| | 84 | 65‡ | 57‡ |

‡indicates fewer than 3 samples available

TABLE 23
Effect of oligomeric duplex compounds
on APOC3 and triglyceride levels

| | | APOC3 Plasma | |
|---|---|---|---|
| Compound | Timepoint | Protein | Plasma TRIG |
| No. | (Days) | (% Baseline) | (% Baseline) |
| PBS | 7 | 92 | 78 |
| | 14 | 108 | 94 |
| | 28 | 74 | 73 |
| | 42 | 78 | 68 |
| | 56 | 47 | 73 |
| 1714256 | 7 | 22 | 15 |
| | 14 | 23 | 14 |
| | 28 | 29 | 25 |
| | 42 | 37 | 36 |
| | 56 | 31 | 51 |
| 1714262 | 7 | 24 | 19 |
| | 14 | 32 | 17 |
| | 28 | 26 | 28 |
| | 42 | 25 | 19 |
| | 56 | 16 | 29 |
| 1714265 | 7 | 47 | 44 |
| | 14 | 42 | 39 |
| | 28 | 61 | 80 |
| | 42 | 82 | 102 |
| | 56 | N.C. | N.C. |

TABLE 24
Effect of oligomeric duplex compounds
on APOC3 and triglyceride levels

| | | APOC3 Plasma | |
|---|---|---|---|
| Compound | Timepoint | Protein | Plasma TRIG |
| No. | (Days) | (% Baseline) | (% Baseline) |
| PBS | 14 | 77 | 108 |
| | 28 | 83 | 130 |
| 1731581 | 14 | 56 | 74 |
| | 28 | 51 | 69 |

TABLE 25
Effect of oligomeric duplex compounds
on APOC3 and triglyceride levels

| | | APOC3 Plasma | |
|---|---|---|---|
| Compound | Timepoint | Protein | Plasma TRIG |
| No. | (Days) | (% Baseline) | (% Baseline) |
| PBS | 13 | 105 | 91 |
| 1743442 | 13 | 21 | 20 |
| 1743450 | 13 | 83 | 31 |
| 1743453 | 13 | 116 | 96 |

Each of the provided oligomeric duplex compounds showed a reduction in plasma ApoCIII protein as well as serum triglycerides as compared to control treatment. Many provided compounds sustain reduction in ApoCIII and triglycerides for longer duration than compound 1738179, a surrogate for prior known APOC3 inhibitor AD05876.

### Example 3: Effect of Oligomeric Duplexes Targeting Human APOC3 in APOC3 Transgenic Mice

APOC3 transgenic mice (described above in Ex. 2) were used to determine effects of various doses of the oligomeric duplex compounds on human APOC3. APOC3 transgenic mice were divided into groups of 3 mice each. Each mouse received a single subcutaneous injection of oligomeric duplex compound at doses indicated. One group of 3 mice received a single subcutaneous injection of PBS and served as a control group.

To evaluate the effect of oligomeric duplex compounds on levels of APOC3 and levels of triglycerides (TRIG), blood plasma was collected at various timepoints as indicated and analyzed using an automated clinical chemistry analyzer (Hitachi Olympus AU400c, Melville, NY). ApoCIII protein levels were detected using either an APOC3 assay kit from Randox (Catalog #LP3865) or an APOC3 kit from Kamiya Biomedical (KAI-006). Results from individual mice were averaged for each group of mice and are presented as percent ApoCIII protein and percent TRIG relative to the amount of ApoCIII protein and amount of TRIG at baseline (i.e., result at timepoint as % baseline for each animal was calculated, then average of the group reflected as % Baseline). “N.C.” refers to values that were not calculated.

TABLE 26
Effect of oligomeric duplex compounds
on APOC3 and triglyceride levels

| | | | ApoCIII | |
|---|---|---|---|---|
| Compound | Dose | Timepoint | Protein | TRIG |
| No. | (mg/kg) | (Days) | (% Baseline) | (% Baseline) |
| PBS | — | 8 | 80 | 87 |
| | | 15 | 84 | 91 |
| | | 22 | 88 | 103 |
| | | 29 | 80 | 91 |
| | | 43 | 82 | 87 |
| | | 57 | 77 | 85 |
| | | 71 | 90 | 98 |
| | | 99 | 70 | 86 |
| | | 120 | 66 | 81 |
| 1692958 | 0.11 | 8 | 63 | 61 |
| | | 15 | 48 | 39 |
| | | 22 | 48 | 58 |
| | | 29 | 67 | 54 |
| | | 43 | N.C. | N.C. |
| | | 57 | N.C. | N.C. |
| | | 71 | N.C. | N.C. |
| | | 99 | N.C. | N.C. |
| | | 120 | N.C. | N.C. |
| | 0.33 | 8 | 32 | 32 |
| | | 15 | 22 | 17 |
| | | 22 | 35 | 35 |
| | | 29 | 37 | 35 |
| | | 43 | 44 | 43 |
| | | 57 | 75 | 75 |
| | | 71 | 96 | 100 |
| | | 99 | N.C. | N.C. |
| | | 120 | N.C. | N.C. |
| | 1 | 8 | 10 | 9 |
| | | 15 | 9 | 11 |
| | | 22 | 12 | 13 |
| | | 29 | 15 | 12 |
| | | 43 | 18 | 14 |
| | | 57 | 21 | 17 |
| | | 71 | 30 | 22 |
| | | 99 | 42 | 45 |
| | | 120 | 42 | 44 |
| | 9 | 8 | 3 | 6 |
| | | 15 | 2 | 3 |
| | | 22 | 2 | 5 |
| | | 29 | 3 | 5 |
| | | 43 | 3 | 4 |
| | | 57 | 5 | 7 |
| | | 71 | 10 | 11 |
| | | 99 | 16 | 16 |
| | | 120 | 16 | 16 |
| 1692964 | 0.11 | 8 | 77 | 63 |
| | | 15 | 67 | 52 |
| | | 22 | 51 | 48 |
| | | 29 | 80 | 59 |
| | | 43 | N.C. | N.C. |
| | | 57 | N.C. | N.C. |
| | | 71 | N.C. | N.C. |
| | | 99 | N.C. | N.C. |
| | | 120 | N.C. | N.C. |
| | 0.33 | 8 | 47 | 54 |
| | | 15 | 36 | 40 |
| | | 22 | 43 | 51 |
| | | 29 | 53 | 60 |
| | | 43 | 61 | 72 |
| | | 57 | 67 | 77 |
| | | 71 | 90 | 132 |
| | | 99 | N.C. | N.C. |
| | | 120 | N.C. | N.C. |
| | 1 | 8 | 23 | 24 |
| | | 15 | 13 | 13 |
| | | 22 | 14 | 16 |
| | | 29 | 17 | 15 |
| | | 43 | 25 | 16 |
| | | 57 | 23 | 19 |
| | | 71 | 27 | 22 |
| | | 99 | 25 | 22 |
| | | 120 | 34 | 38 |
| 1717167 | 0.11 | 8 | 86 | 96 |
| | | 15 | 74 | 85 |
| | | 22 | 74 | 75 |
| | | 29 | 98 | 91 |
| | | 43 | N.C. | N.C. |
| | | 57 | N.C. | N.C. |
| | | 71 | N.C. | N.C. |
| | | 99 | N.C. | N.C. |
| | | 120 | N.C. | N.C. |
| | 0.33 | 8 | 36 | 33 |
| | | 15 | 28 | 28 |
| | | 22 | 37 | 37 |
| | | 29 | 41 | 37 |
| | | 43 | 53 | 47 |
| | | 57 | 62 | 60 |
| | | 71 | 85 | 80 |
| | | 99 | N.C. | N.C. |
| | | 120 | N.C. | N.C. |
| | 1 | 8 | 20 | 24 |
| | | 15 | 9 | 9 |
| | | 22 | 15 | 14 |
| | | 29 | 13 | 14 |
| | | 43 | 24 | 21 |
| | | 57 | 29 | 27 |
| | | 71 | 38 | 40 |
| | | 99 | 45 | 55 |
| | | 120 | 55 | 72 |
| 1717170 | 0.11 | 8 | 88 | 103 |
| | | 15 | 89 | 89 |
| | | 22 | 85 | 83 |
| | | 29 | 104 | 107 |
| | | 43 | N.C. | N.C. |
| | | 57 | N.C. | N.C. |
| | | 71 | N.C. | N.C. |
| | | 99 | N.C. | N.C. |
| | | 120 | N.C. | N.C. |
| | 0.33 | 8 | 62 | 62 |
| | | 15 | 46 | 37 |
| | | 22 | 50 | 57 |
| | | 29 | 59 | 48 |
| | | 43 | 66 | 67 |
| | | 57 | 75 | 85 |
| | | 71 | 91 | 108 |
| | | 99 | N.C. | N.C. |
| | | 120 | N.C. | N.C. |
| | 1 | 8 | 13 | 13 |
| | | 15 | 9 | 10 |
| | | 22 | 8 | 12 |
| | | 29 | 14 | 13 |
| | | 43 | 22 | 18 |
| | | 57 | 30 | 28 |
| | | 71 | 35 | 36 |
| | | 99 | 47 | 50 |
| | | 120 | 54 | 68 |
| 1722860 | 0.11 | 8 | 81 | 78 |
| | | 15 | 66 | 59 |
| | | 22 | 88 | 91 |
| | | 29 | 90 | 102 |
| | | 43 | N.C. | N.C. |
| | | 57 | N.C. | N.C. |
| | | 71 | N.C. | N.C. |
| | | 99 | N.C. | N.C. |
| | | 120 | N.C. | N.C. |
| | 0.33 | 8 | 28 | 25 |
| | | 15 | 24 | 18 |
| | | 22 | 32 | 33 |
| | | 29 | 35 | 30 |
| | | 43 | 53 | 51 |
| | | 57 | 53 | 44 |
| | | 71 | 79 | 88 |
| | | 99 | N.C. | N.C. |
| | | 120 | N.C. | N.C. |
| | 1 | 8 | 13 | 14 |
| | | 15 | 8 | 9 |
| | | 22 | 16 | 13 |
| | | 29 | 13 | 12 |
| | | 43 | 19 | 12 |
| | | 57 | 20 | 20 |
| | | 71 | 33 | 38 |
| | | 99 | 46 | 54 |
| | | 120 | 59 | 81 |
| 1731577 | 0.11 | 8 | 71 | 71 |
| | | 15 | 67 | 70 |
| | | 22 | 79 | 92 |
| | | 29 | 80 | 78 |
| | | 43 | N.C. | N.C. |
| | | 57 | N.C. | N.C. |
| | | 71 | N.C. | N.C. |
| | | 99 | N.C. | N.C. |
| | | 120 | N.C. | N.C. |
| | 0.33 | 8 | 41 | 38 |
| | | 15 | 35 | 34 |
| | | 22 | 43 | 50 |
| | | 29 | 60 | 53 |
| | | 43 | 65 | 69 |
| | | 57 | 85 | 95 |
| | | 71 | 103 | 139 |
| | | 99 | N.C. | N.C. |
| | | 120 | N.C. | N.C. |
| | 1 | 8 | 15 | 17 |
| | | 15 | 13 | 12 |
| | | 22 | 22 | 13 |
| | | 29 | 19 | 13 |
| | | 43 | 31 | 28 |
| | | 57 | 41 | 43 |
| | | 71 | 54 | 53 |
| | | 99 | 71 | 97 |
| | | 120 | 67 | 85 |

‡ indicates fewer than 3 samples available

TABLE 27
Effect of oligomeric duplex compounds
on ApoCIII and triglyceride levels

| | | | ApoCIII | |
|---|---|---|---|---|
| Compound | Dose | Timepoint | Protein | TRIG |
| No. | (mg/kg) | (Days) | (% Baseline) | (% Baseline) |
| PBS | | 8 | 162 | 137 |
| | | 15 | 134 | 112 |
| | | 29 | 115 | 122 |
| | | 64 | 77 | 72 |
| | | 85 | 85 | 88 |
| 1692958 | 1 | 8 | 17 | 14 |
| | | 15 | 15 | 14 |
| | | 29 | 19 | 20 |
| | | 64 | 32 | 33 |
| | | 85 | 46 | 42 |
| | 2 | 8 | 12 | 9 |
| | | 15 | 10 | 9 |
| | | 29 | 8 | 10 |
| | | 64 | 21 | 23 |
| | | 85 | 28 | 28 |
| | 3 | 8 | 7 | 8 |
| | | 15 | 9 | 8 |
| | | 29 | 6 | 7 |
| | | 64 | 18 | 20 |
| | | 85 | 28 | 25 |
| | 9 | 8 | 4 | 3 |
| | | 15 | 5 | 4 |
| | | 29 | 3 | 5 |
| | | 64 | 10 | 11 |
| | | 85 | 20 | 15 |
| 1735455 | 1 | 8 | 25 | 19 |
| | | 15 | 20 | 16 |
| | | 29 | 32 | 17 |
| | | 64 | 52‡ | 55‡ |
| | | 85 | 64‡ | 64‡ |
| | 2 | 8 | 9 | 9 |
| | | 15 | 11 | 11 |
| | | 29 | 18 | 15 |
| | | 64 | 58 | 66 |
| | | 85 | 72 | 67 |
| | 3 | 8 | 10 | 8 |
| | | 15 | 10 | 8 |
| | | 29 | 18 | 14 |
| | | 64 | 38 | 32 |
| | | 85 | 42 | 32 |
| 1738342 | 1 | 8 | 17 | 14 |
| | | 15 | 15 | 12 |
| | | 29 | 22 | 16 |
| | | 64 | 39 | 29 |
| | | 85 | 66 | 58 |
| | 2 | 8 | 10 | 9 |
| | | 15 | 13 | 10 |
| | | 29 | 10 | 11 |
| | | 64 | 26 | 25 |
| | | 85 | 37 | 29 |
| | 3 | 8 | 6 | 4 |
| | | 15 | 6 | 4 |
| | | 29 | 6 | 5 |
| | | 64 | 17 | 11 |
| | | 85 | 29 | 20 |
| 1743461 | 1 | 8 | 14 | 10 |
| | | 15 | 14 | 12 |
| | | 29 | 18 | 16 |
| | | 64 | 44 | 46 |
| | | 85 | 63 | 62 |
| | 2 | 8 | 5 | 6 |
| | | 15 | 9 | 7 |
| | | 29 | 5 | 6 |
| | | 64 | 17 | 14 |
| | | 85 | 29 | 22 |
| | 3 | 8 | 4 | 6 |
| | | 15 | 6‡ | 4‡ |
| | | 29 | 6‡ | 10‡ |
| | | 64 | 22‡ | 22‡ |
| | | 85 | 46‡ | 55‡ |
| 1743470 | 1 | 8 | 18 | 13 |
| | | 15 | 8 | 7 |
| | | 29 | 16 | 13 |
| | | 64 | 32 | 33 |
| | | 85 | 43 | 38 |
| | 2 | 8 | 13 | 11 |
| | | 15 | 11 | 11 |
| | | 29 | 10 | 10 |
| | | 64 | 25 | 27 |
| | | 85 | 39 | 39 |
| | 3 | 8 | 6 | 4 |
| | | 15 | 5 | 7 |
| | | 29 | 7 | 8 |
| | | 64 | 16‡ | 13‡ |
| | | 85 | 42‡ | 45‡ |

‡indicates fewer than 3 samples available

Each of the oligomeric duplex compounds tested demonstrated dose dependent reduction in plasma ApoCIII protein as well as serum triglycerides as compared to control treatment. Many compounds sustain reduction in ApoCIII protein and triglycerides to 85 days.

### Example 4: Effect of Oligomeric Duplexes Targeting Human APOC3 in APOC3 Transgenic Mice

APOC3 transgenic mice (described in Ex 2 above) were used to determine effects of the oligomeric duplex compounds on human APOC3. APOC3 transgenic mice were divided into groups of 3 mice each. Each mouse received a single subcutaneous injection of oligomeric duplex compound at various doses as indicated. One group of 4 mice received a single subcutaneous injection of PBS, served as a control group.

One week post treatment mice were sacrificed. To evaluate the effect of oligomeric duplex compounds on levels of APOC3 and levels of triglycerides (TRIG), blood plasma was collected and analyzed using an automated clinical chemistry analyzer (Hitachi Olympus AU400c, Melville, NY). ApoCIII protein levels were analyzed either with an ApoCIII assay kit from Randox (Catalog #LP3865) or with an ApoCIII kit from Kamiya Biomedical (Catalog #KAI-006). Results from individual mice were averaged for each group of mice and are presented as percent ApoCIII protein and percent TRIG relative to the amount of APOC3 and amount of TRIG at baseline (i.e., result at timepoint as % baseline for each animal was calculated, then average of the group reflected as % Baseline). The half maximal effective dose (ED₅₀) of each oligomeric duplex was calculated using GraphPad Prism 9 software (GraphPad Software, San Diego, CA) and is presented in μg/kg.

RNA was also extracted from mouse liver of animals for real-time RTPCR analysis of APOC3 RNA expression. Human APOC3 primer probe set RTS1392 (forward sequence TCAGCTTCATGCAGGGTTACAT (SEQ ID NO: 90) reverse sequence ACGCTGCTCAGTGCATCCT (SEQ ID NO: 91); probe sequence AAGCACGCCACCAAGACCGCC, (SEQ ID NO: 92) was used to measure human APOC3 RNA levels. APOC3 RNA levels were normalized to total RNA content, as measured by RIBOGREEN®. Results are presented as percent APOC3 RNA, relative to the amount of APOC3 RNA in PBS treated animals (% control).

TABLE 28
Effect of oligomeric duplex compounds on human APOC3

| | | Plasma | | Liver |
|---|---|---|---|---|
| | | ApoCIII | Plasma | APOC3 |
| Compound | Dose | Protein | TRIG | RNA |
| No. | (mg/kg) | (% baseline) | (% baseline) | (% control) |
| 1692958 | 0.03 | 5 | 15 | 27 |
| | 0.1 | 2 | 7 | 16 |
| | 0.3 | 1 | 3 | 16 |
| | 0.9 | 1 | 6 | 9 |
| 1755069 | 0.004 | 56 | 70 | 75 |
| | 0.012 | 31 | 35 | 52 |
| | 0.035 | 23 | 29 | 45 |
| | 0.106 | 3 | 7 | 30 |
| 1755072 | 0.005 | 32 | 32 | 60 |
| | 0.015 | 9 | 12 | 34 |
| | 0.046 | 11‡ | 11‡ | 38‡ |
| | 0.139 | 2 | 7 | 19 |
| 1758231 | 0.006 | 27 | 29 | 46 |
| | 0.018 | 9 | 12 | 32 |
| | 0.055 | 9 | 10 | 37 |
| | 0.166 | 3 | 6 | 21 |

‡indicates fewer than 3 samples available

Each of the oligomeric duplex compounds tested suggested dose dependent reduction in plasma ApoCIII protein as well as triglycerides as compared to control treatment. Re-evaluation of this experiment in light of additional studies that showed consistent results across replicates identified discrepancies in doses resulting in reduced confidence in its results. Nevertheless, the trend of dose dependent reduction in ApoCIII protein and reduction in triglycerides was consistent with a number of other experiments carried out in replicates and described herein.

### Example 5: Effect of Oligomeric Duplexes Targeting Human APOC3 in APOC3 Transgenic Mice

APOC3 transgenic mice (described above in Ex. 2) were used to determine effects of the oligomeric duplex compounds on human APOC3. APOC3 transgenic mice were divided into groups of 3 mice each. Each mouse received a single subcutaneous injection of oligomeric duplex compound at doses indicated. One group of 4 mice received a single subcutaneous injection of PBS, served as a control group.

Two weeks post treatment mice were sacrificed. To evaluate the effect of oligomeric duplex compounds on levels of APOC3 and on levels of triglycerides (TRIG), blood plasma was collected and analyzed using an automated clinical chemistry analyzer (Hitachi Olympus AU400c, Melville, NY). ApoCIII protein levels were analyzed with an ApoCIII assay kit from Randox (Catalog #LP3865) or an APOC3 kit from Kamiya Biomedical (Catalog #KAI-006). Results from individual mice were averaged for each group of mice and are presented as percent APOC3 and percent TRIG relative to the amount of ApoCIII and amount of TRIG at baseline (i.e., result at timepoint as % baseline for each animal was calculated, then average of the group reflected as % Baseline).

RNA was also extracted from mouse liver for real-time RTPCR analysis of APOC3 RNA expression. Human APOC3 primer probe set RTS1392 (described above in Ex. 4) was used to measure human APOC3 RNA levels. APOC3 RNA levels were normalized to total RNA content, as measured by RIBOGREEN®. Results are presented as percent APOC3 RNA, relative to the amount of APOC3 in PBS treated animals (% control).

TABLE 29
Effect of oligomeric duplex compounds on human APOC3

| | | ApoCIII Protein | | TRIG | | APOC3 RNA | |
|---|---|---|---|---|---|---|---|
| | | (% Baseline) | | (% Baseline) | | (% control) | |
| Compound | Dose | | ED50 | | ED50 | | ED₅₀ |
| No. | (mg/kg) | Plasma | (mg/kg) | Plasma | (mg/kg) | Liver | (mg/kg) |
| 1738179 | 0.01 | 83 | 0.35 | 122 | 0.35 | 99 | 1.1 |
| | 0.03 | 110 | | 136 | | 100 | |
| | 0.1 | 81‡ | | 102‡ | | 95‡ | |
| | 0.3 | 54 | | 54 | | 113 | |
| | 1.0 | 22 | | 17 | | 71 | |
| 1692958 | 0.1 | 37 | 0.06 | 49 | 0.10 | 74 | 0.28 |
| | 0.3 | 18 | | 24 | | 55 | |
| | 1.0 | 4 | | 8 | | 32 | |
| 1755072 | 0.01 | 86 | 0.07 | 101 | 0.09 | 97 | 0.33 |
| | 0.03 | 58 | | 65 | | 92 | |
| | 0.1 | 51 | | 51 | | 86 | |
| | 0.3 | 20 | | 20 | | 57 | |
| | 1.0 | 3 | | 4 | | 27 | |
| 1758231 | 0.01 | 87 | 0.12 | 102 | 0.15 | 75 | 0.17 |
| | 0.03 | 96 | | 125 | | 82 | |
| | 0.1 | 59 | | 64 | | 75 | |
| | 0.3 | 18 | | 20 | | 46 | |
| | 1.0 | 4 | | 6 | | 19 | |

‡indicates fewer than 3 samples available

Each of the oligomeric duplex compounds tested demonstrated dose dependent reduction in plasma ApoCIII protein as well as triglycerides as compared to control treatment. Dose dependent reduction in liver APOC3 RNA was also shown, and ED50 of compounds were similar across evaluation methods.

### Example 6: Dose-Dependent Inhibition of Human APOC3 RNA in Primary Human Hepatocytes

Oligomeric duplex compounds targeted to a human APOC3 RNA selected from the examples above were tested for their effects on APOC3 RNA in vitro. Primary human hepatocytes (BioIVT, #M0095-P, Male Cryopreserved, Lot ZFW, 95% viability) plated at a density of 30,000 cells per well were treated with oligomeric duplex compound by free uptake at a range of concentrations. After a treatment period of approximately 96 hours at indicated concentration, total RNA was isolated from the cells and APOC3 RNA levels were measured by quantitative real-time RT-PCR. Human APOC3 primer-probe set RTS1392 (described in Ex. 4) was used to measure RNA levels as described above. APOC3 RNA levels were normalized to human GADPH, which was amplified using human primer probe set RTS104 (forward sequence GAAGGTGAAGGTCGGAGTC (SEQ ID NO: 93); reverse sequence GAAGATGGTGATGGGATTTC (SEQ ID NO: 94); probe sequence CAAGCTTCCCGTTCTCAGCC (SEQ ID NO: 95). Resulting reduction of APOC3 RNA is presented as percent APOC3 RNA, relative to the amount of APOC3 RNA in untreated control cells (% UTC). The half maximal inhibitory concentration (IC₅₀) of each oligomeric duplex compound was calculated using GraphPad Prism 9 software (GraphPad Software, San Diego, CA). Results are presented in Table 30.

TABLE 30
Effect of oligomeric duplex compound on
APOC3 RNA in primary human hepatocytes

| | APOC3 RNA (% UTC) | | | | | | | | | | |
|---|---|---|---|---|---|---|---|---|---|---|---|
| Compound | 5000 | 1000 | 200 | 40 | 8 | 1.6 | 0.32 | 0.064 | 0.0128 | 0.00256 | IC₅₀ |
| No. | nM | nM | nM | nM | nM | nM | nM | nM | nM | nM | (pM) |
| 1754976 | 5 | 6 | 5 | 7 | 10 | 16 | 32 | 45 | 100 | 101 | 82 |
| 1758231 | 6 | 6 | 7 | 8 | 10 | 13 | 20 | 35 | 61 | 106 | 28 |
| 1755069 | 5 | 8 | 10 | 13 | 17 | 23 | 43 | 74 | 87 | 91 | 214 |
| 1744810 | 6 | 7 | 13 | 16 | 18 | 25 | 46 | 78 | 75 | 100 | 242 |
| 1755063 | 5 | 7 | 8 | 11 | 11 | 15 | 33 | 66 | 83 | 98 | 119 |
| 1757505 | 5 | 9 | 11 | 15 | 18 | 23 | 40 | 76 | 106 | 81 | 235 |
| 1757508 | 4 | 6 | 7 | 10 | 14 | 21 | 40 | 60 | 85 | 118 | 147 |
| 1758193 | 5 | 6 | 7 | 8 | 8 | 13 | 23 | 44 | 66 | 93 | 38 |
| 1692958 | 5 | 5 | 8 | 10 | 12 | 12 | 22 | 39 | 60 | 83 | 25 |
| 1738179 | 6 | 7 | 10 | 10 | 13 | 15 | 27 | 59 | 116 | 115 | 107 |

Each of the oligomeric duplex compounds tested demonstrated dose dependent reduction in cell ApoC3 RNA as compared to untreated control.

### Example 7: Dose-Dependent Inhibition of Human APOC3 RNA in Primary Cynomolgus Hepatocytes

Oligomeric duplex compounds targeted to a human APOC3 RNA selected from the examples above were tested for their effects on APOC3 RNA in vitro. Primary cynomolgus hepatocytes (Sekisui, #PPCH2000, Lot 2110098) cultured in William's E media were plated at a density of 200,000 cells per well and treated with oligomeric duplex compound by free uptake at a range of concentrations. After a treatment period of approximately 72 hours, at indicated concentration, total RNA was isolated from cells and APOC3 RNA levels were measured by quantitative real-time RT-PCR. Human APOC3 primer-probe set RTS1392 (described in Ex. 4) was used to measure RNA levels as described above. APOC3 RNA levels were normalized to total RNA content, as measured by RIBOGREEN®. Reduction of APOC3 RNA is presented as percent APOC3 RNA, relative to the amount of APOC3 RNA in untreated control cells (% UTC). The half maximal inhibitory concentration (IC₅₀) of each oligomeric duplex compound was calculated using GraphPad Prism 9 software (GraphPad Software, San Diego, CA). Results are presented in Table 31.

TABLE 31
Effect of oligomeric duplex compound on APOC3
RNA in primary cynomolgus hepatocytes

| | APOC3 RNA (% UTC) | | | | | | | | | | |
|---|---|---|---|---|---|---|---|---|---|---|---|
| Compound | 5000 | 1000 | 200 | 40 | 8 | 1.6 | 0.32 | 0.064 | 0.0128 | 0.00256 | IC₅₀ |
| No. | nM | nM | nM | nM | nM | nM | nM | nM | nM | nM | (nM) |
| 1754976 | 9 | 10 | 14 | 22 | 48 | 87 | 113 | 114 | 117 | 126 | 9.87 |
| 1758231 | 10 | 13 | 14 | 21 | 43 | 66 | 117 | 130 | 120 | 129 | 6.71 |
| 1755069 | 13 | 14 | 16 | 25 | 44 | 95 | 114 | 106 | 116 | 102 | 10.35 |
| 1744810 | 9 | 11 | 17 | 23 | 45 | 89 | 106 | 114 | 120 | 124 | 9.59 |
| 1755063 | 9 | 7 | 10 | 15 | 33 | 58 | 110 | 106 | 103 | 112 | 3.72 |
| 1757505 | 8 | 9 | 8 | 22 | 38 | 62 | 90 | 124 | 101 | 99 | 4.70 |
| 1692958 | 8 | 7 | 8 | 12 | 24 | 55 | 81 | 91 | 94 | 89 | 2.03 |
| 1755072 | 9 | 12 | 16 | 24 | 58 | 102 | 96 | 109 | 100 | 96 | 13.97 |
| 1757508 | 7 | 8 | 9 | 15 | 28 | 62 | 88 | 98 | 93 | 91 | 3.07 |
| 1758193 | 9 | 10 | 11 | 15 | 33 | 73 | 100 | 90 | 98 | 90 | 4.80 |
| 1738179 | 9 | 8 | 11 | 20 | 30 | 87 | 104 | 110 | 116 | 97 | 5.35 |

Each of the oligomeric duplex compounds tested demonstrated dose dependent reduction in cell ApoC3 RNA as compared to untreated control.

### Example 8: Selectivity of Oligomeric Duplexes Targeted to APOC3 in Primary Human Hepatocytes

Off-target effects of oligomeric duplexes that target a human APOC3 RNA selected from the examples above were tested in primary human hepatocytes. Primary human hepatocytes (BioIVT, #M0095-P, Male Cryopreserved, Lot ZFW, 95% viability) plated at a density of 30,000 cells per well were treated with oligomeric duplex compound selected from U.S. Pat. Nos. 1,754,976, 1,758,231, 1,755,069, 1,744,810, 1,755,063, 1,757,505, 1,757,508, 1,758,193, 1,692,958, and 1,738,179 by free uptake at 5000 nM, 1000 nM, 200 nM, 40 nM, 8 nM, 1.6 nM, 0.32 nM, 0.064 nM, 0.0128 nM, and 0.00256 nM. After a treatment period of approximately 96 hours, total RNA was isolated from cells, and subjected to DGE (Digital Gene Expression) analysis for 3′ end transcriptome profiling using the QuantSeq 3′ mRNA-Seq Library Prep Kit FWD for Illumina on the Illumina sequencing platform. DGE analysis resulted in the generation of 2 million unique, mapped reads, with expression data for 9,496 unigenes. Knockdown was measured as a concentration response experiment and IC₅₀s was determined for concentration responsive genes demonstrating knockdown. Reduction of on-target (APOC3) mRNA was compared to the IC₅₀ of all concentration responsive genes that demonstrated knockdown. The number of differentially expressed off-target genes detected with each oligomeric duplex treatment were identified as critical responders (off-target IC50 within 10× of on-target IC50 and at least 50% reduction) or responders (at least 50% reduction). Treatment with any of compound nos. 1754976, 1758231, 1755069, 1744810, 1755063, 1757505, 1757508, 1758193, 1692958, and 1738179 resulted in no differentially expressed genes with IC50s within 10× of the on-target APOC3 IC50. For compound nos. 1754976, 1758231, 1755069, 1744810, 1755063, 1757505, 1757508, 1758193, and 1738179 there were 1 to 15 differentially expressed genes that demonstrated at least 50% off-target reduction. Treatment with compound 1692958 resulted in 34 differentially expressed genes that demonstrated at least 50% reduction. Treatment with any of compound nos. 1758231, 1755069, 1744810, 1757505, 1757508, or 1758193 did not result in any differentially expressed genes with IC50s within 100× of the on-target APOC3 IC50, whereas compound nos 1754976, 1755063, 1738179 resulted in at least one differentially expressed gene with IC50 within 100× of the on-target APOC3 IC50.

### Example 9: Effect of Oligomeric Duplex Compounds Targeting Human APOC3 in Non-Human Primates

Non-human primates (Macaca fascicularis monkeys from NafoVanny (Tam Phuoc Hamlet, Bien Hoa City, Dong Nai Province, Vietnam)) were treated with oligomeric duplex compound 1758231 or 1738179. Protocols were approved by the Testing Facility's Institutional Animal Care and Use Committee (IACUC). Prior to the study, cynomolgus monkeys 2-4 years old and weighing 2-4 kg were kept in quarantine during which the animals were observed daily for general health. During the study period, monkeys were observed for signs of illness or distress. Any animal showing signs of severe debility or toxicity, particularly if death appears imminent, were euthanized for humane reasons as soon as possible with attending veterinarian consultation.

Two groups of 4 randomly assigned monkeys (2 male and 2 female) were injected subcutaneously with either 1758231 or 1738179. Each monkey was dosed once per 4 weeks on Day 1 and Day 28 with either 4 mg/kg or 50 mg/kg of either 1758231 or 1738179 as indicated. A control group (2 male and 2 female monkeys) was injected with saline in a similar manner. Scheduled euthanasia of animals was conducted on Day 31, approximately 72 hours after the last dose. RNA was extracted from liver for real-time PCR analysis of mRNA expression of APOC3. APOC3 RNA was analyzed using the primer probe set RTS1392, and APOC3 RNA levels normalized to total RNA as measured by RIBOGREEN®. APOC3 RNA was also analyzed using the primer probe set Mf02794312_m1 (Thermo Fisher), which had better match to the monkey sequence, and APOC3 RNA levels normalized to total RNA quantified using RIBOGREEN®. Results are presented in Table 32 as percent APOC3 RNA relative to the amount of APOC3 RNA in control animals, (% control).

TABLE 32
Inhibition of Cynomolgus APOC3 compared to the Saline control

| | | | APOC3 RNA | APOC3 RNA |
|---|---|---|---|---|
| Compound | Subject | Dose | (% Control) | (% Control) |
| No. | Sex | (mg/kg) | RTS1392 | Mf02794312_m1 |
| Saline | Male | — | 103% | 132 |
| | Male | | 97% | 68 |
| | Female | | 84% | 77 |
| | Female | | 116% | 123 |
| 1758231 | Male | 4 | 12% | 18 |
| | Male | | 5% | 5 |
| | Female | | 28% | 28 |
| | Female | | 32% | 31 |
| | Male | 50 | 4% | 4 |
| | Male | | 5% | 8 |
| | Female | | 8% | 12 |
| | Female | | 7% | 14 |
| 1738179 | Male | 4 | 8% | 7 |
| | Male | | 11% | 9 |
| | Female | | 34% | 21 |
| | Female | | 26% | 31 |

1.-95. (canceled)

96. An oligomeric agent comprising an oligomeric compound according to any one of the following chemical notation:
(SEQ ID NO: 11)
VP-TesCfsAyoCyoUyoGfoAyoGyoAyoAyoUyoAyoCyoUfoGyoUfoCyoCyoCyoUyoUysUysUy,
(SEQ ID NO: 12)
VP-TesCfsAyoCyoUyoGdoAyoGyoAyoAyoUyoAyoCyoUfoGyoUfoCyoCyoCyoUyoUysUysUy,
(SEQ ID NO: 13)
VP-TesCfsAyoCyoUyoGdsAyoGyoAyoAyoUyoAyoCyoUfoGyoUfoCyoCyoCyoUyoUysUysUy,
(SEQ ID NO: 14)
VP-TesCfsAyoCyoUyoGdoAyoGyoAyoAyoUyoAyoCyoUdoGyoUdoCyoCyoCyoUyoUysUysUy,
(SEQ ID NO: 15)
VP-TesCfsAyoCyoUyoGdsAyoGyoAyoAyoUyoAyoCyoUdsGyoUdsCyoCyoCyoUyoUysUysUy,
(SEQ ID NO: 16)
VP-TesCfsAyoCyoUyoGyoAyoGyoAyoAyoUyoAyoCyoUfoGyoUyoCyoCyoCyoUyoUysUysUy,
(SEQ ID NO: 17)
VP-TesCfsAyoCyoUyoGfoAyoGyoAeoAyoUyoAyoCyoUfoGyoUfoCyoCyoCyoUyoUysUysUy,
(SEQ ID NO: 18)
VP-TesCfsAyoCyoUyoGfoAyoGyoAeoAeoUyoAyoCyoUfoGyoUfoCyoCyoCyoUyoUysUysUy,
(SEQ ID NO: 19)
mP-TesCfsAyoCyoUyoGfoAyoGyoAyoAyoUyoAyoCyoUfoGyoUfoCyoCyoCyoUyoUysUysUy,
(SEQ ID NO: 20)
VP-TesCfsAyoCyoUyoGdsAyoGyoAyoAyoUyoAyoCyoUfoGyoUfoCyoCyoCyoUyoUysAesAe,
(SEQ ID NO: 21)
VP-TesCfsAyoCyoUyoGdsAyoGyoAyoAyoUyoAyoCyoUdsGyoUdsCyoCyoCyoUyoUysAesAe,
(SEQ ID NO: 22)
VP-TesCfsAyoCyoUyoGdoAyoGyoAyoAyoUyoAyoCyoUfoGyoUfoCyoCyoCyoUyoUysAesAe,
(SEQ ID NO: 23)
VP-TesCfsAyoCyoUyoGdoAyoGyoAyoAyoUyoAyoCyoUdoGyoUdoCyoCyoCyoUyoUysAesAe,
(SEQ ID NO: 24)
VP-TesCfsAyoCyoUyoGdsAyoGyoAeoAyoUyoAyoCyoUfoGyoUfoCyoCyoCyoUyoUysAesAe,
(SEQ ID NO: 25)
VP-TesCfsAyoCyoUyoGdoAyoGyoAeoAyoUyoAyoCyoUdoGyoUdoCyoCyoCyoUyoUysAesAe,
(SEQ ID NO: 26)
VP-TesCfsAyoCyoUyoGdoAyoGyoAcoAeoUyoAyoCyoUdoGyoUdoCyoCyoCyoUyoUysAesAe,
(SEQ ID NO: 27)
VP-TesCfsAyoCyoUyoGdoAyoGyoAeoAyoUyoAyoCyoUfoGyoUfoCyoCyoCyoUyoUysAesAe,
(SEQ ID NO: 28)
VP-TesCfsAyoCyoUyoGdoAyoGyoAeoAyoUyoAyoCyoTdoGyoTdoCyoCyoCyoUyoUysAesAe,
(SEQ ID NO: 29)
VP-TesCfsAyoCyoUyoGdsAyoGyoAeoAyoUyoAyoCyoUdsGyoUdsCyoCyoCyoUyoUysAesAe,
(SEQ ID NO: 30)
VP-TesCfsAyoCyoUyoGdoAyoGyoAeoAeoUyoAyoCyoUfoGyoUfoCyoCyoCyoUyoUysAesAe,
(SEQ ID NO: 31)
VP-TesCfsAyoCyoUyoGdsAyoGyoAeoAyoUyoAyoCyoTdsGyoTdsCyoCyoCyoUyoUysAesAe,
(SEQ ID NO: 32)
VP-TesCfsAyoCyoUyoGfoAyoGyoAyoAyoUyoAyoCyoUfoGyoUfoCyoCyoCysAesAe,
(SEQ ID NO: 33)
VP-TesCfsAyoCyoUyoGdsAyoGyoAyoAyoUyoAyoCyoUfoGyoUfoCyoCyoCysAesAe,
and
(SEQ ID NO: 34)
VP-TesCfsAyoCyoUyoGdsAyoGyoAyoAyoUyoAyoCyoUdsGyoUdsCyoCyoCysAesAe,
(SEQ ID NO: 51)
TesCfsAyoCyoUyoGfoAyoGyoAyoAyoUyoAyoCyoUfoGyoUfoCyoCyoCyoUyoUysUysUy,
(SEQ ID NO: 52)
TesCfsAyoCyoUyoGdoAyoGyoAyoAyoUyoAyoCyoUfoGyoUfoCyoCyoCyoUyoUysUysUy,
(SEQ ID NO: 53)
TesCfsAyoCyoUyoGdsAyoGyoAyoAyoUyoAyoCyoUfoGyoUfoCyoCyoCyoUyoUysUysUy,
(SEQ ID NO: 54)
TesCfsAyoCyoUyoGdoAyoGyoAyoAyoUyoAyoCyoUdoGyoUdoCyoCyoCyoUyoUysUysUy,
(SEQ ID NO: 55)
TesCfsAyoCyoUyoGdsAyoGyoAyoAyoUyoAyoCyoUdsGyoUdsCyoCyoCyoUyoUysUysUy,
(SEQ ID NO: 56)
TesCfsAyoCyoUyoGyoAyoGyoAyoAyoUyoAyoCyoUfoGyoUyoCyoCyoCyoUyoUysUysUy,
(SEQ ID NO: 57)
TesCfsAyoCyoUyoGfoAyoGyoAeoAyoUyoAyoCyoUfoGyoUfoCyoCyoCyoUyoUysUysUy,
(SEQ ID NO: 58)
TesCfsAyoCyoUyoGfoAyoGyoAcoAeoUyoAyoCyoUfoGyoUfoCyoCyoCyoUyoUysUysUy,
(SEQ ID NO: 59)
TesCfsAyoCyoUyoGfoAyoGyoAyoAyoUyoAyoCyoUfoGyoUfoCyoCyoCyoUyoUysUysUy,
(SEQ ID NO: 60)
TesCfsAyoCyoUyoGdsAyoGyoAyoAyoUyoAyoCyoUfoGyoUfoCyoCyoCyoUyoUysAesAe,
(SEQ ID NO: 61)
TesCfsAyoCyoUyoGdsAyoGyoAyoAyoUyoAyoCyoUdsGyoUdsCyoCyoCyoUyoUysAesAe,
(SEQ ID NO: 62)
TesCfsAyoCyoUyoGdoAyoGyoAyoAyoUyoAyoCyoUfoGyoUfoCyoCyoCyoUyoUysAesAe,
(SEQ ID NO: 63)
TesCfsAyoCyoUyoGdoAyoGyoAyoAyoUyoAyoCyoUdoGyoUdoCyoCyoCyoUyoUysAesAe,
(SEQ ID NO: 64)
TesCfsAyoCyoUyoGdsAyoGyoAeoAyoUyoAyoCyoUfoGyoUfoCyoCyoCyoUyoUysAesAe,
(SEQ ID NO: 65)
TesCfsAyoCyoUyoGdoAyoGyoAeoAyoUyoAyoCyoUdoGyoUdoCyoCyoCyoUyoUysAesAe,
(SEQ ID NO: 66)
TesCfsAyoCyoUyoGdoAyoGyoAeoAeoUyoAyoCyoUdoGyoUdoCyoCyoCyoUyoUysAesAe,
(SEQ ID NO: 67)
TesCfsAyoCyoUyoGdoAyoGyoAeoAyoUyoAyoCyoUfoGyoUfoCyoCyoCyoUyoUysAesAe,
(SEQ ID NO: 68)
TesCfsAyoCyoUyoGdoAyoGyoAeoAyoUyoAyoCyoTdoGyoTdoCyoCyoCyoUyoUysAesAe,
(SEQ ID NO: 69)
TesCfsAyoCyoUyoGdsAyoGyoAeoAyoUyoAyoCyoUdsGyoUdsCyoCyoCyoUyoUysAesAe,
(SEQ ID NO: 70)
TesCfsAyoCyoUyoGdoAyoGyoAeoAeoUyoAyoCyoUfoGyoUfoCyoCyoCyoUyoUysAesAe,
(SEQ ID NO: 71)
TesCfsAyoCyoUyoGdsAyoGyoAcoAyoUyoAyoCyoTdsGyoTdsCyoCyoCyoUyoUysAesAe,
(SEQ ID NO: 72)
TesCfsAyoCyoUyoGfoAyoGyoAyoAyoUyoAyoCyoUfoGyoUfoCyoCyoCysAesAe,
(SEQ ID NO: 73)
TesCfsAyoCyoUyoGdsAyoGyoAyoAyoUyoAyoCyoUfoGyoUfoCyoCyoCysAesAe,
and
(SEQ ID NO: 74)
TesCfsAyoCyoUyoGdsAyoGyoAyoAyoUyoAyoCyoUdsGyoUdsCyoCyoCysAesAe;
wherein:
A=an adenine nucleobase,
C=a cytosine nucleobase,
G=a guanine nucleobase,
T=a thymine nucleobase,
U=a uracil nucleobase,
d=a 2′-β-D-deoxyribosyl sugar moiety,
e=a 2′-MOE sugar moiety,
f=a 2′-fluoro sugar moiety,
y=a 2′-OMe sugar moiety,
o=a phosphodiester internucleoside linkage,
s=a phosphorothioate internucleoside linkage,
VP=a 5′ vinyl phosphonate moiety, and
mP=methylene phosphonate.

97. An oligomeric agent comprising an oligomeric compound according to any one of the following chemical notation:
(SEQ ID NO: 35)
THA-GalNAc-AysAysGyoGyoGyoAyoCfoAyoGfoUfoAfoUyoUyoCyoUyoCyoAyoGyoUysGysAy,
(SEQ ID NO: 36)
THA-GalNAc-AysAysGyoGyoGyoAyoCyoAyoGyoUfoAfoUyoUyoCyoUyoCyoAyoGyoUysGysAy,
(SEQ ID NO: 37)
THA-GalNAc-AysAysGyoGyoGyoAyoCyoAfoGyoUfoAyoUfoUyoCyoUyoCyoAyoGyoUysGysAy,
(SEQ ID NO: 38)
THA-GalNAc-AysAysGyoGyoGyoAyoCyoAyoGyoUfsAfoUyoUyoCyoUyoCyoAyoGyoUysGysAy,
(SEQ ID NO: 39)
THA-GalNAc-AesAesGyoGyoGyoAyoCyoAyoGyoUfoAfoUyoUyoCyoUyoCyoAyoGyoUysGesAe,
(SEQ ID NO: 40)
THA-GalNAc-AesAesGyoGyoGyoAyoCyoAyoGyoUfsAfoUyoUyoCyoUyoCyoAyoGyoUysGesAe,
(SEQ ID NO: 41)
THA-GalNAc-AesAesGyoGyoGyoAyoCyoAyoGfsUfoAyoUyoUyoCyoUyoCyoAyoGyoUysGesAe,
(SEQ ID NO: 42)
THA-GalNAc-GysGysGyoAyoCyoAyoGfoUyoAfoUfoUfoCyoUyoCyoAyoGyoUysGysAy,
(SEQ ID NO: 43)
THA-GalNAc-GysGysGyoAyoCyoAyoGfoUyoAfoUyoUyoCyoUyoCyoAyoGyoUysGysAy,
(SEQ ID NO: 44)
THA-GalNAc-GysGysGyoAyoCyoAyoGyoUfoAyoUfoUyoCyoUyoCyoAyoGyoUysGysAy,
(SEQ ID NO: 45)
THA-GalNAc-GysGysGyoAyoCyoAyoGyoUyoAfoUyoUfoCyoUyoCyoAyoGyoUysGysAy,
(SEQ ID NO: 46)
THA-GalNAc-GesGesGyoAyoCyoAyoGyoUyoAyoUfsUfoCyoUyoCyoAyoGyoUysGesAe,
(SEQ ID NO: 47)
THA-GalNAc-GesGesGyoAyoCyoAyoGyoUyoAfsUfoUyoCyoUyoCyoAyoGyoUysGesAe,
(SEQ ID NO: 48)
THA-GalNAc-TdoAysAysGyoGyoGyoAyoCfoAyoGfoUfoAfoUyoUyoCyoUyoCyoAyoGyoUysGysAy,
and
(SEQ ID NO: 49)
AysAysGyoGyoGyoAyoCfoAyoGfoUfoAfoUyoUyoCyoUyoCyoAyoGyoUysGysAy-HPPO-GalNAc;
wherein: 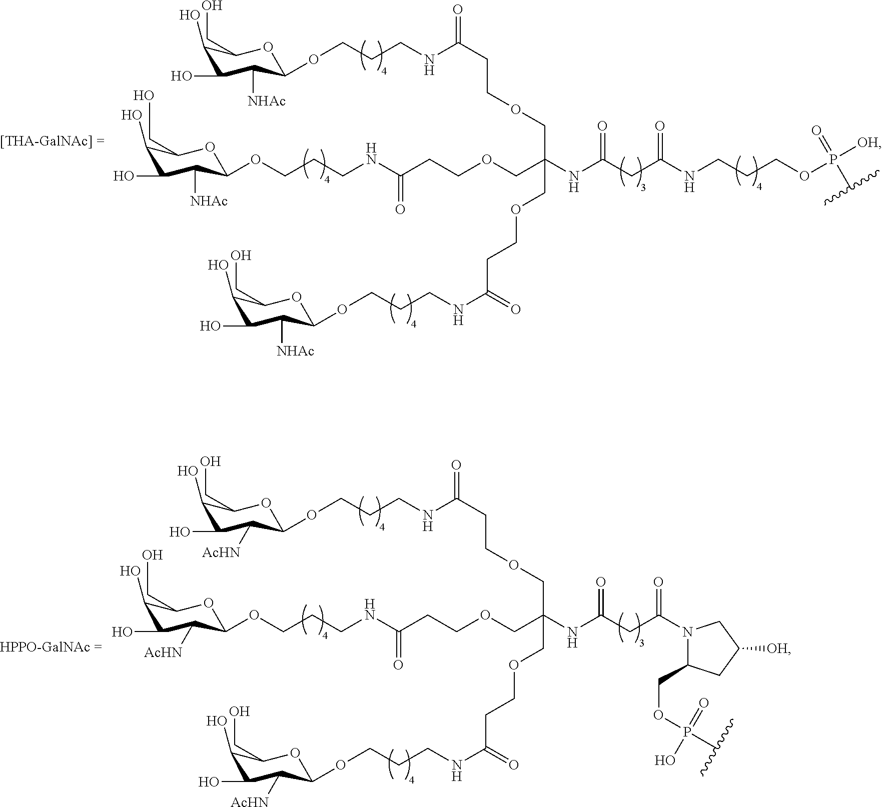
A=an adenine nucleobase,
C=a cytosine nucleobase,
G=a guanine nucleobase,
T=a thymine nucleobase,
U=a uracil nucleobase,
d=a 2′-β-D-deoxyribosyl sugar moiety,
e=a 2′-MOE sugar moiety,
f=a 2′-fluoro sugar moiety,
y=a 2′-OMe sugar moiety,
o=a phosphodiester internucleoside linkage, and
s=a phosphorothioate internucleoside linkage.

98. An oligomeric agent comprising an oligomeric compound according to any one of the following chemical notation:
(SEQ ID NO: 75)
AysAysGyoGyoGyoAyoCfoAyoGfoUfoAfoUyoUyoCyoUyoCyoAyoGyoUysGysAy,
(SEQ ID NO: 76)
AysAysGyoGyoGyoAyoCyoAyoGyoUfoAfoUyoUyoCyoUyoCyoAyoGyoUysGysAy,
(SEQ ID NO: 77)
AysAysGyoGyoGyoAyoCyoAfoGyoUfoAyoUfoUyoCyoUyoCyoAyoGyoUysGysAy,
(SEQ ID NO: 78)
AysAysGyoGyoGyoAyoCyoAyoGyoUfsAfoUyoUyoCyoUyoCyoAyoGyoUysGysAy,
(SEQ ID NO: 79)
AesAesGyoGyoGyoAyoCyoAyoGyoUfoAfoUyoUyoCyoUyoCyoAyoGyoUysGesAe,
(SEQ ID NO: 80)
AesAesGyoGyoGyoAyoCyoAyoGyoUfsAfoUyoUyoCyoUyoCyoAyoGyoUysGesAe,
(SEQ ID NO: 81)
AesAesGyoGyoGyoAyoCyoAyoGfsUfoAyoUyoUyoCyoUyoCyoAyoGyoUysGesAe,
(SEQ ID NO: 82)
GysGysGyoAyoCyoAyoGfoUyoAfoUfoUfoCyoUyoCyoAyoGyoUysGysAy,
(SEQ ID NO: 83)
GysGysGyoAyoCyoAyoGfoUyoAfoUyoUyoCyoUyoCyoAyoGyoUysGysAy,
(SEQ ID NO: 84)
GysGysGyoAyoCyoAyoGyoUfoAyoUfoUyoCyoUyoCyoAyoGyoUysGysAy,
(SEQ ID NO: 85)
GysGysGyoAyoCyoAyoGyoUyoAfoUyoUfoCyoUyoCyoAyoGyoUysGysAy,
(SEQ ID NO: 86)
GesGesGyoAyoCyoAyoGyoUyoAyoUfsUfoCyoUyoCyoAyoGyoUysGesAe,
(SEQ ID NO: 87)
GesGesGyoAyoCyoAyoGyoUyoAfsUfoUyoCyoUyoCyoAyoGyoUysGesAe,
(SEQ ID NO: 88)
TdoAysAysGyoGyoGyoAyoCfoAyoGfoUfoAfoUyoUyoCyoUyoCyoAyoGyoUysGysAy,
and
(SEQ ID NO: 89)
AysAysGyoGyoGyoAyoCfoAyoGfoUfoAfoUyoUyoCyoUyoCyoAyoGyoUysGysAy,
wherein:
A=an adenine nucleobase,
C=a cytosine nucleobase,
G=a guanine nucleobase,
T=a thymine nucleobase,
U=a uracil nucleobase,
d=a 2′-β-D-deoxyribosyl sugar moiety,
e=a 2′-MOE sugar moiety,
f=a 2′-fluoro sugar moiety,
y=a 2′-OMe sugar moiety,
o=a phosphodiester internucleoside linkage, and
s=a phosphorothioate internucleoside linkage.

99. An oligomeric duplex comprising a first oligomeric compound and a second oligomeric compound, wherein the first oligomeric compound comprises VP-TesCfsAyoCyoUyoGdsAyoGyoAeoAyoUyoAyoCyoTdsGyoTdsCyoCyoCyoUyoUysAesAe (SEQ ID NO: 31), wherein:
A=an adenine nucleobase,
C=a cytosine nucleobase,
G=a guanine nucleobase,
T=a thymine nucleobase,
U=a uracil nucleobase,
d=a 2′-β-D-deoxyribosyl sugar moiety,
e=a 2′-MOE sugar moiety,
f=a 2′-fluoro sugar moiety,
y=a 2′-OMe sugar moiety,
o=a phosphodiester internucleoside linkage,
s=a phosphorothioate internucleoside linkage, and
VP=a 5′ vinyl phosphonate moiety, and
wherein the second oligomeric compound comprises THA-GalNAc-AesAesGyoGyoGyoAyoCyoAyoGyoUfsAfoUyoUyoCyoUyoCyoAyoGyoUysGesAe (SEQ ID NO: 40), wherein: 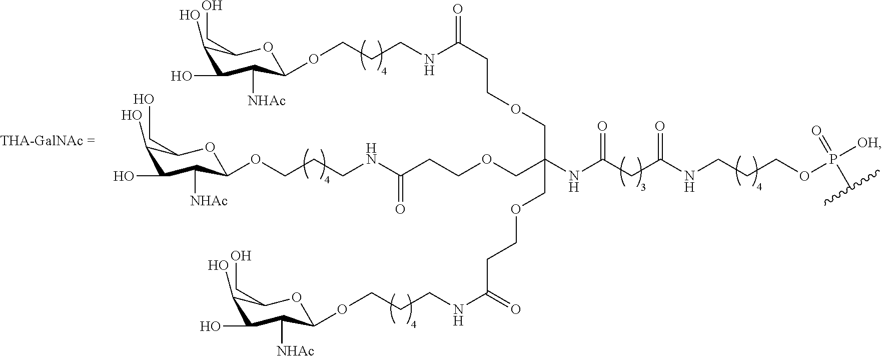
A=an adenine nucleobase,
C=a cytosine nucleobase,
G=a guanine nucleobase,
U=a uracil nucleobase,
d=a 2′-β-D-deoxyribosyl sugar moiety,
e=a 2′-MOE sugar moiety,
f=a 2′-fluoro sugar moiety,
y=a 2′-OMe sugar moiety,
o=a phosphodiester internucleoside linkage, and
s=a phosphorothioate internucleoside linkage.

100. An oligomeric duplex comprising a first oligomeric compound and a second oligomeric compound,
wherein the first oligomeric compound comprises TesCfsAyoCyoUyoGdsAyoGyoAeoAyoUyoAyoCyoTdsGyoTdsCyoCyoCyoUyoUysAesAe (SEQ ID NO: 71), wherein:
A=an adenine nucleobase,
C=a cytosine nucleobase,
G=a guanine nucleobase,
T=a thymine nucleobase,
U=a uracil nucleobase,
d=a 2′-β-D-deoxyribosyl sugar moiety,
e=a 2′-MOE sugar moiety,
f=a 2′-fluoro sugar moiety,
y=a 2′-OMe sugar moiety,
o=a phosphodiester internucleoside linkage, and
s=a phosphorothioate internucleoside linkage, and
wherein the second oligomeric compound comprises AesAesGyoGyoGyoAyoCyoAyoGyoUfsAfoUyoUyoCyoUyoCyoAyoGyoUysGesAe (SEQ ID NO: 80), wherein:
A=an adenine nucleobase,
C=a cytosine nucleobase,
G=a guanine nucleobase,
U=a uracil nucleobase,
e=a 2′-MOE sugar moiety,
f=a 2′-fluoro sugar moiety,
y=a 2′-OMe sugar moiety,
o=a phosphodiester internucleoside linkage, and
s=a phosphorothioate internucleoside linkage.

101. An oligomeric duplex according to the following chemical structure: 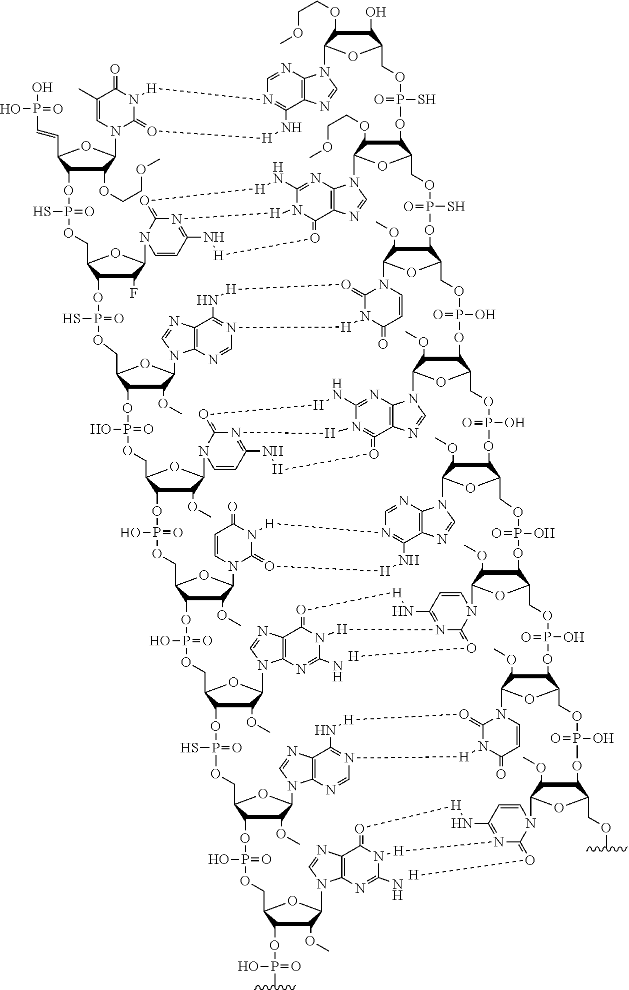 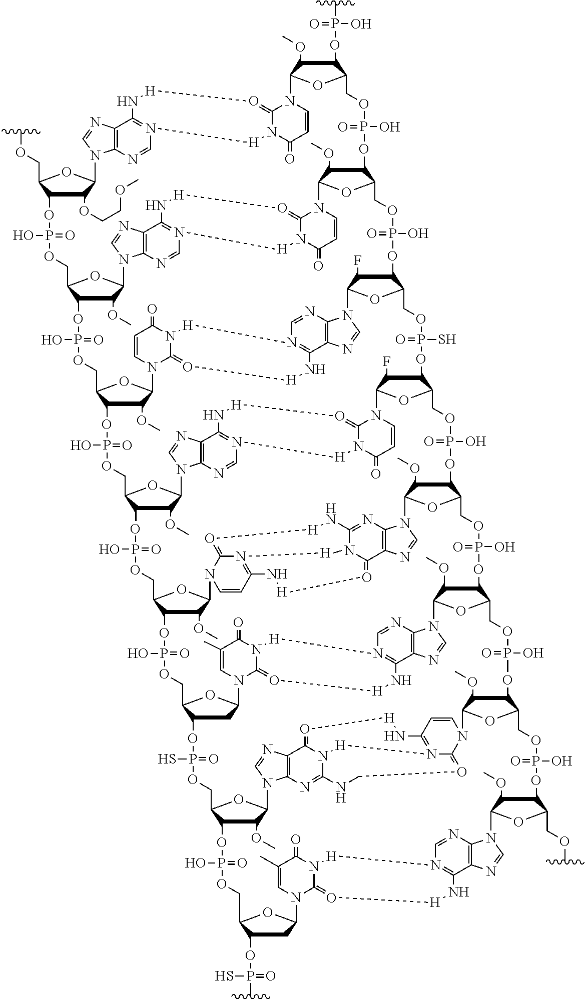 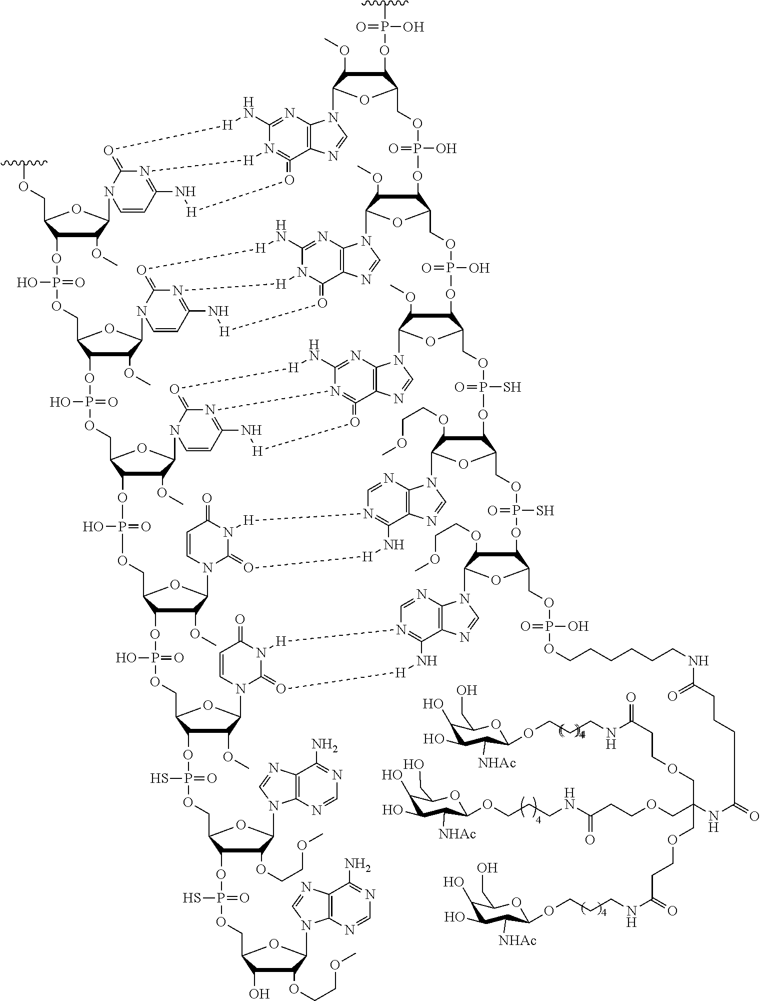
(SEQ ID NO: 31 and SEQ ID NO: 40), or an ion or salt thereof.

102. The oligomeric duplex of claim 101, which is the sodium salt or potassium salt.

103. An oligomeric duplex according to the following chemical structure: 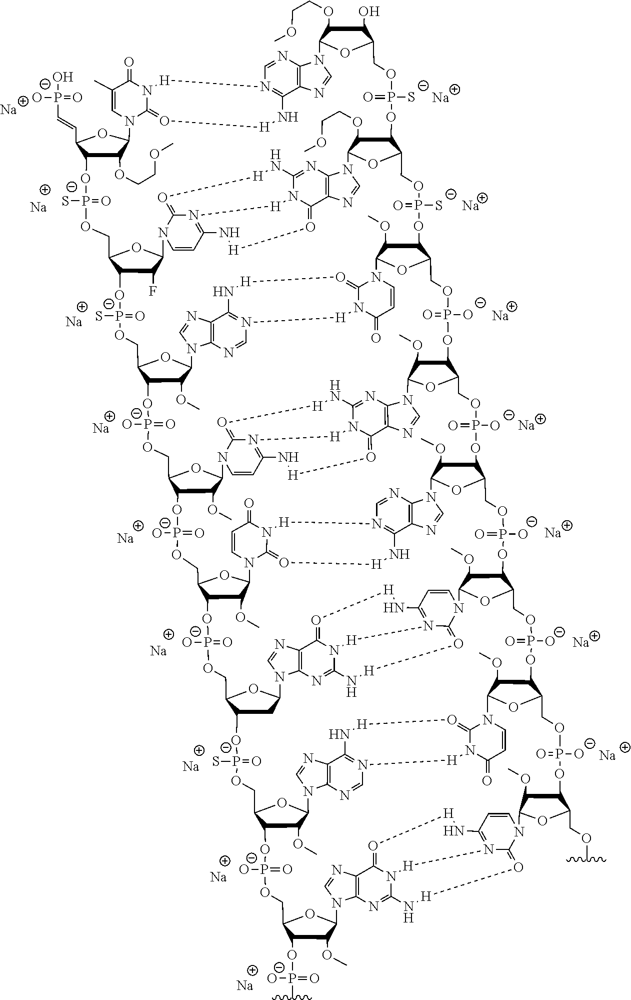 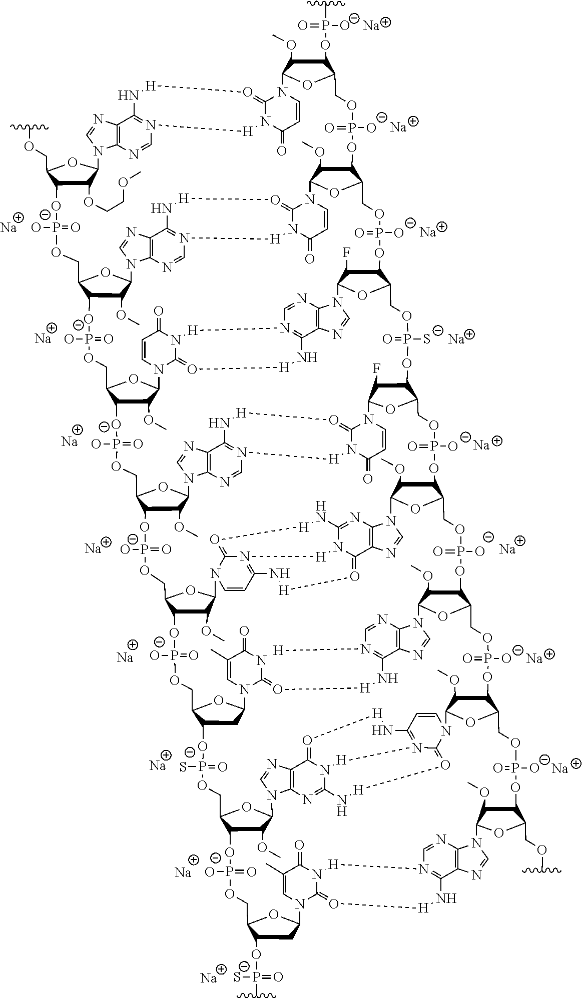 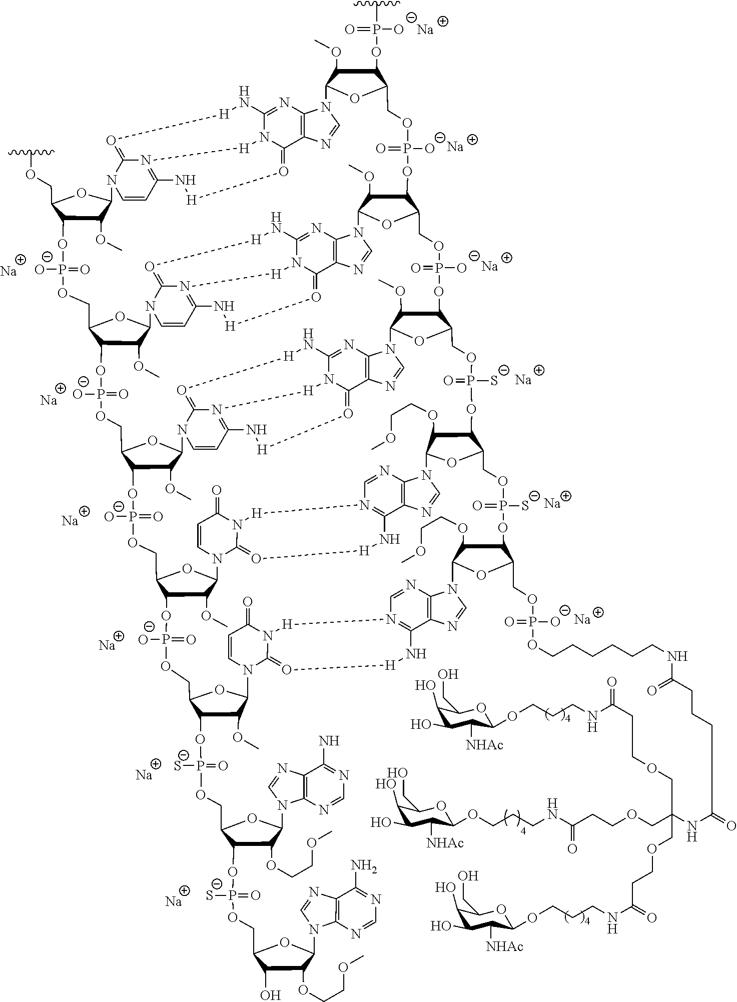
(SEQ ID NO: 31 and SEQ ID NO: 40).

104.-136. (canceled)

137. A method of treating a cardiovascular disease, disorder or condition,
comprising administering to a subject having, or at risk of having, a cardiovascular disease, disorder, or condition, an oligomeric agent of claim 96,
wherein the disease, disorder, or condition is a dyslipidemia, atherosclerotic cardiovascular disease (ASCVD), and/or coronary artery disease (CAD).

138.-166. (canceled)

167. The oligomeric duplex of claim 100, wherein the duplex comprises a conjugate group.

168. The oligomeric duplex of claim 167, wherein the conjugate group comprises a cell-targeting moiety.

169. The oligomeric duplex of claim 168, wherein the conjugate group comprises a liver cell targeting moiety.

170. The oligomeric duplex of claim 169, wherein the conjugate moiety is a GalNAc moiety.

171. The oligomeric duplex of claim 167, wherein the second oligomeric compound comprises the conjugate group conjugated directly to the second modified oligonucleotide.

172. The oligomeric duplex of claim 171, wherein the conjugate group is conjugated to the 5′ end or 3′ end of the second modified oligonucleotide.

173. The oligomeric duplex of claim 172, wherein the conjugate group is attached to the 5′-terminal nucleoside of the second modified oligonucleotide.

174. The oligomeric duplex of claim 172, wherein the conjugate group is attached to the 3′-terminal nucleoside of the second modified oligonucleotide.

175. A pharmaceutical composition comprising the oligomeric duplex of claim 101 and a pharmaceutically acceptable diluent or carrier.

176. The pharmaceutical composition of claim 175, wherein the pharmaceutically acceptable diluent is water or phosphate-buffered saline.

177. The pharmaceutical composition of claim 175, wherein the pharmaceutical composition consists essentially of the oligomeric duplex, and water or phosphate-buffered saline.

178. A pharmaceutical composition comprising the oligomeric duplex of claim 103, and a pharmaceutically acceptable diluent or carrier.

179. The pharmaceutical composition of claim 178, wherein the pharmaceutically acceptable diluent is water or phosphate-buffered saline.

180. The pharmaceutical composition of claim 178, wherein the pharmaceutical composition consists essentially of the oligomeric duplex, and water or phosphate-buffered saline.

181. The oligomeric duplex of claim 100, wherein the first oligomeric compound comprises a stabilized phosphate group attached to the 5′-terminal nucleoside, and wherein the stabilized phosphate group comprises a methylene phosphonate, cyclopropyl phosphonate or a vinyl phosphonate.
